(19) United States
(12) Patent Application Publication
(10) Pub. No.: US 2008/0108101 A1
(43) Pub. Date: May 8, 2008

(54) Novel Classification Method of Blood Cells and Tailor-Made Therapy and Prevention Based Thereupon

(75) Inventor: Toshiyasu Miyazaki, Kawasaki-shi (JP)

Correspondence Address: SEED INTELLECTUAL PROPERTY LAW GROUP PLLC, 701 FIFTH AVE, SUITE 5400, SEATTLE, WA, 98104, US

(73) Assignee: H.J.L., Inc., Kawasaki-shi (JP)

(21) Appl. No.: 11/661,668

(22) PCT Filed: Aug. 31, 2005
(86) PCT No.: PCT/IB05/52856

§ 371 Date: Dec. 19, 2007

(30) Foreign Application Priority Data
Sep. 1, 2004 (JP) 2004-254890

(51) Int. Cl. C12Q 1/02

(52) U.S. Cl. 435/29

(57) ABSTRACT

A diagnostic technology to provide more efficient treatment for diagnosis of diseases such as leukemia, is provided according to the present invention. The present invention provides a method for identifying a stage of differentiation-maturation of a cell comprising the steps of: A) measuring an expression level of at least one cellular marker; and 10 B) determining the stage of differentiation-maturation of the cell based on the expression level. The present invention further provides a method for treating a subject based on a differentiation-maturation stage of a cell, comprising the steps of: A) measuring an expression level of at least one cellular marker; B) determining the differentiation-maturation stage of the cell based on the expression level; and C) providing to the subject an appropriate treatment based on the determined differentiation-maturation stage.

### TECHNICAL FIELD

The present invention relates to a method for classification of a cell. More specifically, the present invention is related to a method for classifying a cell in detail according to the level of differentiation thereof, and related technology thereof.

### BACKGROUND ART

Flow cytometry technology has recently been used to analyze a variety of cells. In particular, flow cytometry technology has allowed determination of blood cell differentiation. Such determination of differentiation has been applied to the analysis of leukemia cells, and has also been applied to diagnosis.

Advantages of cytometry include, for example, ease of calculating the ratio of blast cells, high specificity and sensitivity, high reproducibility, capability to analyze a number of cells, the short time required for analysis, and the like. However, on the other hand, there is no antigen specific to leukemia or leukemia cells, and there are very few antigens specific to a certain lineage. Further, the pattern or density of expression of antigens varies depending on the leukemia cell, making it difficult to analyze data to determine different types of leukemia.

In conventional leukemia cell analysis, panels have been produced based on the premise of analyzing lineages of leukemia cells. A variety of CD antigens are combined for use in the production of such panels. The combination of these CD antigens has allowed classification of pre-B-ALL, pre-A-ALL, B-CLL, AML, mixed lineage leukemia, and non-Hodgkin lymphoma. For example, combinations of B lymphocyte antigen and T lymphocyte antigen, and lymphoid antigen and bone-marrow antigen, and the like, have been proposed.

However, these CD antigens are also expressed in a normal cell. Pretreatment samples, in which most of the cells are leukemia cells, may be diagnosed with this method. However, in a post treatment sample, in which leukemia cells are decreased, it is difficult to distinguish between normal cells and leukemia cells, and indeed, it is thus essentially impossible to use prior art cytometric methods to analyze post treatment samples.

In a normal blood sample, there are a variety of cells, depending on their respective stage of differentiation or maturation. However, there have not previously been technologies which can accurately determine such differentiation or maturation.

Conventionally, hematopoietic stem cell transplantation therapy has been carried out. However, as concentrates of naturally occurring cells were used, a number of adverse effects have been reported. For example, there have been side effects arising from transplantation pretreatment using a large amount of anticancer agents or radiation (PRT). Moreover, there have been a variety of side effects or adverse effects such as bacterial or fungal infectious diseases, arising from bone marrow suppression during pre-treatment; graft-versus-host disease (GVHD), which occurs when transplantation is derived from others, as the host will regard the patient's organ as a “foreign” (or heterologous) challenge the host, when the leukocytes of the donor have grown and increased; a variety of lung complication syndromes such as cytomegalovirus (CMV) pneumonia; a variety of visceral disorders arising from the disorder of vascular endothelial cell (cells covering inside of blood vessels); a variety of infectious diseases occurring during the immunosuppression stage (for about one to two years) prolonged after incorporation into the body; chronic GVHD exhibiting a variety of syndromes as a consequence of prolongation; secondary cancer; hypogonadism; late or delayed disorders such as infertility or the like.

Contrary to it's original purpose, transplantation often causes temporal deterioration of general condition of the body due to complications. Further, some patients who will die of such complications, are counted for about 10-20% for autologous transplantation, and about 20-40% for heterologous transplantation. Further, even if such complications are overcome, there is possibility that the original disease will recur or relapse. As such, there are limitations to present transplantation therapies.

A method of preventing early death from complex diseases after bone marrow transplantation, comprising separating and isolating stem cells, a method of producing a large amount of precursor cells from the stem cells ex vivo, and a method of treating a disease by transplanting the precursor cells together with the stem cells, have been attempted and clinical trials using the same have been conducted.

FACS (fluorescence activated cell sorter) has been available since the 1980's, and has been utilized for a method of concentration or purification of hematopoietic stem cells. It has been clarified that highly purified hematopoietic stem cells can be obtained by purifying CD34⁻KSL cells from bone marrow cells by multi-color staining. For such differentiated cells, a variety of markers have been used. However, selection by such markers does not correlate 100% to transplantation compatibility or isolation of undifferentiated stem cells. Therefore, it is suggested that such selection is not always sufficient for therapeutic effects.

A variety of proteins play important roles in the machinery regulating the differentiation of a stem cell. For example, stem cell factor (or steel factor; SCF) is a notable factor for hematopoietic stem cells.

SCF is produced by bone marrow stromal cells and acts on pluripotent stem cells, bone marrow cells (e.g., CFU-M, CFU-GM, CFU-Meg, etc.), and lymphocyte precursor cells to support their expansion and differentiation.

That is, it is believed that SCF acts on cells from hematopoietic stem cells to precursor cells so as to aid other cytokines which induce differentiation toward the final stage (S. Kitamura, Saitokain-no-Saizensen [Frontline of Cytokine], Yodo-sha, edited by T. Hirano, pp. 174-187, 2000). However, such a factor cannot be a marker.

Cell surface markers such as CD34, Lin (lineage marker), c-kit and Sca-1 and the like have been generally used to identify a stem cells. However, there are a number of drawback as these markers are not particularly specific, and are not sufficient for determining transplantation compatibility, undifferentiated state of the stem cells, and the like. Even if these markers are combined, it is questionable whether stem cells can be separated to nearly 100% purity. In particular, when considering transplantation compatibility, even if all the markers are used, only a mixture of cells comprising cells having transplantation compatibility and those having no such compatibility is obtained. This is a reason why there are some patients who have successfully undergone bone marrow transplantation and those who have not successfully undergone the same. In particular, with respect to stem cell characteristics and transplantation compatibility of hematopoietic stem cells, it can be said that there is currently no appropriate marker.

Multi dimensional flow cytometry (MDF), a bone marrow monitoring test, which is a new useful detection method for hematopoietic tumors (leukemia, lymphoma, and a variety of other blood diseases), has resulted, in most cases, in the palliation of the above disorders due to recent advances in the filed of hematopoietic tumor therapy.

However, morbidity due to recurrence is still high, and thus the overall cure rate still remains low. If monitoring could be conducted in detail for the blood cells of a patient during each course of therapy, it would be extremely useful in the clinical setting. At present, analyses of hematopoietic tumor cells using FCM being conducted in Japan include: 1) case classification before treatment; 2) stem cell quantification necessary for stem cell transplantation, and the like. However, 3) assessment of therapeutic effects by monitoring tumor cells in the patient, or 4) detection or medical check for prognosis or prevention of recurrence, have not yet been established. The use of MDF allows analyses focusing on whether or not 1) transplanted hematopoietic stem cells have been taken or incorporated in the body; 2) bone marrow has been properly reconstructed; 3) detecting whether abnormal progress of bone marrow cells has occurred, which is indicative of recurrence. Hematopoietic tumor analyses using FCM have been conducted inside Japan. However, these are only focusing on case classification before treatment, and cannot be used for to monitor the effects of treatment. Further, most of these specimens provide data that is useless for clinical purposes by detection/medical check center run by major incorporations only chasing efficiency and automation. IT is of great concern that a number of hematopoietic tumor patients have been dying as a direct or indirect result of such useless data.

At present, it is said that, in Japan, there are some 30,000 leukemia patients, and 50,000 lymphoma patients. There are some cases therein for which case classification before treatment is conducted using FCM. However, there are no cases for which post-treatment monitoring has been conducted using FCM. Further, for myelo dysplasia syndrome patients, of which there are said to be of 50,000, cases are never classified using FCM prior to treatment, because there is general recognition that conventional FCM detection is useless in such cases.

### SUMMARY OF INVENTION

### Problem to be Solved

In view of the above problems, it is an object of the present invention to provide diagnostic technology to provide more effective therapy against diseases such as leukemia.

### Means for Solving the Problem

The present invention provides, as a consequence of significant efforts, a solution to solve the above-mentioned problems, by the finding that analysis of the expression level of cellular markers allows more detailed identification of the differentiation/maturation of stem cells or the like, and that there are a number of maturation stages unknown prior to the present invention, and that it is possible to find an appropriate treatment according to such maturation stages.

Thus, the present invention provides the following:

1. A method for identifying a stage of differentiation-maturation of a cell comprising the steps of:

### A) measuring an expression level of at least one cellular marker; and

### B) determining the stage of differentiation-maturation of the cell based on the expression level.

### 2. The method according to Item 1, wherein the expression level of the cellular marker is relative.

### 3. The method according to Item 1, wherein the expression level of the cellular marker is absolute.

### 4. The method according to Item 1, wherein at least two cellular markers are used as the cellular marker.

### 5. The method according to Item 1, wherein at least three cellular markers are used as the cellular marker.

### 6. The method according to Item 1, wherein at least four cellular markers are used as the cellular marker.

7. The method according to Item 1, wherein the cellular markers are selected from the group consisting of: CD1a, CD2, CD3, CD4, CD5, CD7, CD8, CD10, CD11b, CD13, CD14, CD15, CD16, CD19, CD20, CD22, CD23, CD33, CD34, CD45, TdT, FMC7 and HLA-DR.

### 8. The method according to Item 1, wherein the cell comprises a hematopoietic cell.

### 9. The method according to Item 1, wherein the cell comprises a cell selected from the group consisting of monocytic lineage cells, myeloid lineage cells, B lymphocyte-like cells, and T lymphocytic lineage cells.

### 10. The method according to Item 1, wherein the differentiation-maturation stage comprises Stage 1, Stage 2 and Stage 3 for monocytic lineage cells.

### 11. The method according to Item 1, wherein the differentiation-maturation stage comprises Stage 1, Stage 2, Stage 3, Stage 4 and Stage 5 for myeloid lineage cells.

### 12. The method according to Item 1, wherein the differentiation-maturation stage comprises Stage 1, Stage 2, Stage 3 and Stage 4 for B lymphocytic lineage cells.

### 13. The method according to Item 1, wherein the differentiation-maturation stage comprises Stage 1, Stage 2, Stage 3 and Stage 4 for T lymphocytic lineage cells.

### 14. The method according to Item 1, wherein the cellular marker comprises a combination of lineage specific CD antigens.

### 15. The method according to Item 1, wherein the cellular marker comprises a combination of antigens which are not lineage specific CD antigens.

16. The method according to Item 1, wherein the cellular marker is, with respect to B lymphocytic lineage, selected from the group consisting of the combination of CD10, CD19, CD34 and CD45; the combination of CD10, CD20, CD5 and CD45; the combination of CD19, CD23, CD5 and CD45; the combination of CD10, CD20, CD34 and CD45; and the combination of CD5, CD19, CD34 and CD45.
17. The method according to Item 1 wherein the cellular marker is, with respect to T lymphocytic lineage, selected from the group consisting of the combination of CD1a, CD3, CD10 and CD45; the combination of CD4, CD8, CD7 and CD45; the combination of CD1a, CD3, CD34 and CD45; and the combination of CD4, CD8, CD10 and CD45.
18. The method according to Item 1, wherein the cellular marker is, with respect to myeloid lineage or monocytic lineage, selected from the group consisting of the combination of HLA-DR, CD11b, CD10 and CD45; the combination of CD16, CD13, CD33 and CD45; the combination of CD4, CD10, CD34 and CD45; the combination of CD11b, CD13, CD16 and CD45; and the combination of CD10, CD4, CD14 and CD45.
19. A system for identifying a differentiation/maturation stage of a cell, comprising:

### A) means for measuring an expression level of at least one cellular marker; and

### B) means for determining the differentiation/maturation stage of the cell based on the expression level.

### 20. The system according to Item 19, wherein the means for measuring measures a relative level of an expression of a cellular marker.

### 21. The system according to Item 19, wherein the means for measuring measures an absolute level of an expression of a cellular marker.

### 22. The system according to Item 19, wherein the means for measuring comprises an agent specific to a cellular marker.

### 23. The system according to Item 22, wherein the cellular marker comprises at least two cellular markers.

### 24. The system according to Item 22, wherein the cellular marker comprises at least three cellular markers.

### 25. The system according to Item 22, wherein the cellular marker comprises at least four cellular markers.

### 26. The system according to Item 22, wherein the cellular marker is selected from the group consisting of CD1a, CD2, CD3, CD4, CD5, CD7, CD8, CD10, CD11b, CD13, CD14, CD15, CD16, CD19, CD20, CD22, CD23, CD33, CD34, CD45, TdT, FMC7 and HLA-DR.

### 27. The system according to Item 19, wherein the cell comprises a hematopoietic stem cell.

### 28. The system according to Item 19, wherein the cell comprises a cell selected from the group consisting of a monocytic lineage cell, a B lymphocytic lineage cell, a T lymphocytic lineage cell lineage and a myeloid lineage cell.

### 29. The system according to Item 19, wherein the differentiation/maturation stage comprises Stage 1, Stage 2 and Stage 3 for monocytic lineage.

### 30. The system according to Item 19, wherein the differentiation/maturation stage comprises Stage 1, Stage 2, Stage 3, Stage 4 and Stage 5 for myeloid lineage.

### 31. The system according to Item 19, wherein the differentiation/maturation stage comprises Stage 1, Stage 2, Stage 3 and Stage 4 for B lymphocytic lineage lineage.

### 32. The system according to Item 19, wherein the differentiation/maturation stage comprises Stage 1, Stage 2, Stage 3 and Stage 4 for T lymphocytic lineage lineage.

### 33. The system according to Item 22, wherein the cellular marker comprises a combination of antigens which are lineage specific CD antigens.

### 34. The system according to Item 22, wherein the cellular marker comprises a combination of antigens which are not lineage specific CD antigens.

35. The system according to Item 22, wherein the cellular marker is, with respect to B lymphocytic lineage, selected from the group consisting of the combination of CD10, CD19, CD34 and CD45; the combination of CD10, CD20, CD5 and CD45; the combination of CD19, CD23, CD5 and CD45; the combination of CD10, CD20, CD34 and CD45; and the combination of CD5, CD19, CD34 and CD45.
36. The system according to Item 22, wherein the cellular marker is, with respect to T lymphocytic lineage, selected from the group consisting of the combination of CD1a, CD3, CD10 and CD45; the combination of CD4, CD8, CD7 and CD45; the combination of CD1a, CD3, CD34 and CD45; and the combination of CD4, CD8, CD10 and CD45.
37. The system according to Item 22, wherein the cellular marker is, with respect to myeloid lineage or monocytic lineage, selected from the group consisting of the combination of HLA-DR, CD11b, CD10 and CD45; the combination of CD16, CD13, CD33 and CD45; the combination of CD4, CD10, CD34 and CD45; the combination of CD11b, CD13, CD16 and CD45; and the combination of CD10, CD4, CD14 and CD45.

### 38. The system according to Item 19, wherein the means for measuring comprises a flow cytometer.

39. A method for determining a differentiation/maturation stage of a cell, comprising the steps of:

### A) providing a measurement pattern (“normal pattern”) with respect to a normal cell having a normal differentiation/measurement stage of the cell, in a cytometry of an expression level of at least one cellular marker; and

### B) determining the differentiation/maturation stage of the cell by comparing the pattern during flow cytometry of an expression level of the cellular marker compared with the normal pattern.

40. A system for determining a differentiation/maturation stage of a cell comprising the steps of:

### A) means for providing measurement pattern (“normal pattern”) with respect to a normal cell having a normal differentiation/measurement stage of the cell, in a cytometry of an expression level of at least one cellular marker; and

### B) means for determining the differentiation/maturation stage of the cell by comparing the pattern during flow cytometry of an expression level of the cellular marker compared with the normal pattern.

41. A method for determining whether a differentiation/maturation stage of a cell is normal or not, comprising the steps of:

### A) measuring an expression level of at least one cellular marker in a flow cytometry; and

B) comparing a pattern of the expression level during flow cytometry, with an expression level of the cellular marker presented by a cell in a normal differentiation/maturation stage in a flow cytometry; the fact that there is difference therebetween is indicative of an abnormal cell.
42. A system for determining whether a differentiation/maturation stage of a cell is normal or not, comprising the steps of:

### A) means for measuring an expression level of at least one cellular marker in a flow cytometry; and

B) means for comparing a pattern of the expression level in a flow cytometry, with an expression level of the cellular marker presented by a cell in a normal differentiation/maturation stage in a flow cytometry; the fact that there is difference therebetween is indicative of an abnormal cell.
43. A method for treating a subject based on differentiation/maturation stage of a cell, comprising the steps of:

### A) measuring an expression level of at least one cellular marker;

### B) determining the differentiation/maturation stage of the cell based on the expression level; and

### C) subjecting to the subject an appropriate treatment for the determined differentiation/maturation stage.

44. A method for treating a subject based on a differentiation/maturation stage of a cell, comprising the steps of:

### A) measuring an expression level of at least one cellular marker;

### B) determining the differentiation/maturation stage of the cell based on the expression level; and

### C) subjecting to the subject an appropriate treatment based on the results comparing the determined differentiation/maturation stage, and a stage at which the cell takes in its normal stage.

45. The method according to Item 44, wherein said appropriate treatment comprises:
i) a treatment of continuing the present therapy or termination of the present therapy and to conduct follow-up only, when a normal cell exists or a cell present is determined to be a normal immature cell with respect to the determined differentiation/maturation stage of the cell;
ii) a treatment in which the enhancement or alteration of the present therapy when the present cell is an abnormal cell with respect to the determined differentiation/maturation stage of the cell.
46. The method according to Item 44, wherein the treatment comprises a treatment in which an additional stem cell transplantation is conducted when depletion of the original stem cell or an abnormality of differentiation/maturation progress is recognized, even if the existing cell is a normal immature cell with respect to the determined differentiation/maturation stage of the cell.
47. A system for treating a subject based on differentiation/maturation stage of a cell, comprising the steps of:

### A) means for measuring an expression level of at least one cellular marker;

### B) means for determining the differentiation/maturation stage of the cell based on the expression level; and

### C) means for subjecting to the subject an appropriate treatment for the determined differentiation/maturation stage.

48. A system for providing a subject with an appropriate treatment based on a differentiation/maturation stage of a cell, comprising:

### A) means for measuring an expression level of at least one cellular marker;

### B) means for determining the differentiation/maturation stage of the cell based on the expression level; and

### C) means for providing the subject with an appropriate treatment based on the determined differentiation/maturation stage.

49. A system for treating a subject based on a differentiation/maturation stage of a cell, comprising the steps of:

### A) means for measuring an expression level of at least one cellular marker;

### B) means for determining the differentiation/maturation stage of the cell based on the expression level; and

### C) means for subjecting to the subject an appropriate treatment based on the results comparing the determined differentiation/maturation stage, and a stage at which the cell takes in its normal stage.

50. A system for providing a subject with an appropriate treatment based on a differentiation/maturation stage of a cell, comprising:

### A) means for measuring an expression level of at least one cellular marker;

### B) means for determining the differentiation/maturation stage of the cell based on the expression level; and

### C) means for providing the subject with an appropriate treatment based on the results of comparing the determined differentiation/maturation stage, and a stage at which the cell takes in its normal stage.

### 51. A pattern of differentiation/maturation of a cell produced by a method according to Item 1.

The MDF provided by the present inventors allows prediction of recurrence of leukemia. The present invention also allows accurate assessment of a disease such as leukemia using FCM analysis, which was conventionally believed to be useless. Accordingly, the present invention allows proper and accurate case classification before treatment, using FCM, for leukemia and lymphoma for which there are annually some 30,000 and 50,000 patients, respectively. Further, the present invention allows the provision of sufficient diagnosis results to provide therapeutic guidance for myelo dysplasia syndrome, for which there are some 50,000 patients annually.

Blood cells in bone marrow blood having a normal analysis panel based on the differentiation/maturation pattern of the normal blood cells, have been matured via a normally regulated differentiation process. Particular CD antigens are combined to analyze the quantity and distribution of a variety of antigen contents of normal cells present in bone marrow blood, peripheral blood and lymph node by FCM, allowing presentation of expression and disappearance of each cellular surface antigen as a single lineage. It was revealed by the present invention that this normal pathway is recognized in all blood cells, and regardless of age or therapy subjected to, the same pattern is observed in all humans if the humans have the normal cells.

Analyses of a variety of leukemia cells, based on the data as analyzed in the present invention, showed that leukemia cells never exhibit the same antigen distribution as normal cells. That is, when the expression level of each antigen is measured and plotted on multi-dimensional space, plots of leukemia cells are always located in different spaces to normal cells. This originates from the fact that leukemia cells have antigen expression abnormality (lineage infidelity; antigen expression abnormality, Maturational Asynchrony; Antigenic Absence, and antigen quantitation abnormalities) which the normal bone marrow blood or normal peripheral blood cells do not have. It has been extremely difficult to analyze and determine whether normal immature cells in a stage of differentiation/maturation and leukemia cells are CD antigen positive or negative. However, the present invention has found that an analysis based on the normal pathway allows the detection of a variety of cells.

The Normal Pathway differs depending on the lineage of the cells. B lymphocytes, T lymphocytes, bone marrow cells, monocytes and erythrocytes, and the like, have their own normal pathways and other (abnormal) pathways. These pathways are not affected with individual preference or difference, and thus is stable regardless of such individual preference or difference.

Conventional methods have attempted to define the normal pathways. However, there are detailed pathways which cannot be analyzed and clarified by such methods. The method of the present invention allows the clarification of such detailed pathways.

In conventional methods, the cellular data obtained by flow cytometry (FCM) have cell size (forward scatter; FSC), intracellular structure (side scatter; SSC), antigen fluorescence (fluorescence; FL-1, FL-2, FL-3 and FL-4). In the conventional methods, parameters of FSC×SSC have been analyzed to search for the cells of interest, and observed whether or not each FL of the cells are positive or negative therefor. Monoclonal antibodies (CD) used in the FL, were a combination to classify “T lymphocytic lineage or B lymphocytic lineages” “lymphocytes or bone marrow cells lineages” and the like. These CD have no systematic correlations, and thus meaningful parameters by developing data, may be found in FSC×SSC or FL×FL or the like. Naturally, as tumor cells are not simple, such a classification has never been useful to date. This is also indicated by morphological tests.

In the MDF of the present invention, CD antibodies are related to each cell in a lineage-dependent manner. For example, those which differ in the expression thereof during the differentiation/maturation process of a myeloid lineage cells are CD11b, CD13, CD14, CD15, CD16, CD33, HLA-DR and the like. As all CD's have been expressed in the same lineage cells, all data correlate to one another. Accordingly, these exemplary examples have five data x four data x the number of measured CD, as parameters to be developed. These parameters allow one to analyze the normal pathway in detail.

In an embodiment of the present invention, CDs may be selected with respect to the relationship to the cellular lineage.

Further, it is also possible to analyze the differentiation stages of such lineages in more detail, by analyzing with increased parameters.

(Technical Description of MDF)

Blood cells differentiate and mature from stem cells to a variety of cells. When the process of differentiation and maturation process are analyzed in detail, one can always find one differentiation/maturation lineage (normal pathway). In the present invention, it was revealed that the are no significant differences not only amongst individuals with respect to factors such as age and gender, but also amongst the effects of therapy in this normal pathway. That is, either stem cells which have existed in the body or those transplanted from outside will differentiate along precisely the same lineage. A method for finding the normal pathway is an embodiment of the MDF of the present invention. An example is shown in FIG. 1A.

The definition of a normal Pathway was proposed by Loken. They have analyzed the expression of each of the CD markers during differentiation/maturation from the immature stage to the mature stage of blood cells, and demonstrated that the CD markers regularly and accurately increase or decrease. They have analyzed a variety of cells by three color CD markers for multi-dimensional analyses, and found out the normal pathway. They also found that abnormal cells (tumor cells or the like) appear in a different space from that of cells of the normal pathway, and particularly applied the above method to the detection of minimal residual disease after therapy.

However, the normal pathway which they have found has relied on mostly presumptions, and differentiation/maturation stages that have been identified are inaccurate. This is because of technical problems such as limitations to three-color staining, and the lack of an appropriate combination of CD markers for identifying individual stages in detail. In particular, Loken and others have attempted to detect minimal residual disease, in other words, the same classification or detection of tumor cells which has been conventionally conducted. Therefore, the CD markers specifically selected in their methods were suitable for detecting tumors, but did not allow detailed analysis of pathways.

In the present invention, in order to solve the technical problems described above, multi-dimensional analysis using four types of CD markers has been conducted. This allowed a great increased number of parameters to be analyzed, and greater number of multi-dimensional analyses. CD markers used have been selected to detect a detailed normal pathway, and an appropriate combination has been produced. This allows the classification of the stages of cell populations in detail, which have not been classified with respect to stages by conventional pathway analyses, including, for example, differentiation between stages 2 and 3 in monocytic lineage cells, amongst stages 3, 4 and 5 in myeloid lineage cells, and the like.

Specific measures of MDF are as follows:

1) Transplanted stem cells are always expressed in the position of stem cells. If there is no appearance thereof, there is the possibility that the transplant may fail per se.

2) The appearance of cells differentiating and maturing on the normal pathway and stem cells appearing on the sites of normal stem cells, indicates that bone marrow reconstruction has started.

3) When normal bone marrow reconstruction has advanced, all the variable cells on the differentiation/maturation process from the stem cell, and a mature cell.

4) If all the cells have been matured to differentiated and mature cells, and no stem cells or cells in the process of differentiating/maturing have been recognized, there is the possibility that the transplanted stem cells have not taken and have simply differentiated and matured. As such, stem cells will be depleted, and bone marrow reconstruction will be distinct.

5) If cells in the process of differentiating/maturing have not progressed to advanced maturation, it is believed that bone marrow reconstruction abnormality has occurred at that point in time.

6) Remaining tumor cells will appear at sites completely different from that of normal stem cells. It has been extremely difficult to date to differentiate between tumor cells, immature cells after treatment and normal stem cells. The MDF of the present invention can differentiate and determine the difference between cells by using the difference between these sites.

7) Even if a minor amount of tumor cells are present, if normal bone marrow reconstruction has occurred, no additional potent treatments are necessary as such tumor cells are likely to be cleared by the immune system. However, if a minor amount of tumor cells are present and normal bone marrow reconstruction has not been occurred, there is nothing to prevent the proliferation of tumor cells, and there is thus a risk of recurrence.

The conventional FCM has investigated surface markers of tumor cells to date. However, markers specific to tumor cells have only been recognized in a portion of cases. FCM markers recognized in tumor cells, are also recognized in normal cells in a similar manner. Conventional methods to differentiate between positive or negative, will lead to an identical conclusion.

For example, CD13 is often recognized in myelodysplastic syndrome, whereas CD13 is also recognized in normal stem cells or normal immature bone marrow cells. Conventional methods have reported that “the subject cells are positive for CD13” at this stage. Medical practitioners cannot determine whether this is abnormal or normal.

When using the MDF of the present invention, even the same CD13 positive cell can be distinguished by stating that it is positive on the normal pathway or off the normal pathway. In conclusion, the present invention allows reporting of such a cell as “it is normal” or “it is abnormal”.

The reasons why conventional methods have been as such, is related to the history of FCM. Originally, FCM has been developed as a device for basic research. However, prevalence of AIDS and discovery of ATL has prompted the use of FCM in clinical assessment. In the case of AIDS, detection or quantification of CD8 positive cells in peripheral lymphocytes has provided useful information to some extent. Further, in the case of ATL, detection or quantification of CD3 and/or CD4 positive cells in peripheral lymphocytes has provided some information. This idea has lead to conventional FCM detection in the field of hematopoietic tumor cell analysis. This detection, so-called “lymphocyte subset detection”, determines whether a target cell is positive or negative in comparison to control, and has allowed extremely clear analyses, however, as a result, no useful information has been obtained that is critical to therapeutic guidance.

Next, FCM has been applied to hematopoietic tumor cells. There is advantage in that a large amount of cells may be assessed in shorter period of time than is the case where human being conducts gross-inspection by microscopy. However, in these methods, the methods of analyses have not been varied, information obtained as a result, has only been used as supplementary data with respect to borderline cases when determining morphologically by conventional methods. It has been recognized that a specific method is necessary for the analysis of hematopoietic tumor cells. A variety of methods have been examined for application to hematopoietic tumor cells, however, conventional methods have only focused on finding a marker specific to tumor cells. Amongst all of the above methods, some researchers have reported detection of minor tumor cells using a unique method, which appears to be more useful in clinical settings than conventional methods. However, major problems have been associated with focusing only on tumor cells. If it is reported to a medical practitioner that “there are remaining tumor cells”, he will conduct therapy. However, bone marrow is a collection of a variety of blood cells. Without considering the dynamics of other cells, treatment is conducted focusing on only the tumor cells only, and then the other cells, which have been reconstructed in a normal manner, will be depleted. Accordingly, the present invention provides therapies allowing survival of other cells, which have been regenerated, in an embodiment.

Hereinafter preferable embodiments of the invention will be described. It should be appreciated that those skilled in the art will readily carry out other embodiments thereof from the disclosure of the present application in view of common general knowledge of the art and well known and routine technology of the art, and thus will understand the actions and effects attained by the present invention.

### EFFECTS OF THE INVENTION

Accordingly, the present invention provides therapy in which other cells regenerated from transplanted cells in leukemia therapies and the like.

### BRIEF DESCRIPTION OF DRAWINGS

FIG. 1A depicts a representative scheme of the differentiation of a hematopoietic stem cell.

FIG. 1B depicts identification of the monocytic lineage Stage 1.

FIG. 1C depicts identification of the monocytic lineage Stage 2.

FIG. 1D depicts identification of the monocytic lineage Stage 3.

FIG. 1E depicts identification of the myeloid lineage Stage 1.

FIG. 1F depicts identification of the myeloid lineage Stage 2.

FIG. 1G depicts identification of the myeloid lineage Stage 3.

FIG. 1H depicts identification of the myeloid lineage Stage 4.

FIG. 1I depicts identification of the myeloid lineage Stage 5.

FIG. 1J depicts identification of the B lymphocytic lineage Stage 1.

FIG. 1K depicts identification of the B lymphocytic lineage Stage 2.

FIG. 1L depicts identification of the B lymphocytic lineage Stage 3.

FIG. 1M depicts identification of the B lymphocytic lineage Stage 4.

FIG. 1N depicts identification of the T lymphocytic lineage Stage 1.

FIG. 1O depicts identification of the T lymphocytic lineage Stage 2.

FIG. 1P depicts identification of the T lymphocytic lineage Stage 3.

FIG. 1Q depicts identification of the T lymphocytic lineage Stage 4.

FIG. 1R is a schematic showing the differentiation/maturation stages when a hematopoietic stem cell differentiates into a variety of different cells.

FIG. 1R-A depicts the normal pathway of bone marrow cells. Comparative figures comparing the conventional normal pathway and the normal pathway of the present invention are shown with respect to bone marrow cell normal pathways.

FIG. 1R-B depicts the relationship between maturation stage and differentiation markers.

FIG. 1R-C depicts the variation of surface antigens associated with differentiation of lymphocytes.

FIG. 1R-D depicts the relationship between differentiation of myeloid lineage cells, and surface markers.

FIG. 1R-E depicts the myeloid lineage normal pathway.

FIG. 1R-F depicts the monocytic lineage normal pathway.

FIG. 1R-G depicts the B lymphocytic lineage normal pathway.

FIG. 1R-H depicts an exemplary monitoring experiment of bone marrow reconstruction after hematopoietic stem cell transplantation.

FIG. 1R-I1 depicts an example of the bone marrow normal pathway.

FIG. 1R-I2 depicts another example of the bone marrow normal pathway.

FIG. 1R-I3 depicts an example of the monocyte normal pathway.

FIG. 1R-I4 depicts another example of the monocyte normal pathway.

FIG. 1R-I5 depicts an example of the T lymphocyte normal pathway.

FIG. 1R-I6 depicts another example of the B lymphocyte normal pathway.

FIG. 2 depicts the expression levels of a variety of CD antigens in a variety of cells in differentiation/maturation stages.

FIG. 3 depicts an MDS cell analysis using normal pathway panel.

FIG. 4 depicts an analysis of cells in bone marrow blood using a normal pathway panel.

FIG. 5 depicts Stage-2 Mono (CD34×CD4×CD45×CD10) in patient Ja-0095.

FIG. 6 depicts Stage-2 Mono (CD14×CD33×CD45×CD11b) in patient Ja-0095.

FIG. 7 depicts Stage-1 and Stage-2 B-Lymph (CD19×CD10×CD45×CD34) in patient Ja-0096.

FIG. 8 depicts Stage-2 Myelo (HLA-DR×CD10×CD11b) in patient Ja-0096.

FIG. 9 depicts Stage 4 Myelo (HLA-DR×CD10×CD45×CD11b) in patient Ja-0096.

FIG. 10 depicts Stage-1 and Stage-2 B-Lymph (CD19×CD10×CD45×CD34) in patient Ja-0099.

FIG. 11 depicts Stage-2 Myelo (HLA-DR×CD10×CD11b) in patient Ja-0099.

### BEST MODE FOR CARRYING OUT THE INVENTION

Hereinafter, the present invention will be described optionally by way of illustrative examples with reference to the accompanying drawings. The present invention will be described below. It should be understood throughout the present specification that articles for singular forms include the concept of their plurality unless otherwise mentioned. Also, it should be also understood that terms as used herein have definitions ordinarily used in the art unless otherwise mentioned. Therefore, all technical and scientific terms used herein have the same meanings as commonly understood by those skilled in the relevant art. Otherwise, the present application (including definitions) takes precedence.

The following embodiments are provided for a better understanding of the present invention and the scope of the present invention should not be limited to the following description. It will be clearly appreciated by those skilled in the art that variations and modifications can be made without departing from the scope of the present invention with reference to the specification.

### DEFINITION

Terms Specifically Used Herein Will be defined below.

The term “cell” is herein used in its broadest sense in the art, referring to a structural unit of tissue of a multicellular organism, which is capable of self replicating, has genetic information and a mechanism for expressing it, and is surrounded by a membrane structure which isolates the living body from the outside. Cells used herein may be either naturally-occurring cells or artificially modified cells (e.g., fusion cells, genetically modified cells, etc.). Examples of cell sources include, but are not limited to, a single-cell culture; the embryo, blood, or body tissue of normally-grown transgenic animal; a cell mixture of cells derived from normally-grown cell lines; and the like.

As used herein, the term “stem cell” refers to a cell having self-replication ability and pluripotency, and in actual situations, a cell capable of regenerating into a tissue, which has been injured, to some extent. A stem cell for use in the present invention may be an embryonic stem (ES) cell or tissue stem cell (also called tissue specific stem cell or somatic stem cell). Embryonic stem cells are pluripotent stem cells derived from early embryos. An embryonic stem cell was first established in 1981, which has been applied to the production of knockout mice since 1989. In 1998, a human embryonic stem cell was established, which is currently becoming available for regenerative medicine.

Tissue stem cells have a relatively limited level of differentiation unlike embryonic stem cells. Tissue stem cells are present in tissues and have an undifferentiated intracellular structure. Tissue stem cells have a higher nucleus/cytoplasm ratio and have few intracellular organelles. Most tissue stem cells have pluripotency, a long cell cycle, and proliferative ability beyond the life of the individual.

Tissue stem cells may be classified into three categories in accordance with their origins: ectoderm, mesoderm, and endoderm. Ectoderm-derived tissue stem cells include neural stem cells existing in the brain, epidermal stem cells existing in the skin, follicular stem cells and pigment stem cells. Mesoderm-derived tissue stem cells include vascular stem cells, hematopoietic stem cells and mesenchymal stem cells observed in bone marrow and blood. Endoderm-derived tissue stem cells are mainly present in organs, including liver stem cells, pancreatic stem cells, and intestinal epithelial stem cells.

Tissue stem cells are separated into categories of sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

As used herein, the term “somatic cell” refers to any cell other than a germ cell, such as an egg, a sperm, or the like, which does not transfer its DNA to the next generation. Typically, somatic cells have limited or no pluripotency. Somatic cells used herein may be naturally-occurring or genetically modified as long as they can achieve the intended treatment.

The origin of a cell is categorized as being ectoderm, endoderm, or mesoderm. Cells of ectodermal origin are mostly present in the brain, including neural stem cells. Cells of endodermal origin are mostly present in bone marrow, including blood vessel stem cells, hematopoietic stem cells, mesenchymal stem cells, and the like. Cells of mesoderm origin are mostly present in organs, including liver stem cells, pancreas stem cells, and the like. Somatic cells may be herein derived from any germ layer. Preferably, somatic cells, such as lymphocytes, spleen cells or testis-derived cells, may be used.

As used herein, the term “isolated” means that naturally accompanying material is at least reduced, or preferably substantially completely eliminated, in normal circumstances. Therefore, the term “isolated cell” refers to a cell substantially free from other accompanying substances (e.g., other cells, proteins, nucleic acids, etc.) in natural circumstances. The term “isolated” in relation to nucleic acids or polypeptides means that, for example, the nucleic acids or the polypeptides are substantially free from cellular substances or culture media when they are produced by recombinant DNA techniques; or precursory chemical substances or other chemical substances when they are chemically synthesized. Isolated nucleic acids are preferably free from sequences naturally flanking the nucleic acid within an organism from which the nucleic acid is derived (i.e., sequences positioned at the 5′ terminus and the 3′ terminus of the nucleic acid).

As used herein, the term “established” in relation to cells refers to the state of a cell in which a particular property (pluripotency) of the cell is maintained and the cell undergoes stable proliferation under culture conditions. Therefore, established stem cells maintain pluripotency. In the present invention, the use of established stem cells is preferable since the step of collecting stem cells from a host can be avoided.

As used herein, the term “pathway”, which is used for differentiation/maturation of a cell, refers to a profile of the expression level of cellular markers which vary depending on the differentiation or maturation stages

As used herein, the term “normal Pathway”, which is used for differentiation/maturation of a cell, refers to a profile of the expression level of cellular markers when a normal differentiation/maturation occurs in the cell of interest. Such a profile can be expressed by the use of flow cytometry.

As used herein, the term “differentiated cell” refers to a cell having a specialized function and form (e.g., muscle cells, neurons, etc.). Unlike stem cells, differentiated cells have little or no pluripotency. Examples of differentiated cells include epidermal cells, pancreatic parenchymal cells, pancreatic duct cells, hepatic cells, blood cells, cardiac muscle cells, skeletal muscle cells, osteoblasts, skeletal myoblasts, neurons, vascular endothelial cells, pigment cells, smooth muscle cells, fat cells, bone cells, cartilage cells, and the like.

As used herein, the terms “differentiation” or “cell differentiation” refers to the phenomenon wherein two or more types of cells having qualitative differences in form and/or function occur in a daughter cell population derived from the division of a single cell. Therefore, “differentiation” includes a process during which a population (family tree) of cells, which do not originally have a specific detectable feature, acquire a feature, such as the production of a specific protein, or the like. At present, cell differentiation is generally considered to be a state of a cell in which a specific group of genes in the genome are expressed. Cell differentiation can be identified by searching for intracellular or extracellular agents or conditions which elicit the above-described state of gene expression. Differentiated cells are stable in principle. Particularly, animal cells which have been differentiated once rarely differentiate into other types of cells.

As used herein, the term “pluripotency” refers to a nature of a cell, i.e., an ability to differentiate into one or more, preferably two or more, tissues or organs. Typically, the pluripotency of a cell is limited as the cell is developed, and in an adult, cells constituting a tissue or organ rarely alter to different cells, where the pluripotency is lost. Such alteration typically occurs in pathological conditions, and is called metaplasia. However, mesenchymal cells tend to easily undergo metaplasia, i.e., alter to other mesenchymal cells, with relatively simple stimuli. Therefore, mesenchymal cells have a high level of pluripotency. Embryonic stem cells have pluripotency or totipotency. Tissue stem cells have pluripotency with limitation specific to the types thereof.

As used herein, one type of pluripotency is “totipotency”, which refers to an ability to be differentiated into all types of cells which constitute an organism. The idea of pluripotency encompasses totipotency. An example of a totipotent cell is a fertilized ovum. Note that totipotency may be clearly separated from pluripotency. The former indicates an ability to be differentiated into any type of cell while the latter indicates an ability to be committed into a plurality of types of cell, but not all types.

As used herein, one type of pluripotency is “totipotency”, which refers to an ability to be differentiated into all types of cell which constitute an organism. The concept of pluripotency encompasses totipotency. An example of a totipotent cell is a fertilized ovum. Note that totipotency may be clearly separated from pluripotency if it is used in a strict manner. It can be determined if a cell has totipotency by observing formation of embryoid body in an ex vivo culture system under conditions of differentiation induction, and the like, however the methods for determination are not limited to these. Further, assays to confirm the presence or absence of pluripotency using a living body, include, but are not limited to: observing formation of teratoma by transplantation into immunodeficent mice, formation of chimeric embryos by injection into blastocysts, transplantation into a living tissue, injection into ascites, and the like.

As used herein, totipotency and pluripotency can be determined based on the number of days which has passed after fertilization. For example, for mice, the borderline by which totipotency is distinguished from pluripotency is around Day 8 after fertilization. Although not wishing to be bound by theory, for mice, cells develop over time after fertilization as follows. On Day 6.5 after fertilization (also represented by E6.5), a primitive streak appears on one side of the epiblast, clarifying the future anteroposterior axis of the embryo. The primitive streak indicates the future posterior end of the embryo, extending across the ectoderm to reach the distal end of the cylinder. The primitive streak is an area in which cell movement takes place. As a result, the future endoderm and mesoderm are formed. By E7.5 a head process appears ahead of the node, in which a notochord, a future endoderm (lower layer) and a neural plate (upper layer) around the notochord, are formed. The node appears around E6.5 and moves backward, so that the axial structure is formed from the head to the tail. By E8.5 the embryo is elongated and a large head lamella, mostly consisting of the anterior neural plate, is formed at the anterior end of the embryo. Segments are formed at a rate of one per 1.5 hours from E8 from the head to the tail. After this stage, cells no longer exhibit totipotency or develop into an individual even if they are brought back to the placenta, except by dedifferentiation. Before this stage, cells have totipotency without any particular treatment. Thus, this stage is a branch point of totipotency. Therefore, it is difficult to establish ES cells from embryos after this point. In other words, it is possible to establish cells, typically called EG (germ cell-derived) cells, from embryos after this point. Also, in this context this point can be said to be a branch point. Therefore, in one aspect, markers of the present invention can be used to determine the presence or absence of totipotency or the validity of an ES cell as a starting material.

As used herein the term “embryonic stem cell” and “ES cell” are interchangeably used to refer to any pluripotent stem cell derived from an early embryo. Usually, embryonic stem cells are said to have totipotency or substantial totipotency. This embryonic stem cell has been introduced into a normal host blastocyst to return the same to “foster” parent uterus to produce a chimera, and found to obtain germ-line chimera having high chimera forming capability (chimeric mice having functional reproductive cells) (A. Bradley, et al.: Nature, 309, 255, 1984). Embryonic stem cell lines are amenable to, under culture, a variety of gene introduction methods (for example calcium phosphate methods, retrovirus vector methods, liposome methods, electroporation methods and the like). Further, a method for selecting a cell with a gene incorporated therein is devised to use genetic homologous recombination to introduce modifications (for example, substitution, deletion and/or addition) aimed at a specific gene to produce a clone with the same. Embryonic stem cell line subjected to such treatment in vitro, retain the ability to differentiate into the reproductive lineage, and thus at present a number of studies are being conducted to investigate the functions of a specific gene at an individual level (M. R. Capecchi: Science, 244, 1288, 1989). A stem cell may be an artificially produced cell (e.g., fusion cells, reprogrammed cells and the like) as long as it has the above-described abilities.

As used herein the term “hematopoietic stem cell” refers to a cell which is a source of cell production in the rejuvenation of hematopoietic tissue, intestinal epithelium tissue and the like. This stem cell is capable of maintaining the self, and differentiating into all types of blood cells. It should be understood that such blood cells include, for example, monocytic lineage stem cells, B lymphocytic lineage stem cells, myeloid lineage stem cells, T lymphocytic lineage stem cells, B lymphocytic lineage stem cells, platelet lineage cells, erythrocyte lineage cells, monocytic lineage cells, and the like.

Hematopoietic cells are produced in the bone marrow, and are differentiated into erythrocytes, platelets, leukocytes and the like, and the cells flow through the peripheral blood. With respect to myeloid lineage cells, the origin thereof is pluripotent stem cells, and then there come hematopoietic stem cells which are committed to hematopoietic cells, which are differentiated into pluripotent precursor cells, and then further differentiated into myeloid precursor cells and lymphocyte lineage precursor cells.

In myeloid lineage, pluripotent stem cells are differentiated to cells called CFU-GEMM. Thereafter, differentiation is advanced from the CFU-GEMM through CFU-GEM to CFU-GM, and then myeloblast, promyelocyte, myelocyte and the like. These are present in the bone marrow, and this flows in the peripheral blood as neutrophils after differentiation. In the next lineage, CFU-GM is differentiated to monocytes, and monoblast, promonocyte, and monocyte. Such a monocyte will be appeared in the peripheral blood. In the third lineage, CFU-GEMM is differentiated to BFU-E cell, and then proerythroblast, erythroblast and erythrocyte. Further, there is the so-called megalocyte lineage, in which CFU-Meg (abbreviation of megakaryocyte) is differentiated into megakaryoblast, megakaryocyte, and platelet.

Leukemia, in such a differentiation process, is caused by an abnormality of pluripotent stem cells. Accordingly, the present invention can be applied to therapies of leukemia in the sense of improving such abnormalities.

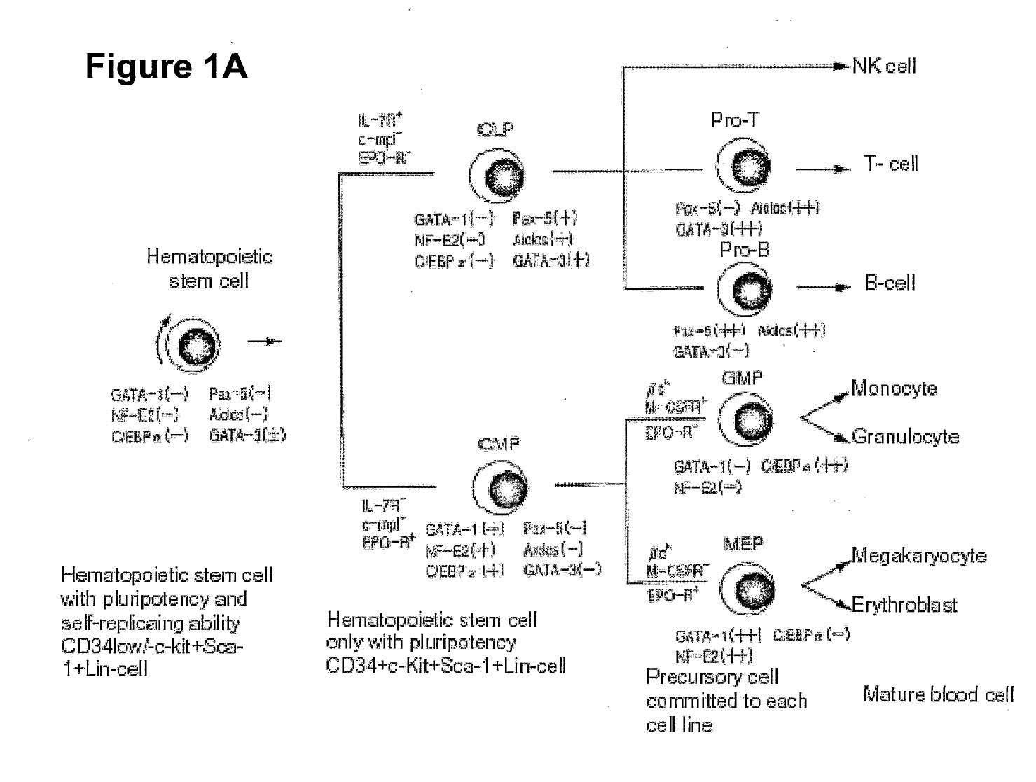

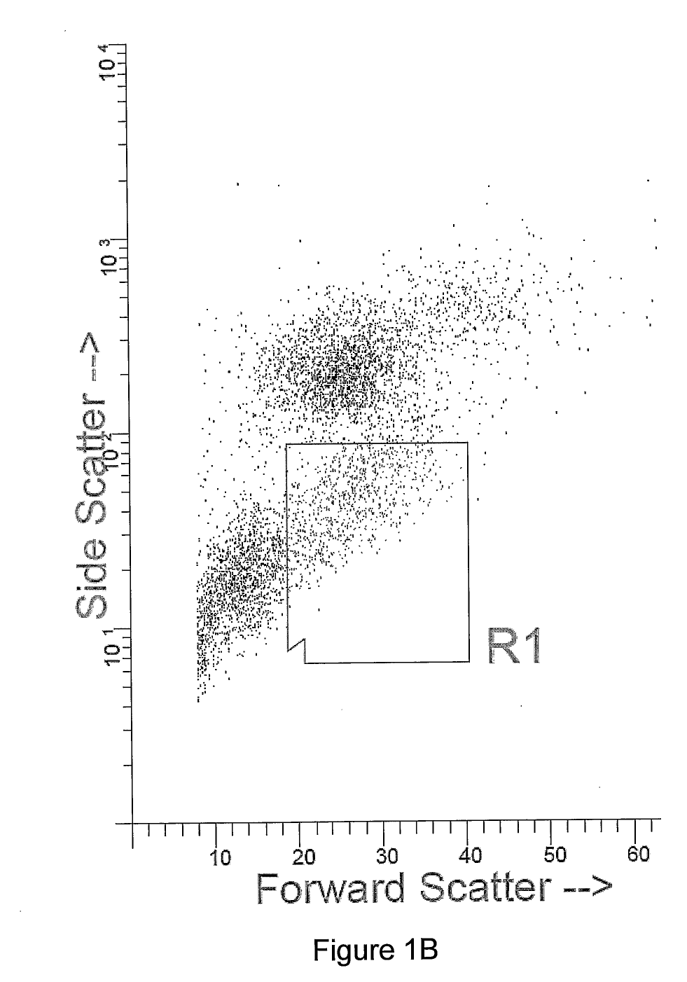

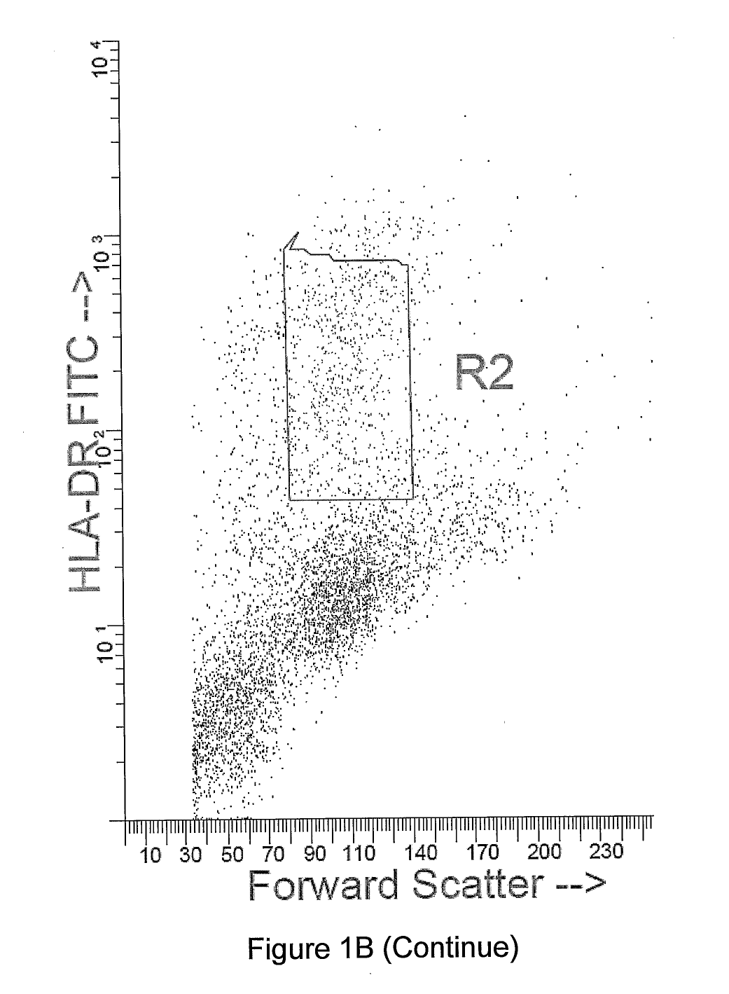

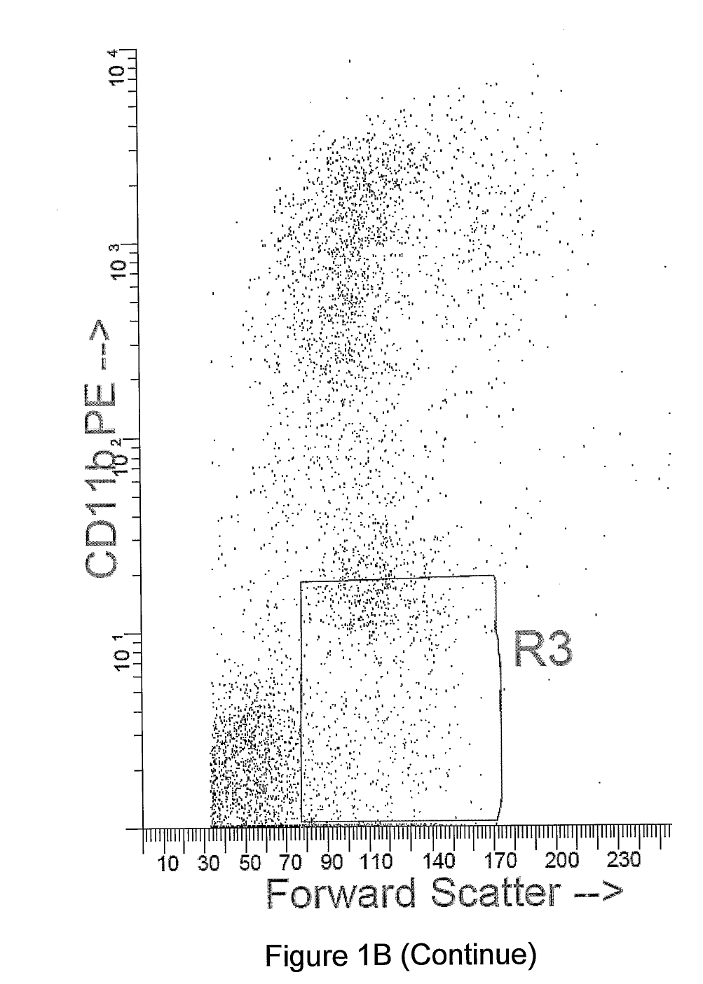

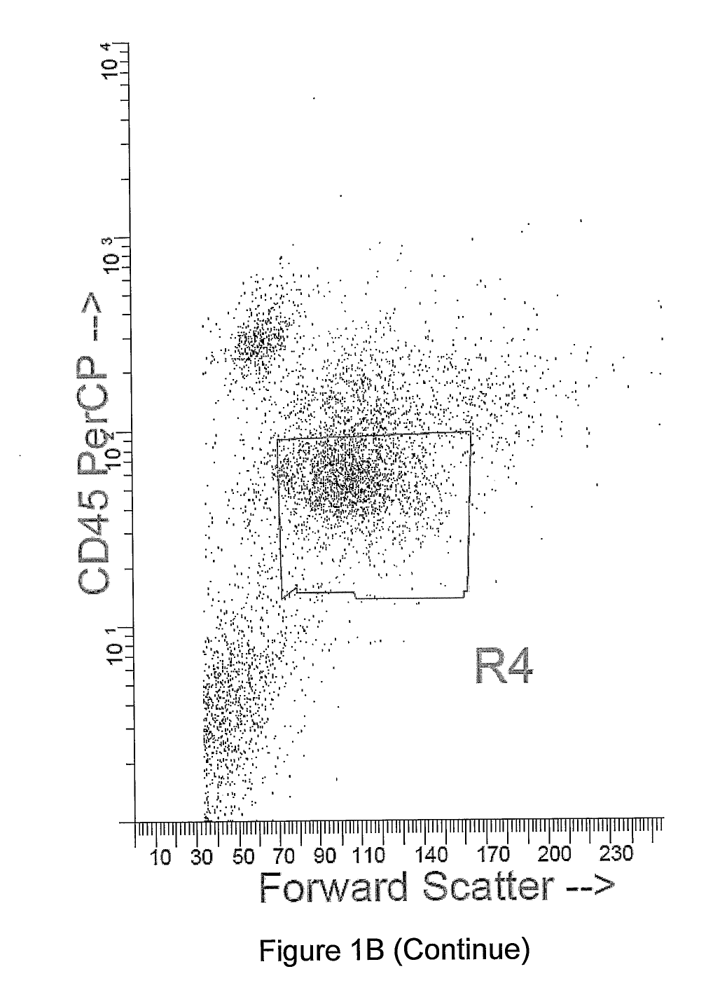

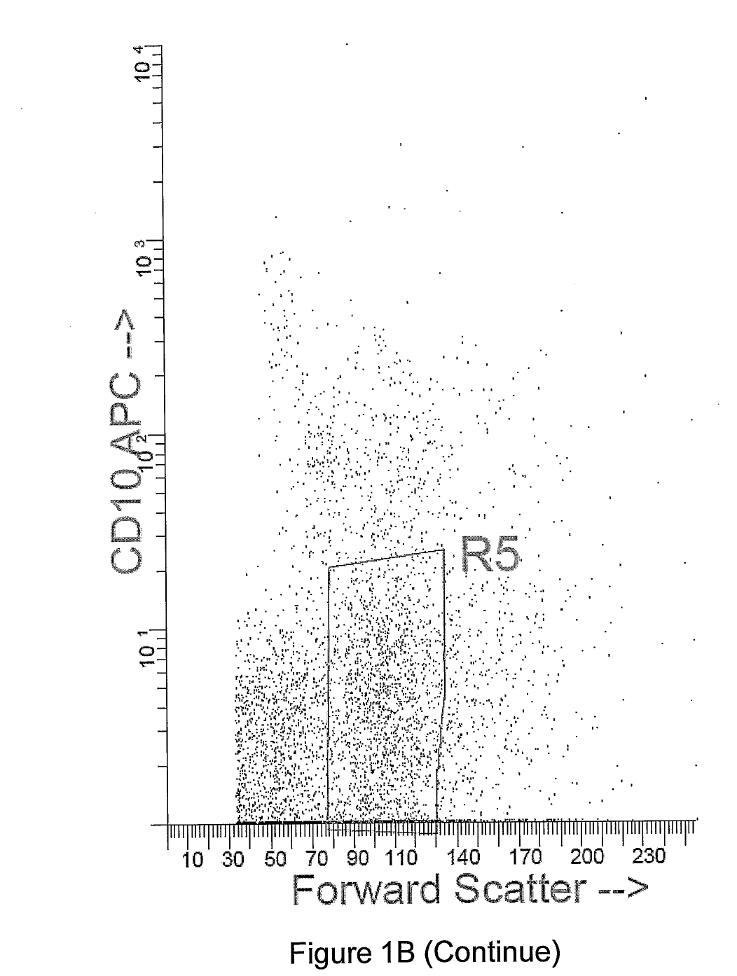

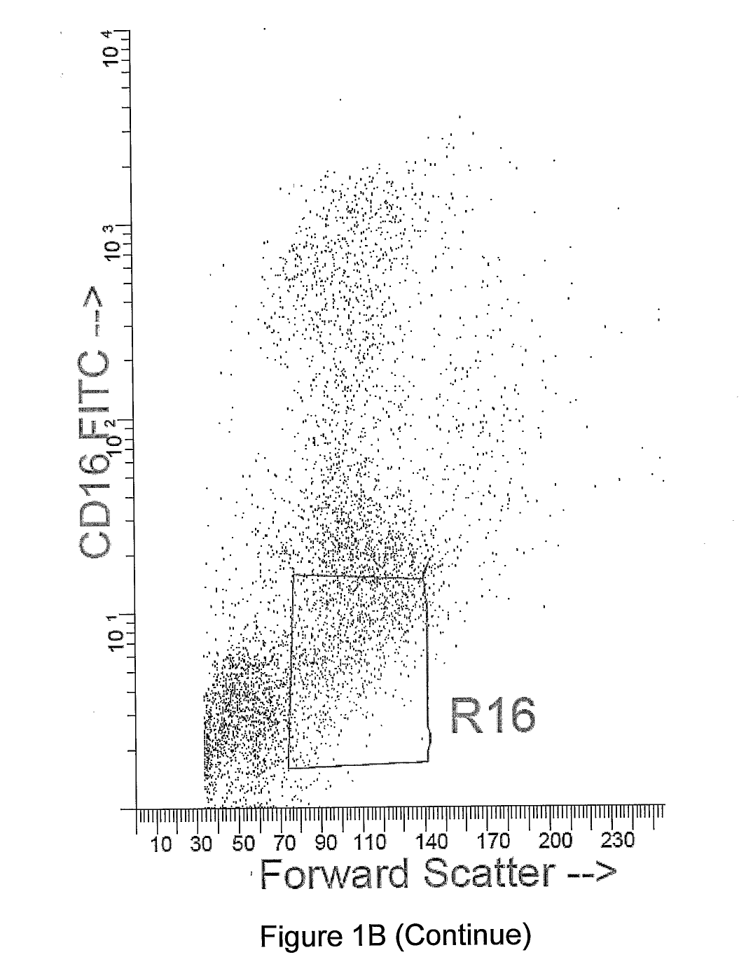

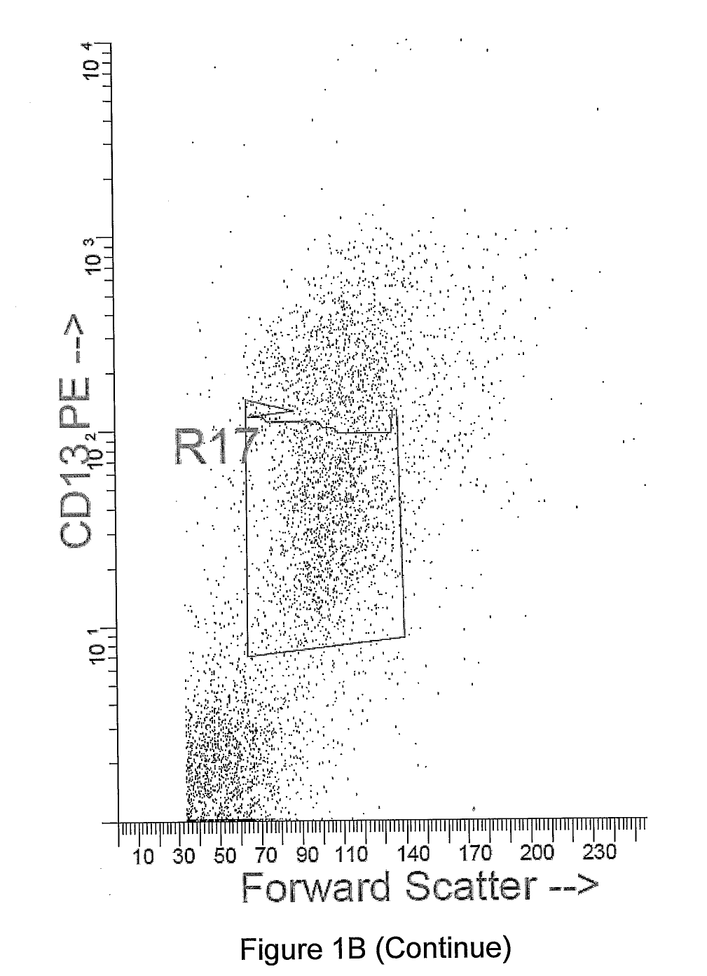

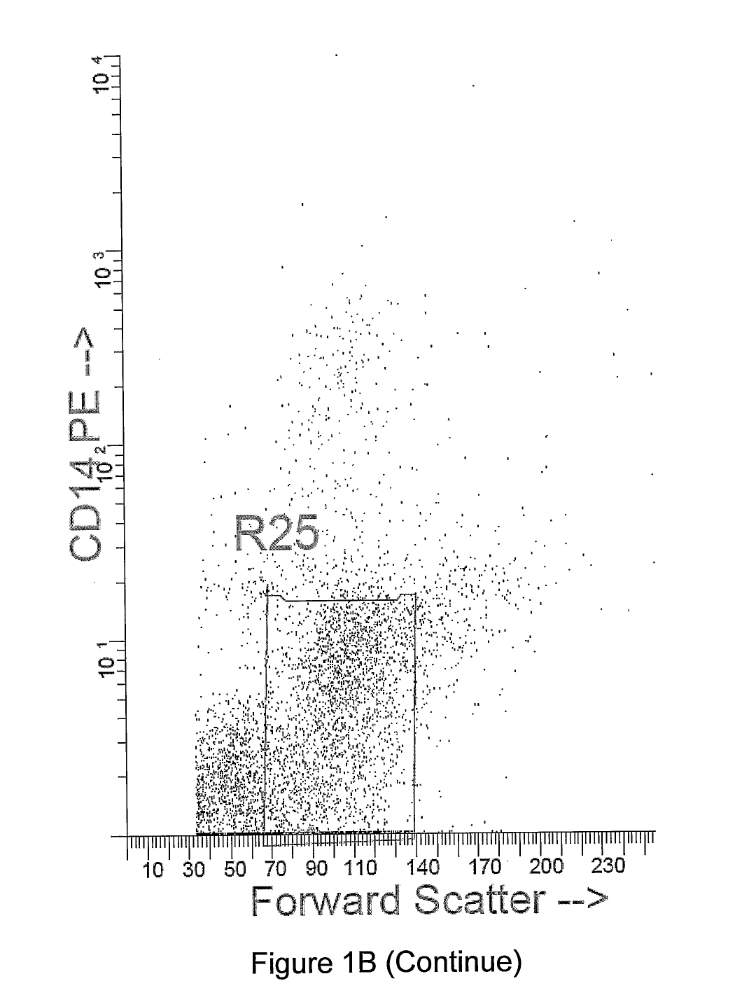

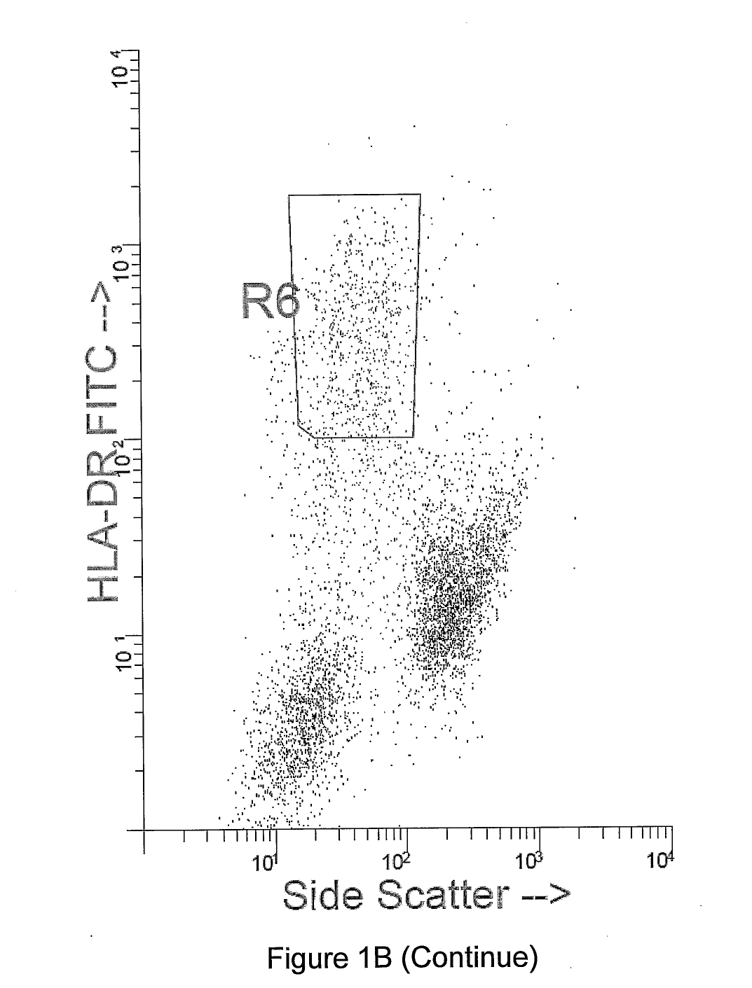

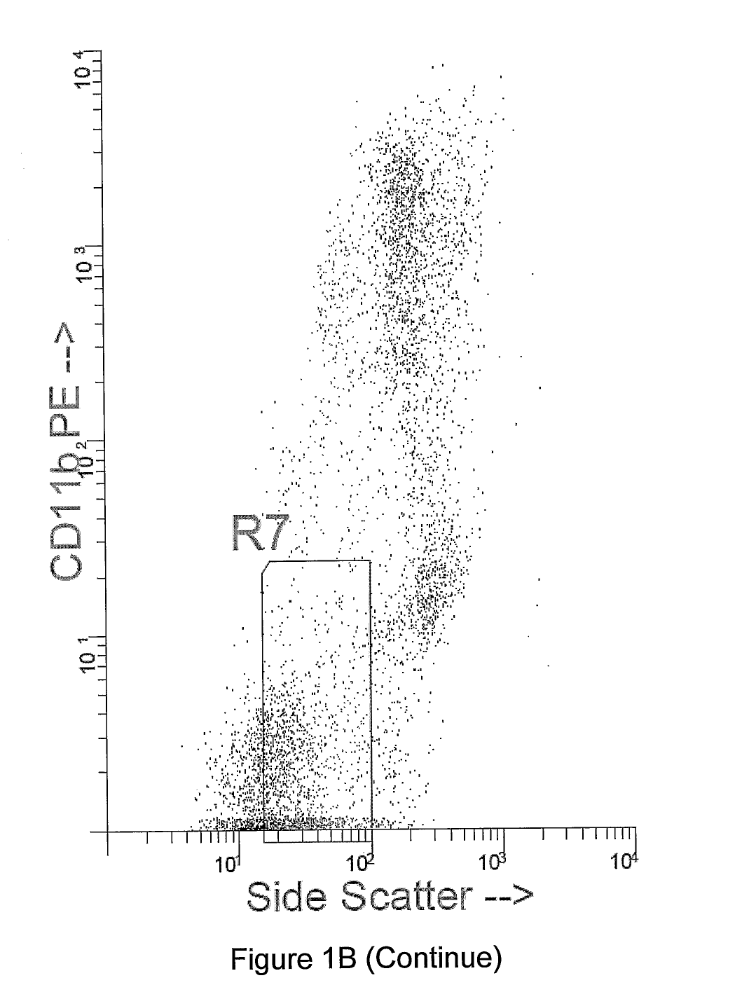

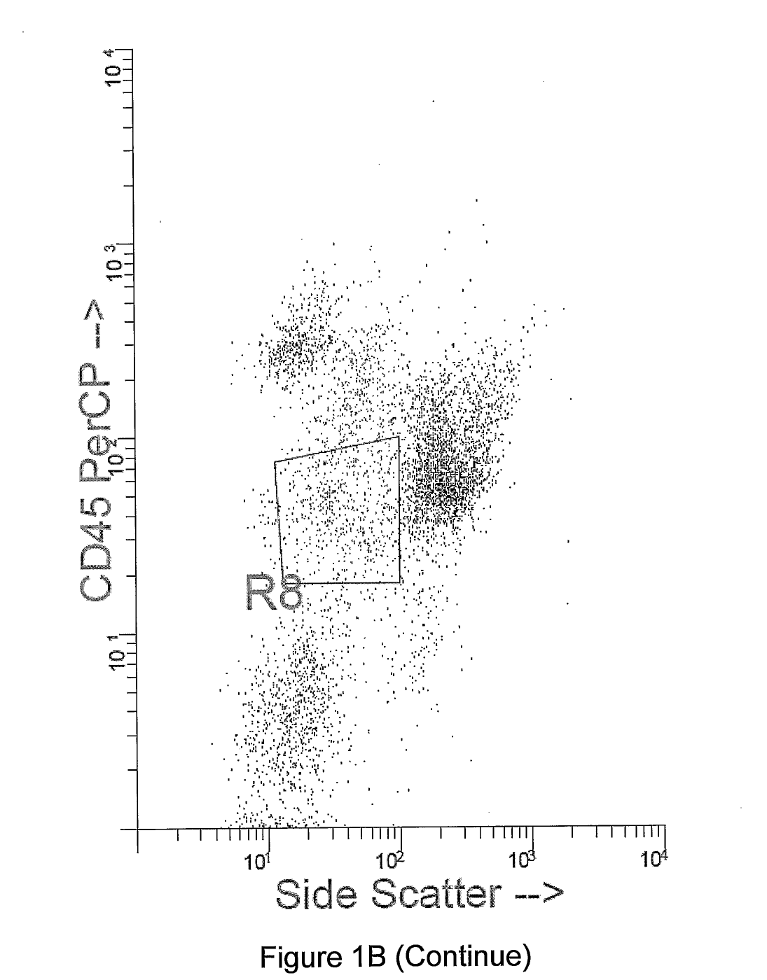

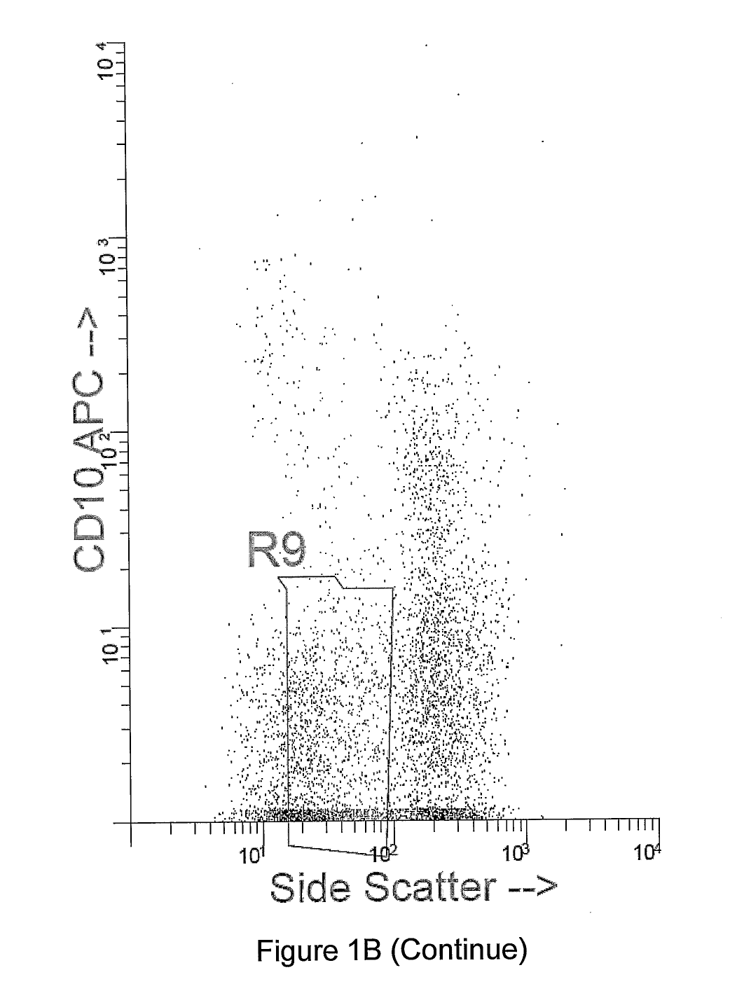

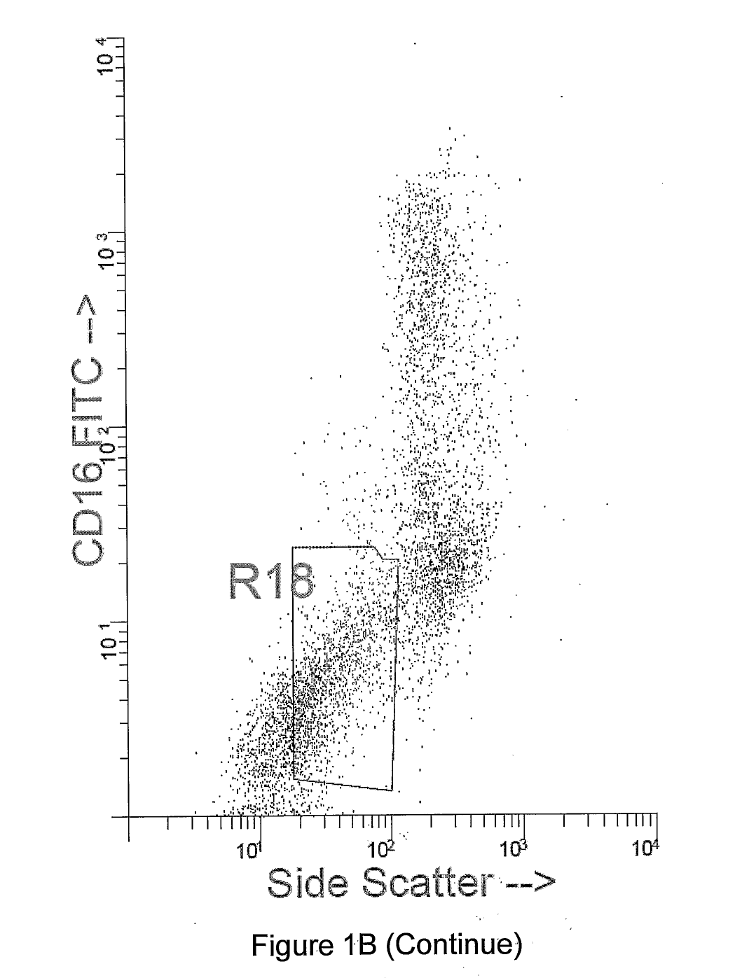

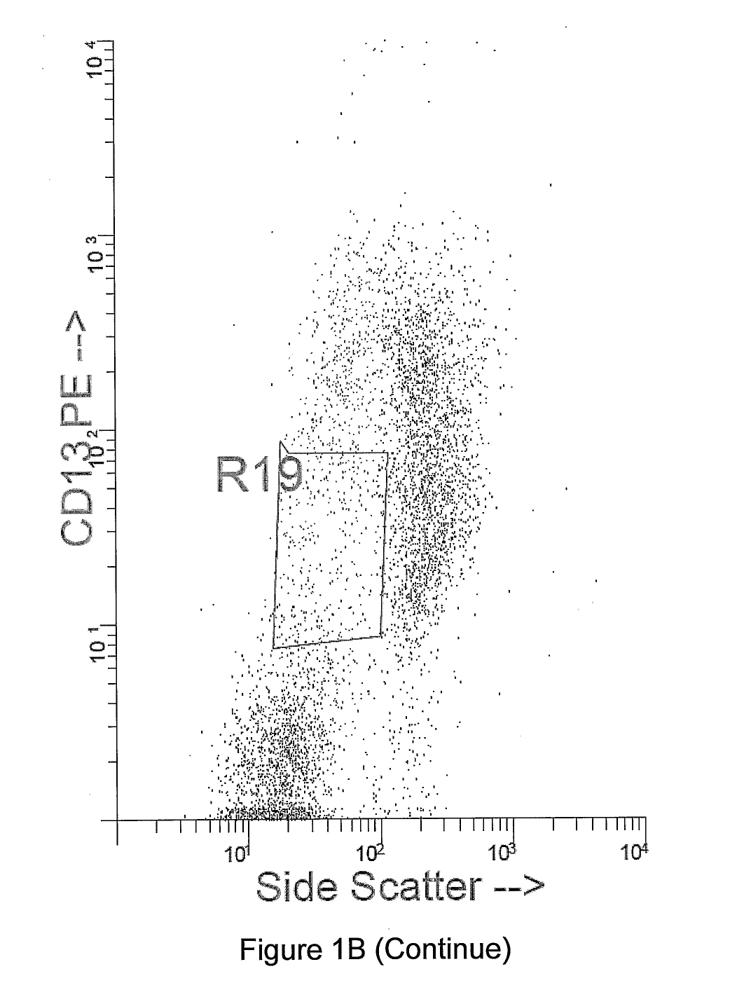

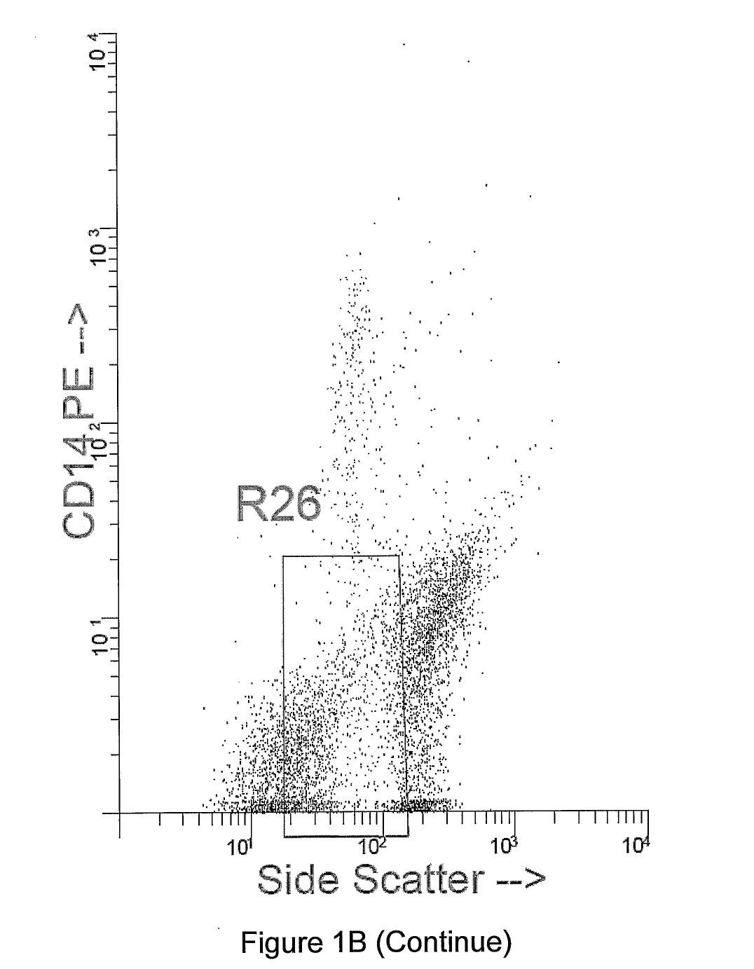

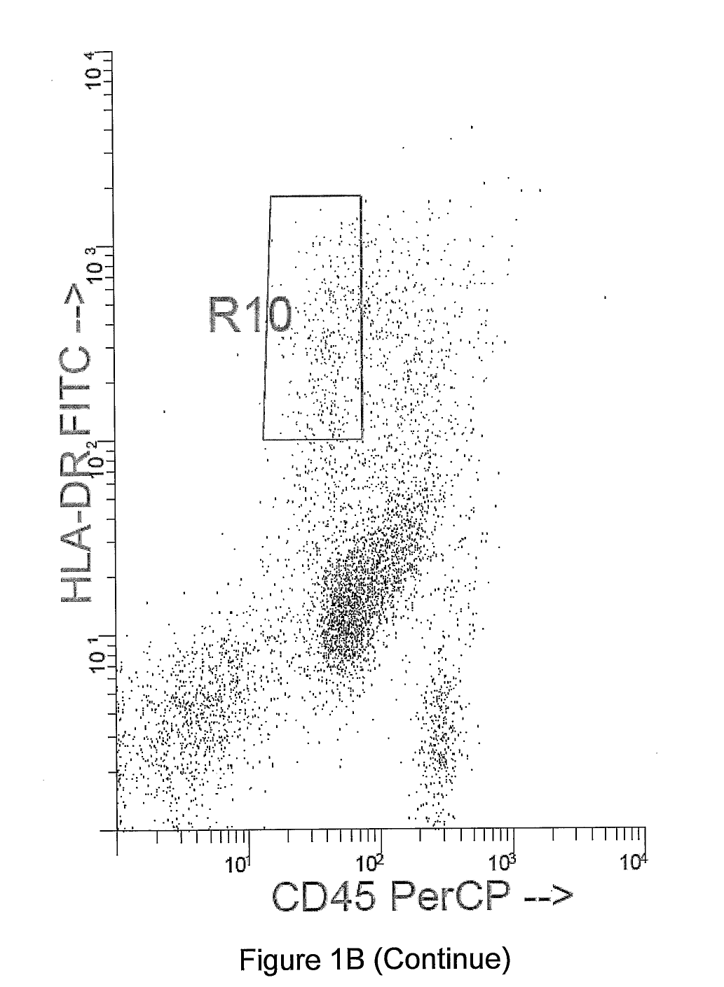

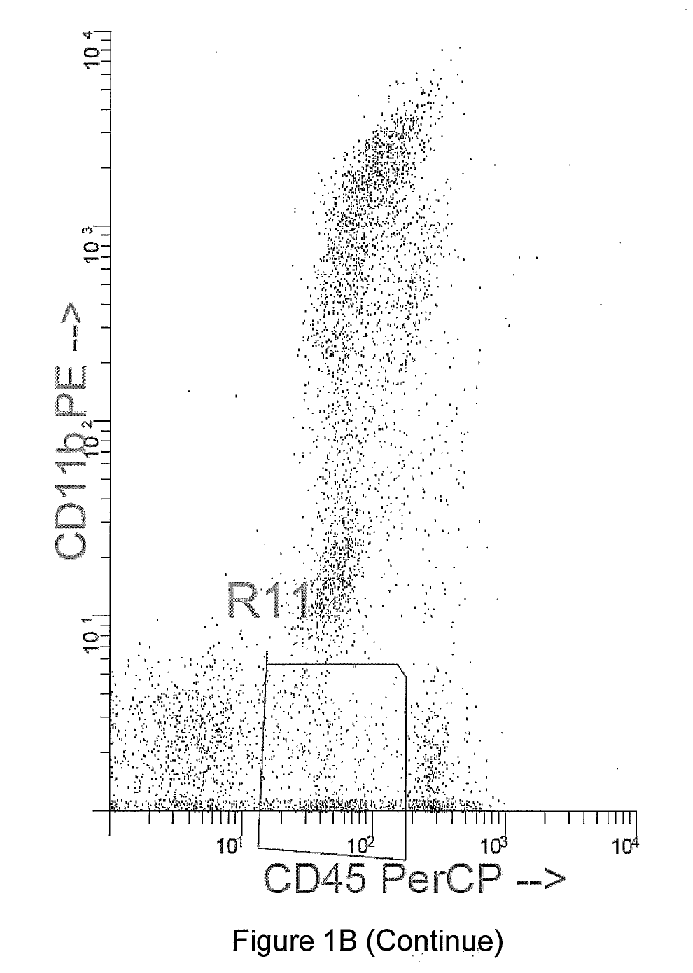

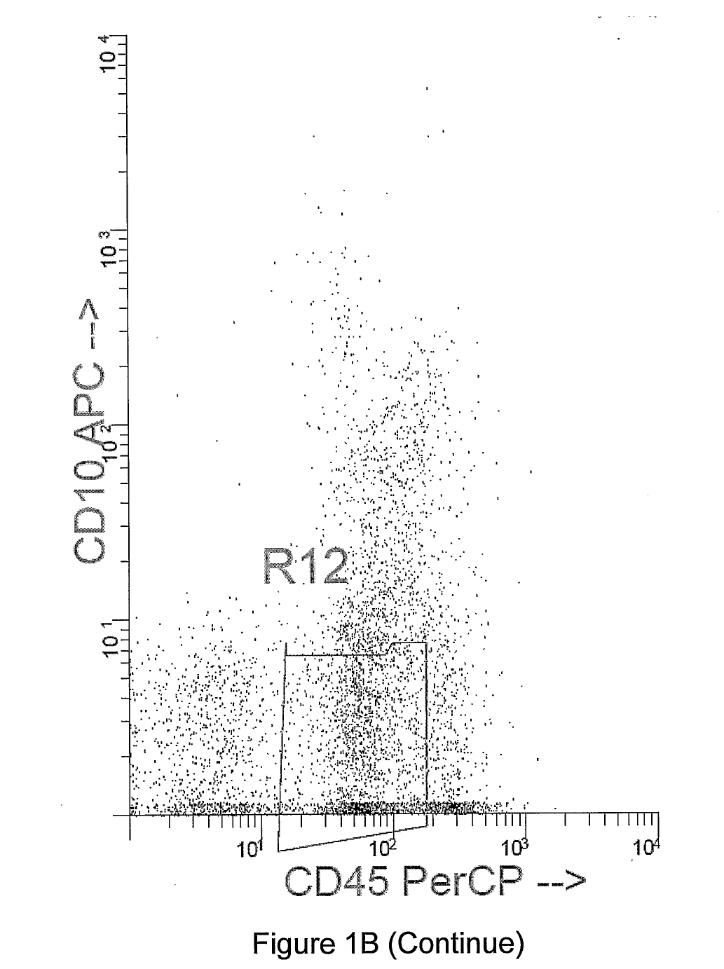

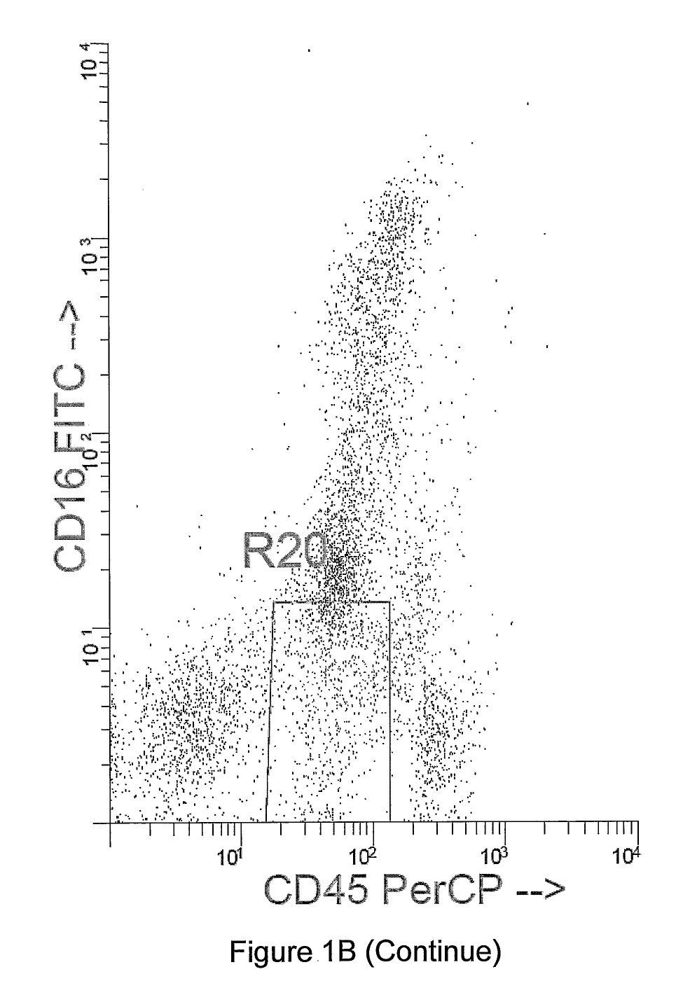

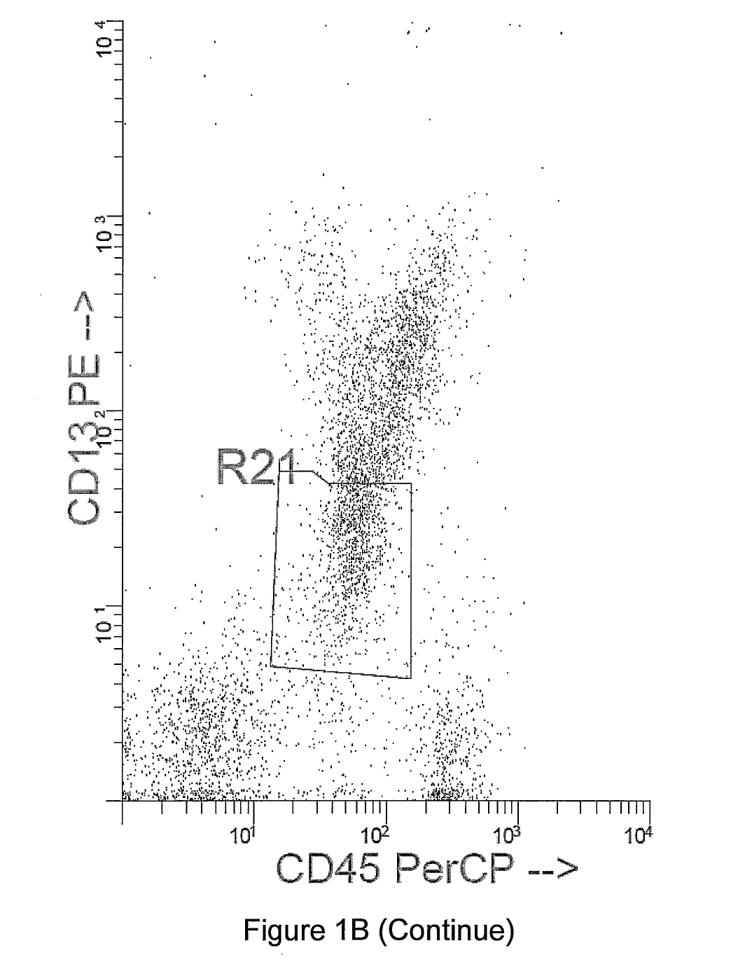

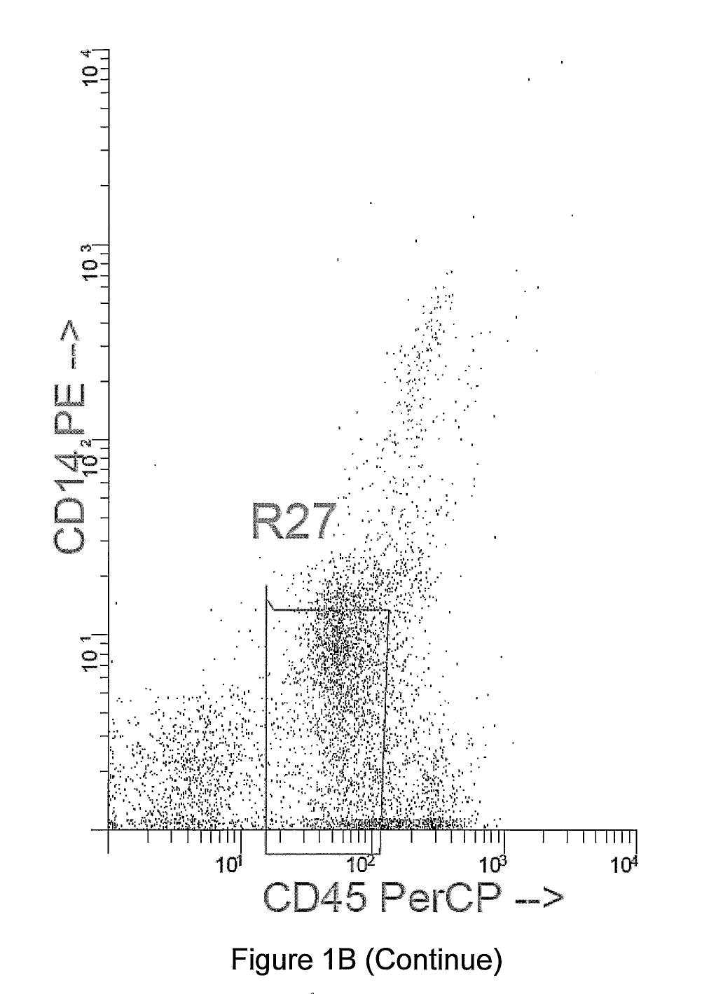

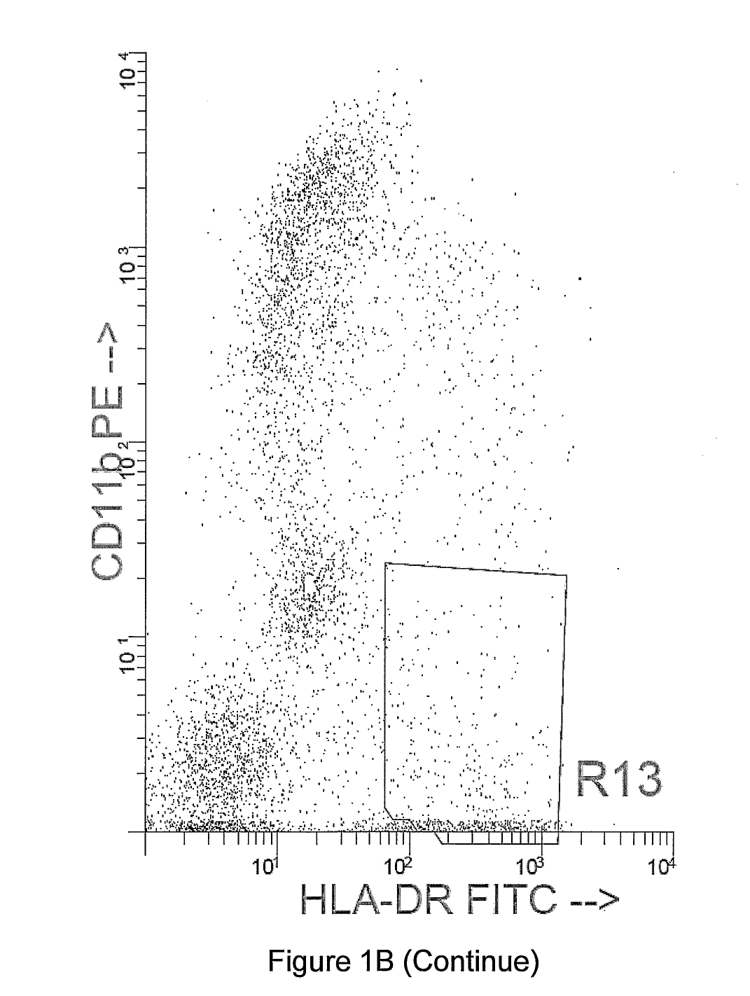

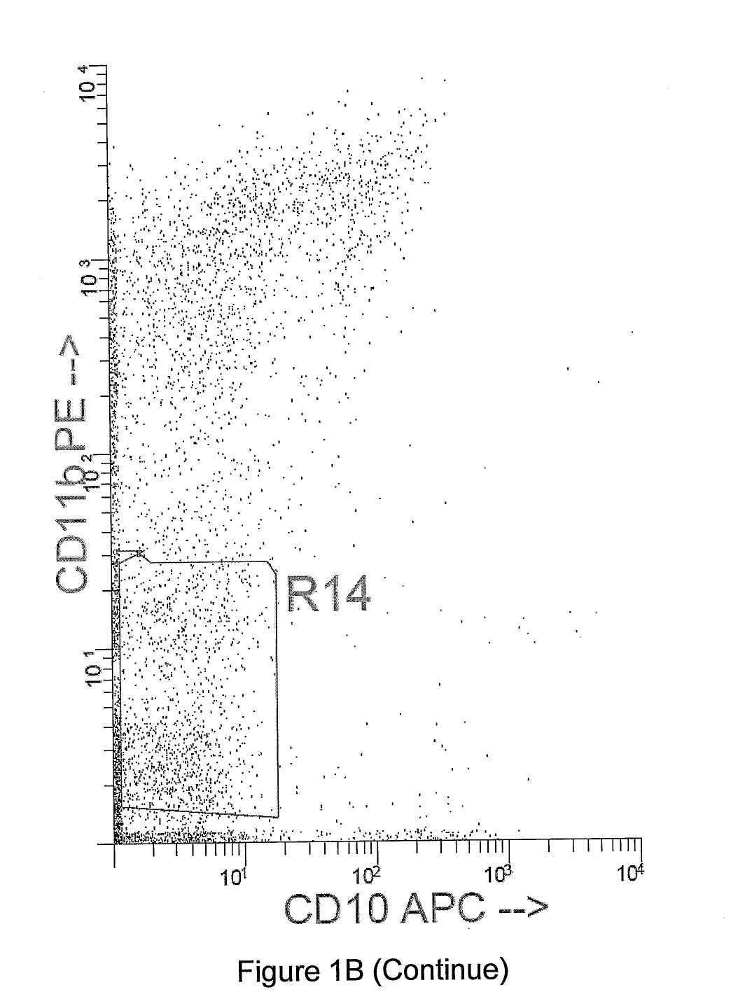

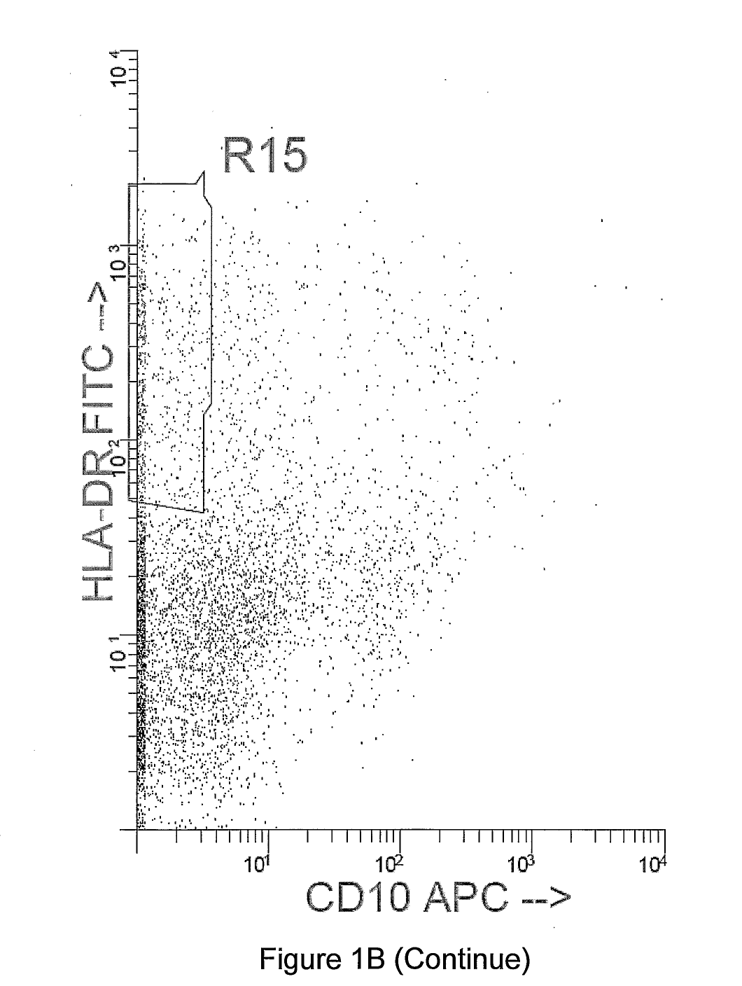

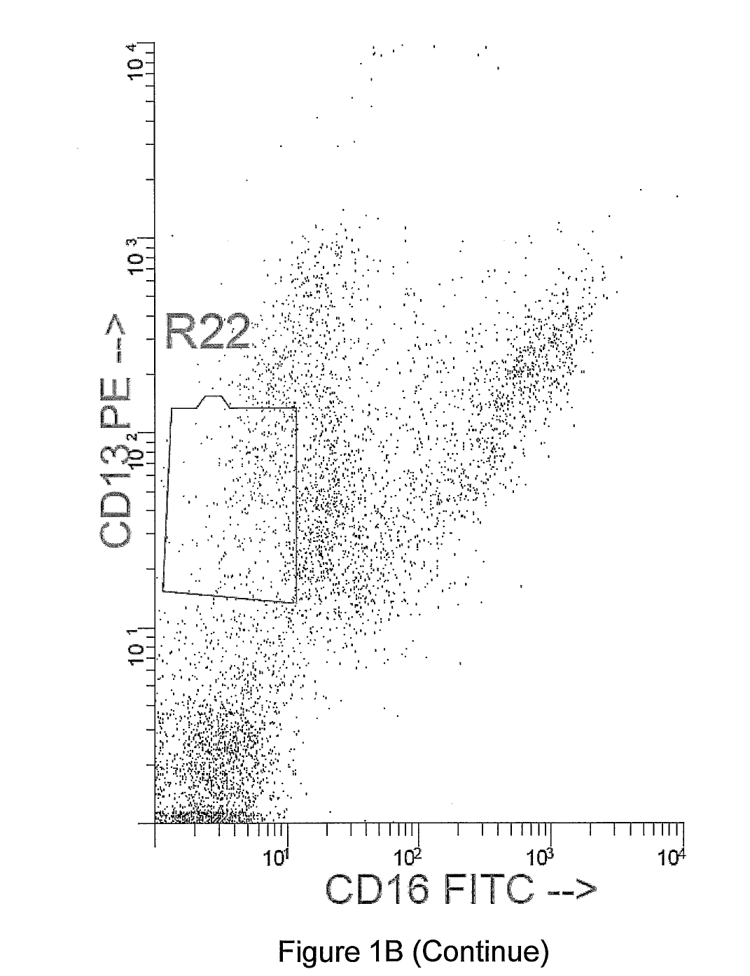

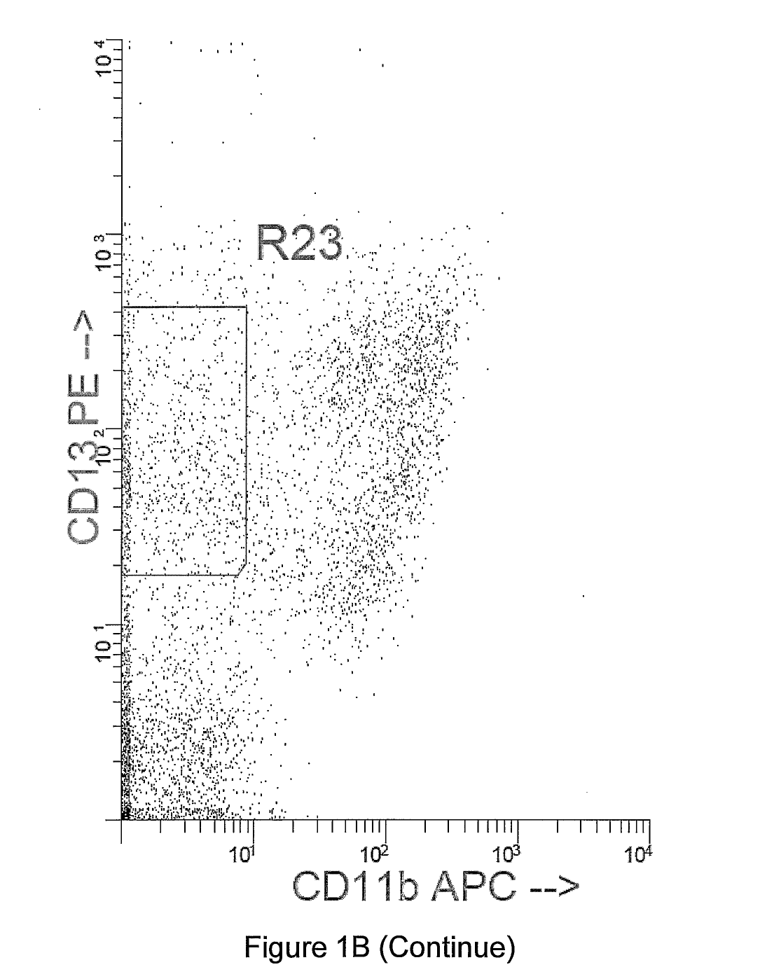

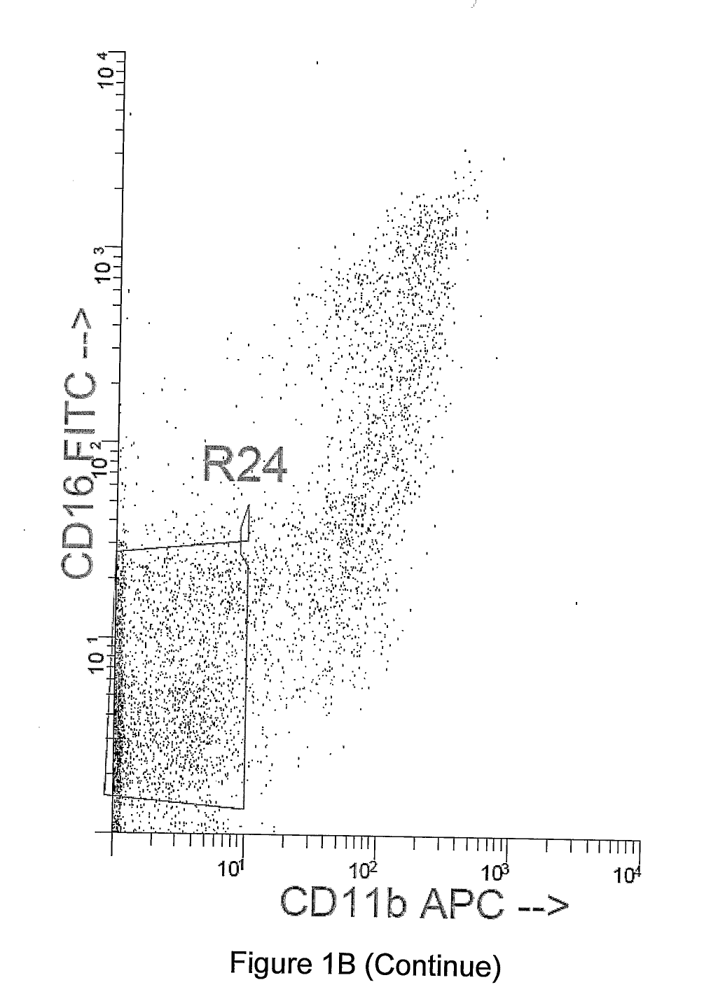

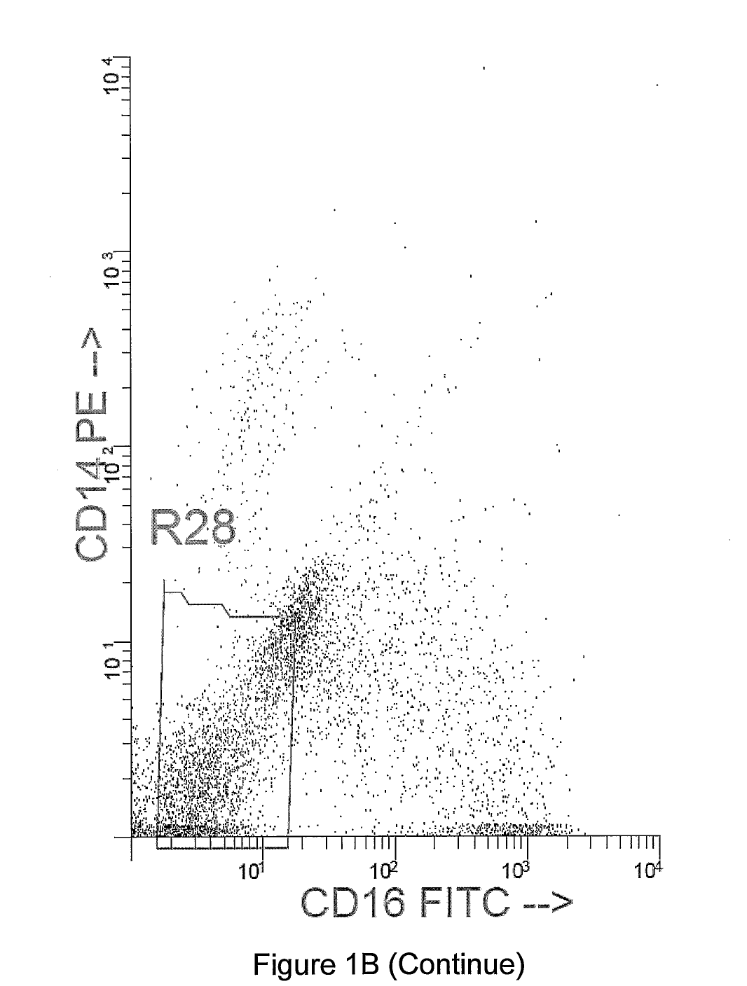

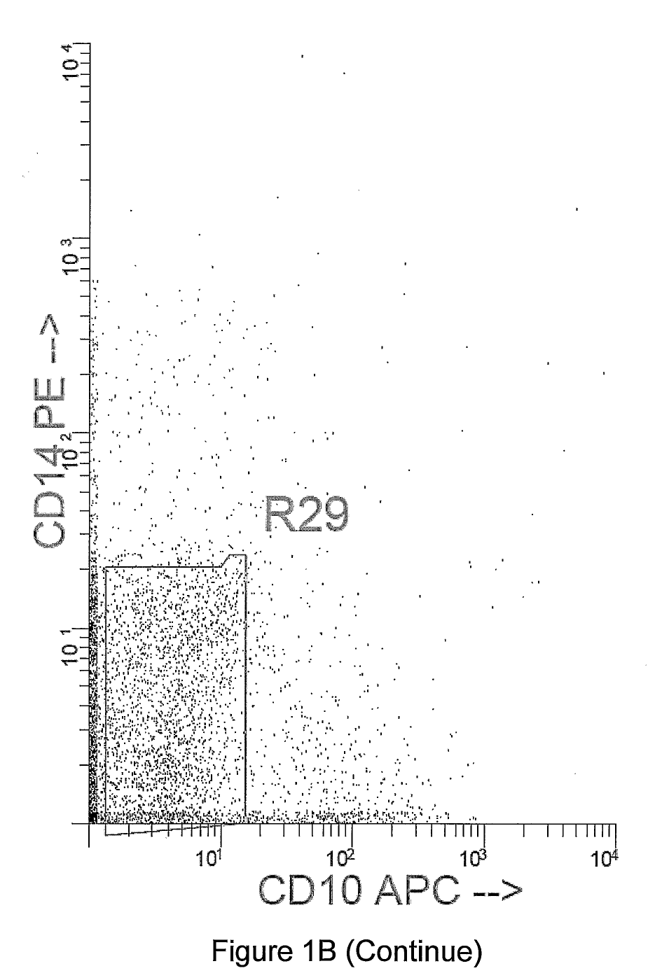

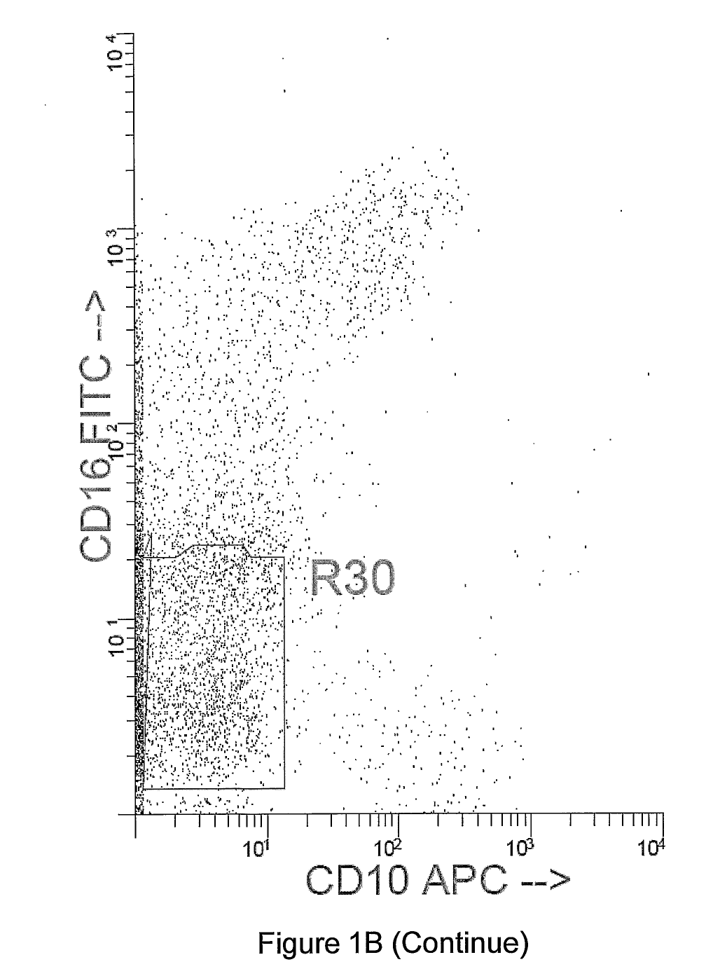

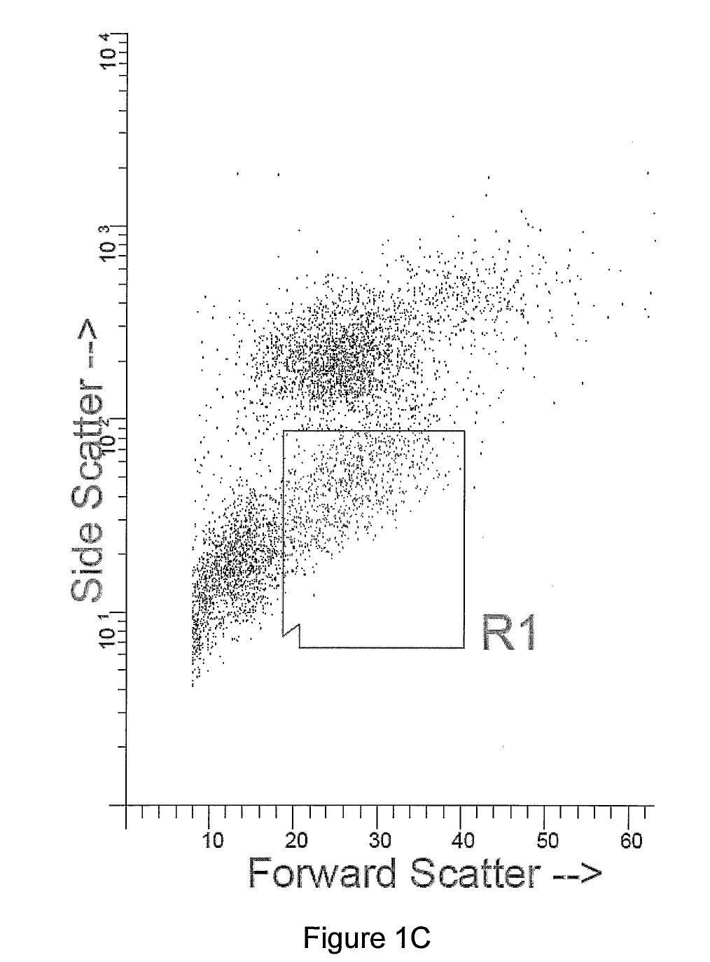

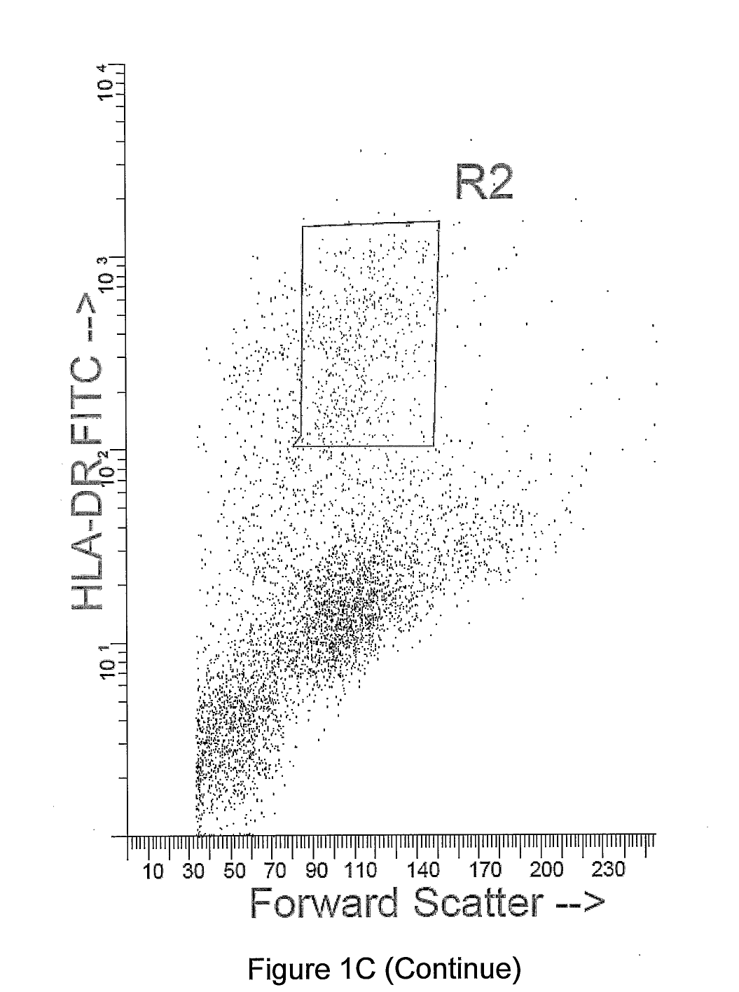

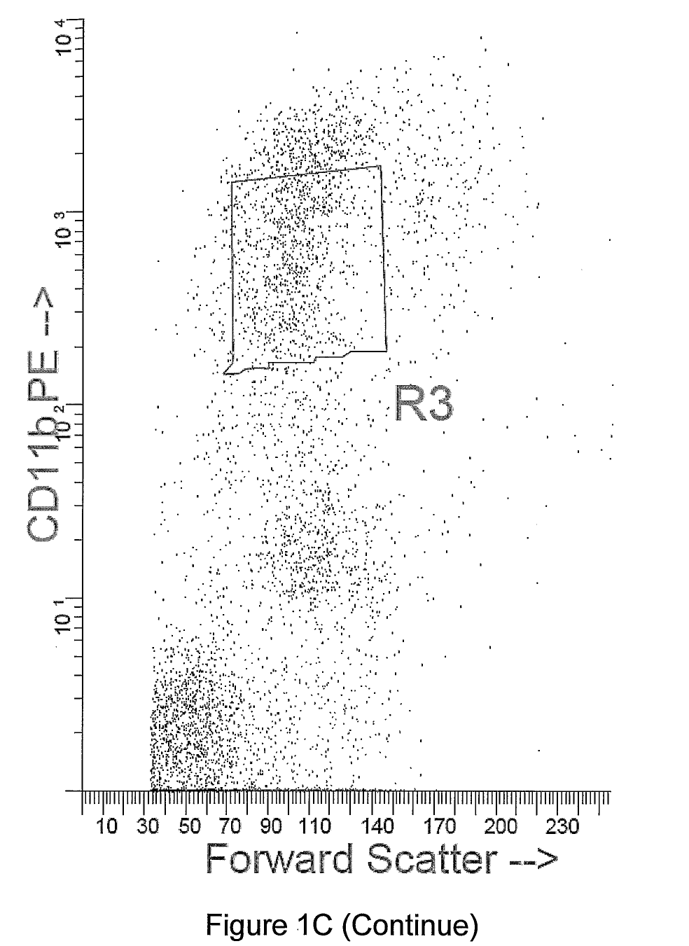

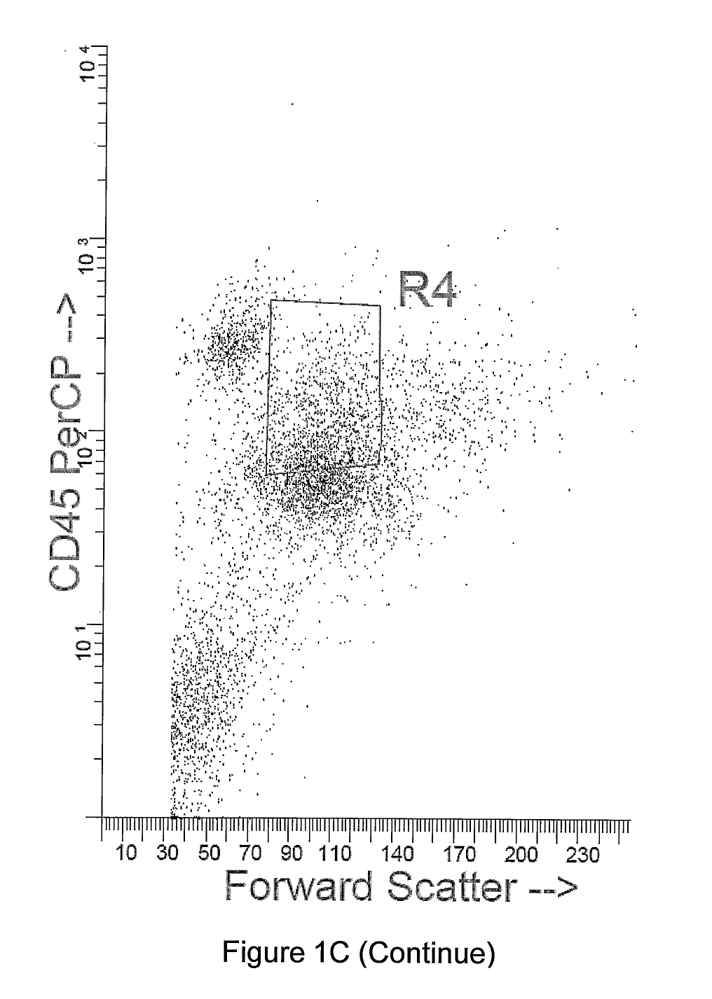

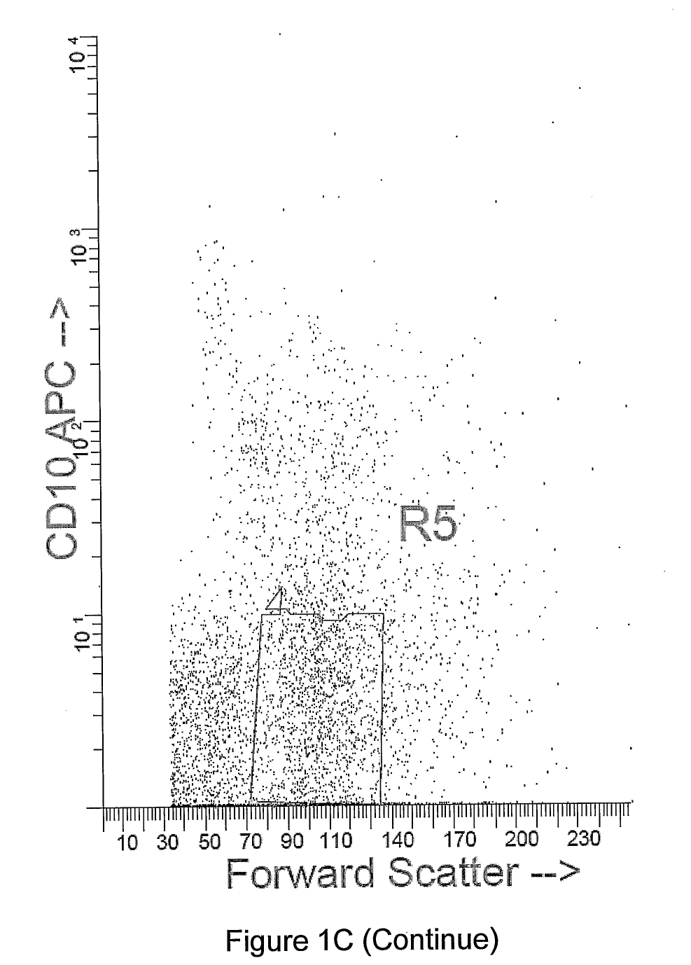

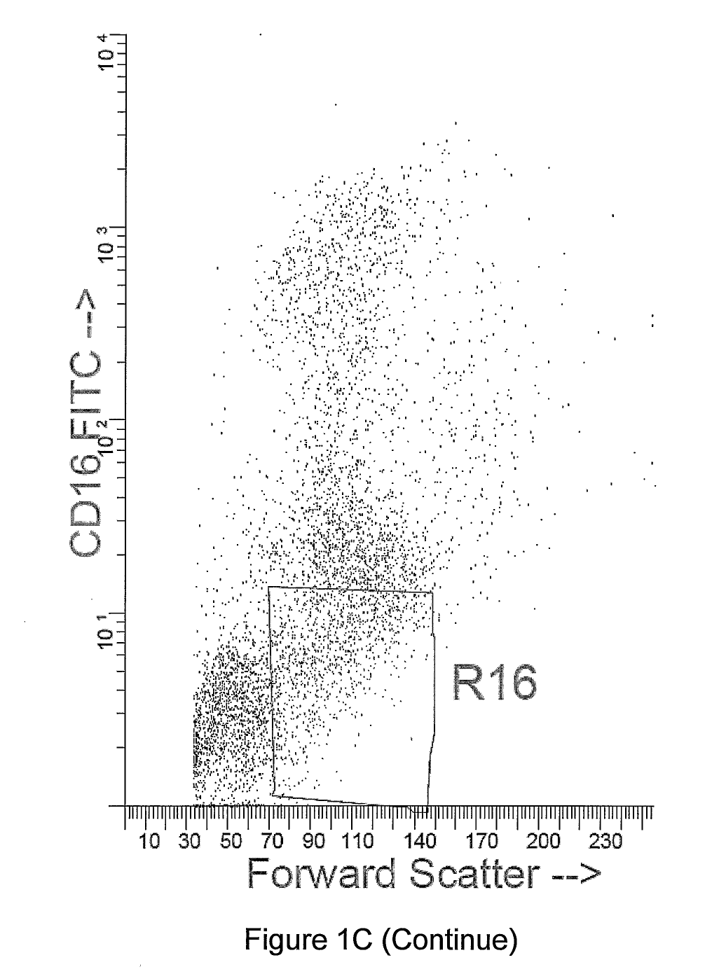

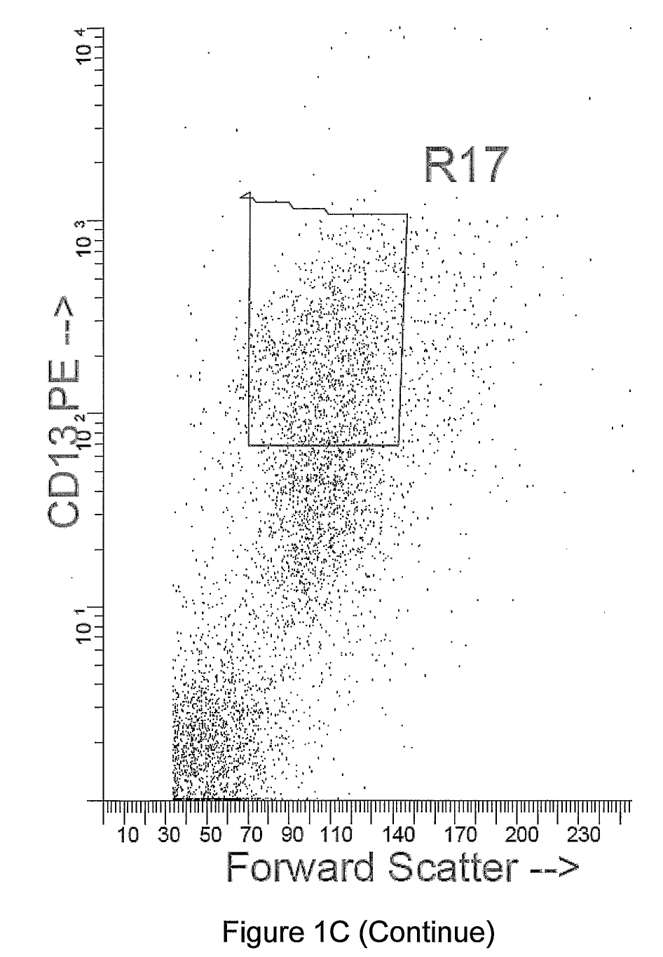

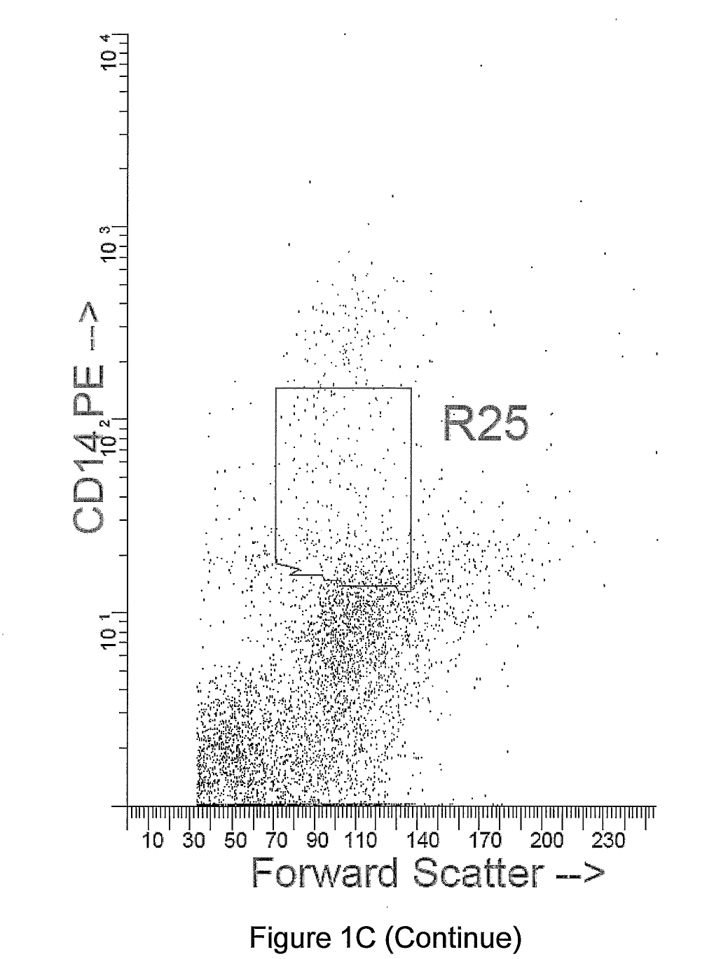

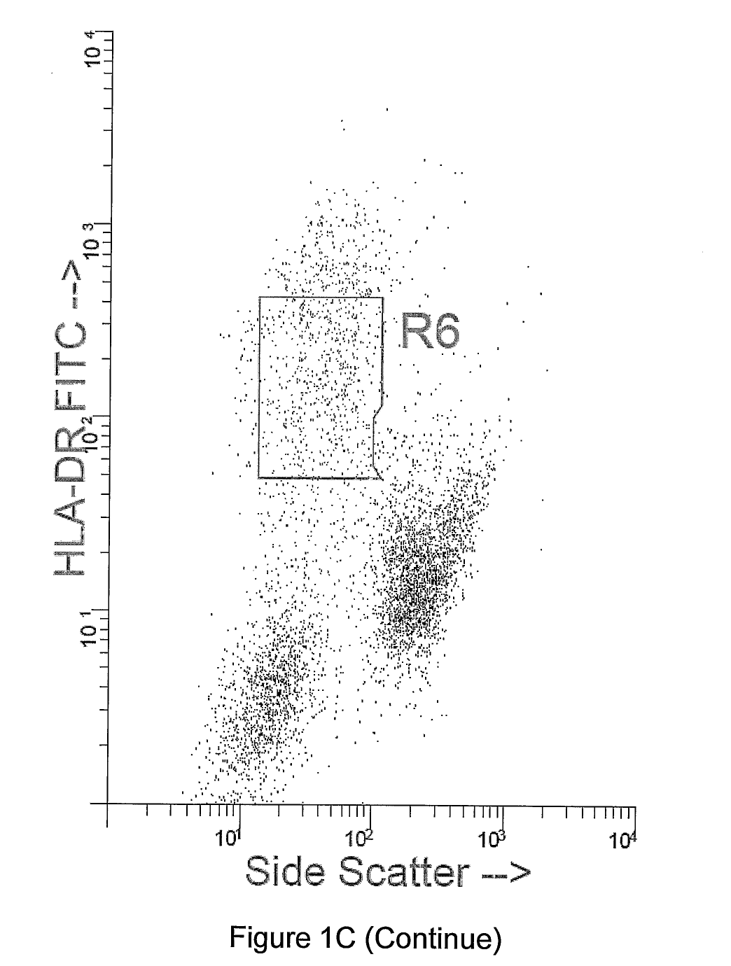

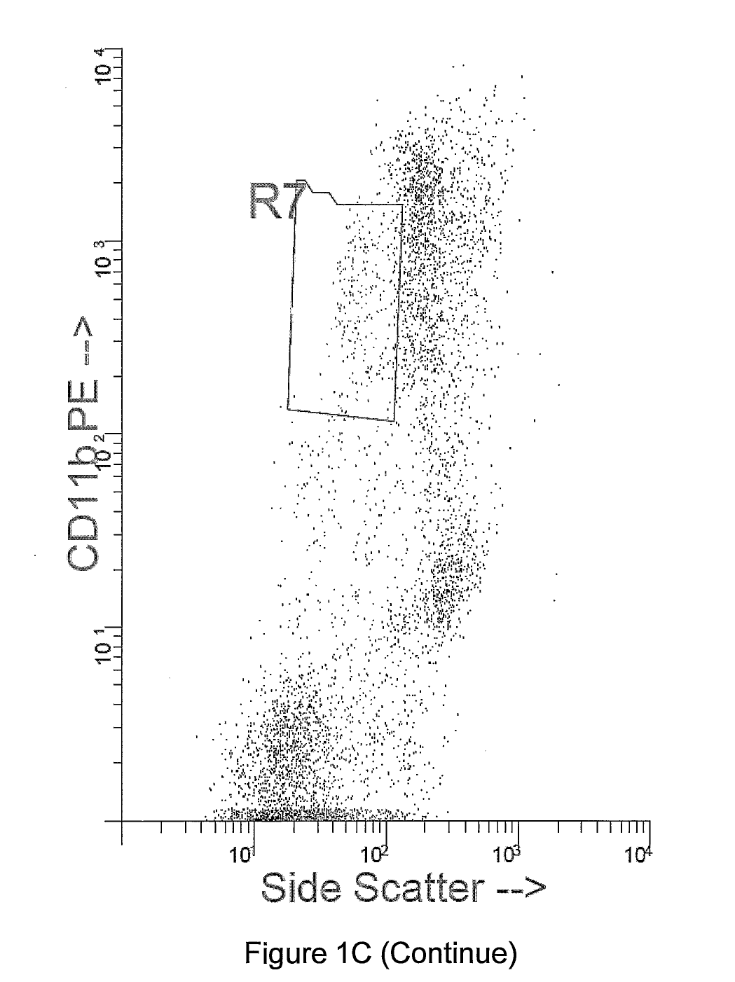

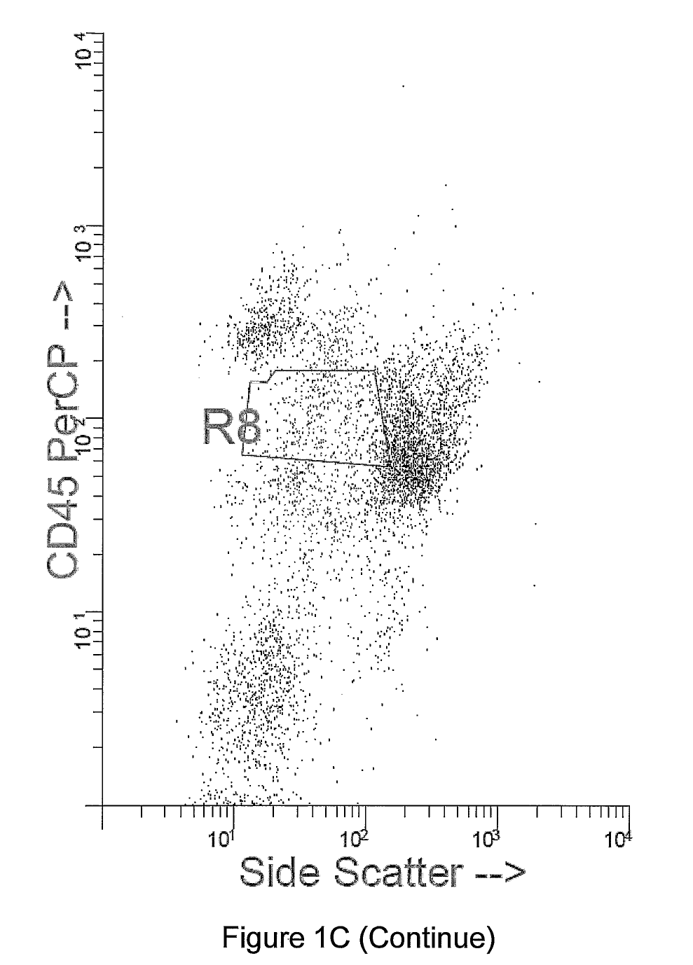

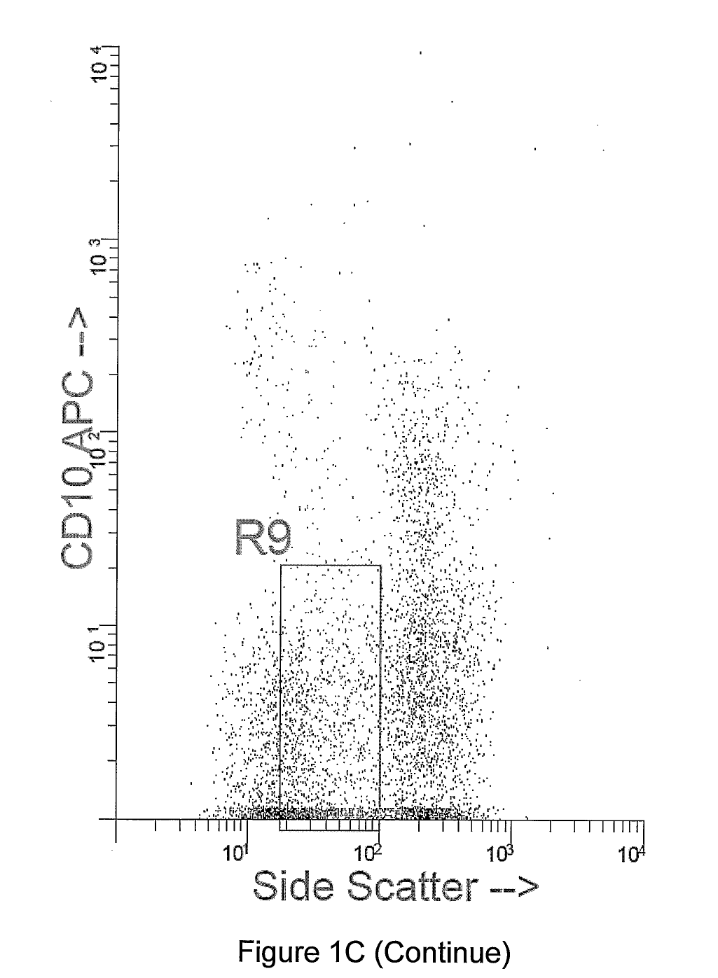

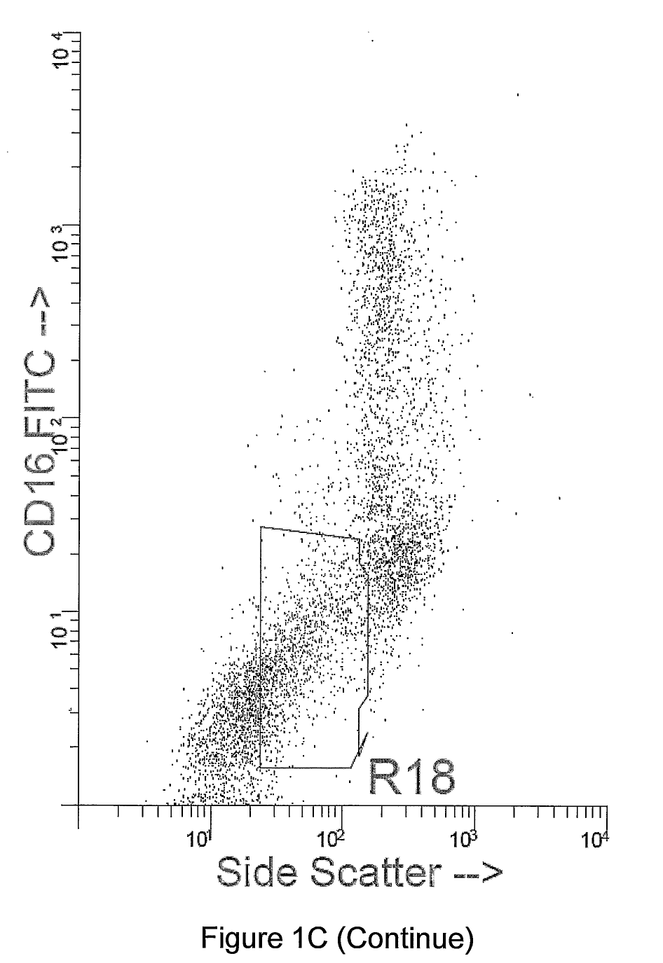

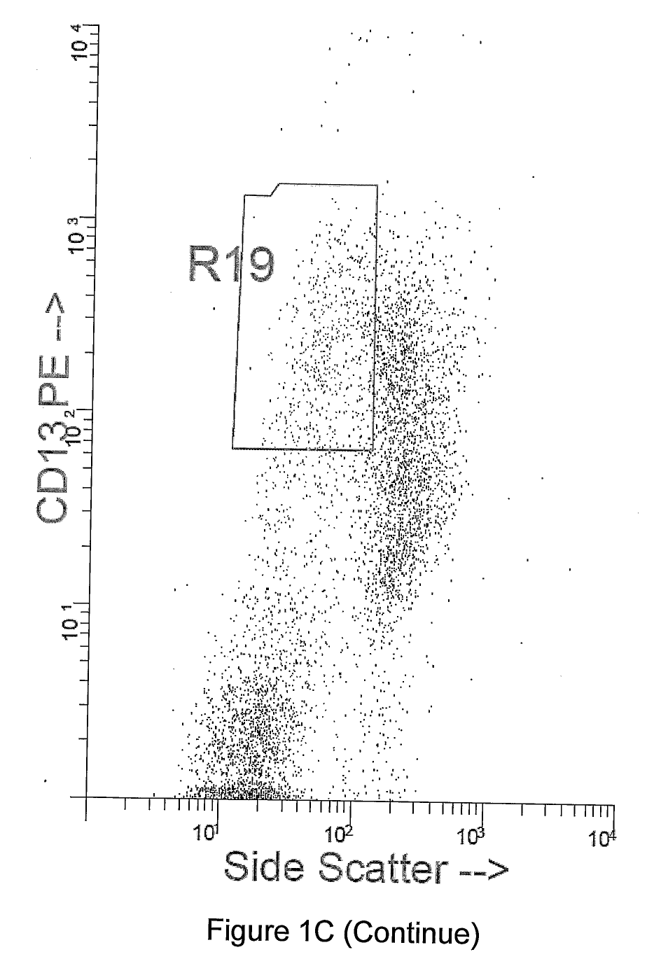

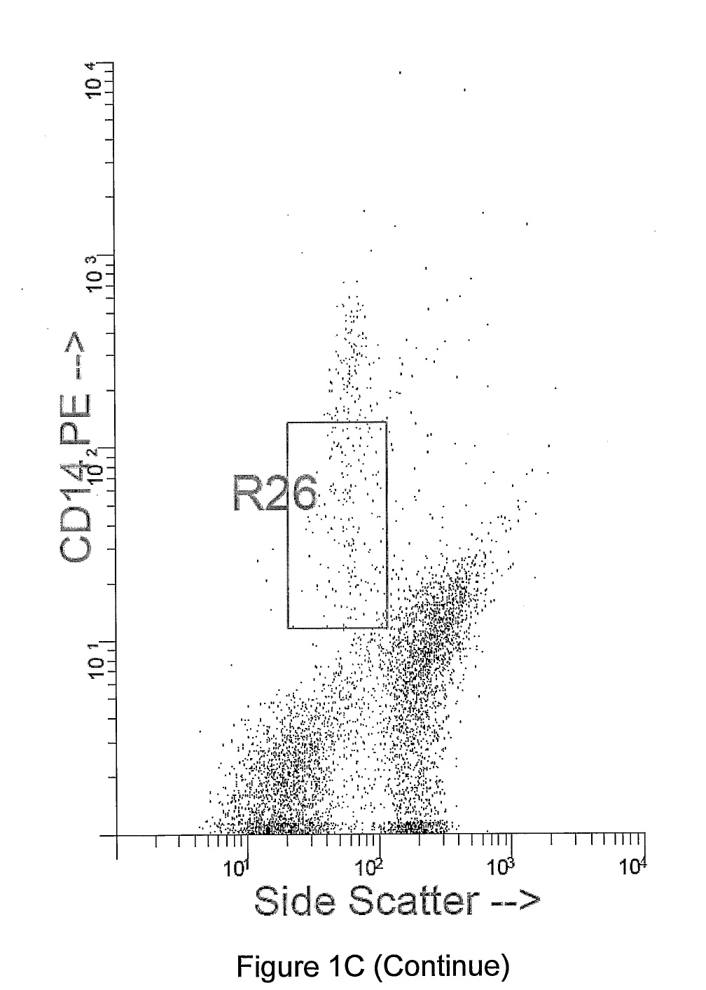

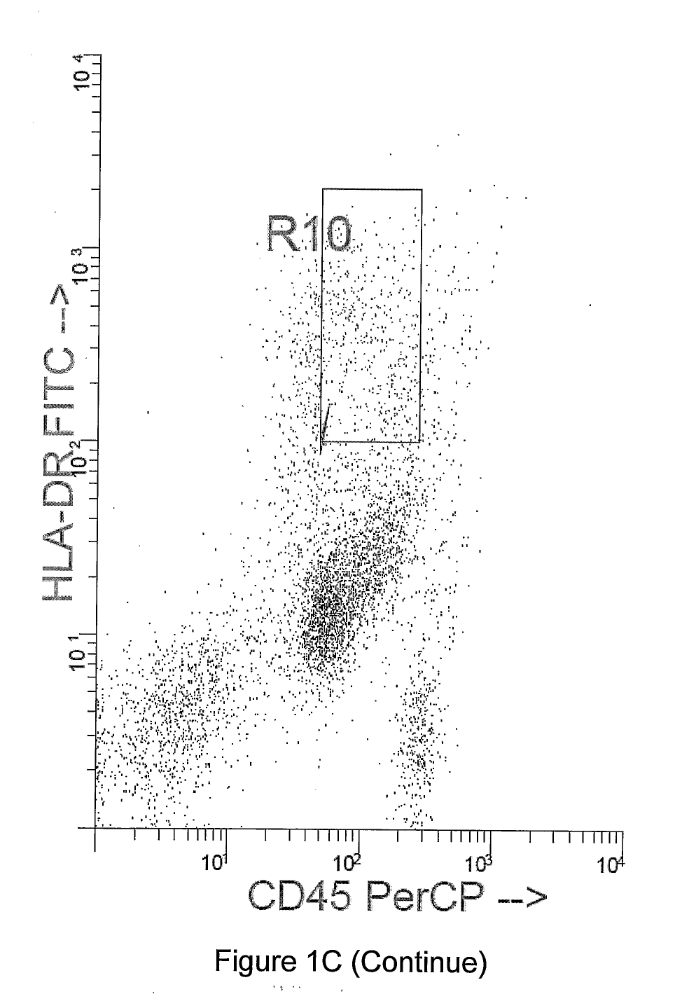

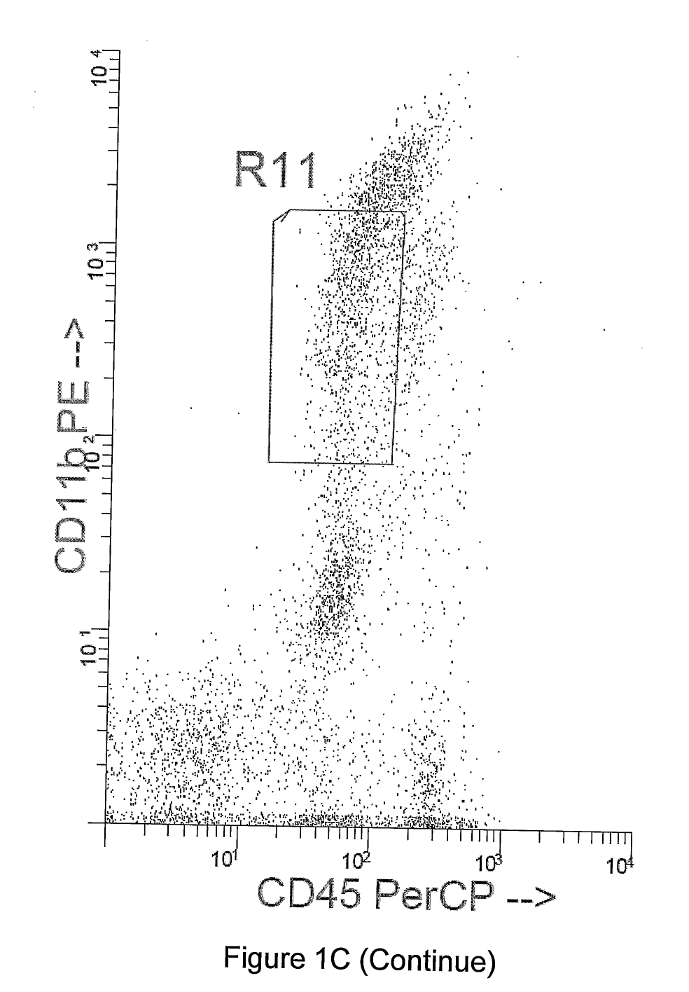

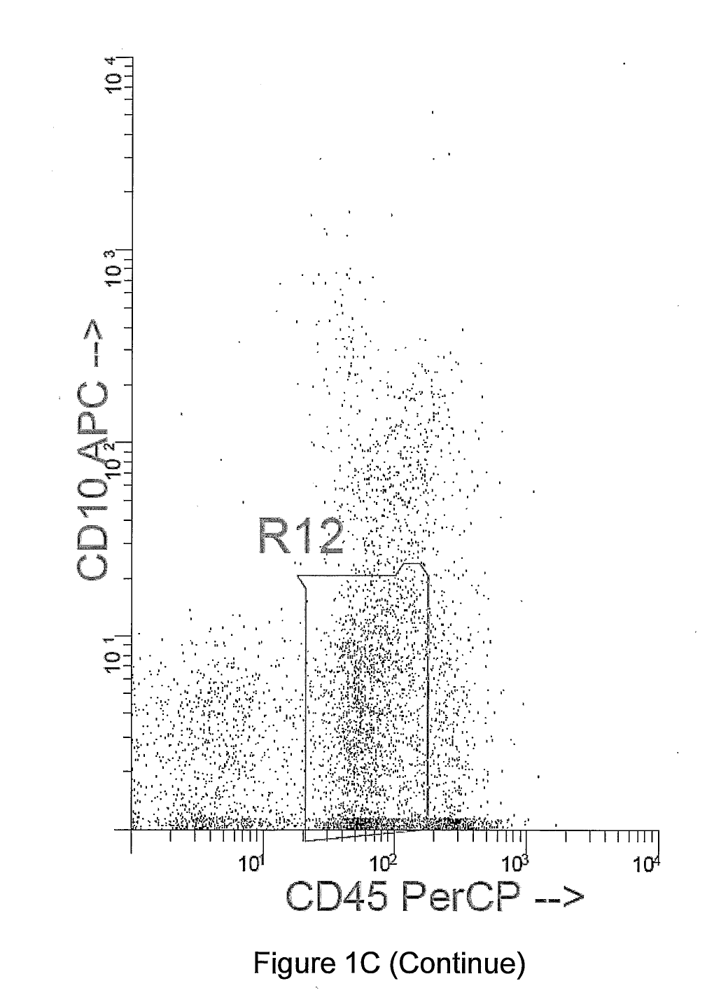

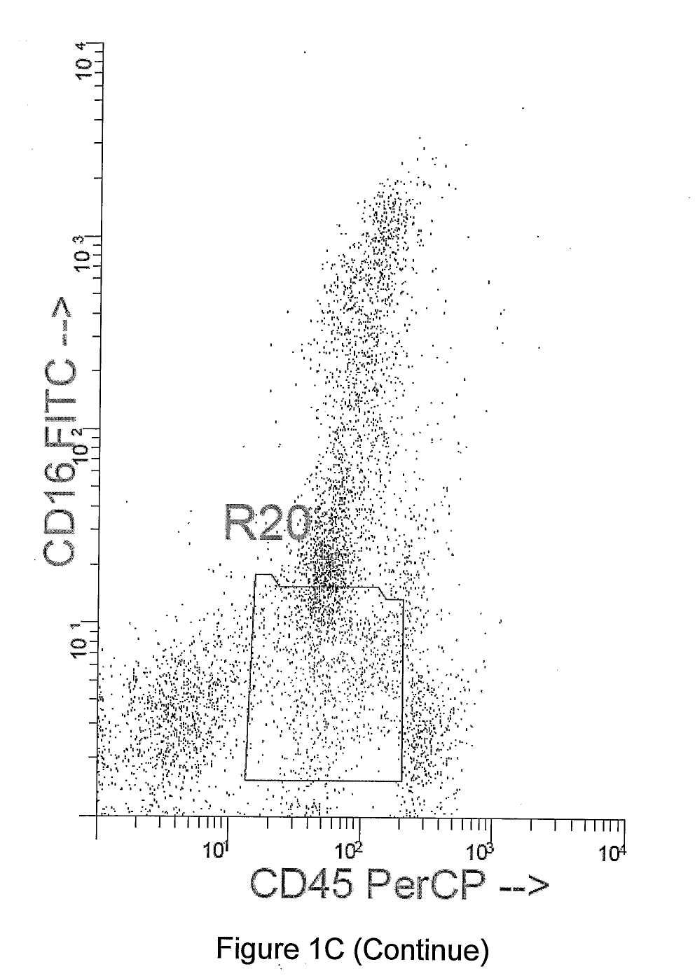

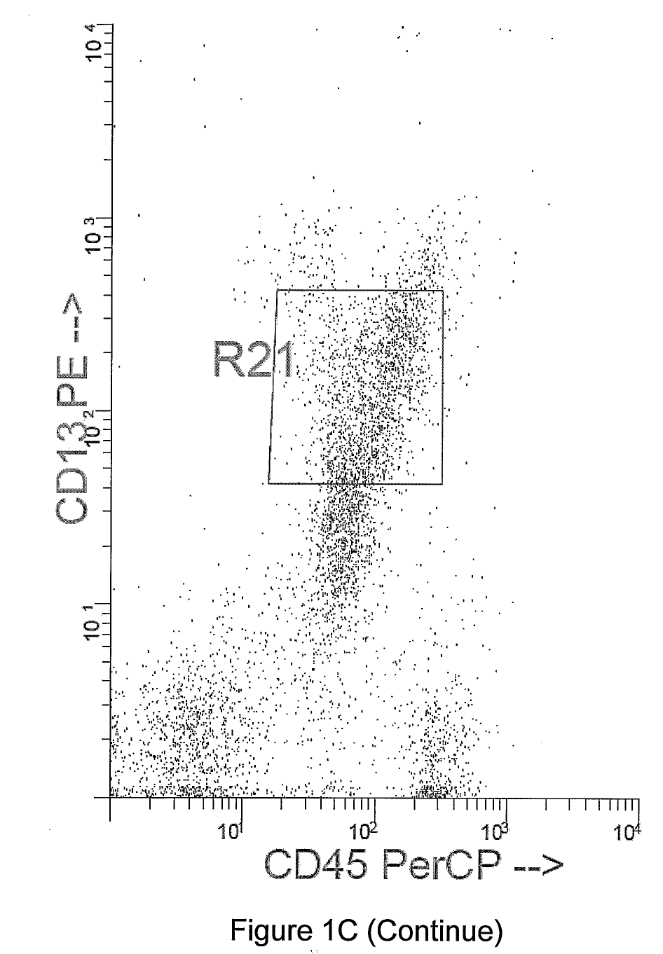

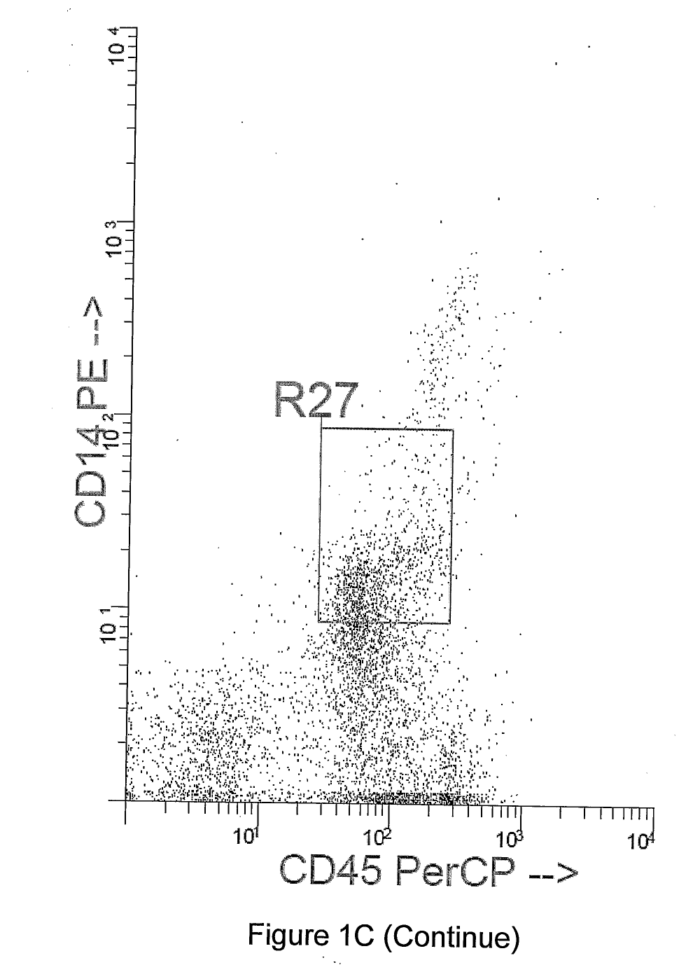

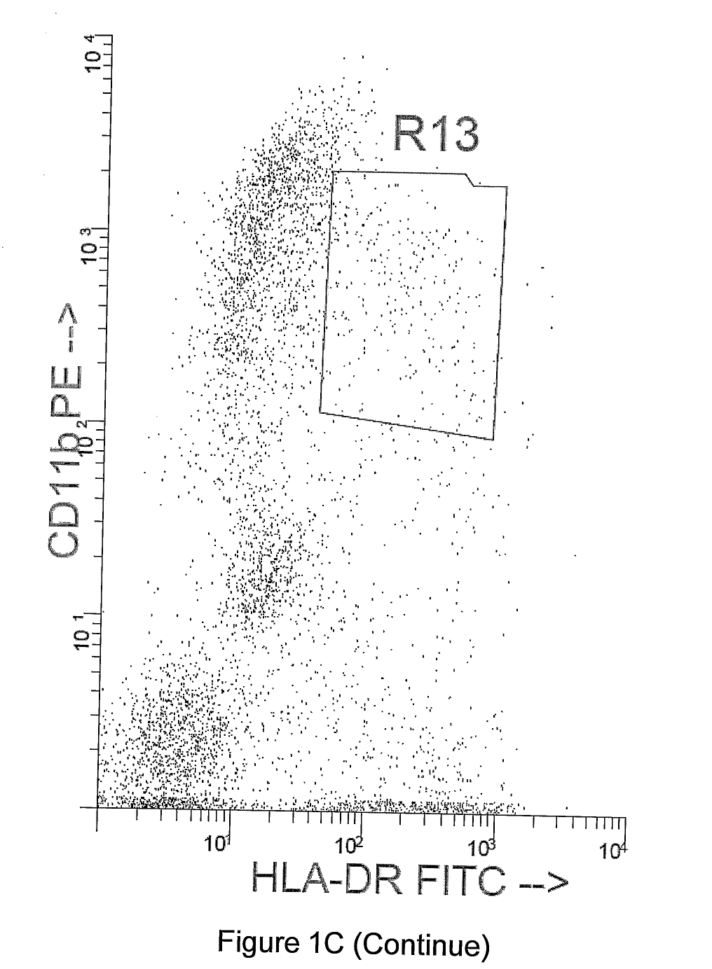

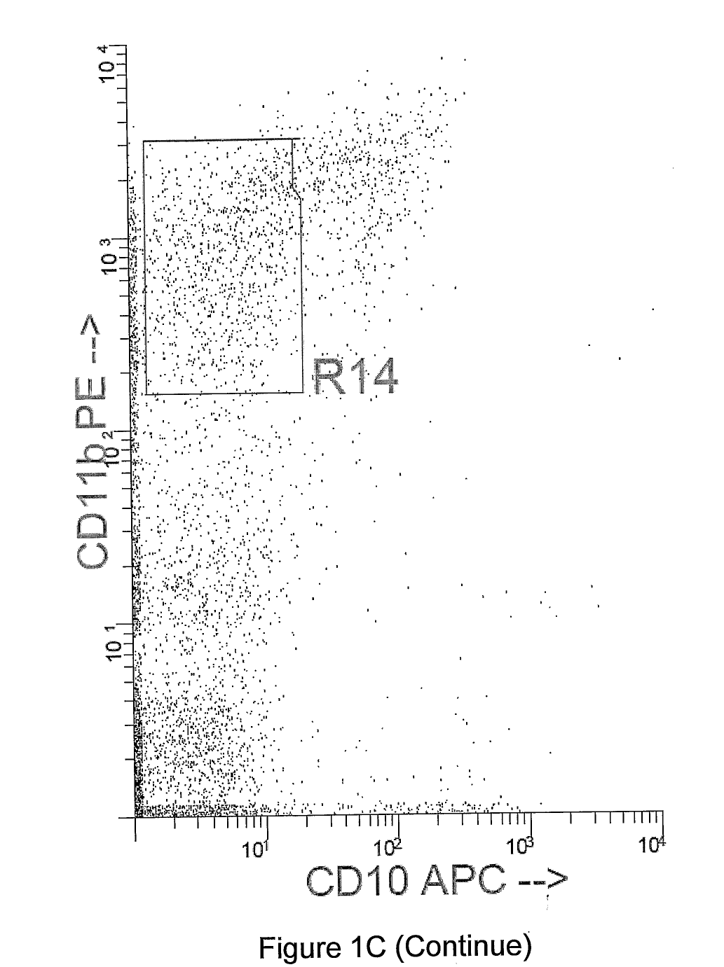

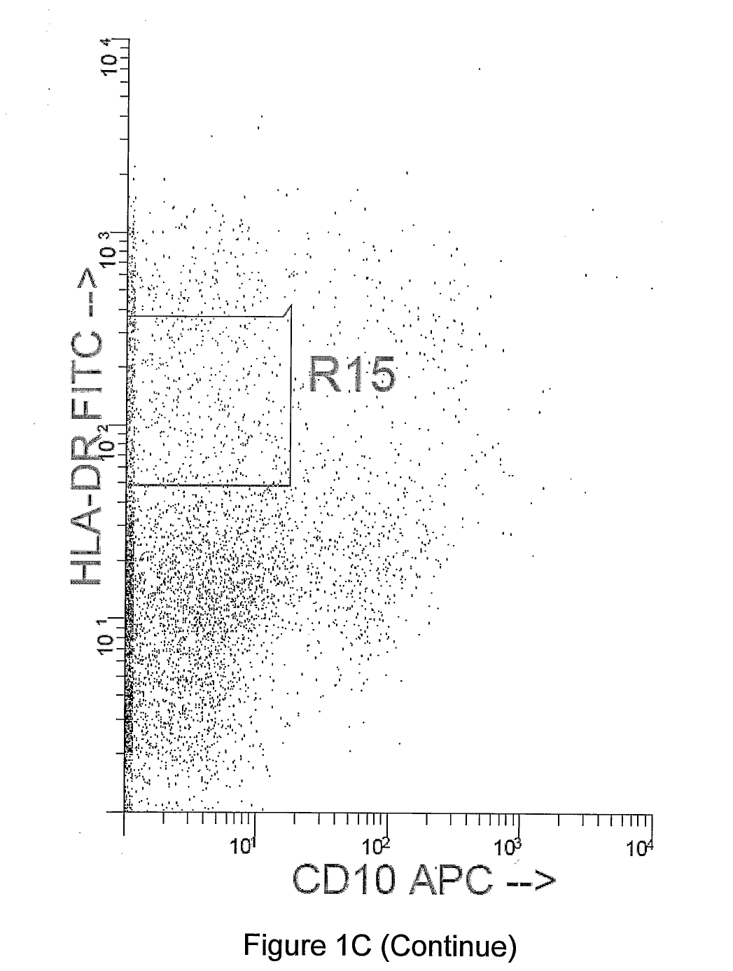

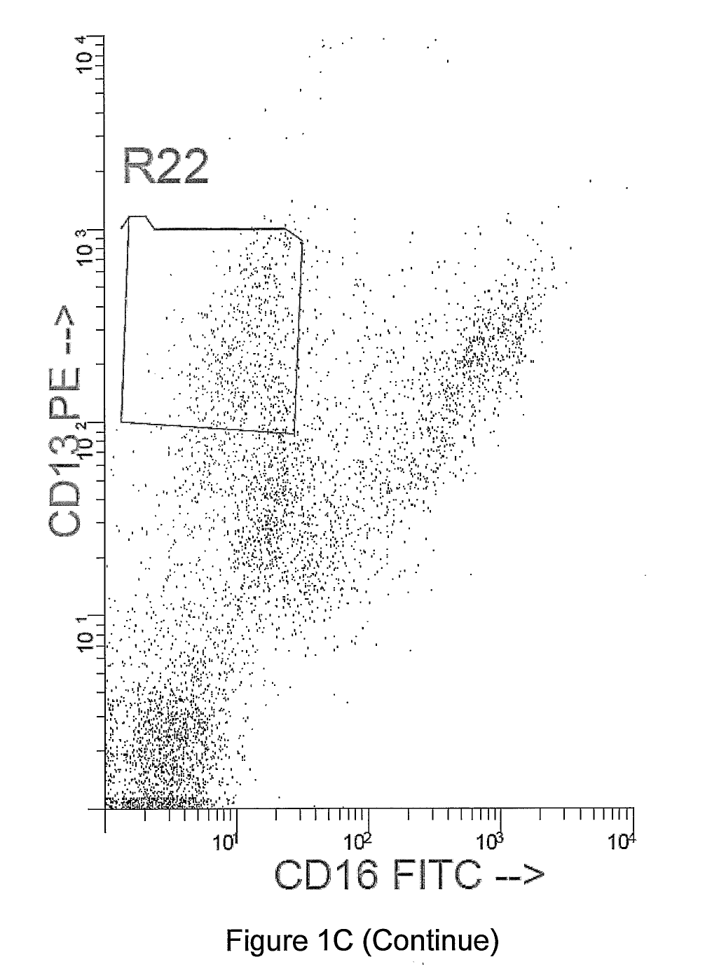

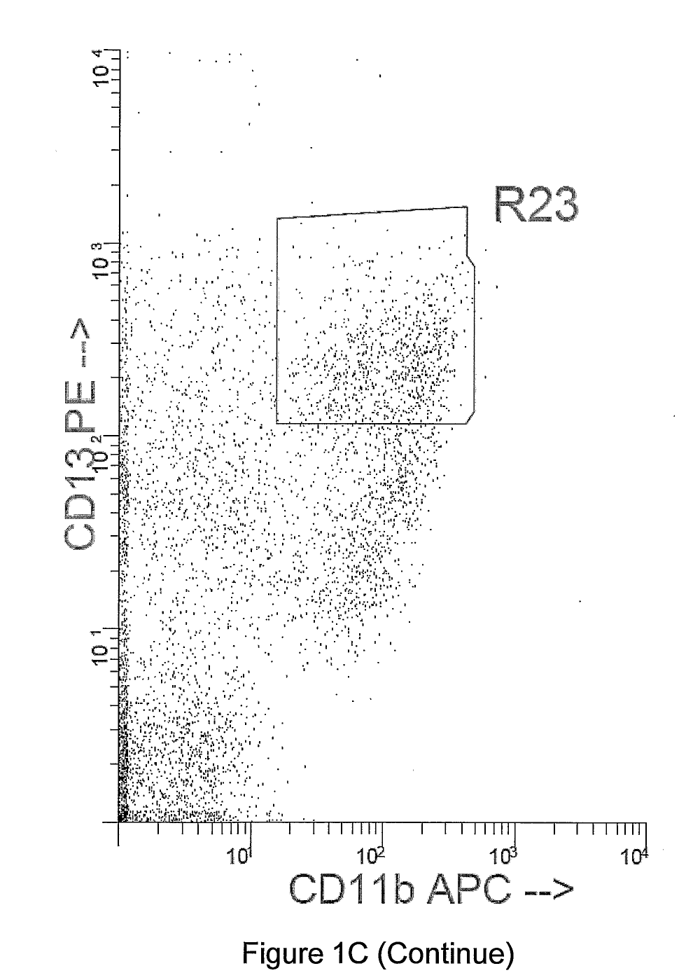

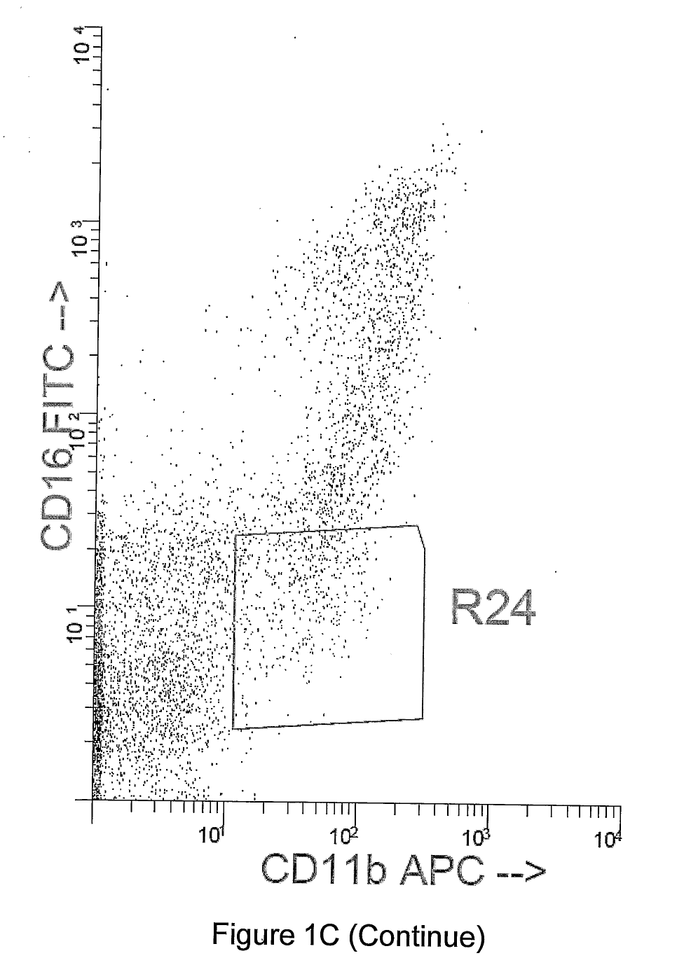

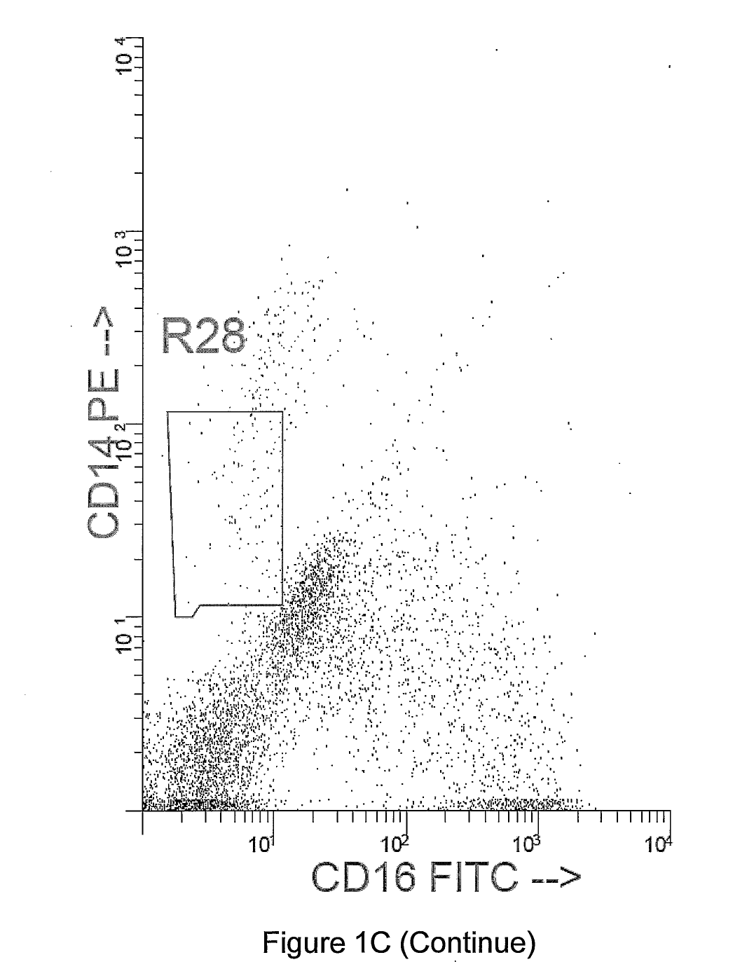

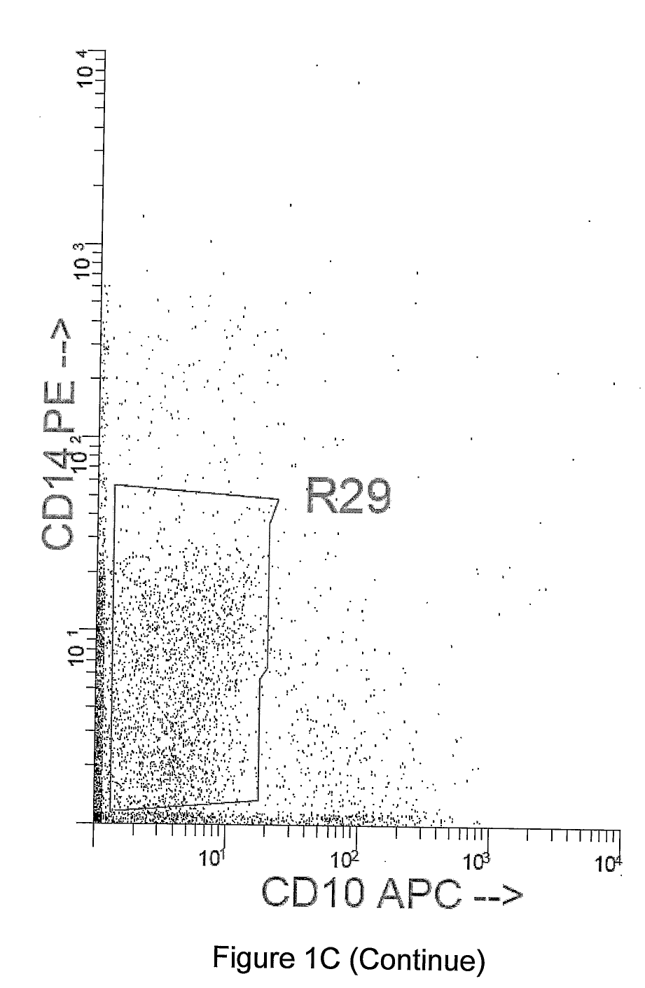

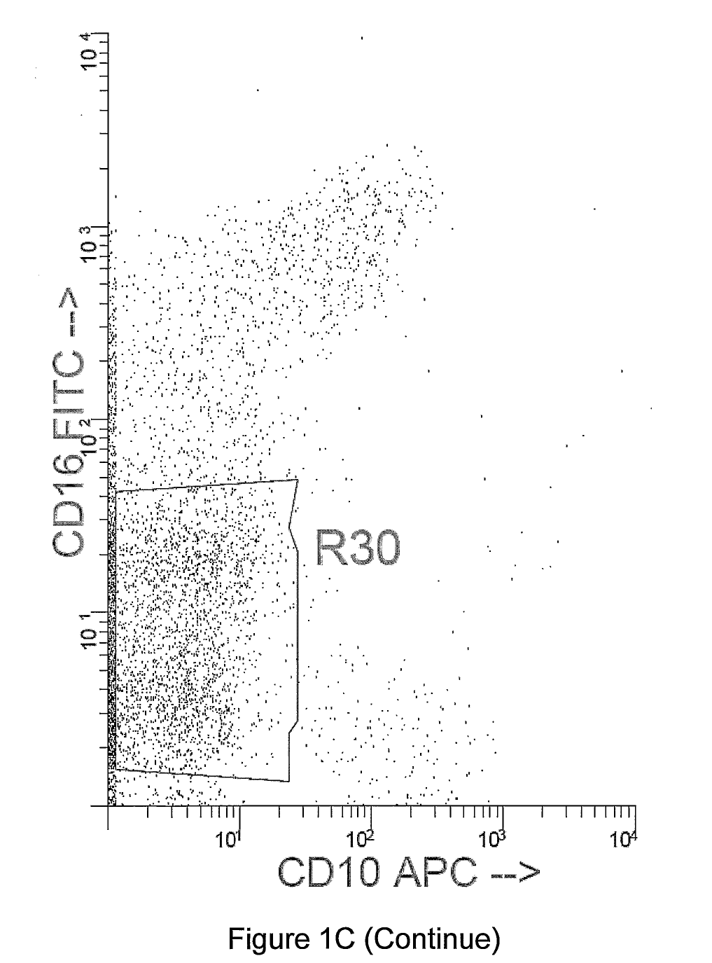

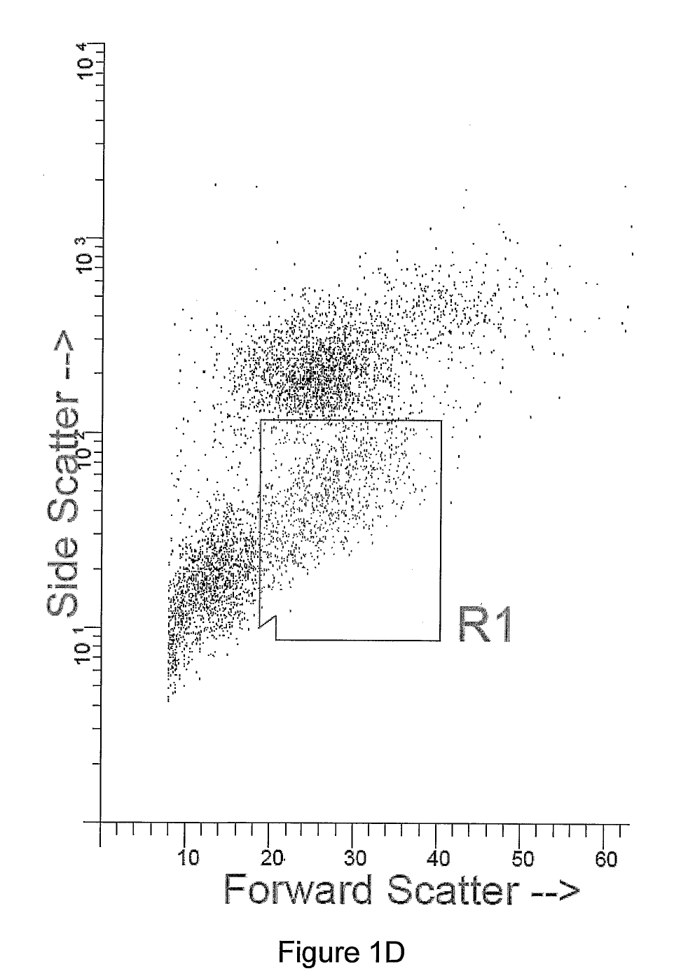

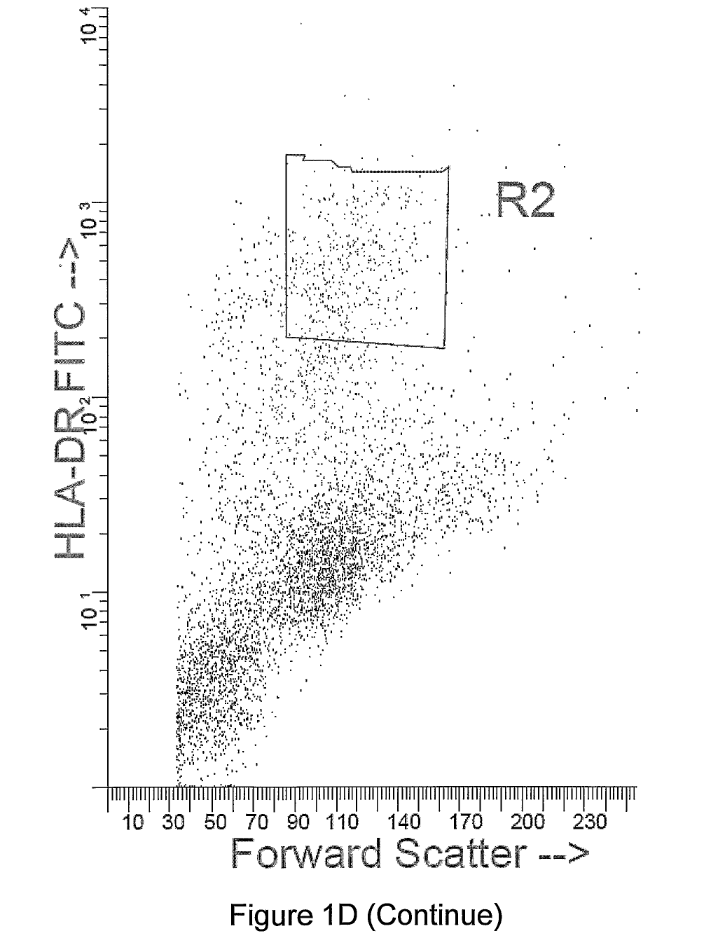

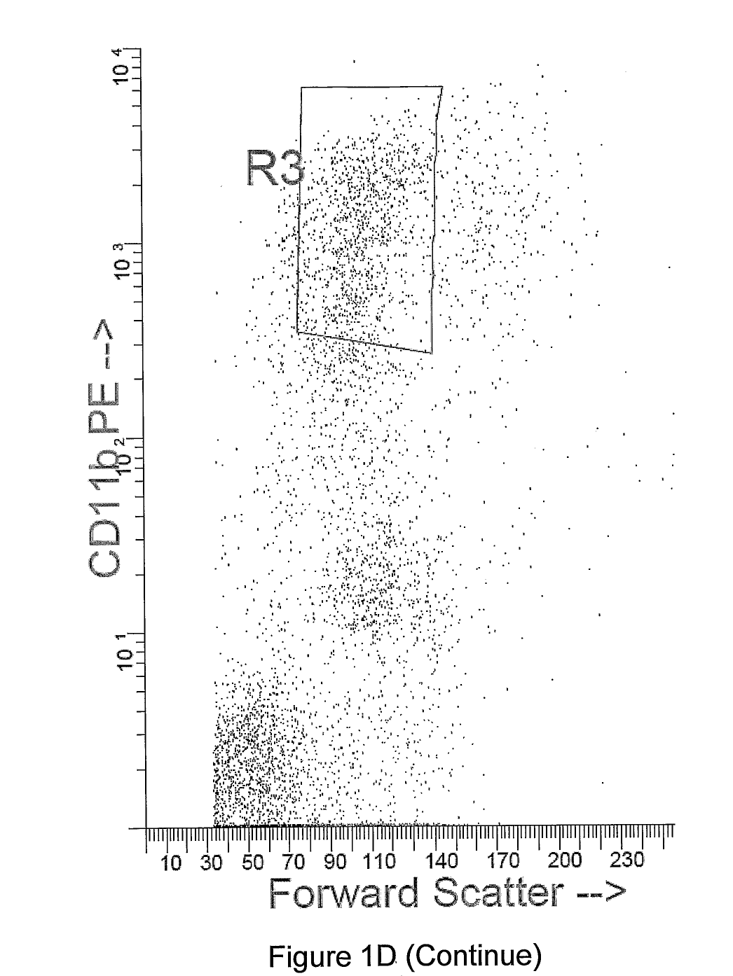

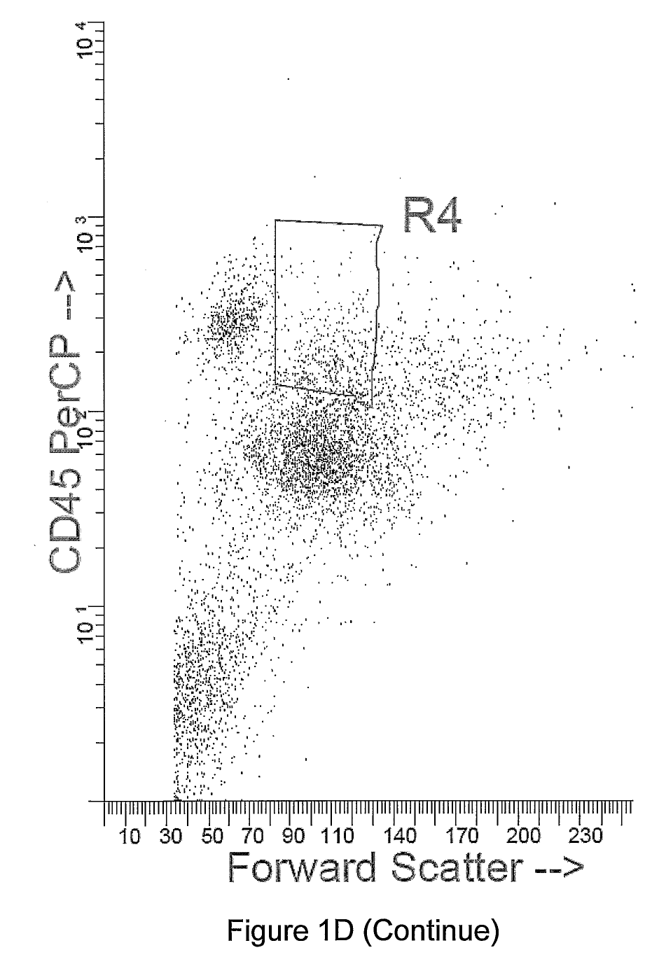

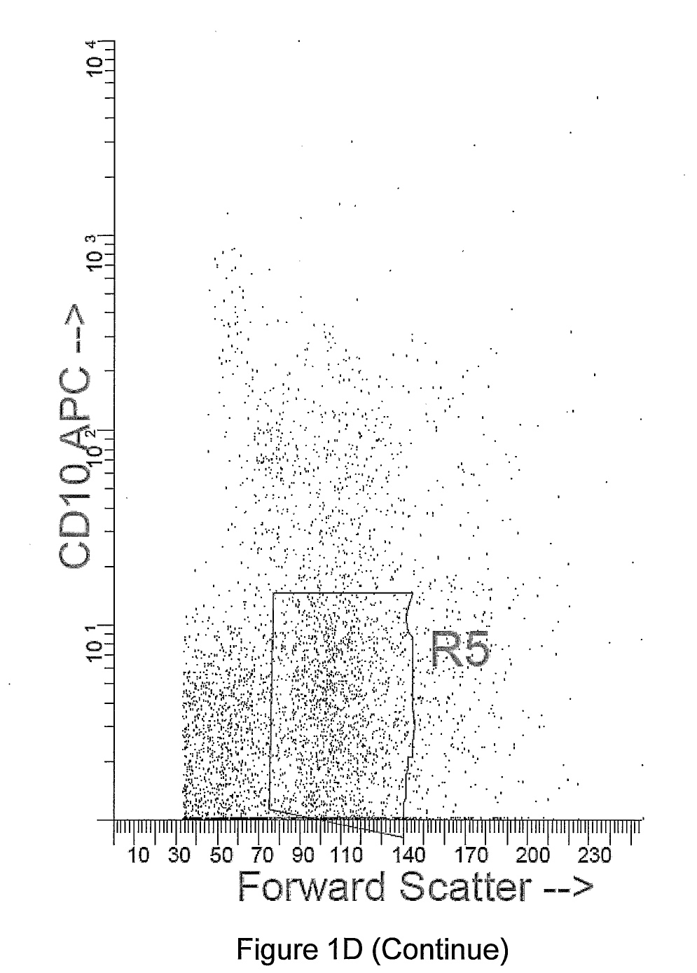

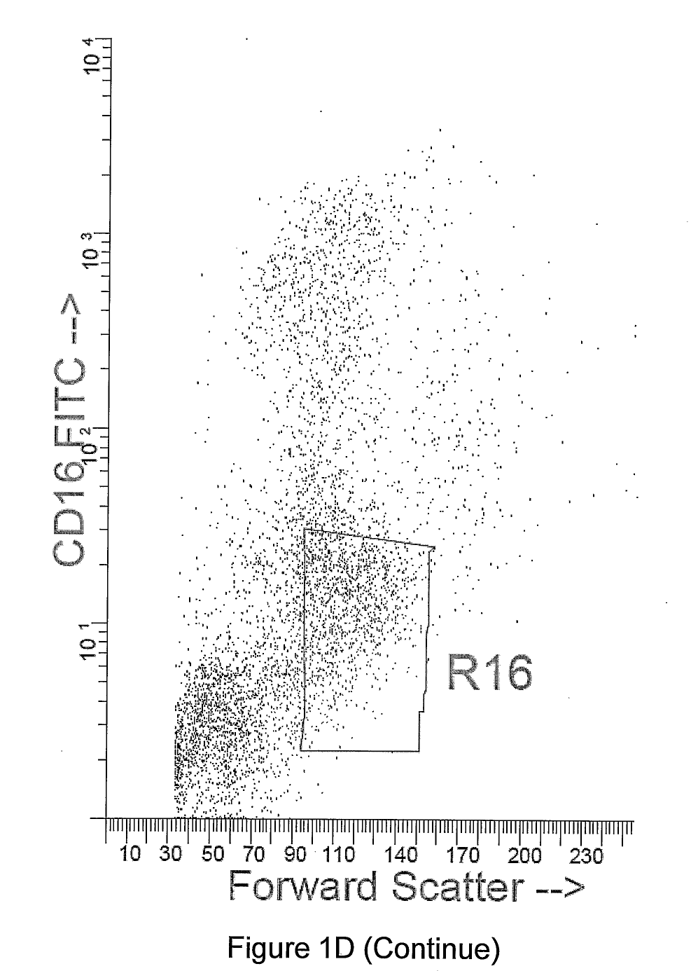

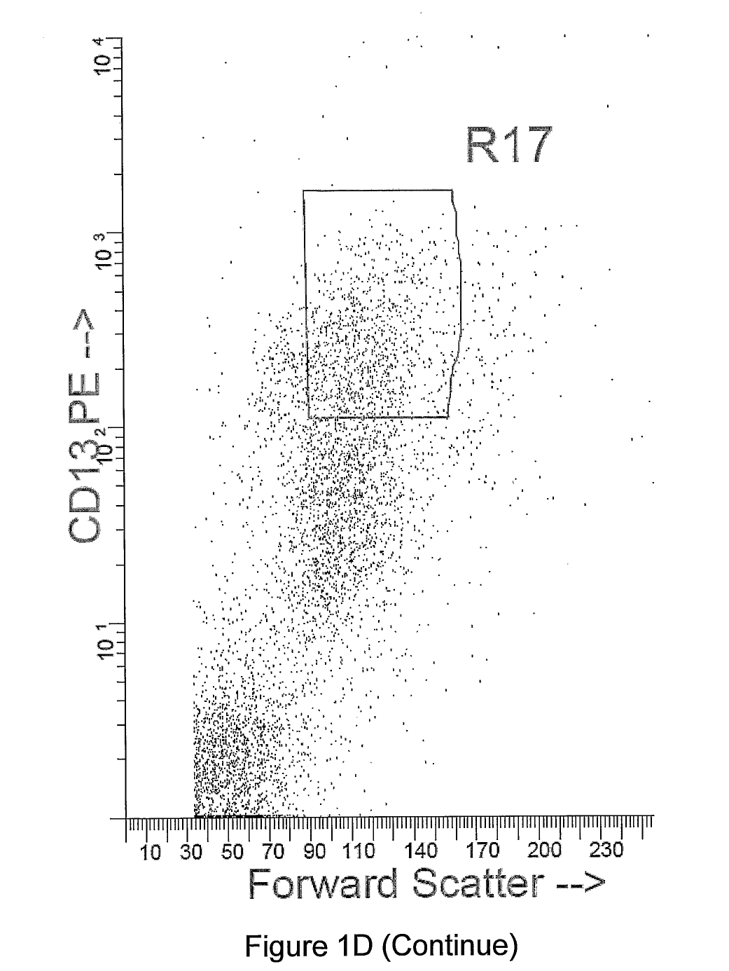

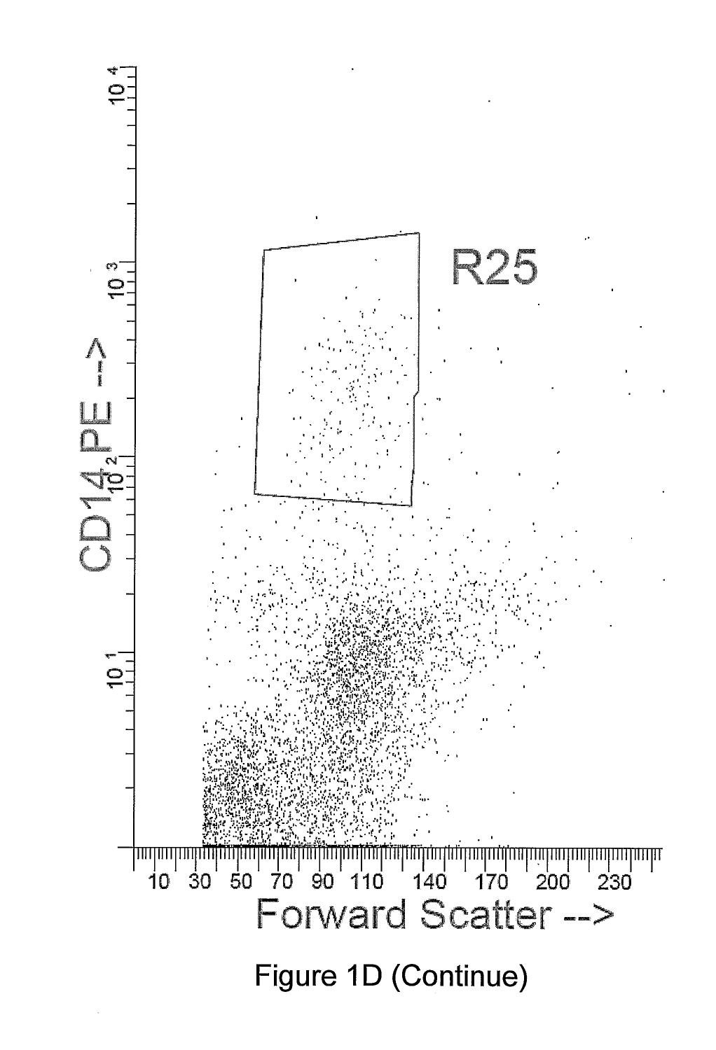

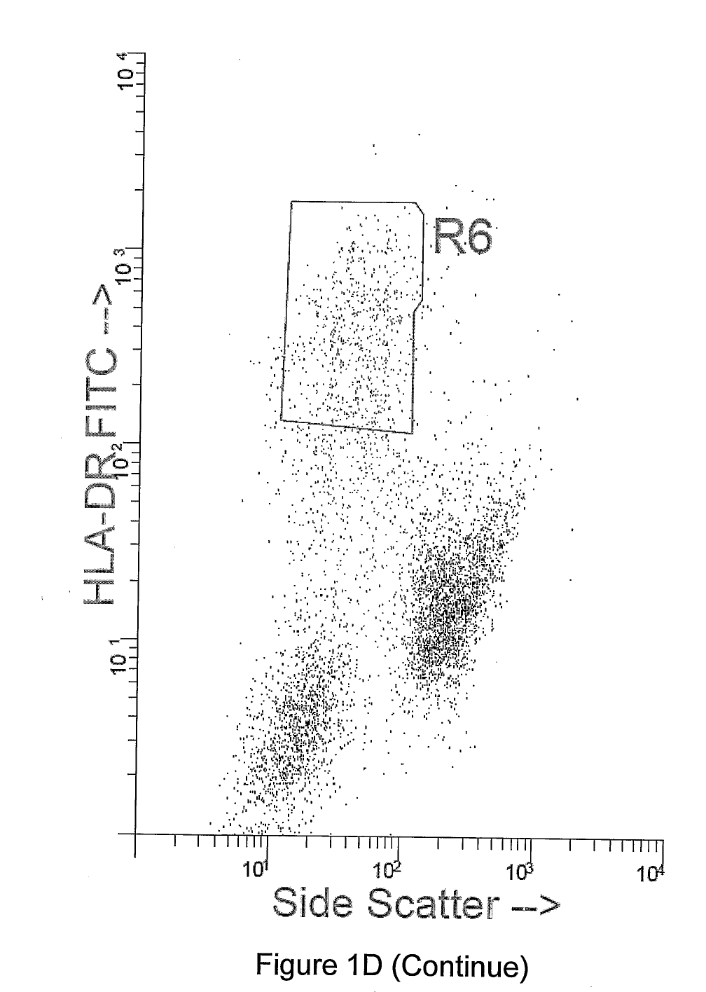

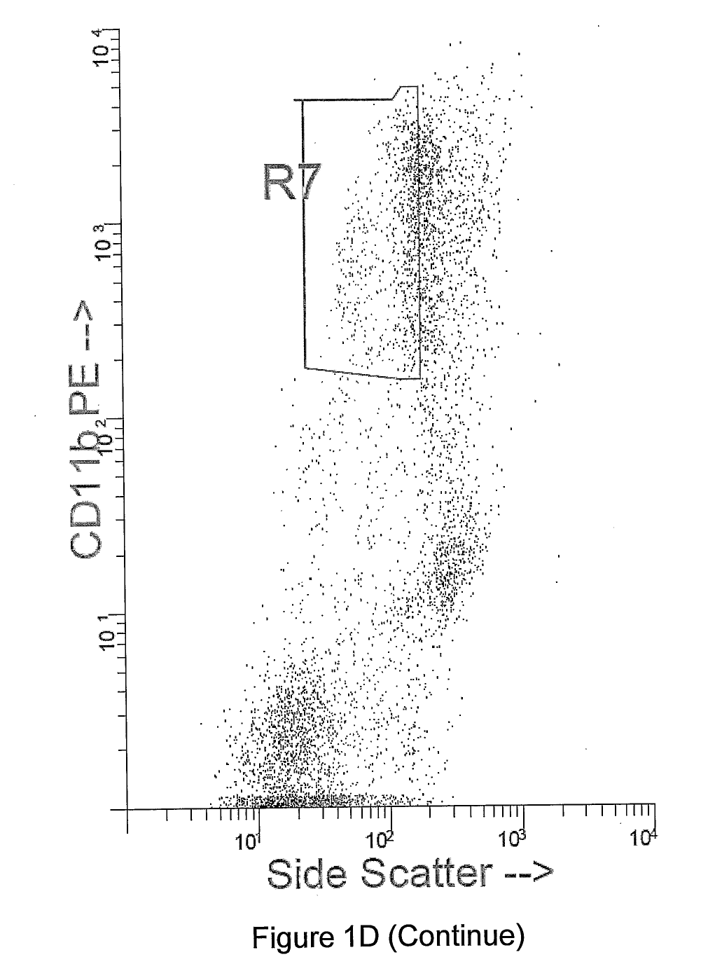

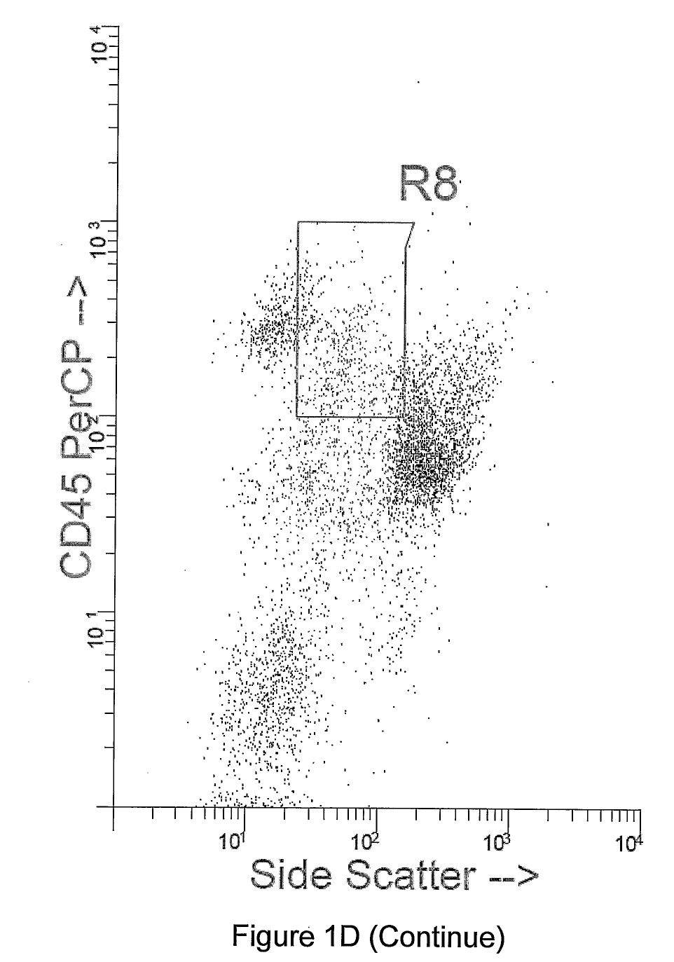

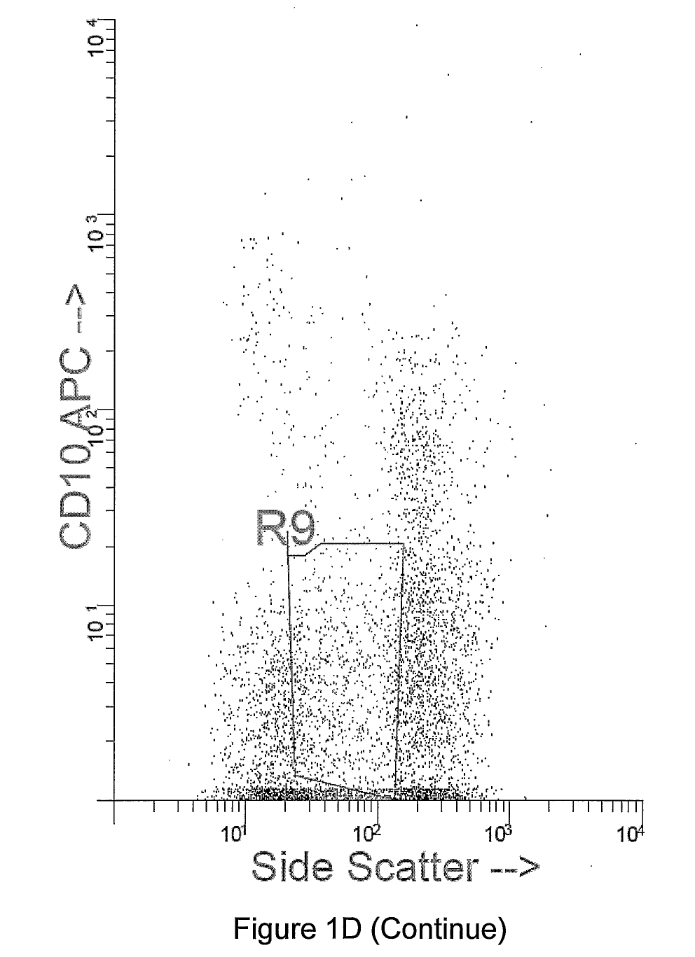

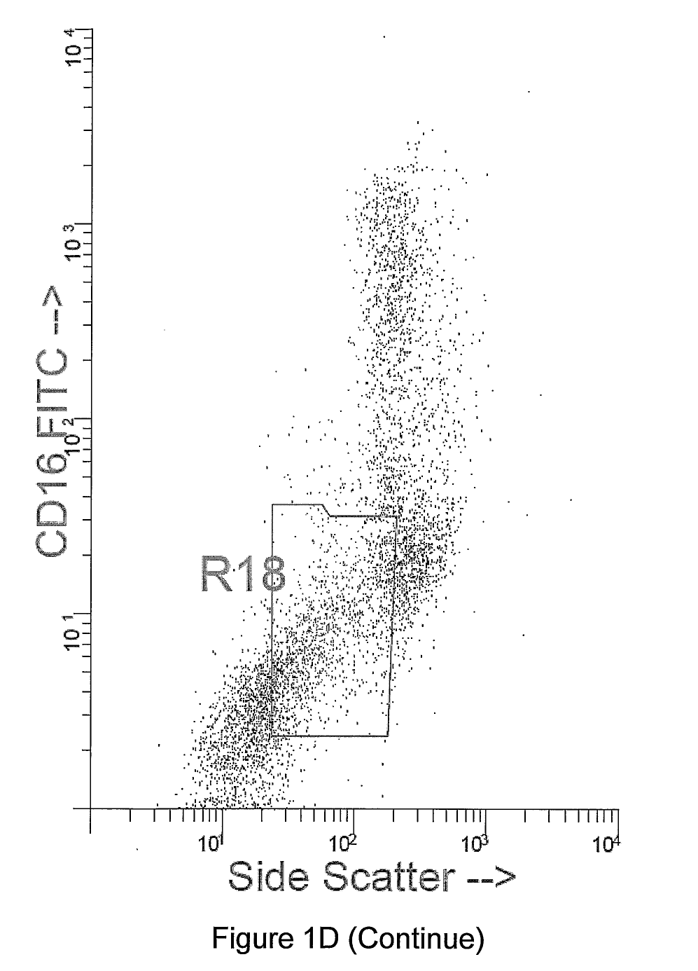

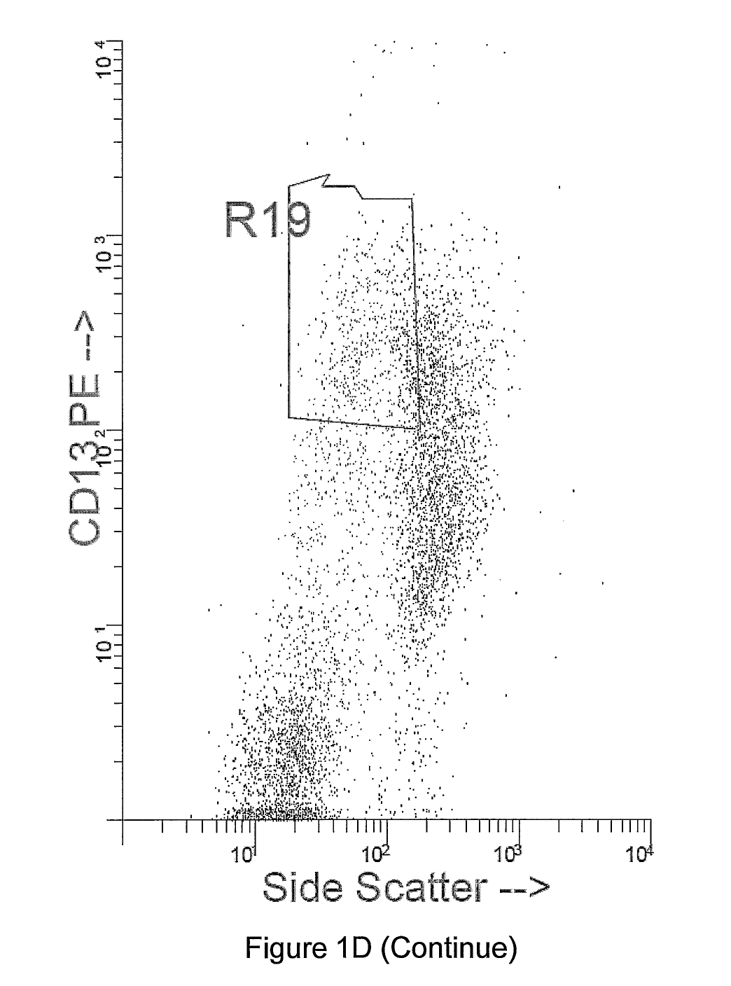

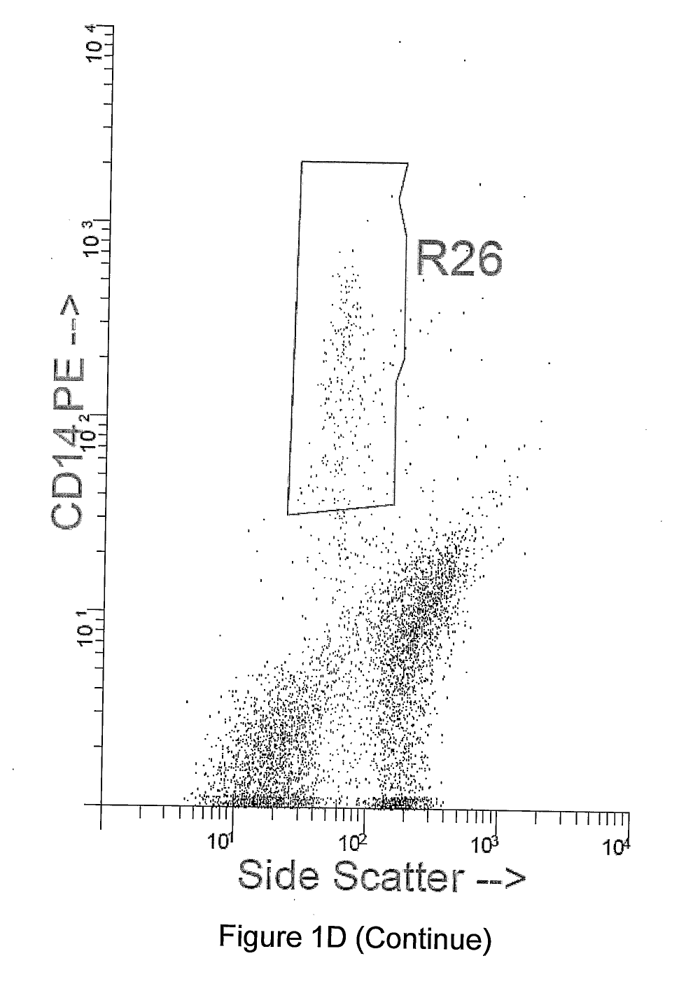

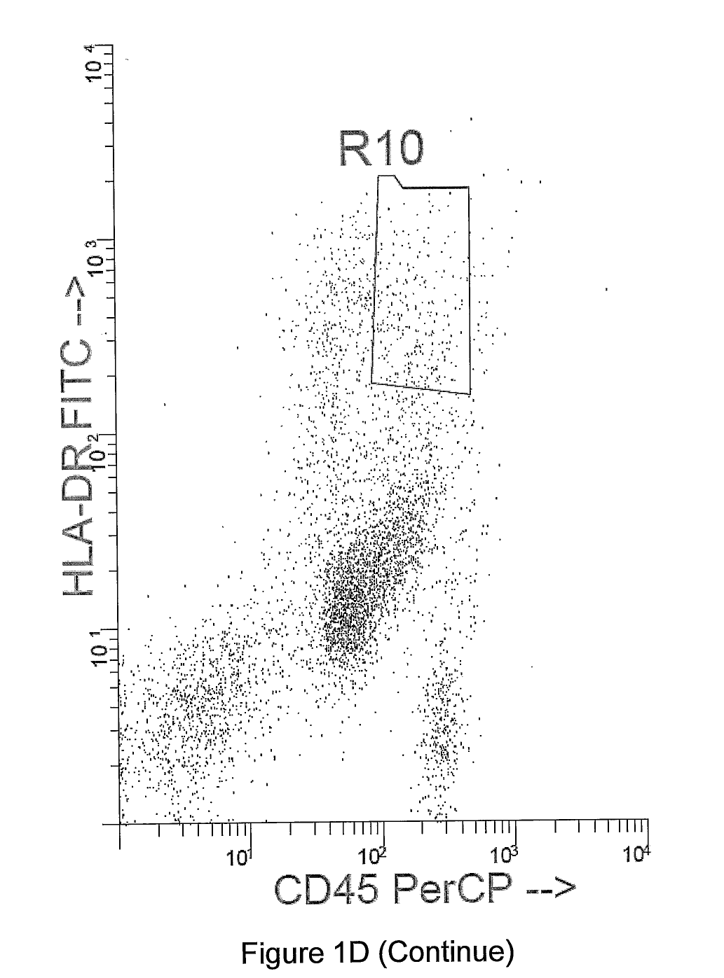

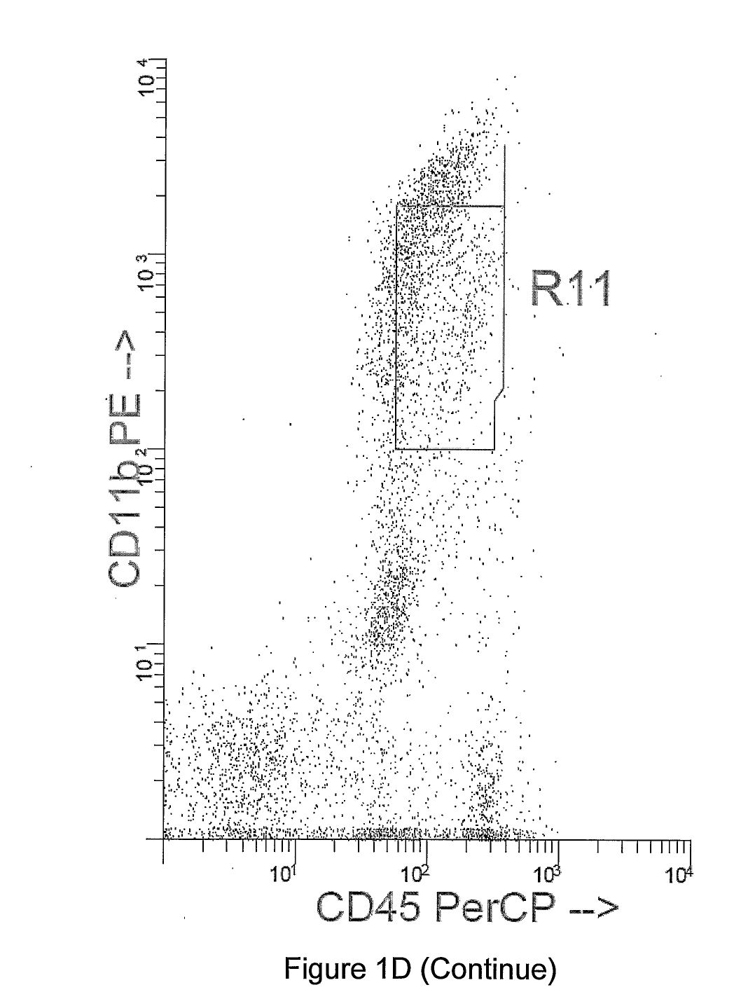

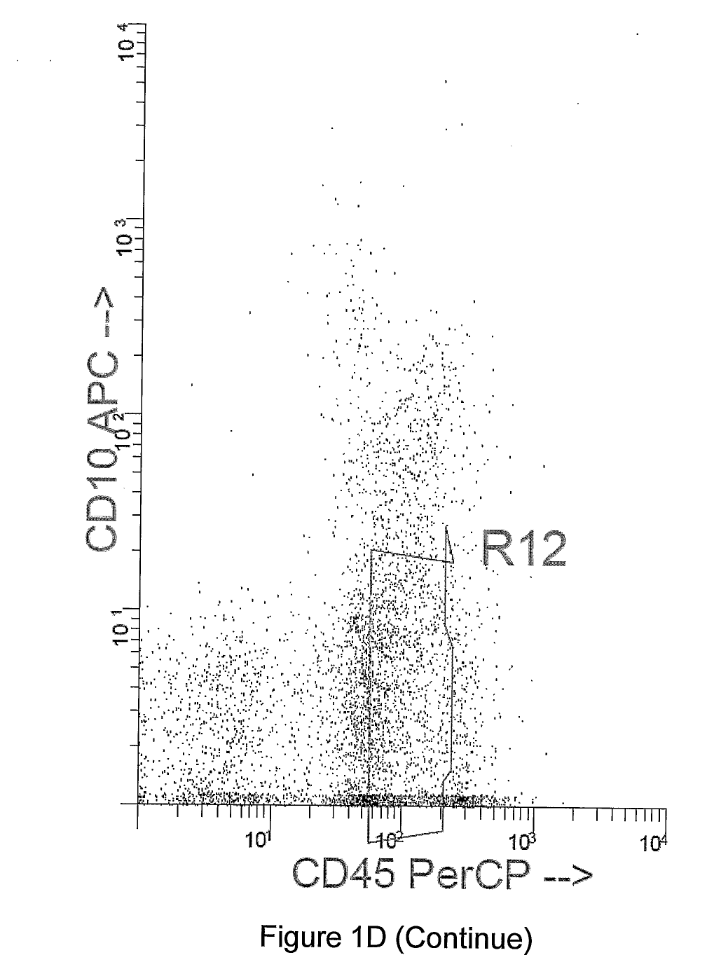

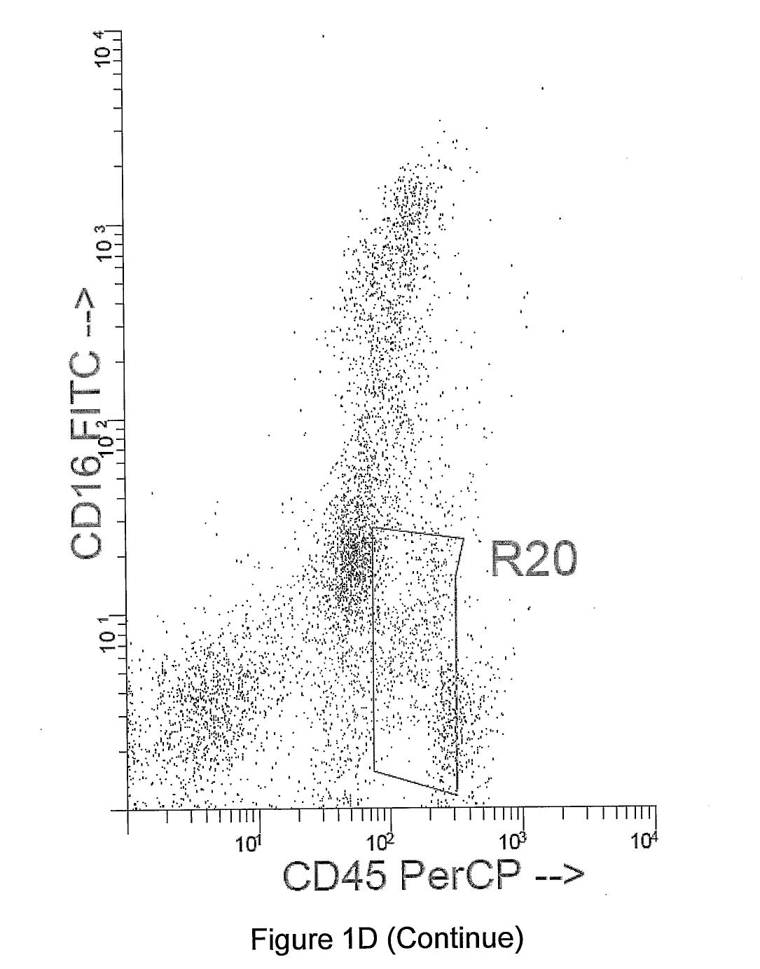

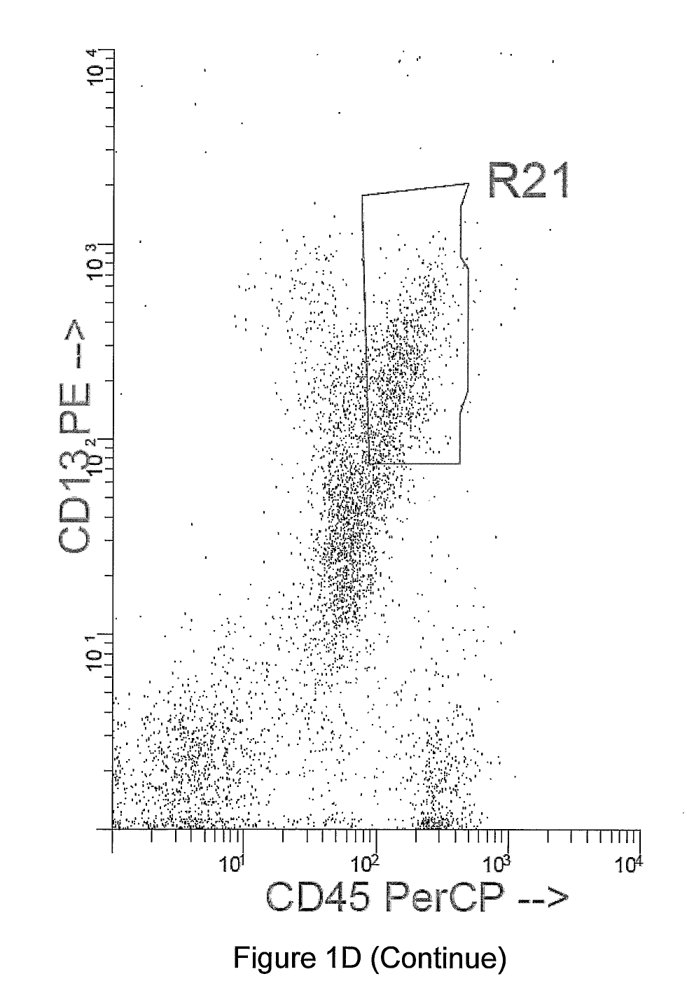

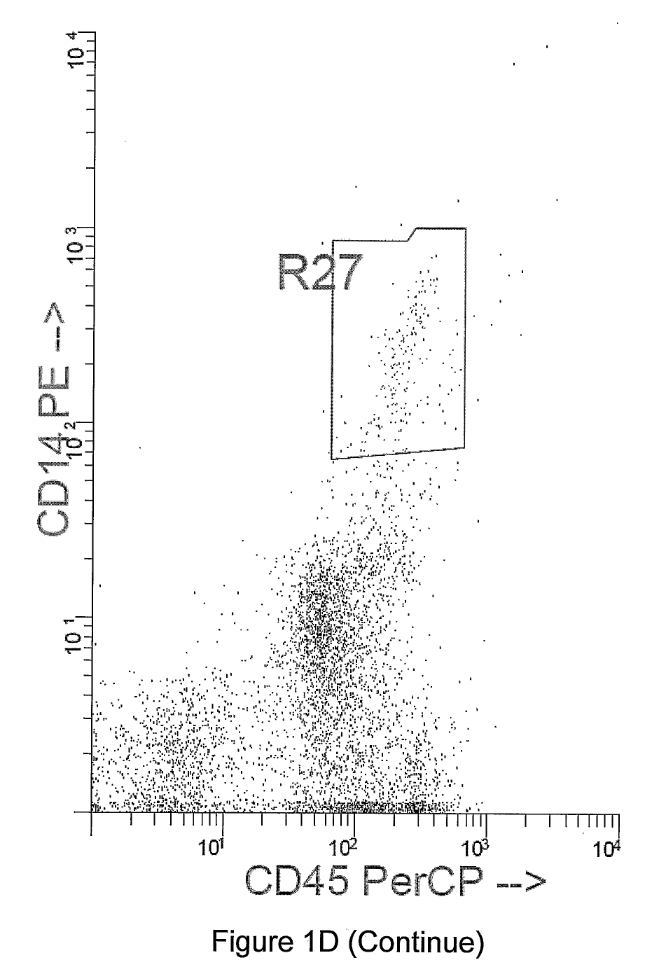

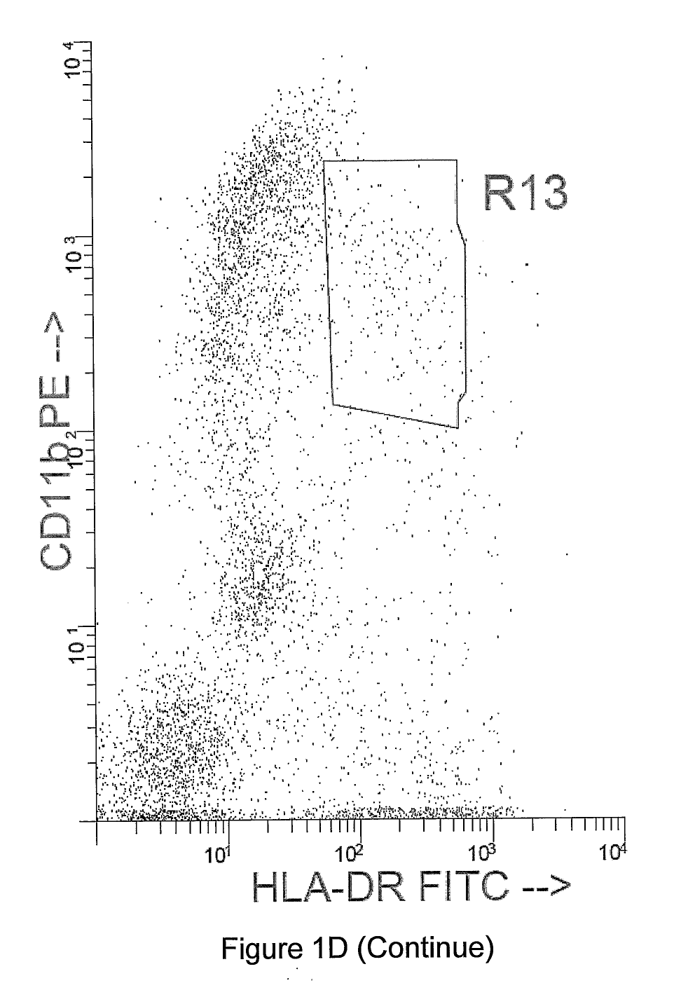

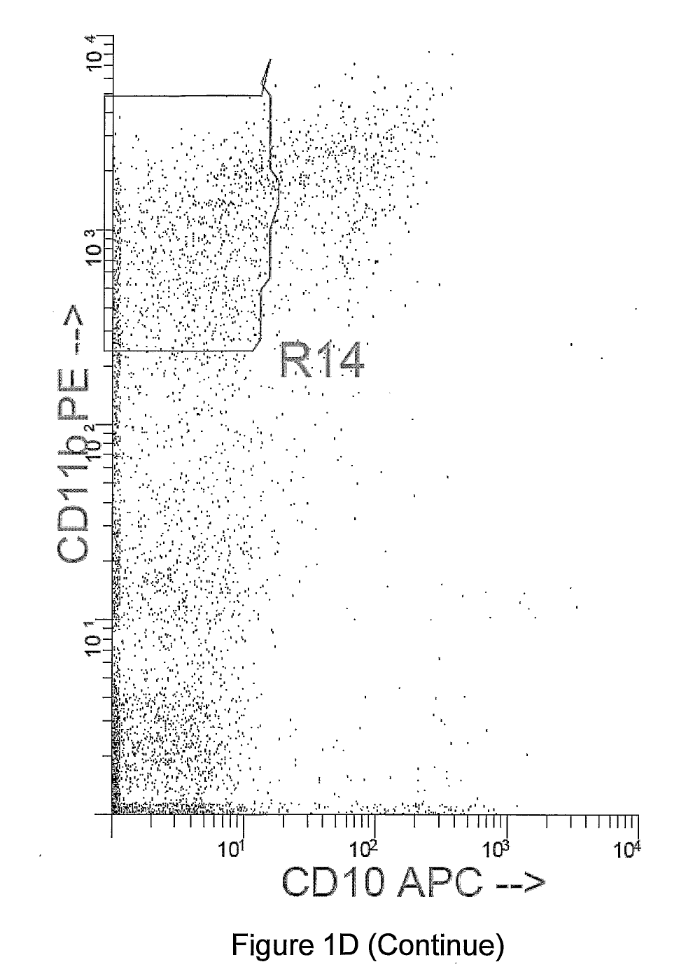

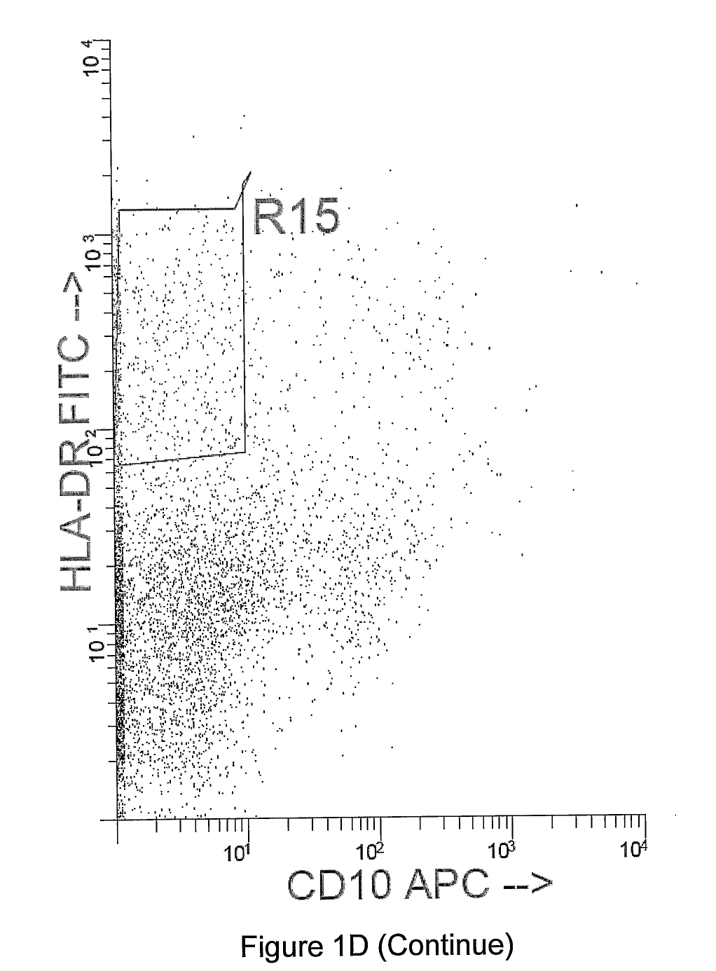

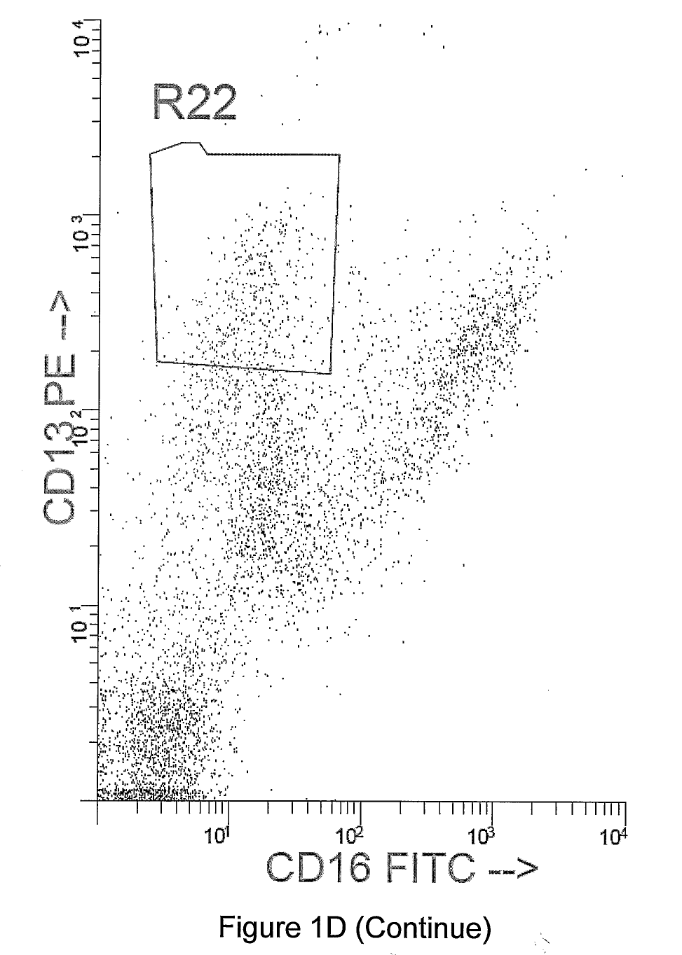

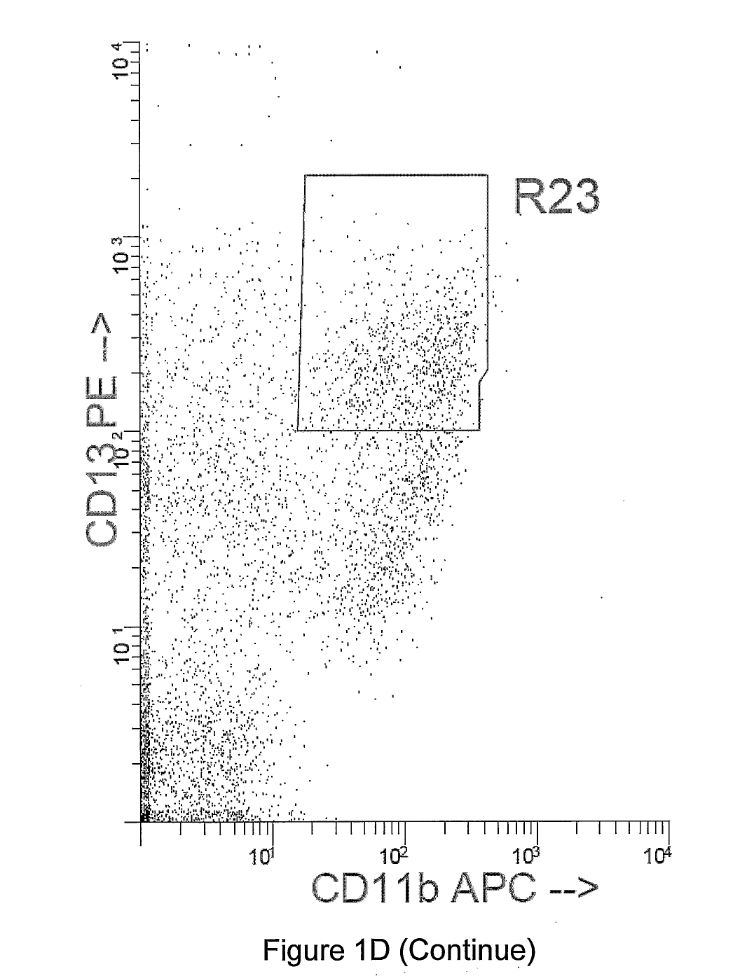

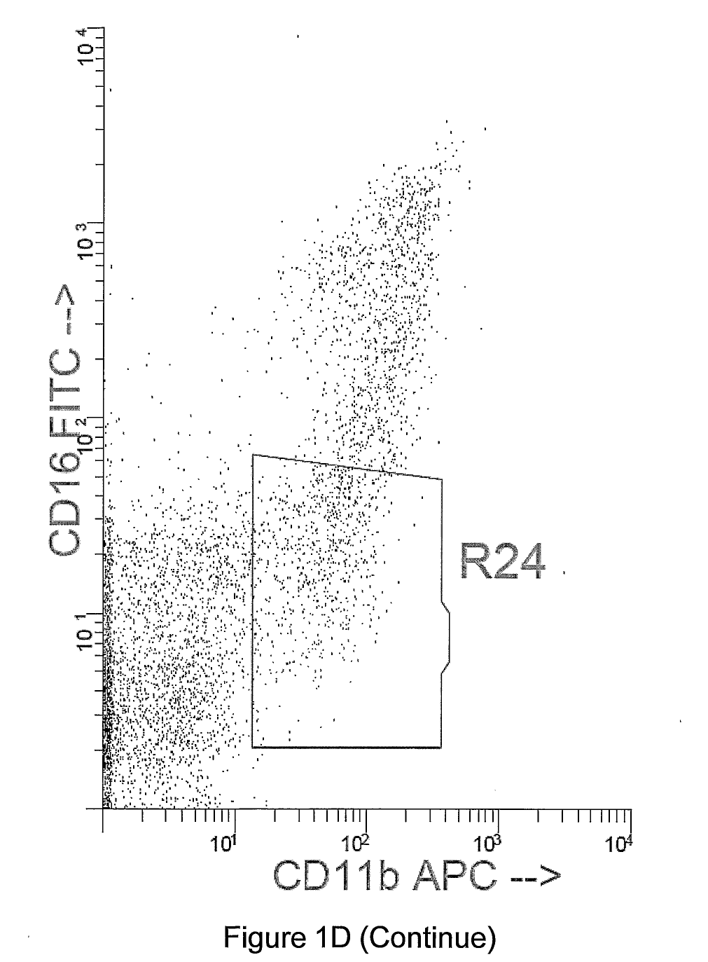

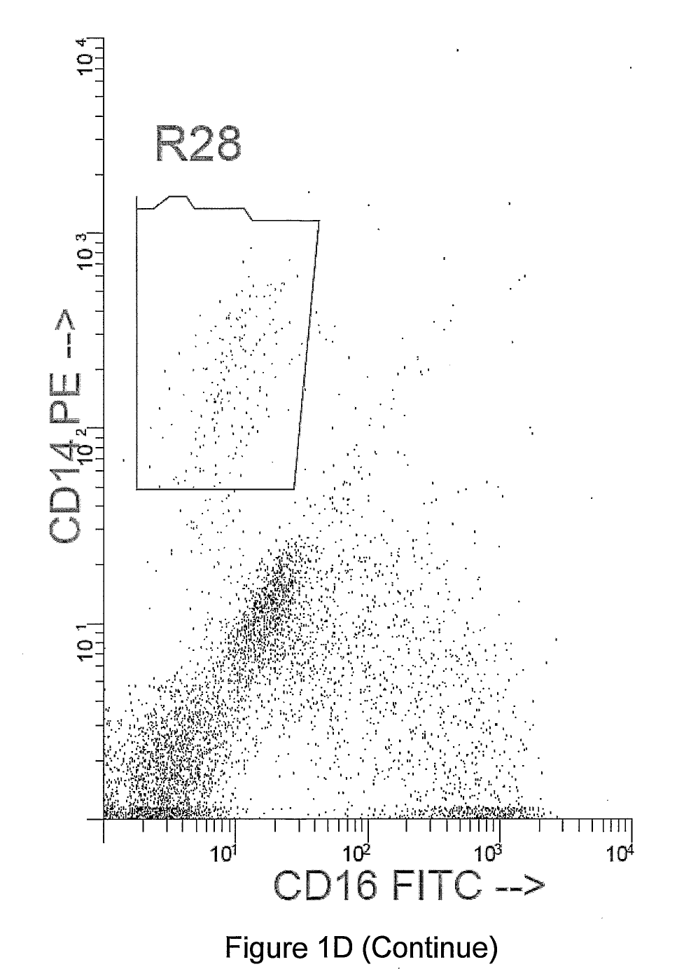

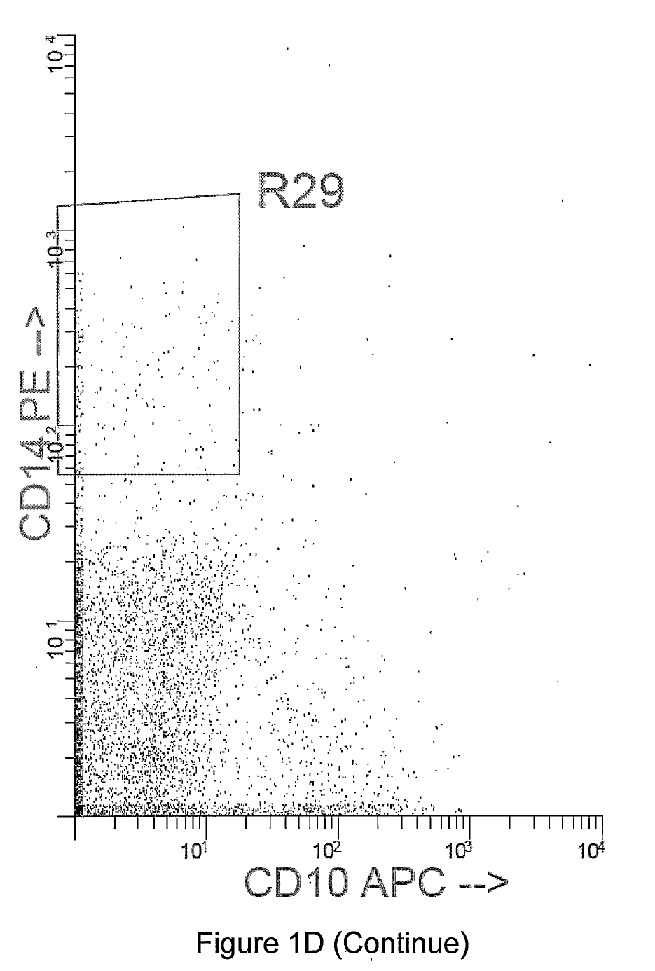

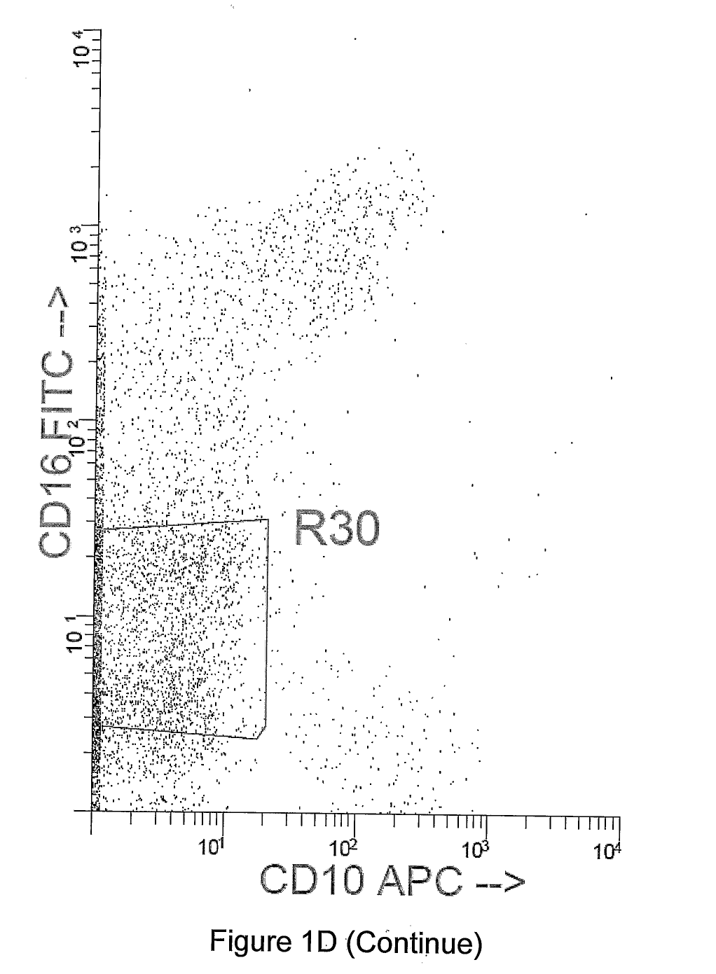

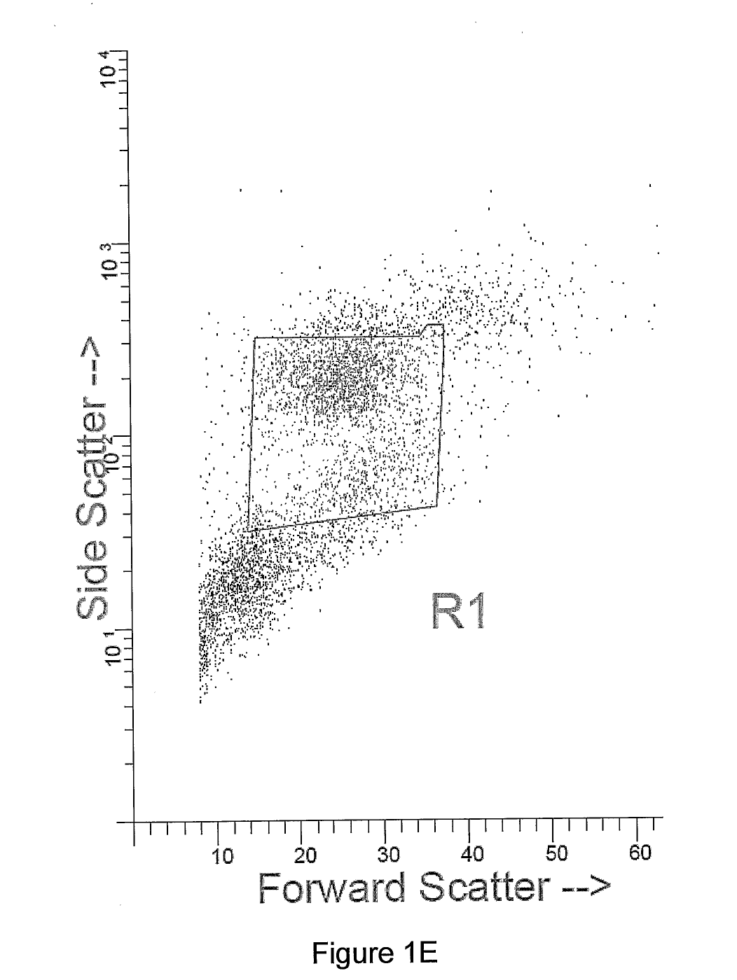

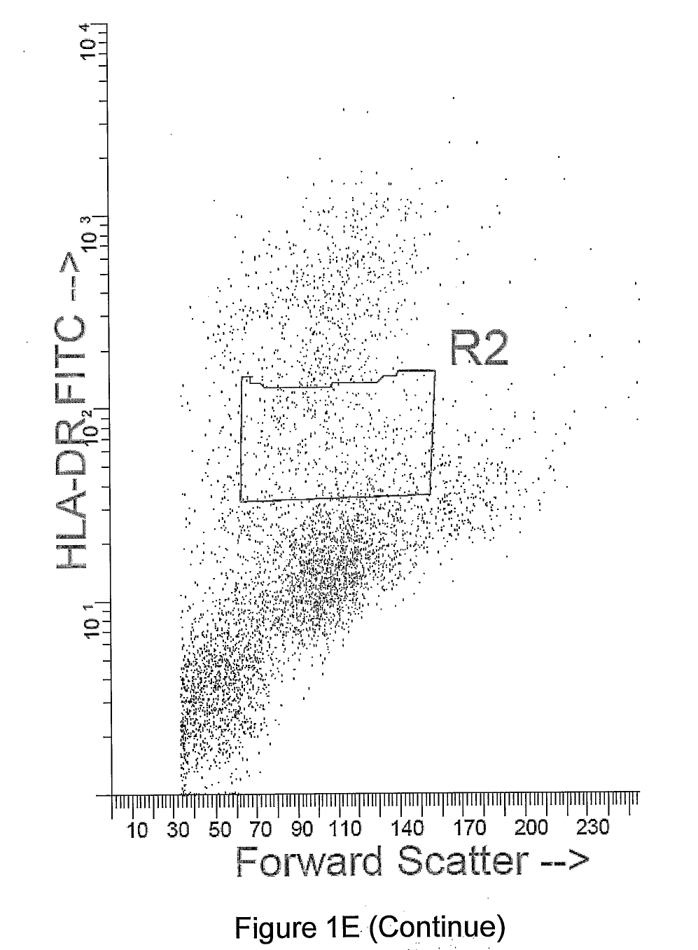

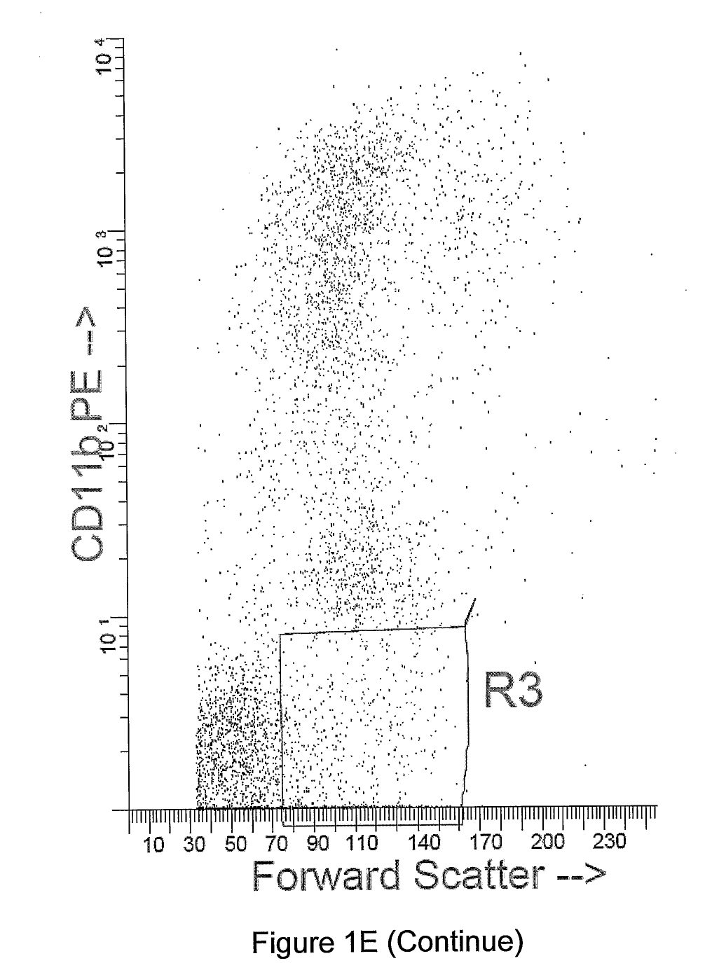

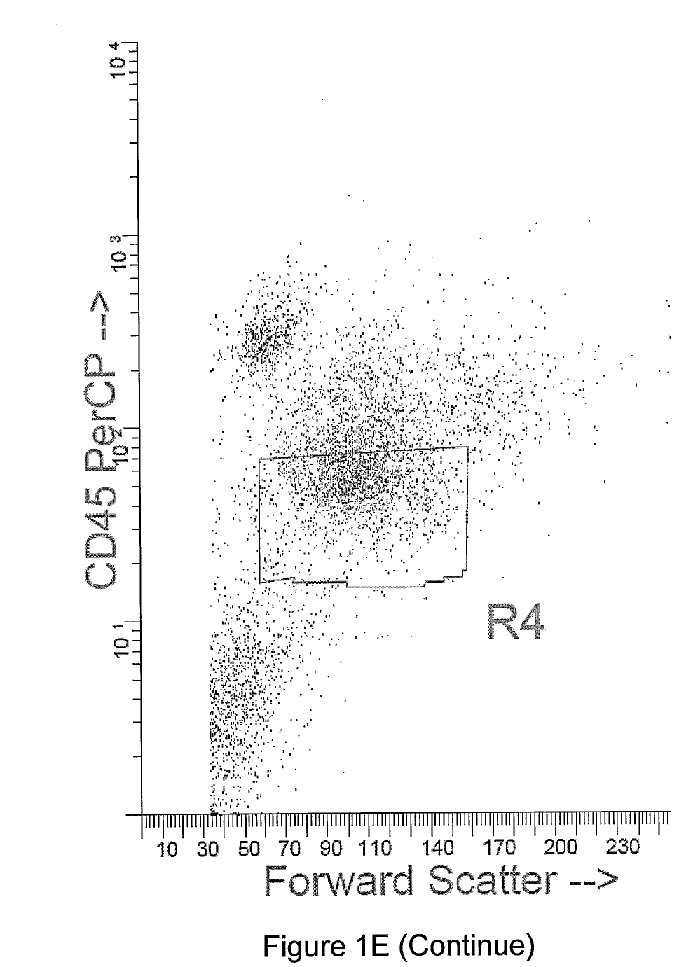

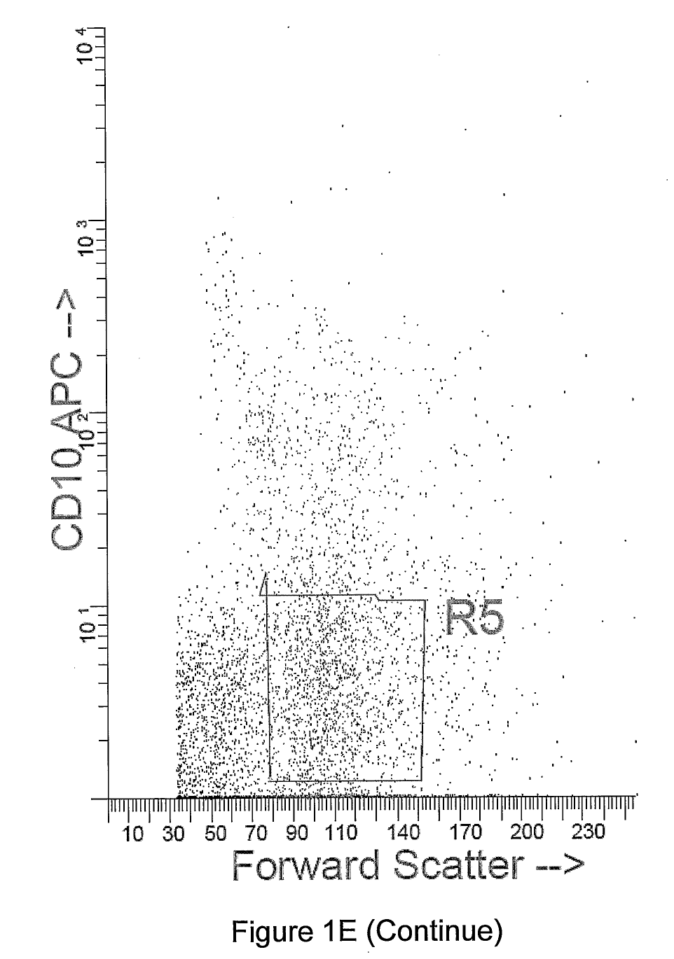

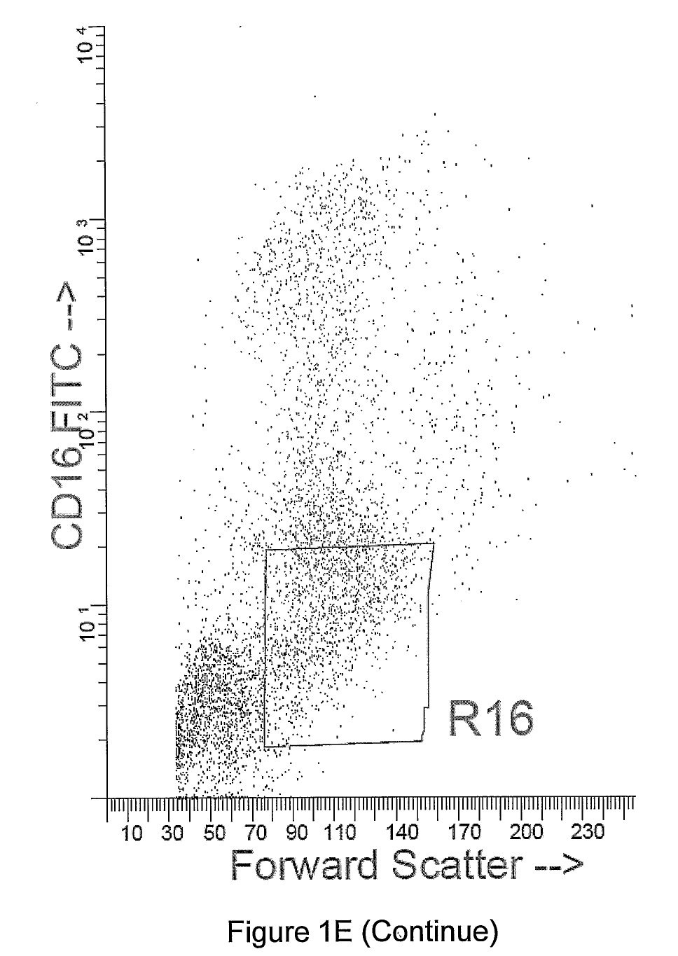

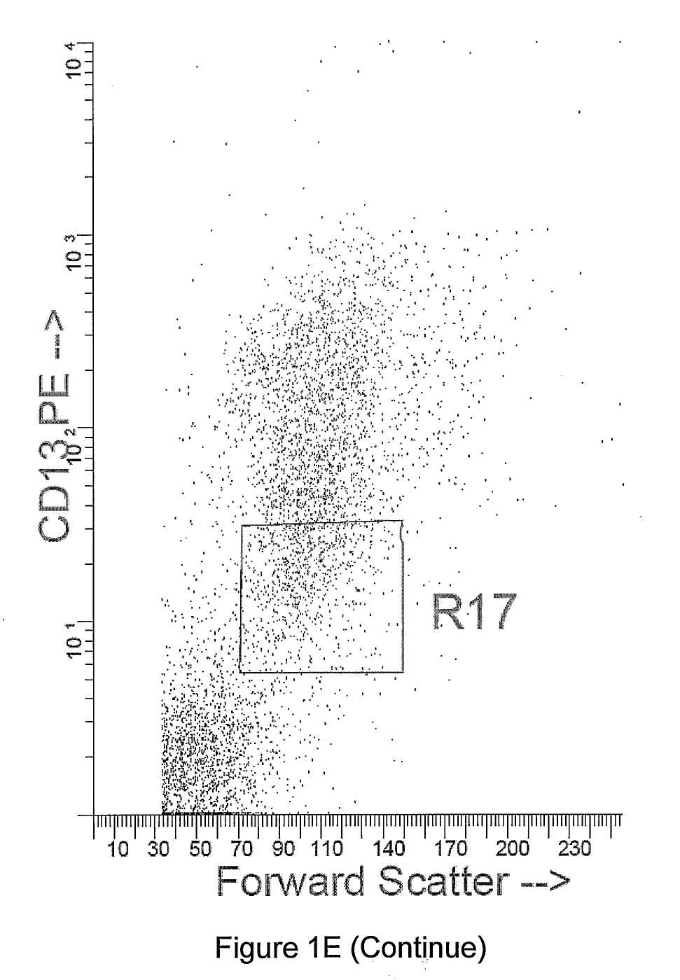

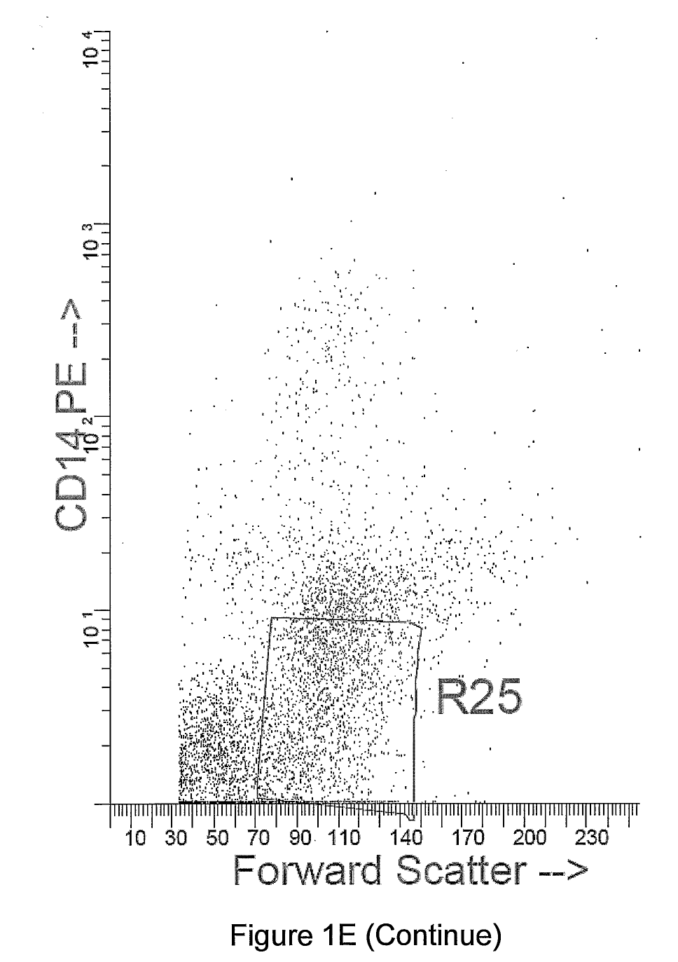

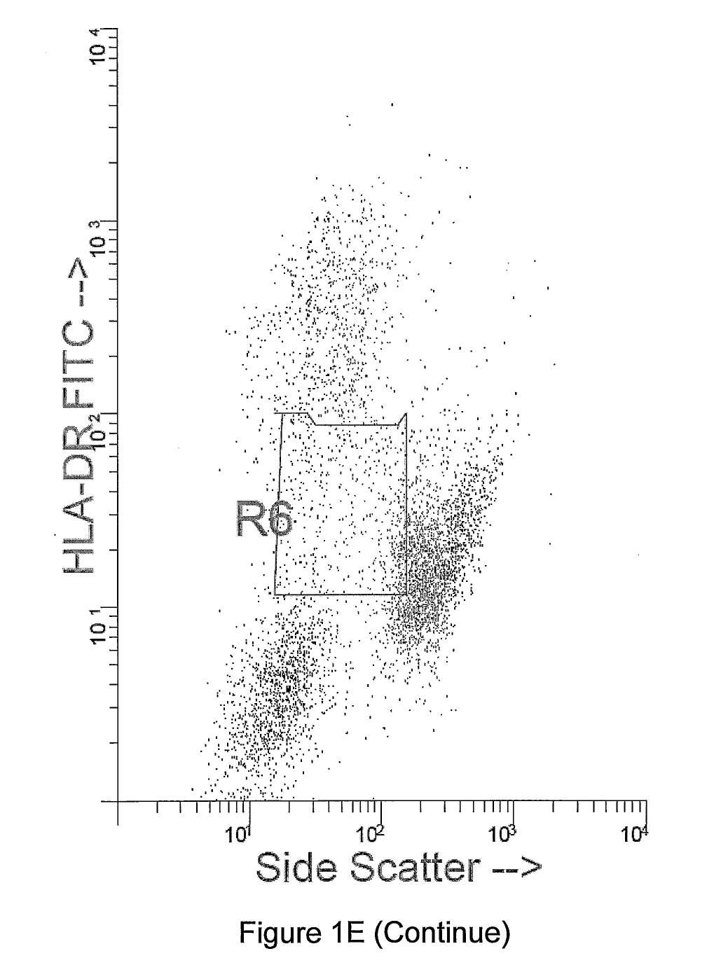

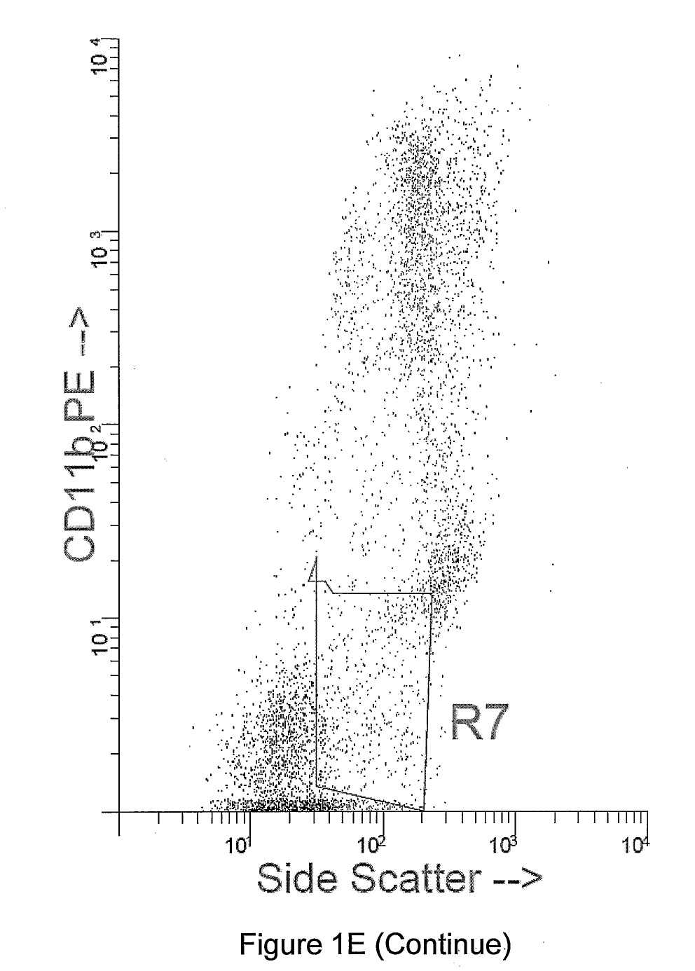

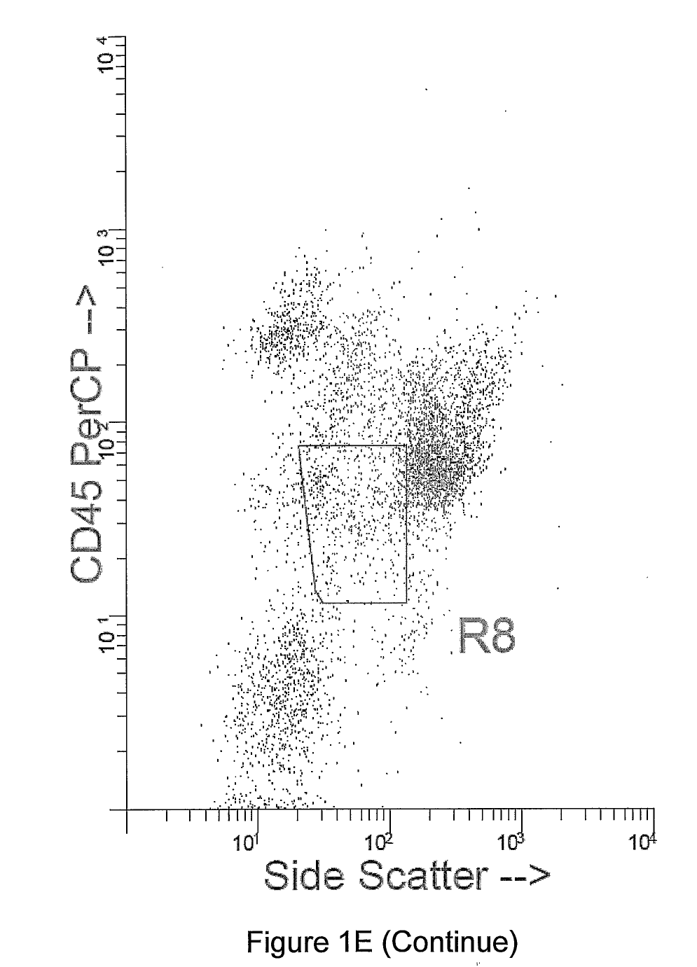

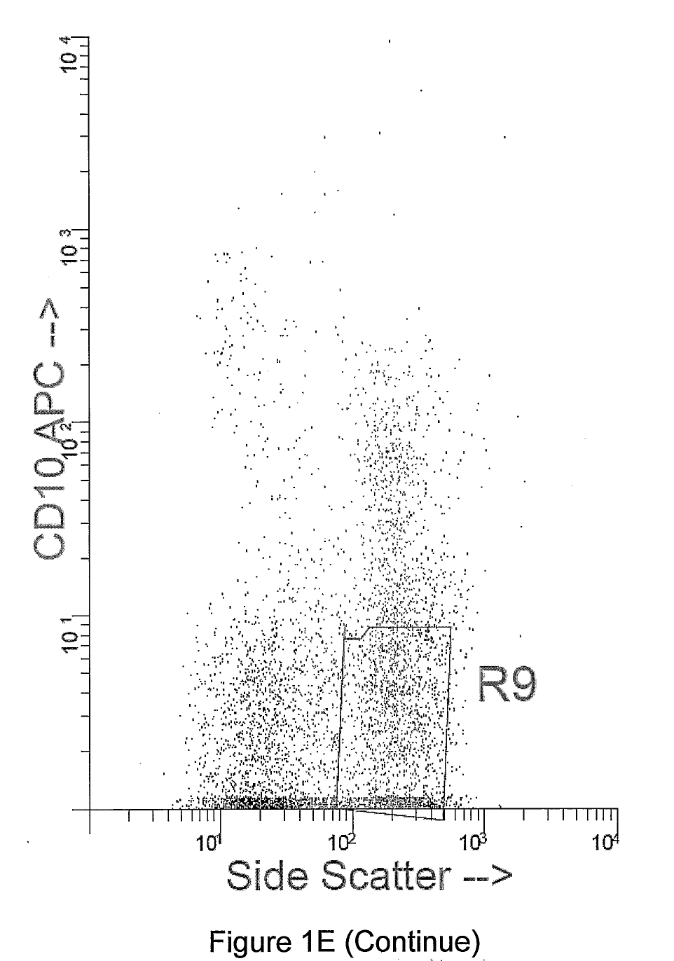

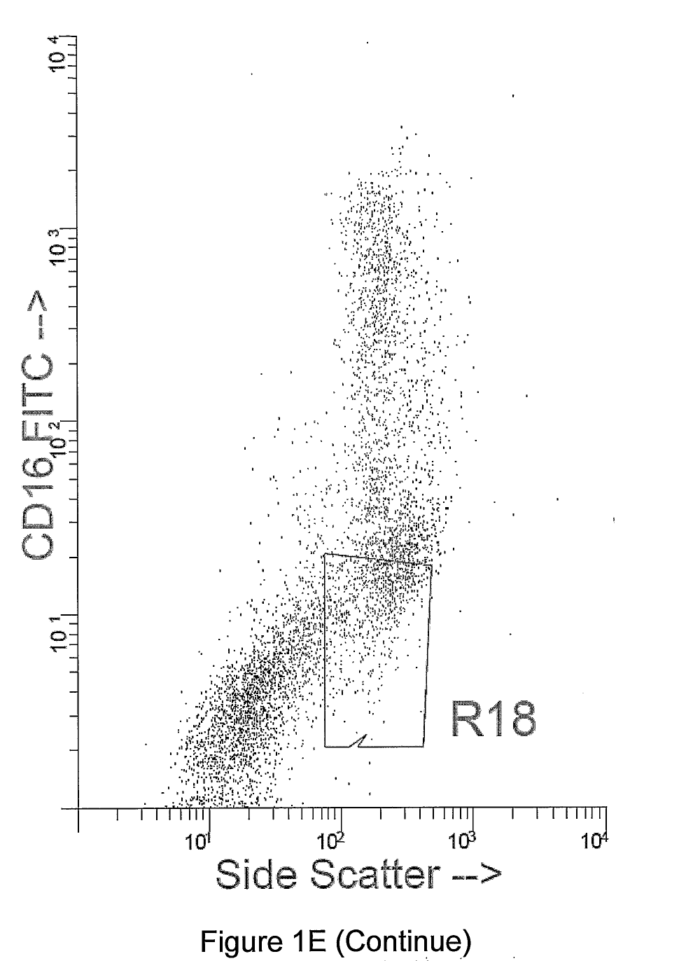

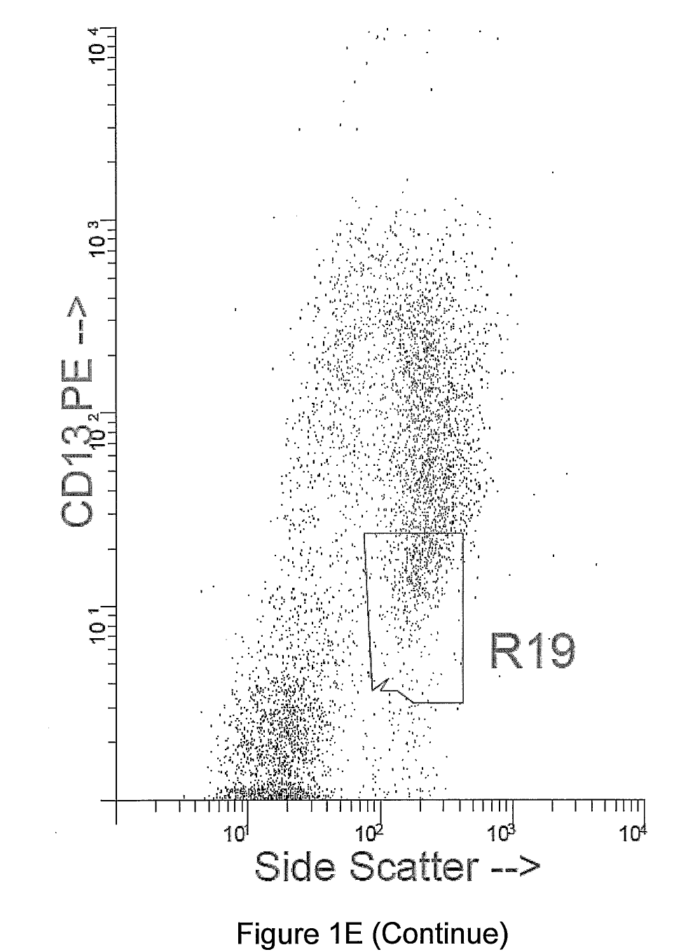

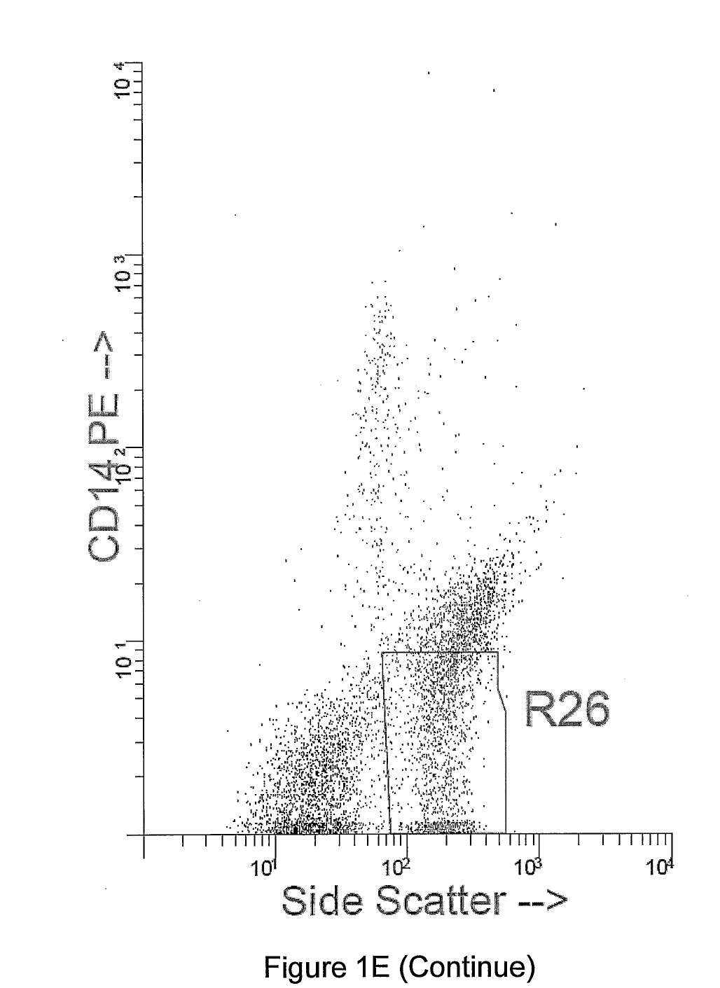

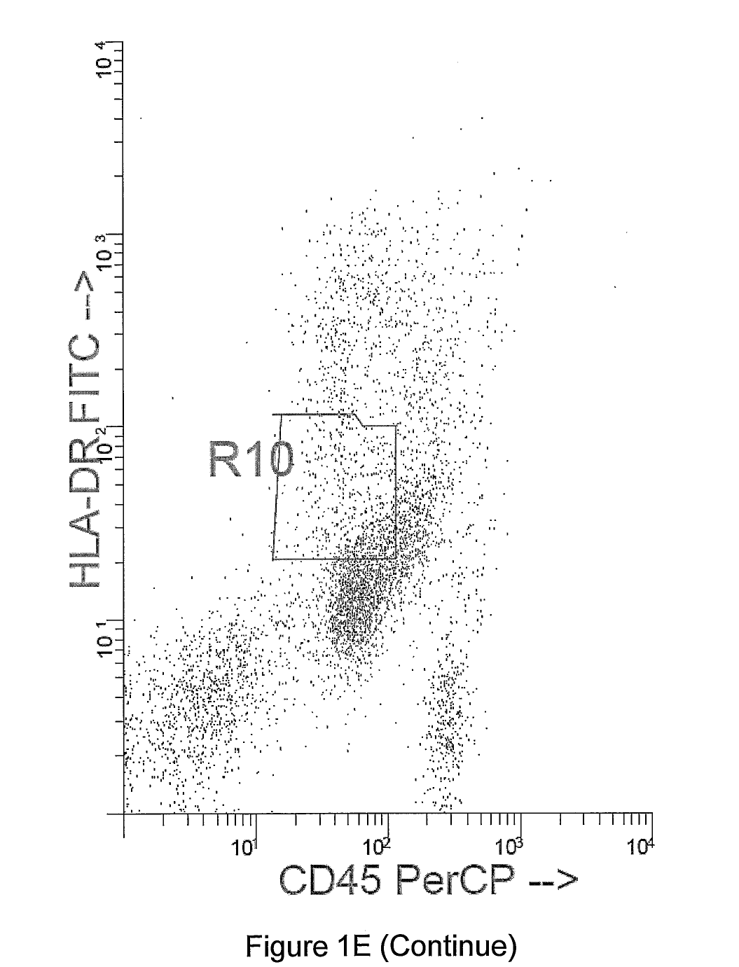

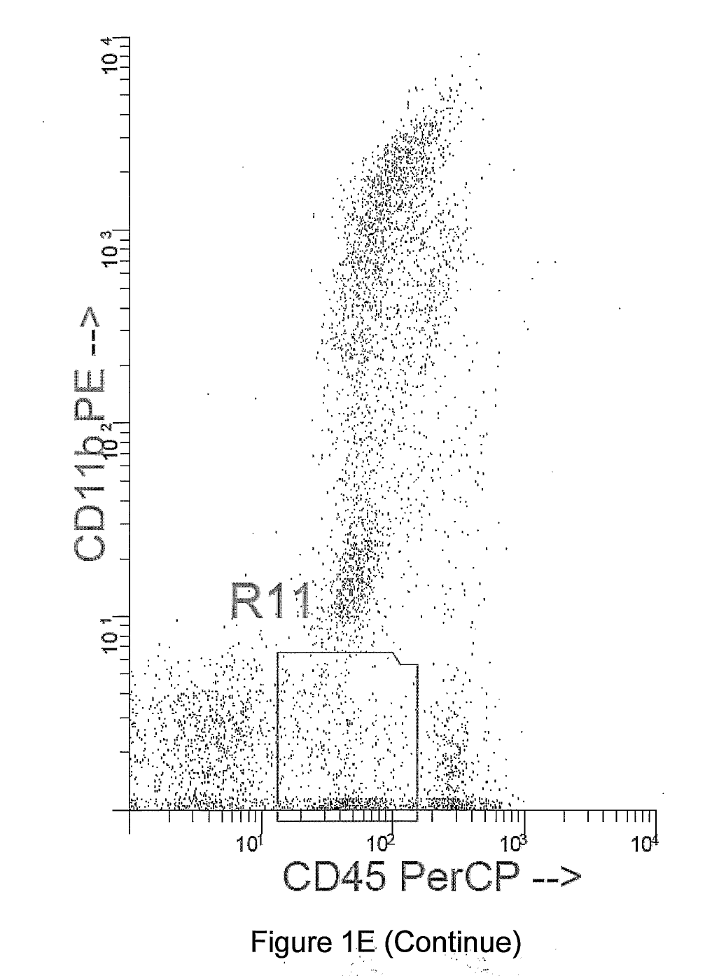

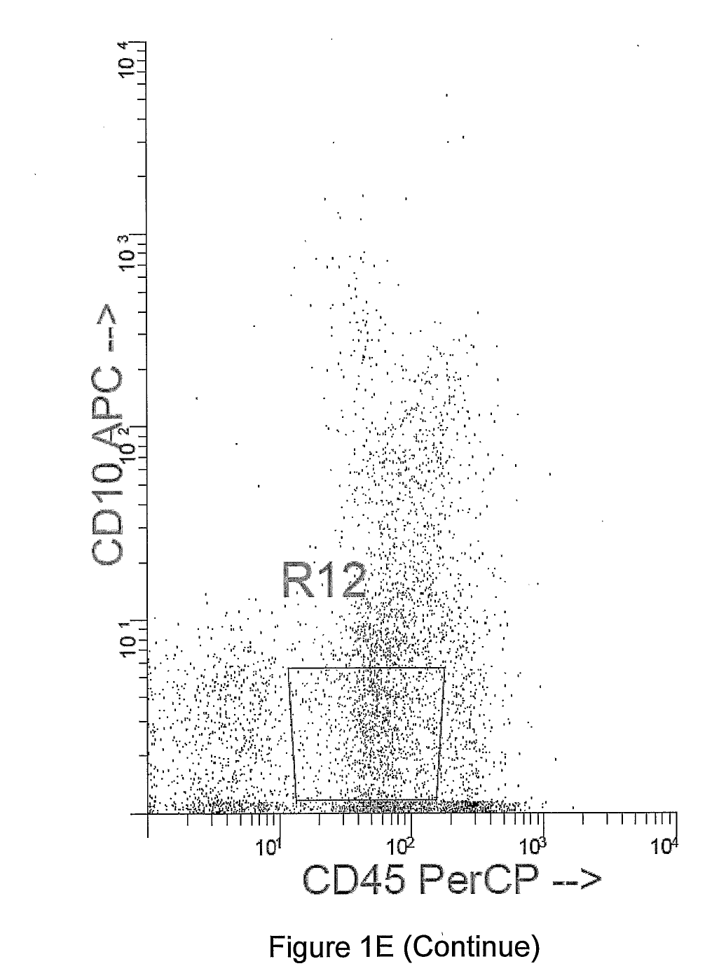

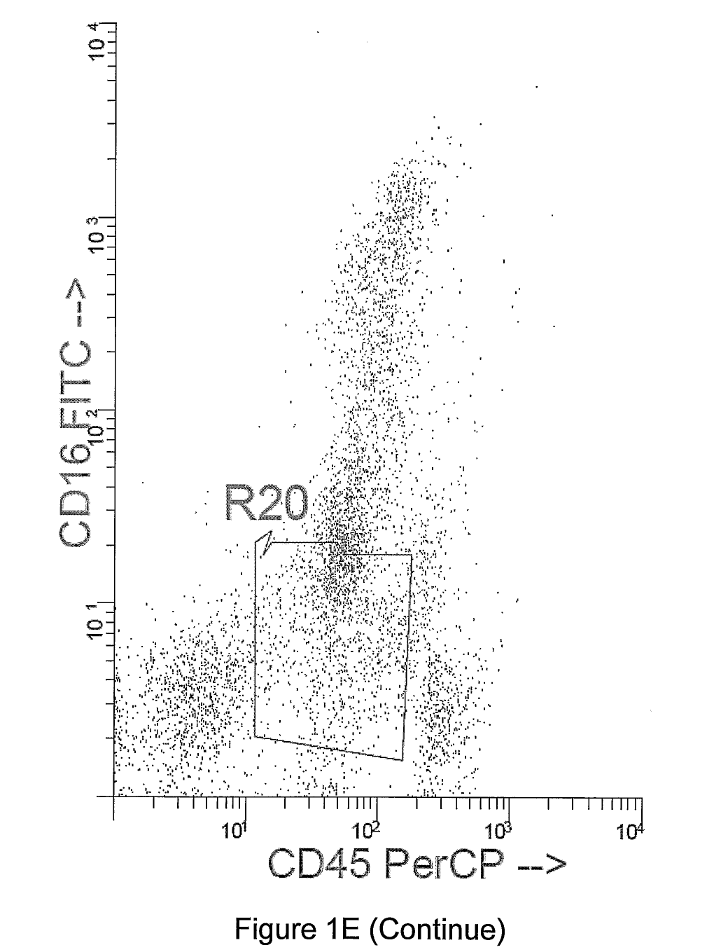

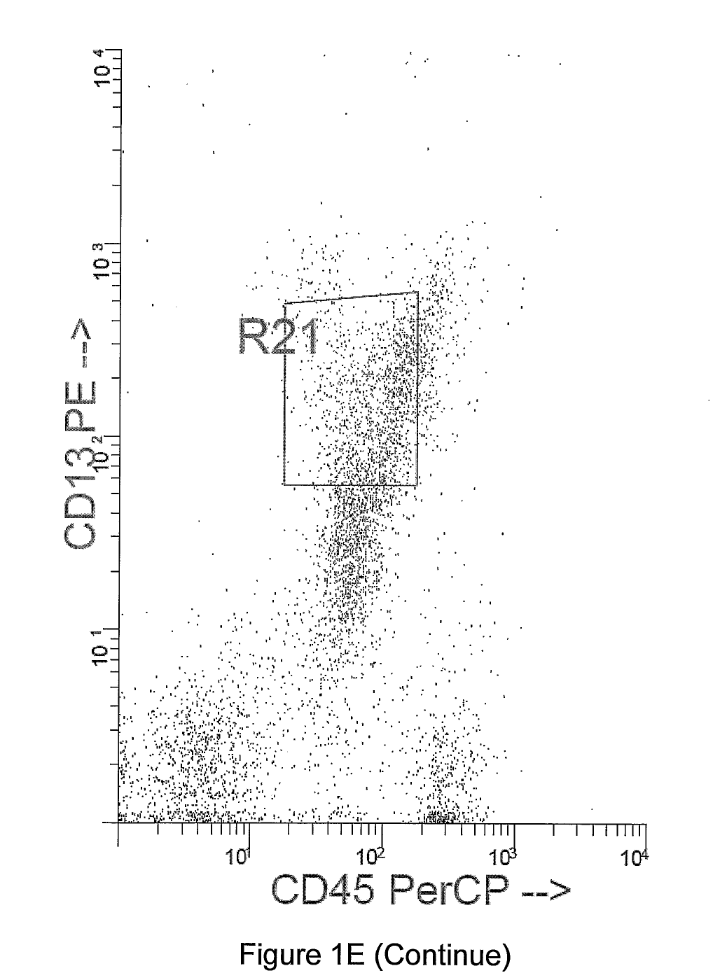

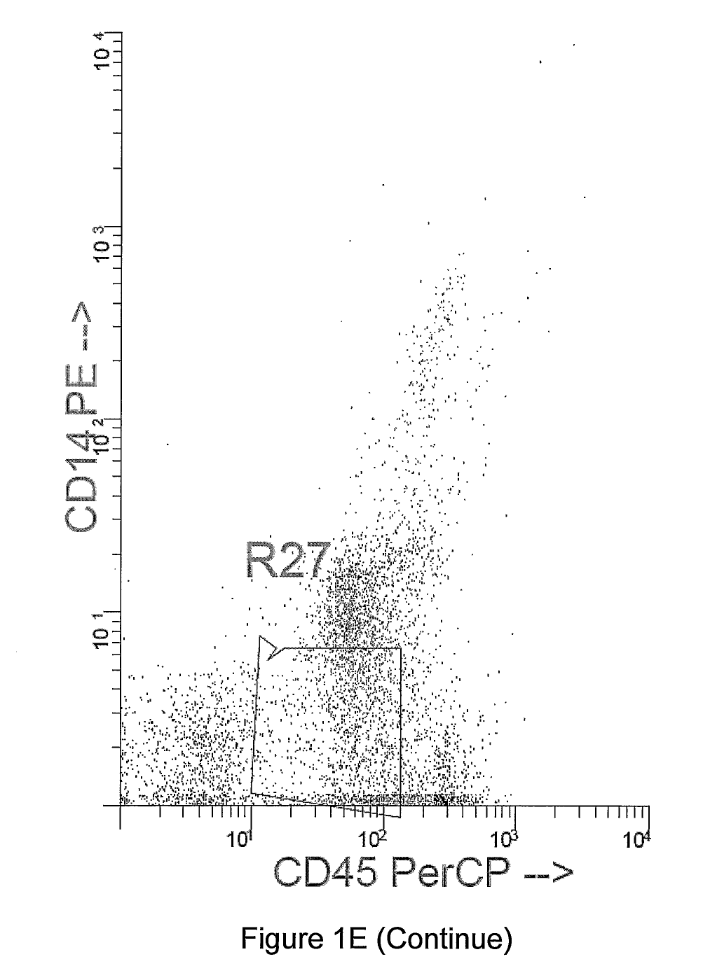

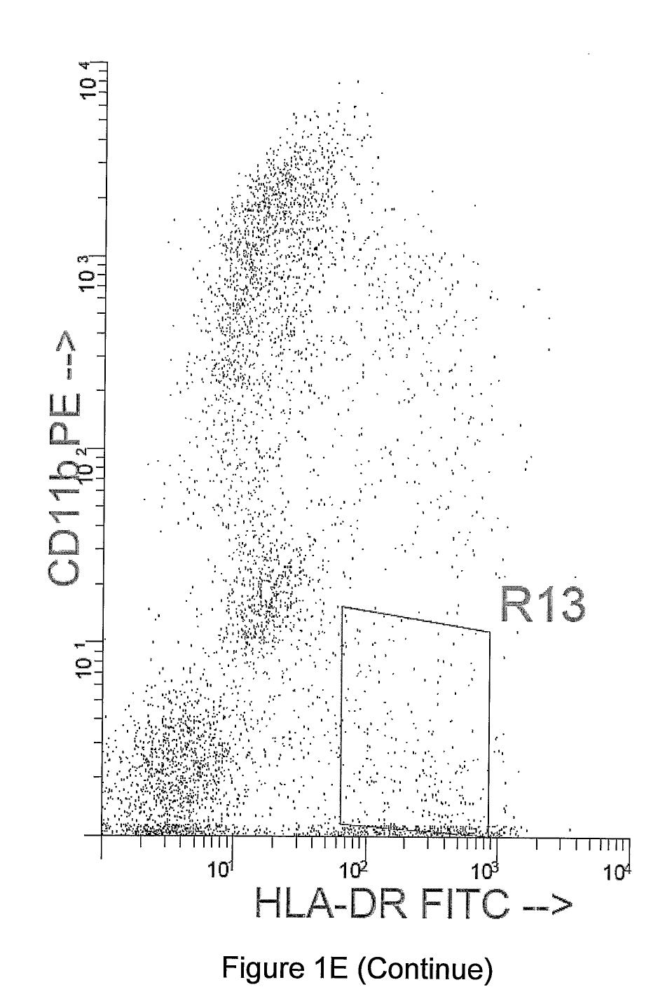

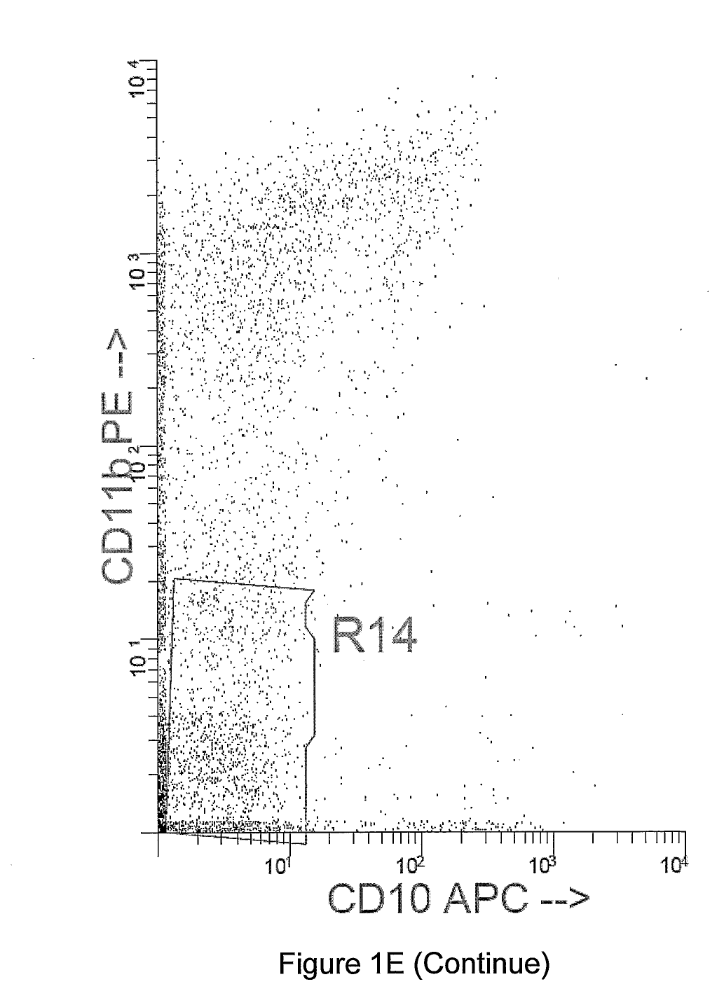

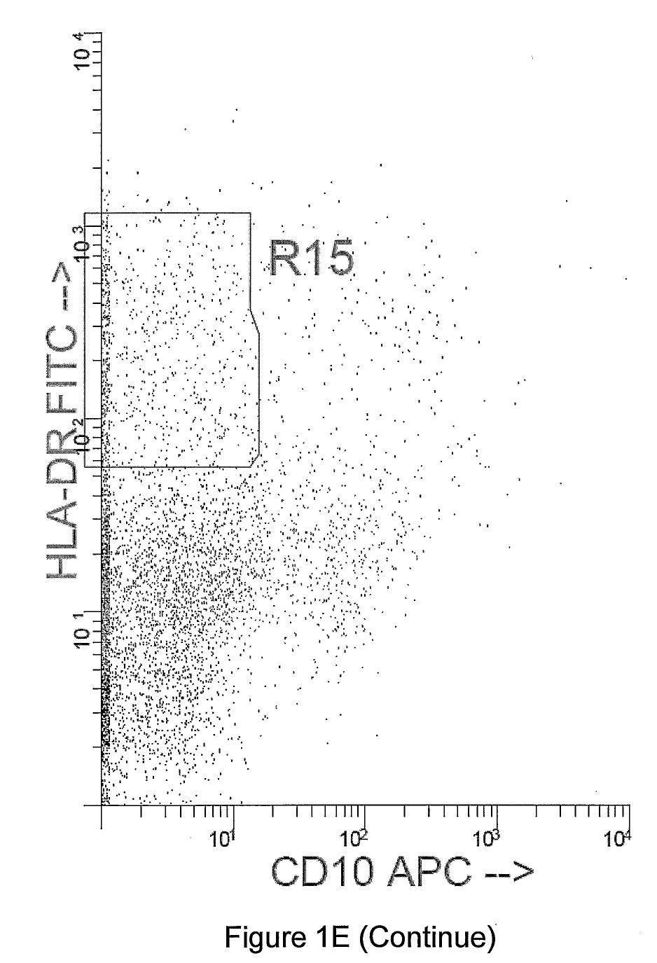

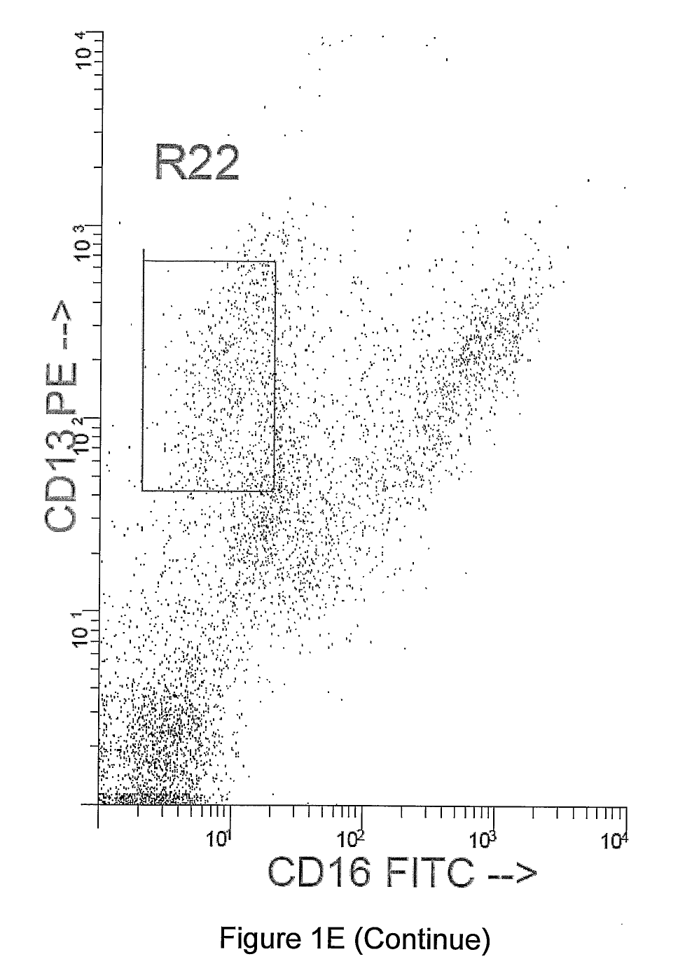

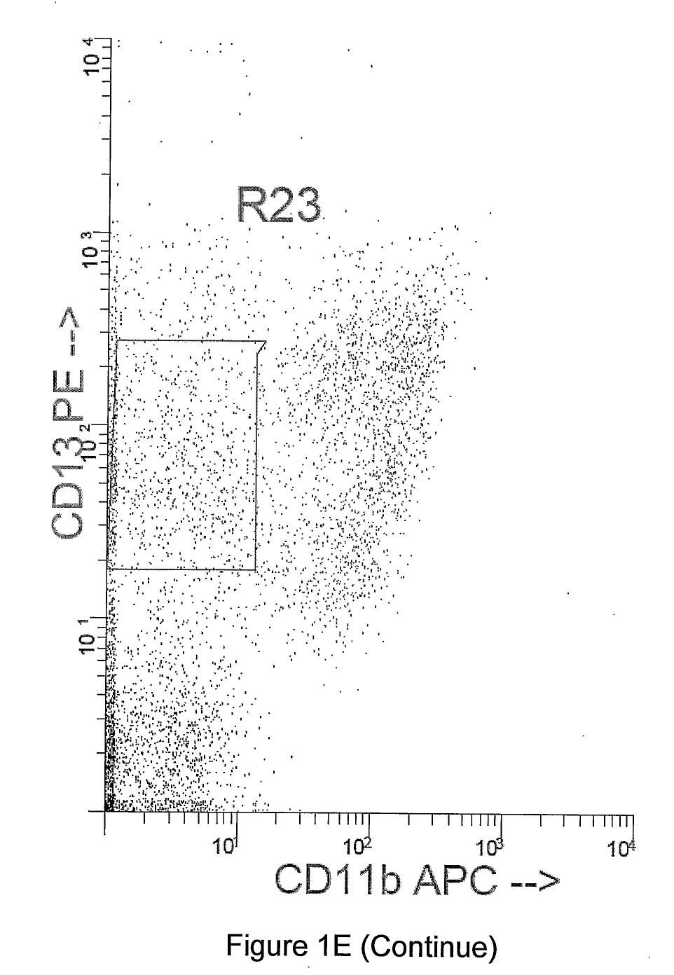

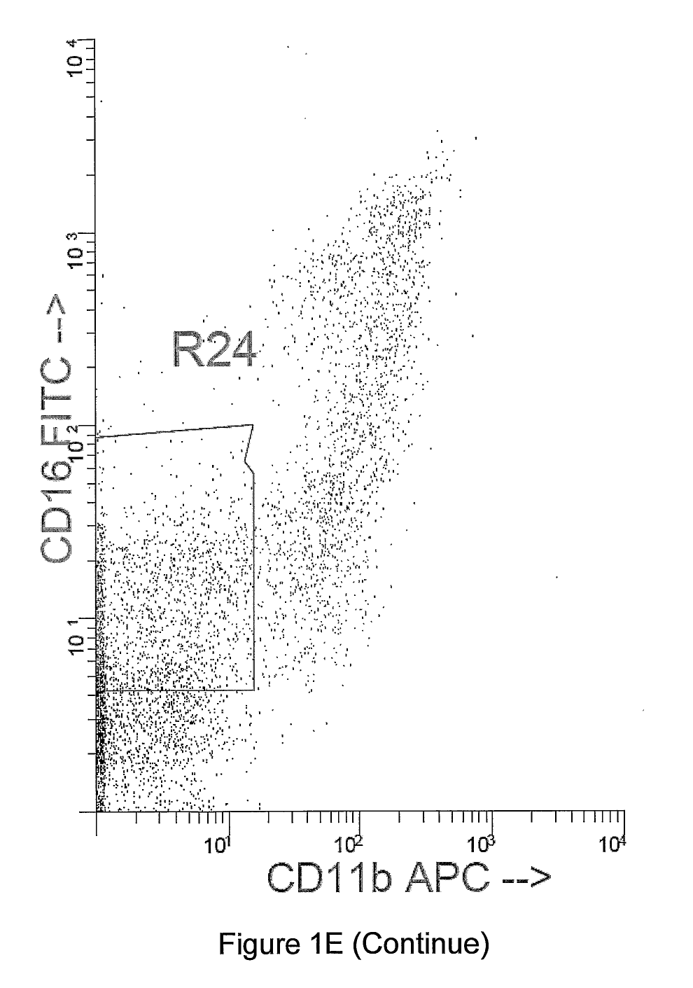

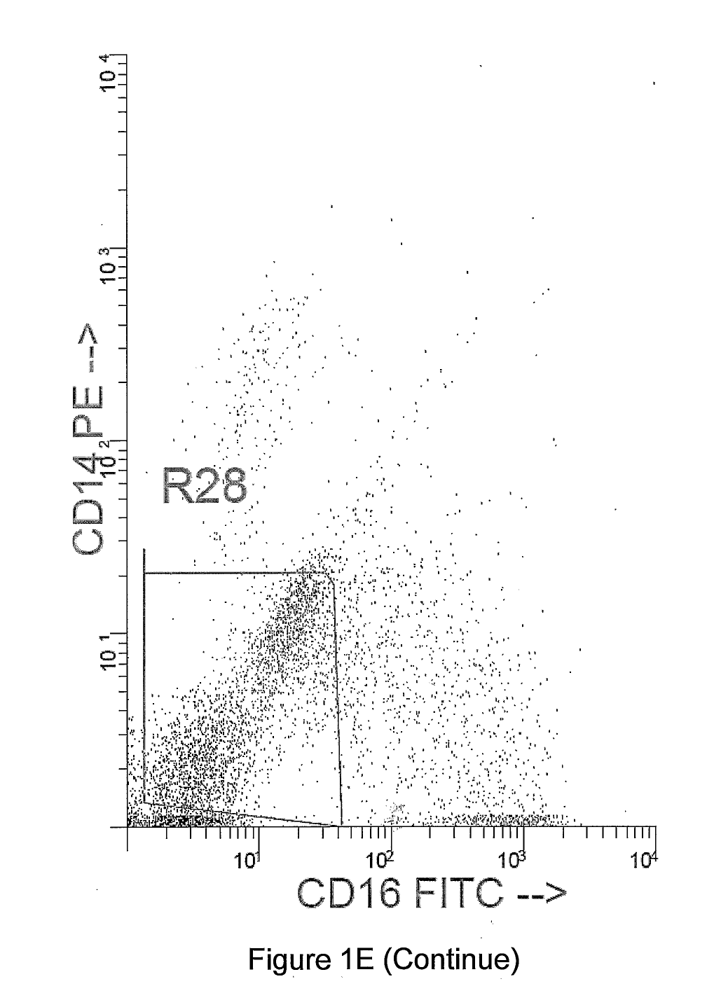

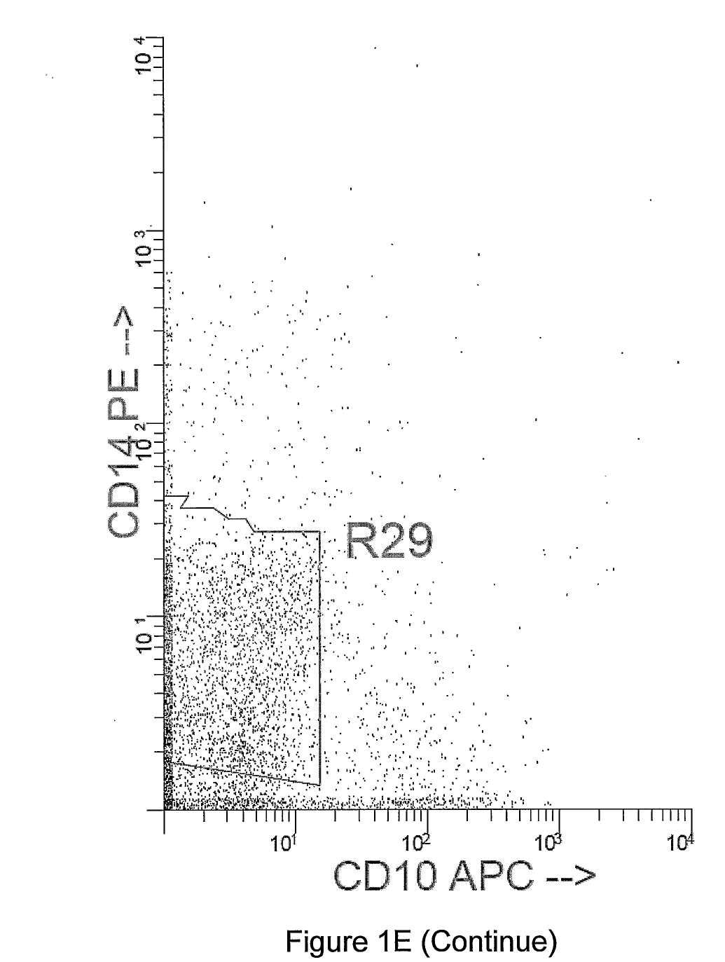

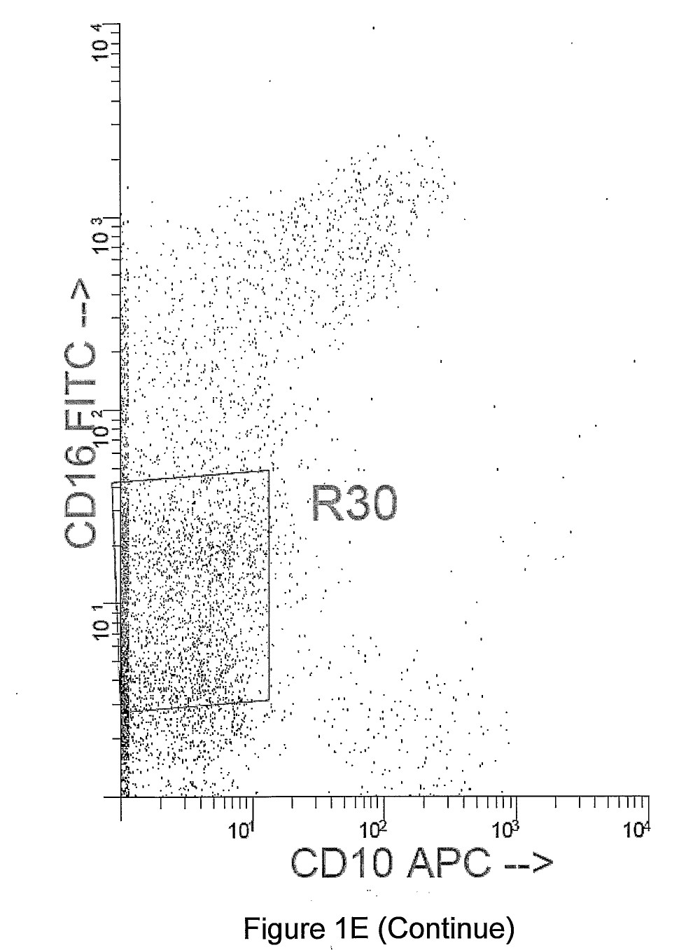

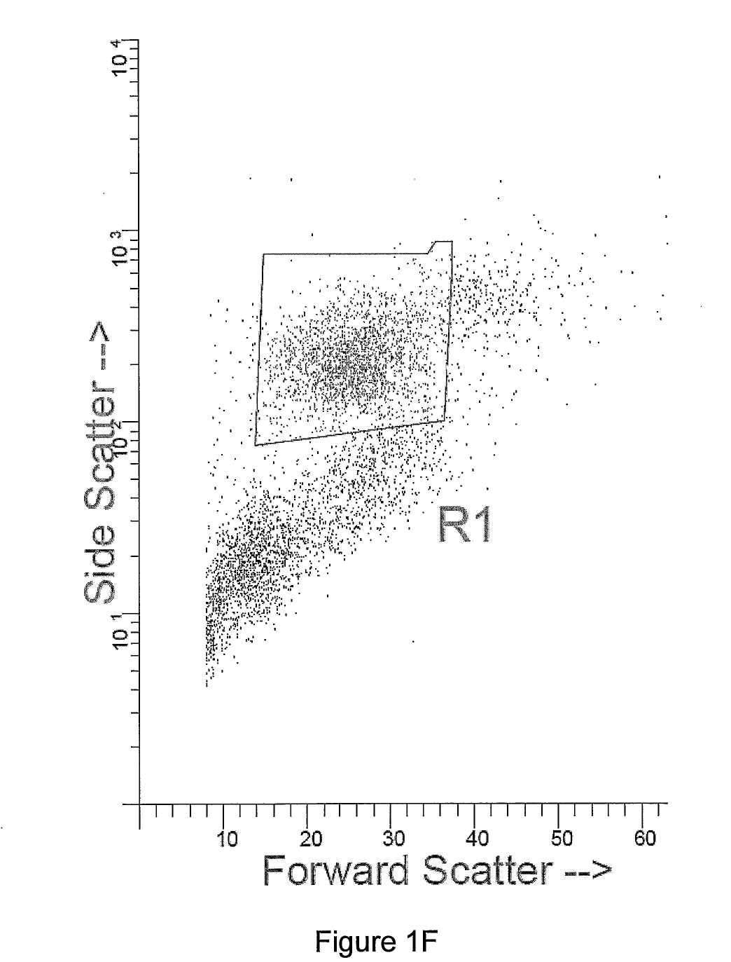

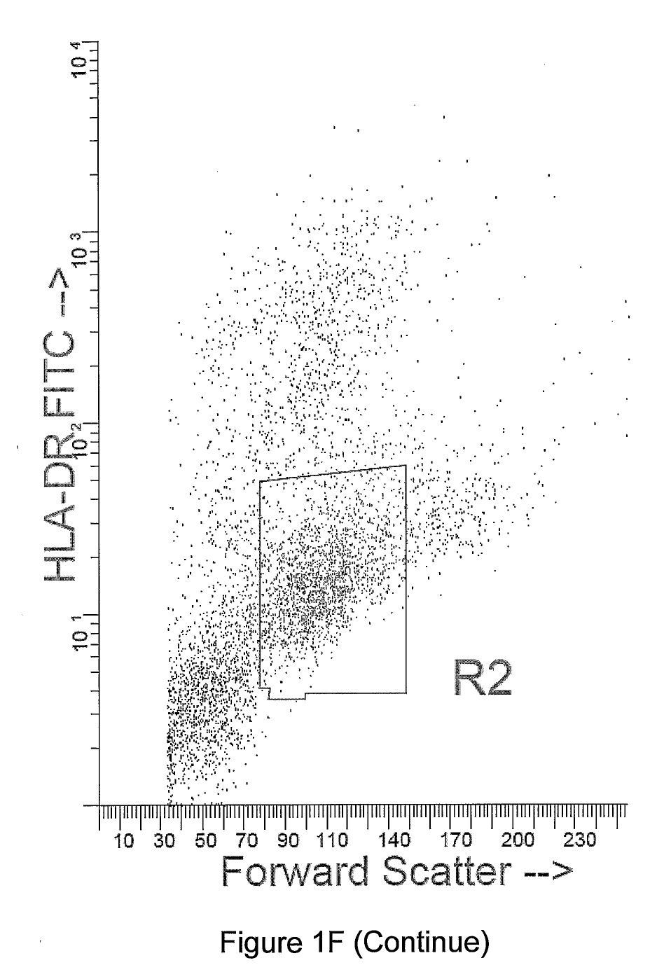

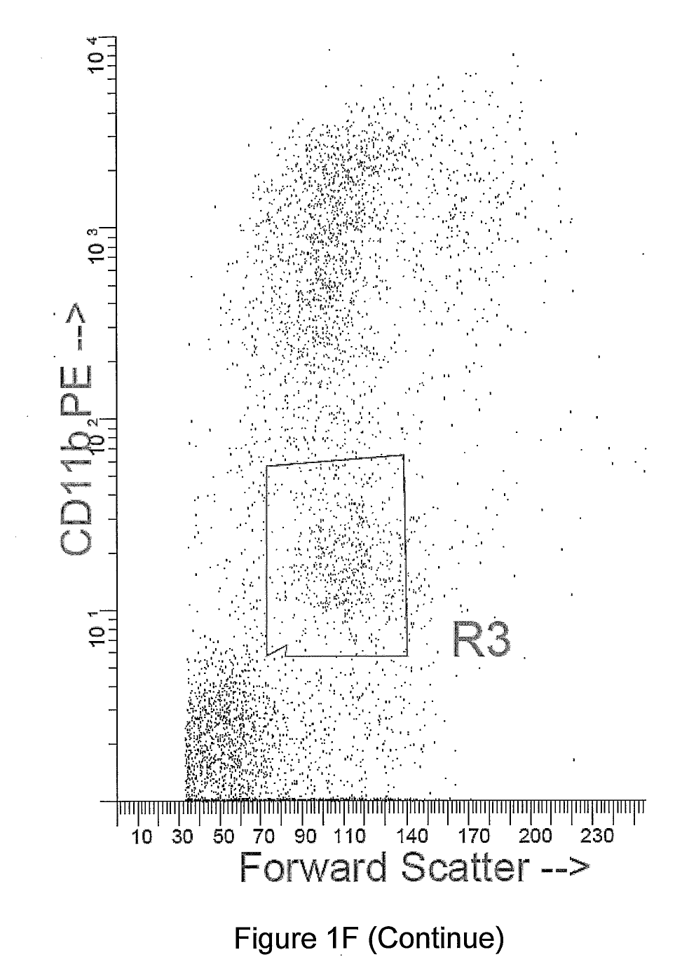

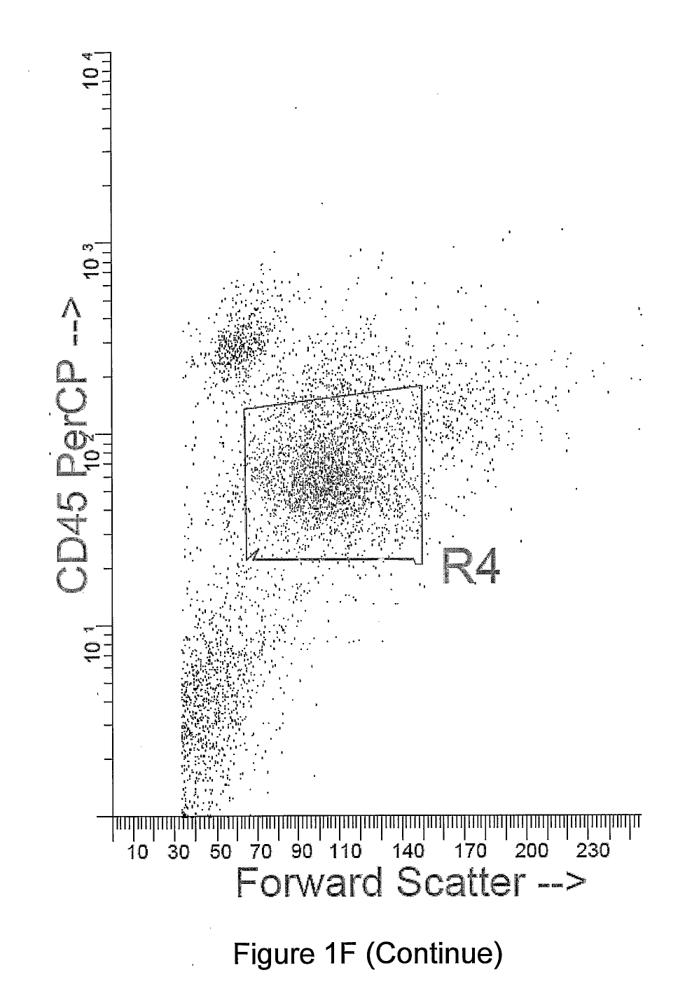

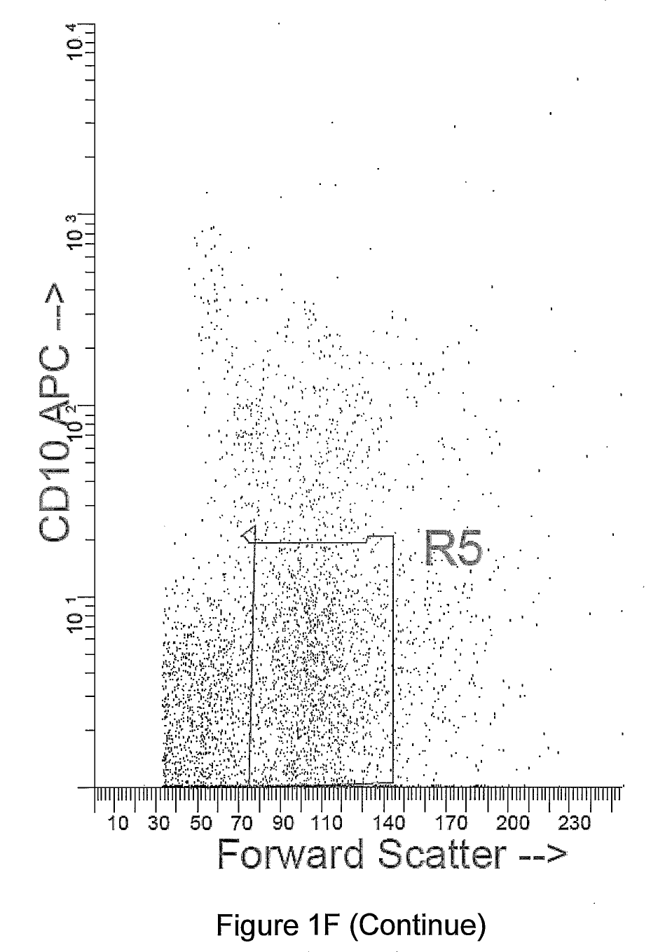

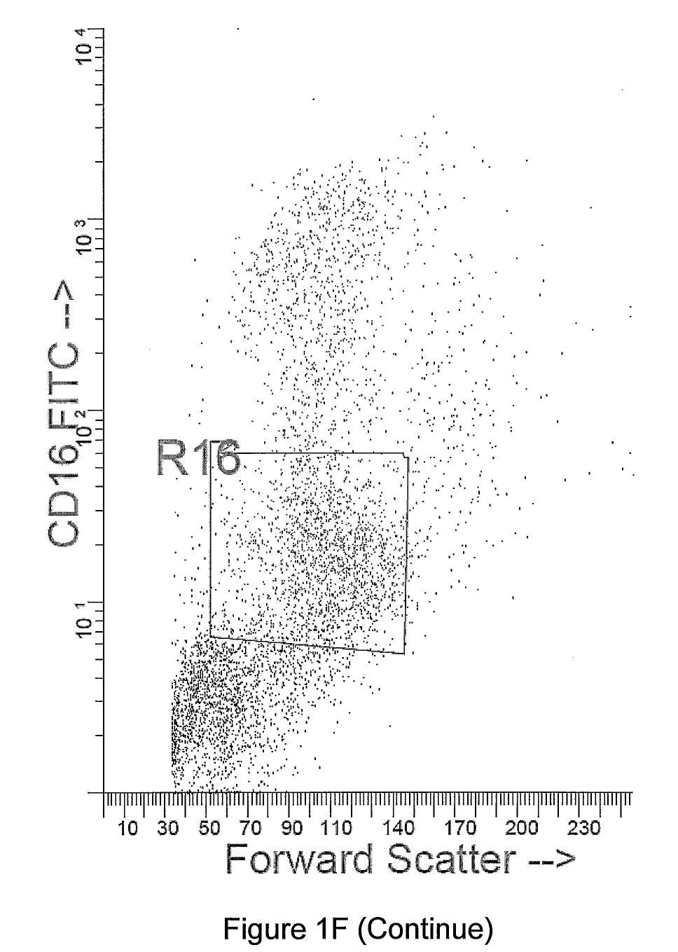

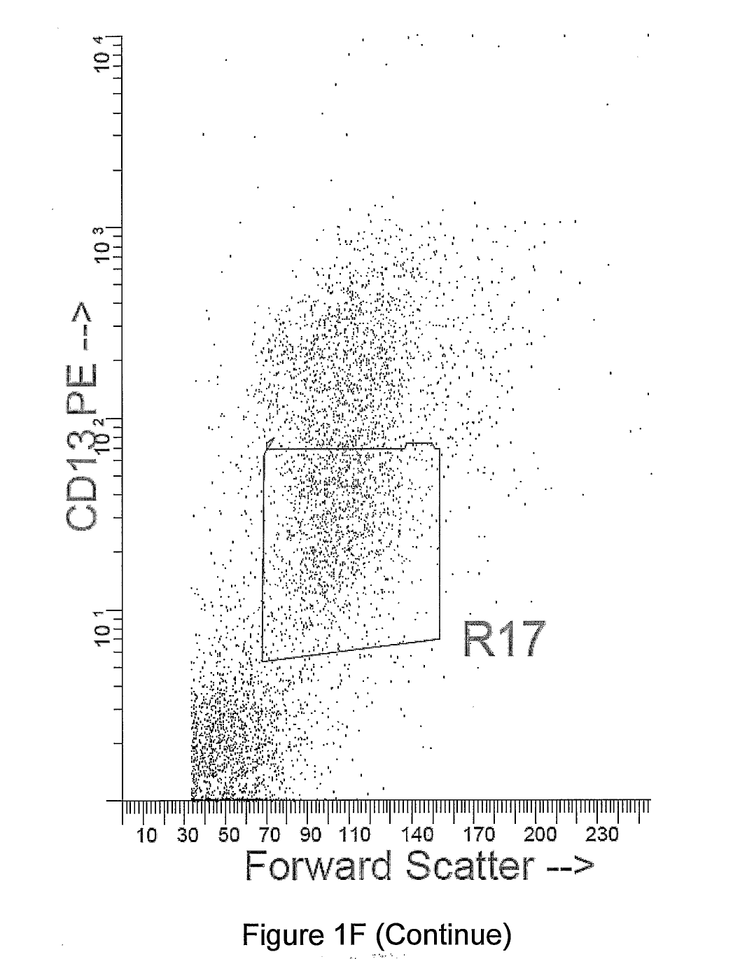

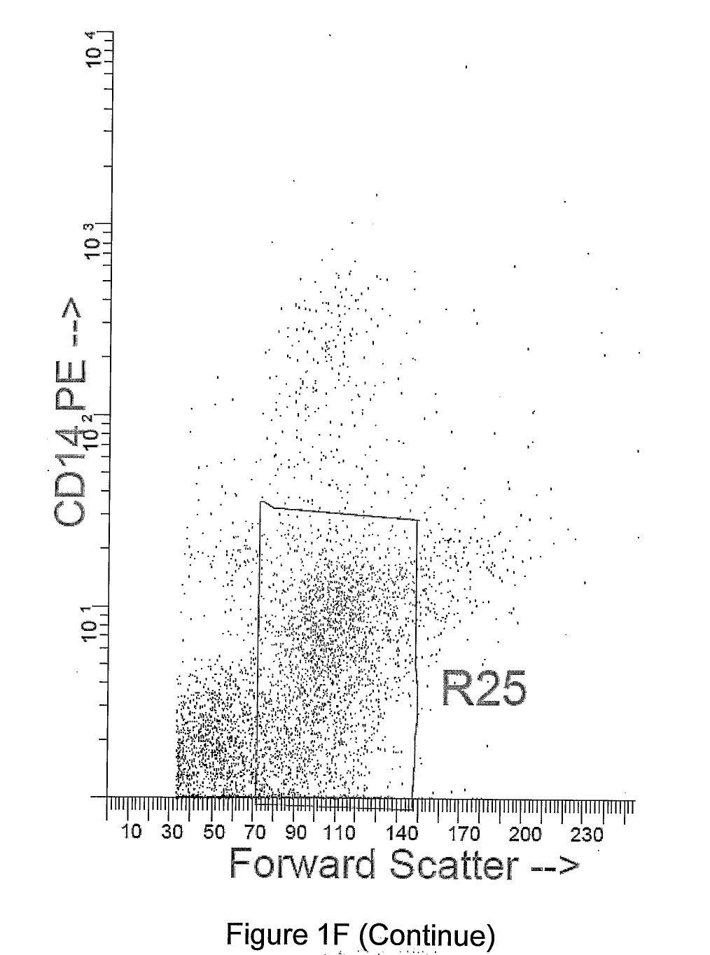

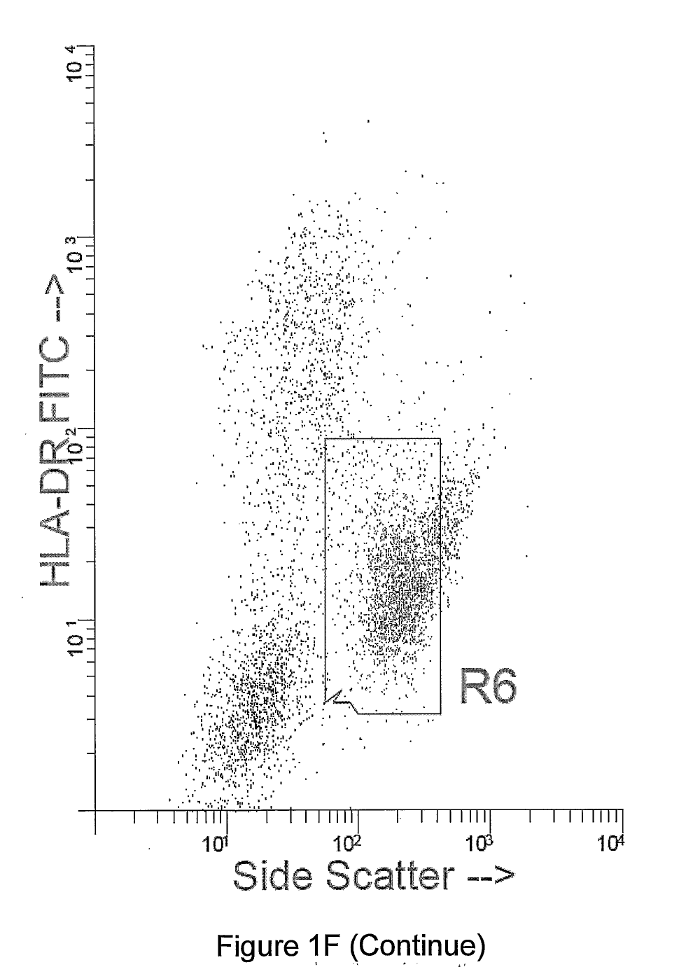

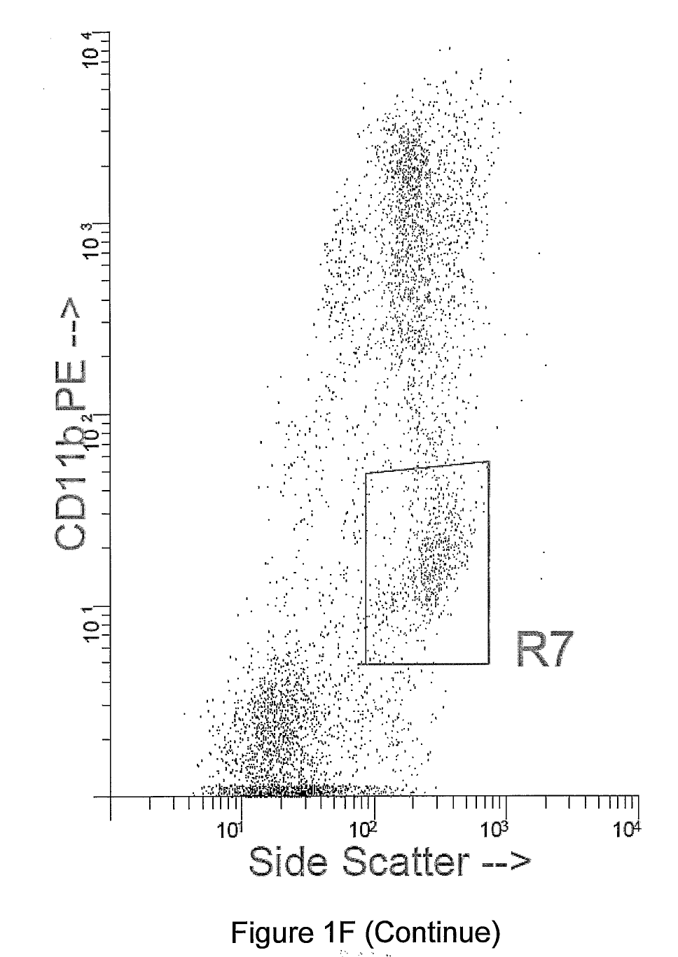

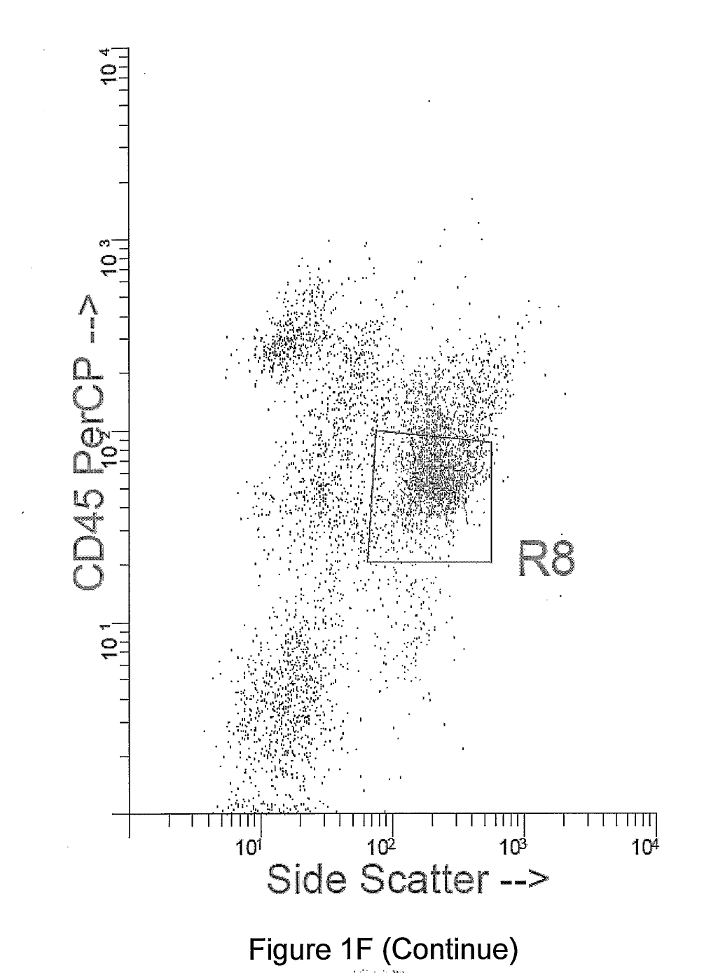

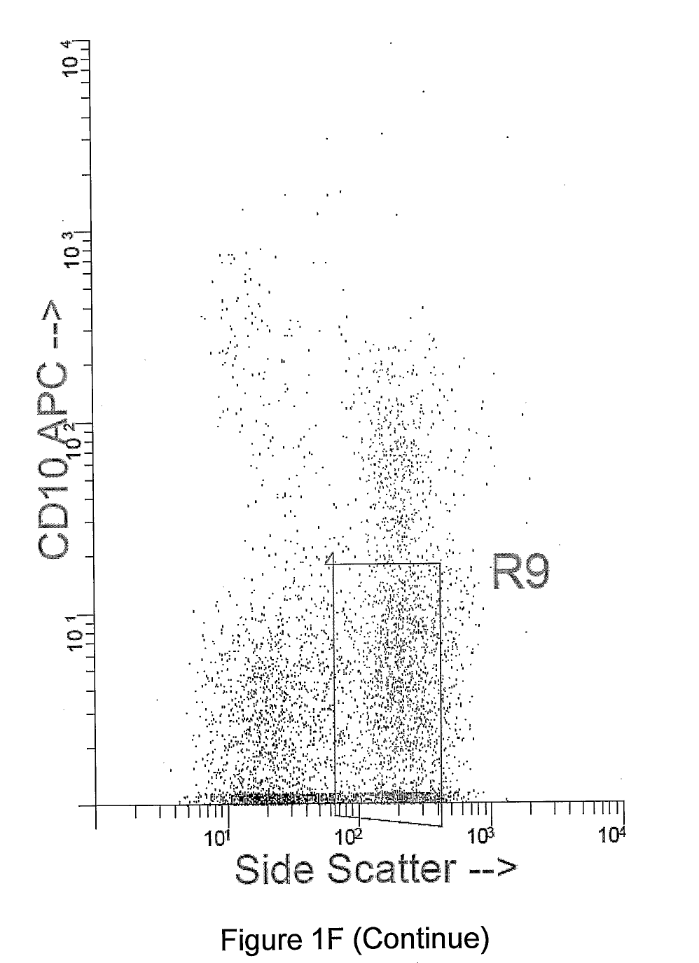

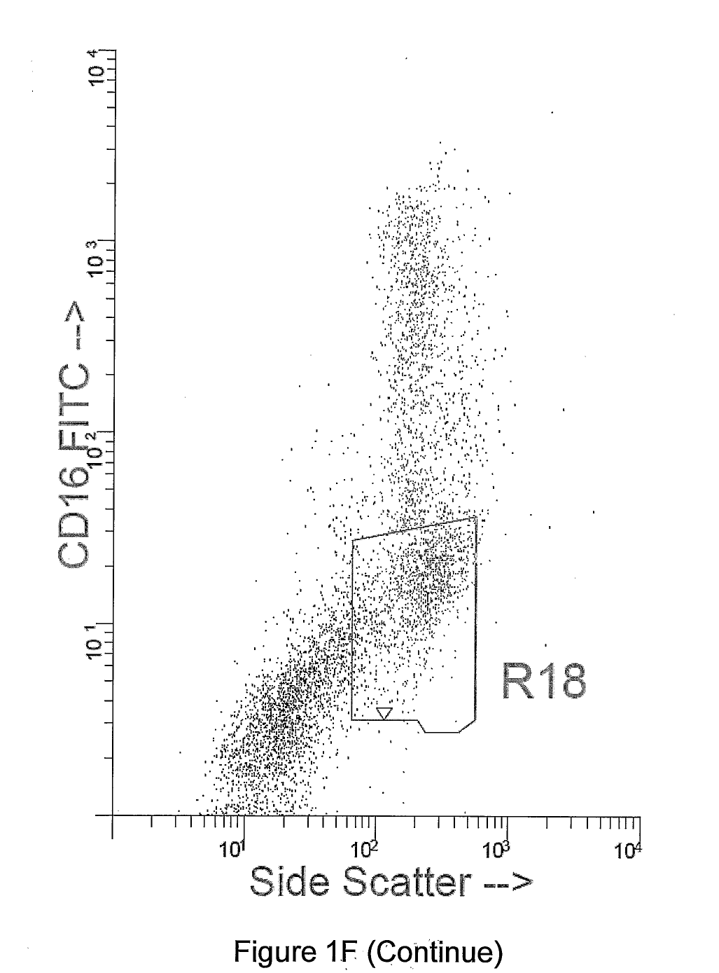

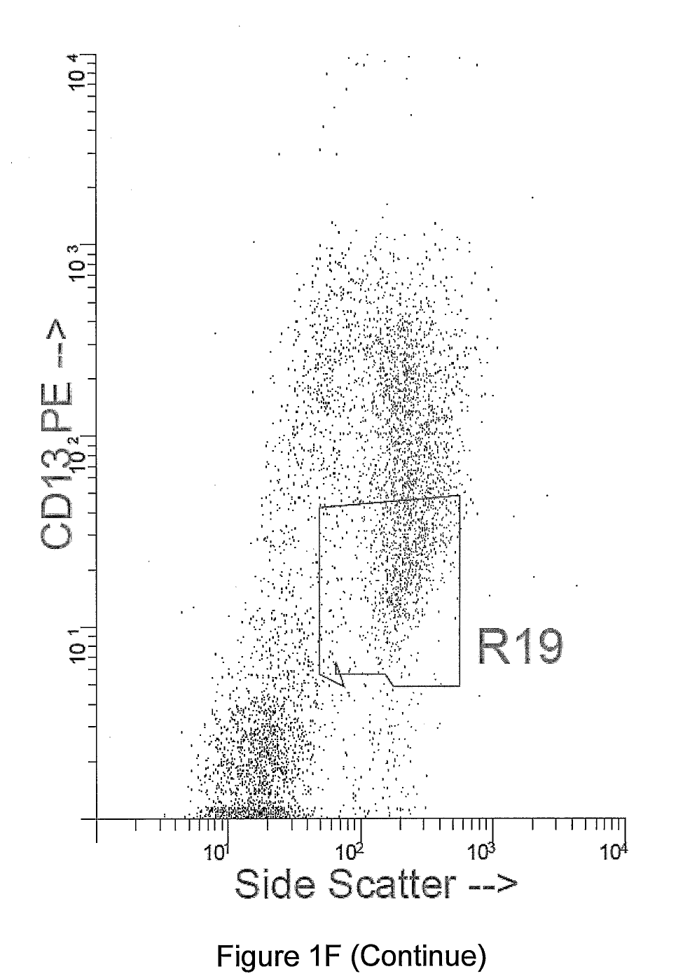

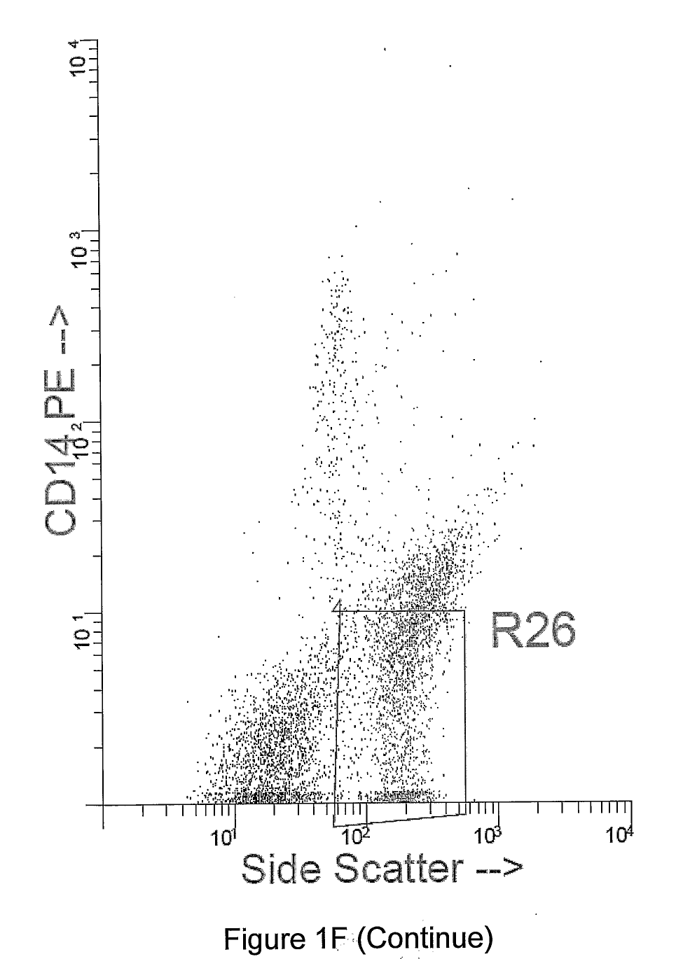

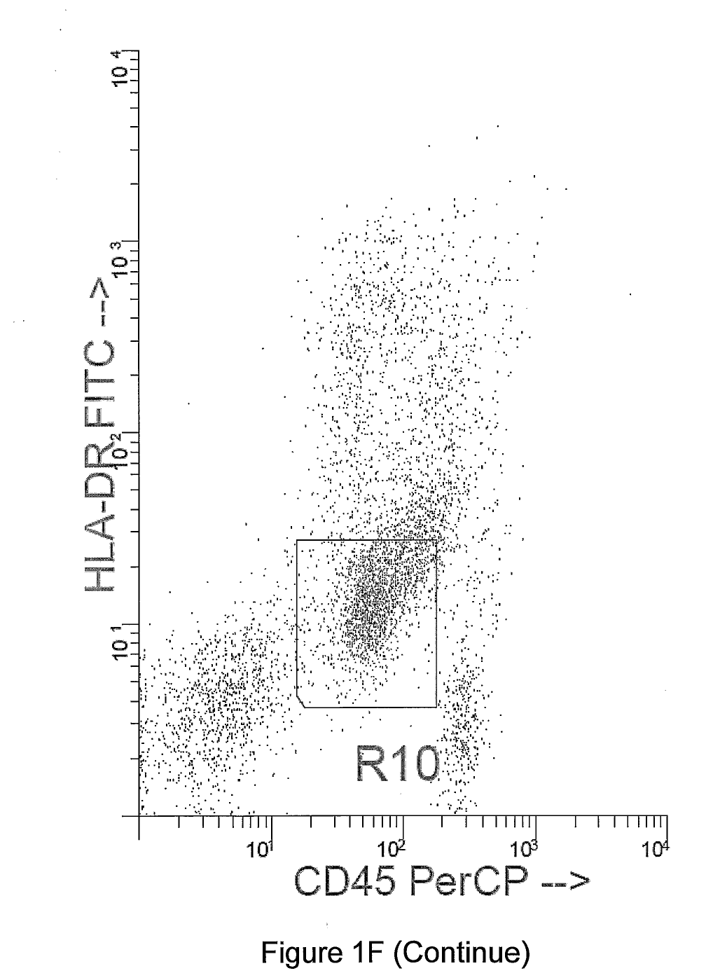

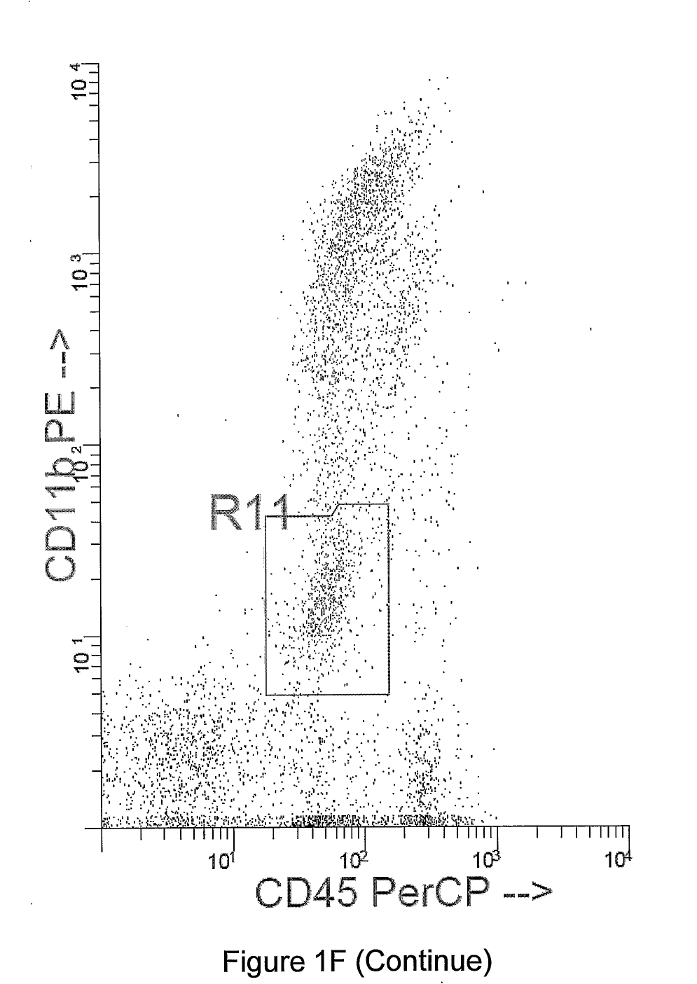

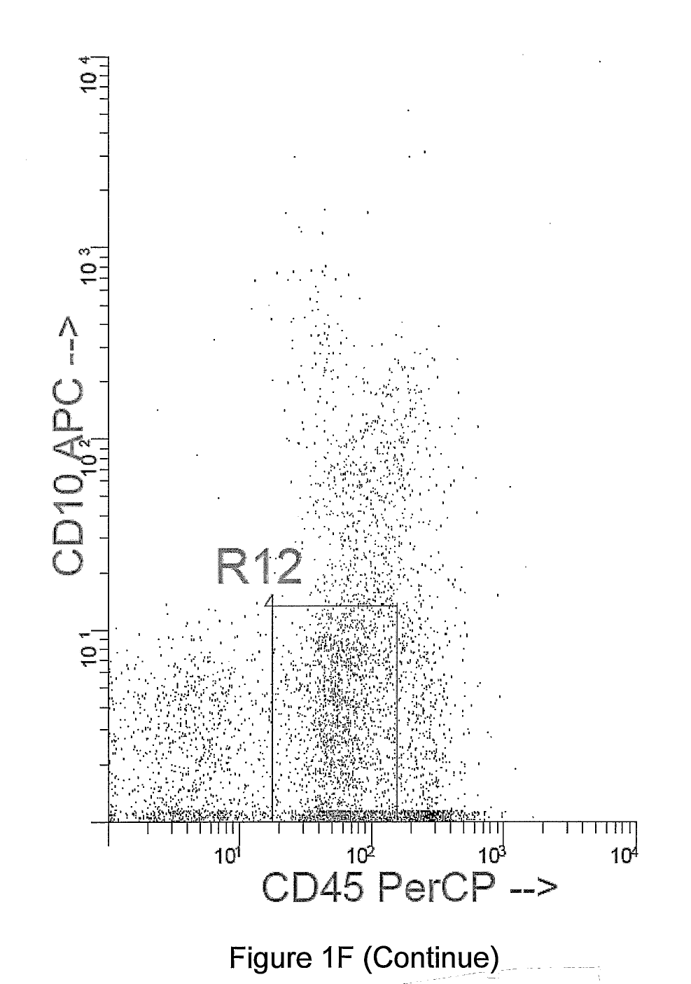

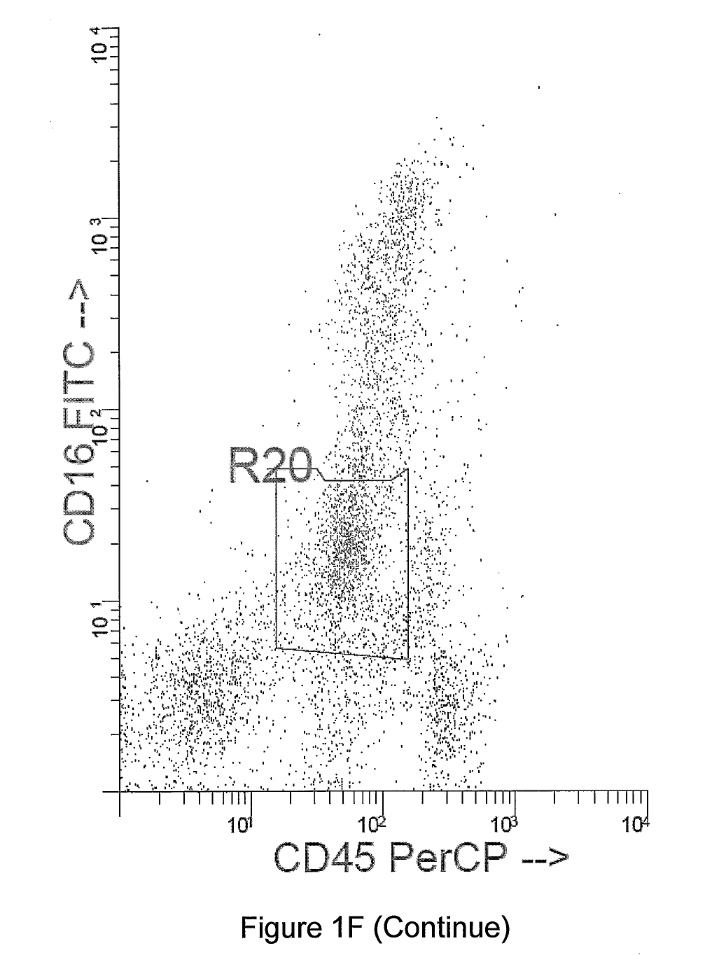

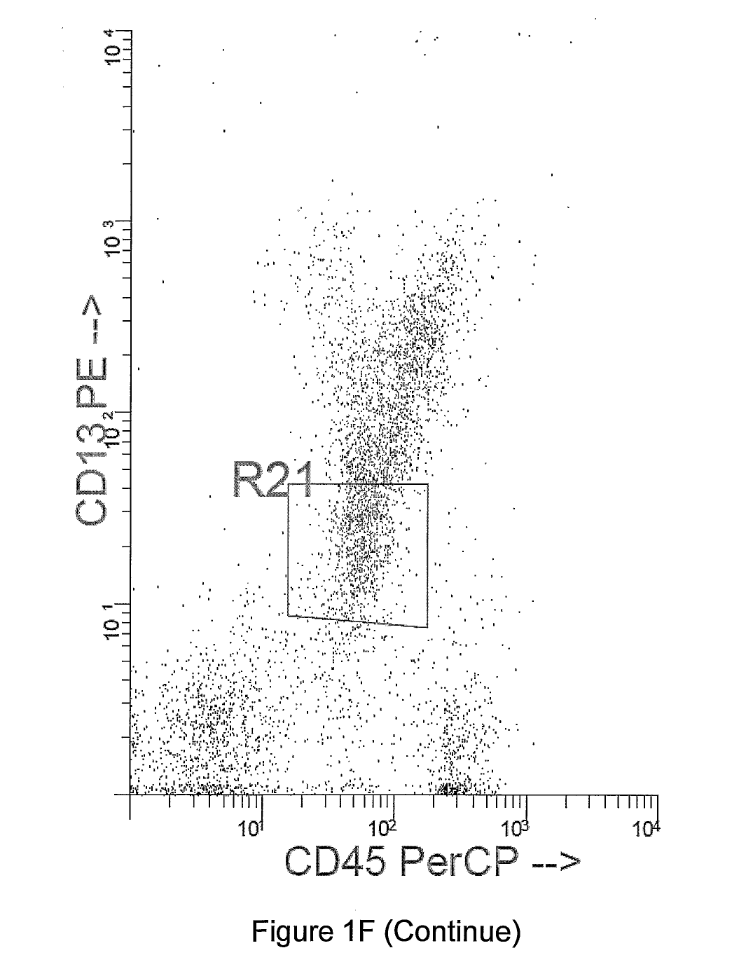

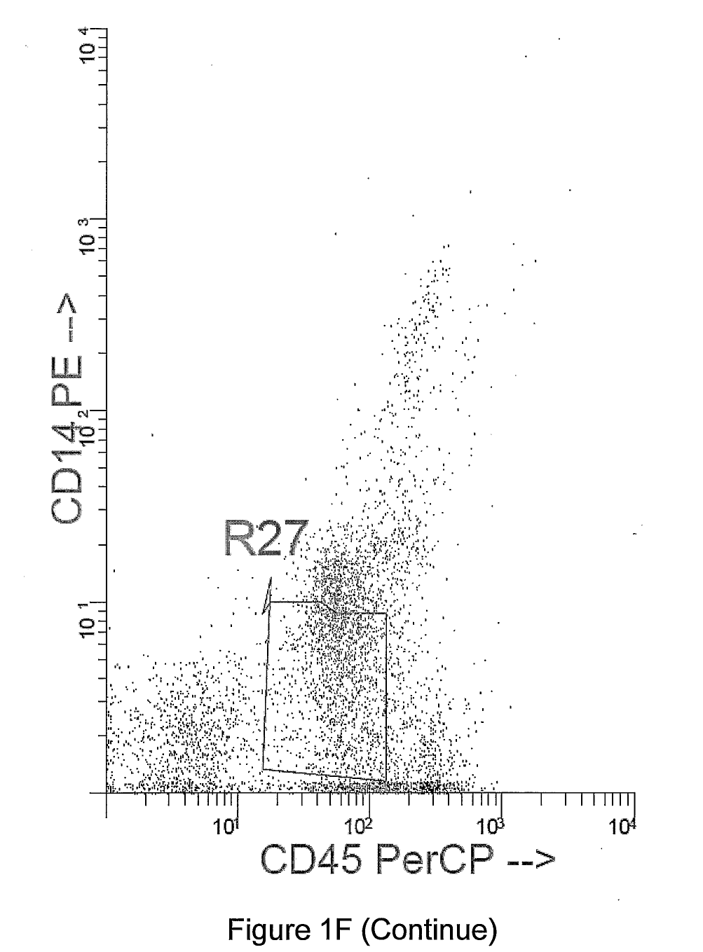

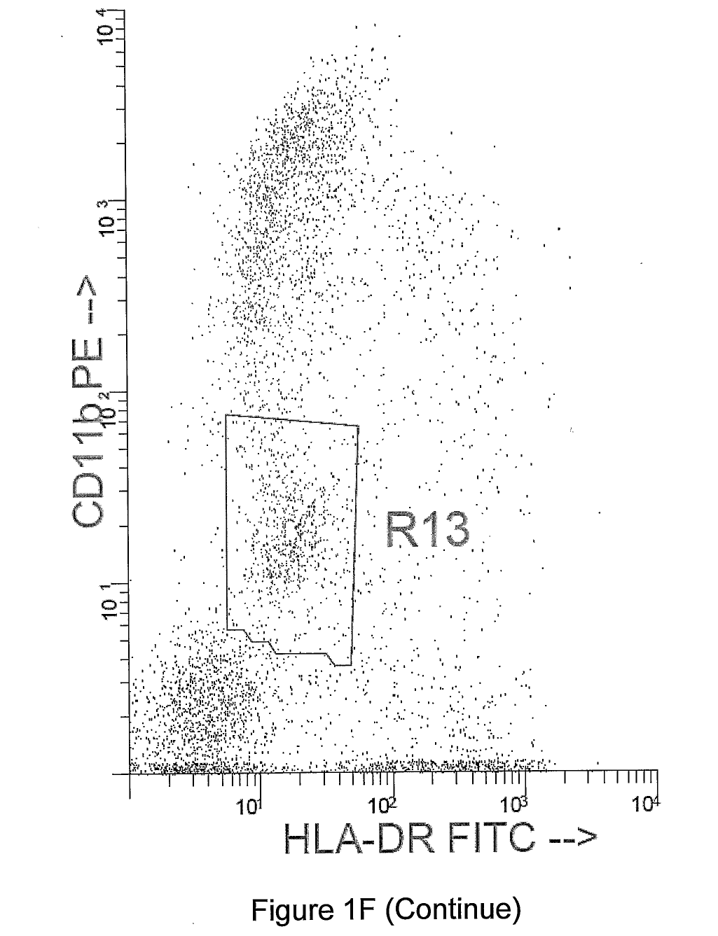

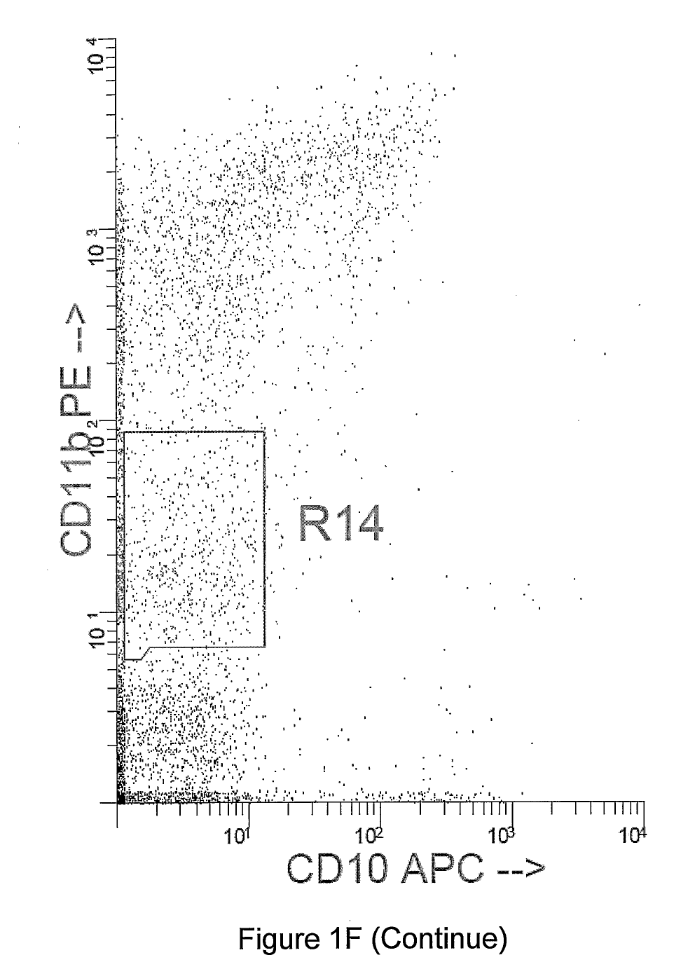

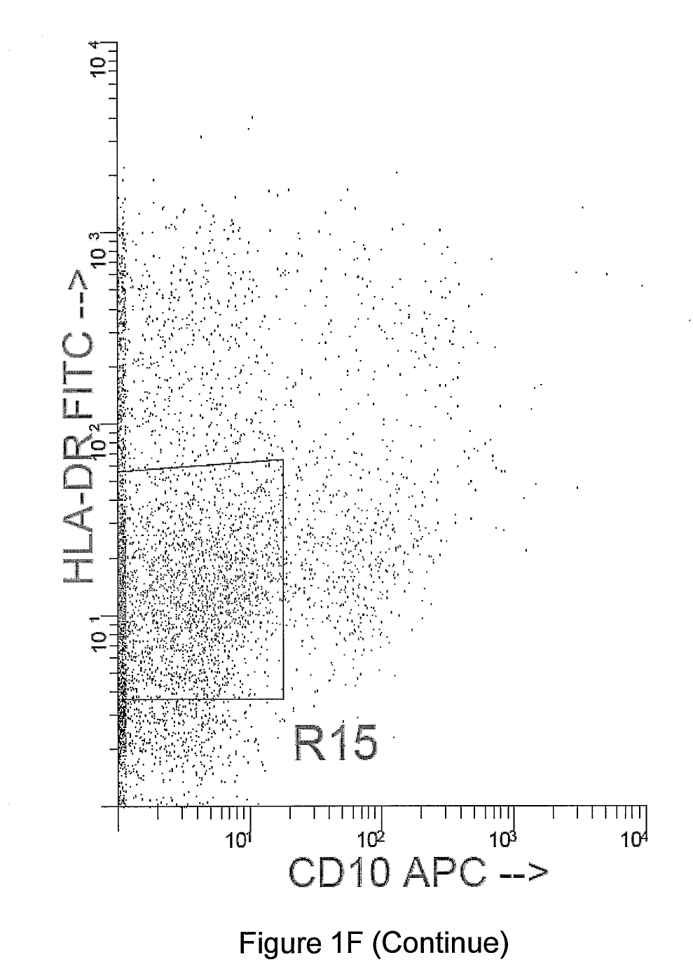

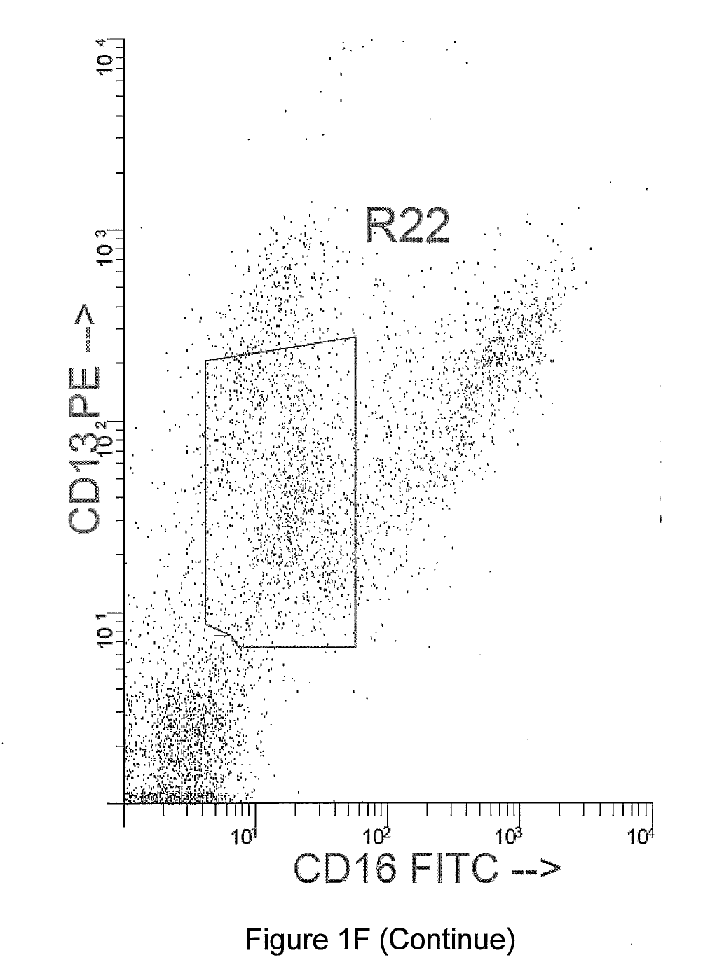

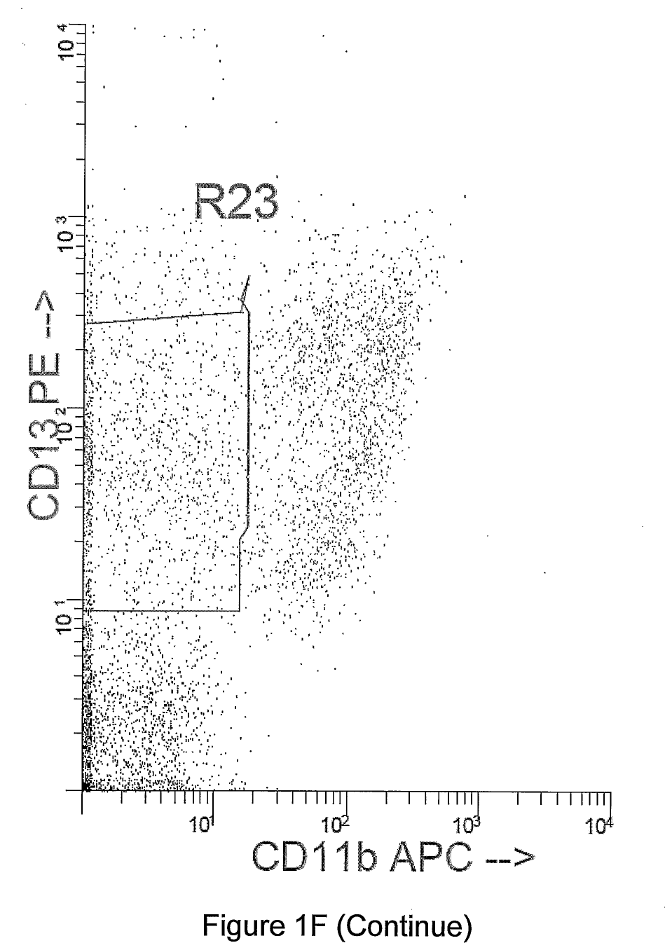

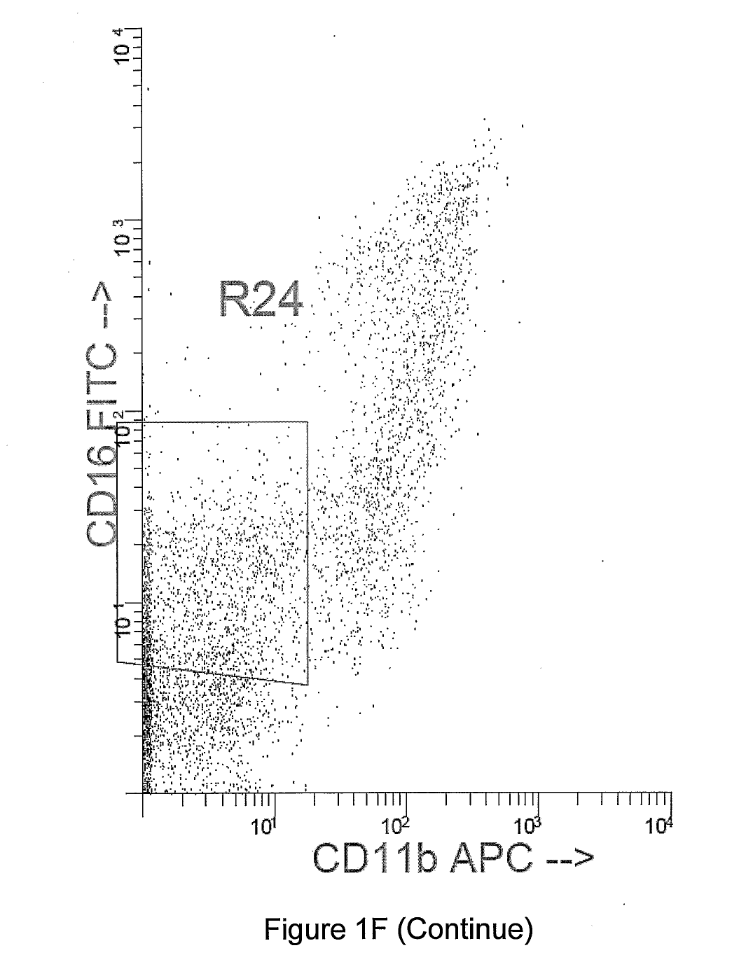

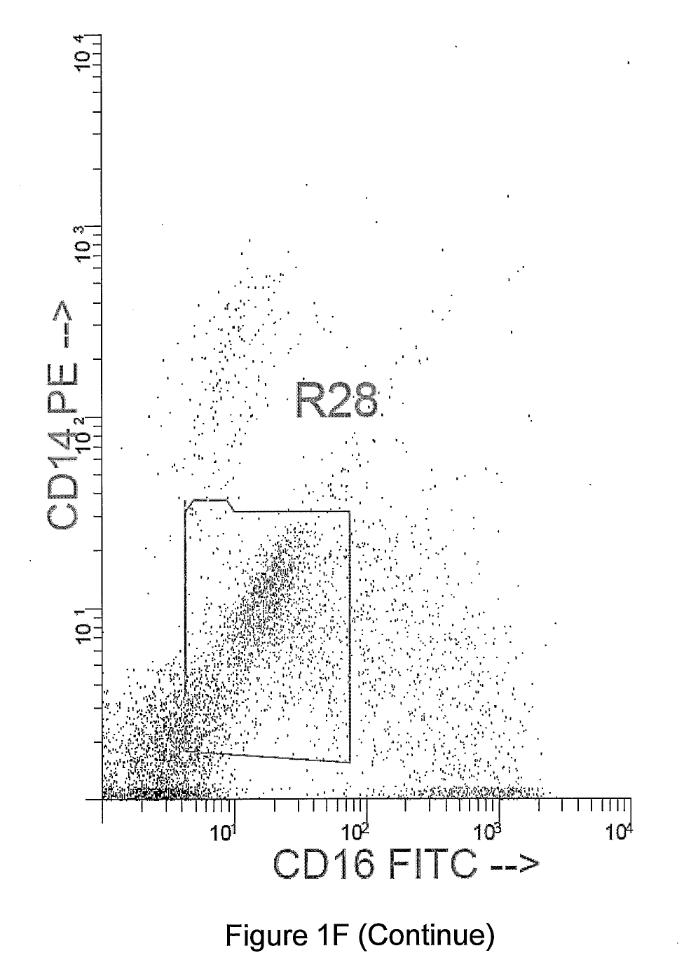

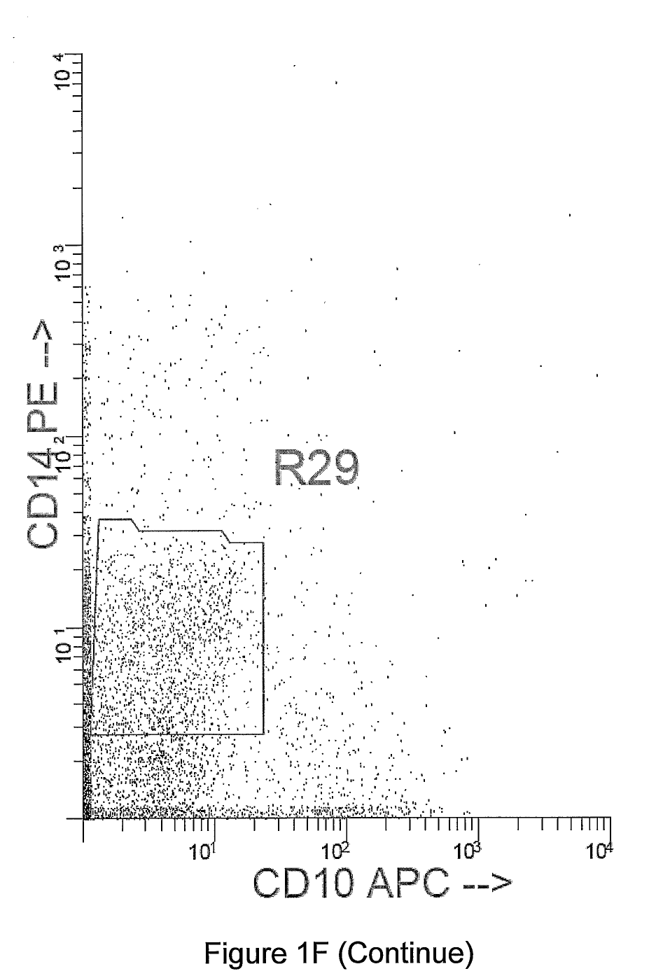

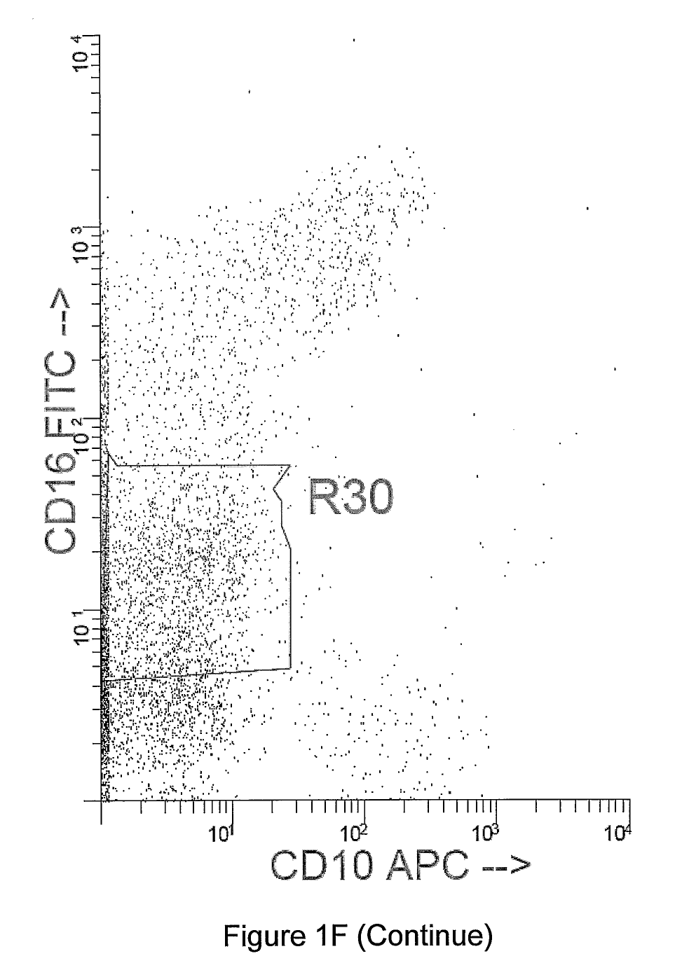

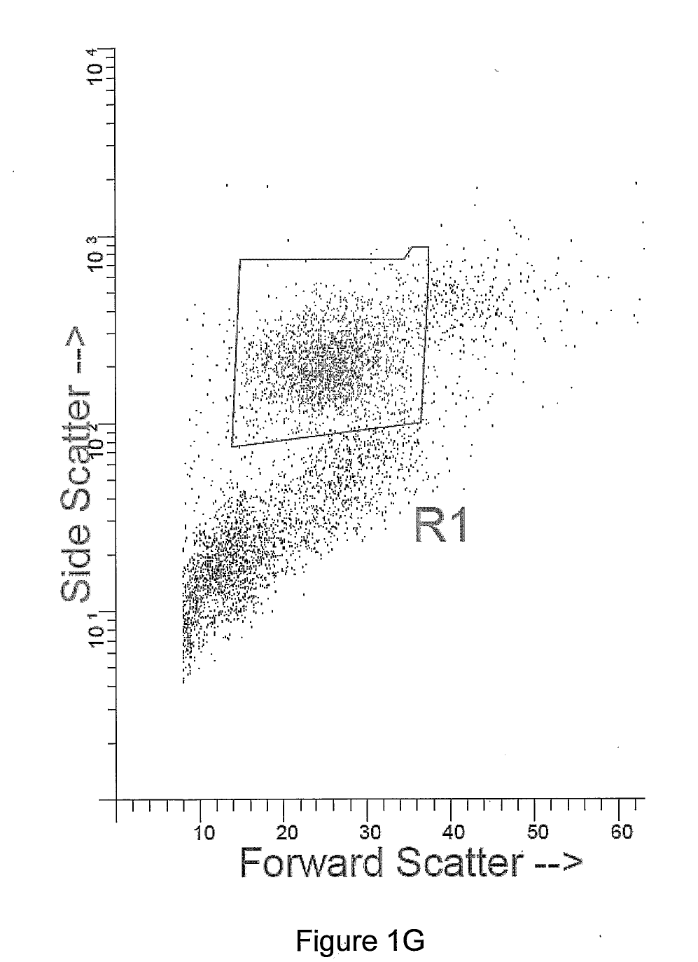

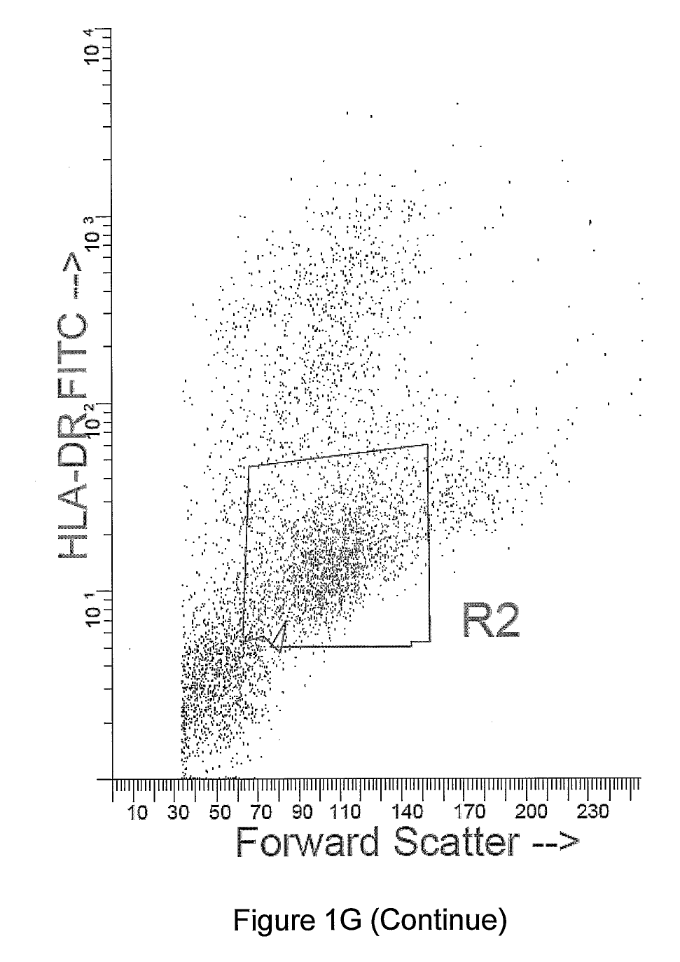

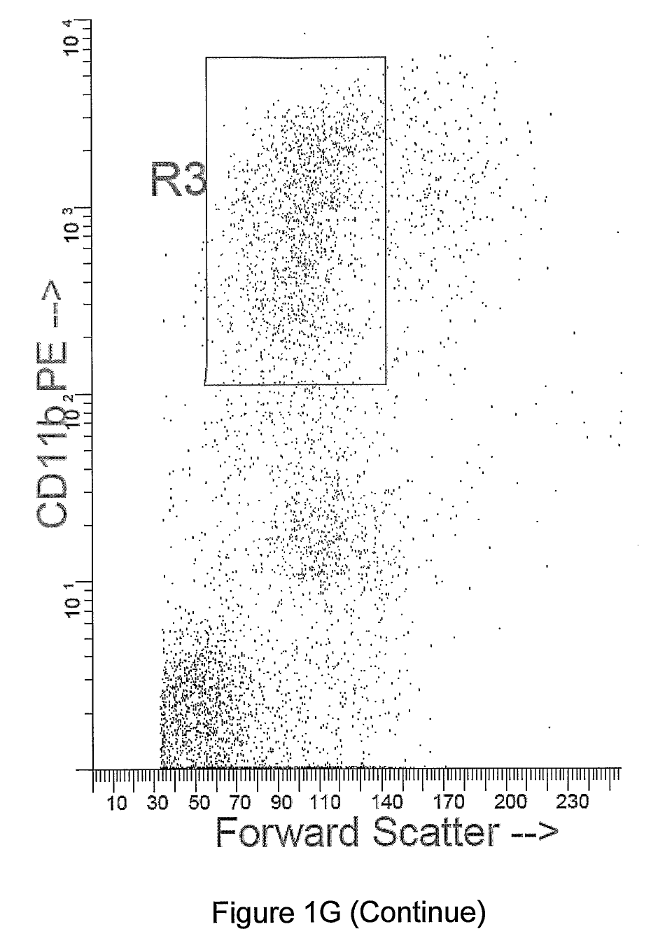

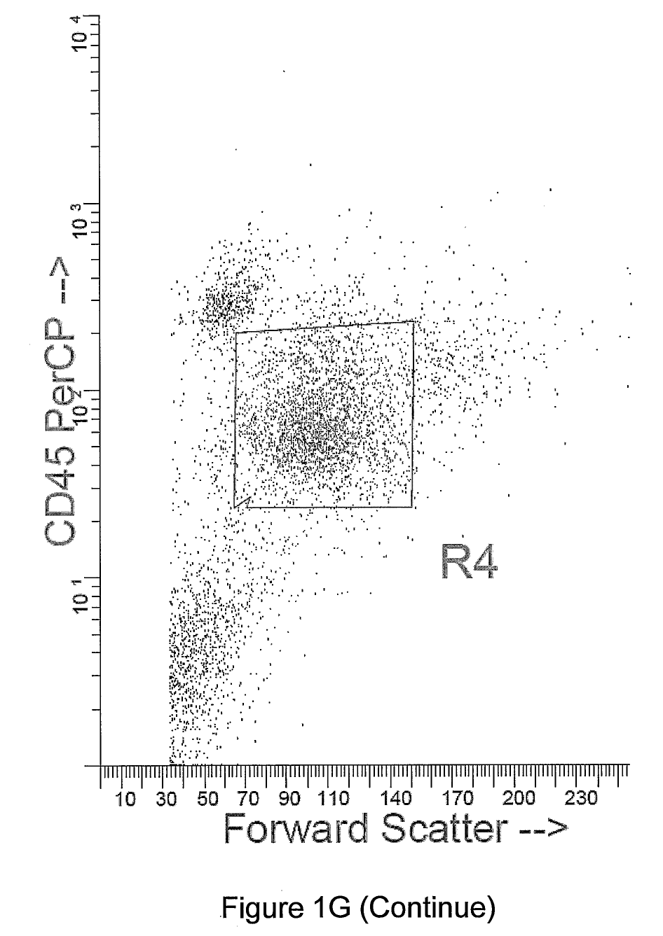

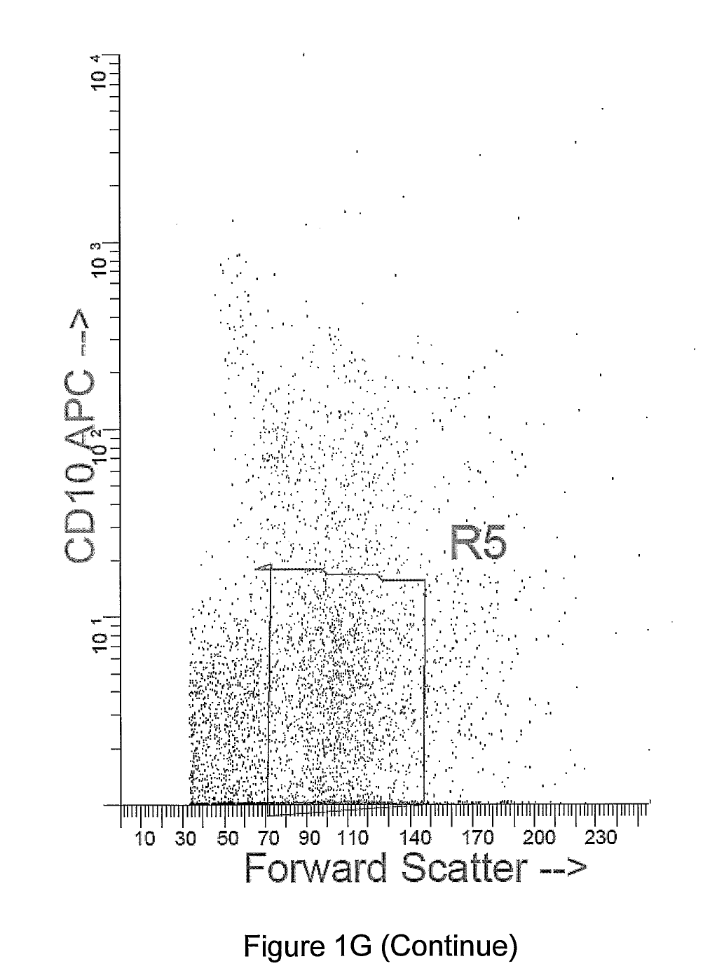

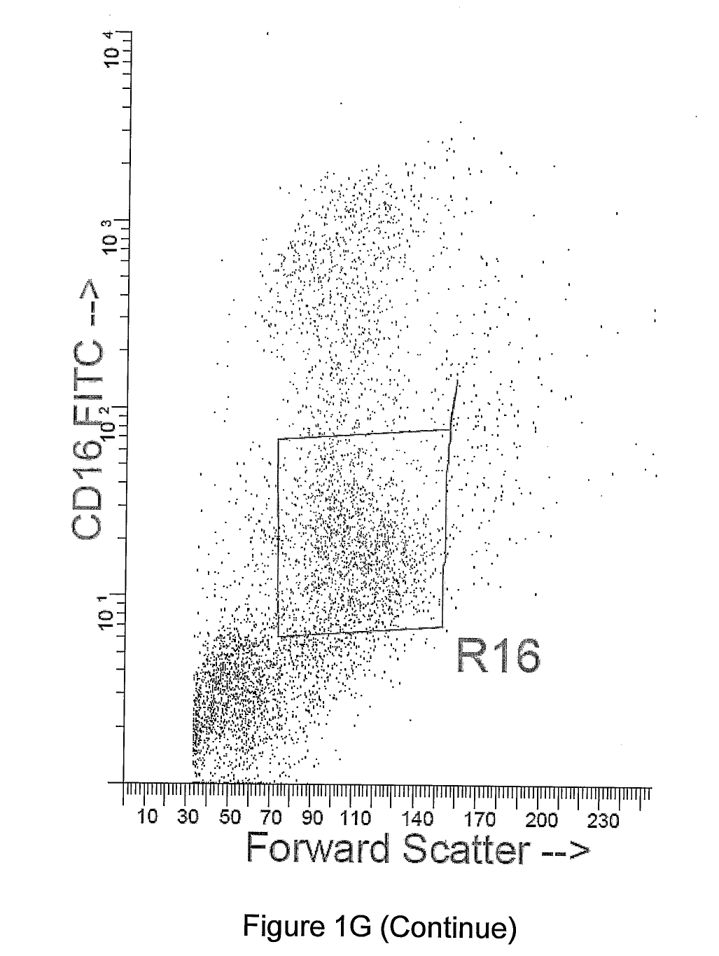

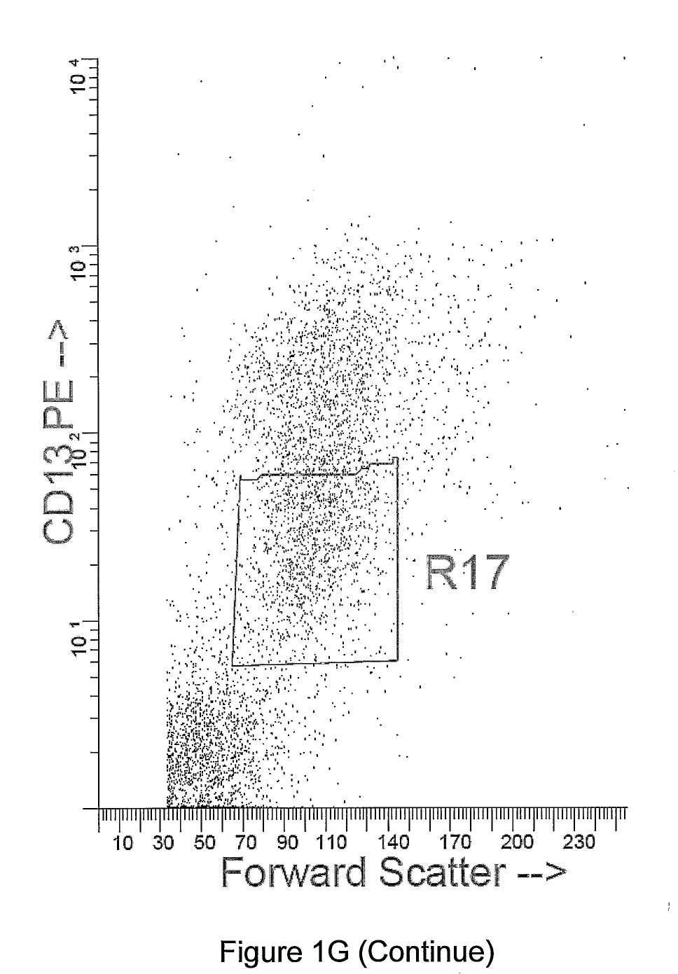

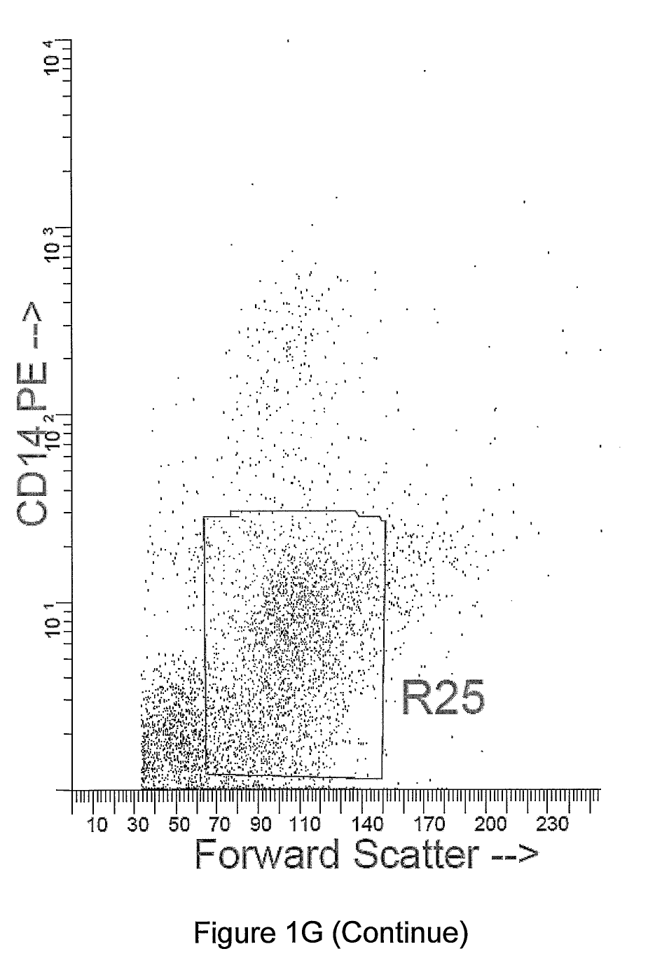

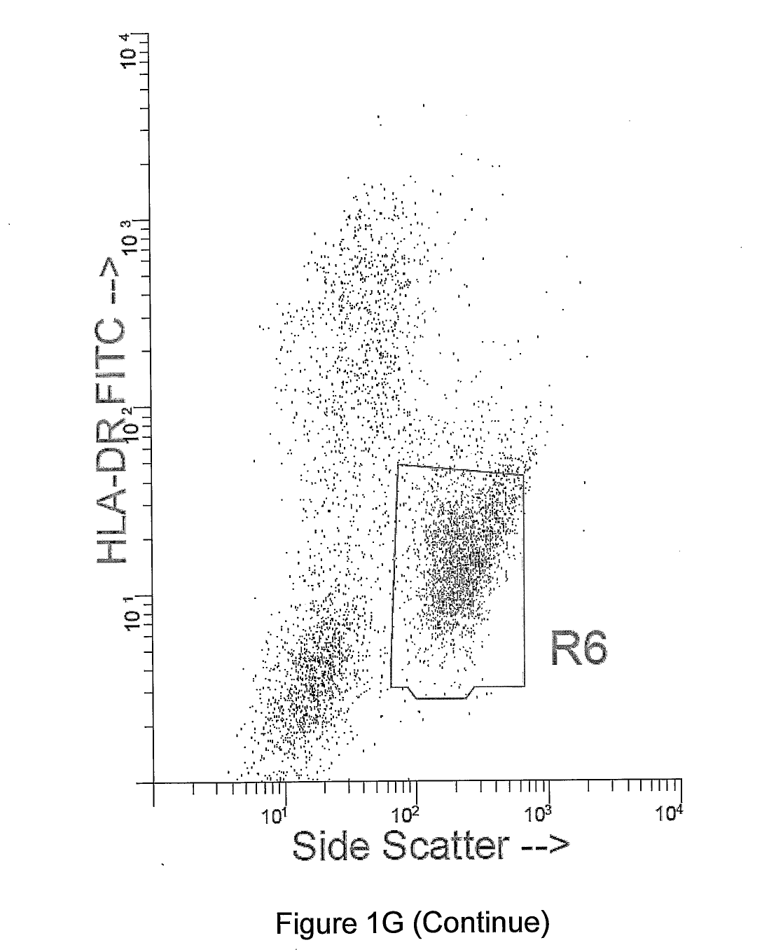

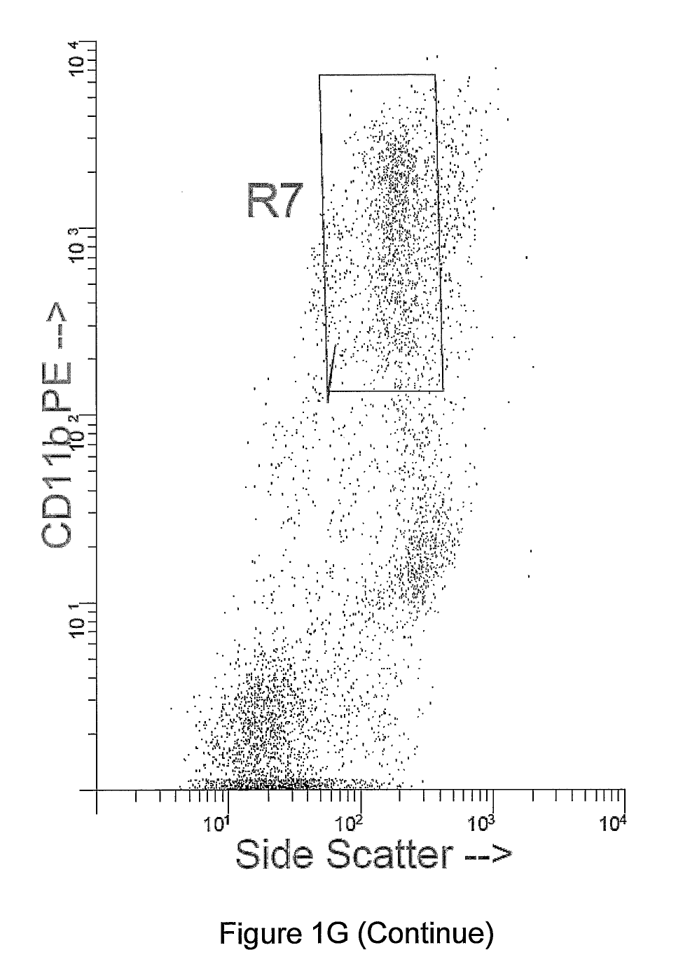

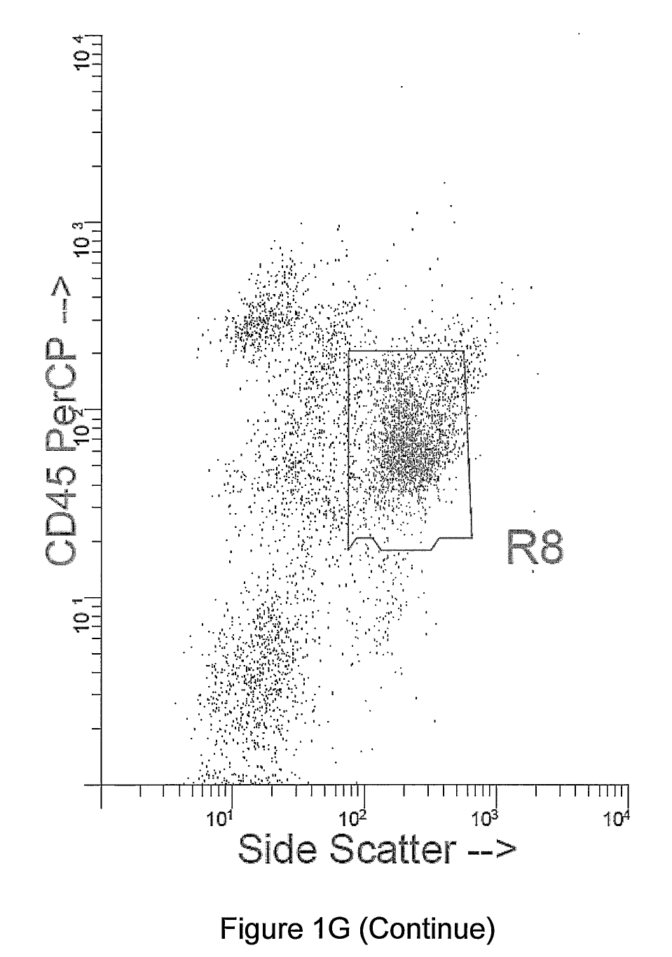

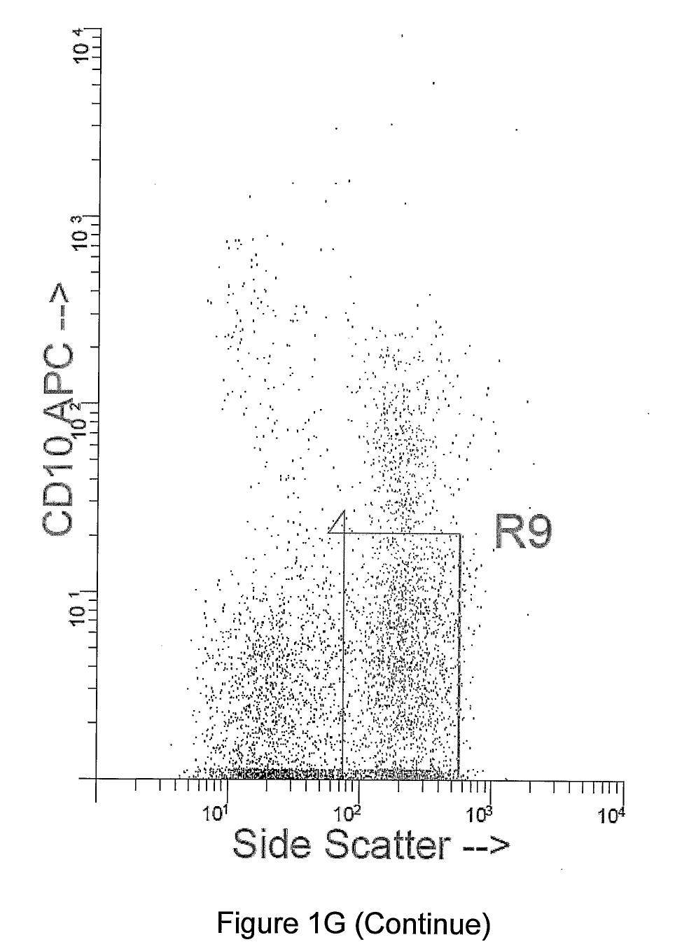

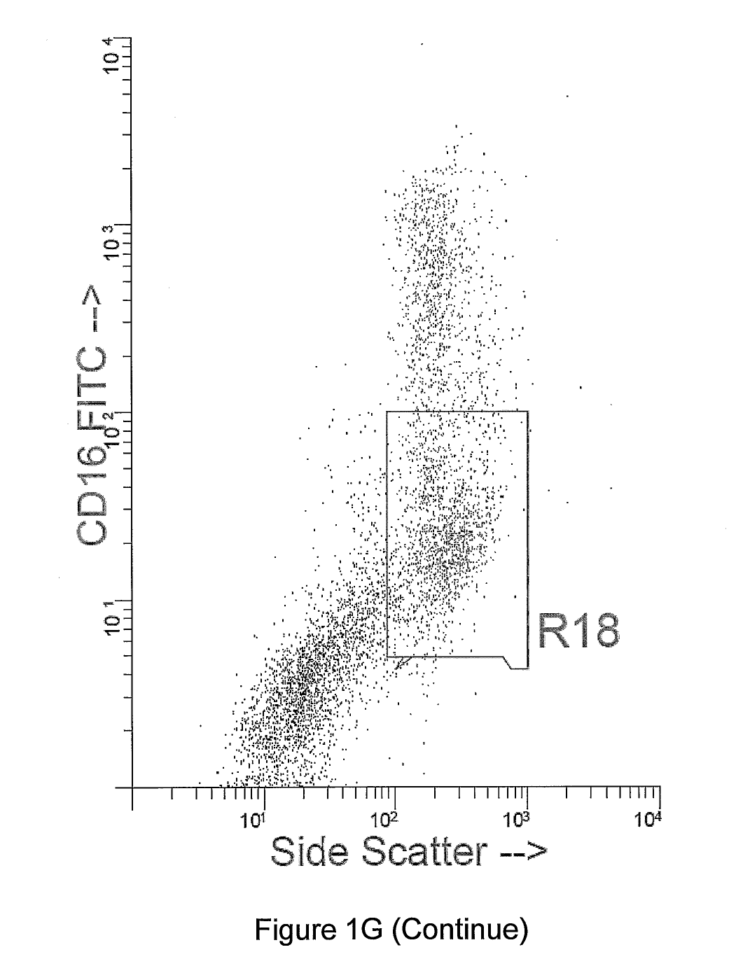

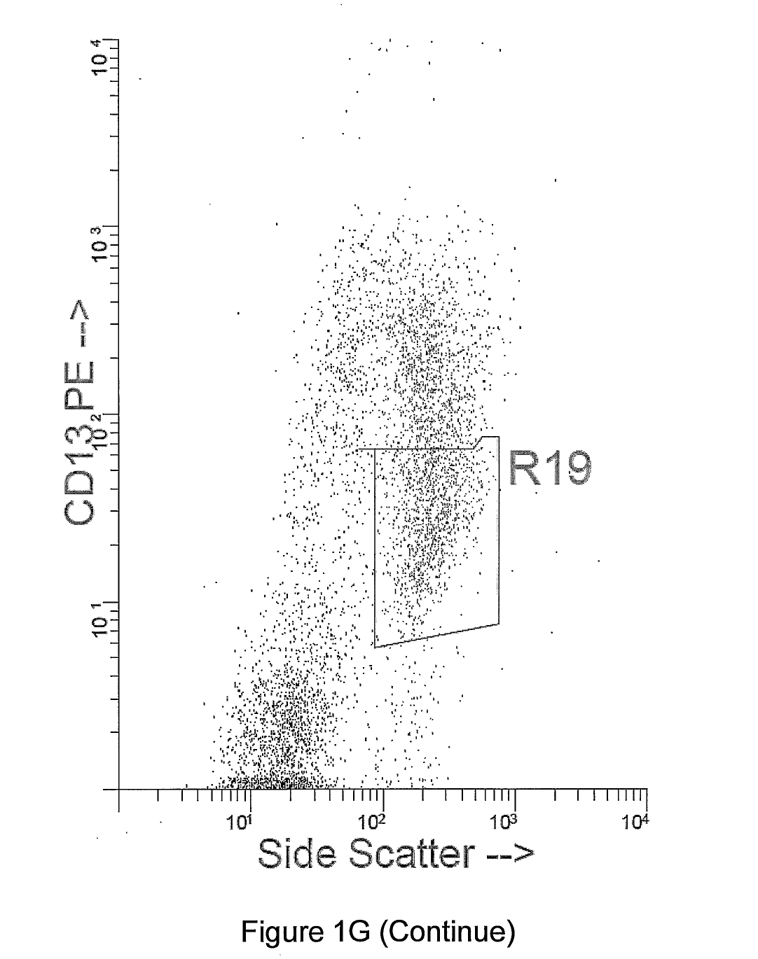

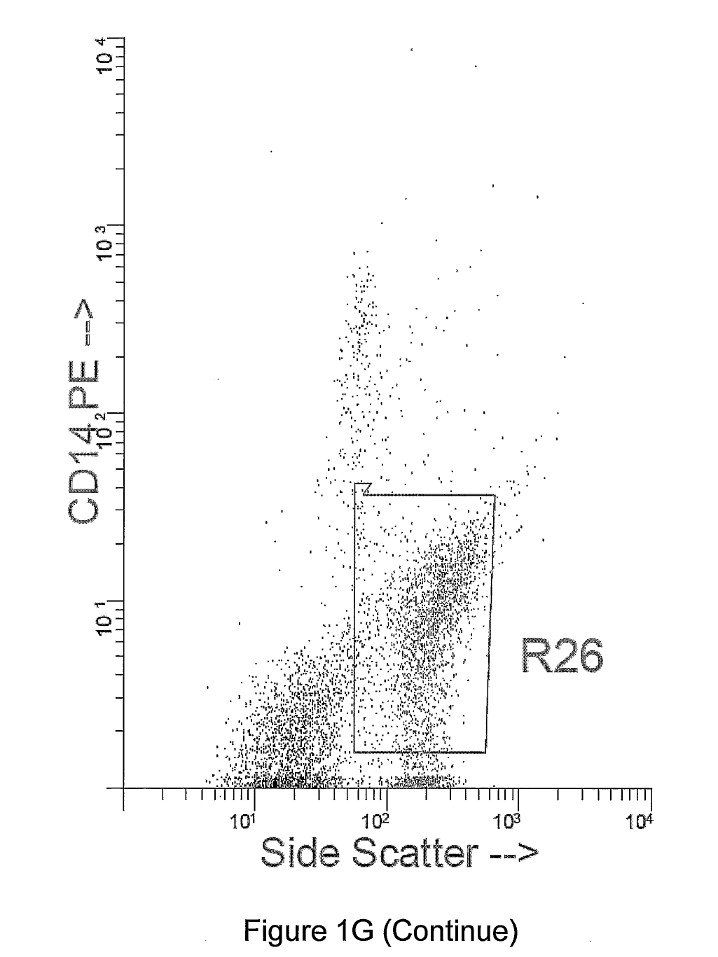

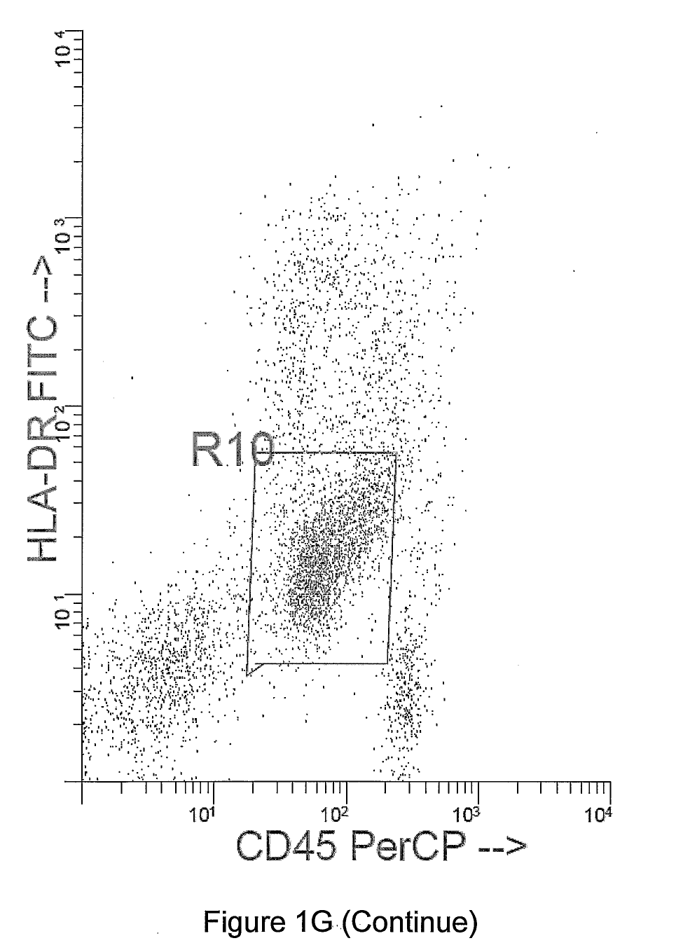

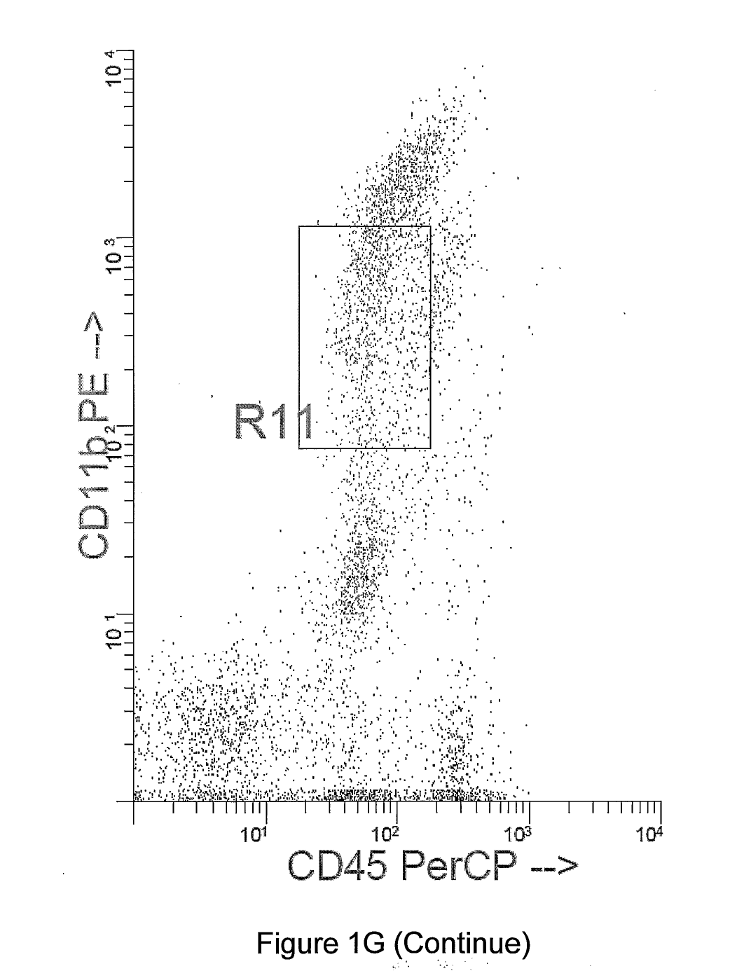

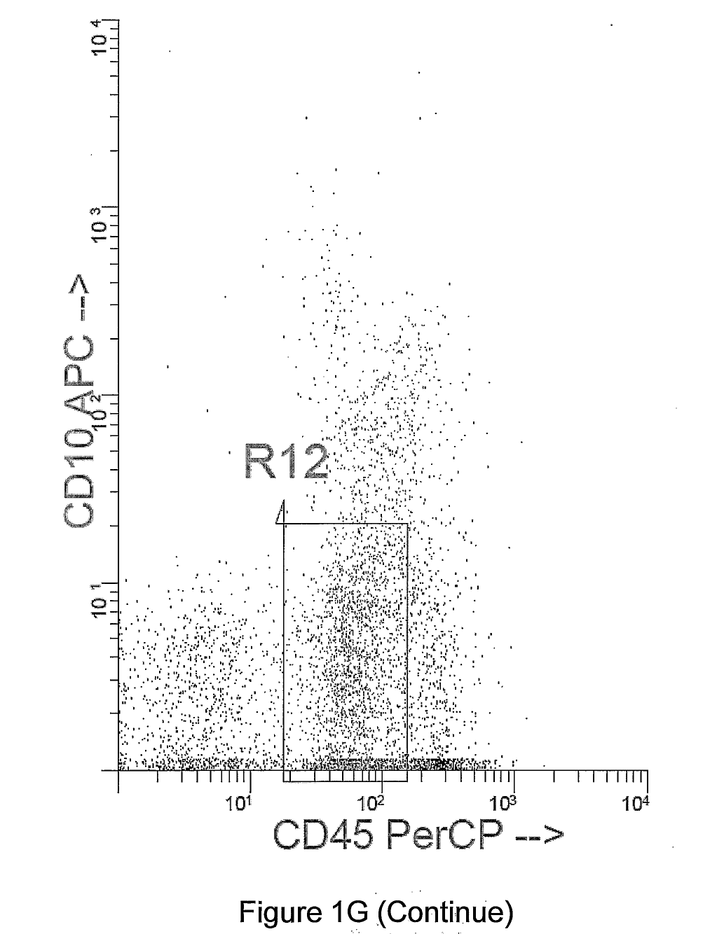

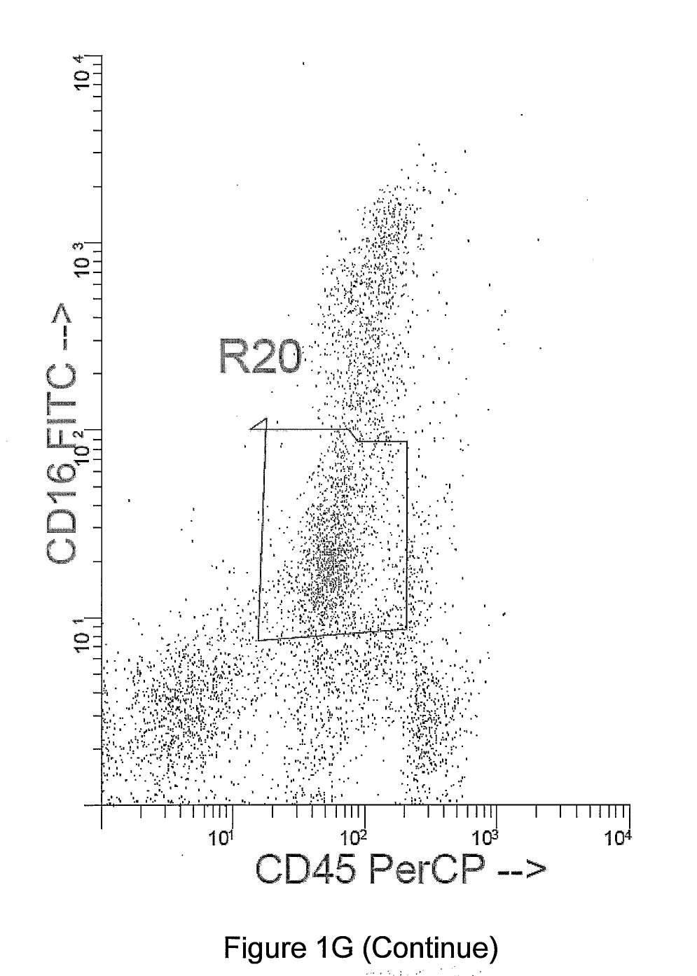

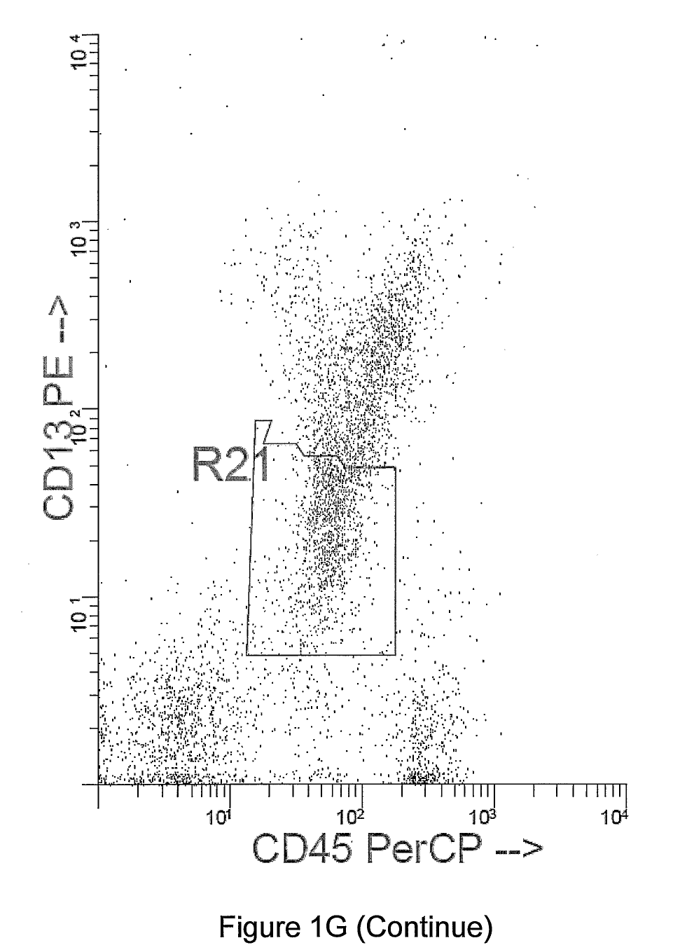

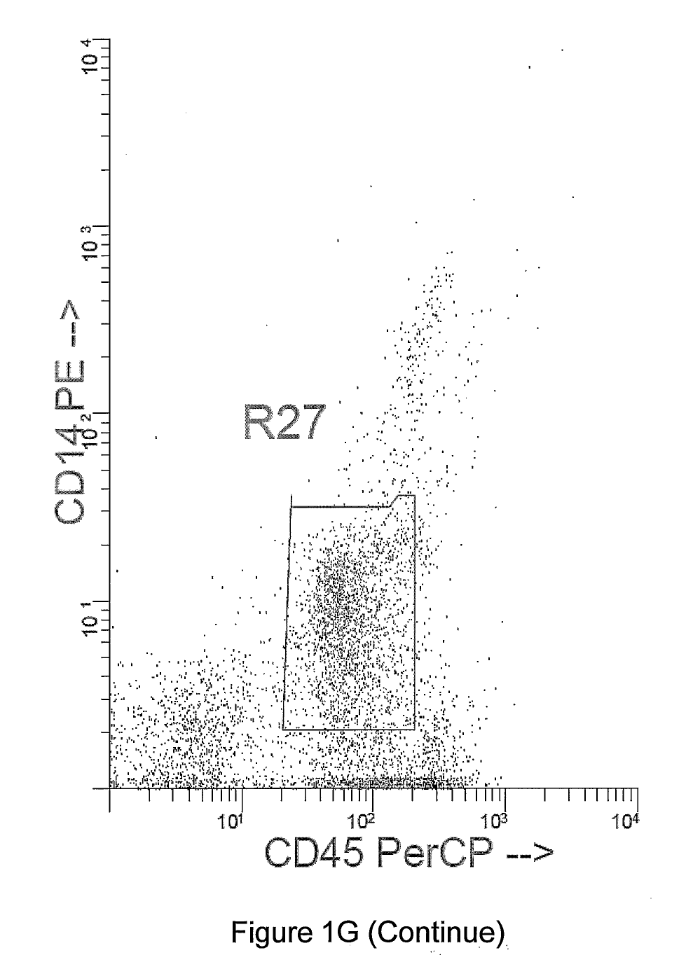

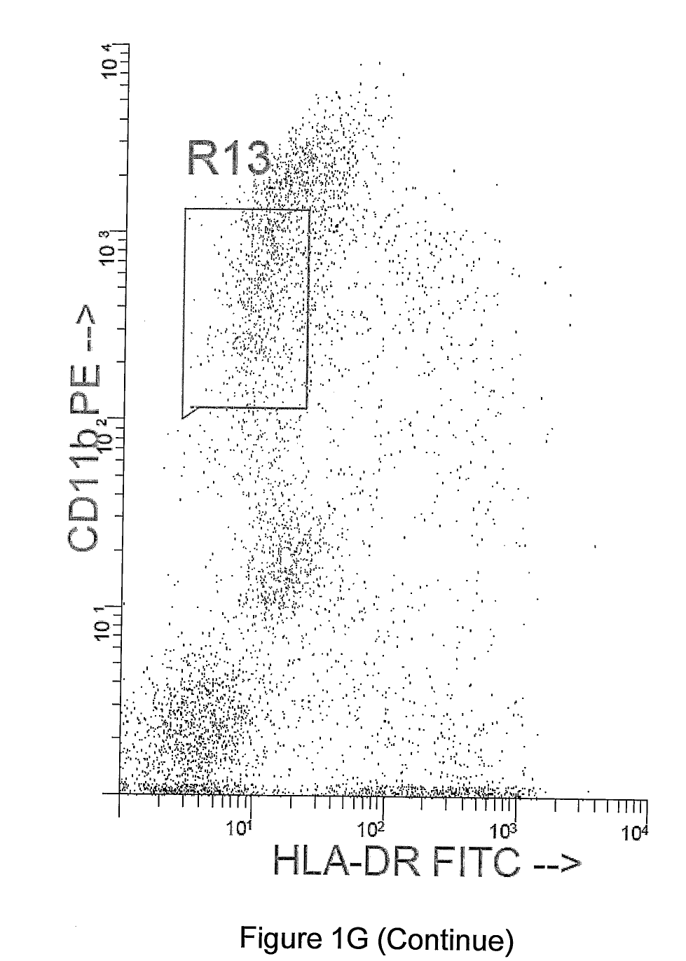

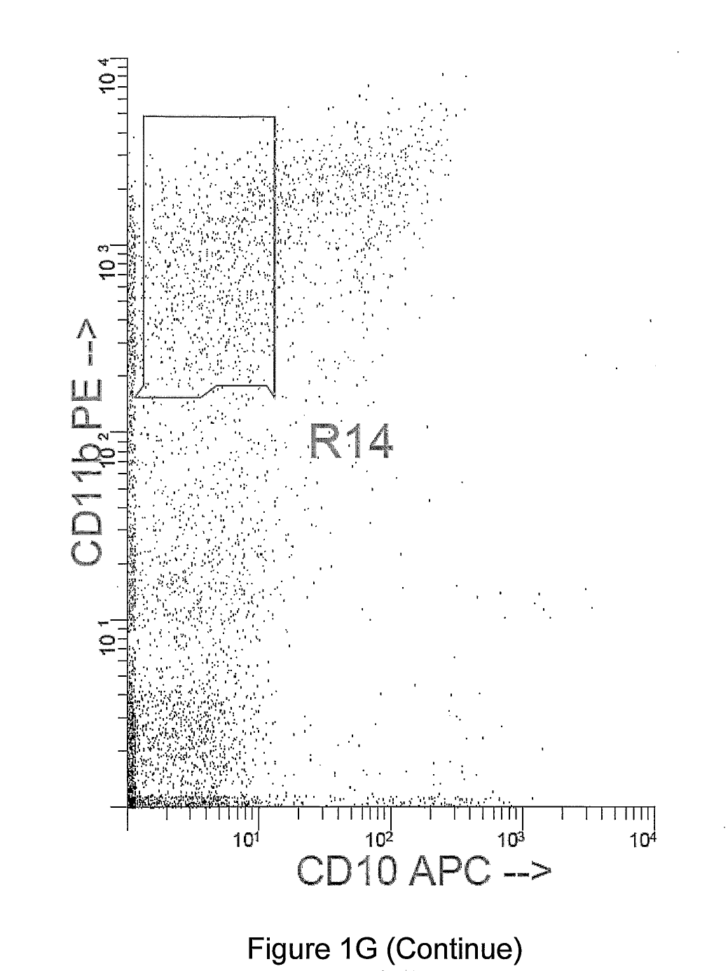

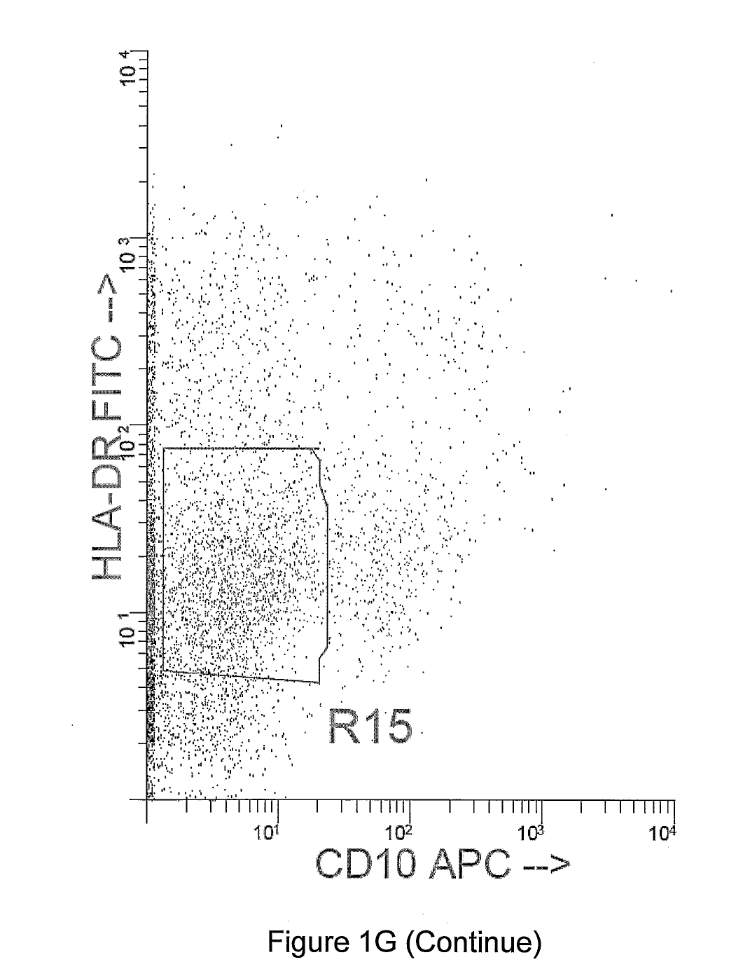

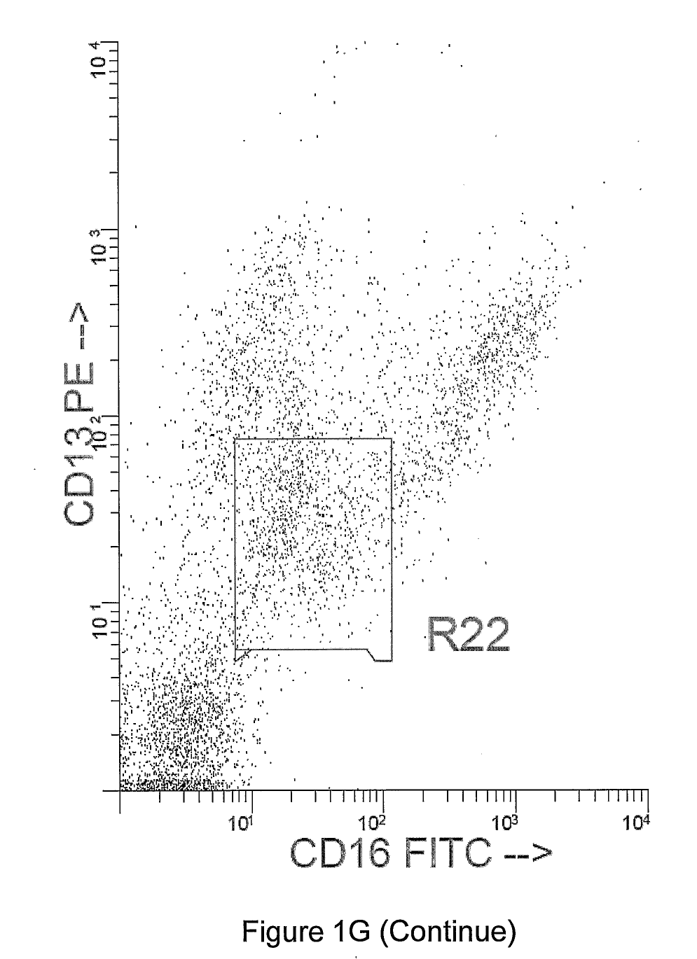

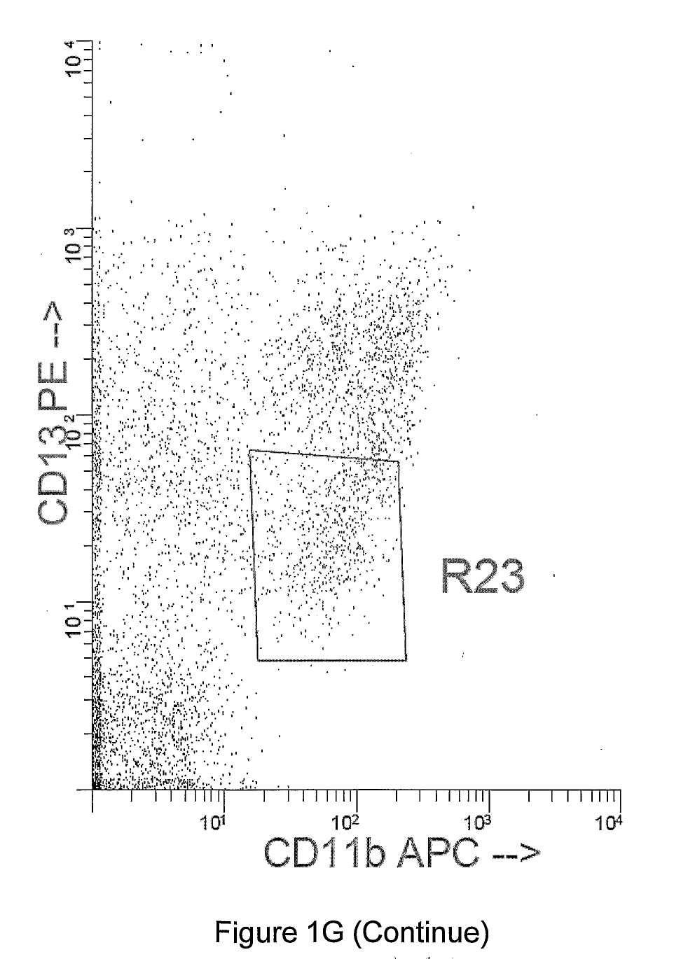

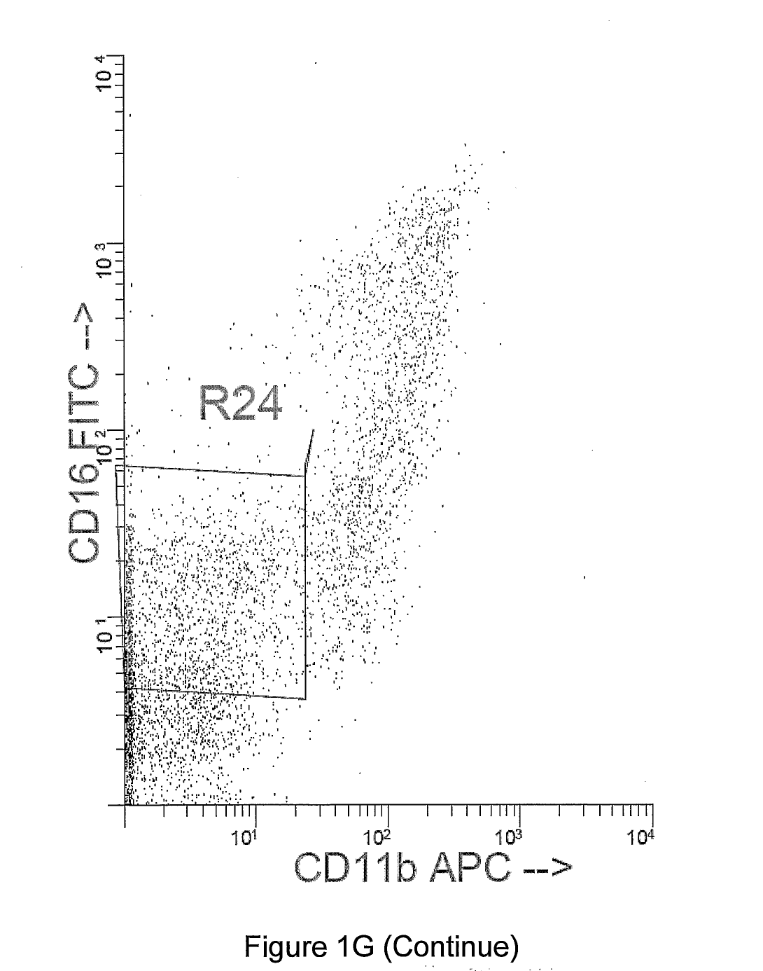

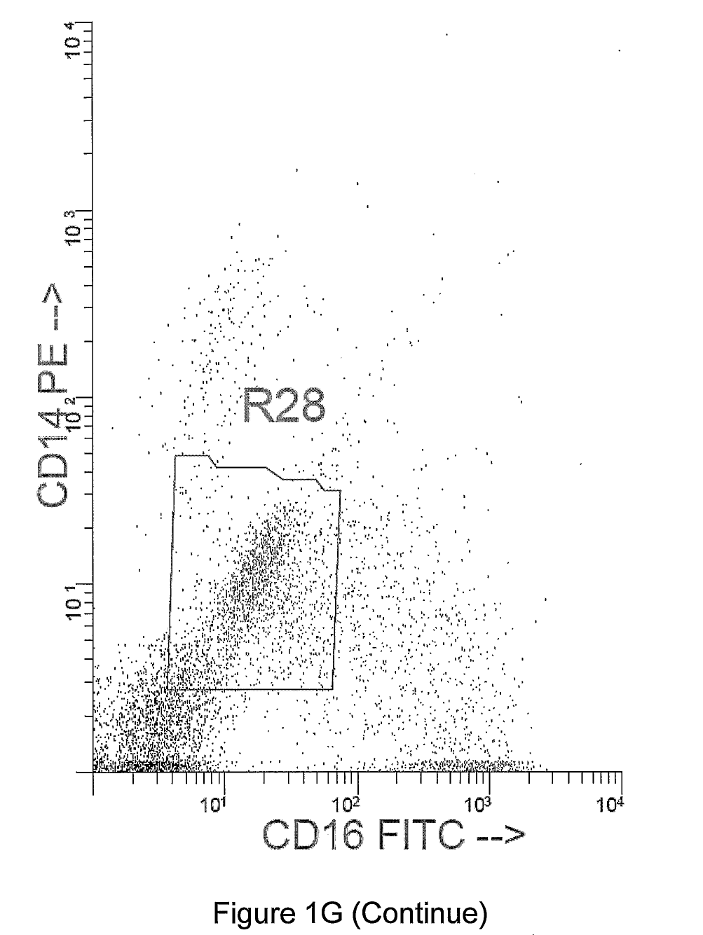

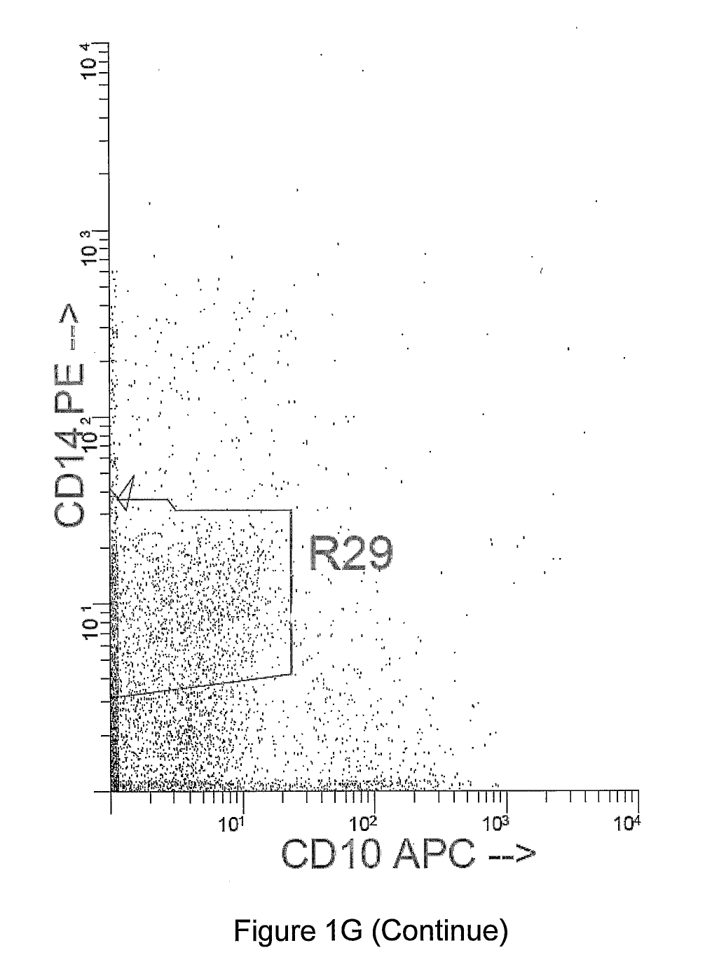

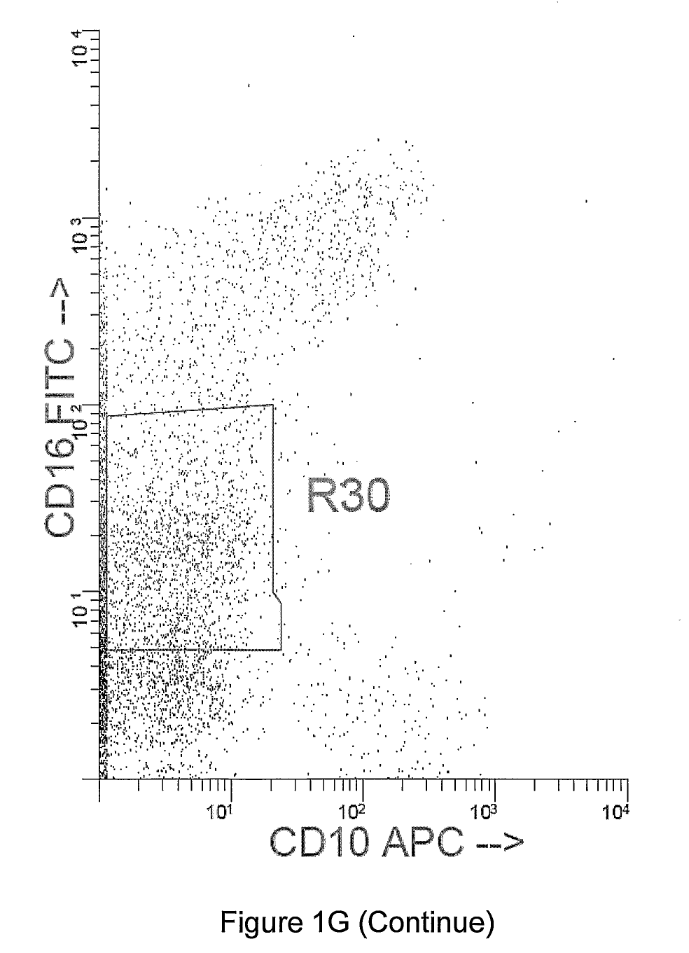

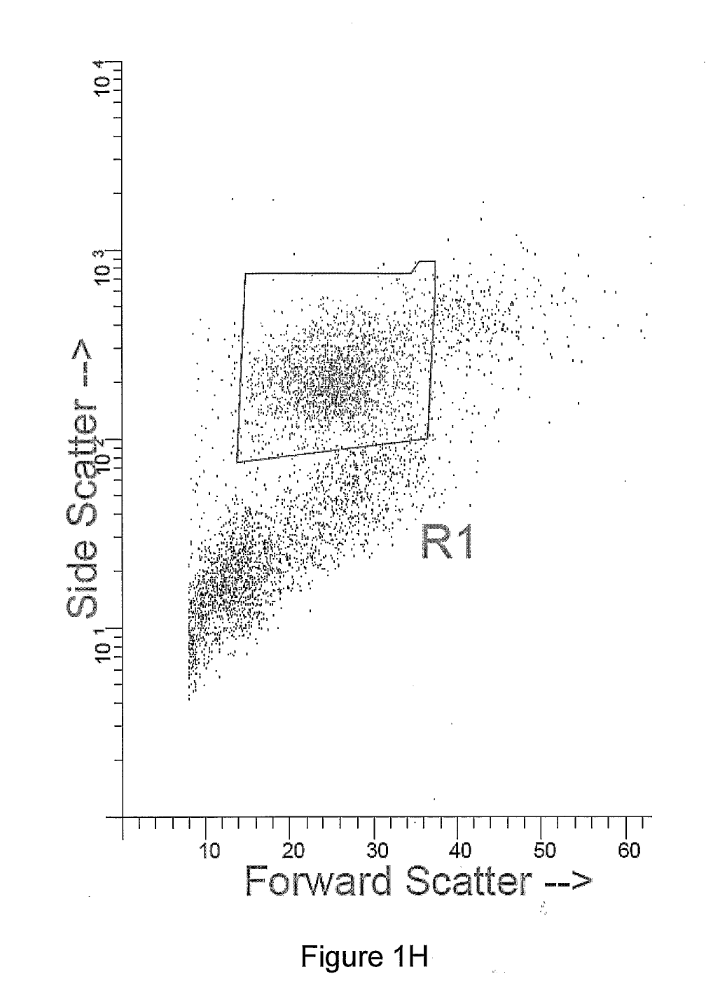

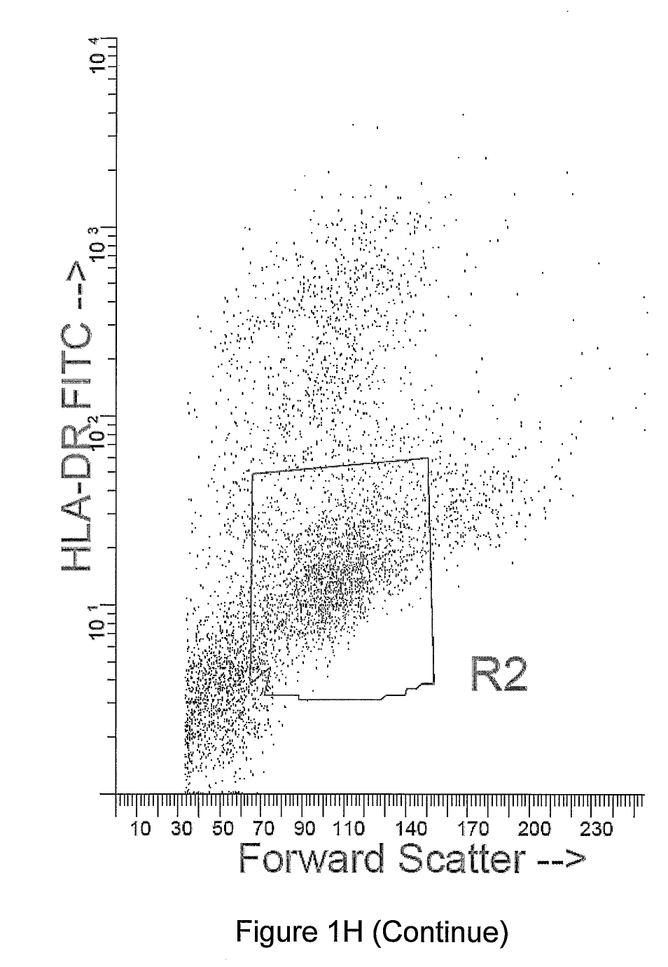

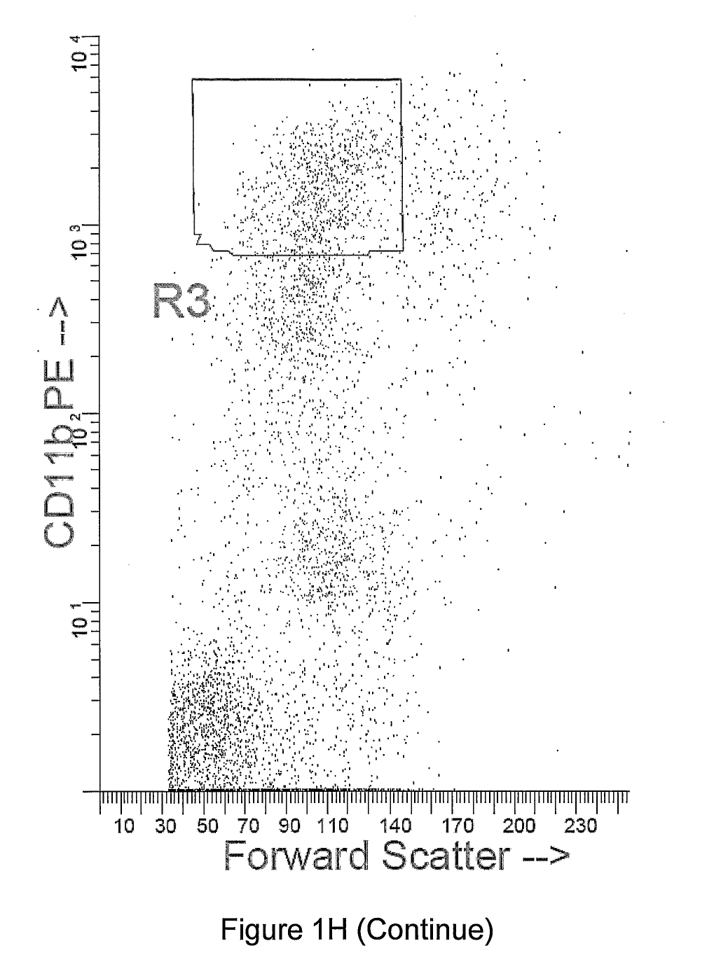

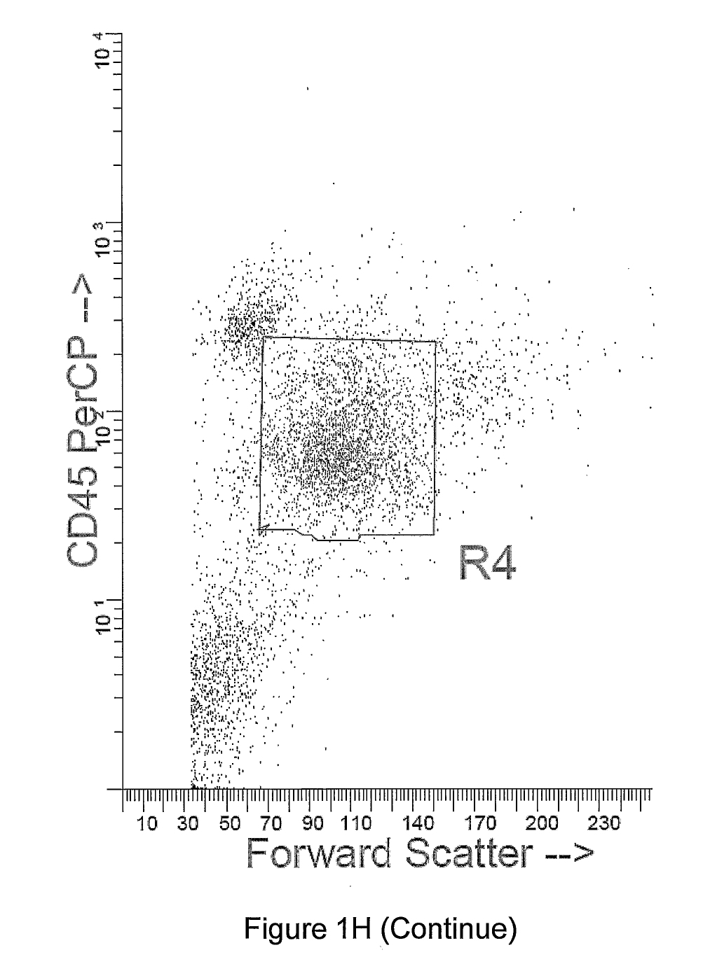

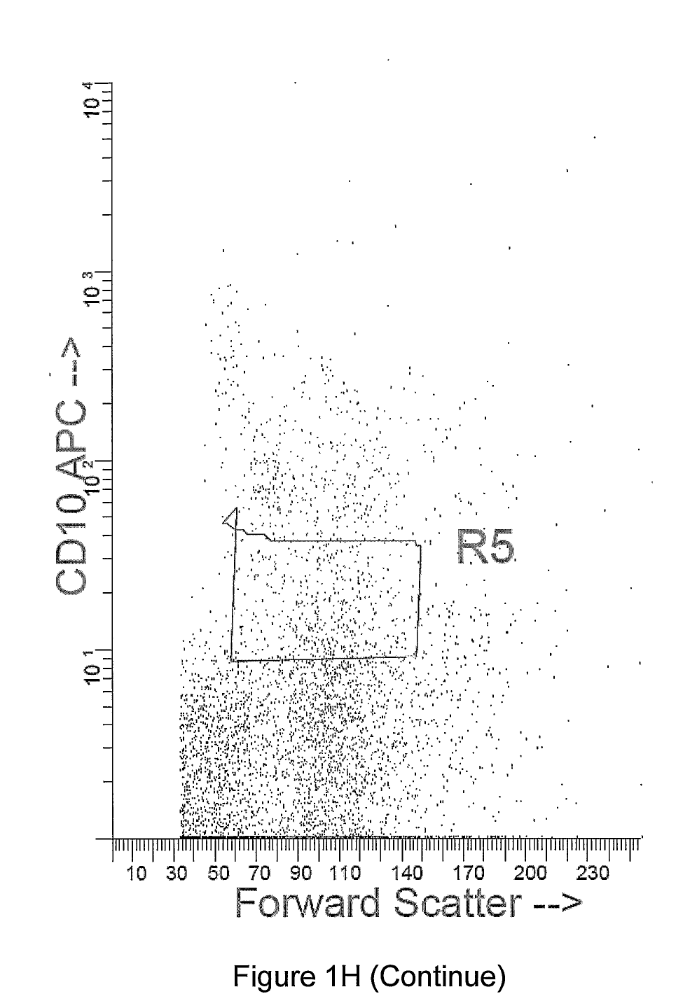

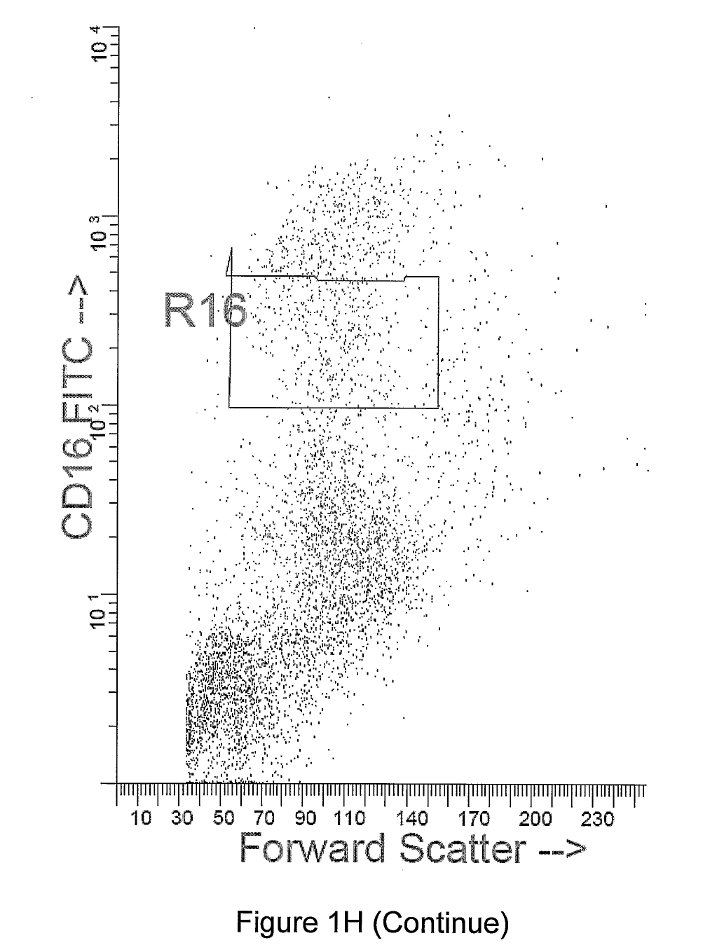

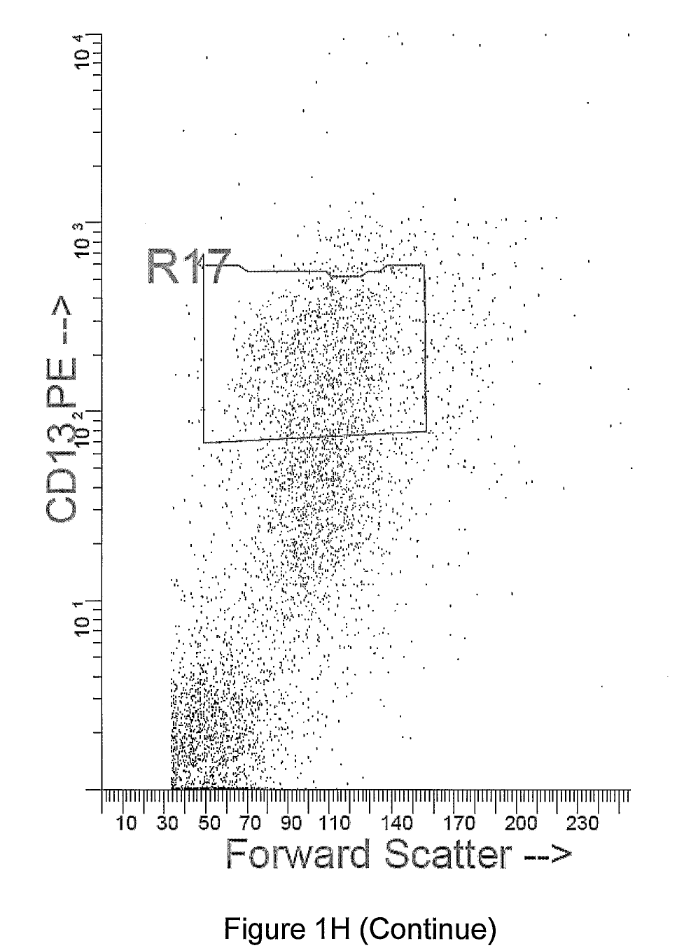

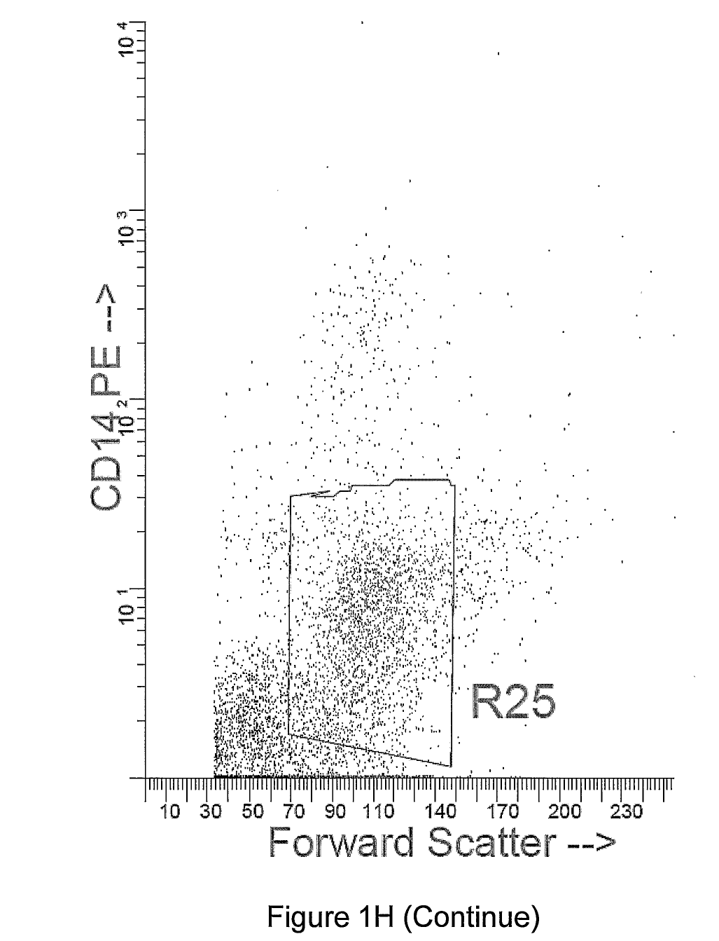

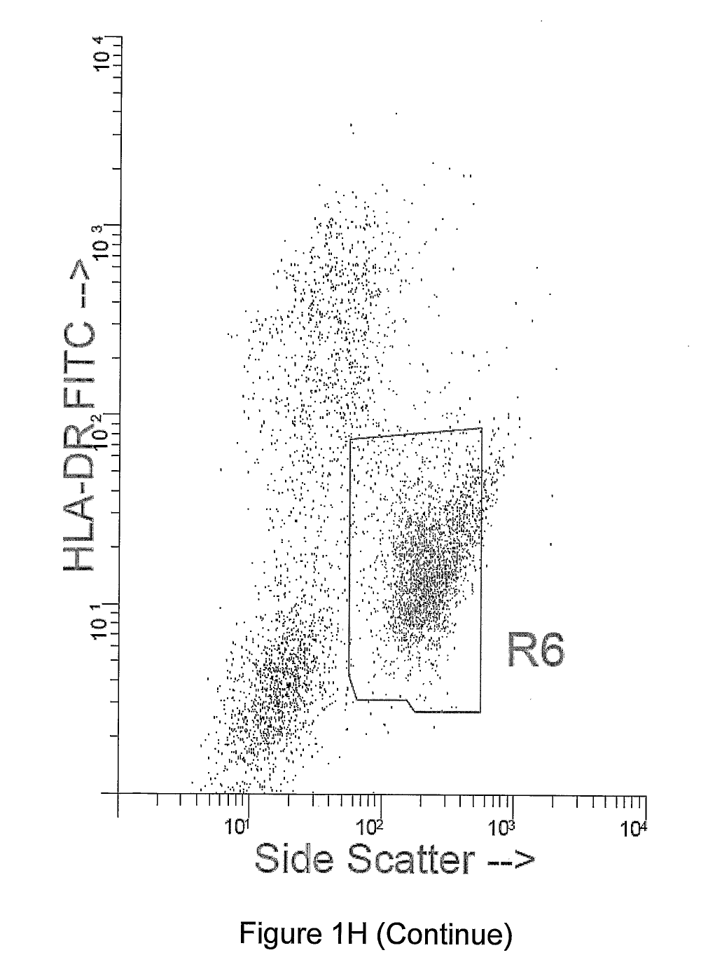

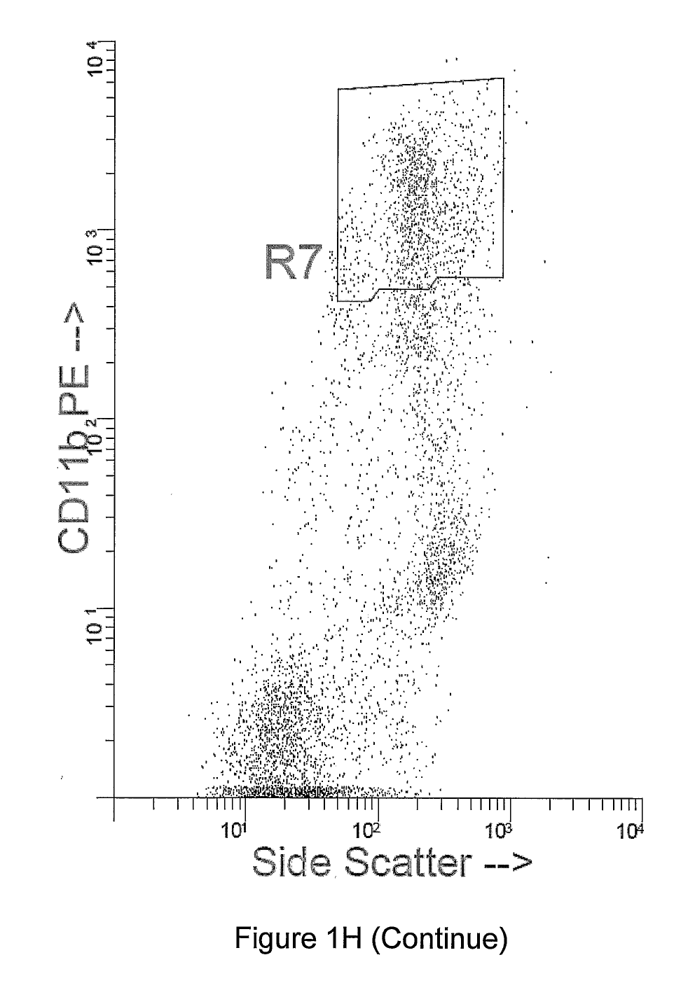

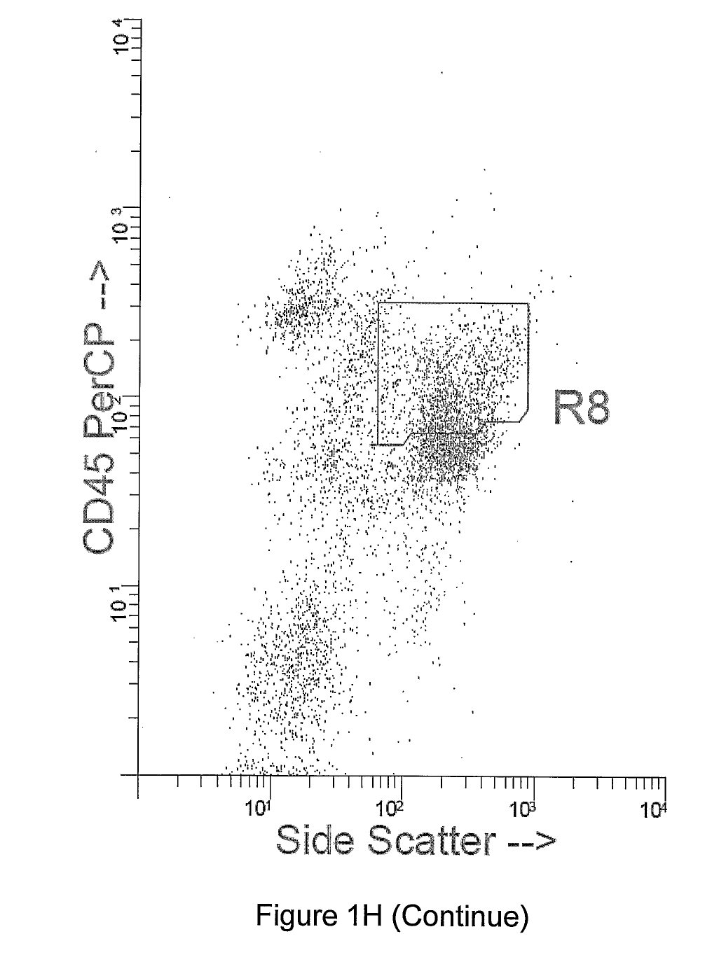

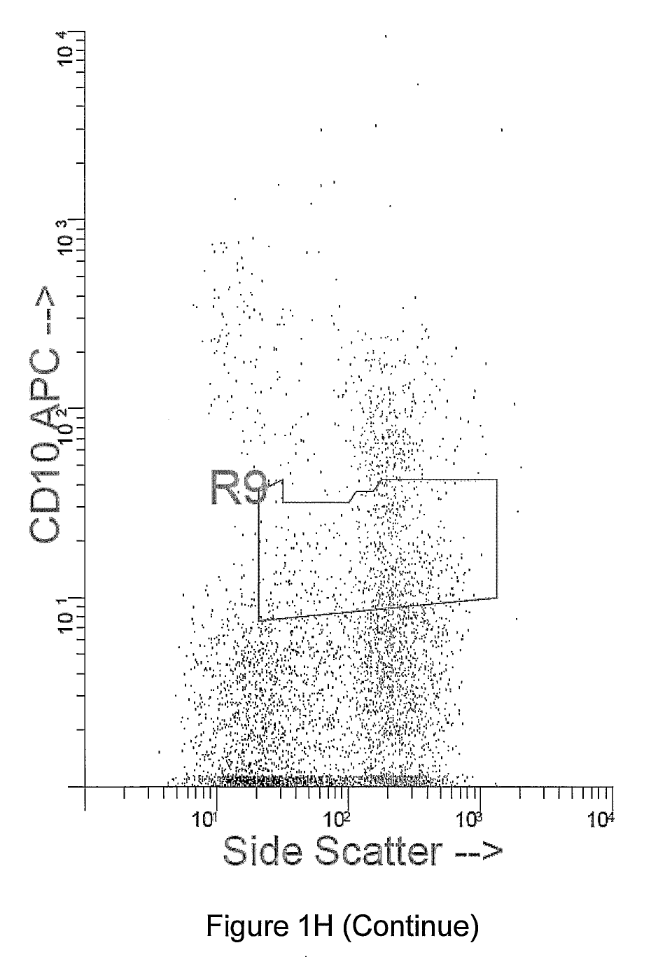

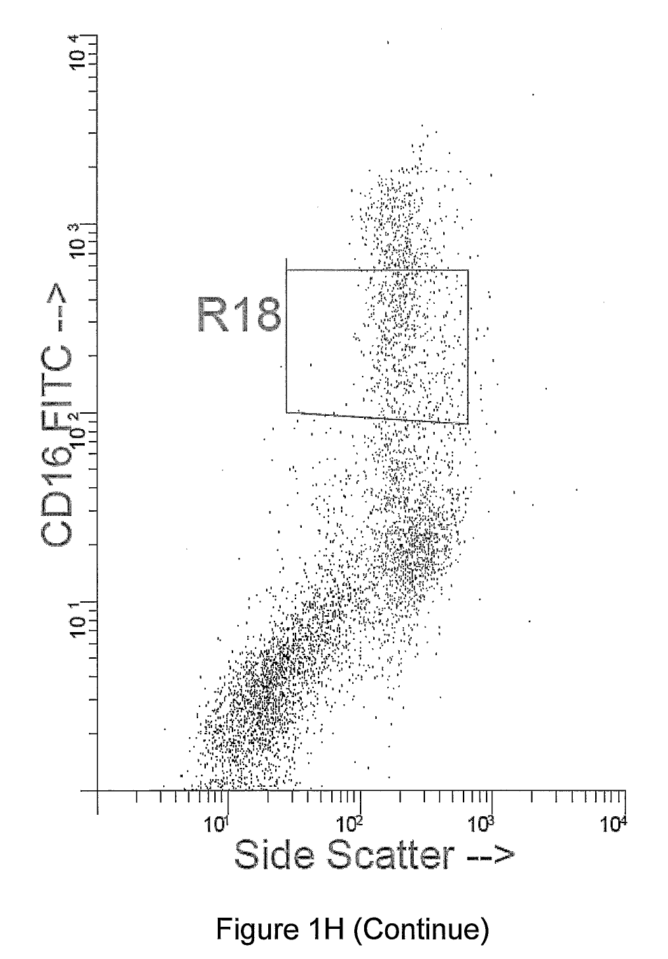

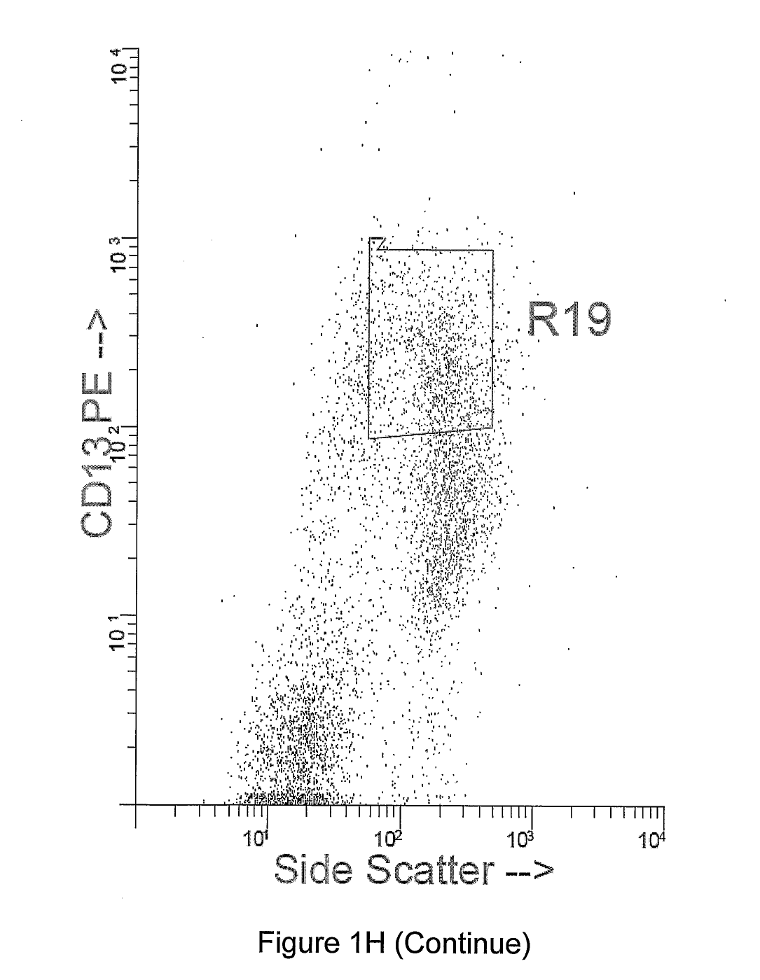

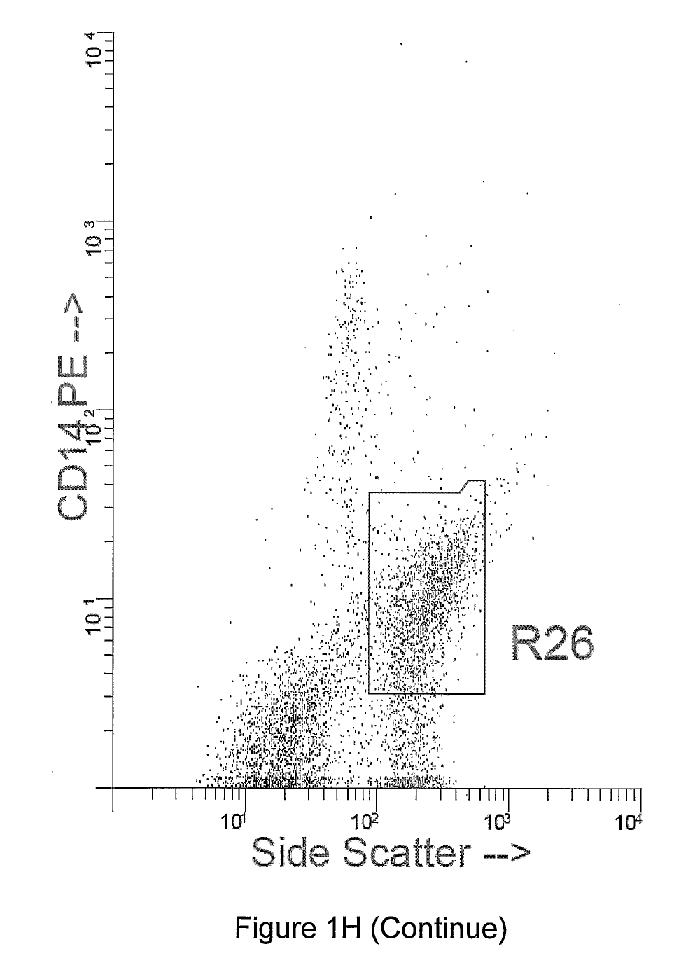

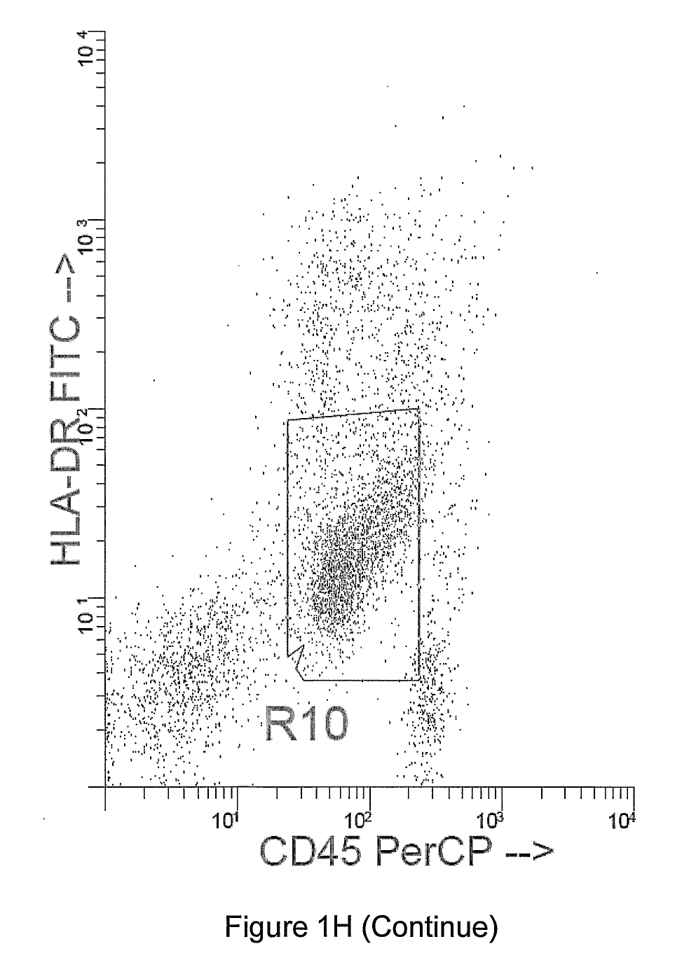

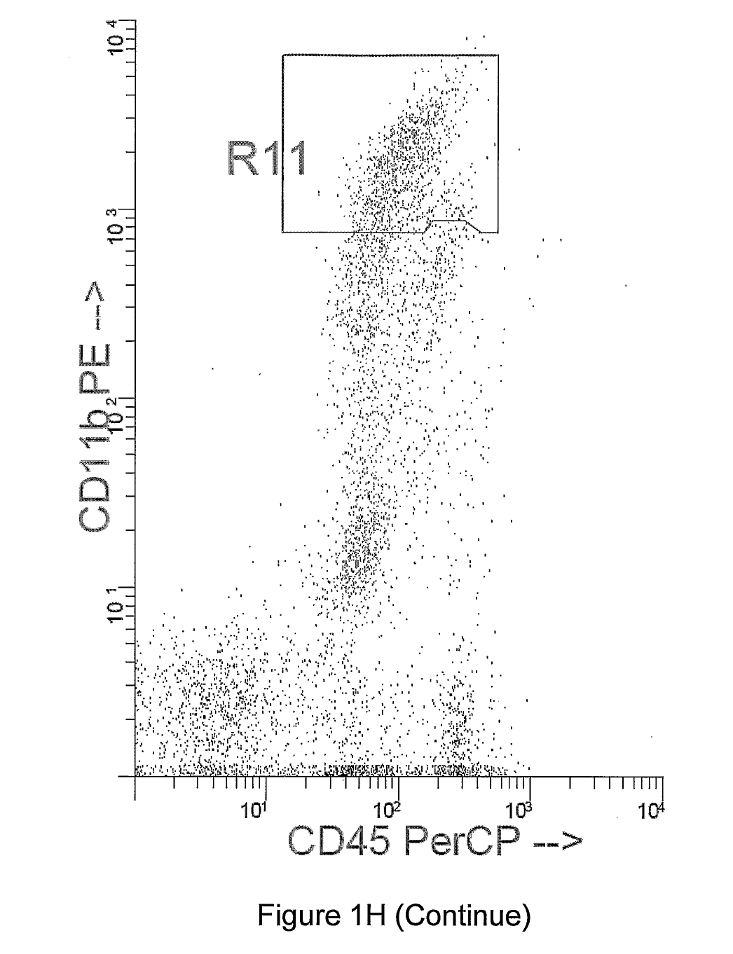

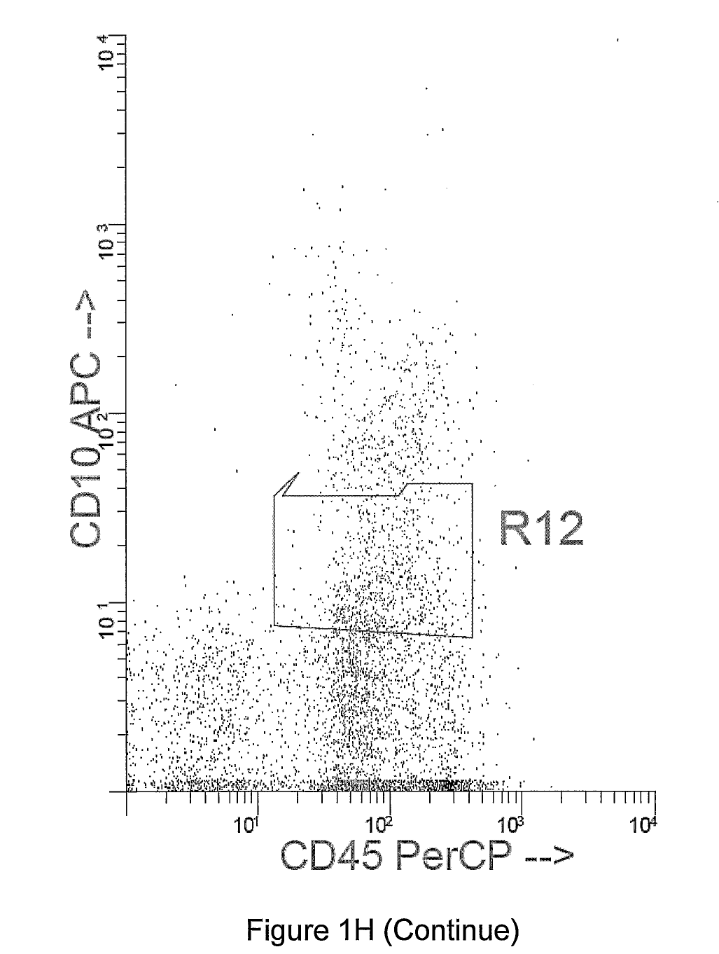

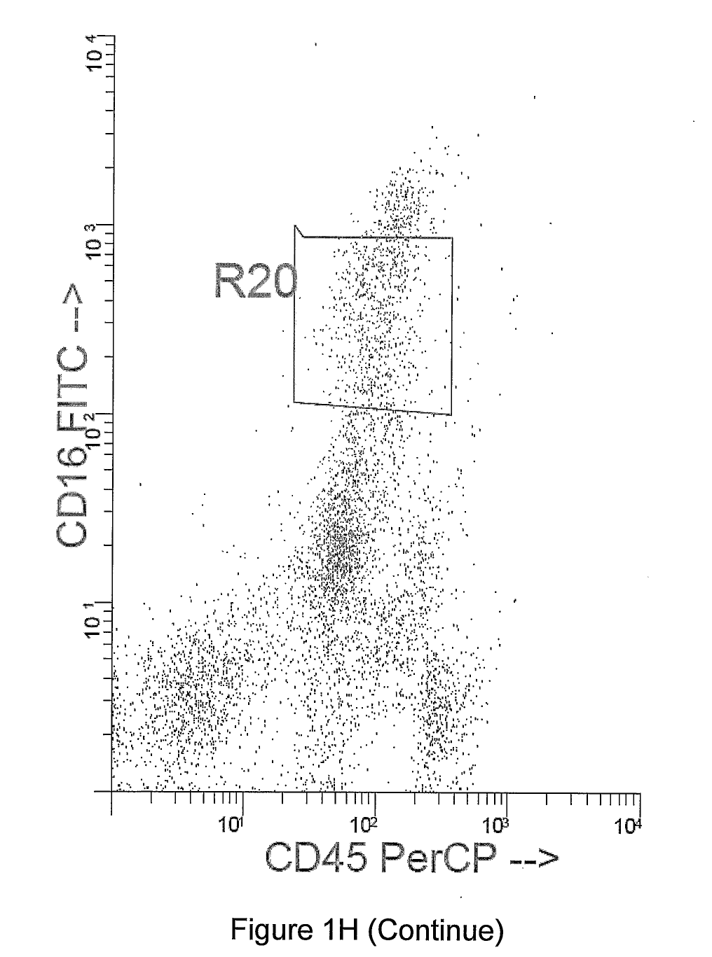

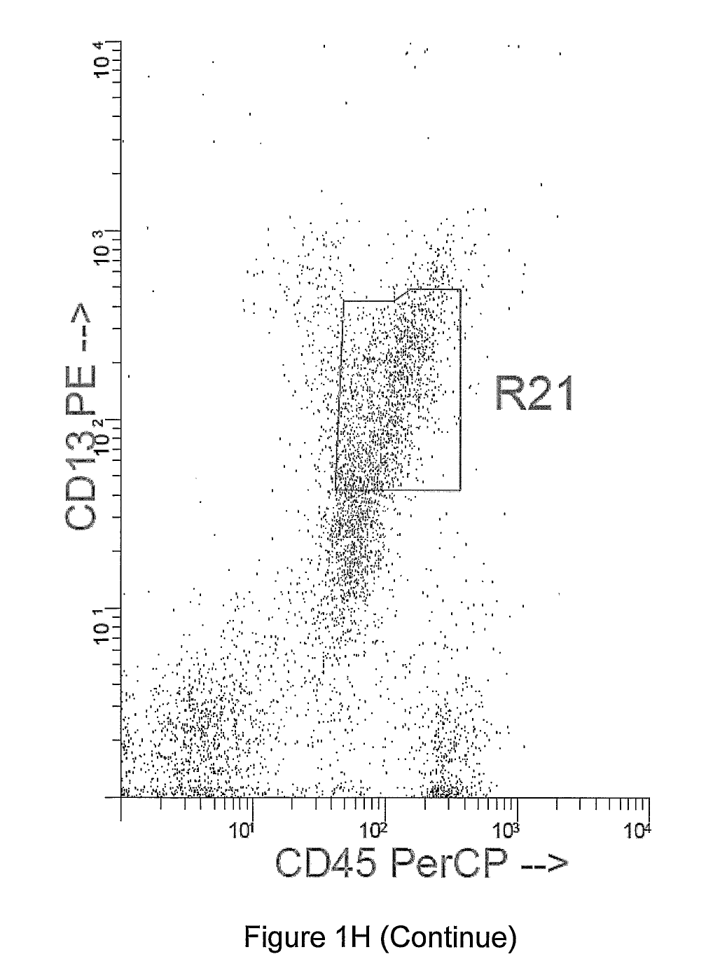

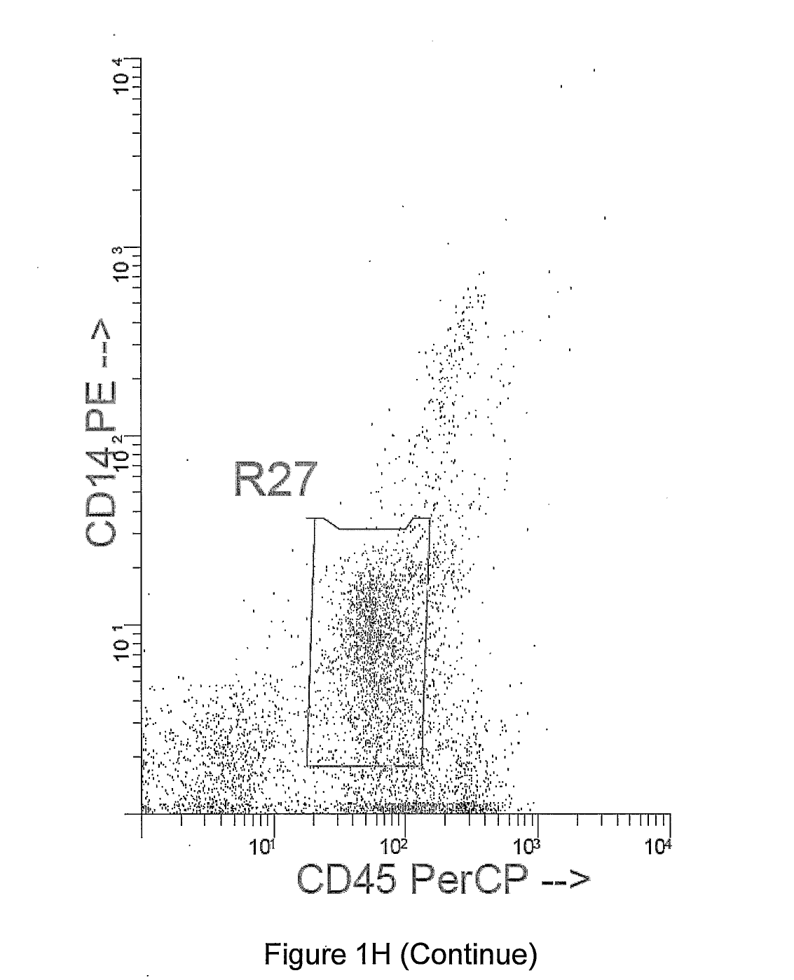

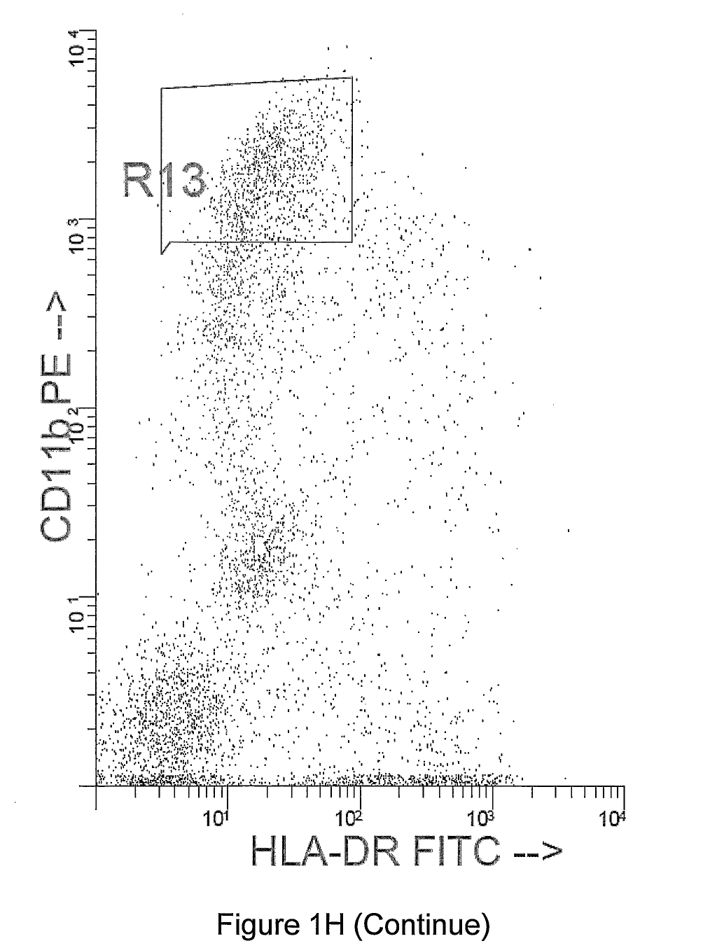

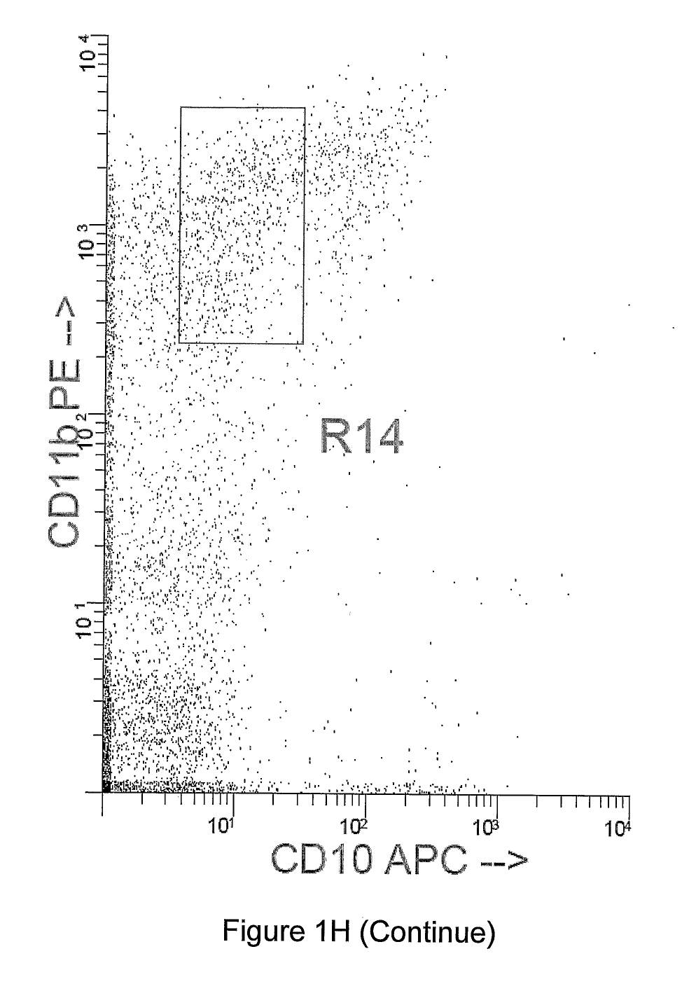

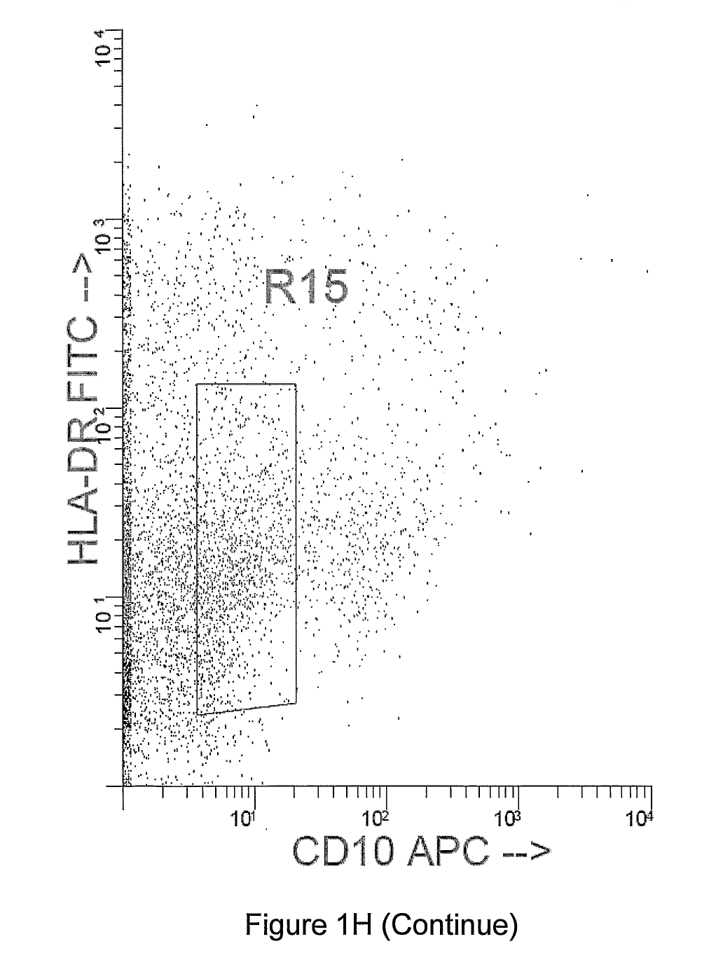

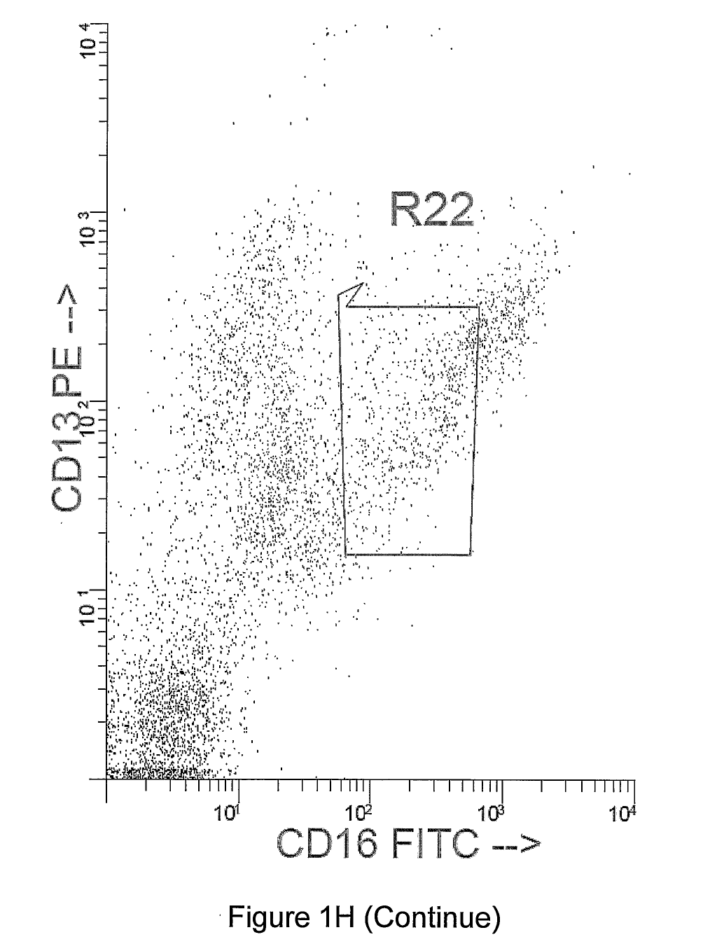

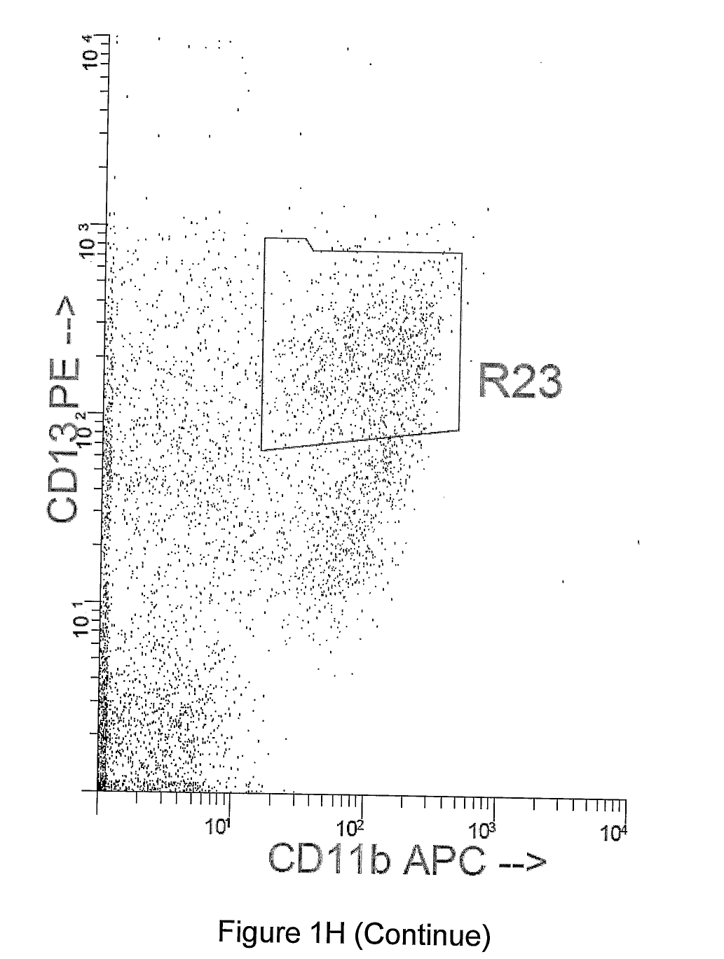

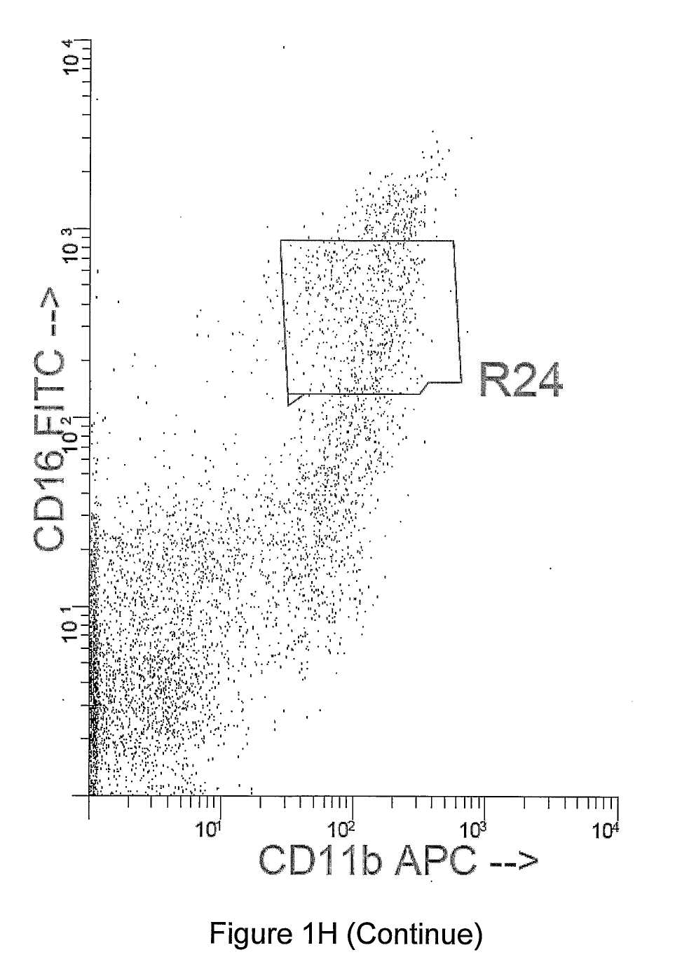

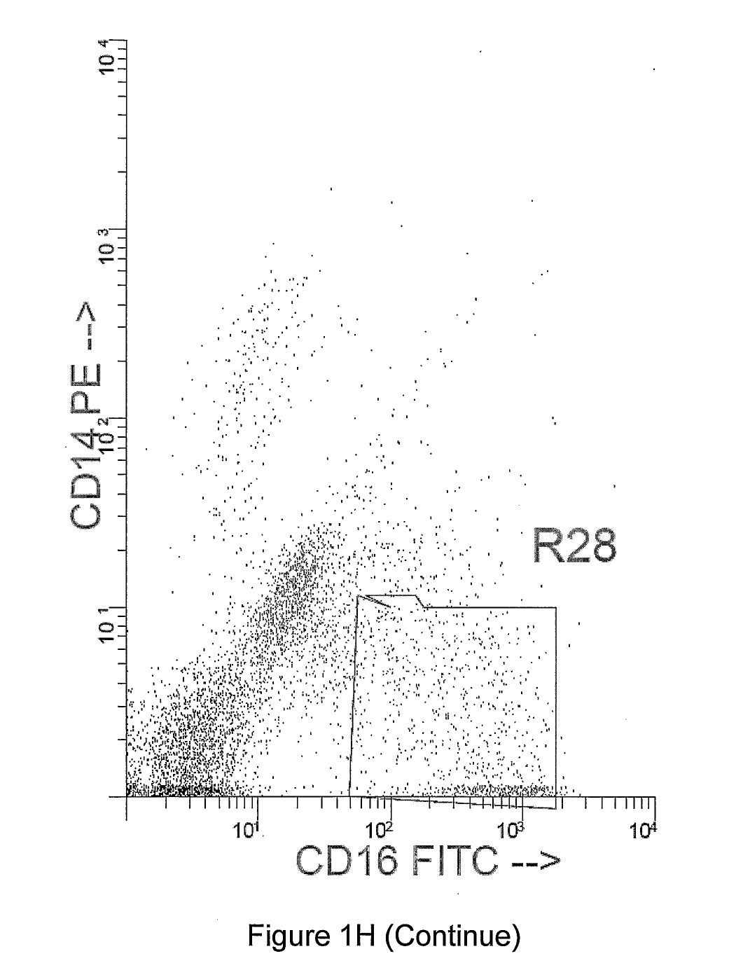

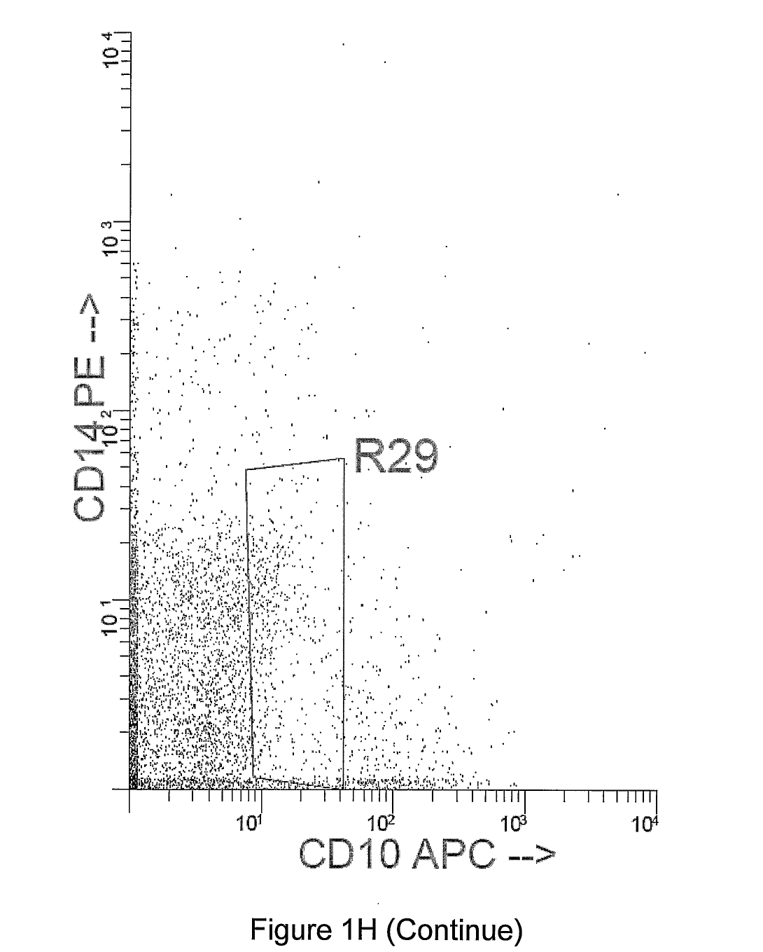

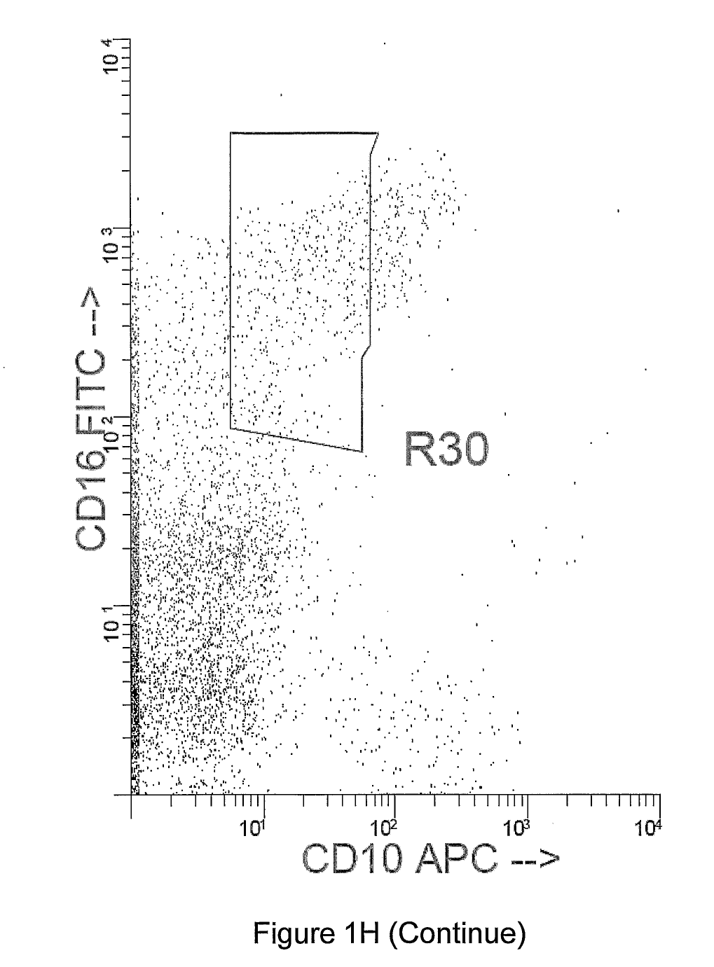

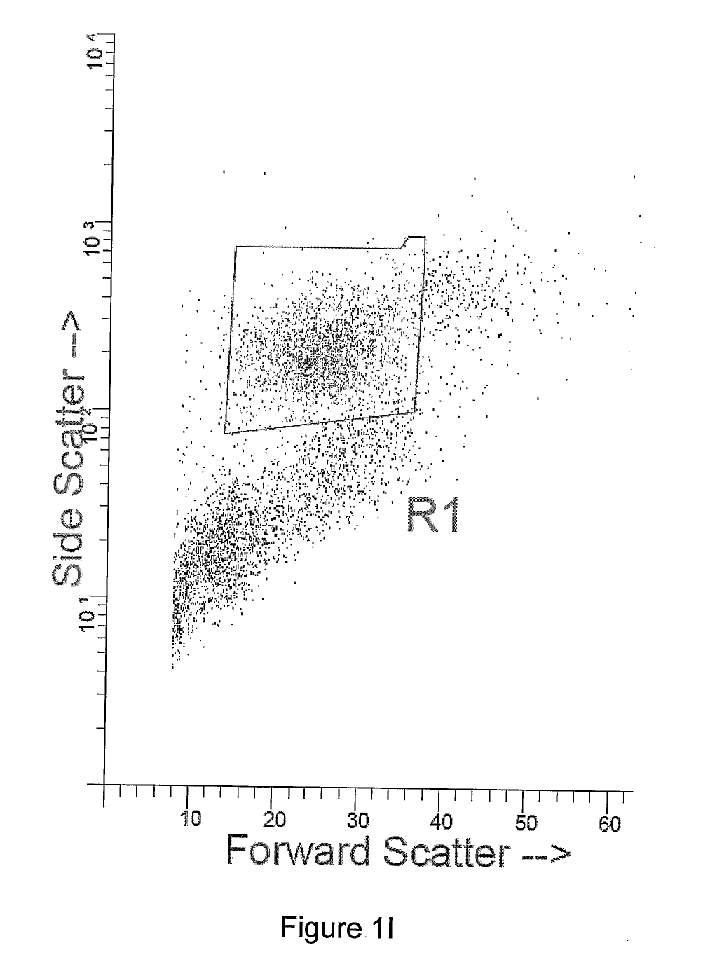

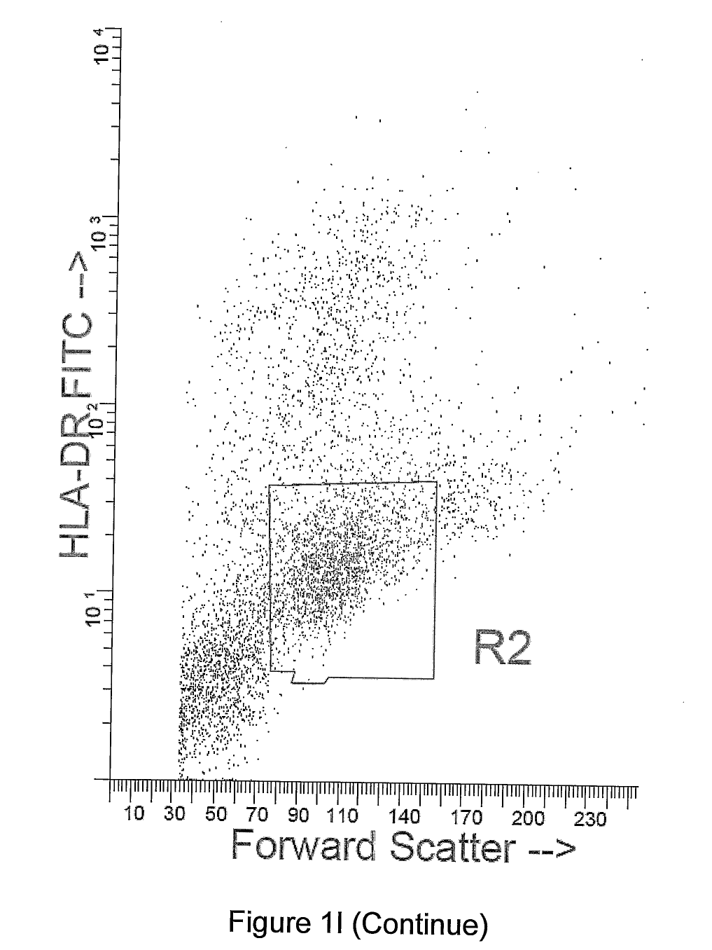

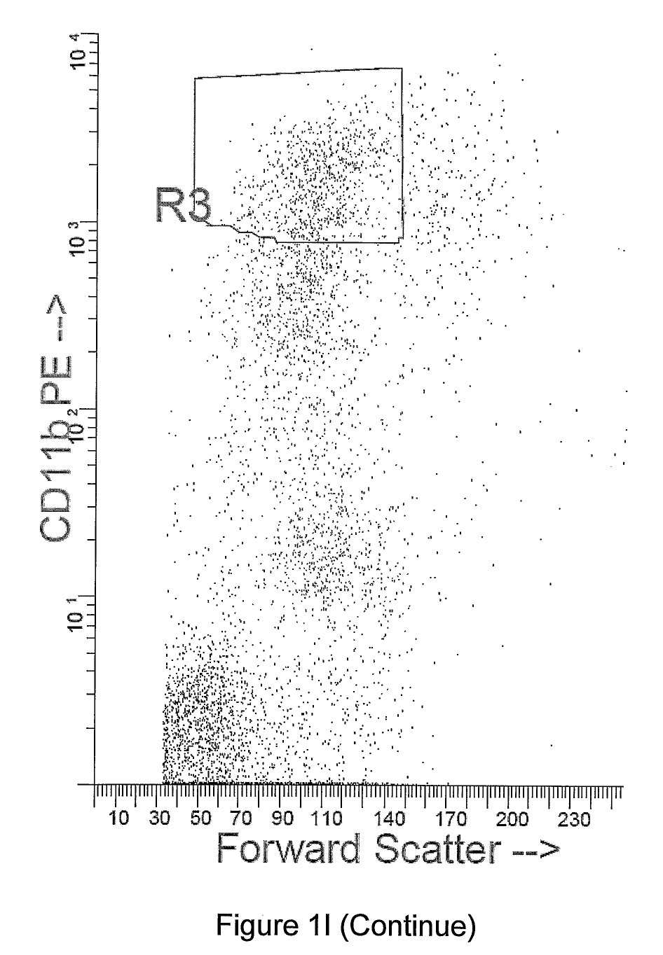

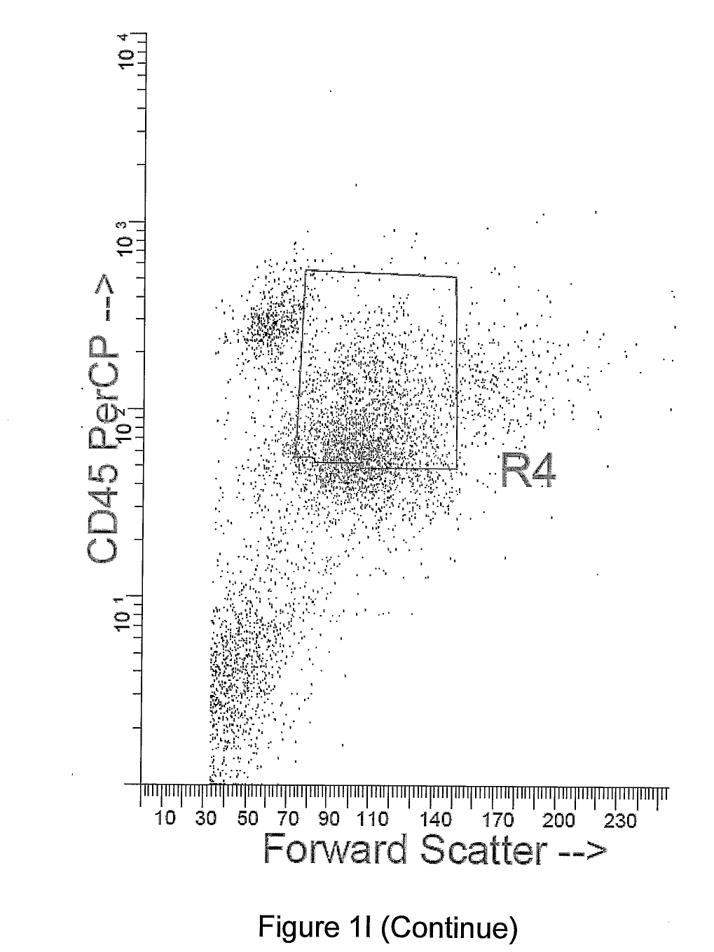

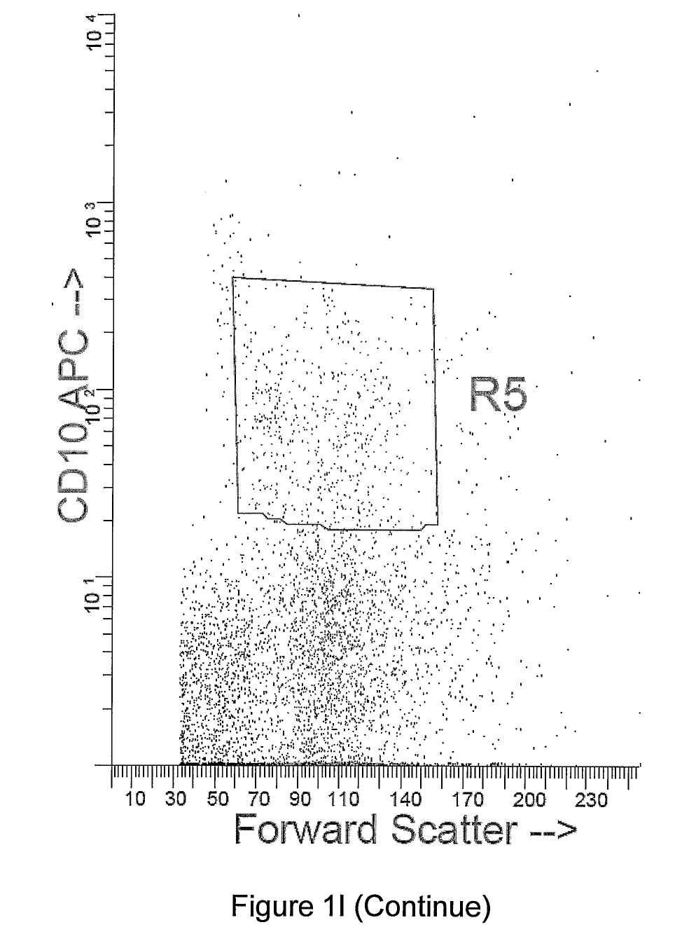

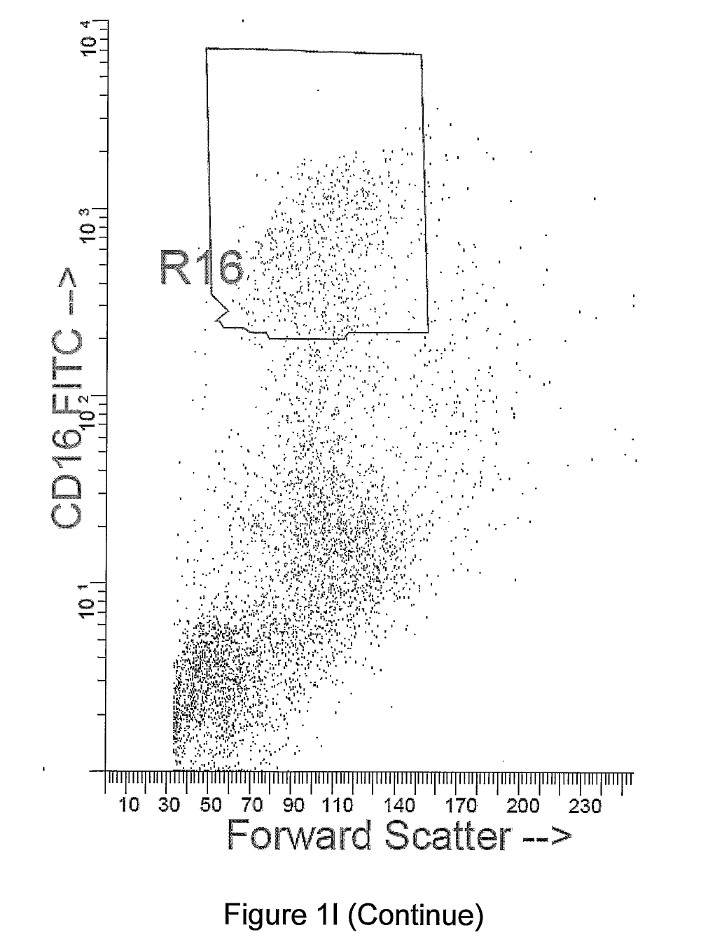

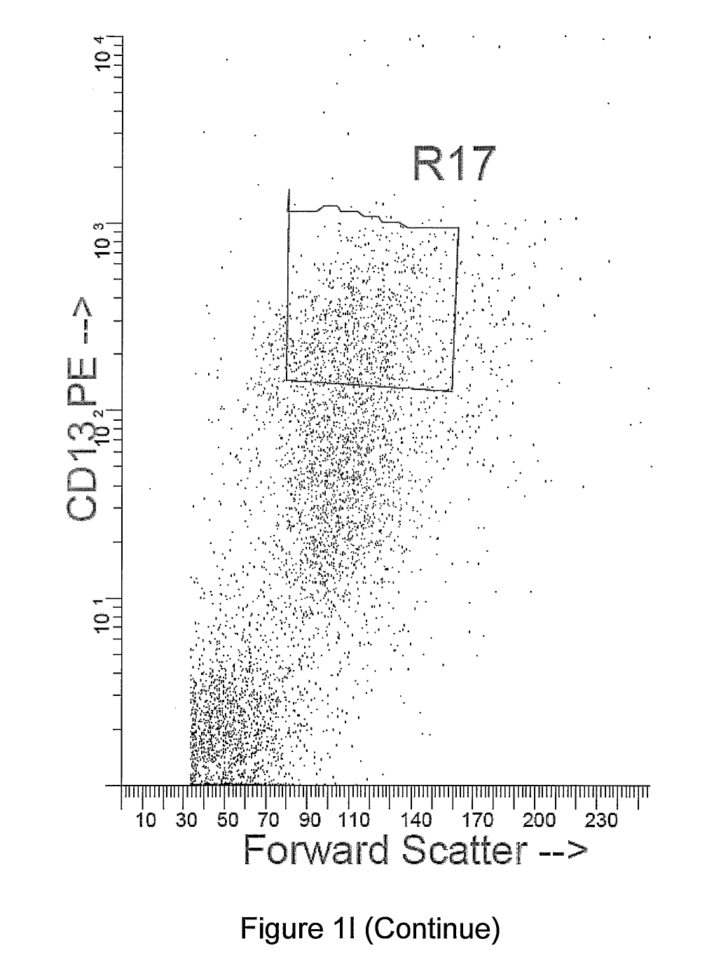

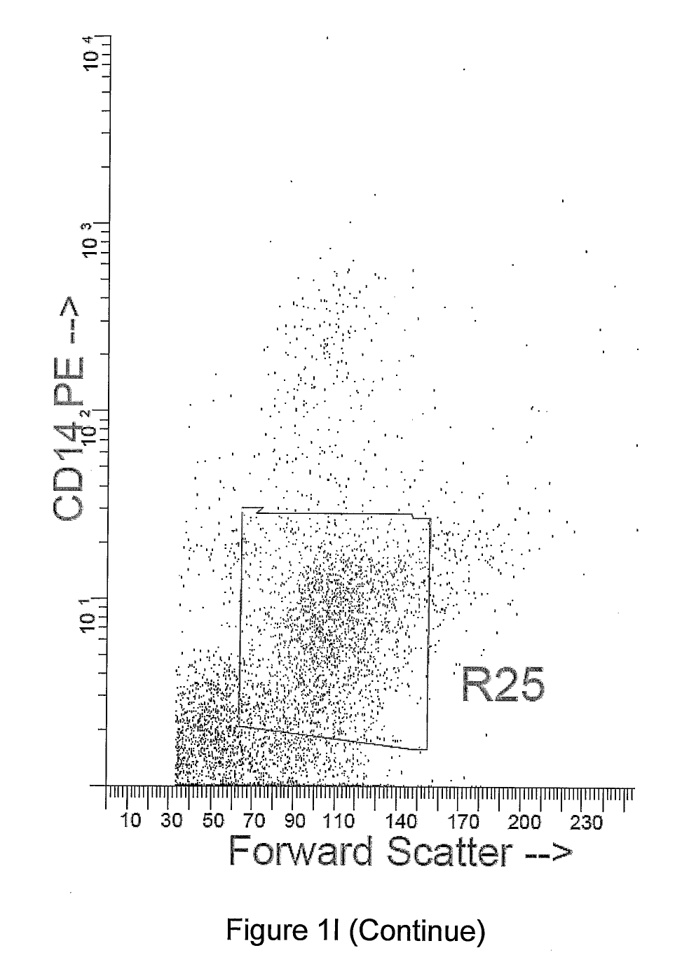

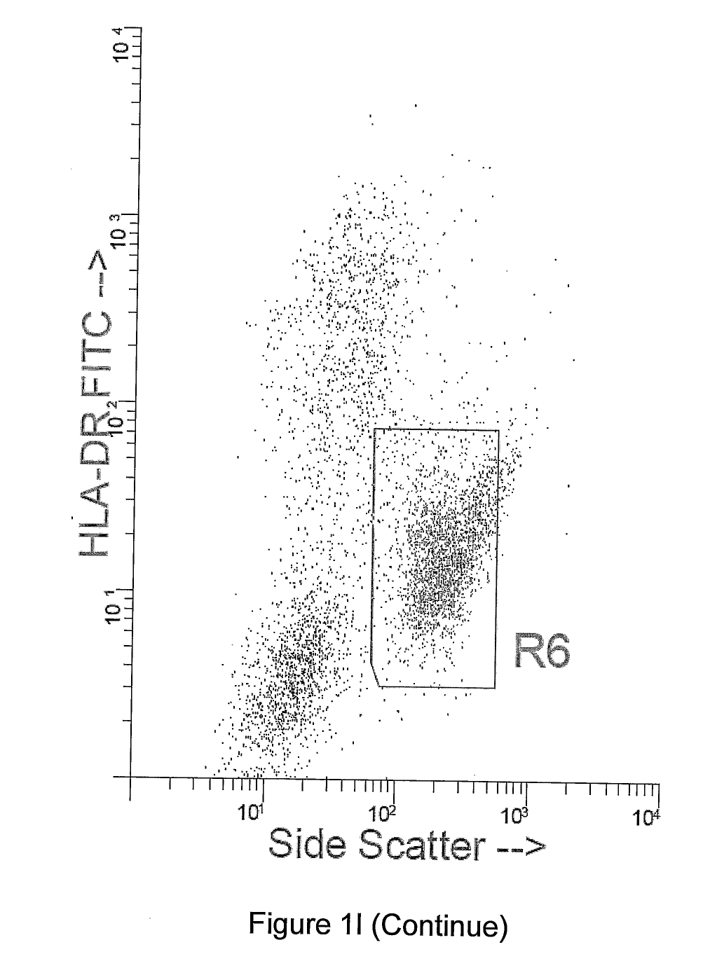

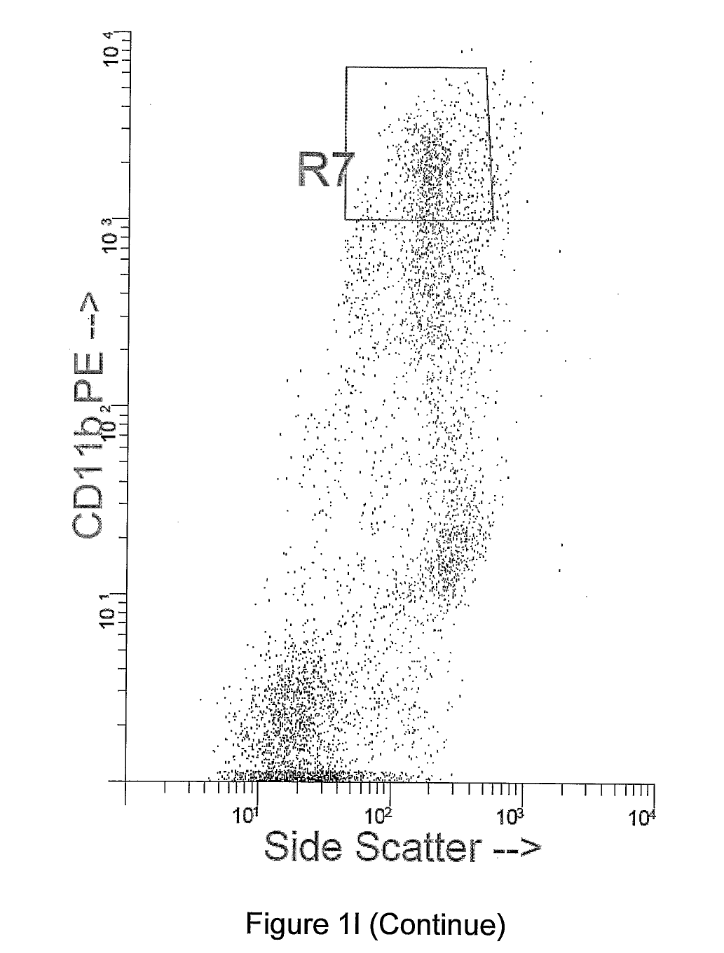

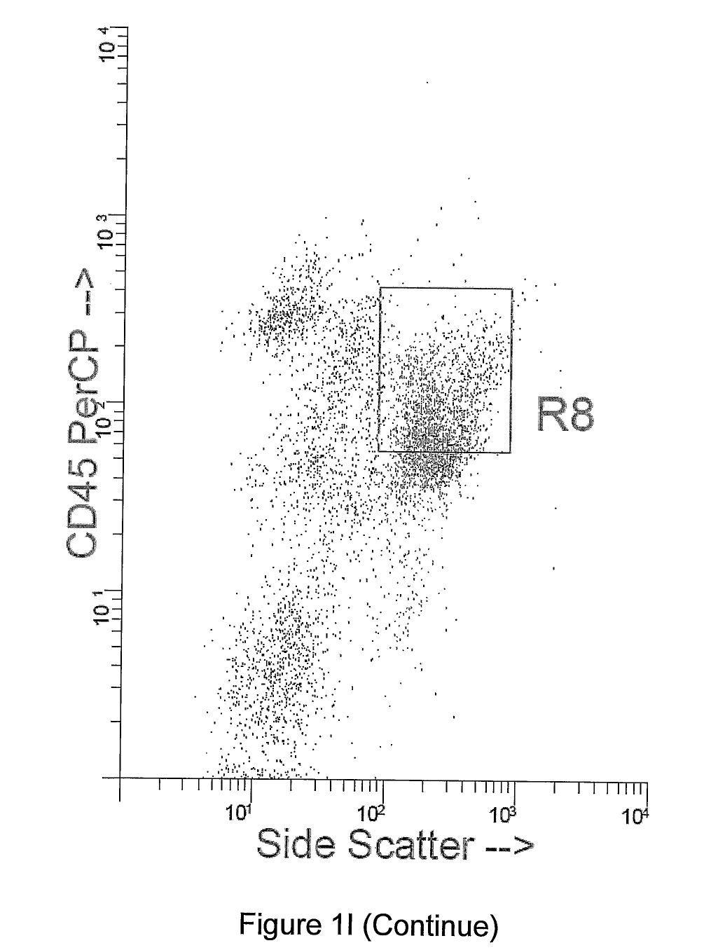

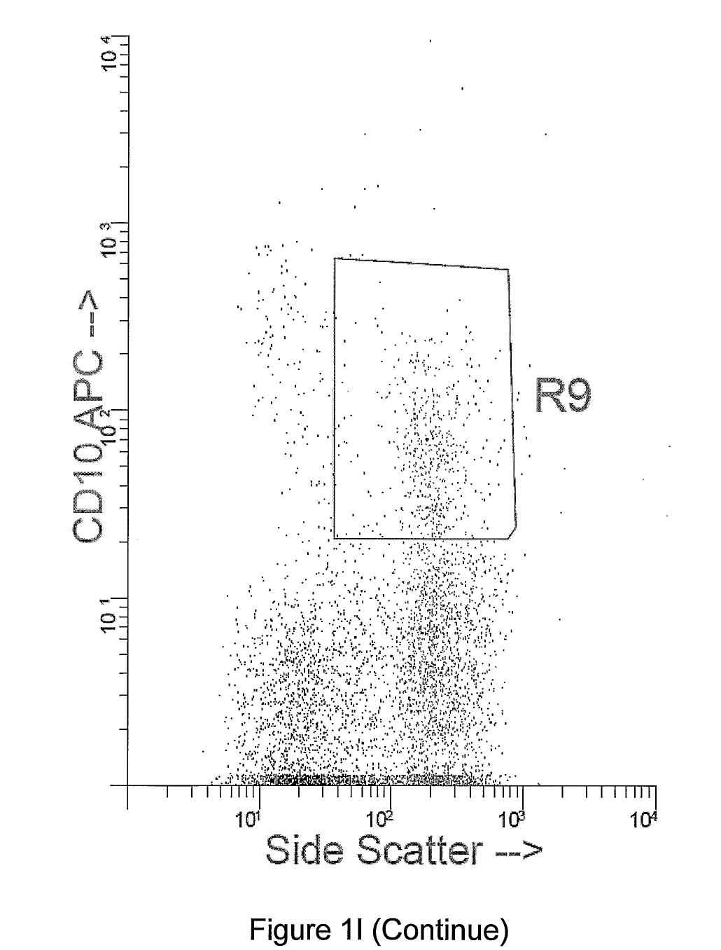

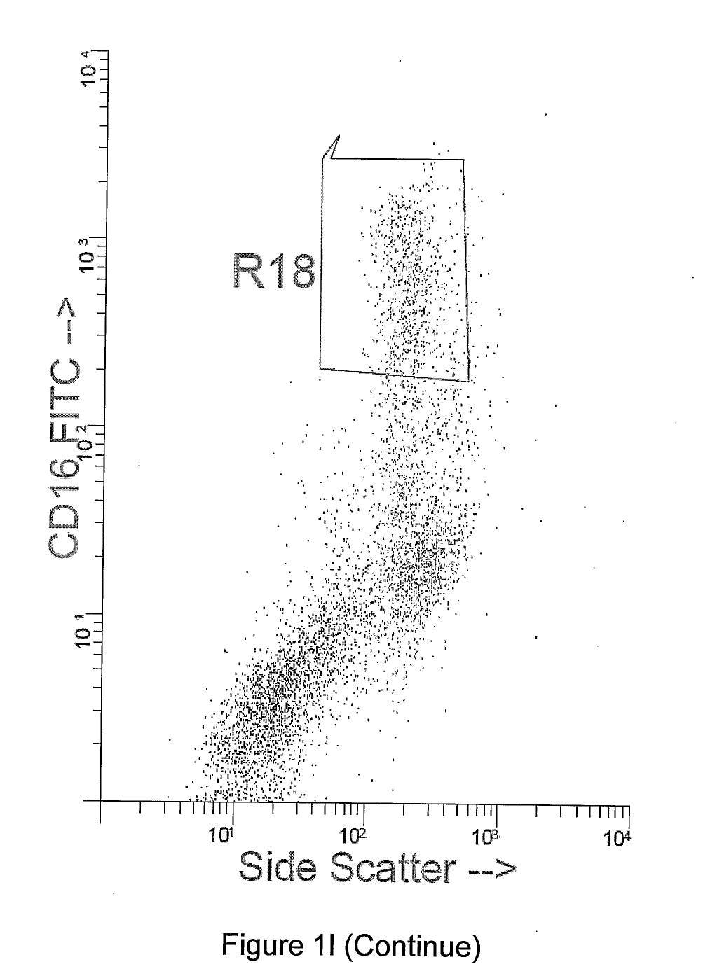

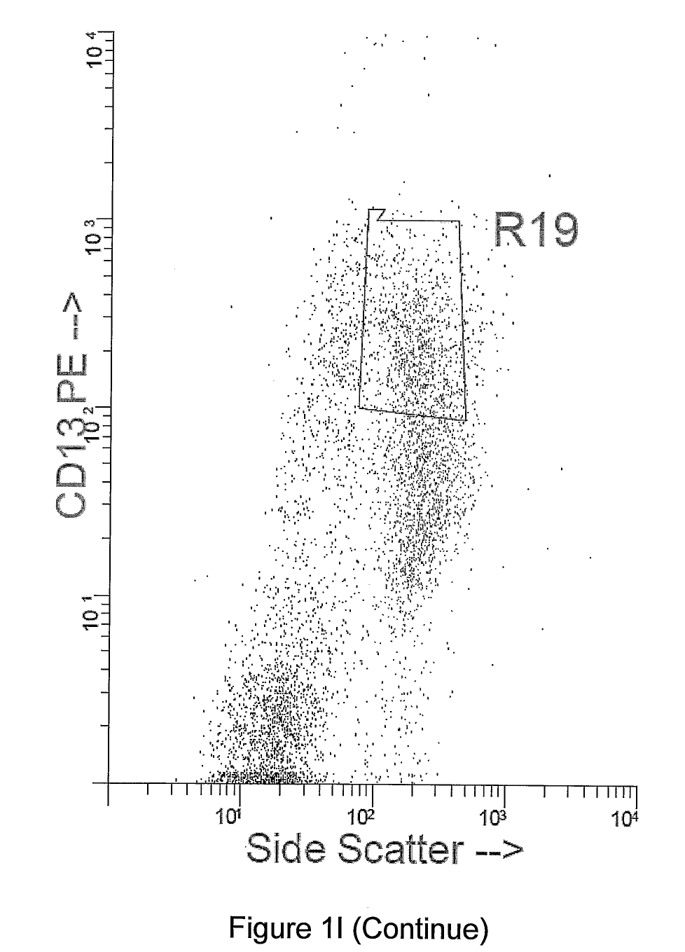

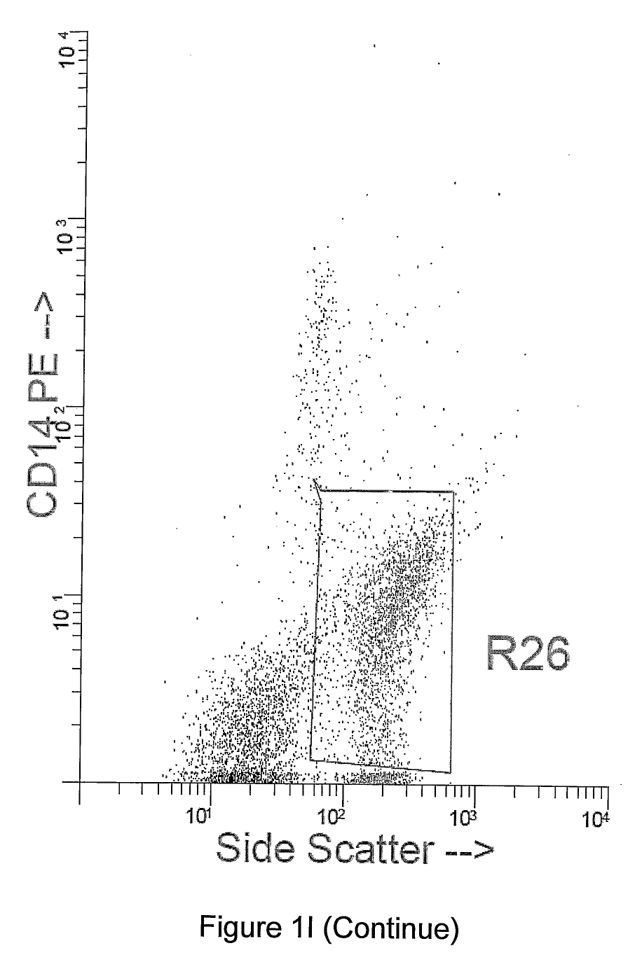

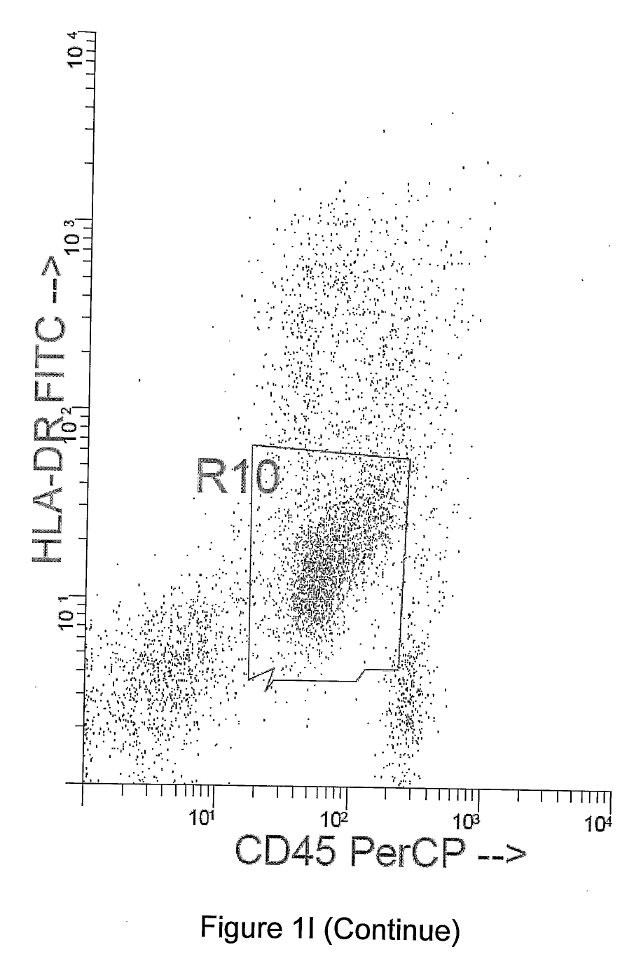

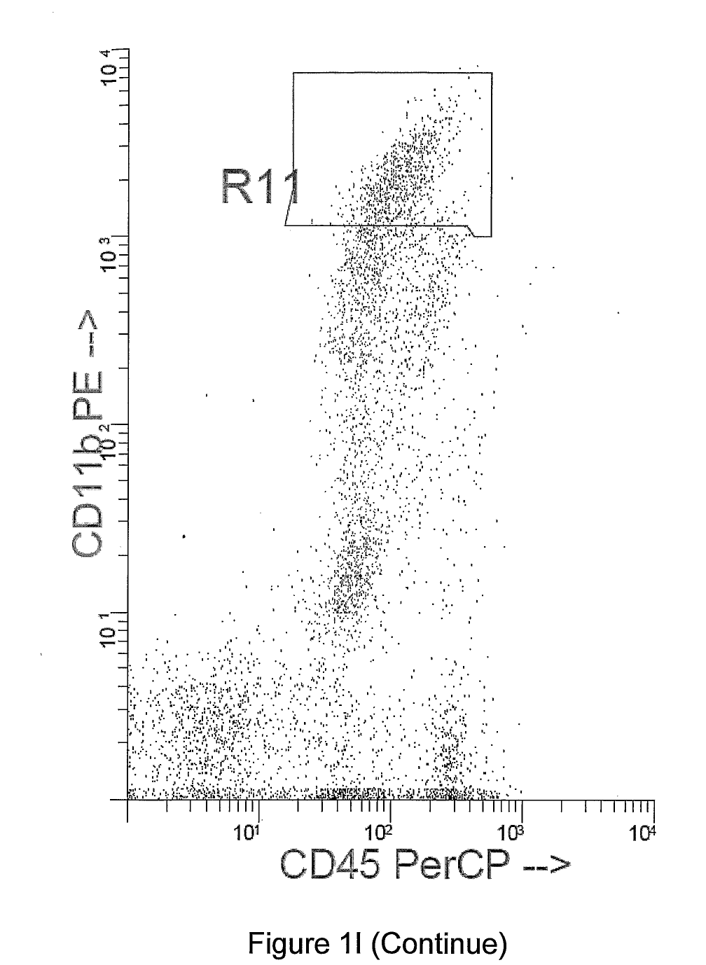

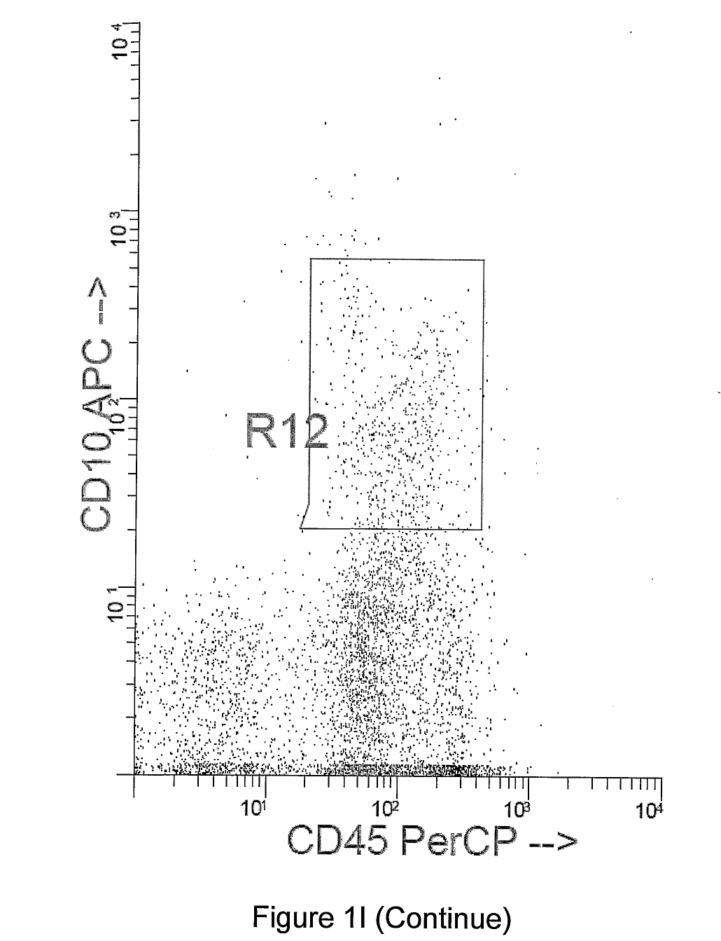

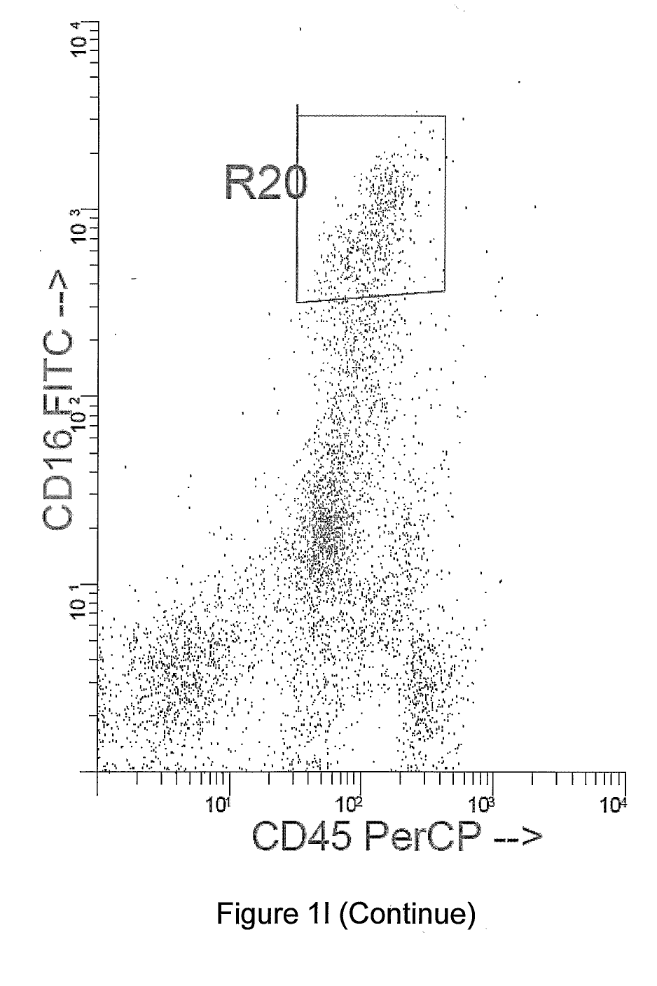

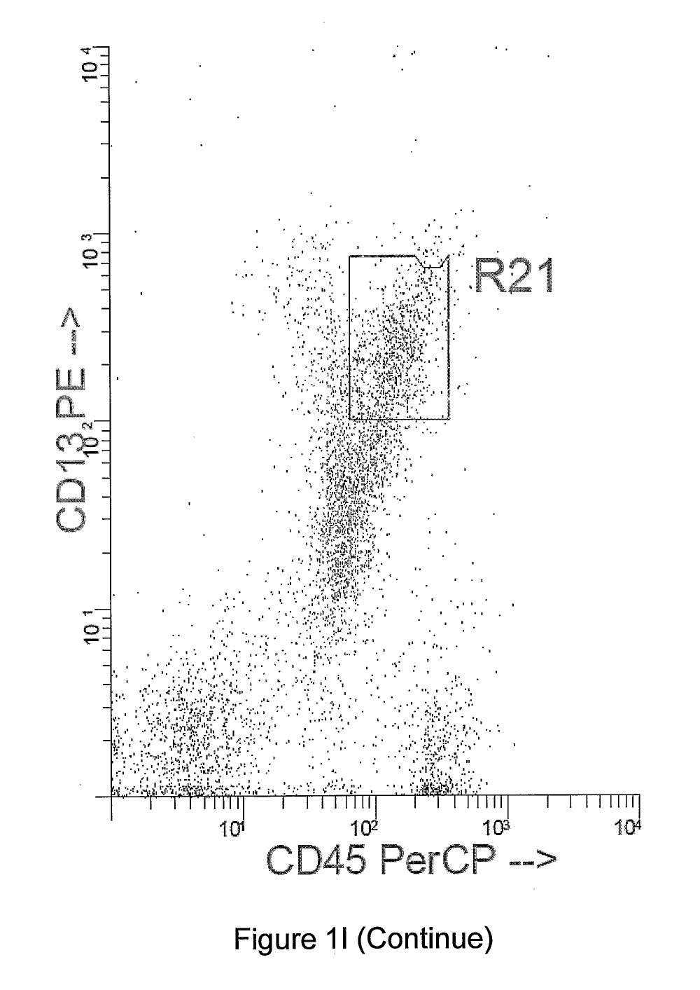

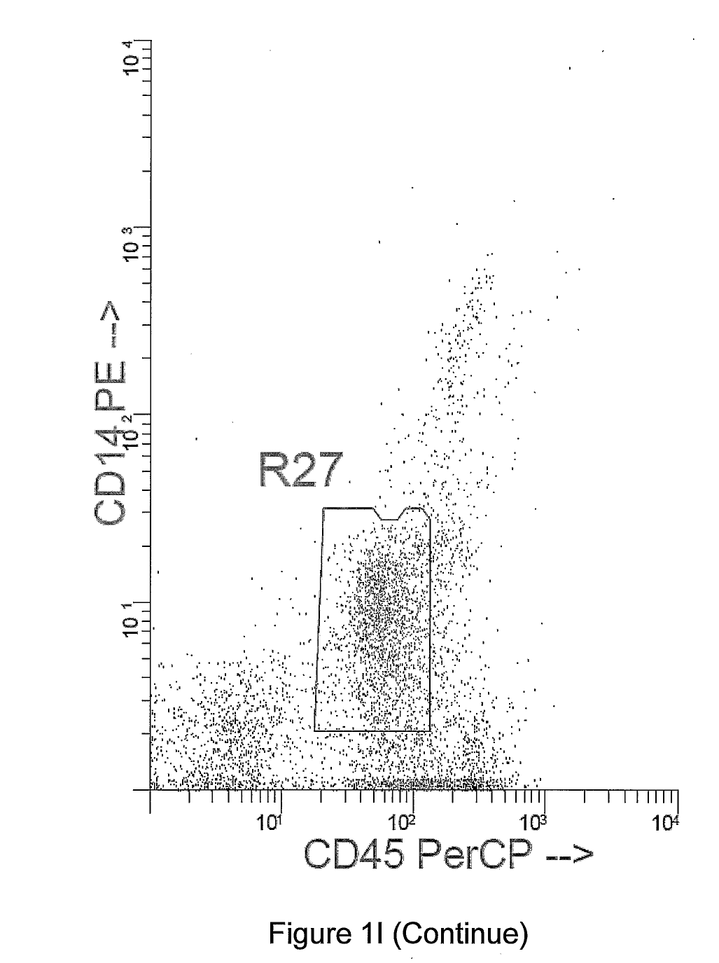

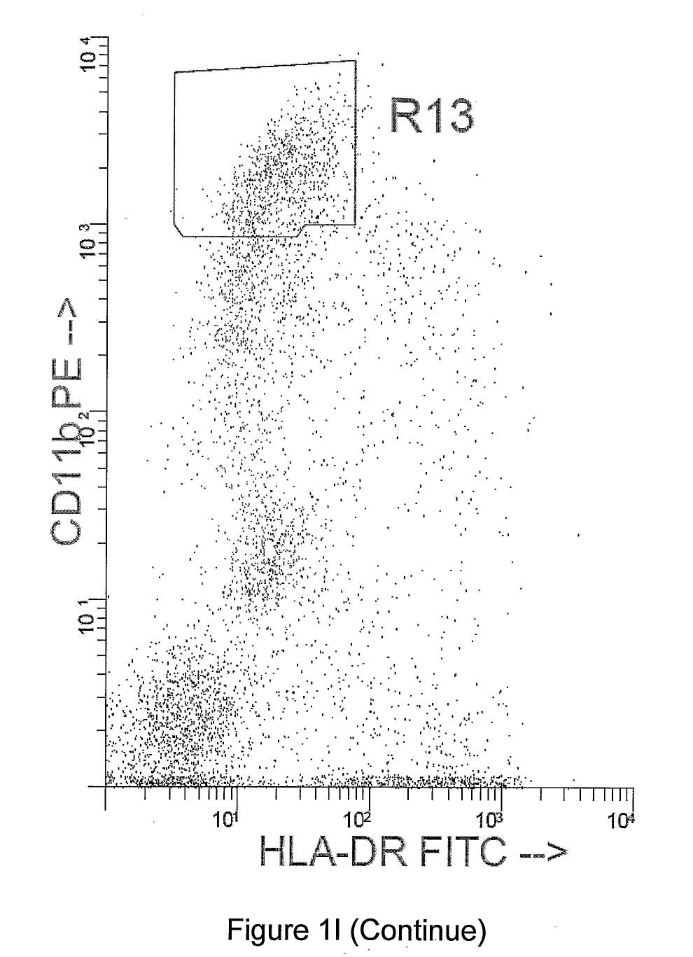

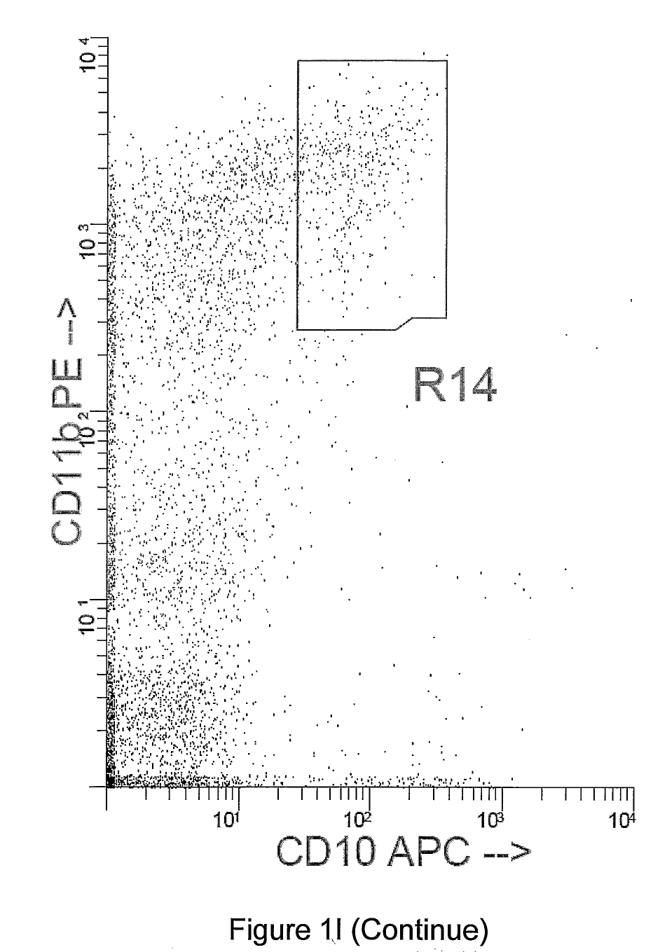

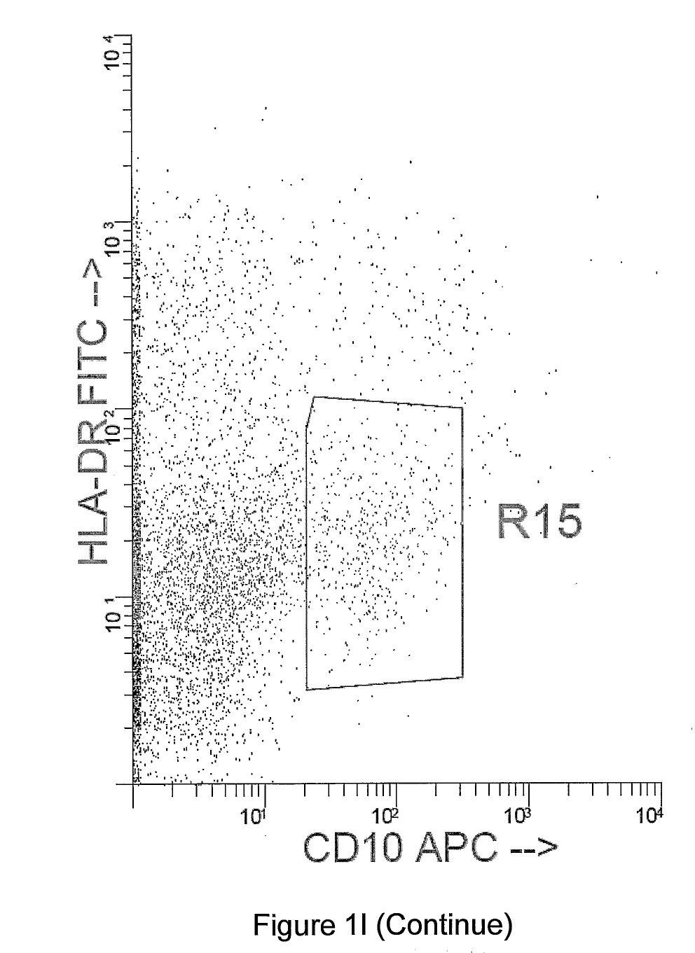

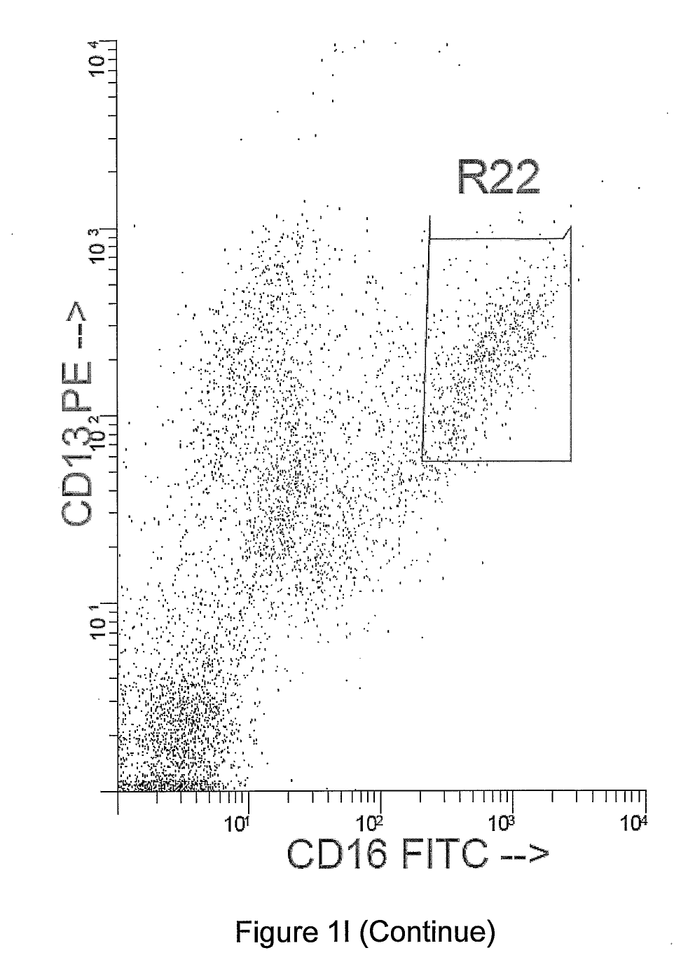

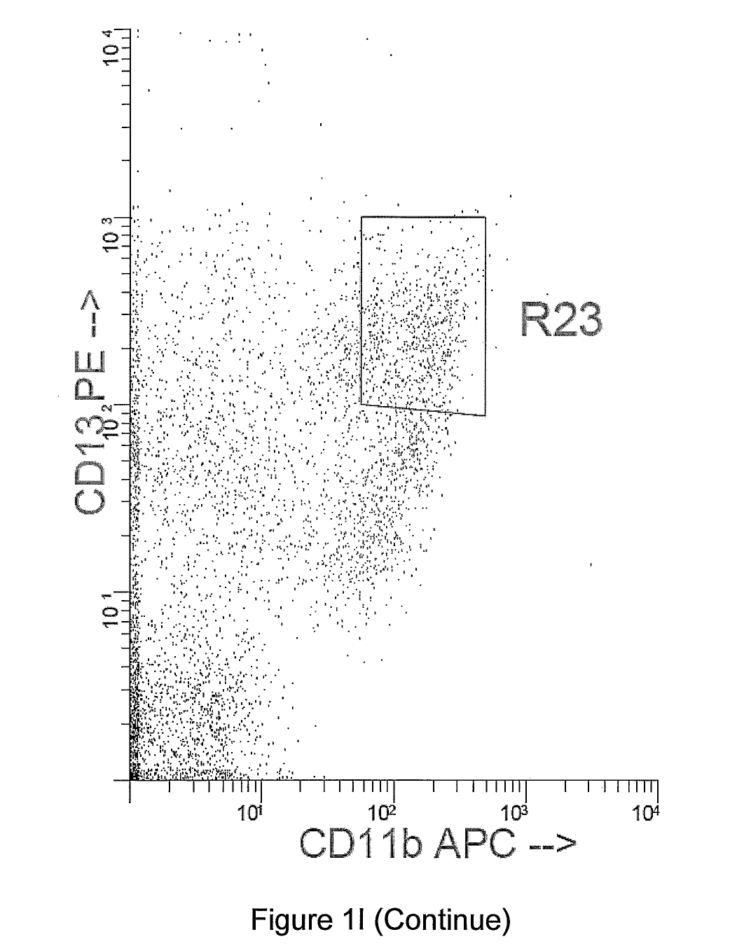

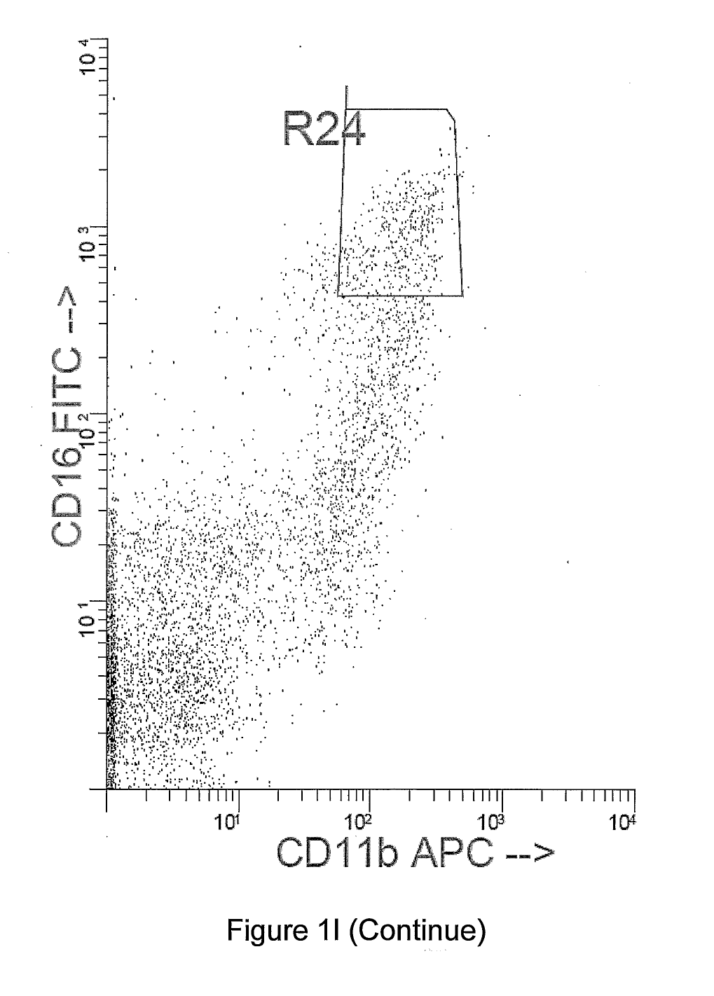

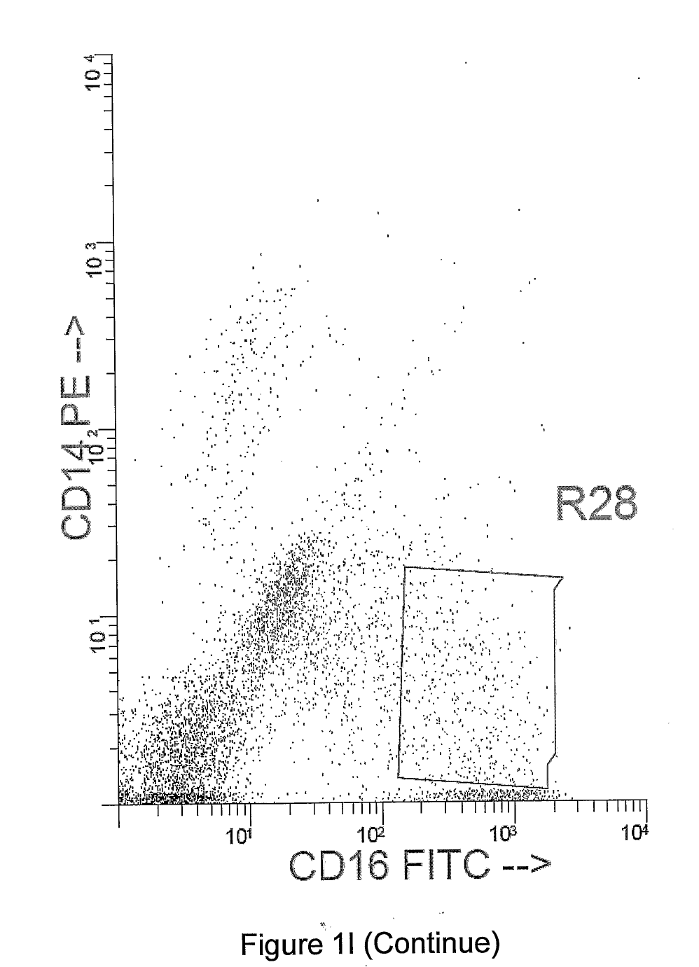

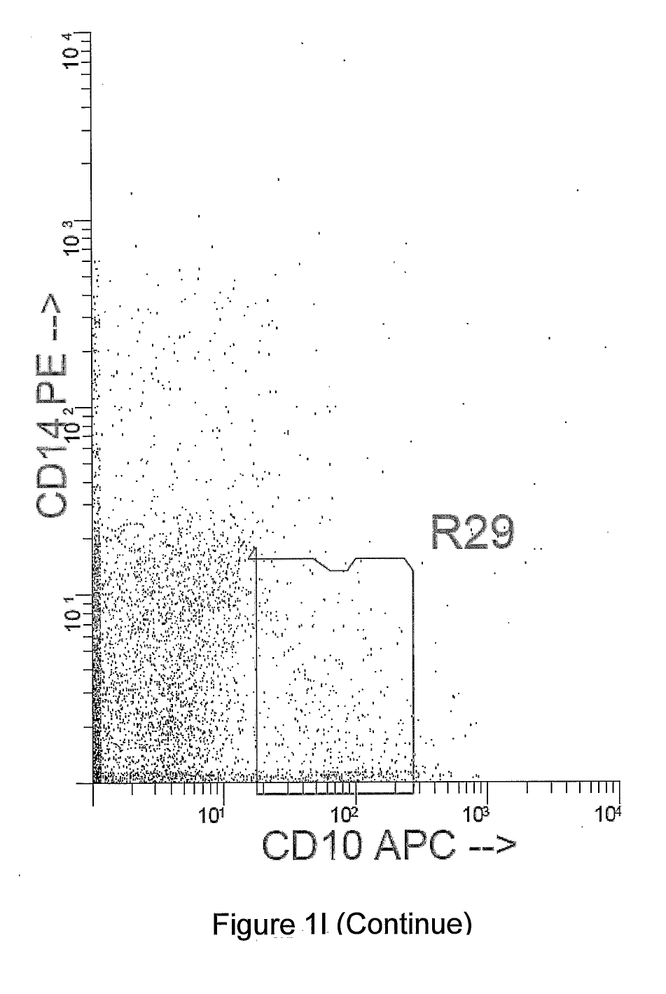

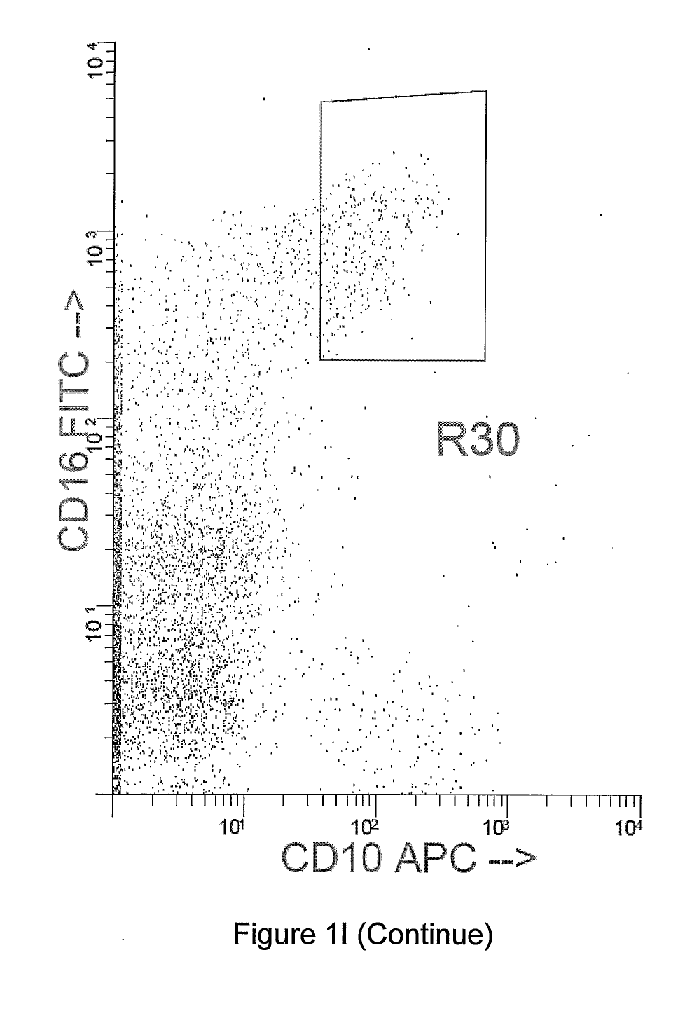

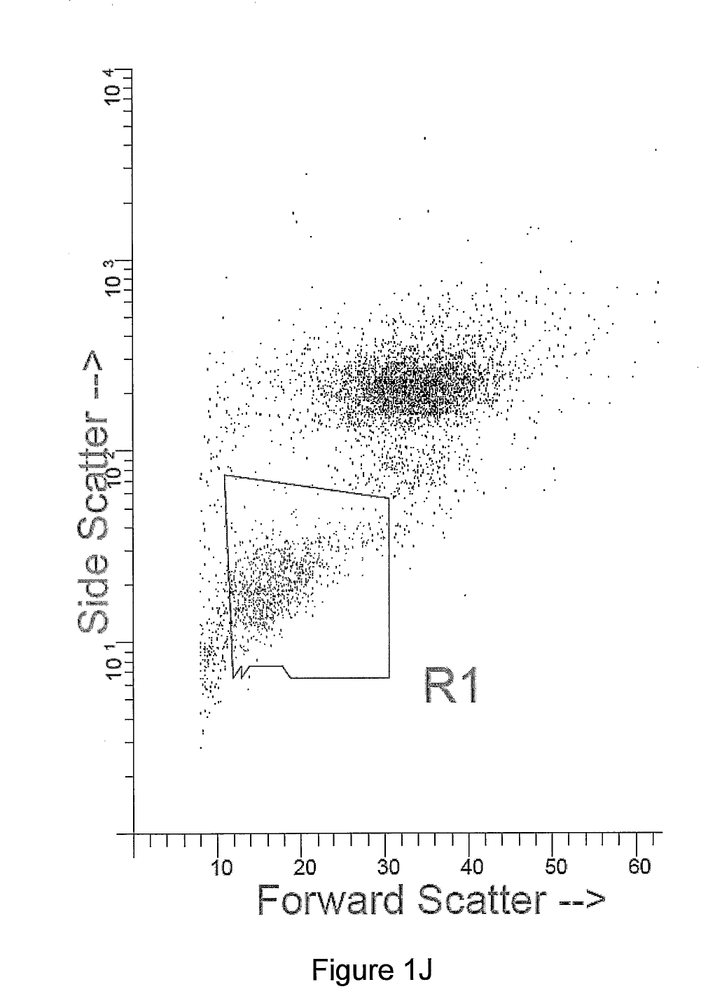

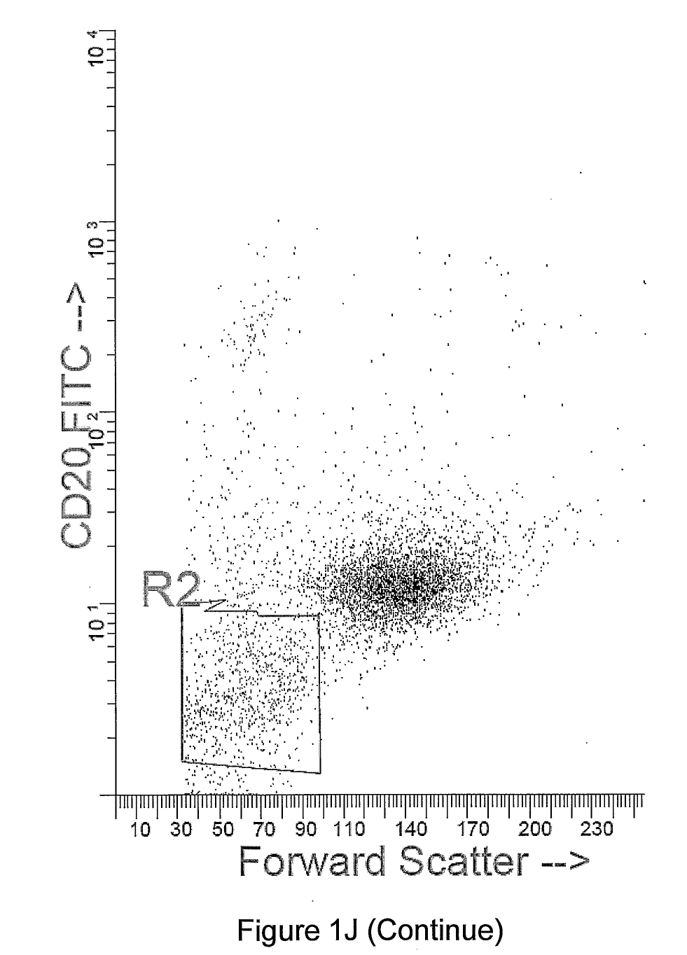

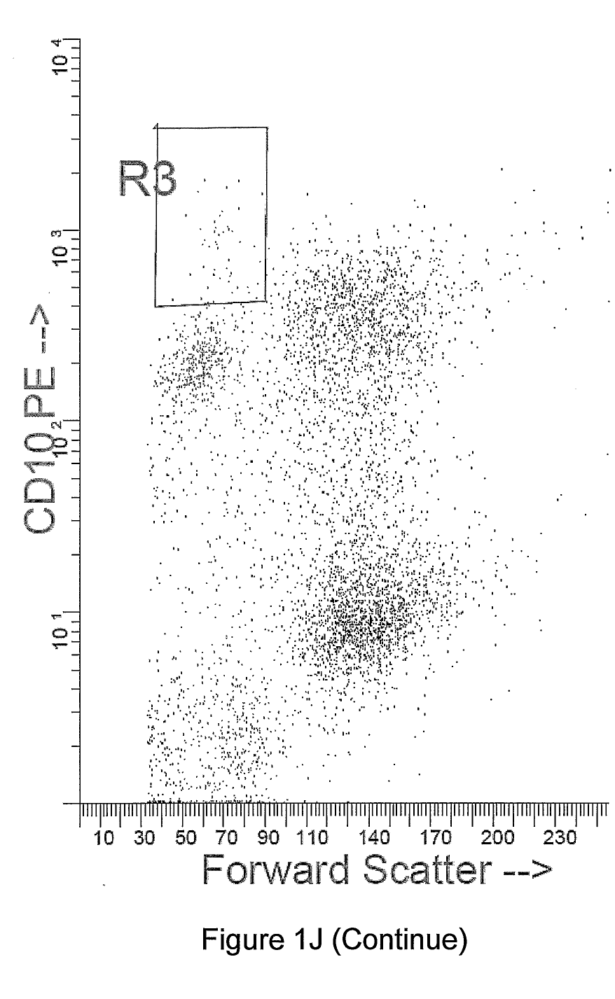

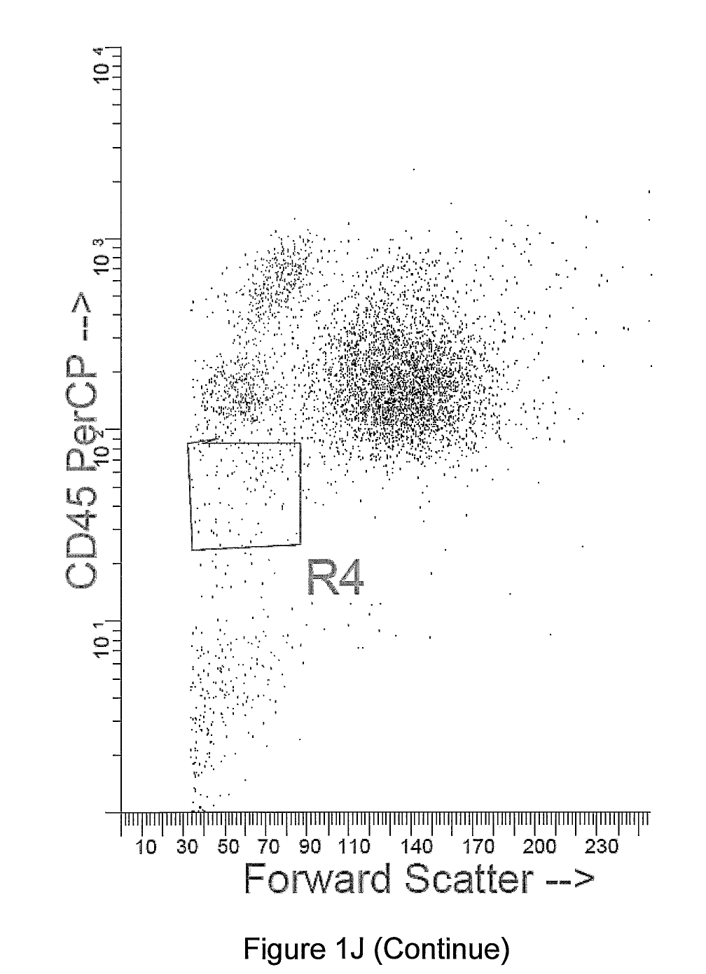

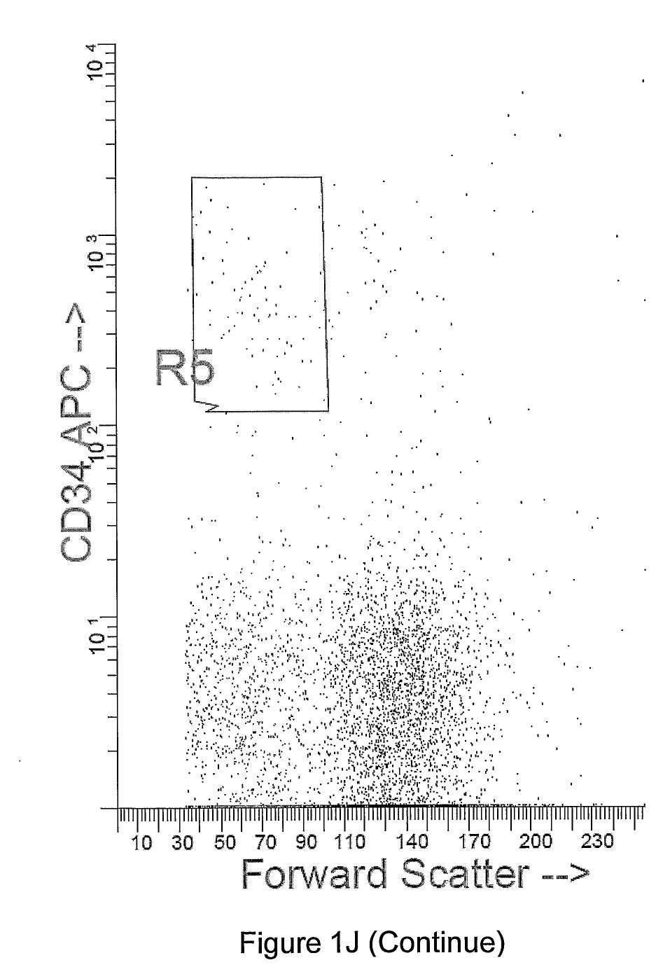

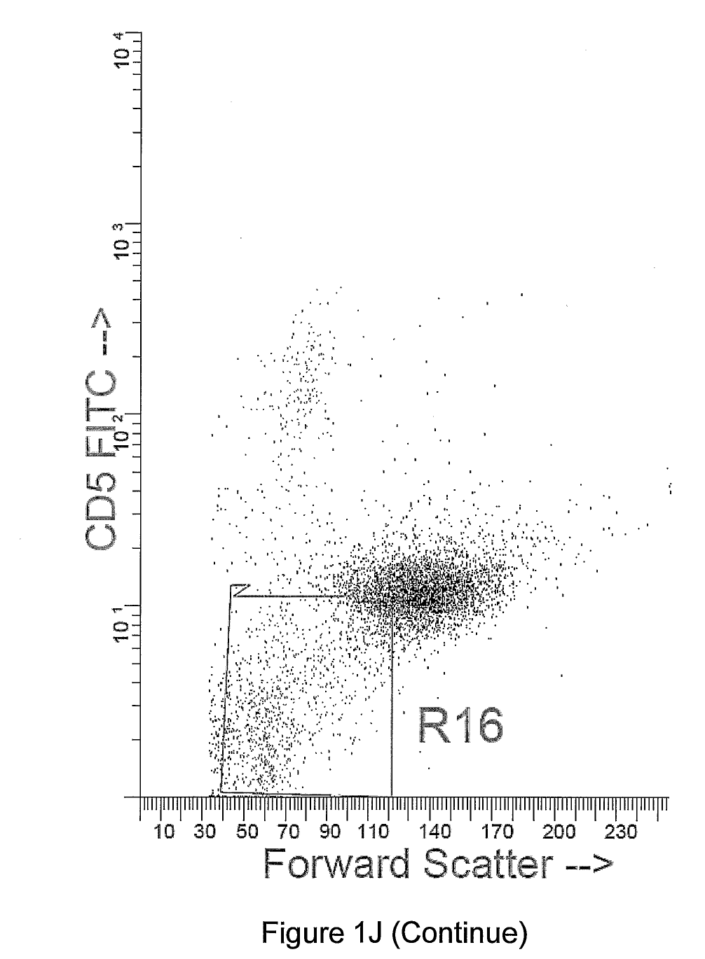

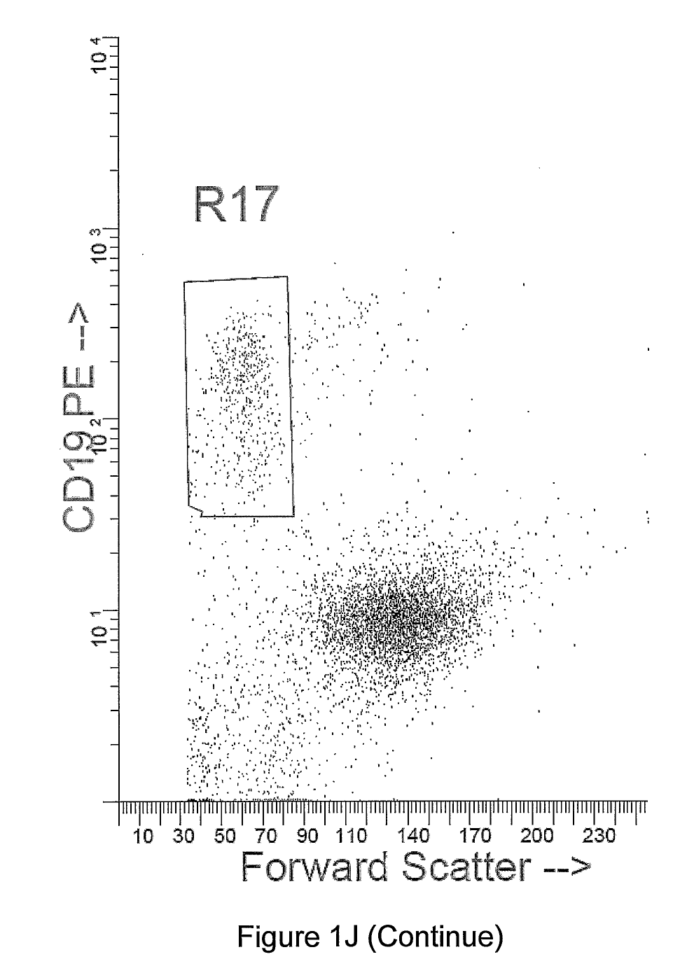

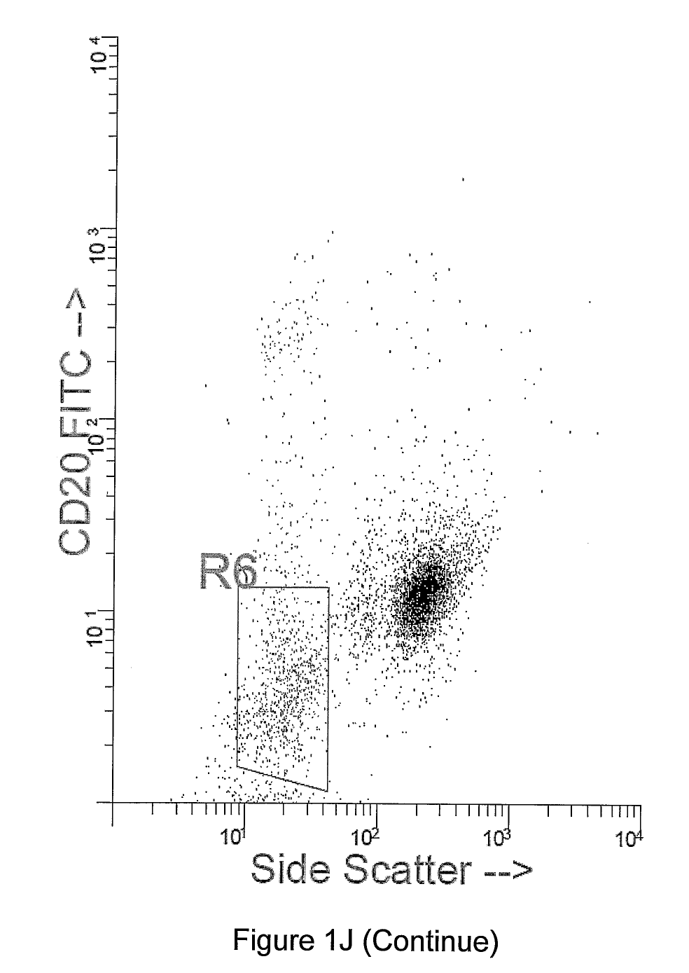

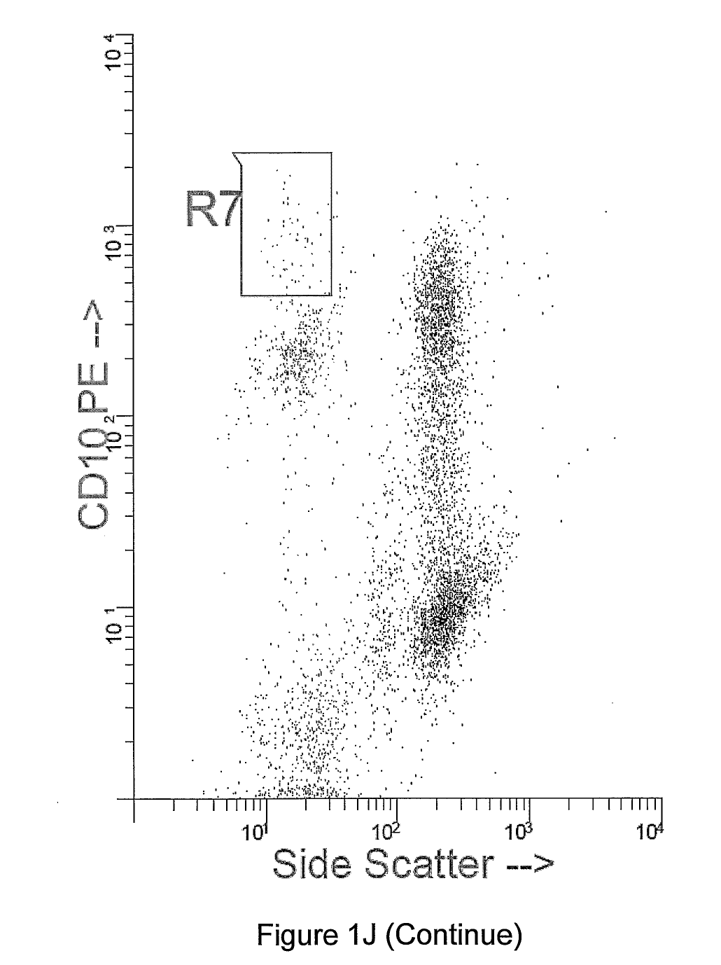

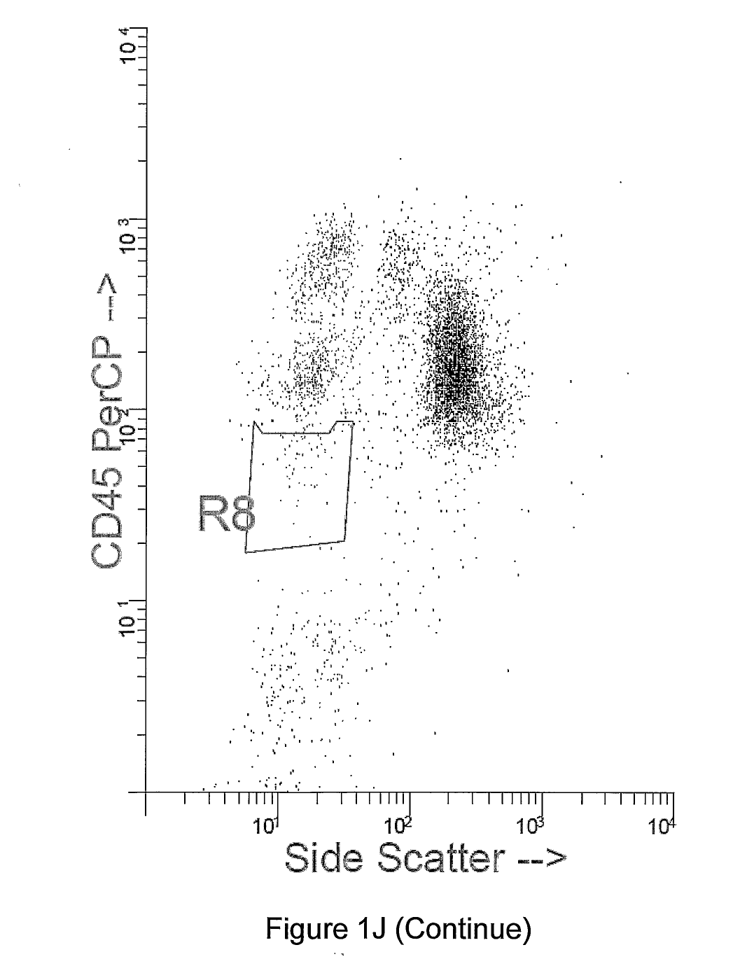

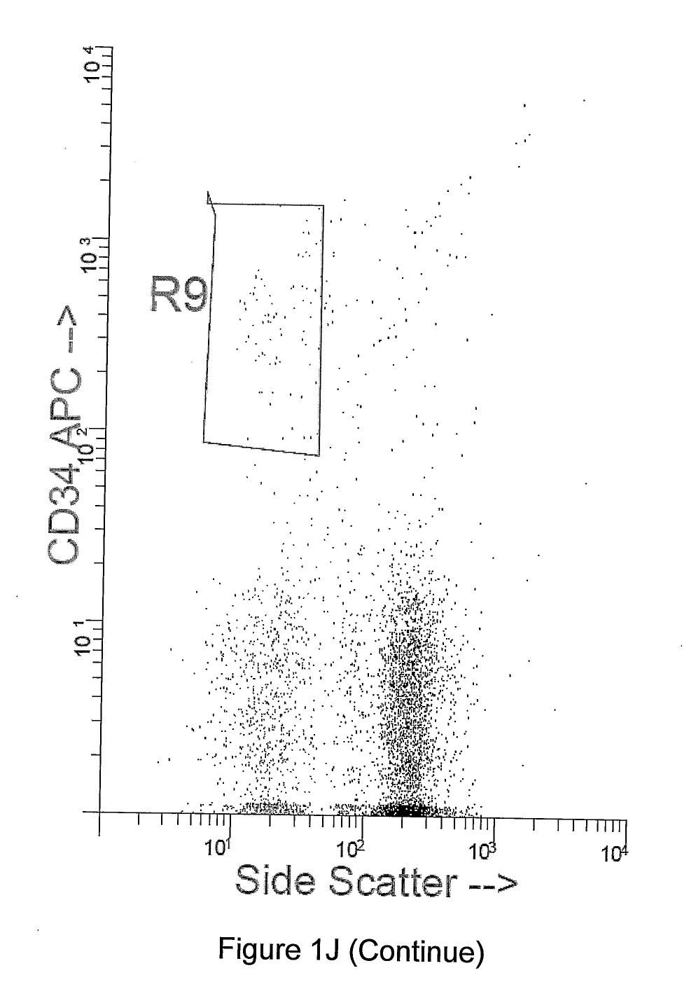

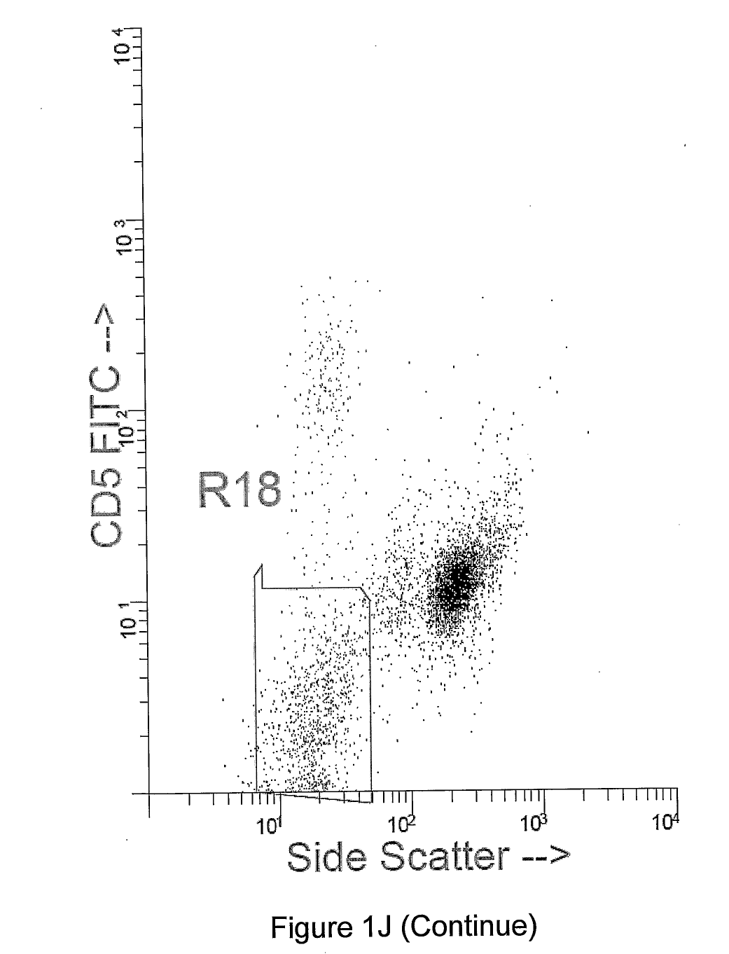

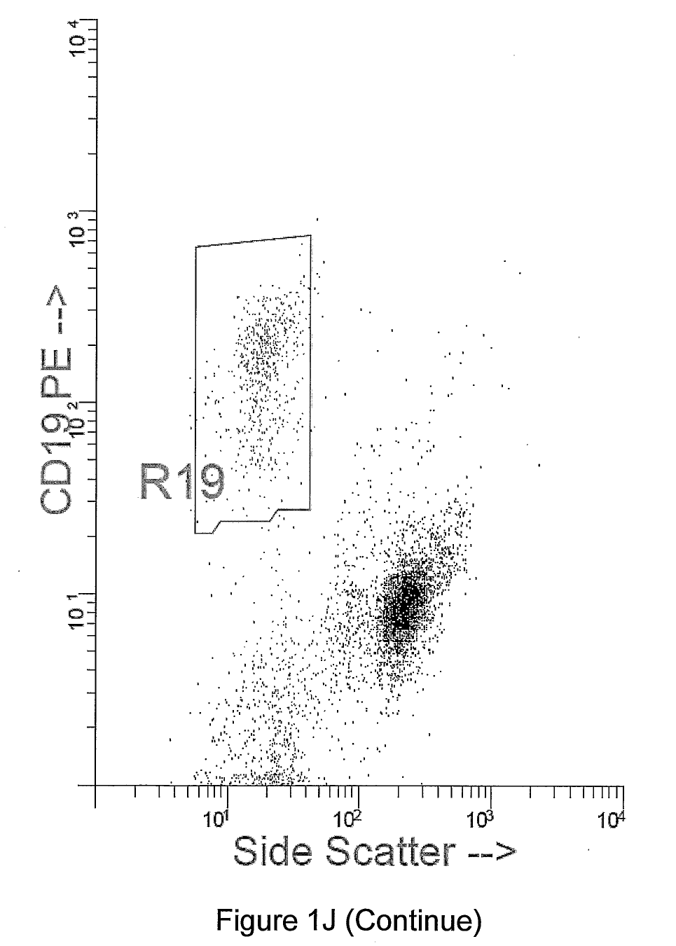

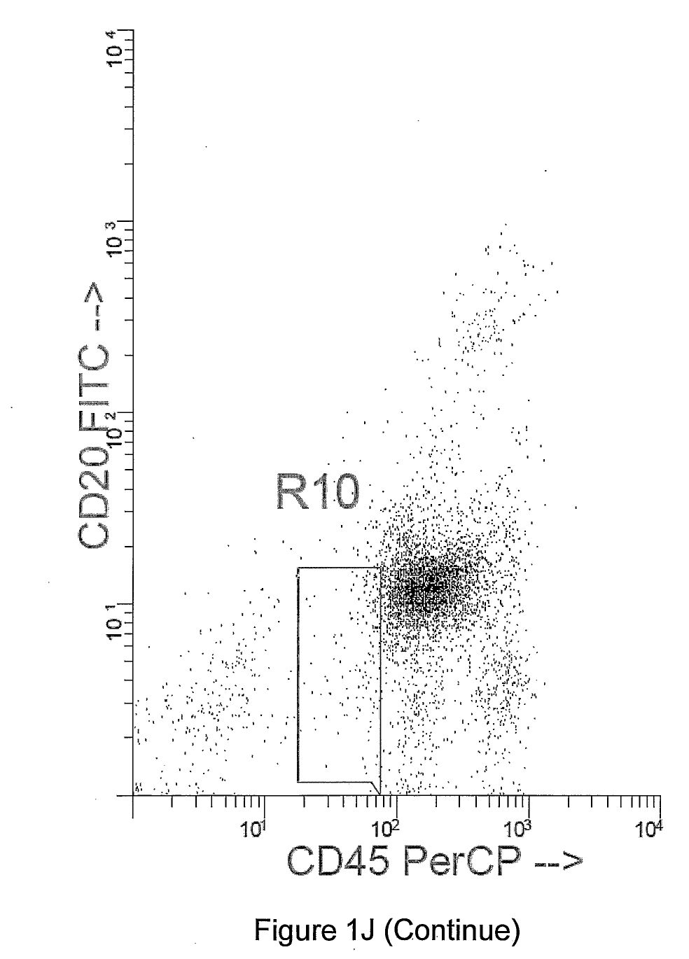

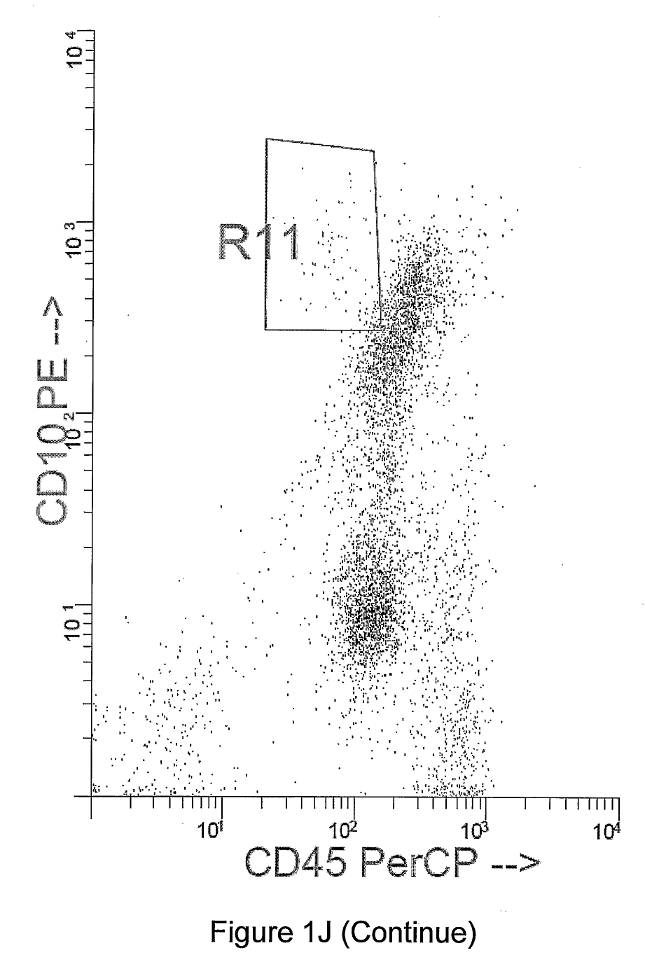

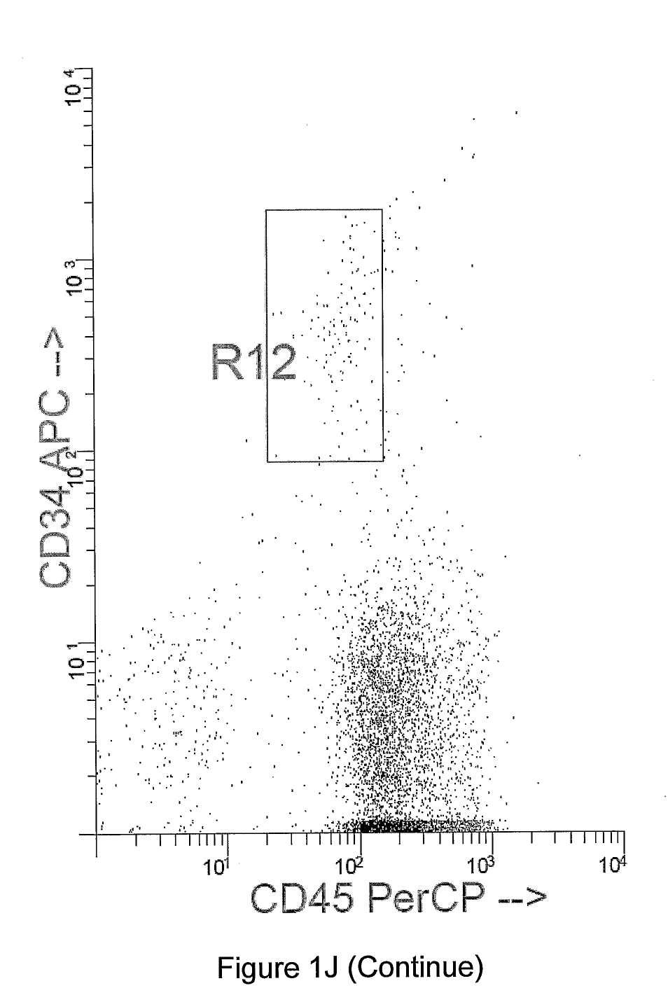

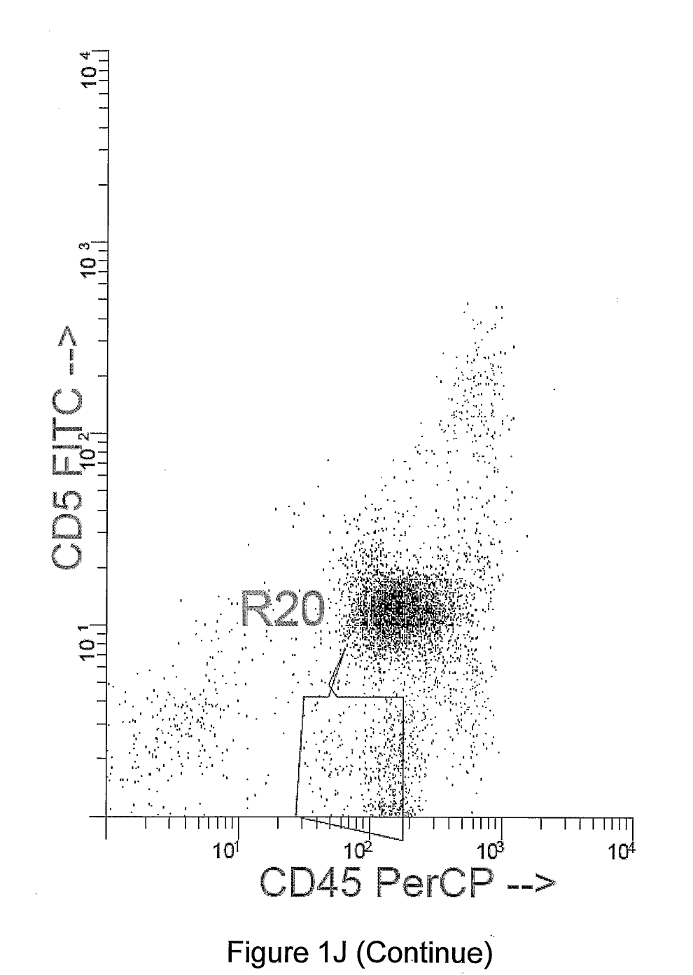

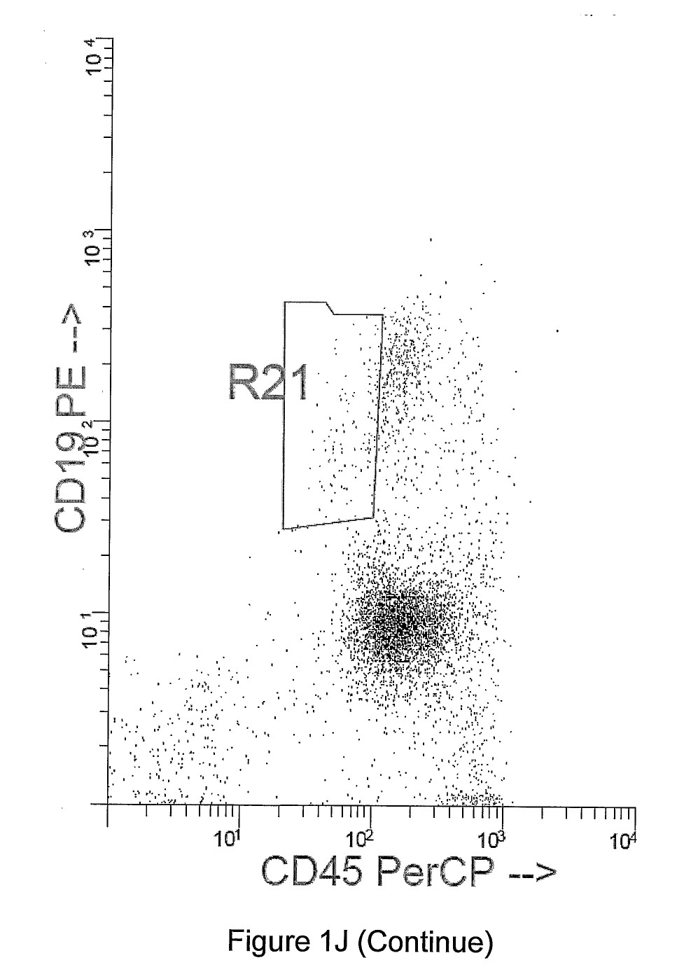

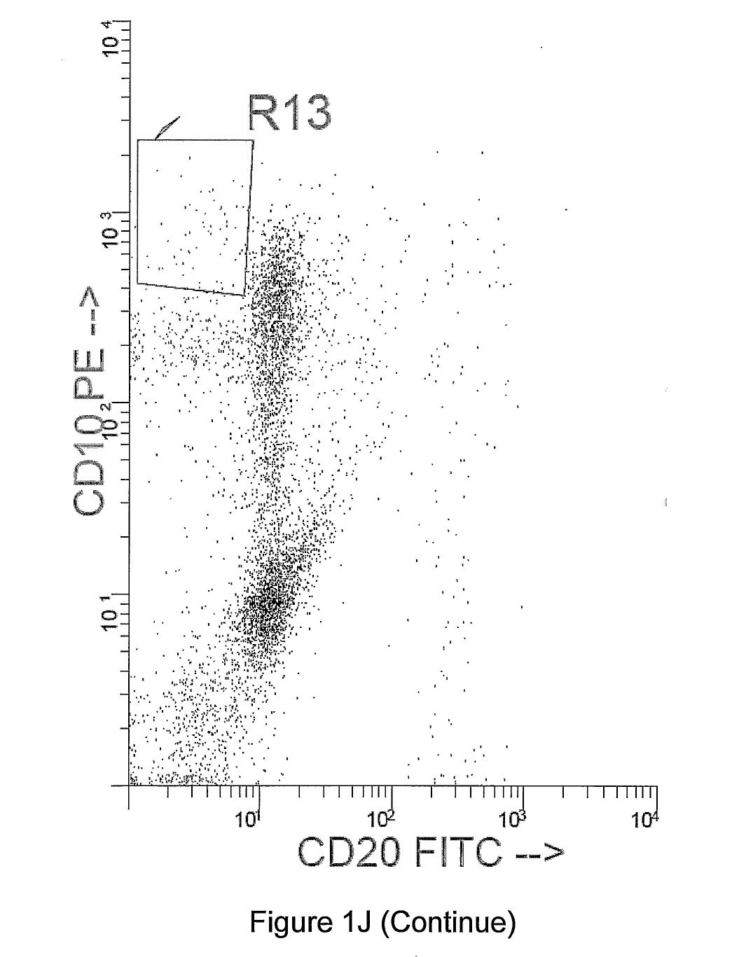

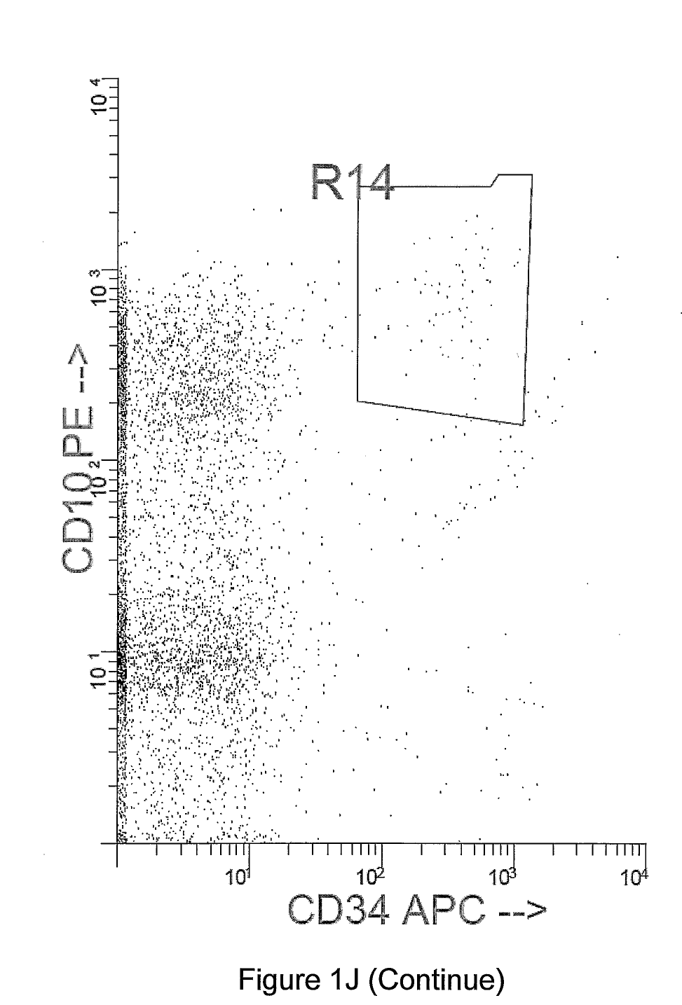

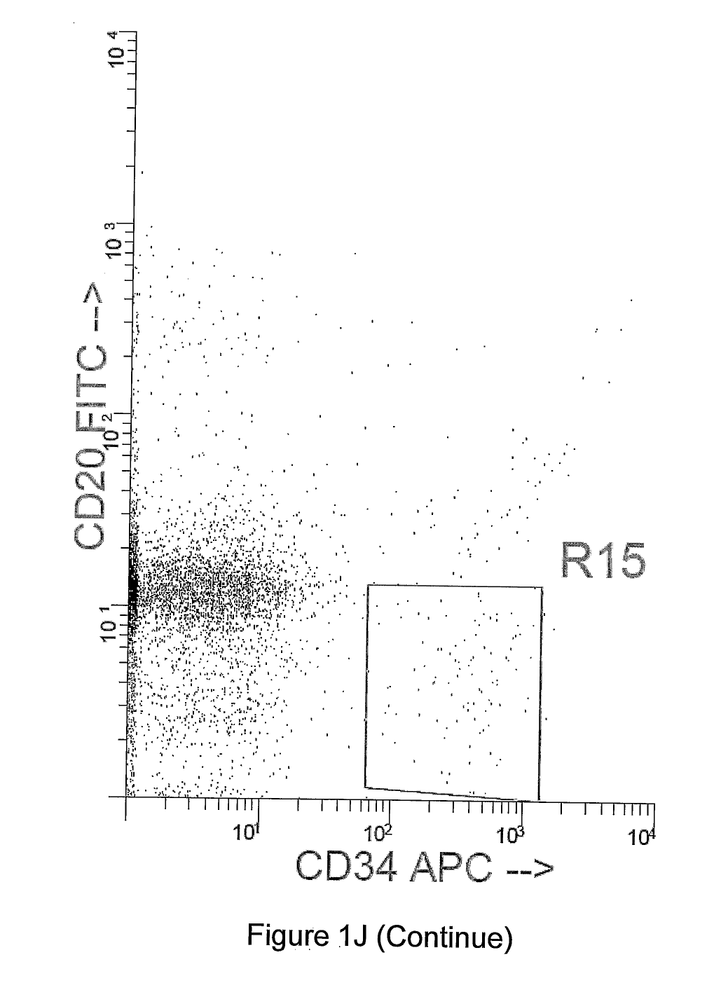

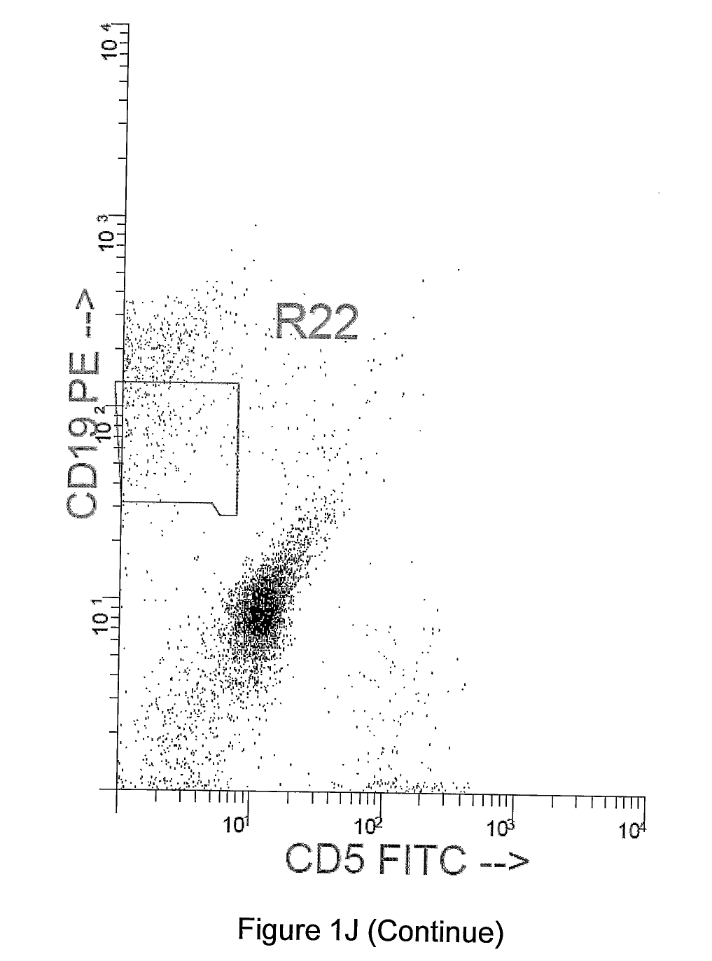

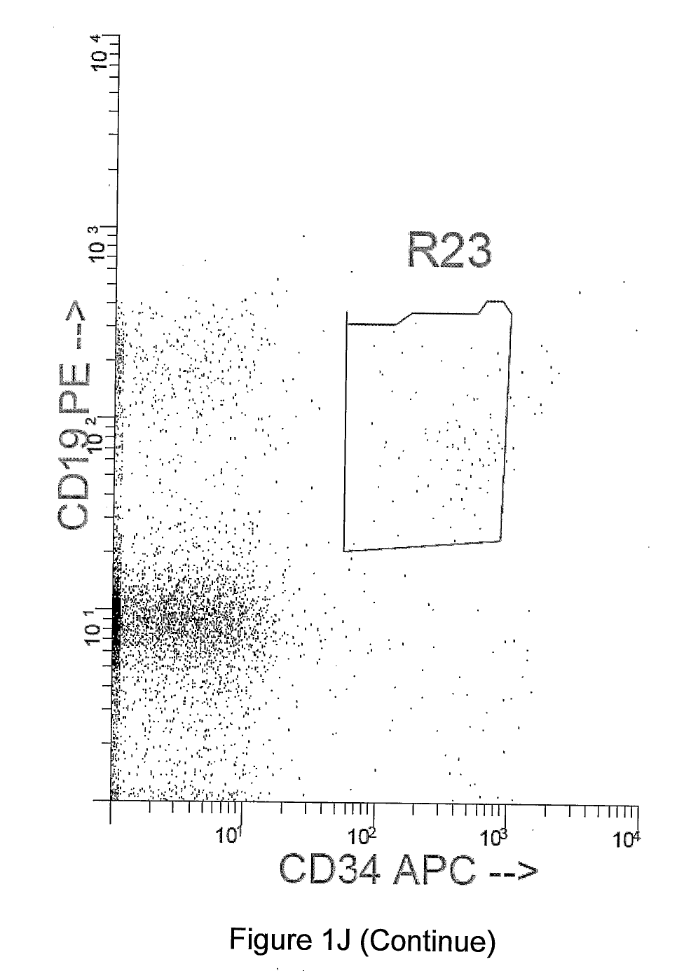

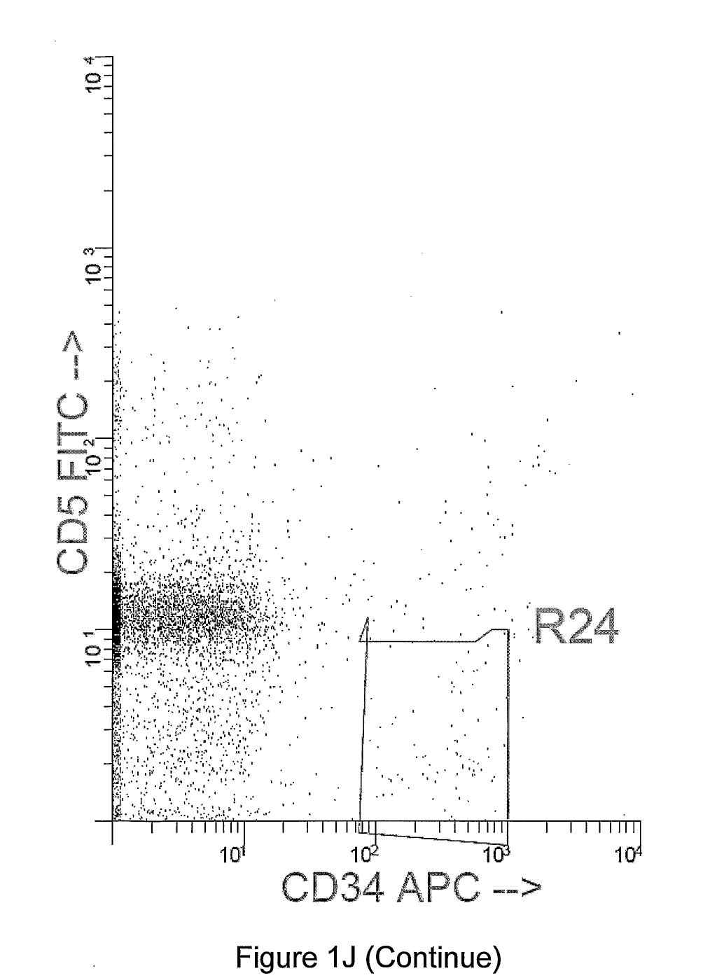

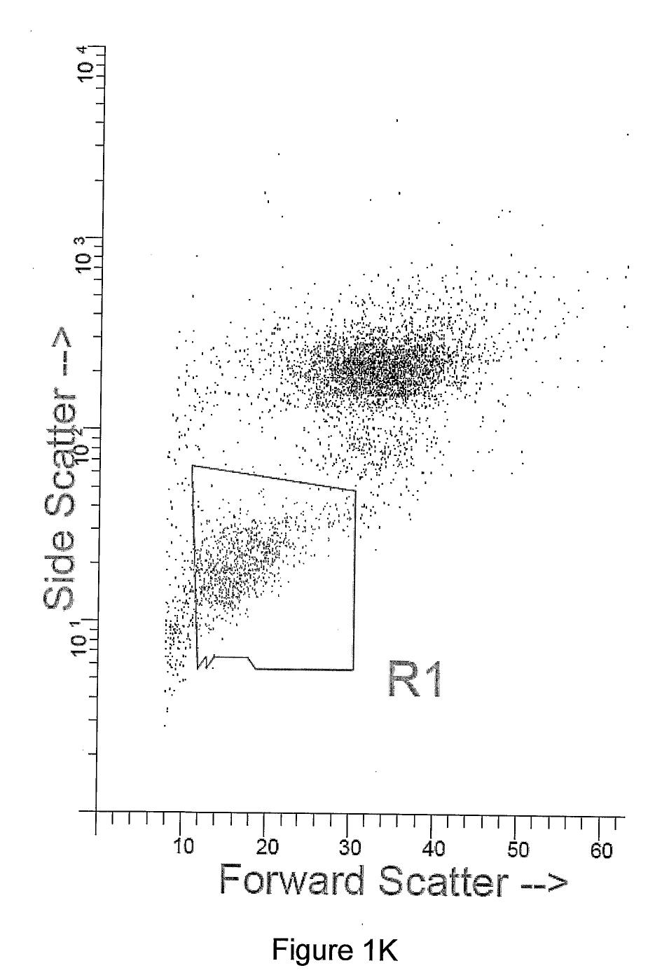

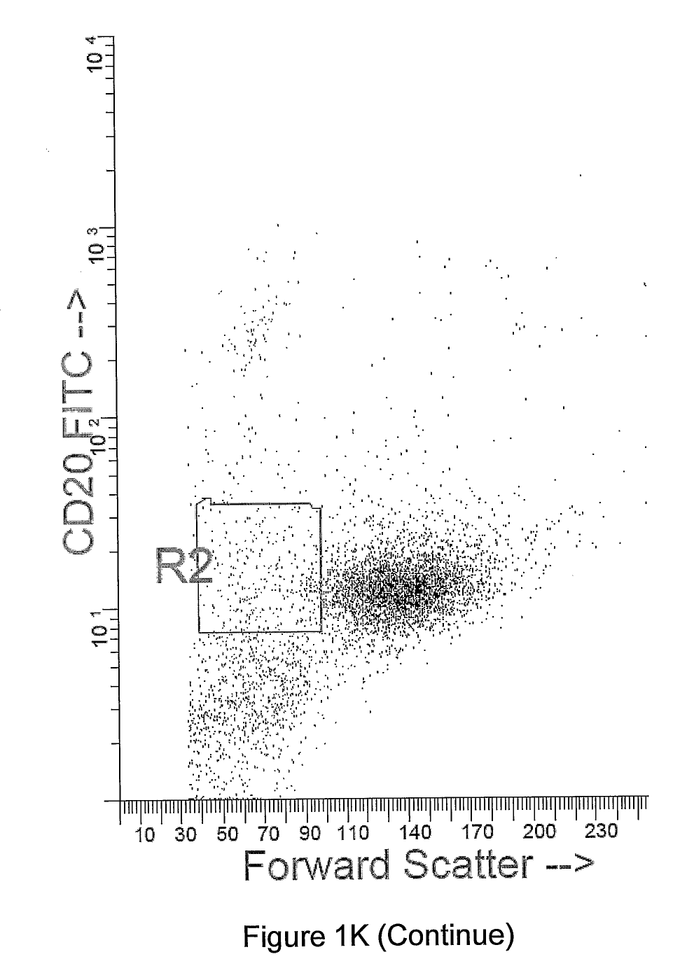

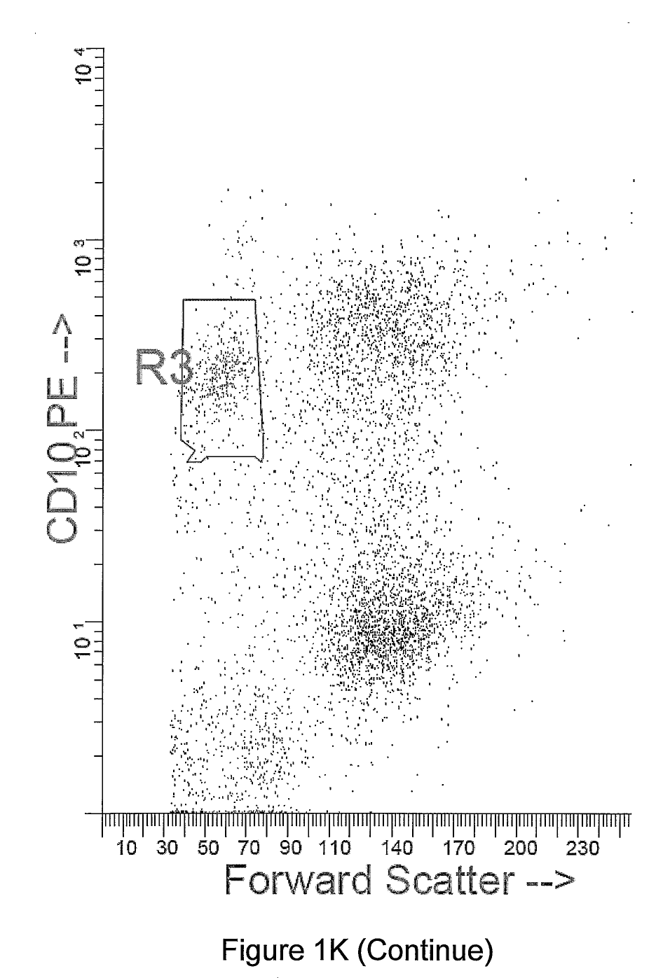

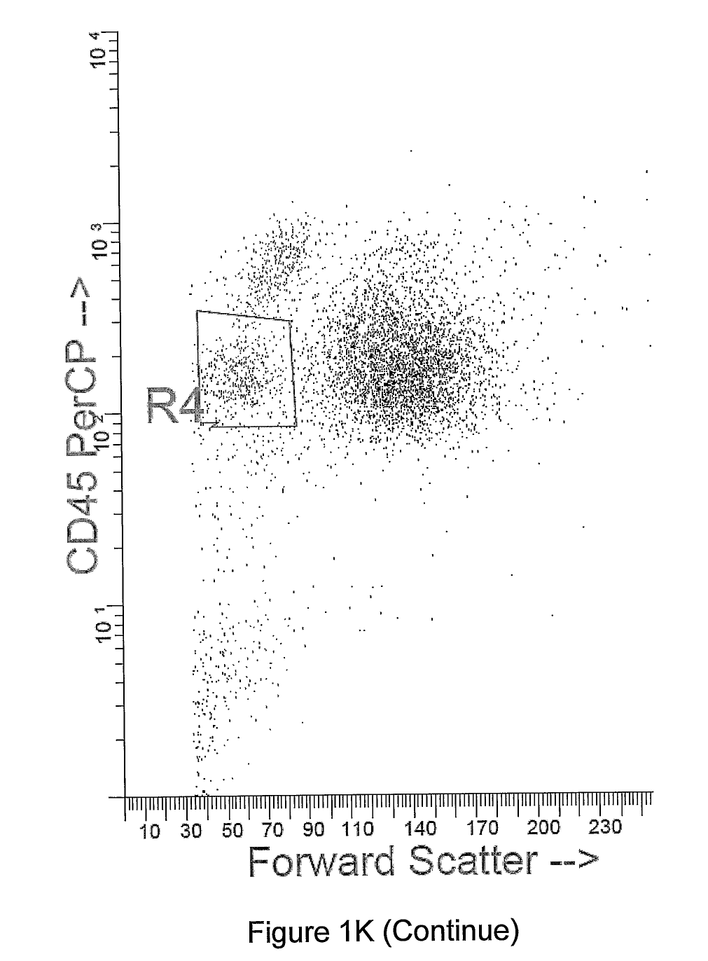

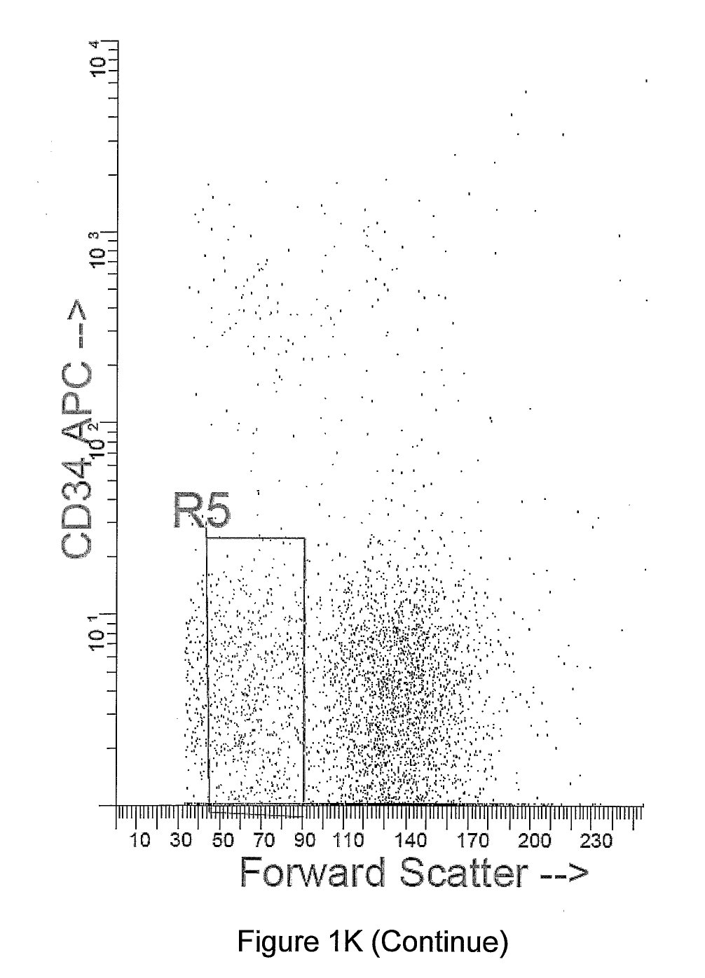

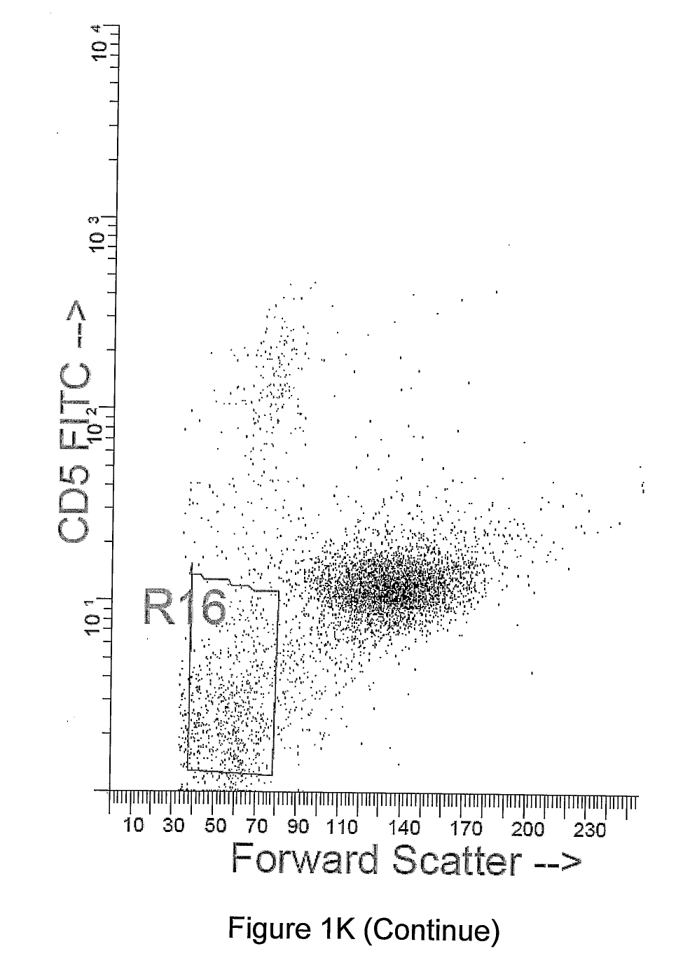

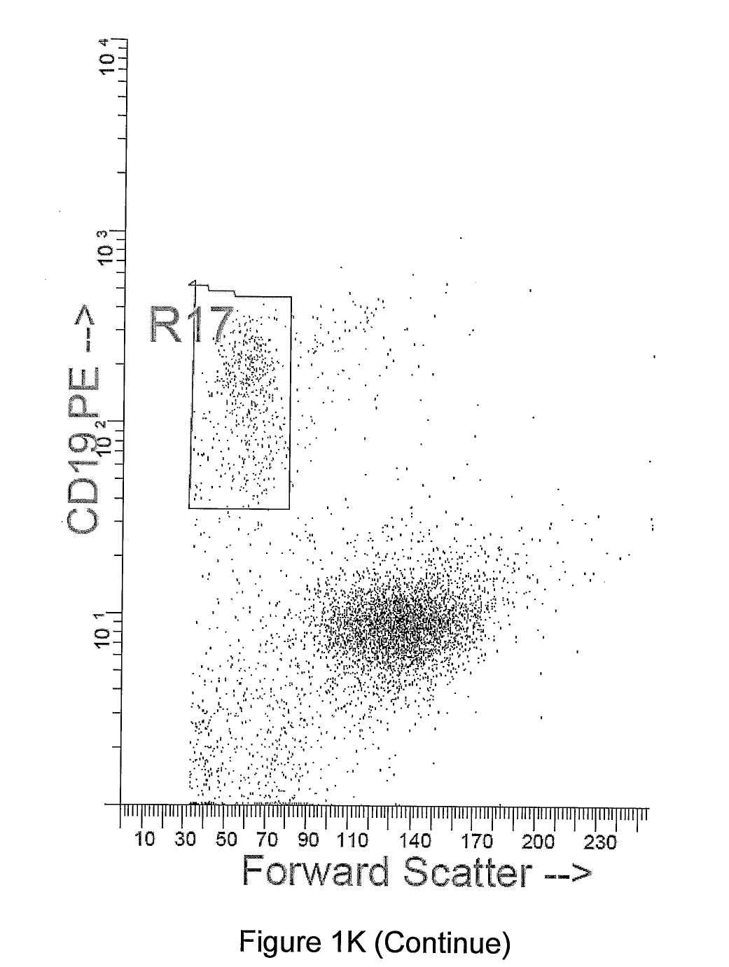

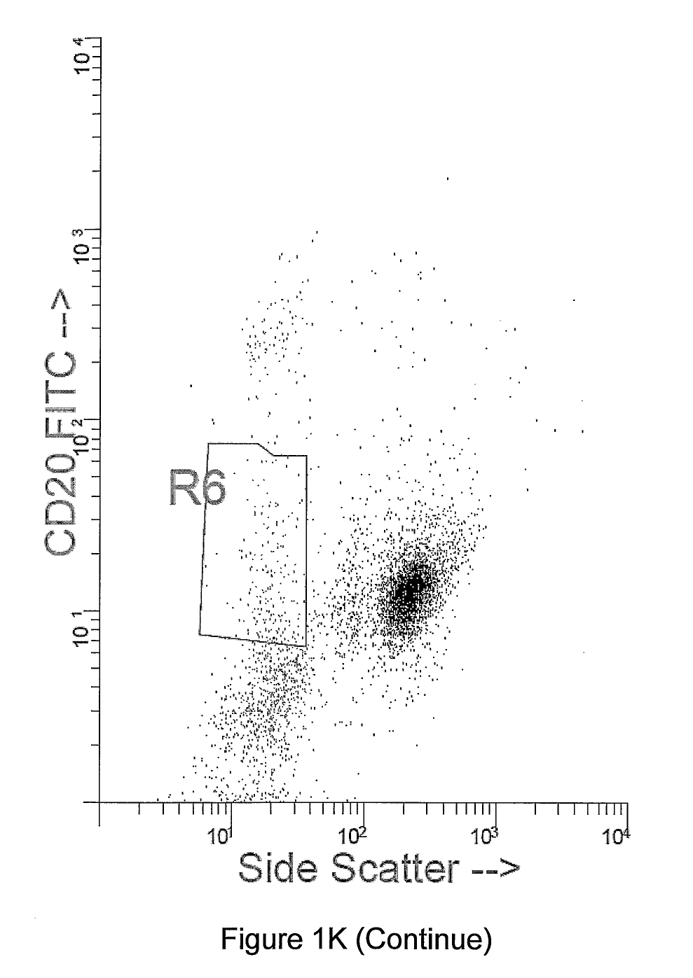

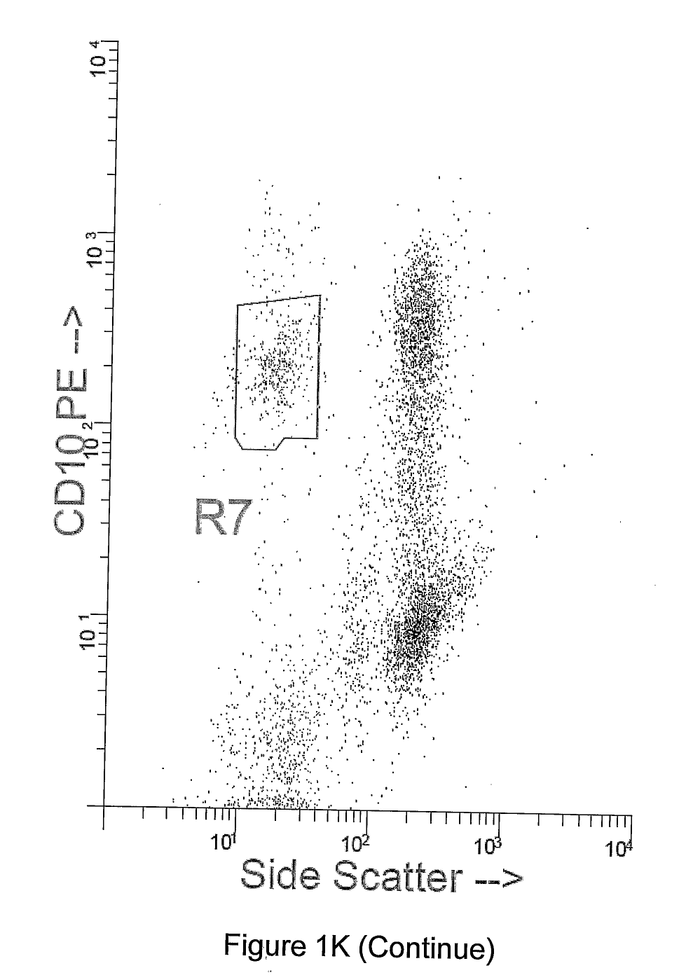

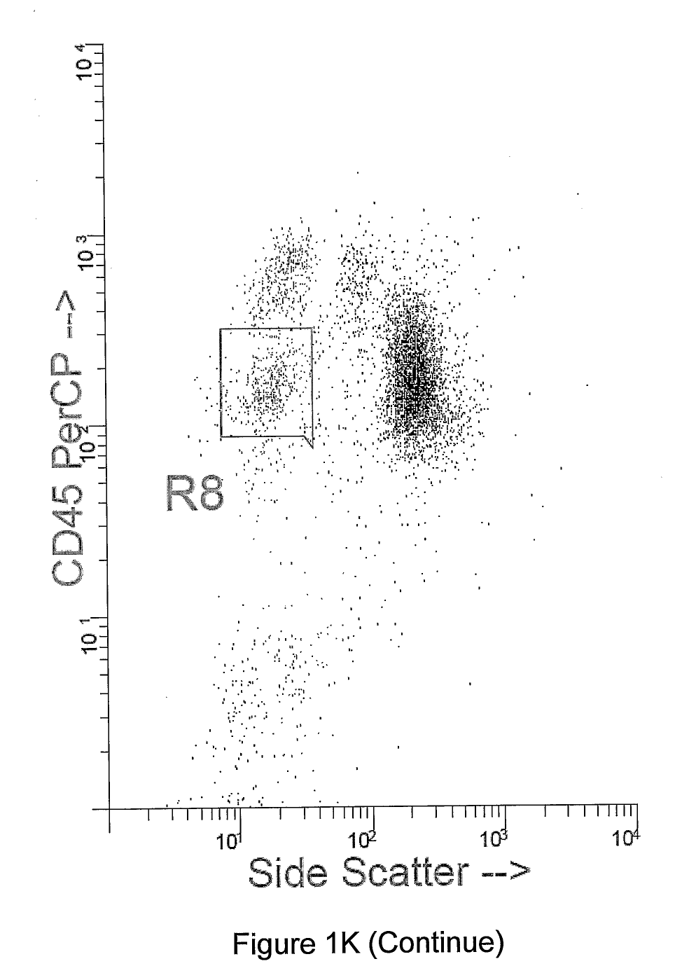

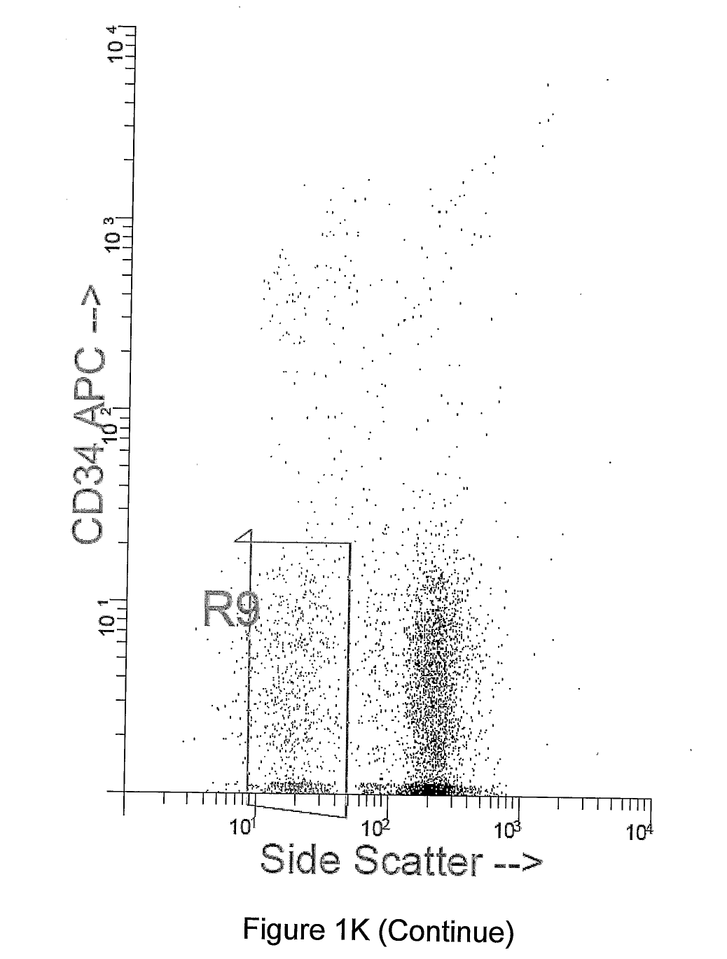

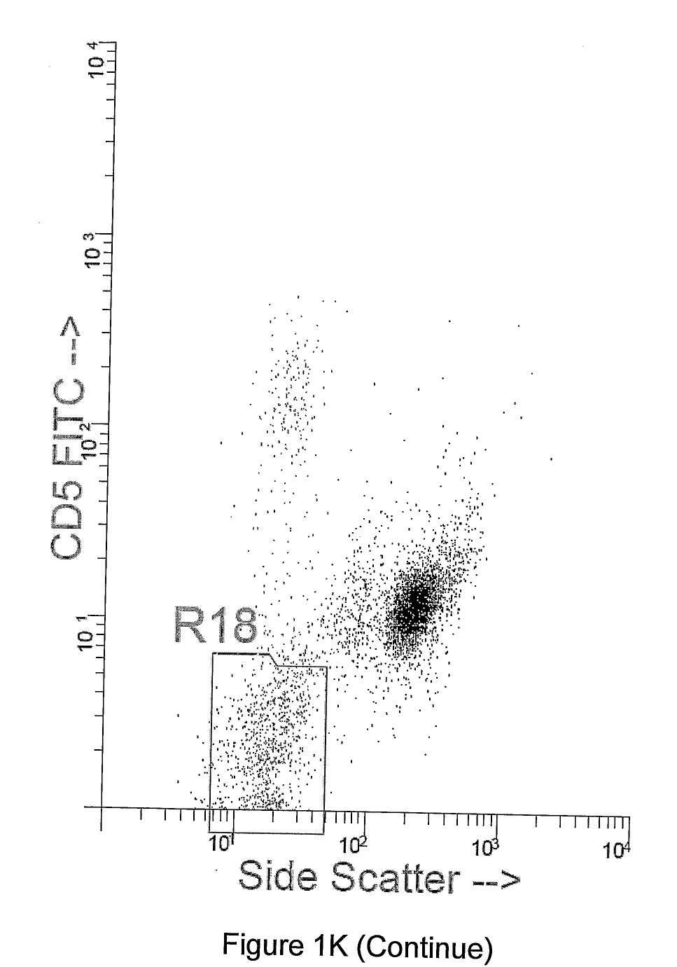

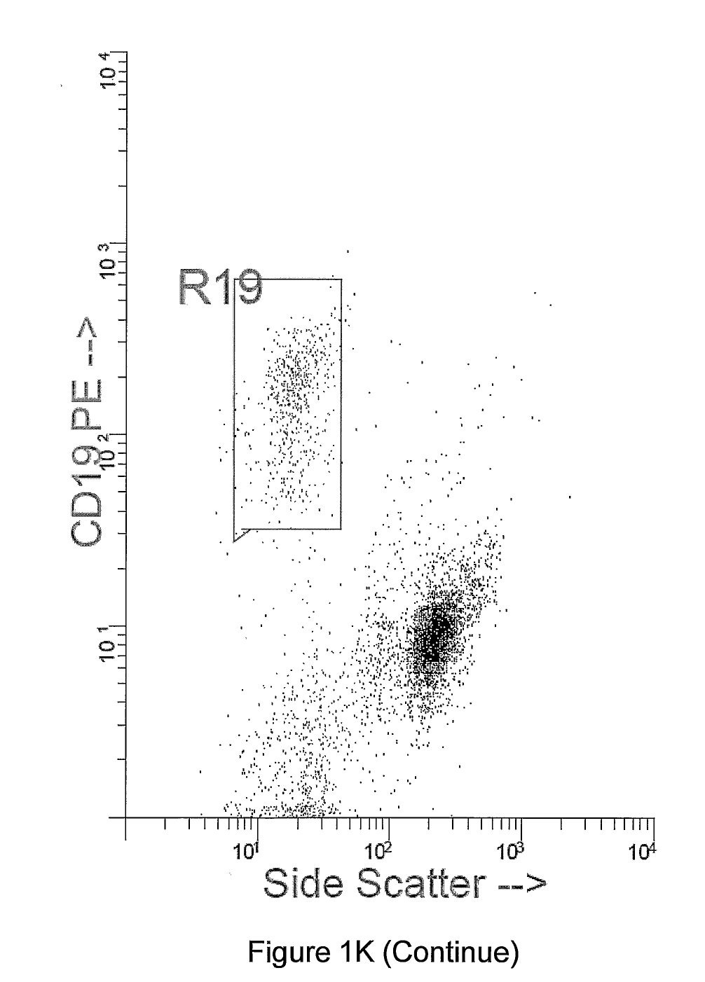

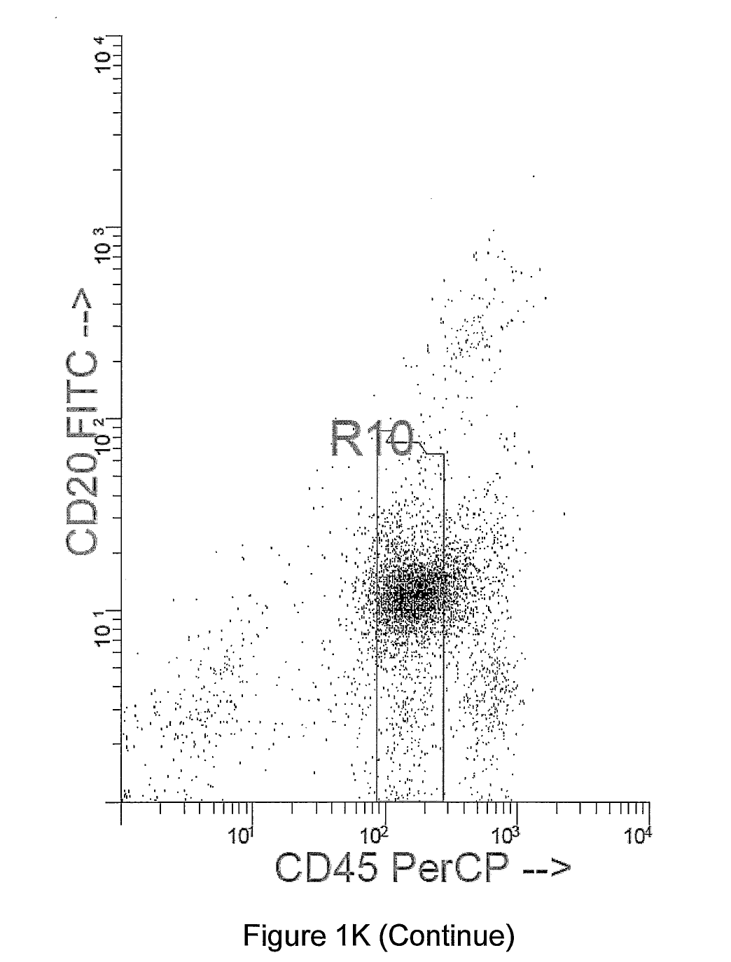

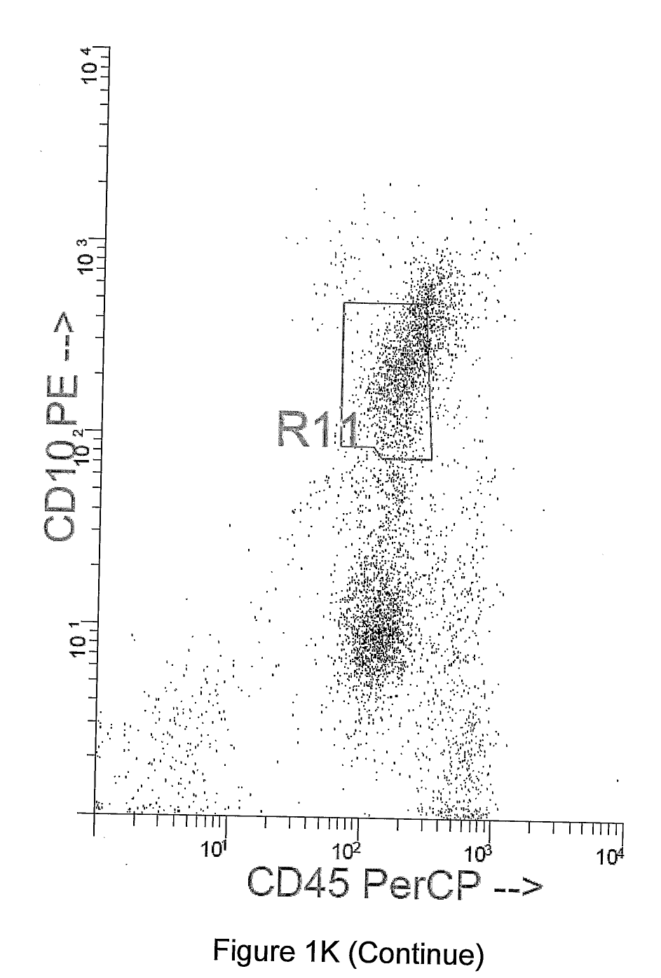

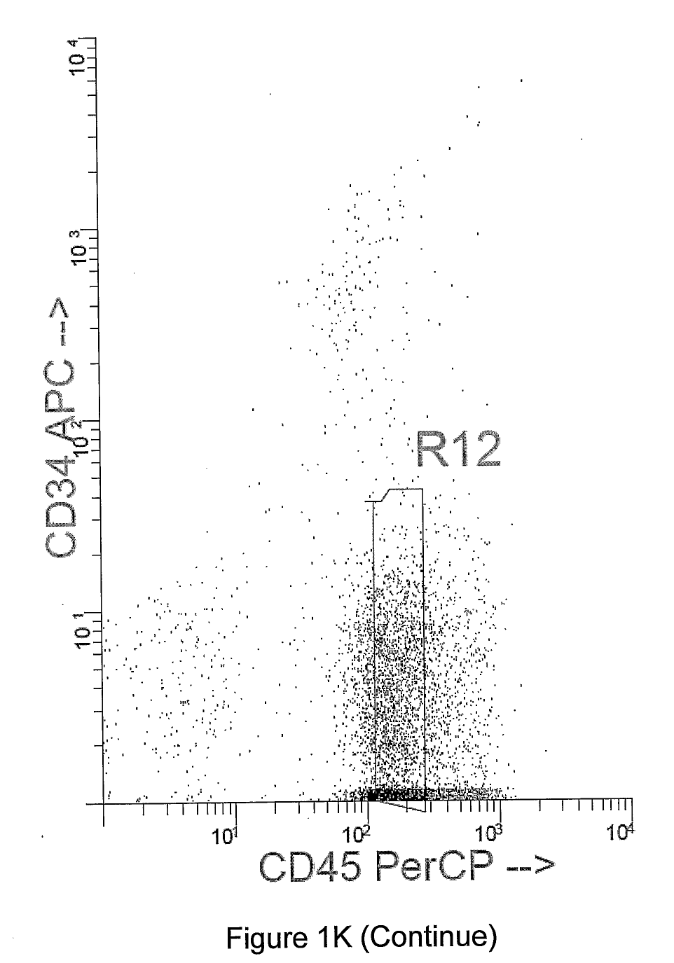

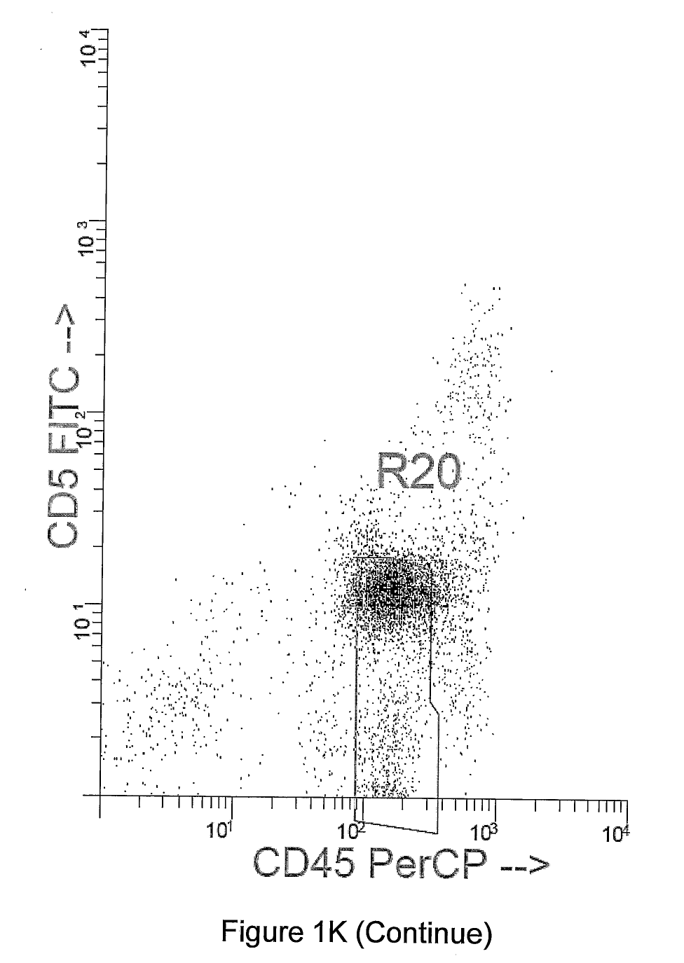

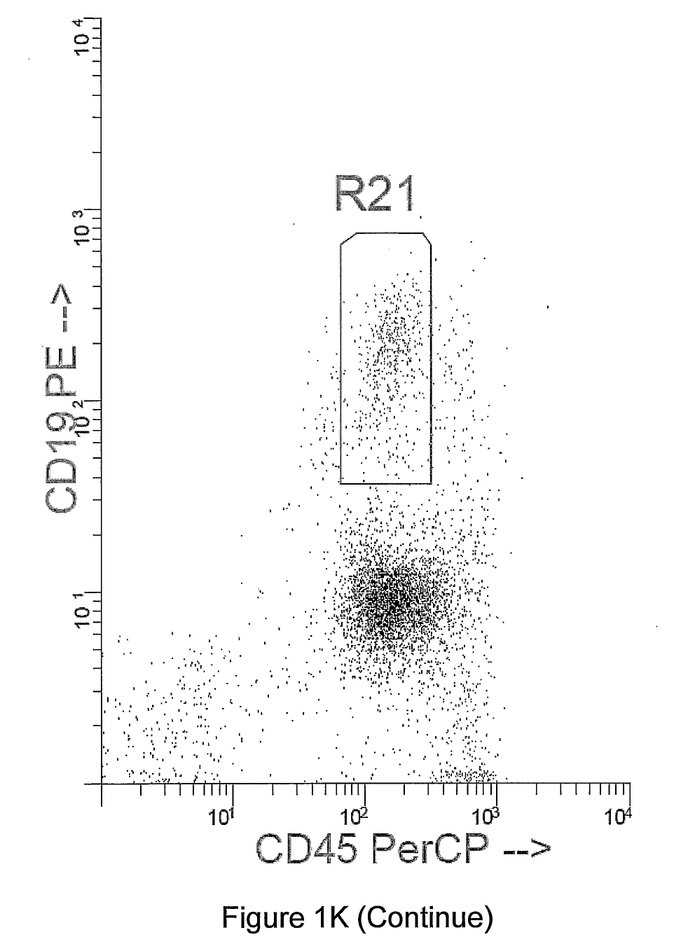

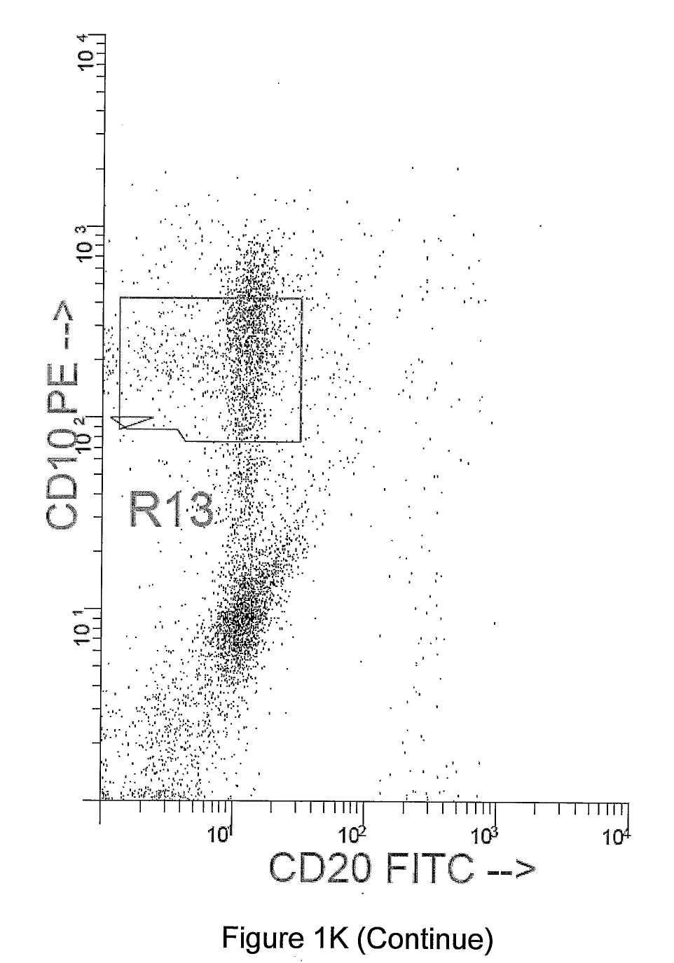

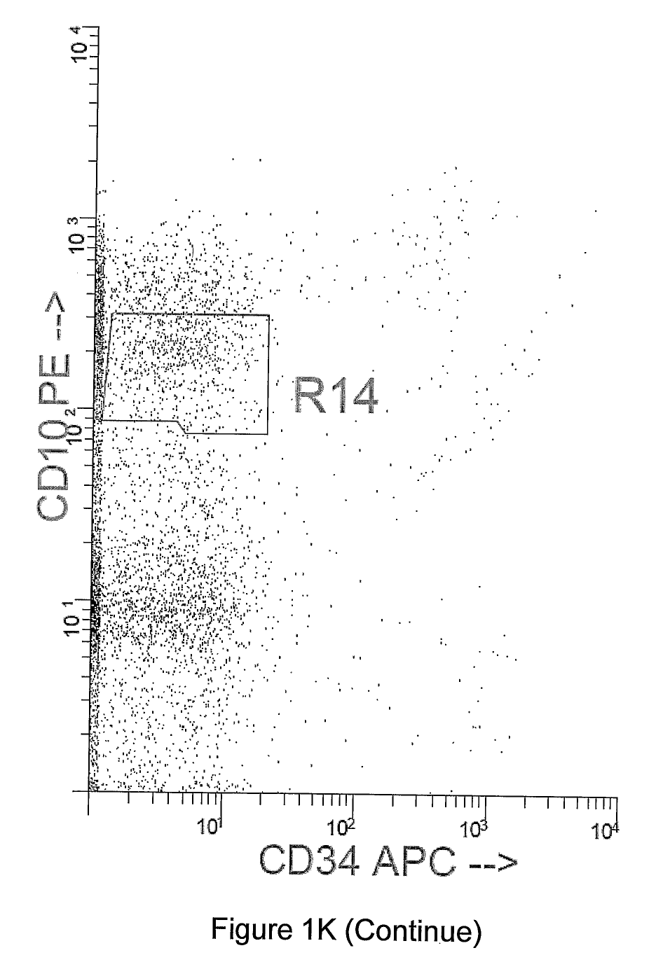

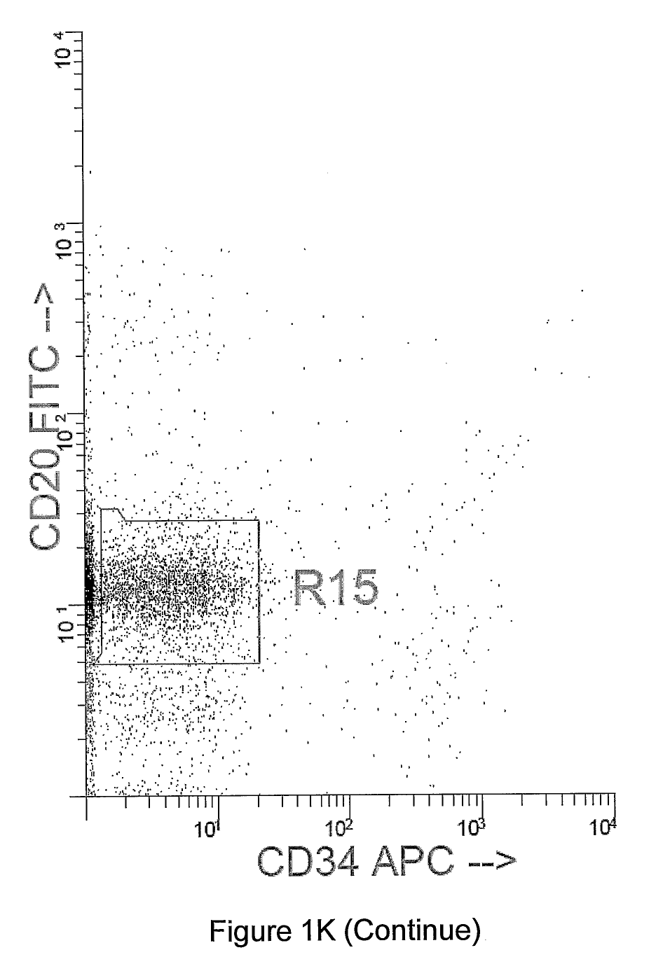

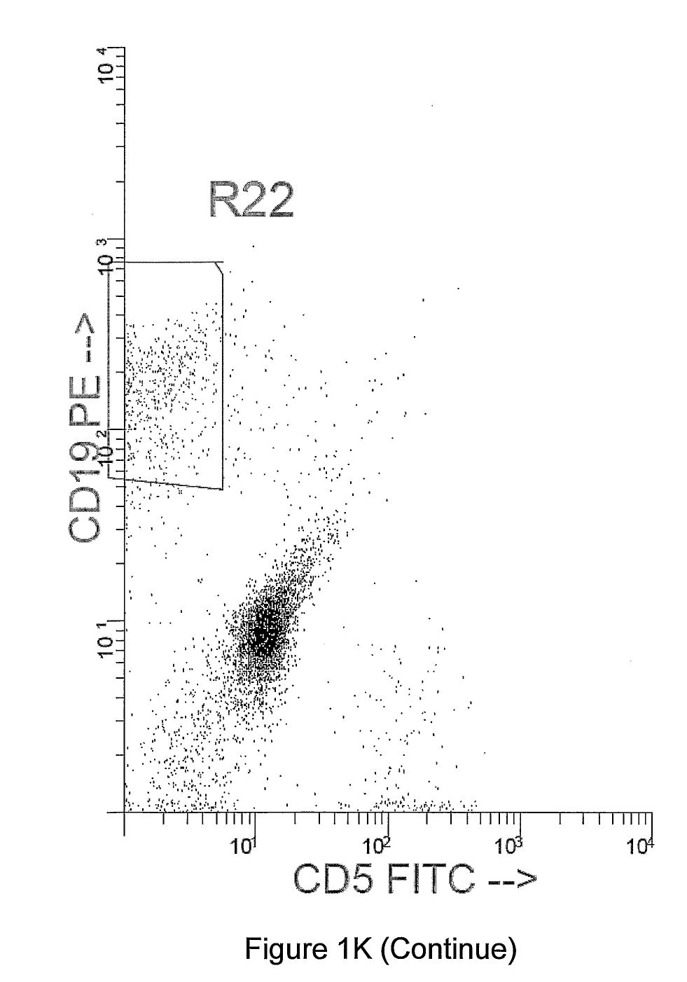

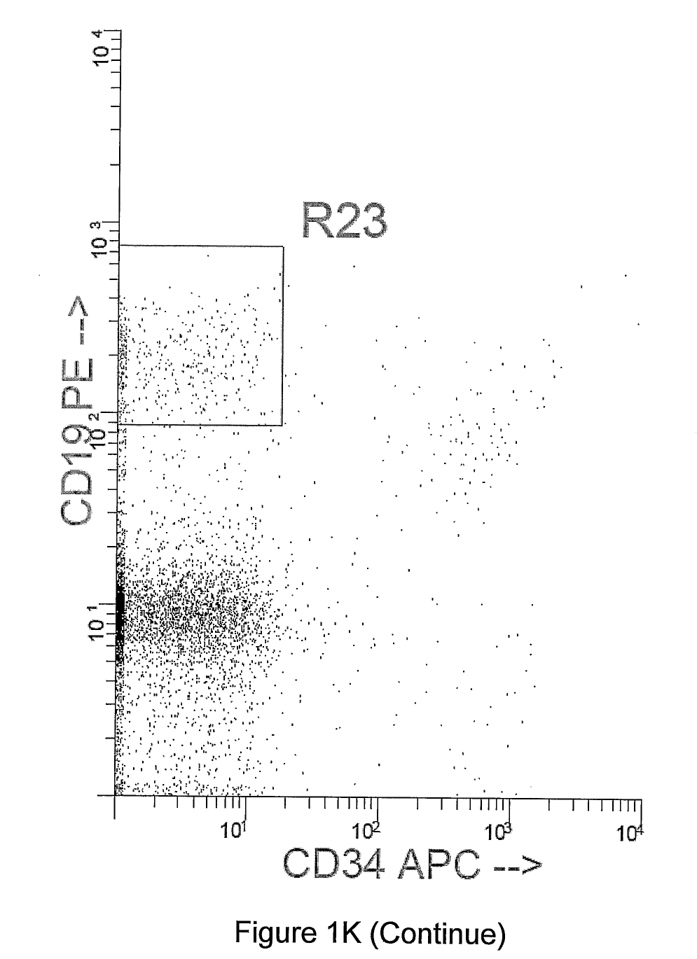

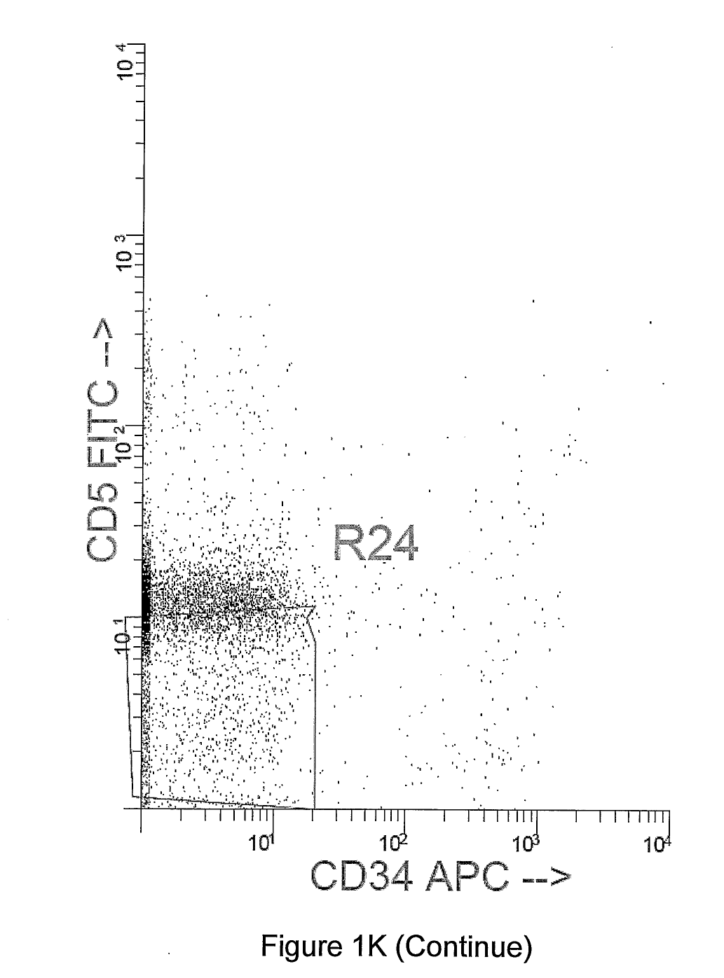

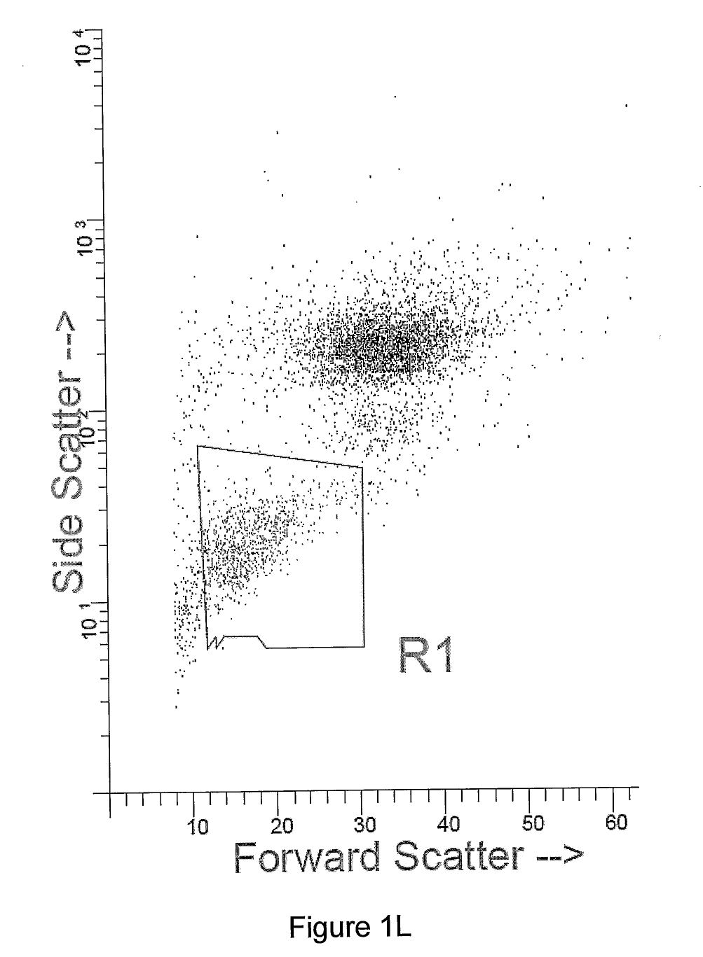

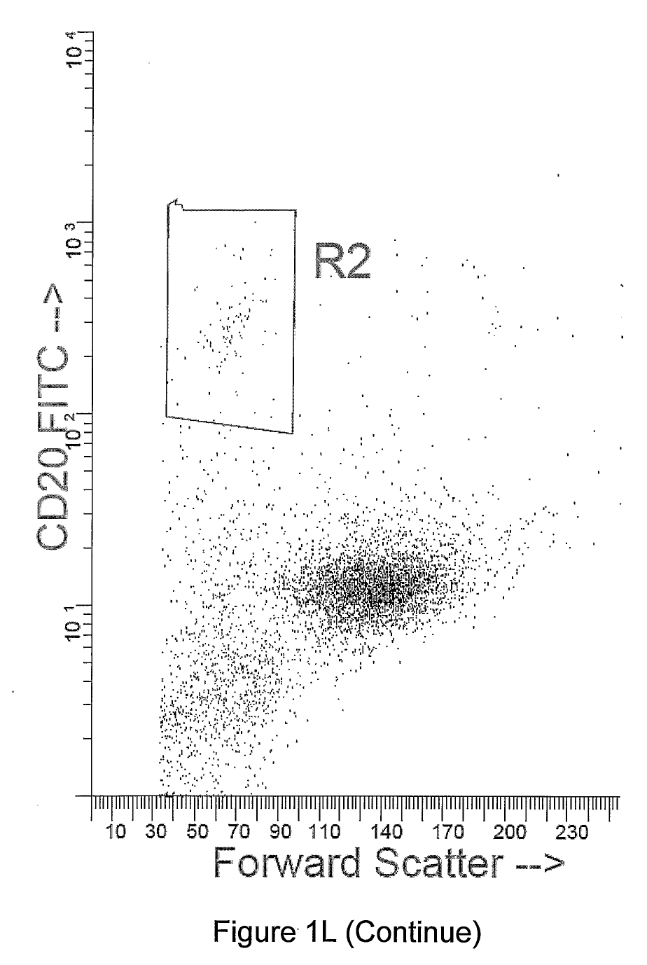

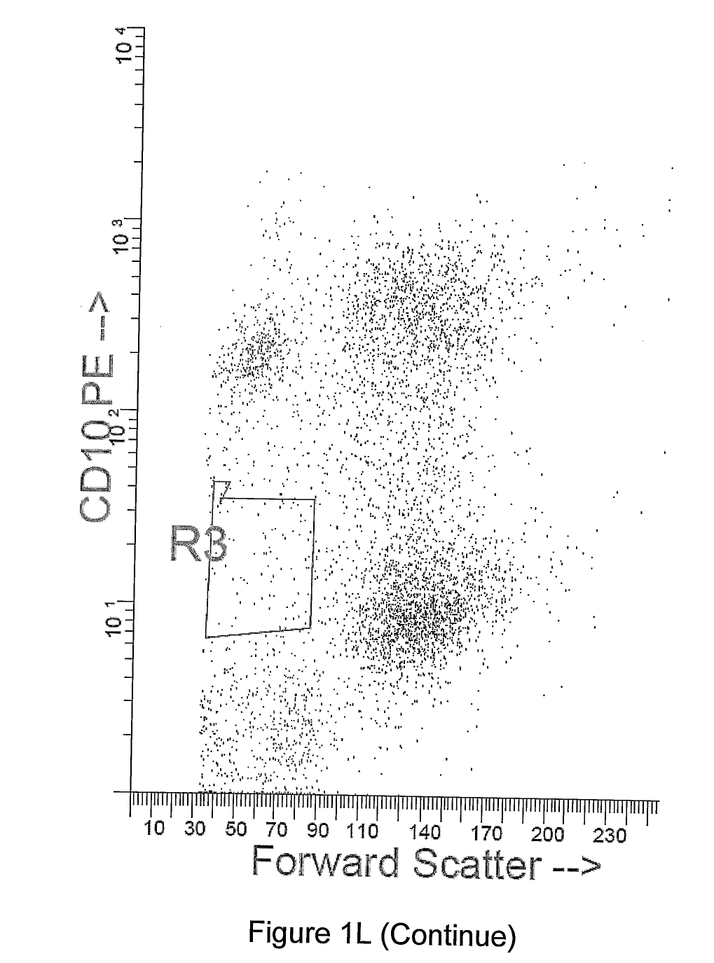

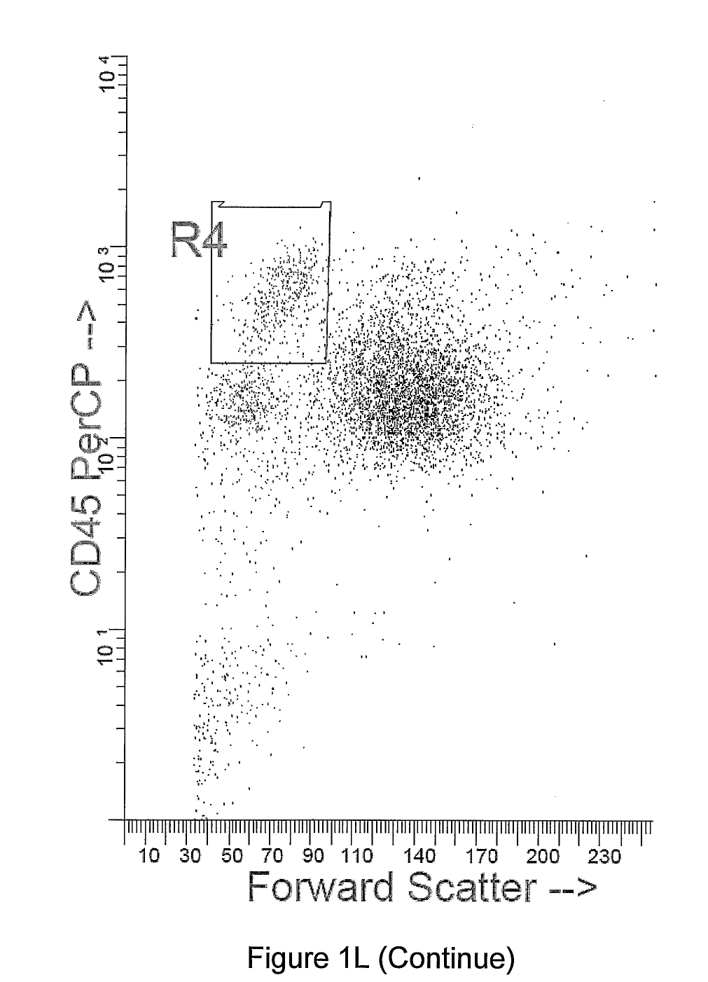

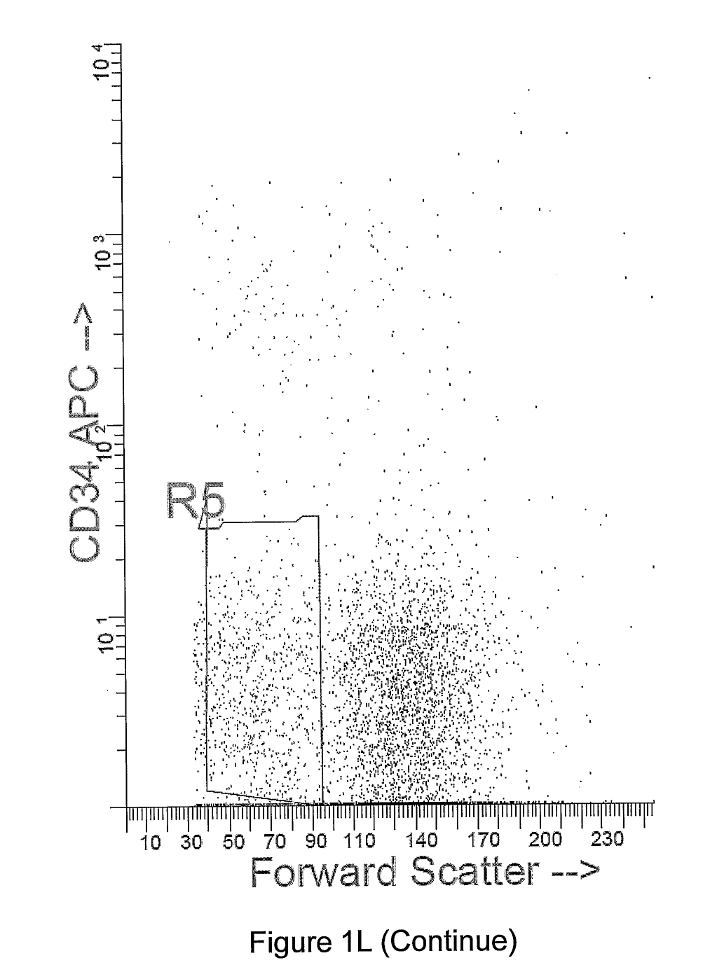

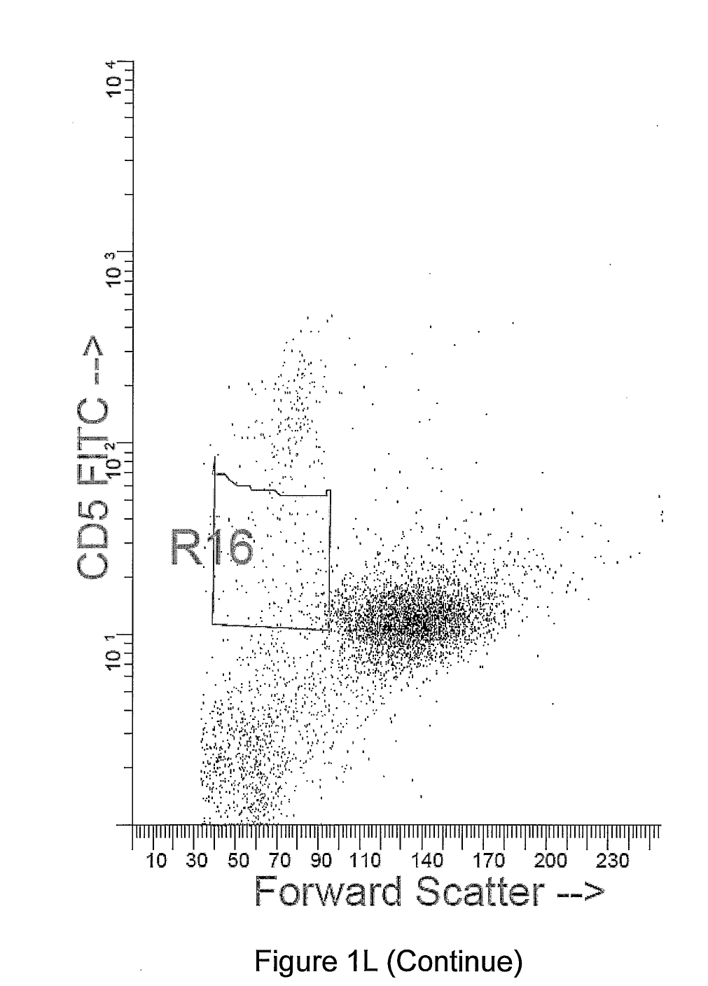

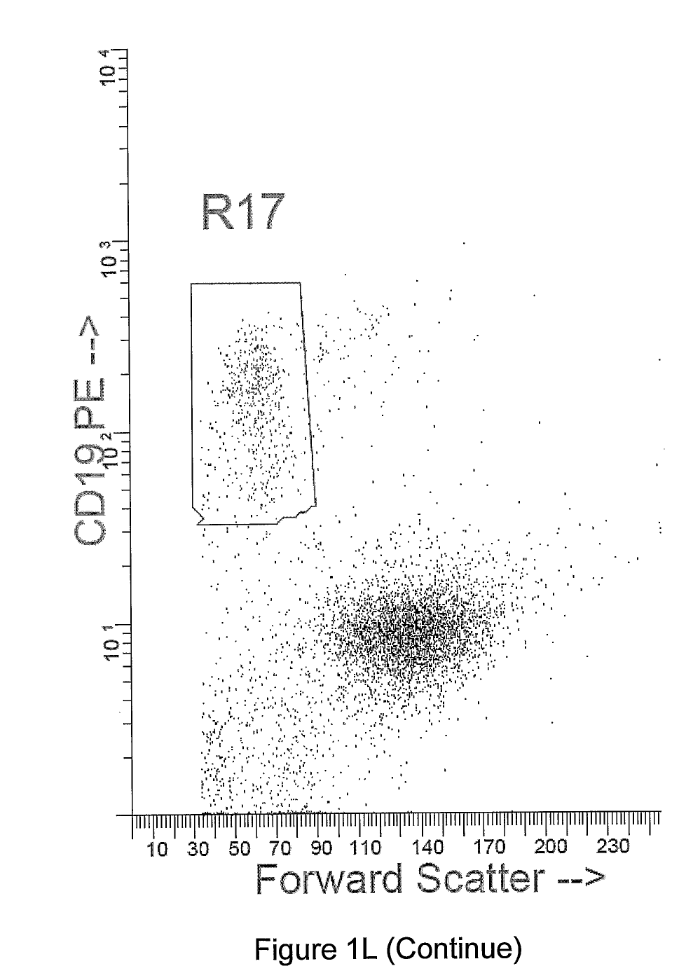

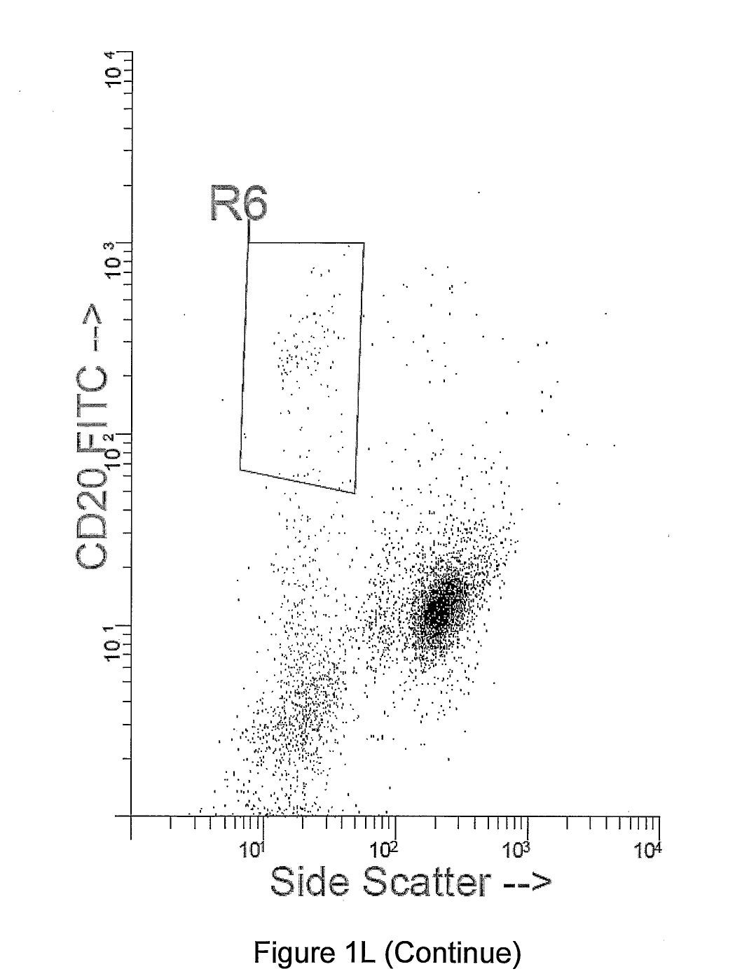

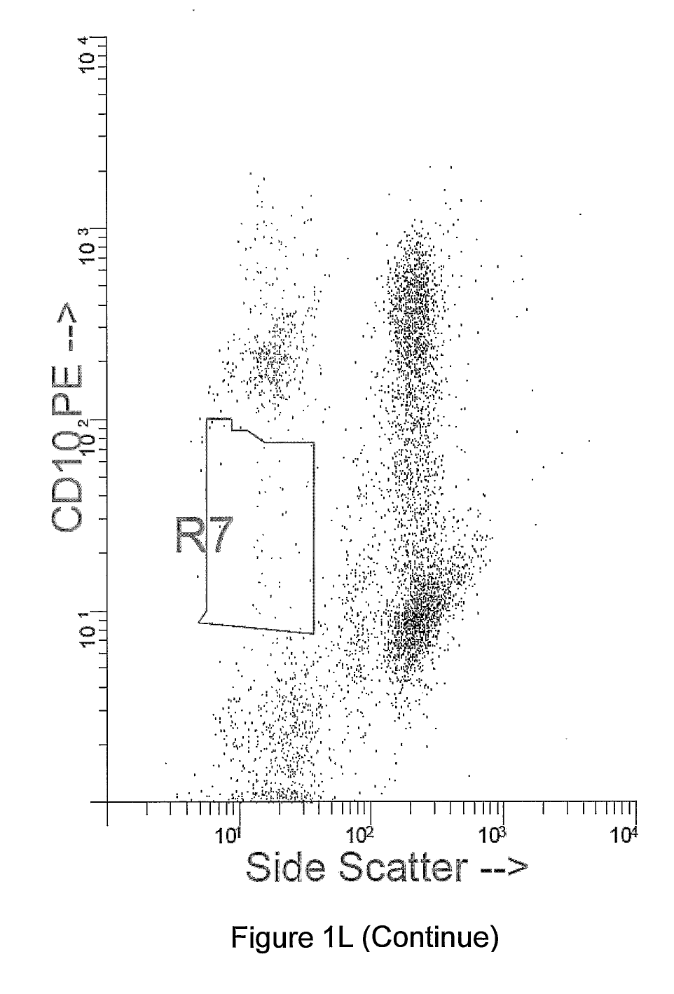

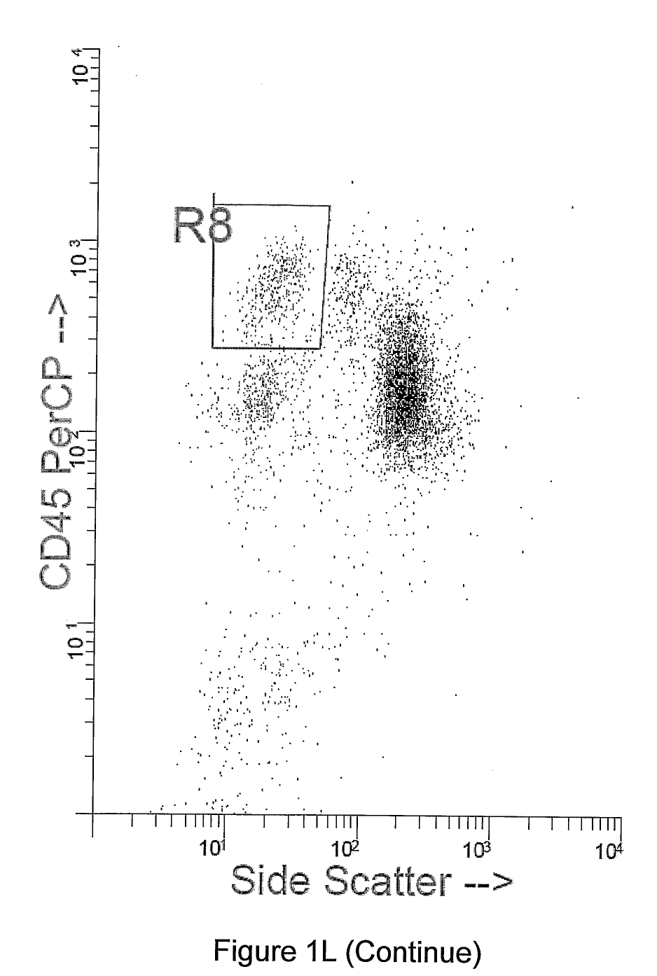

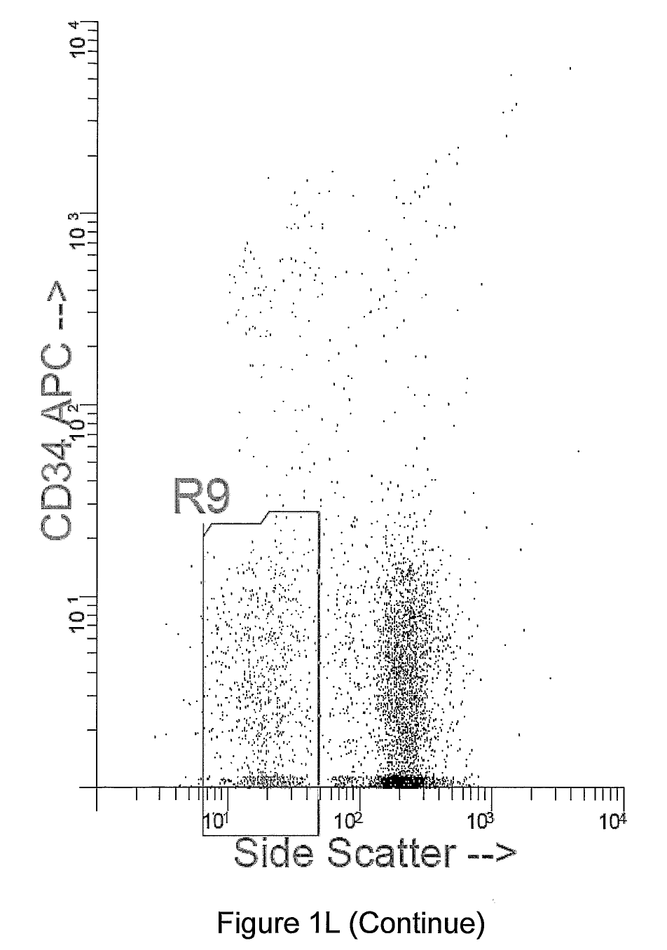

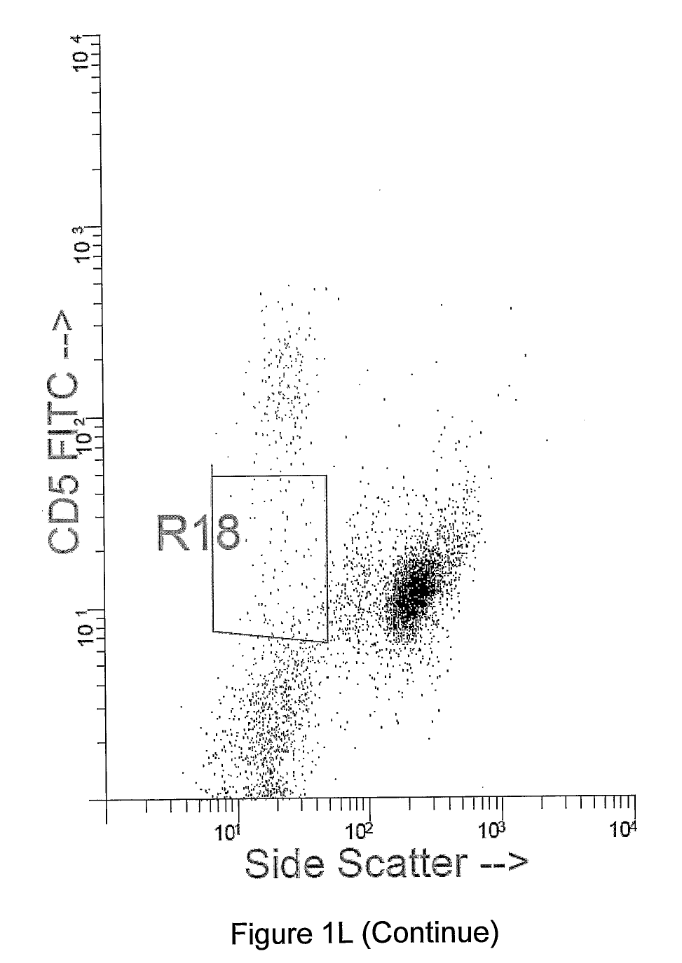

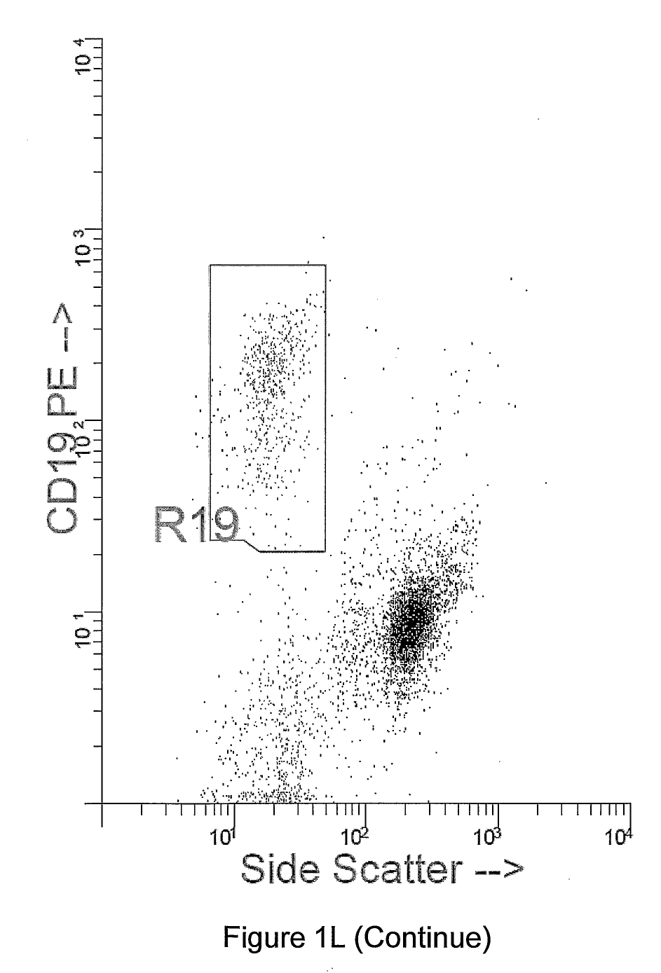

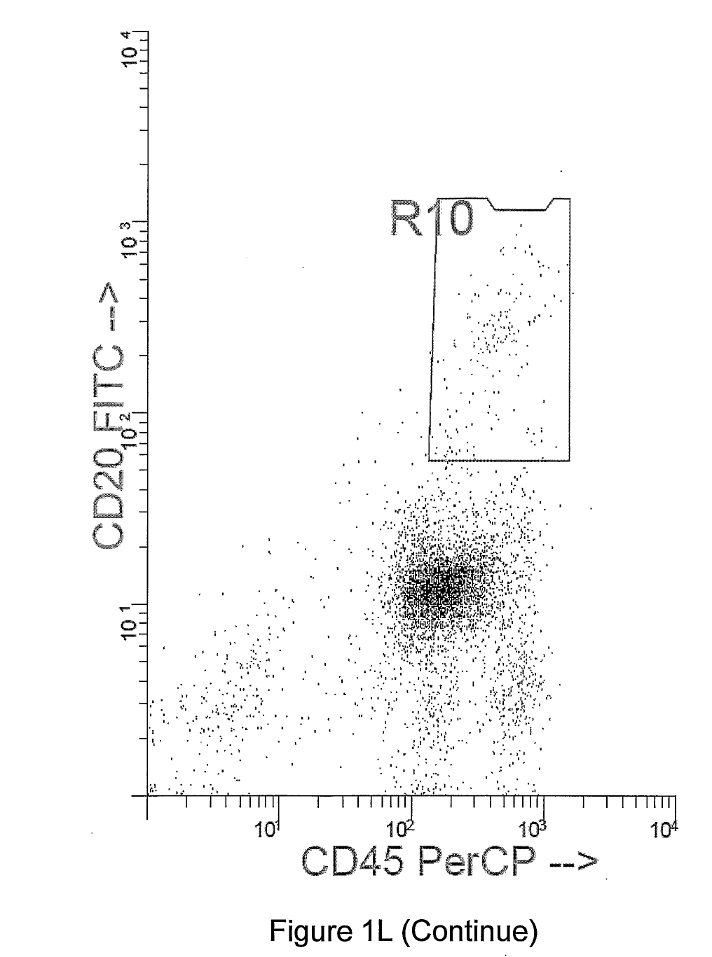

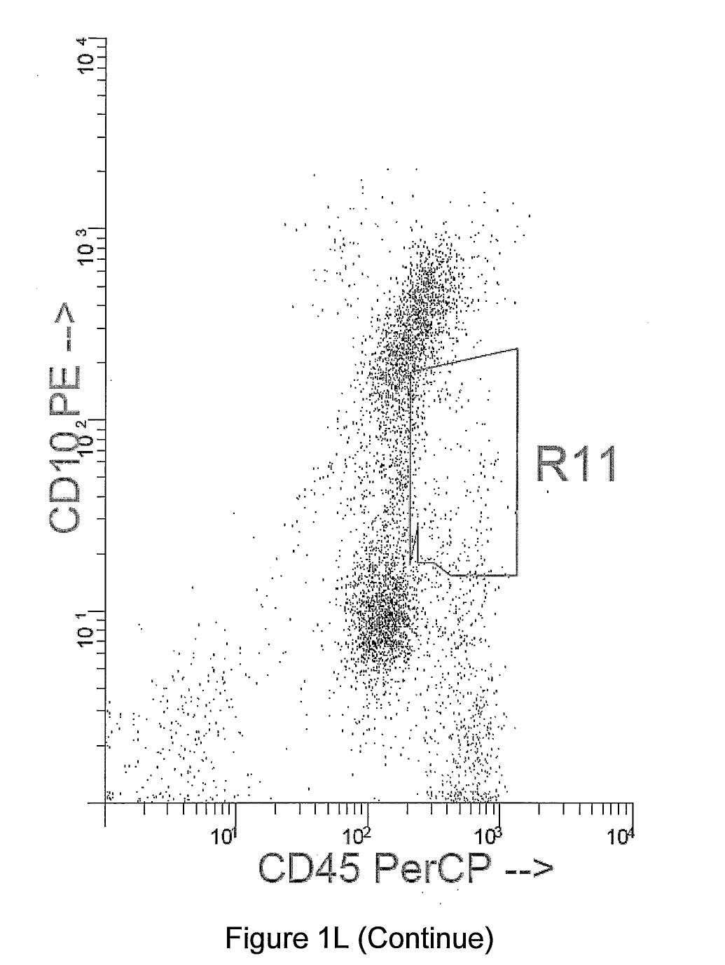

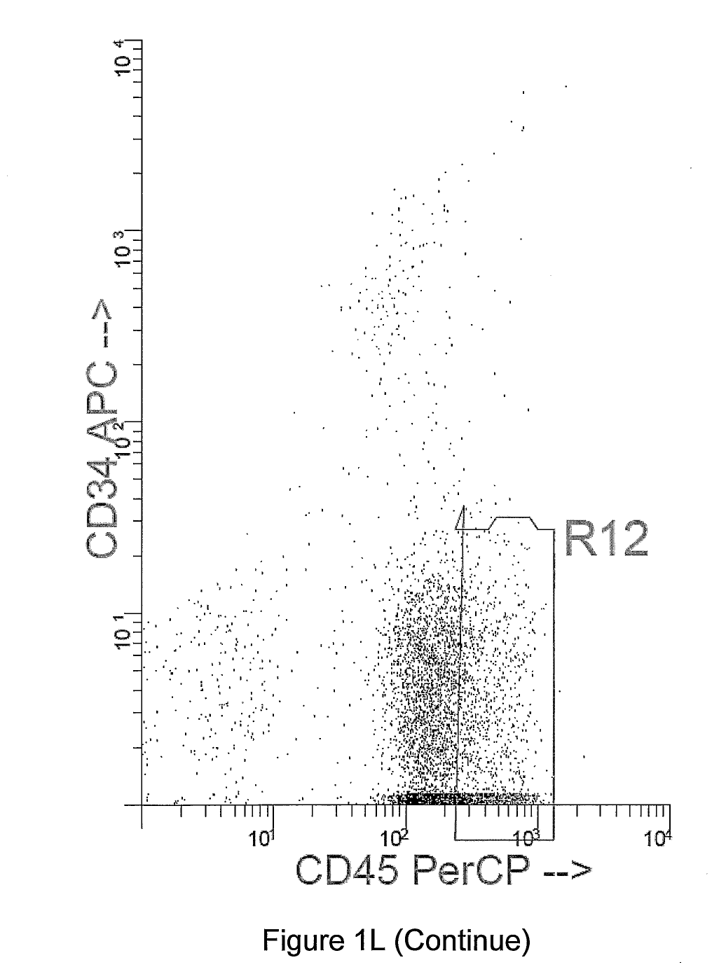

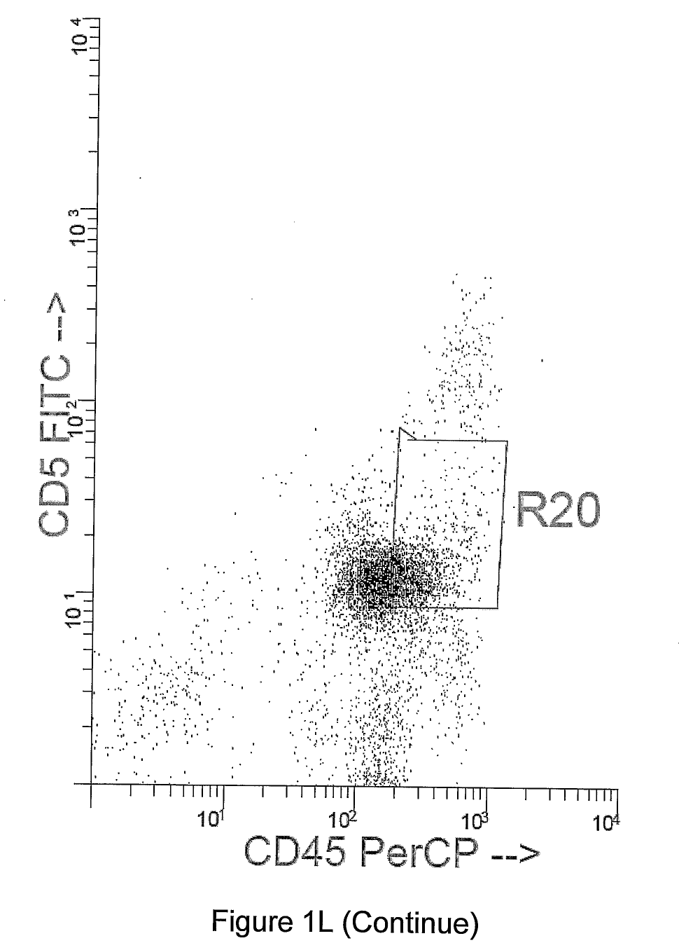

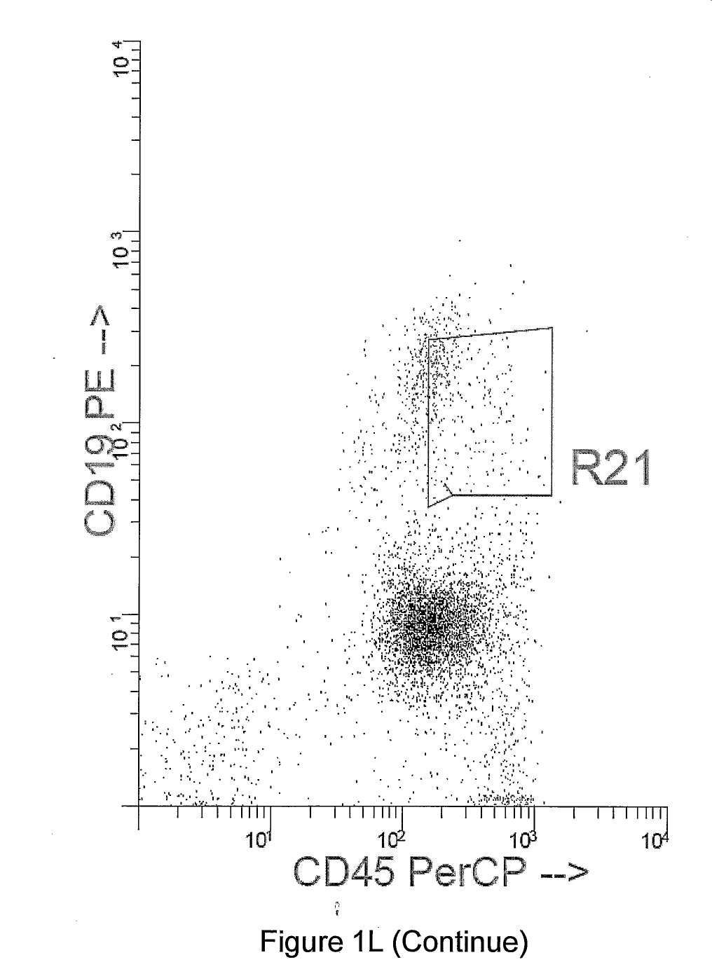

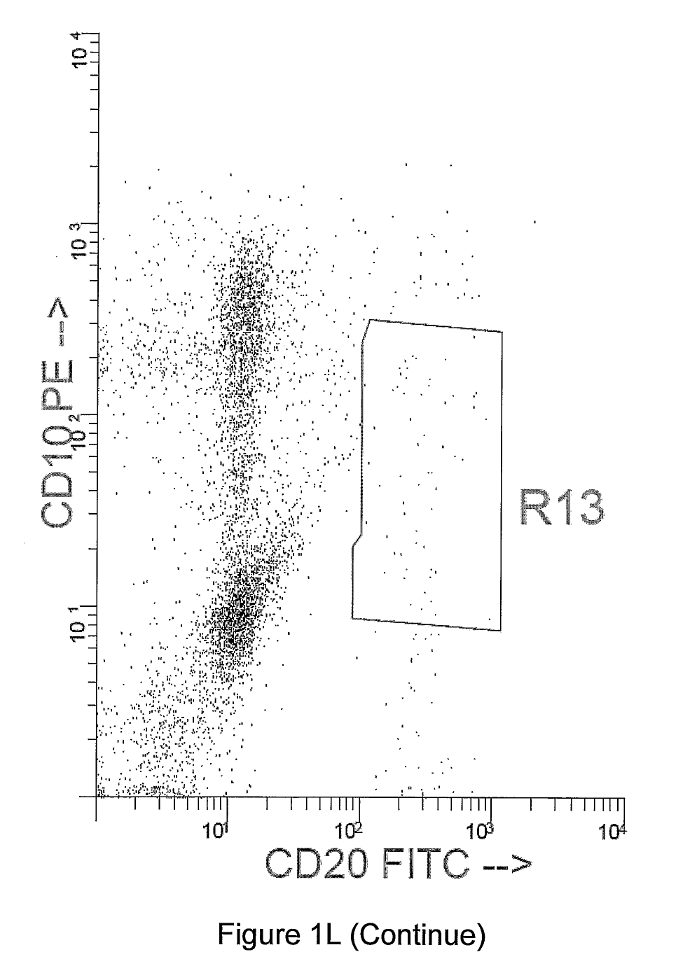

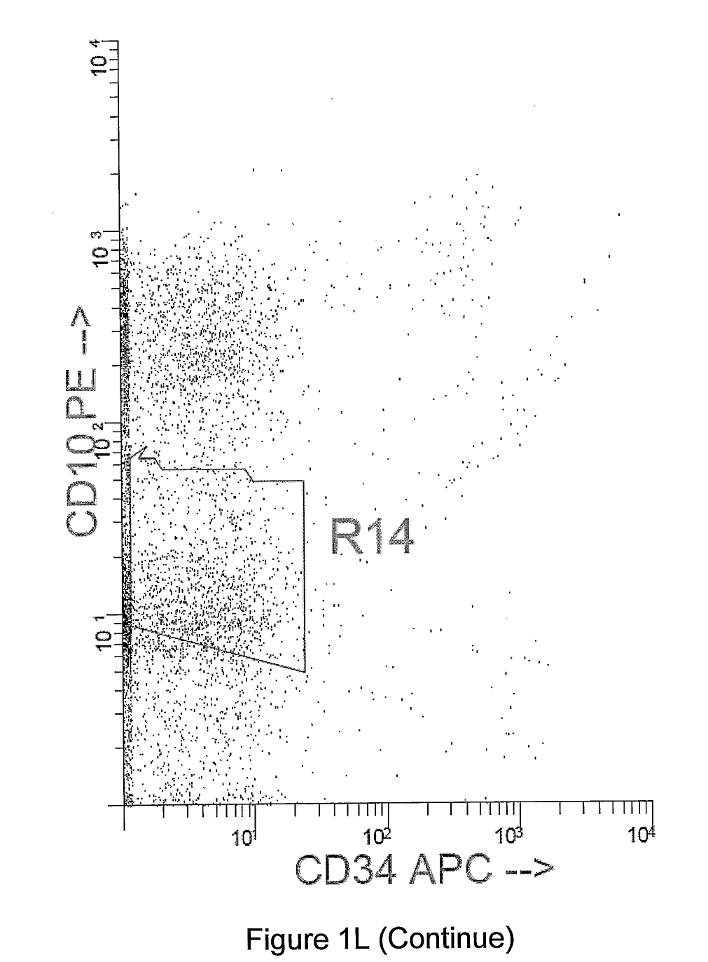

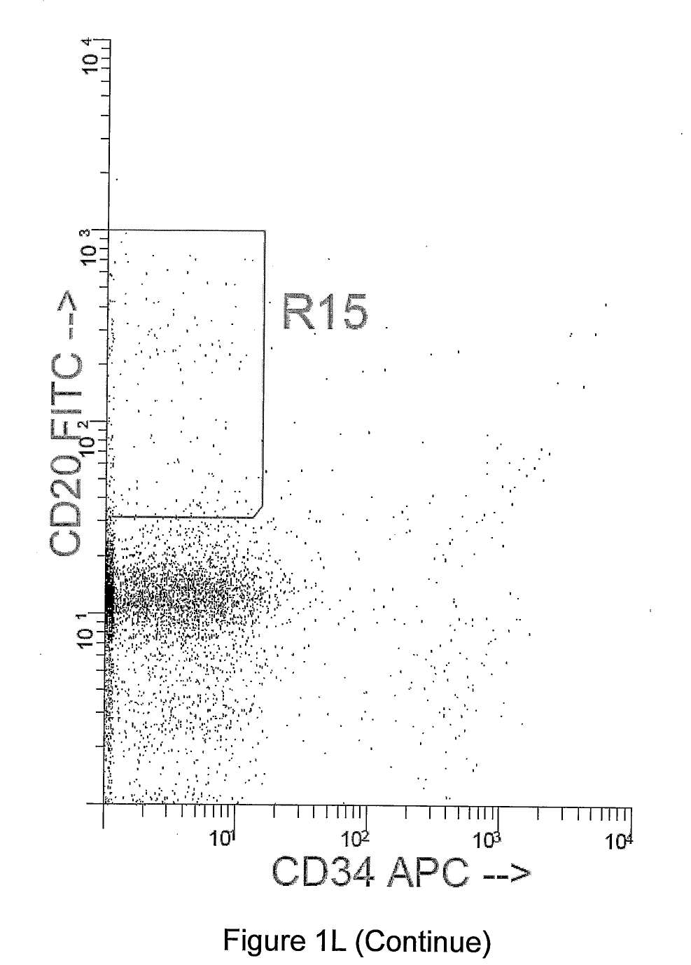

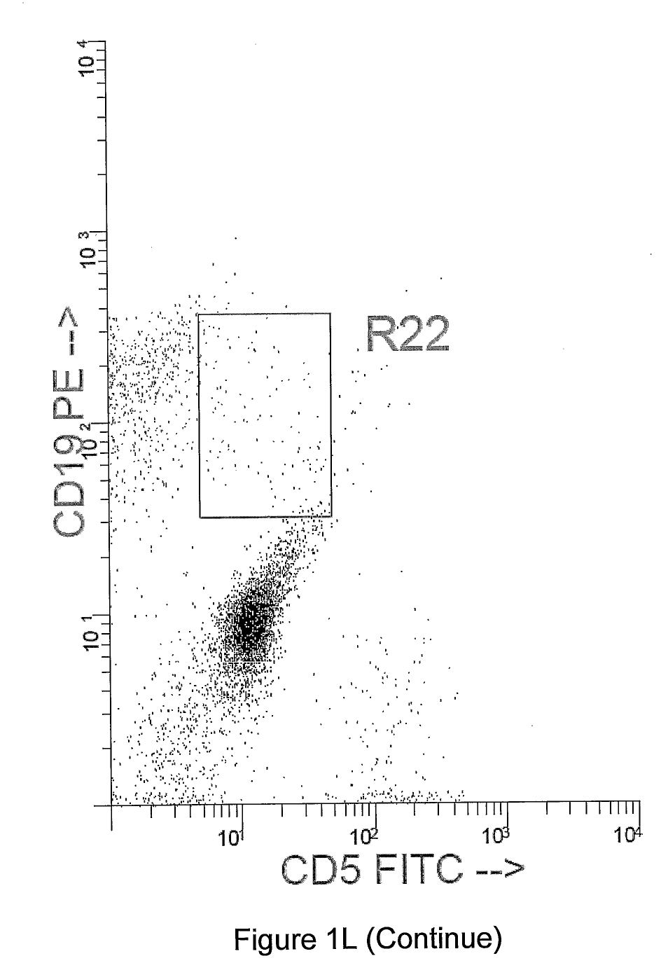

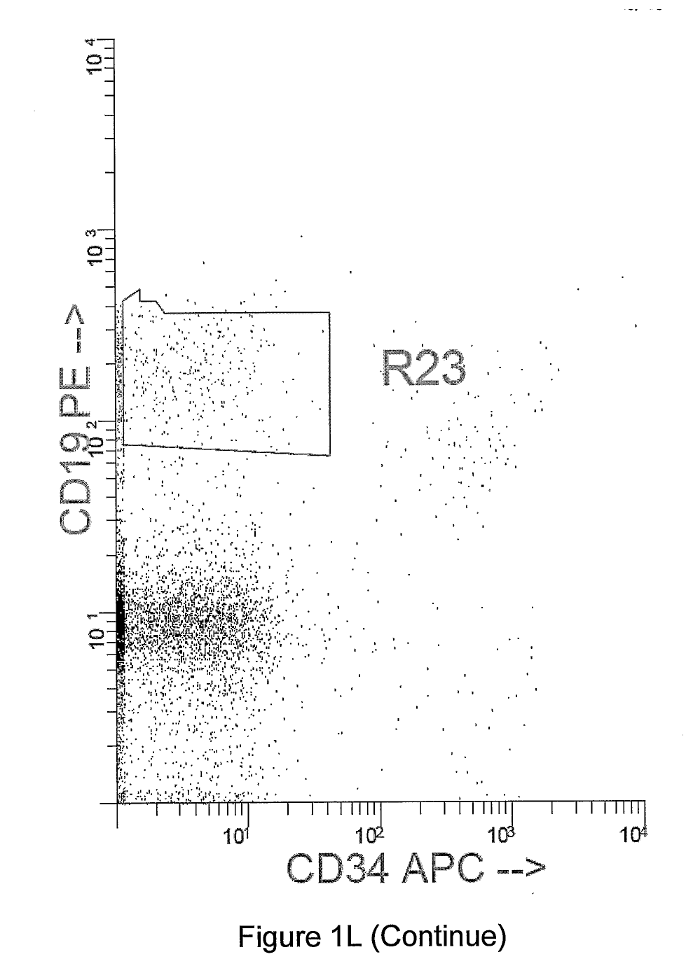

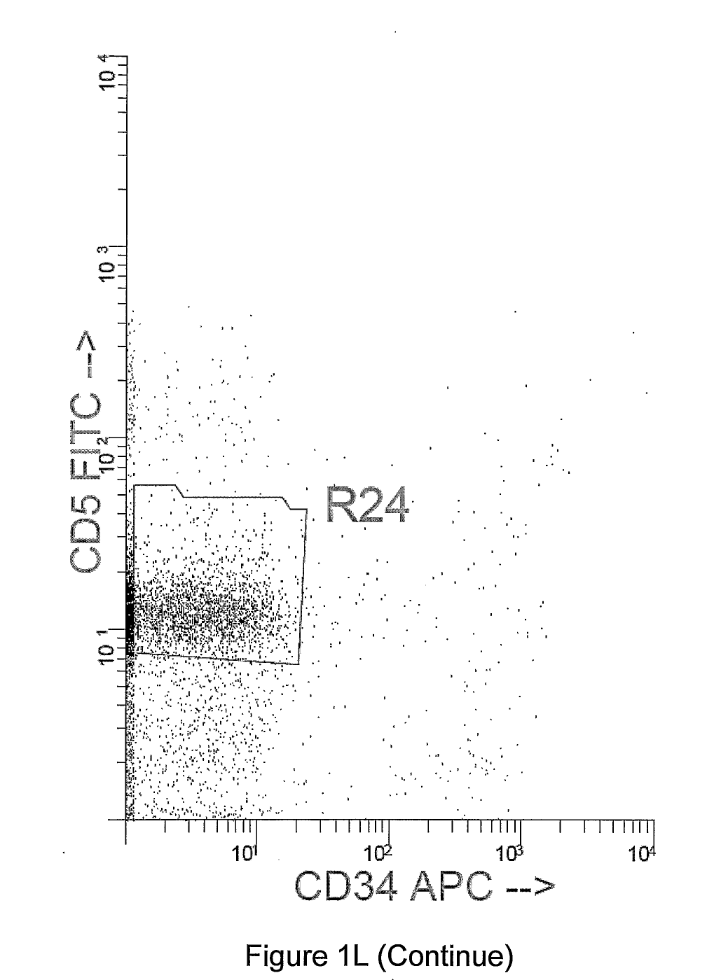

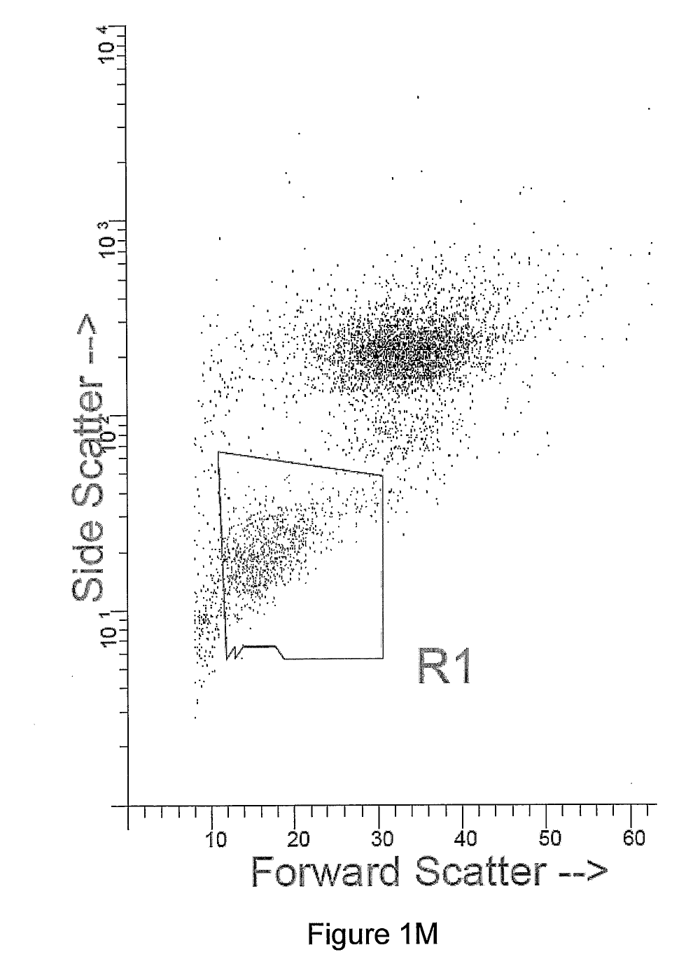

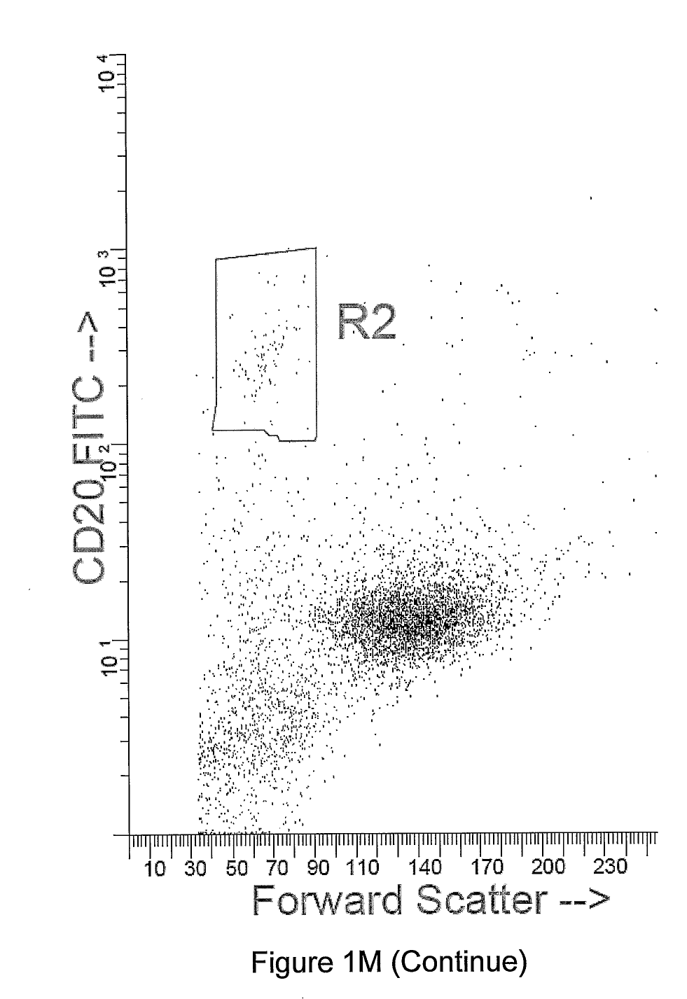

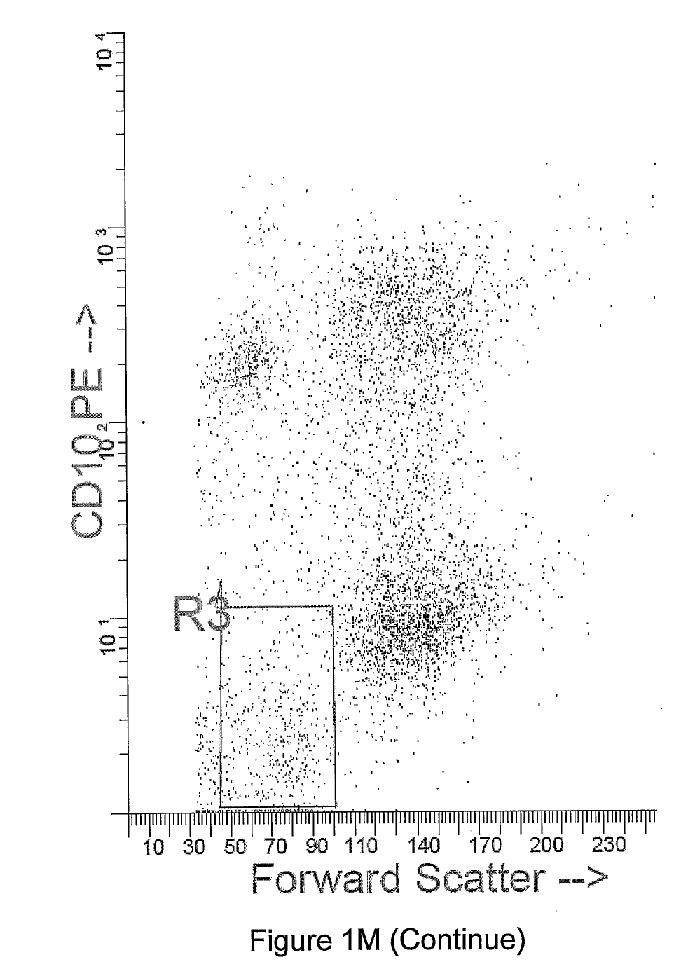

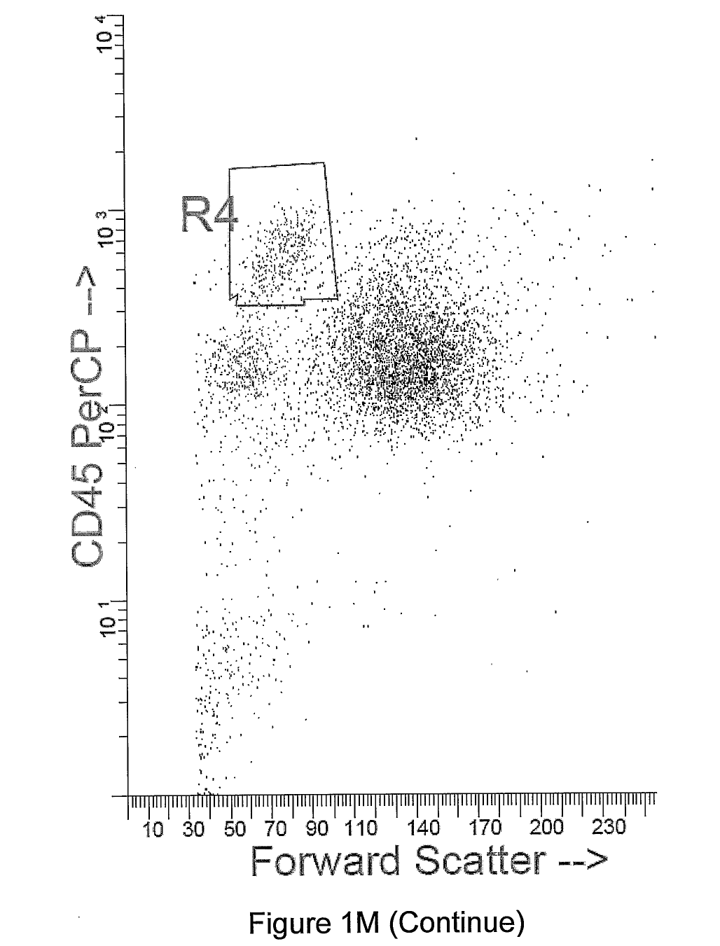

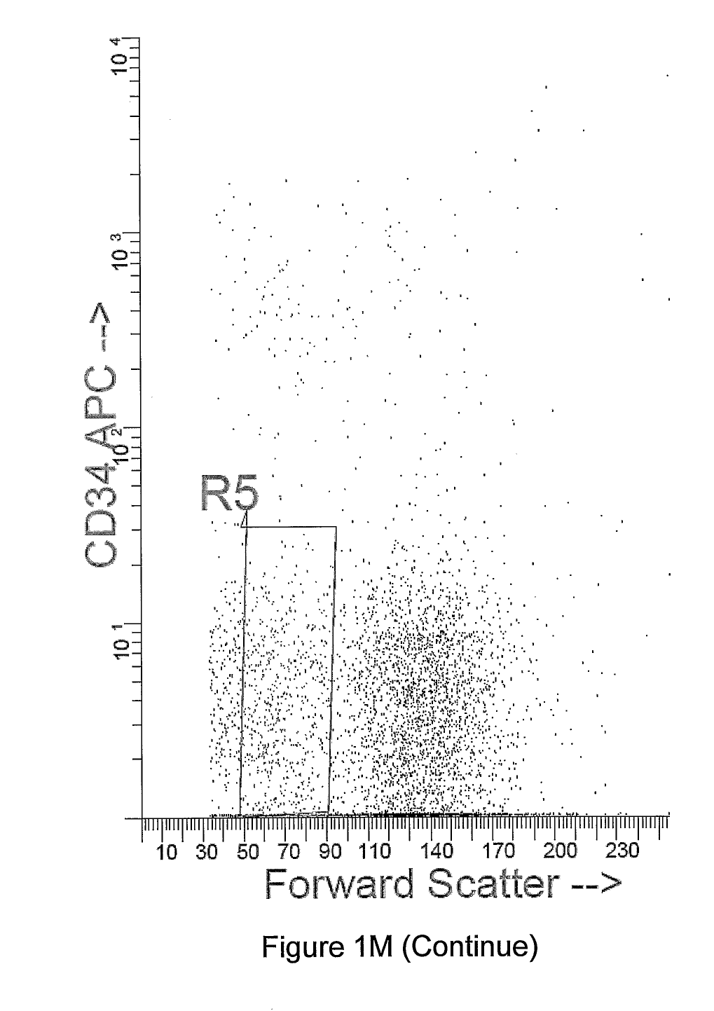

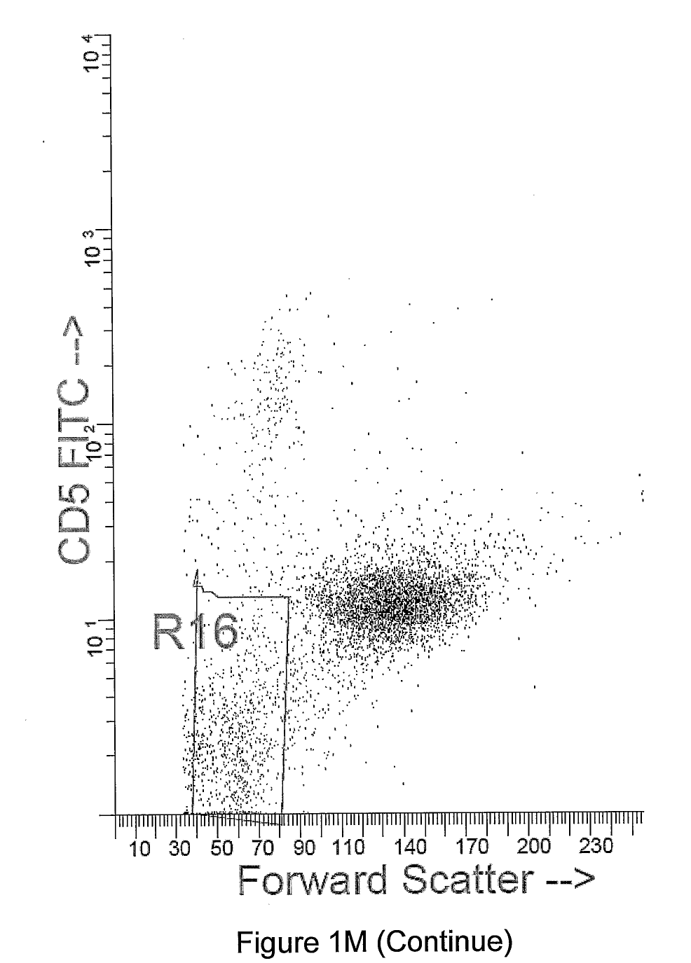

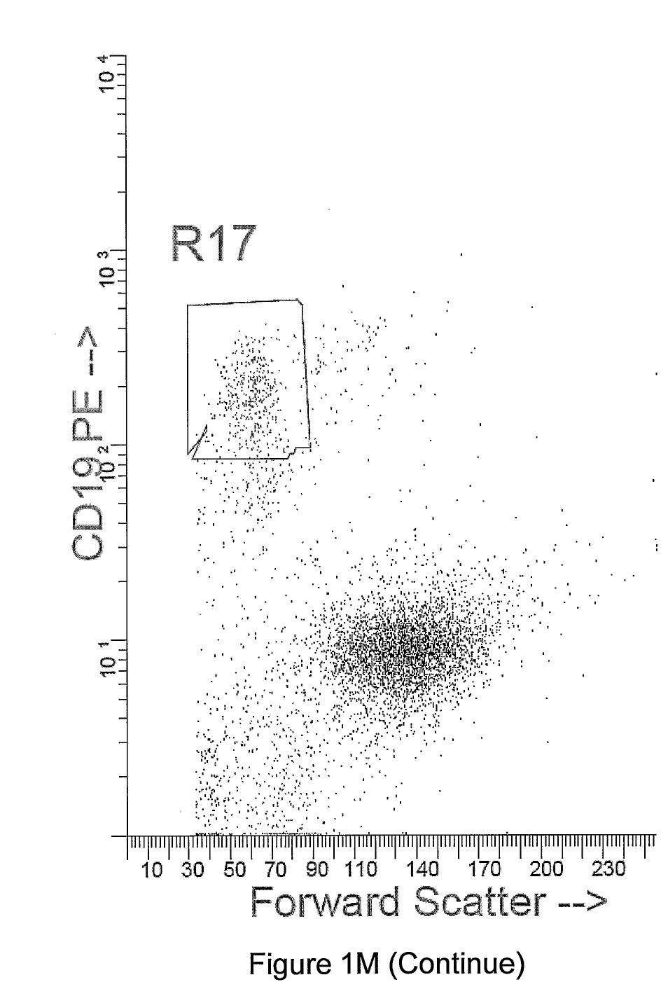

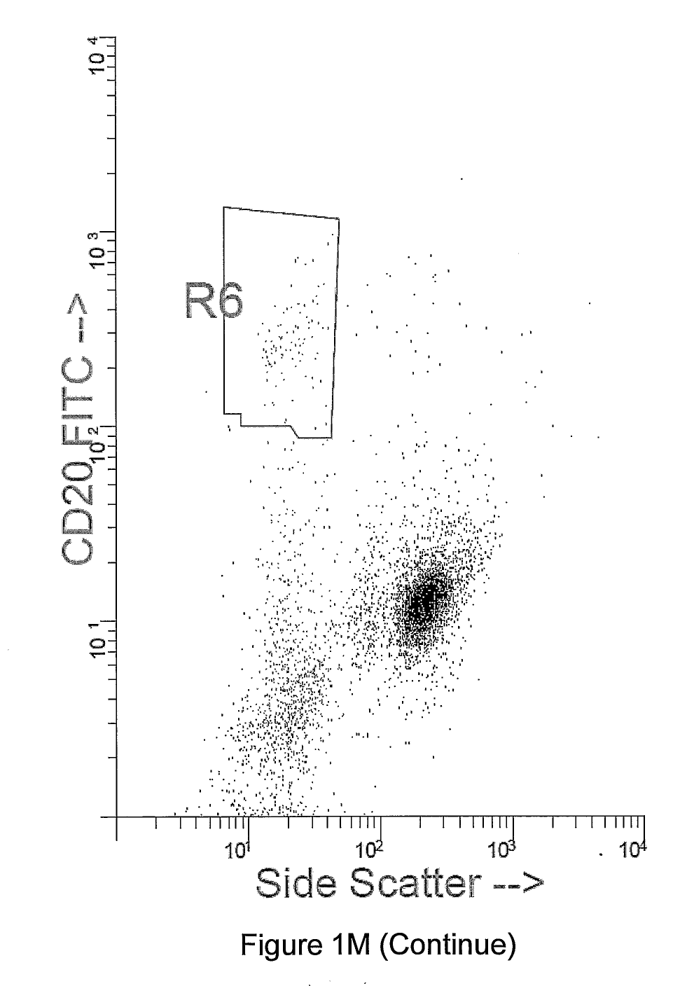

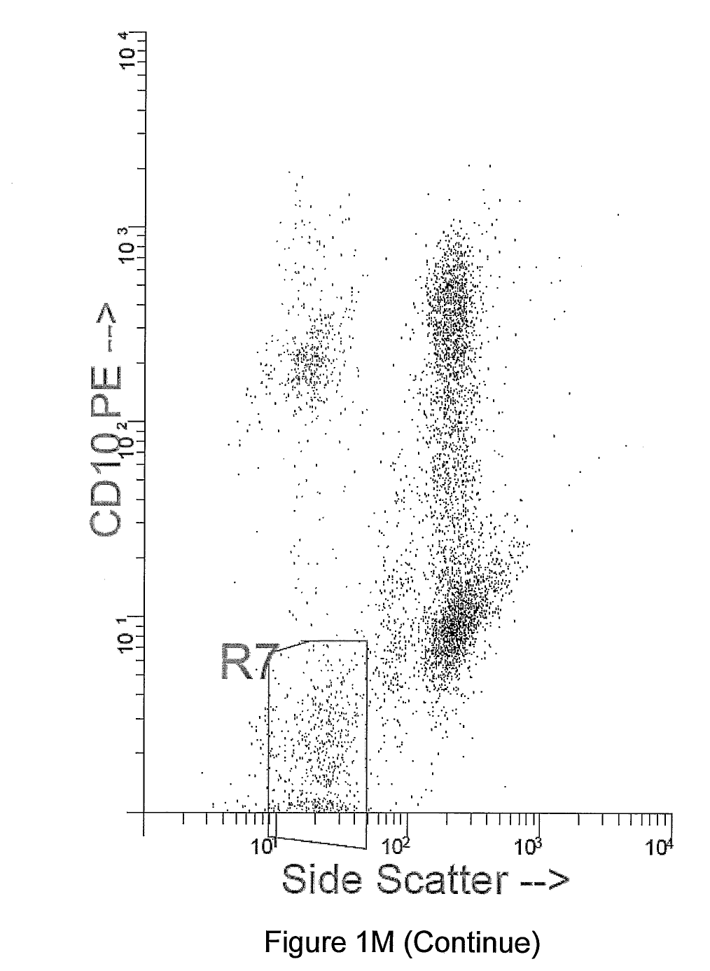

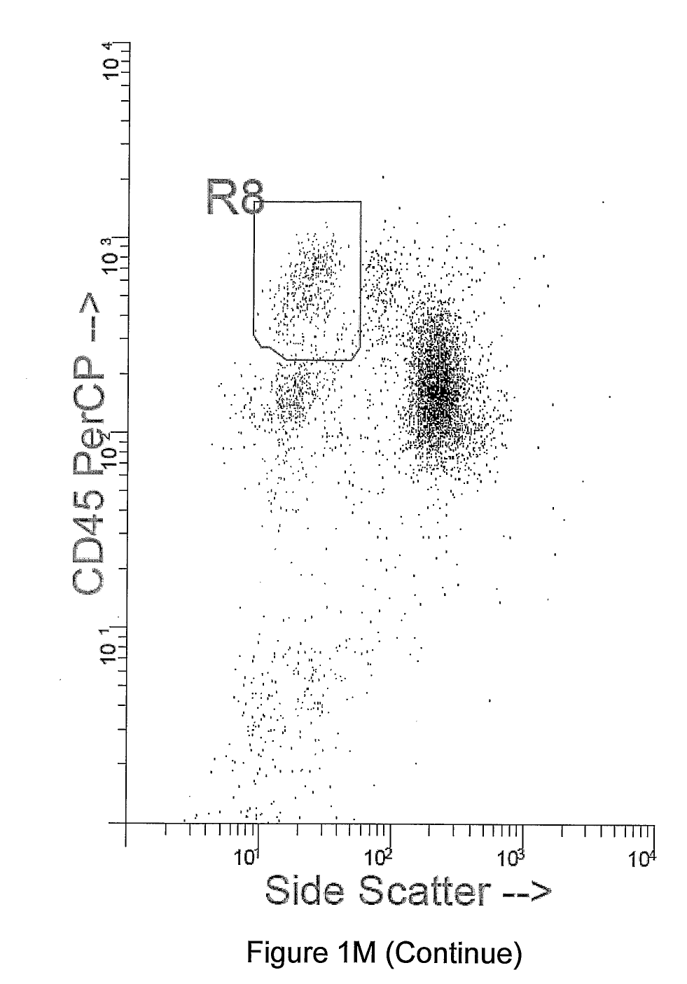

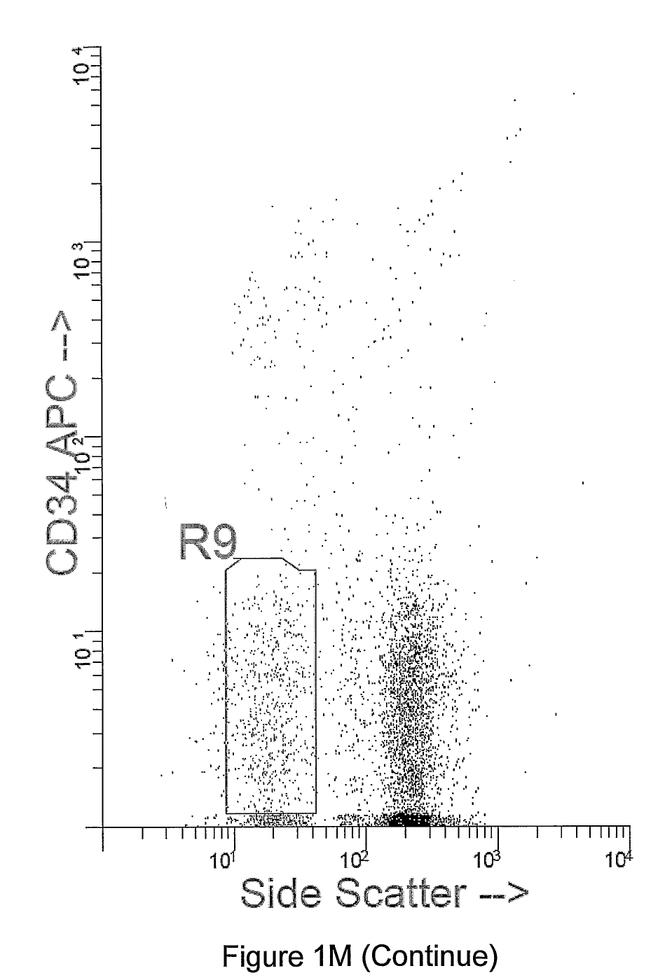

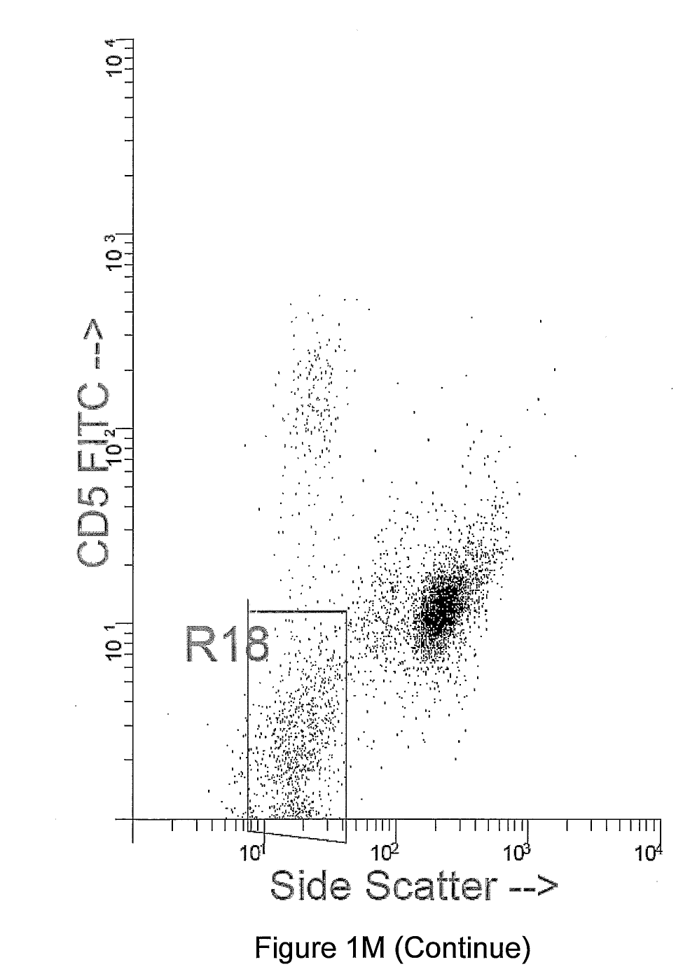

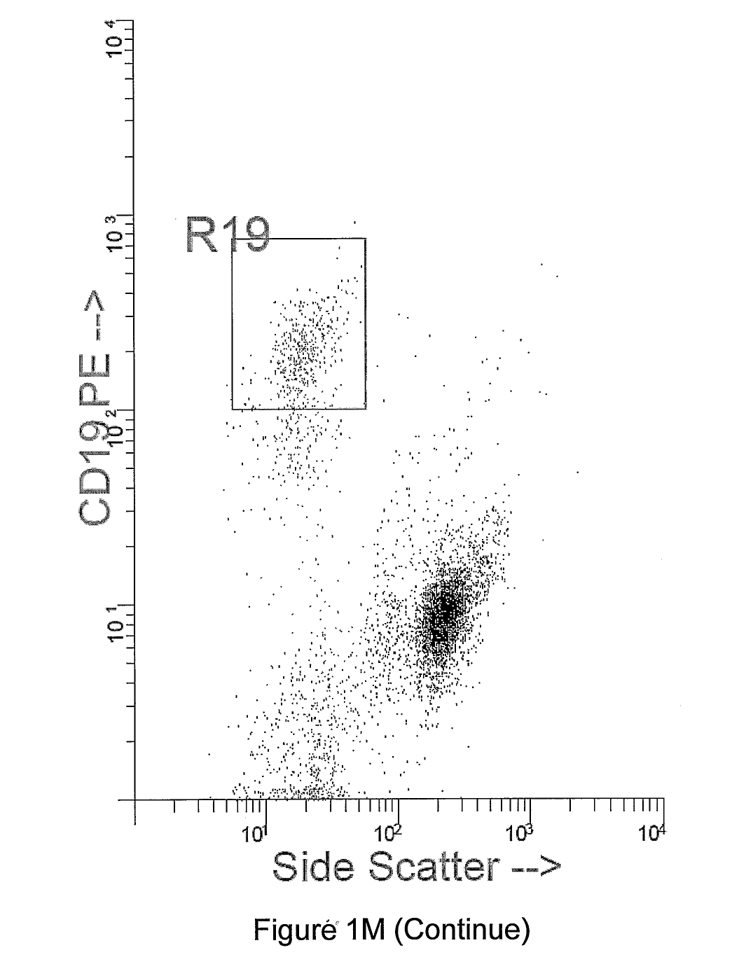

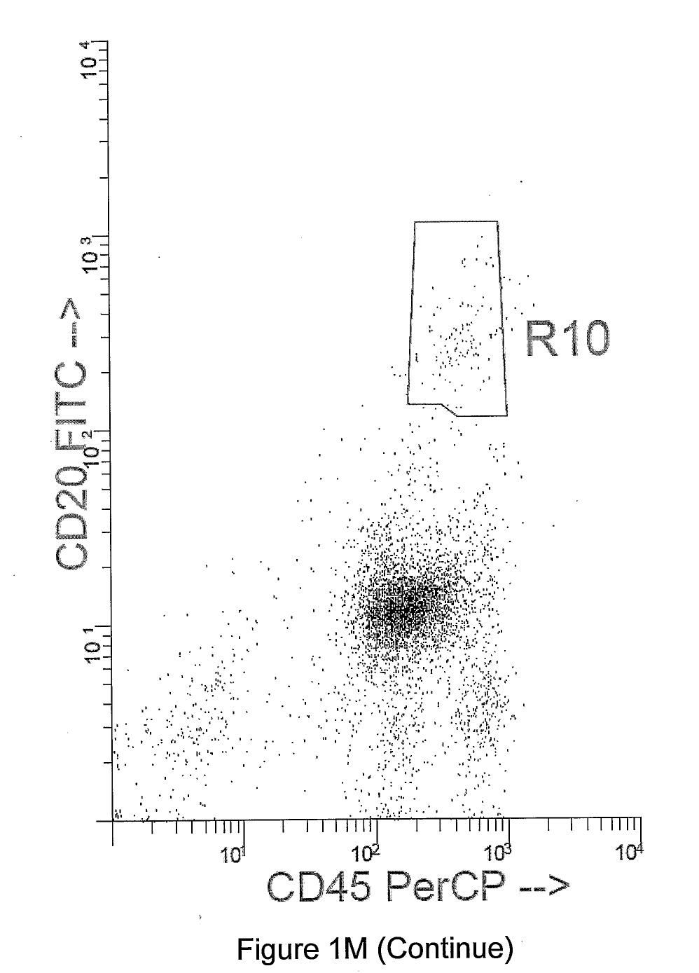

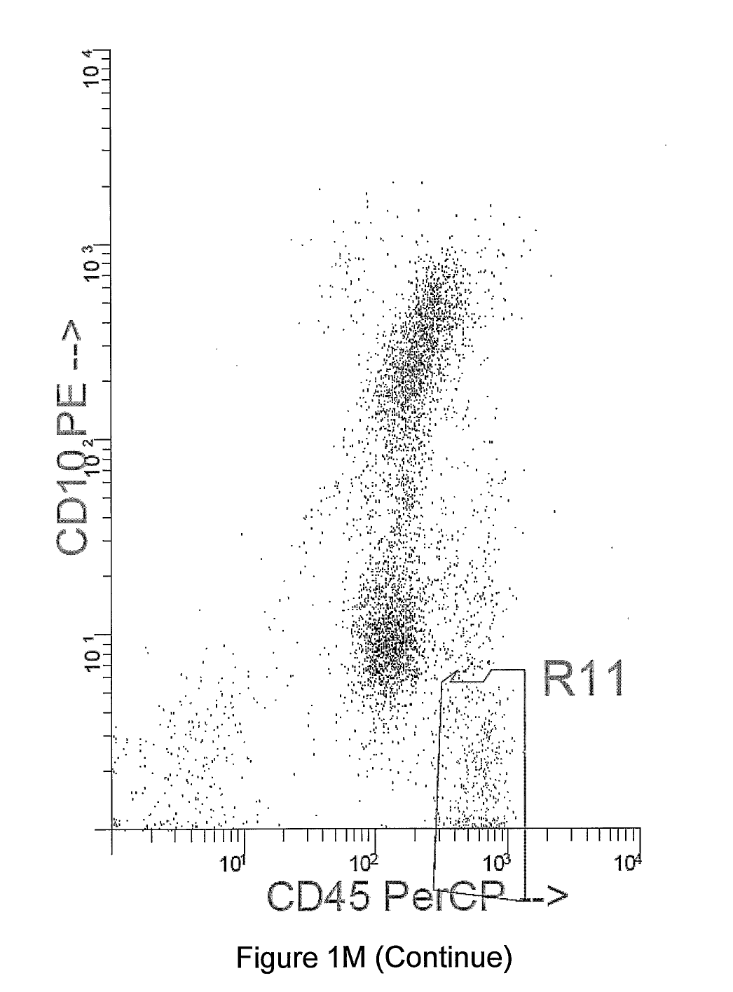

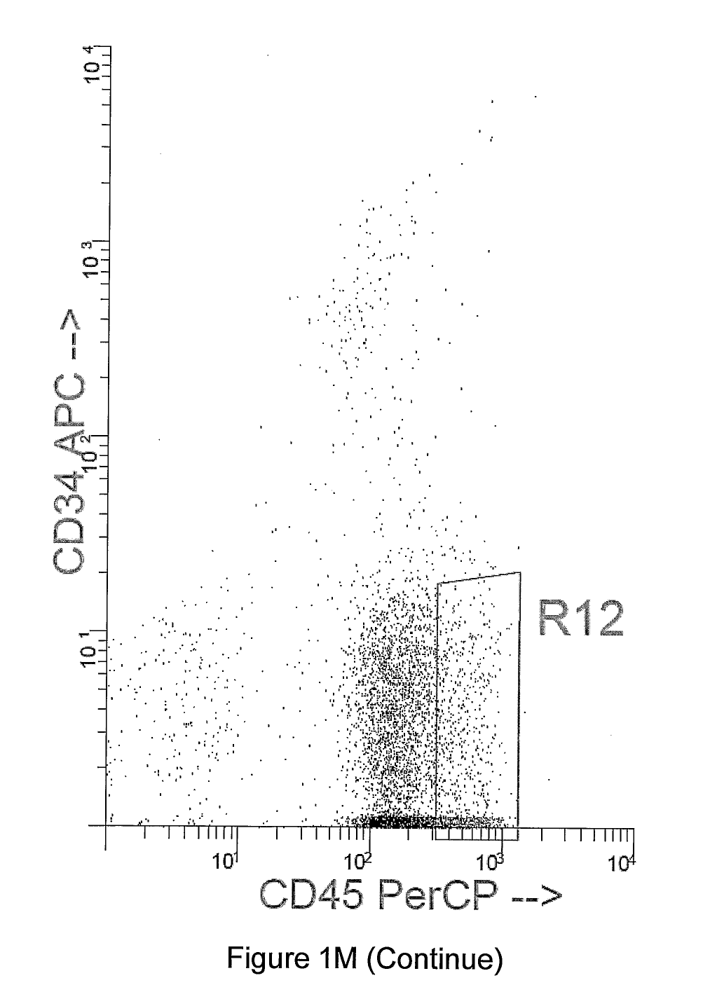

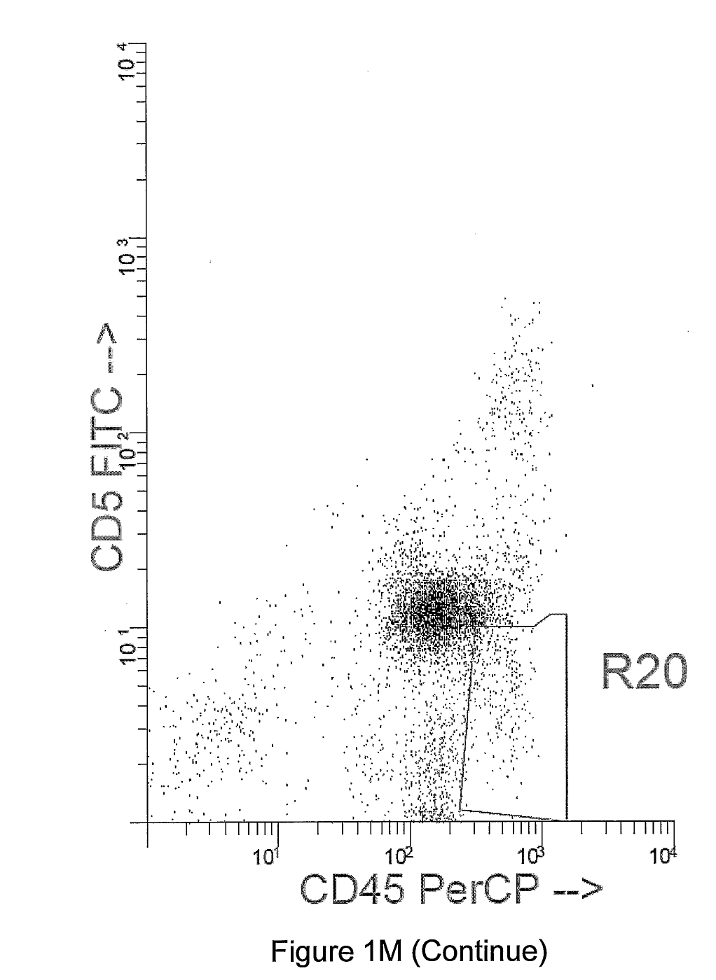

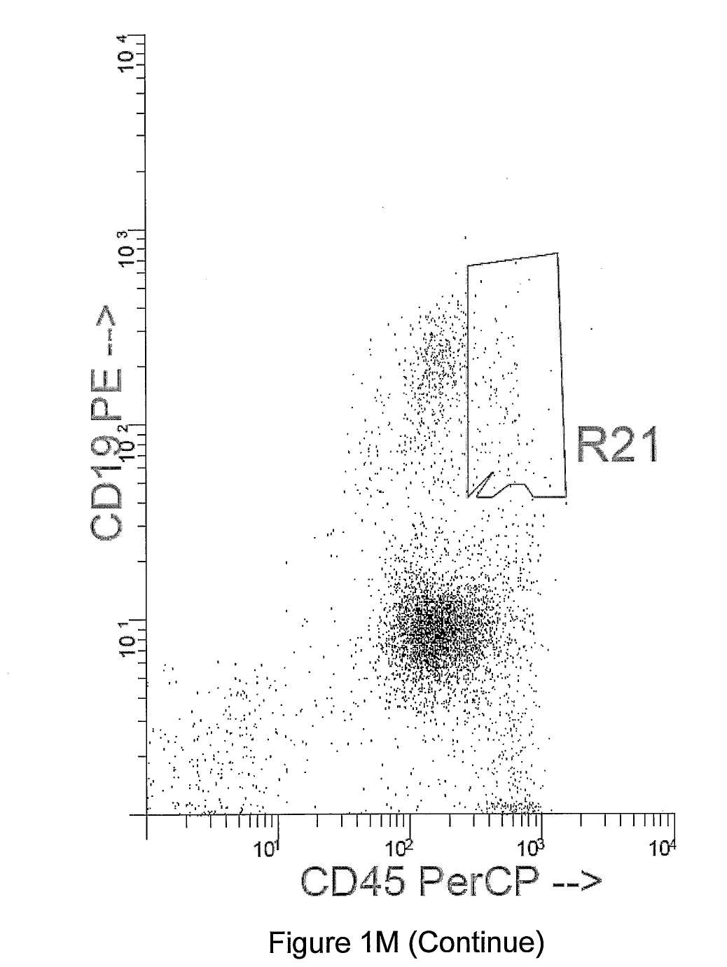

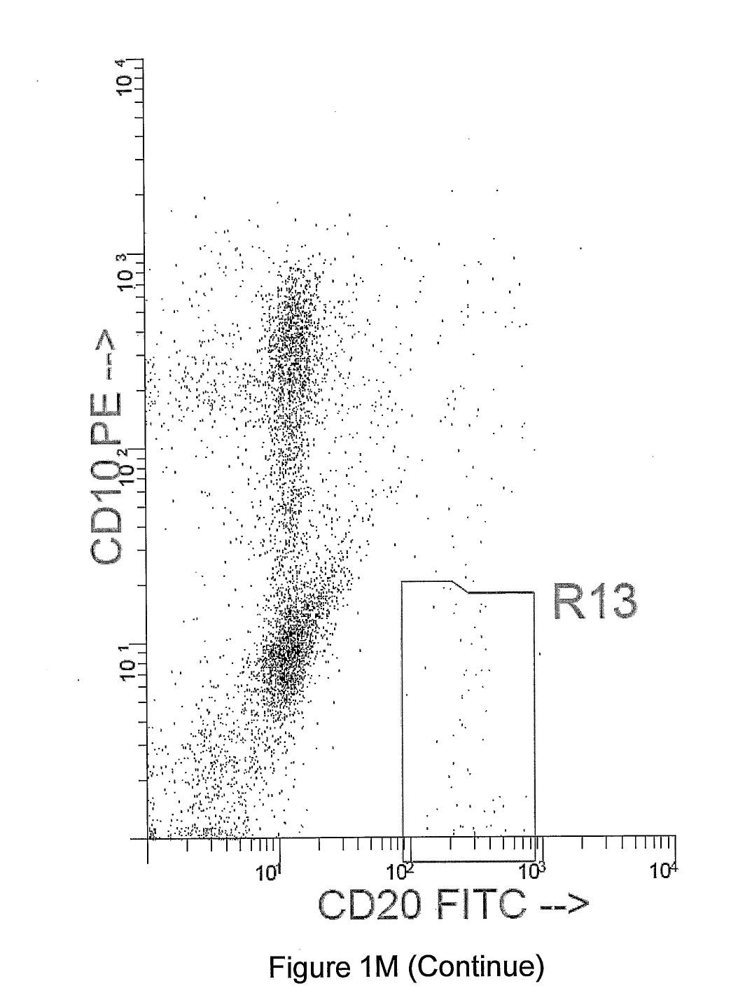

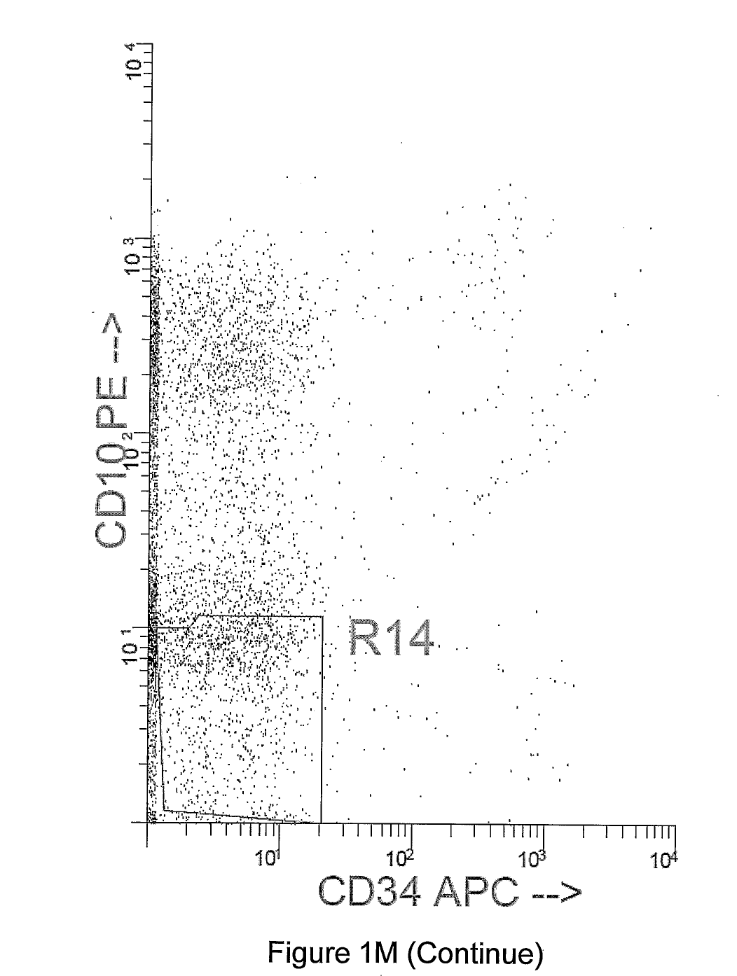

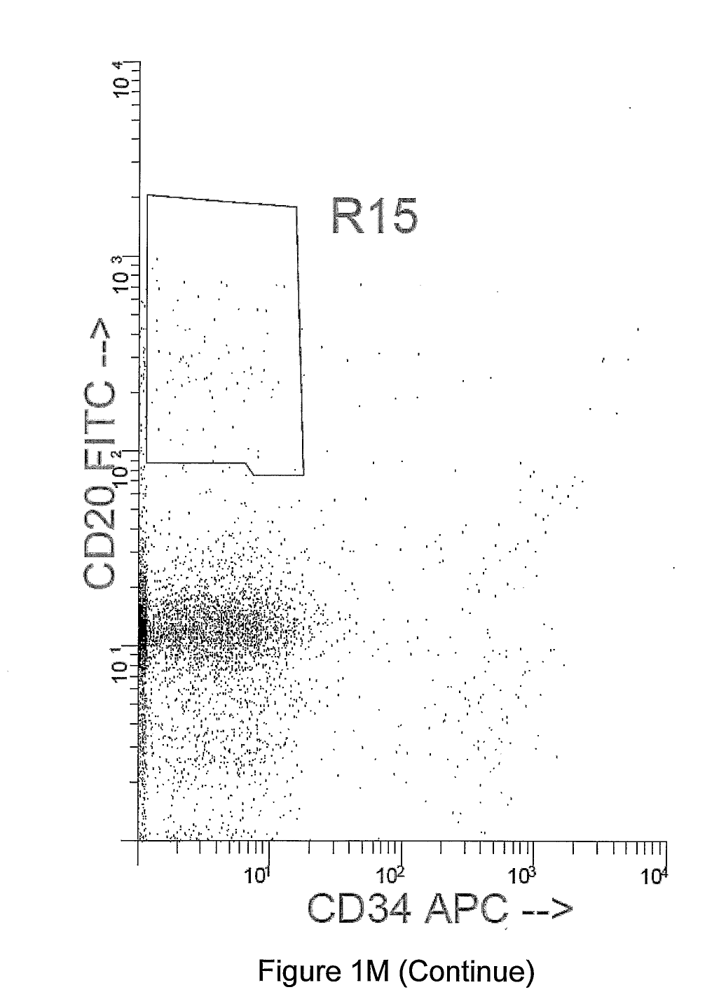

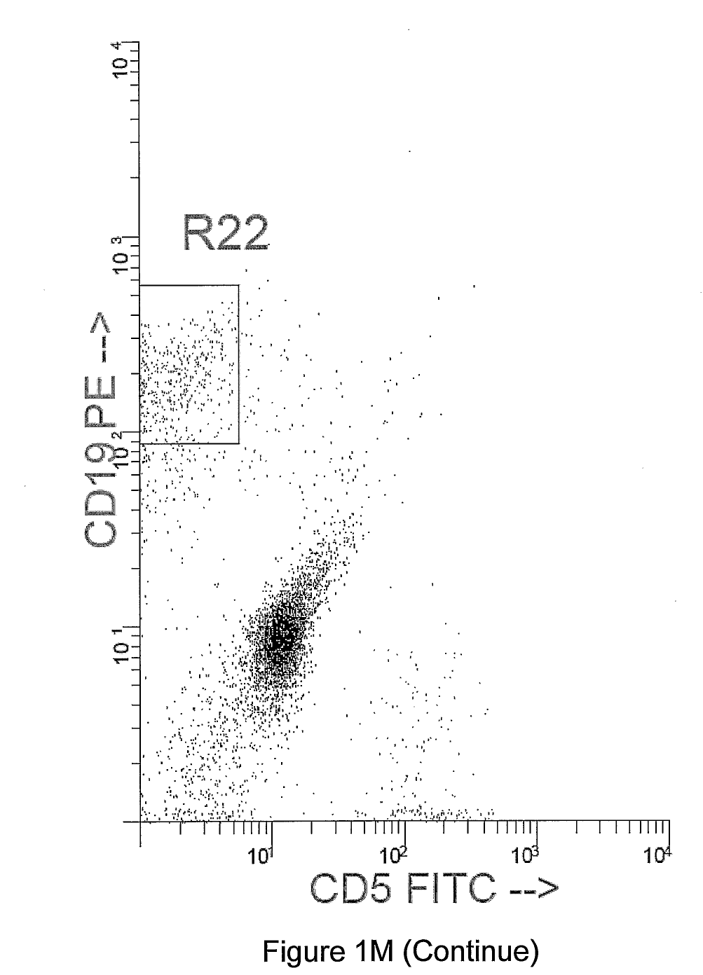

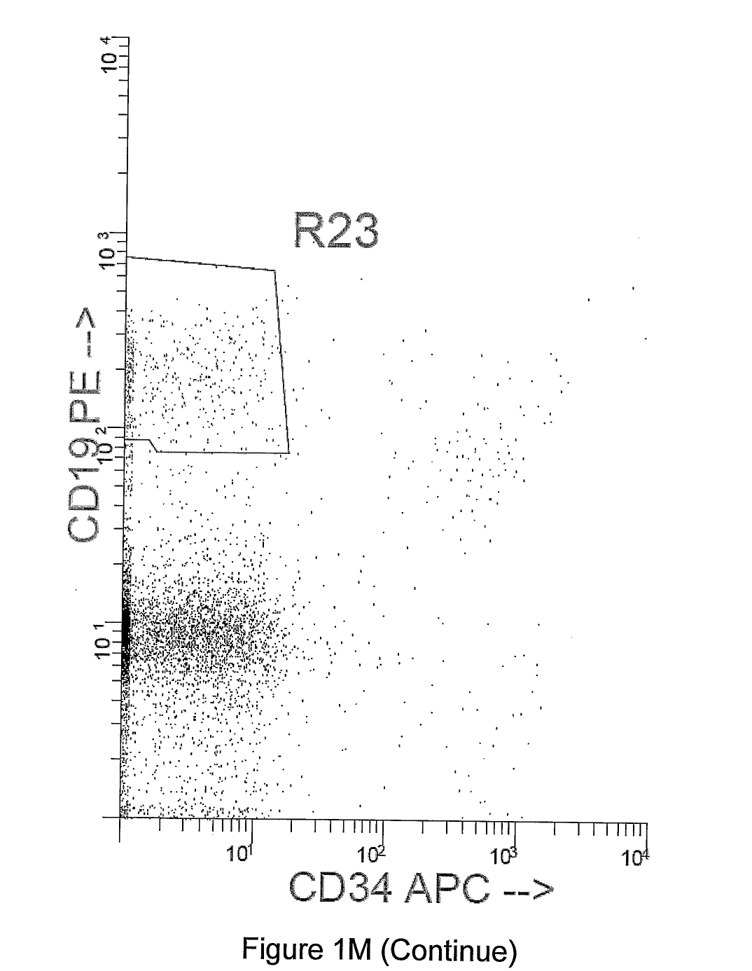

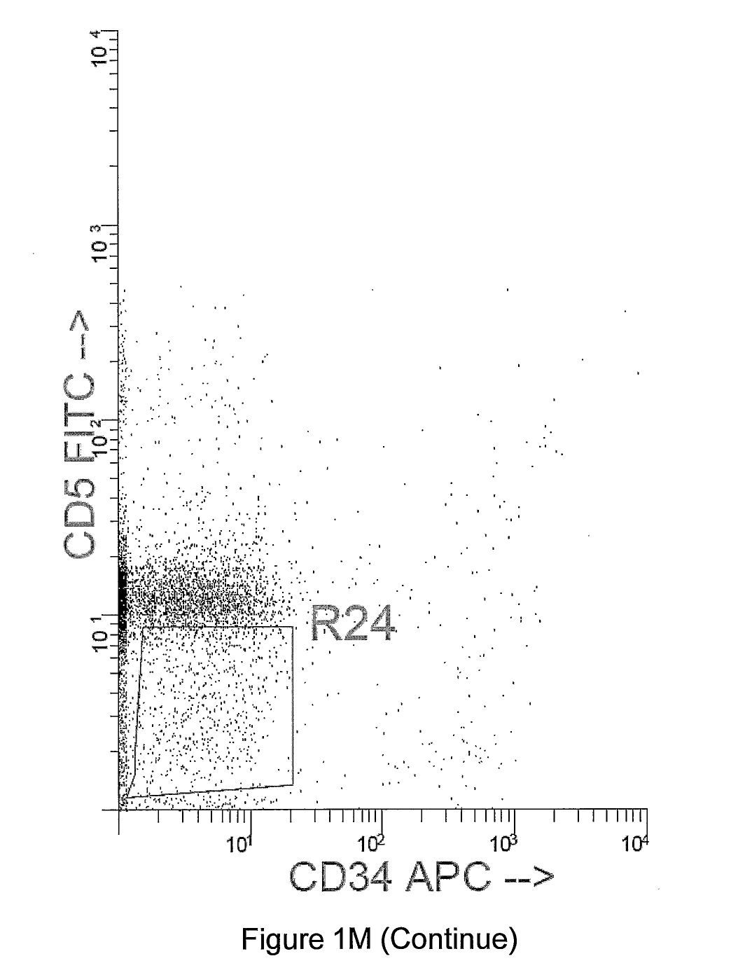

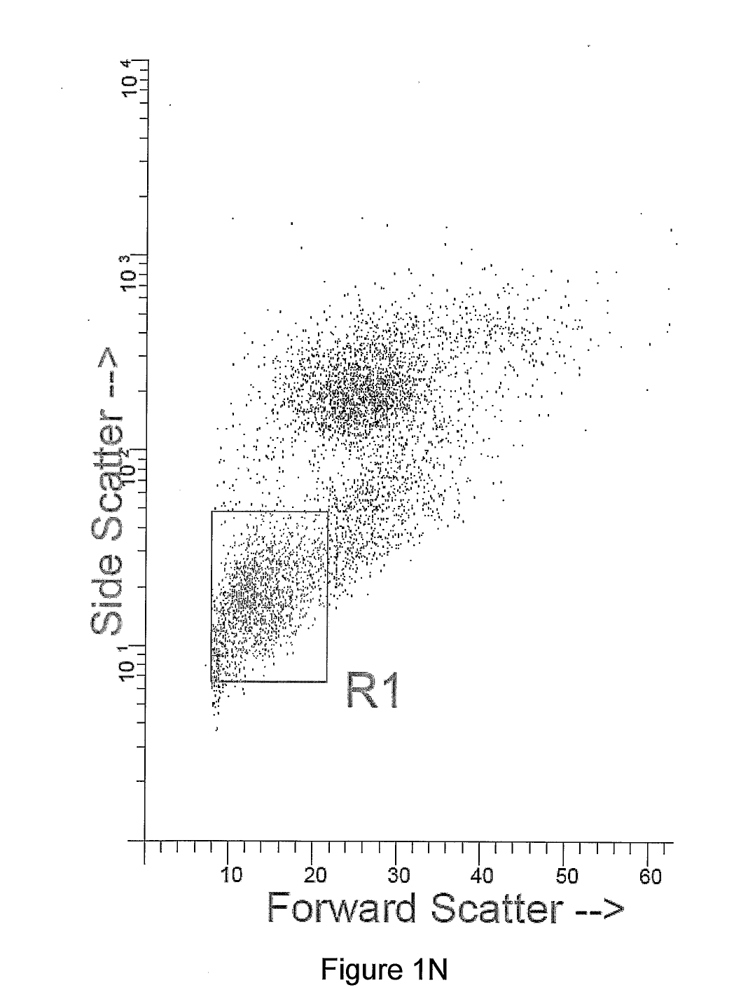

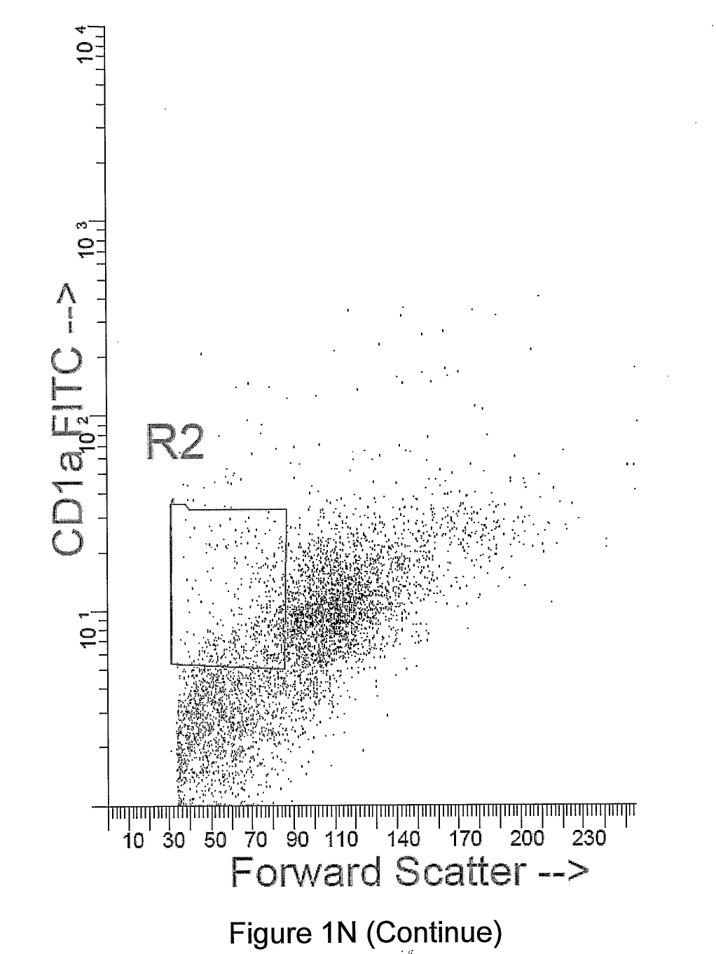

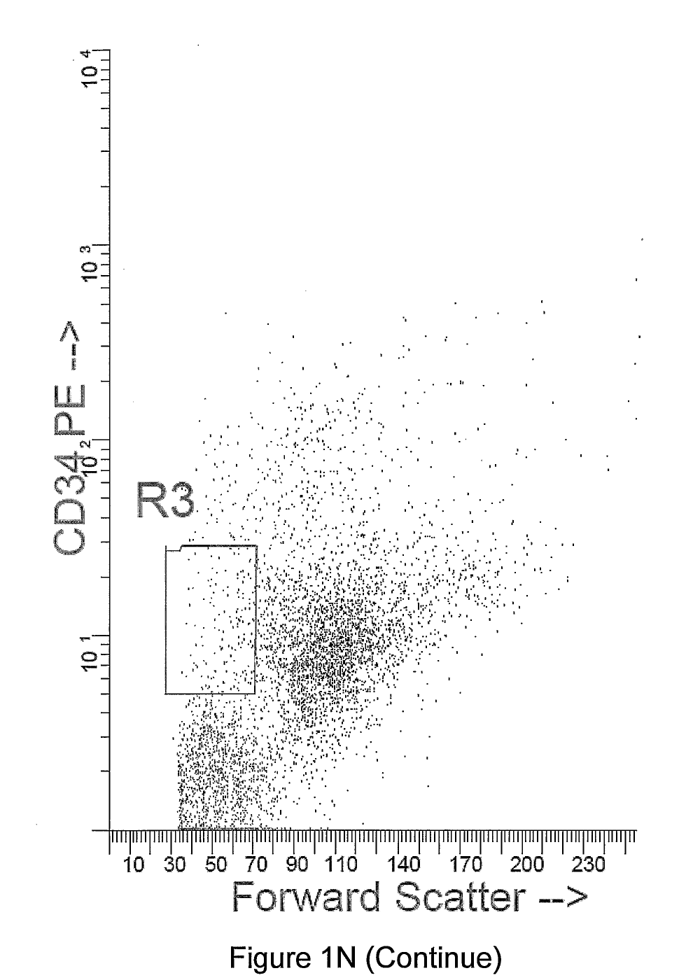

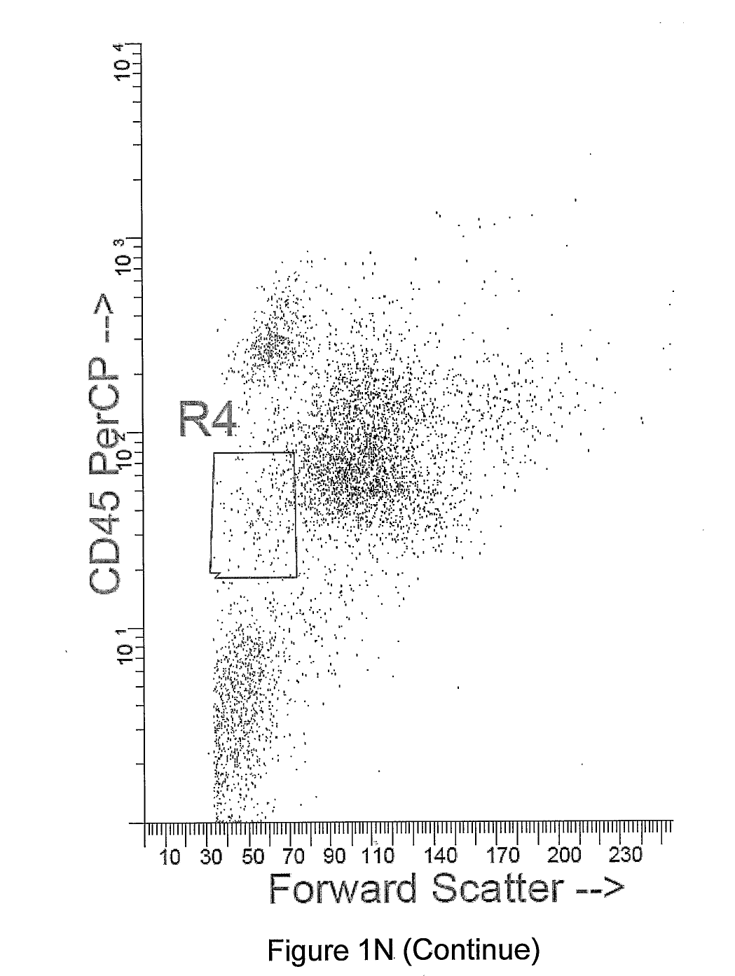

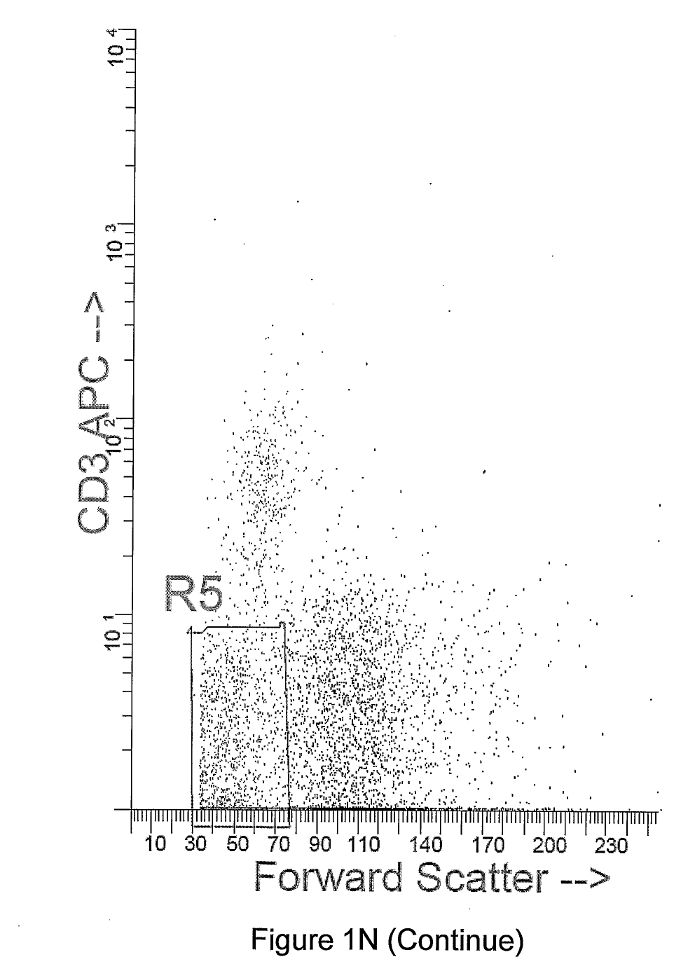

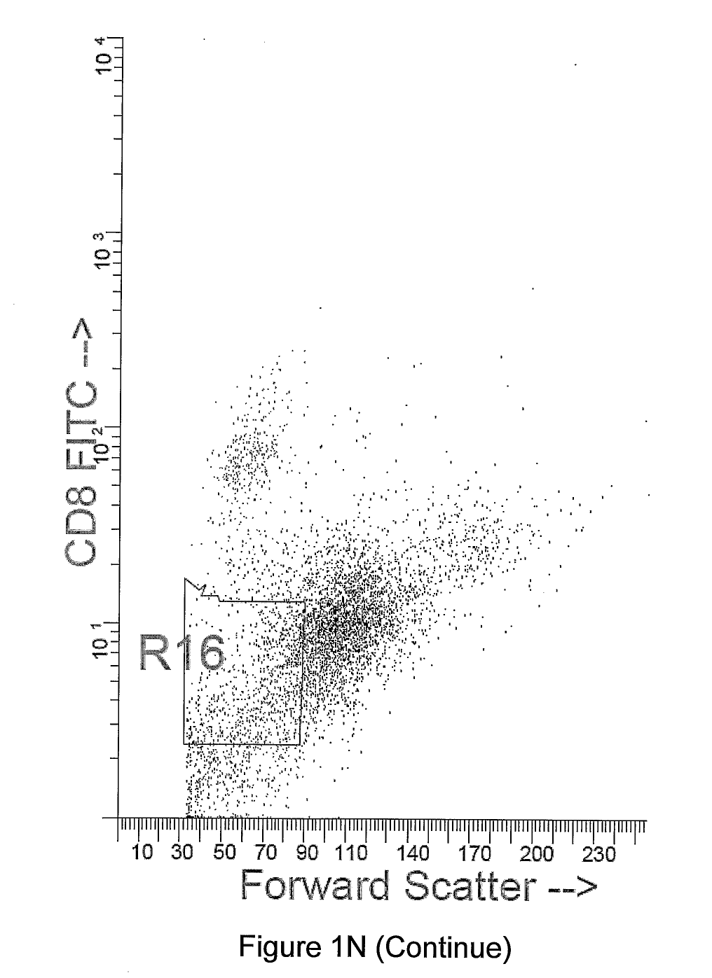

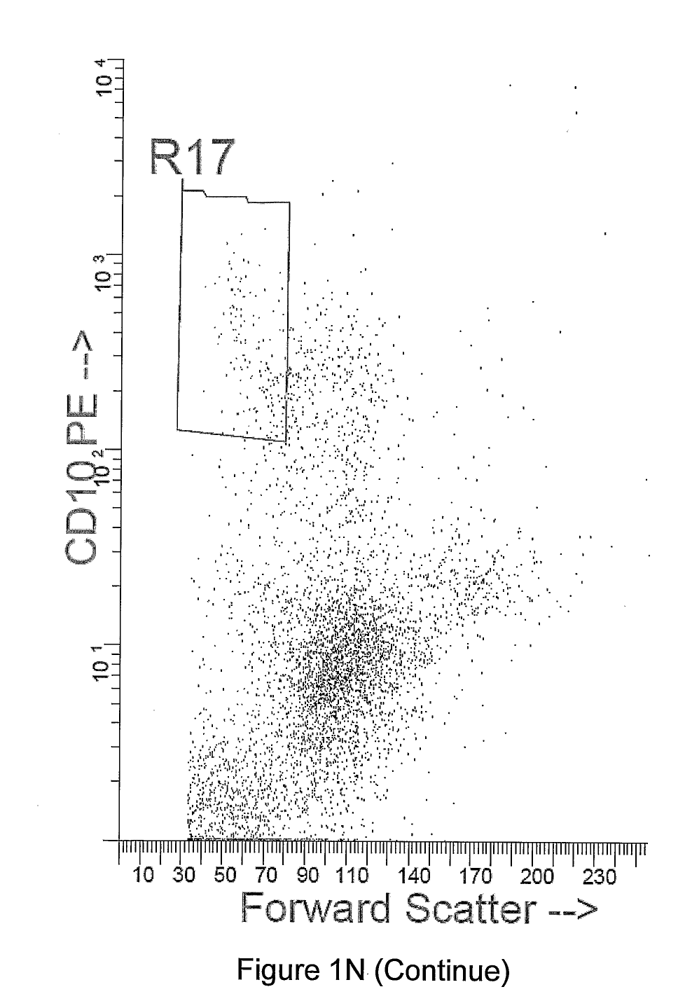

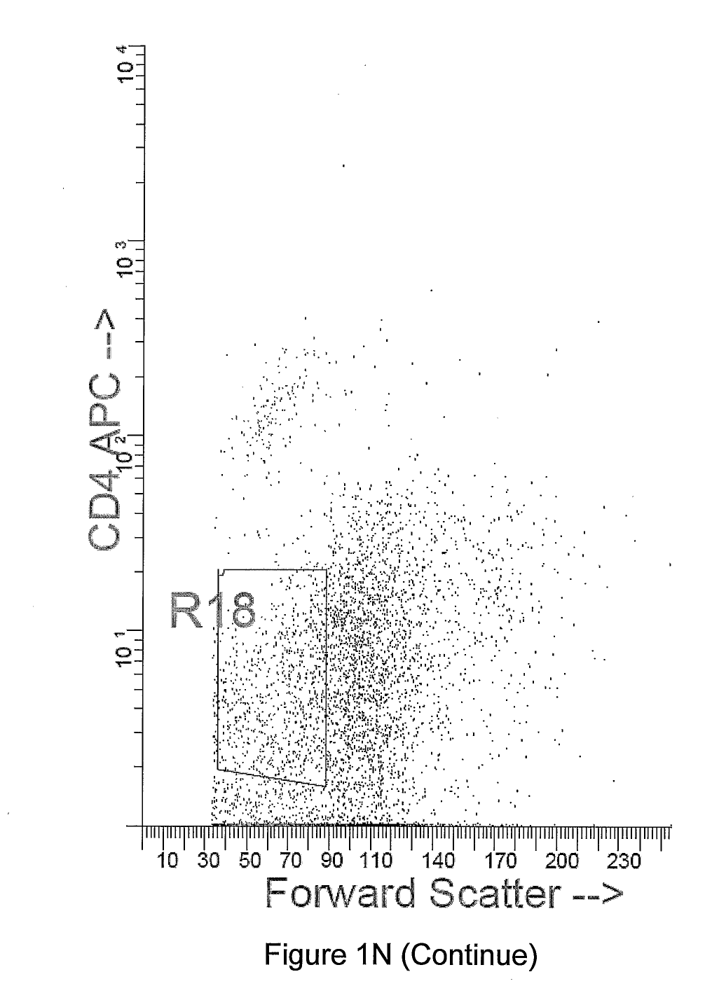

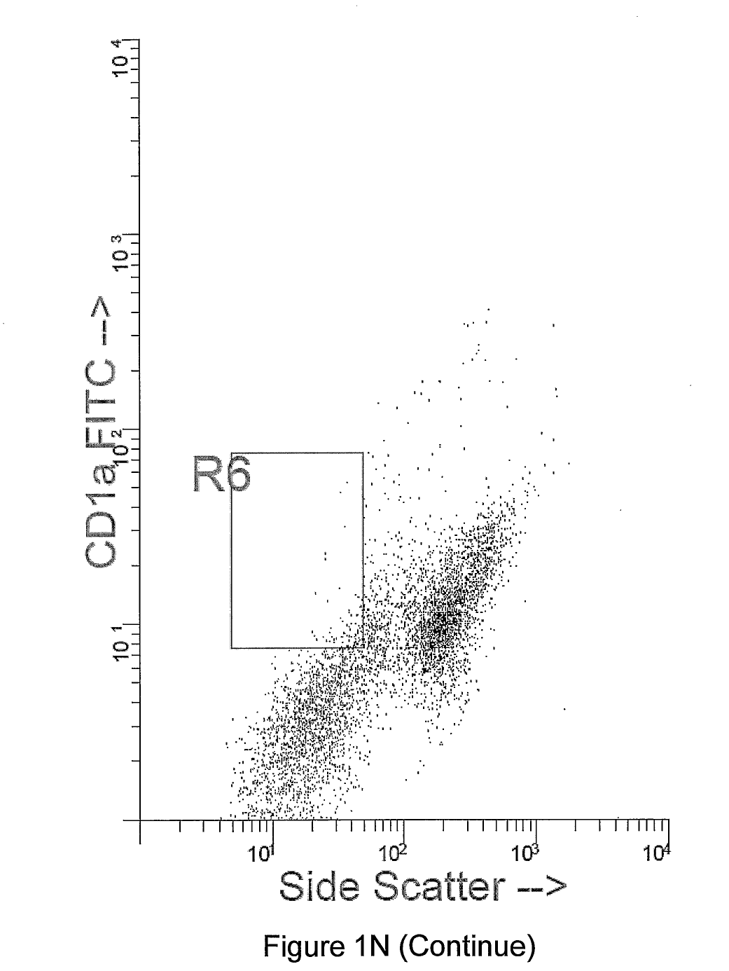

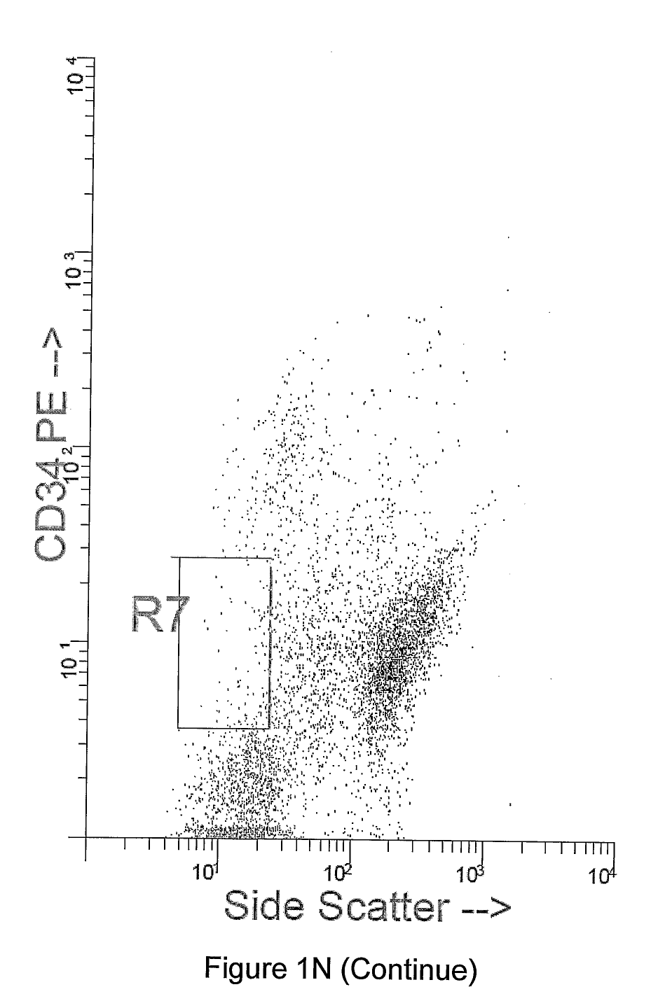

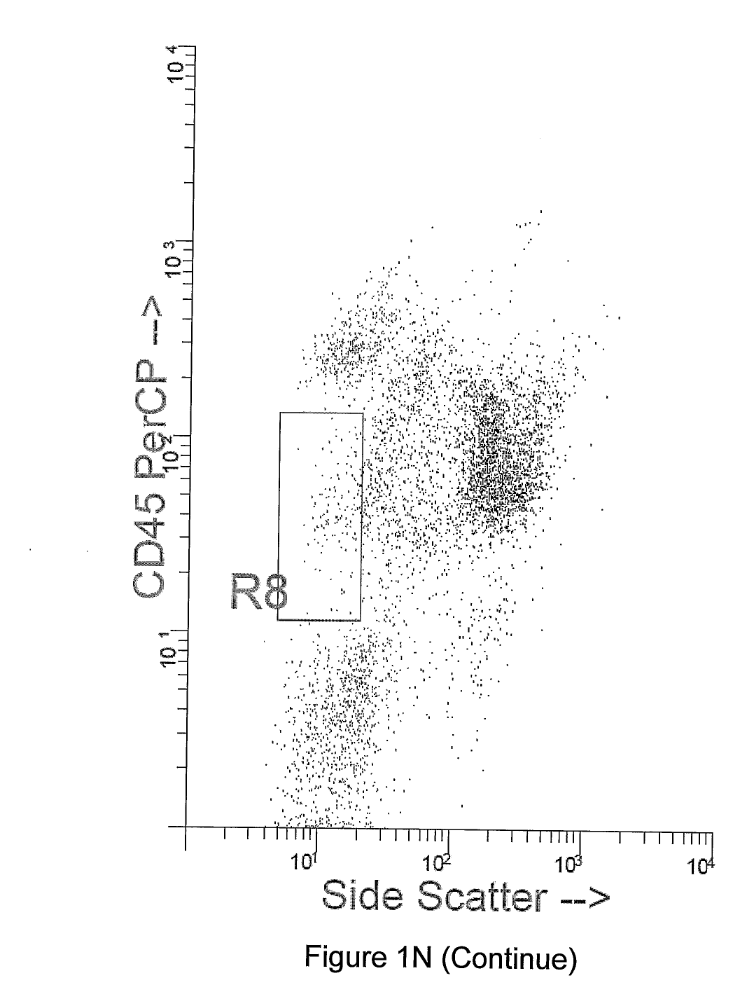

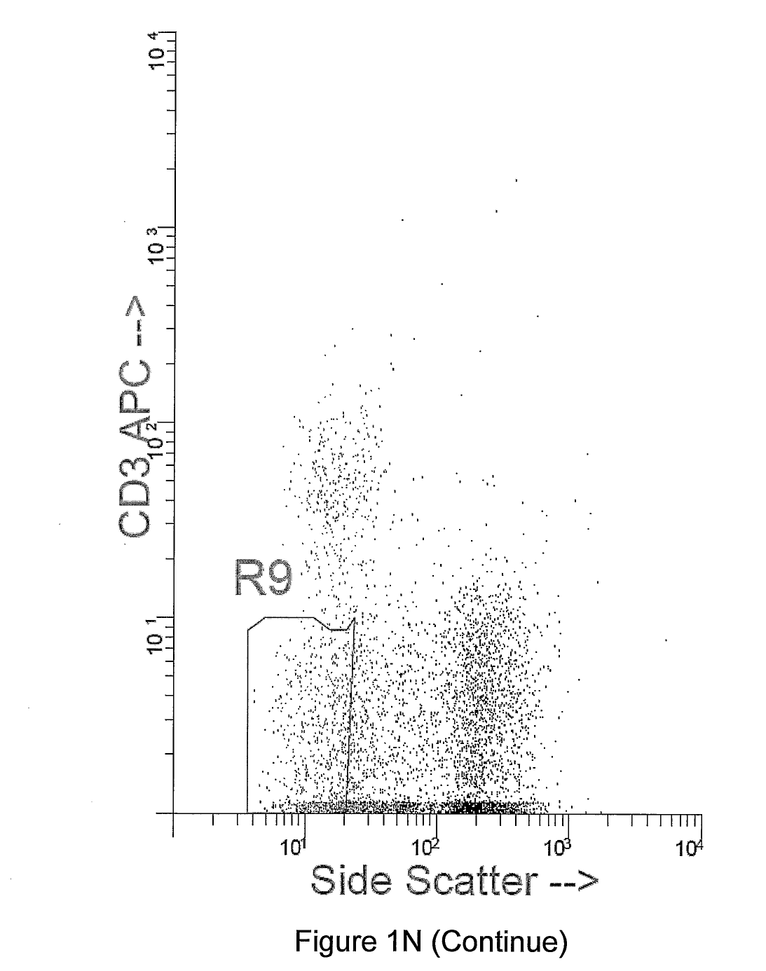

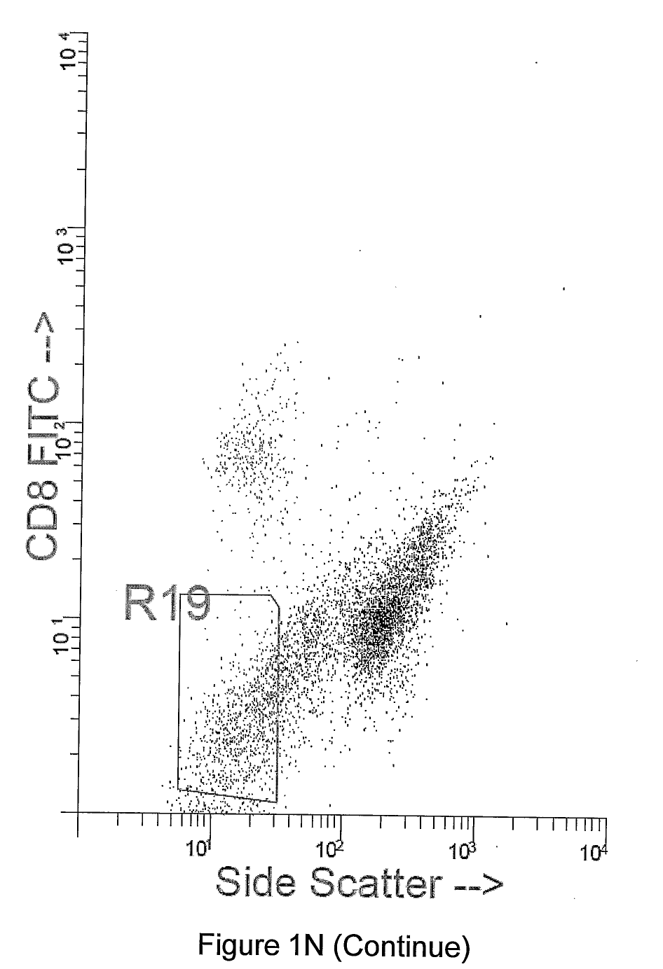

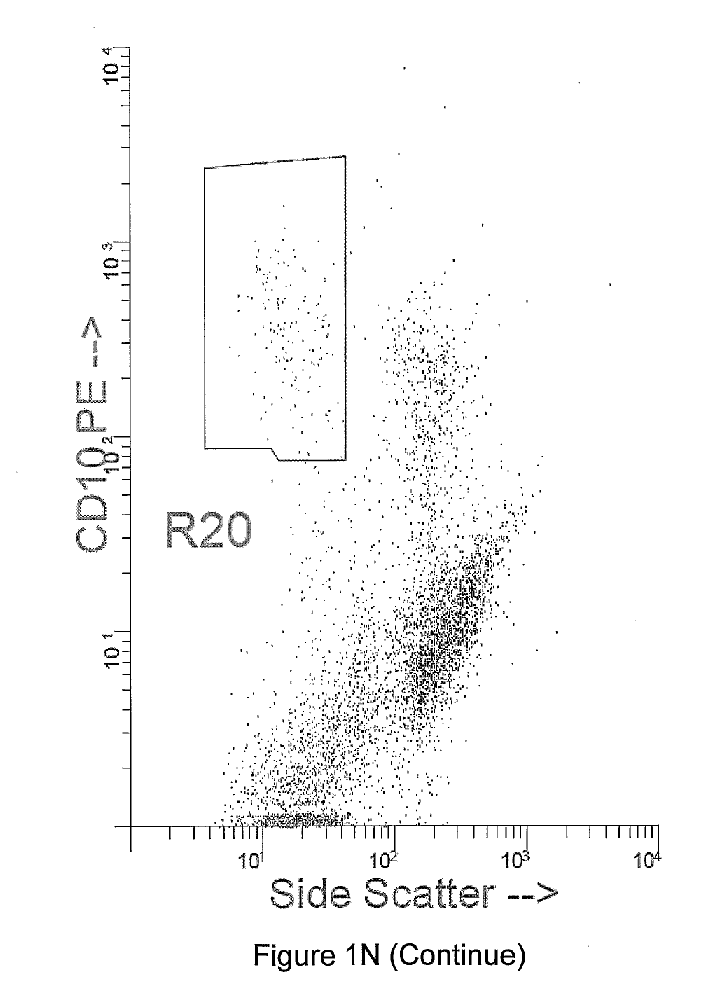

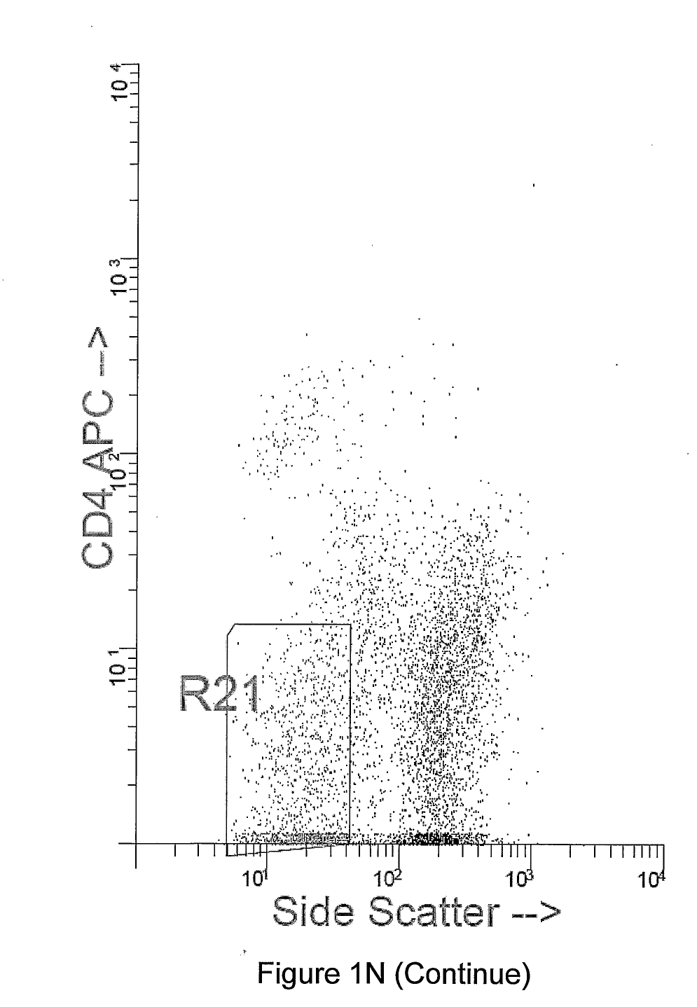

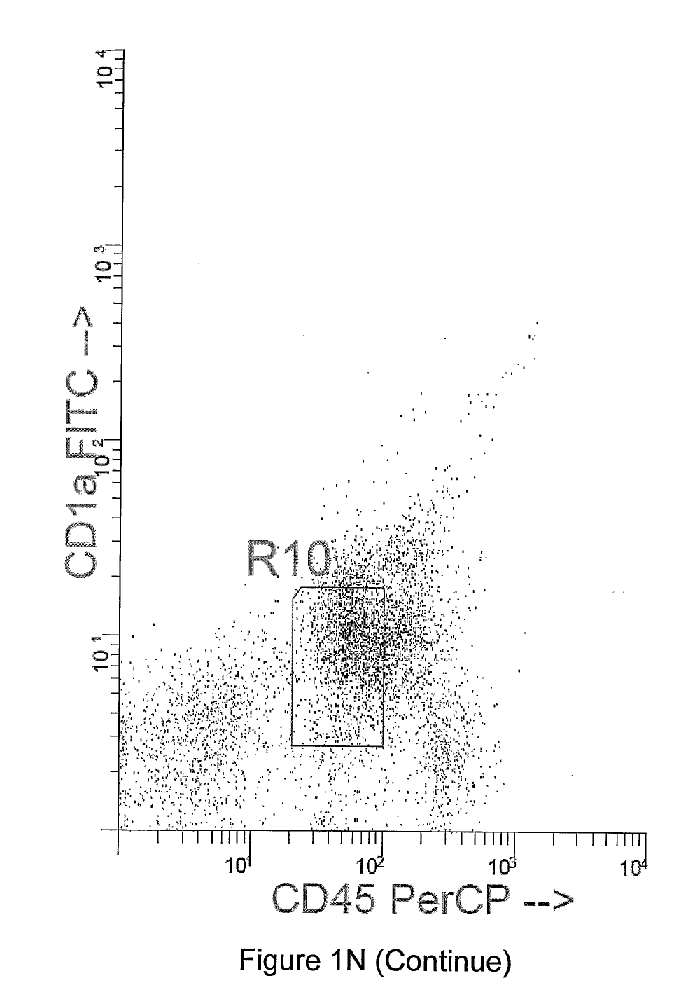

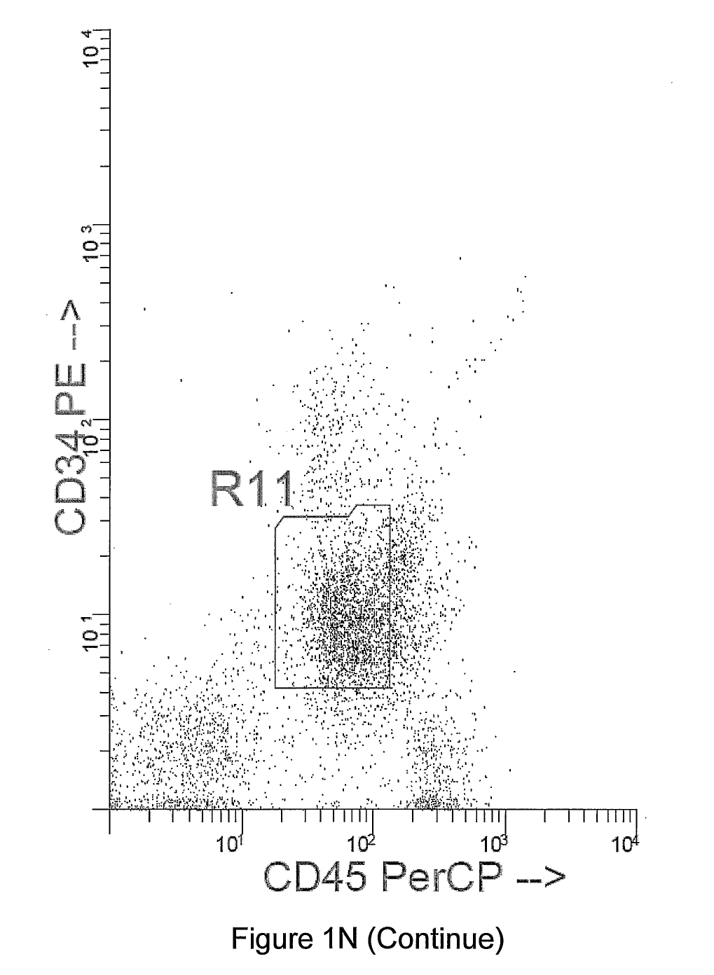

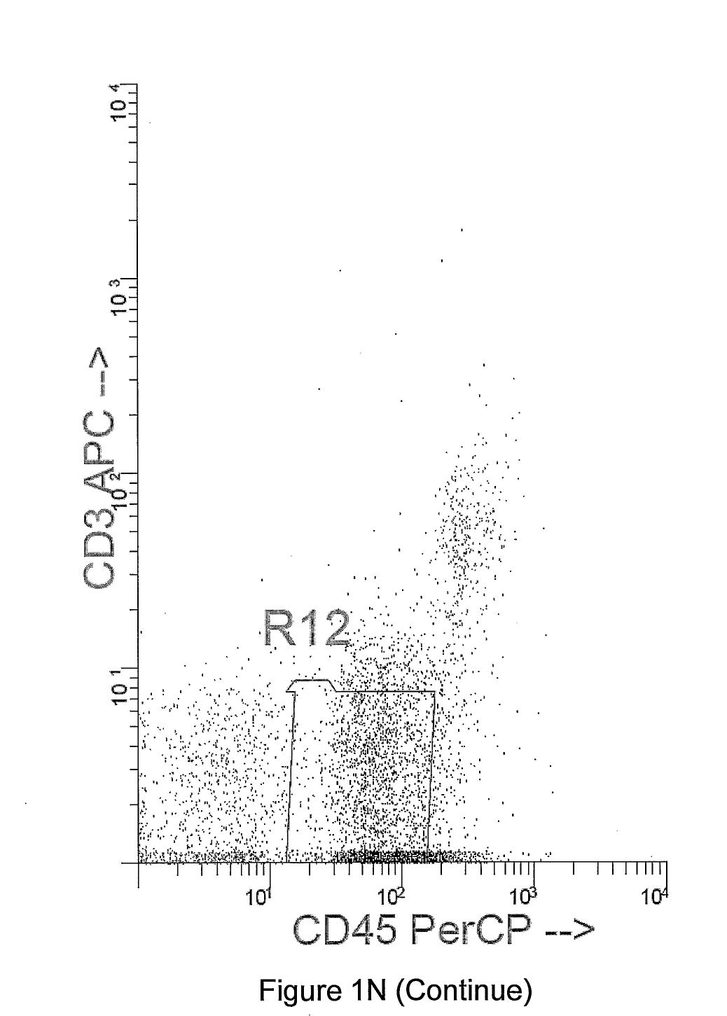

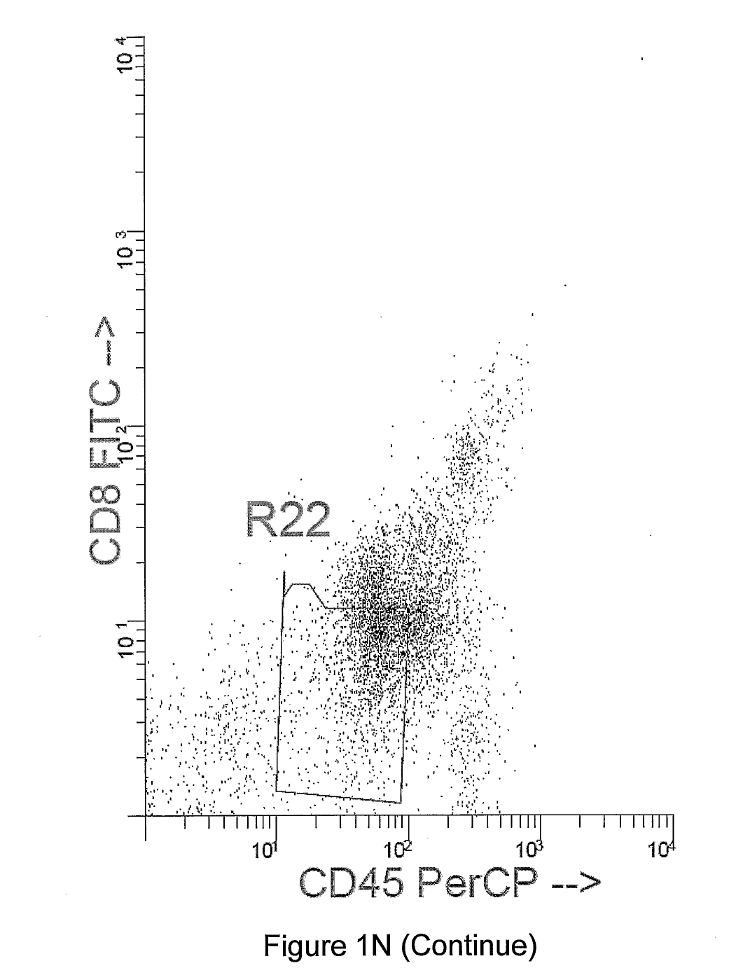

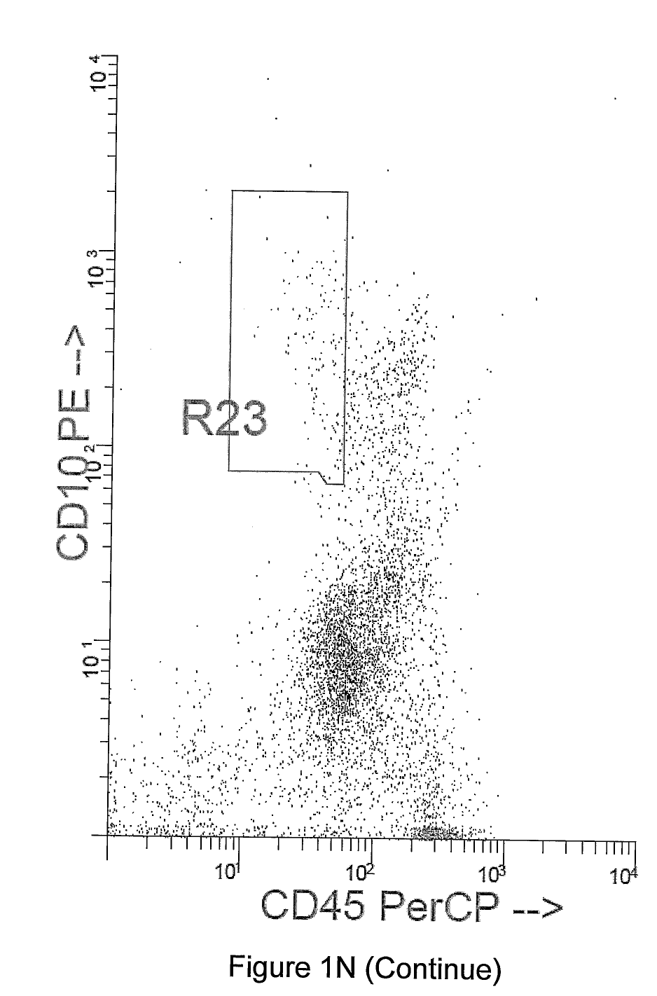

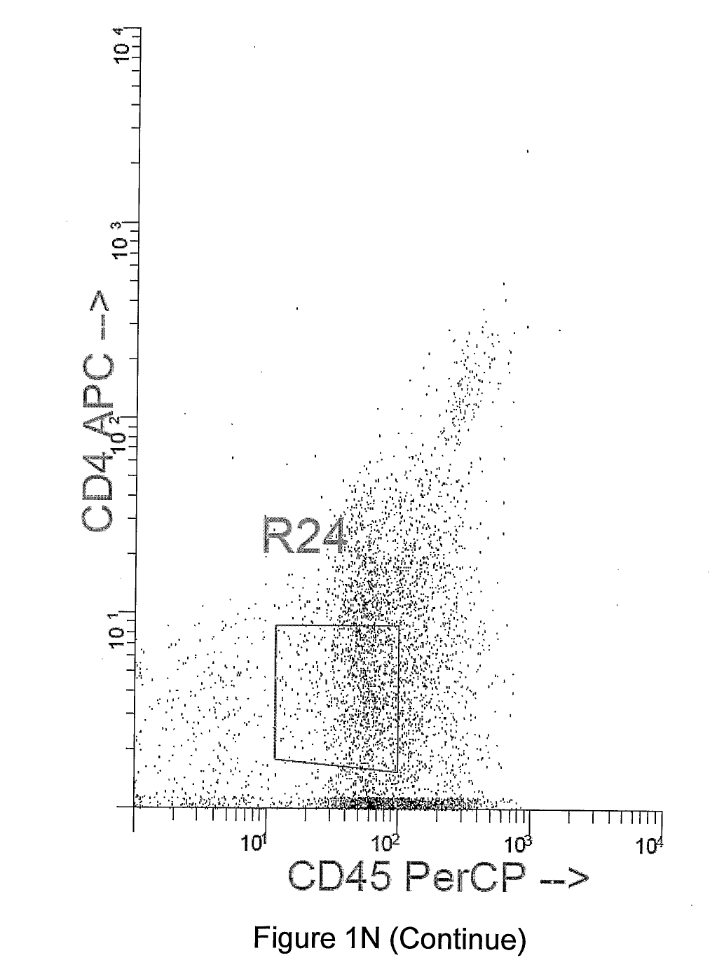

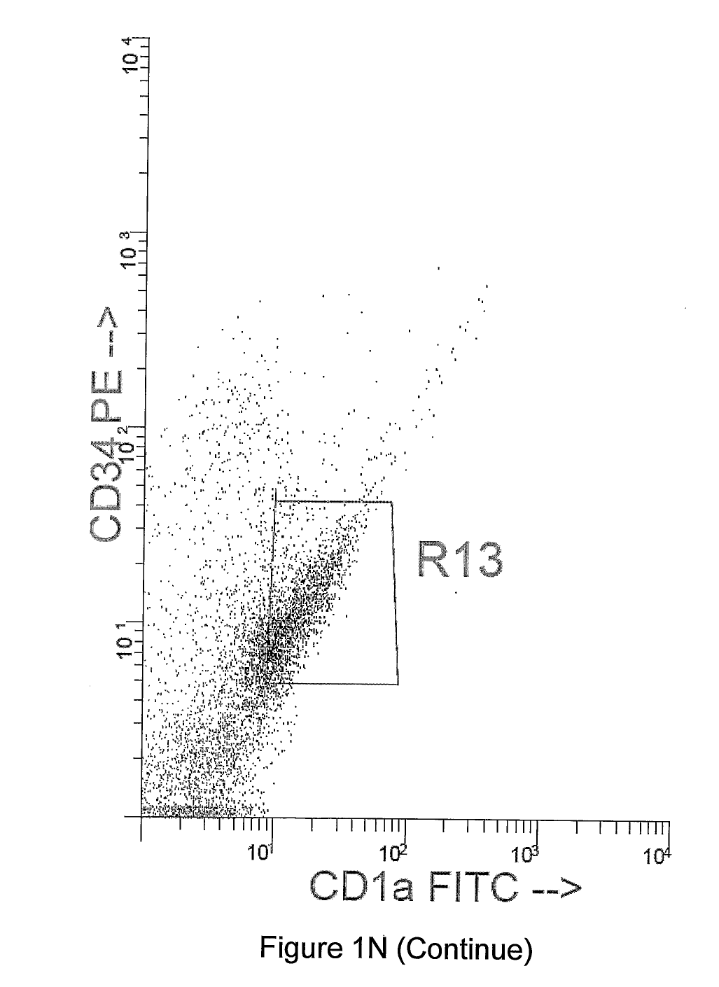

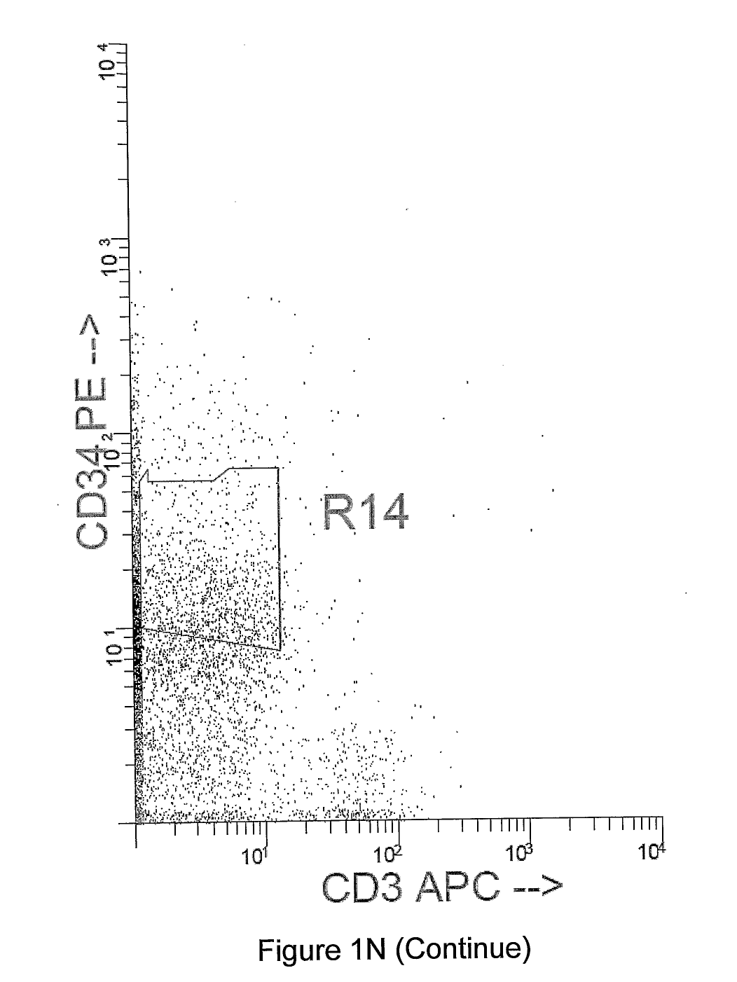

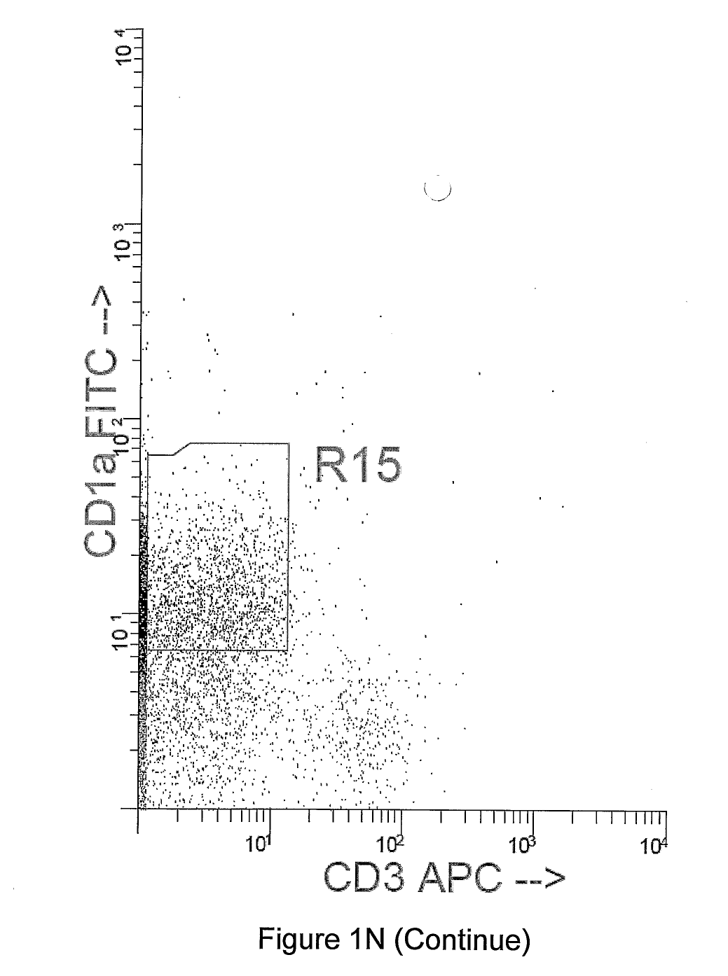

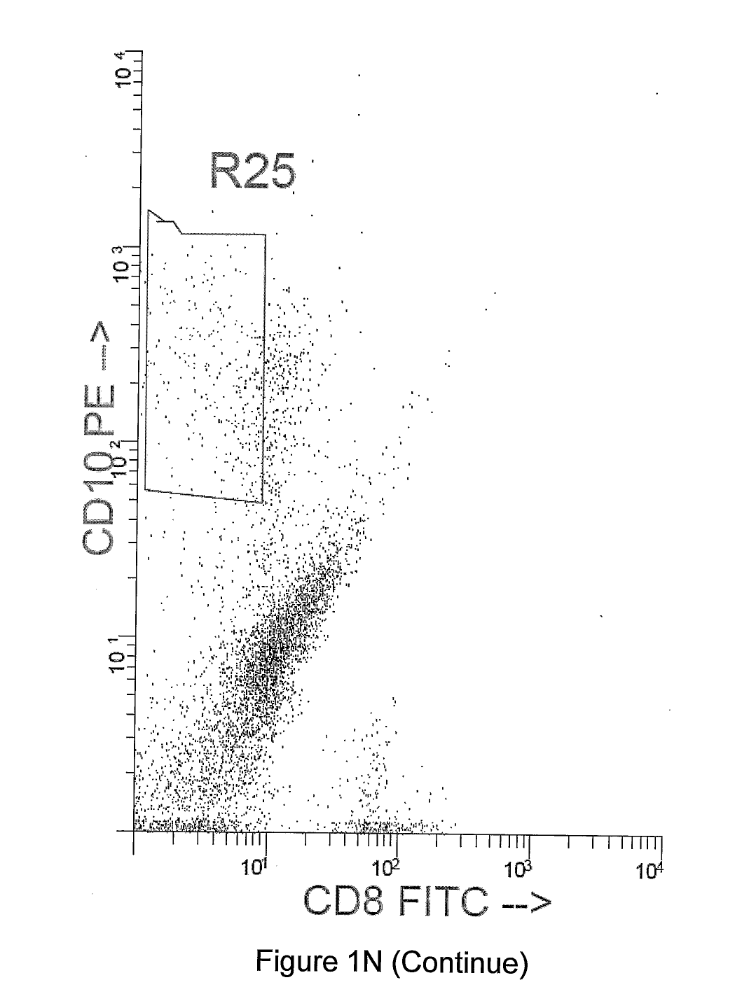

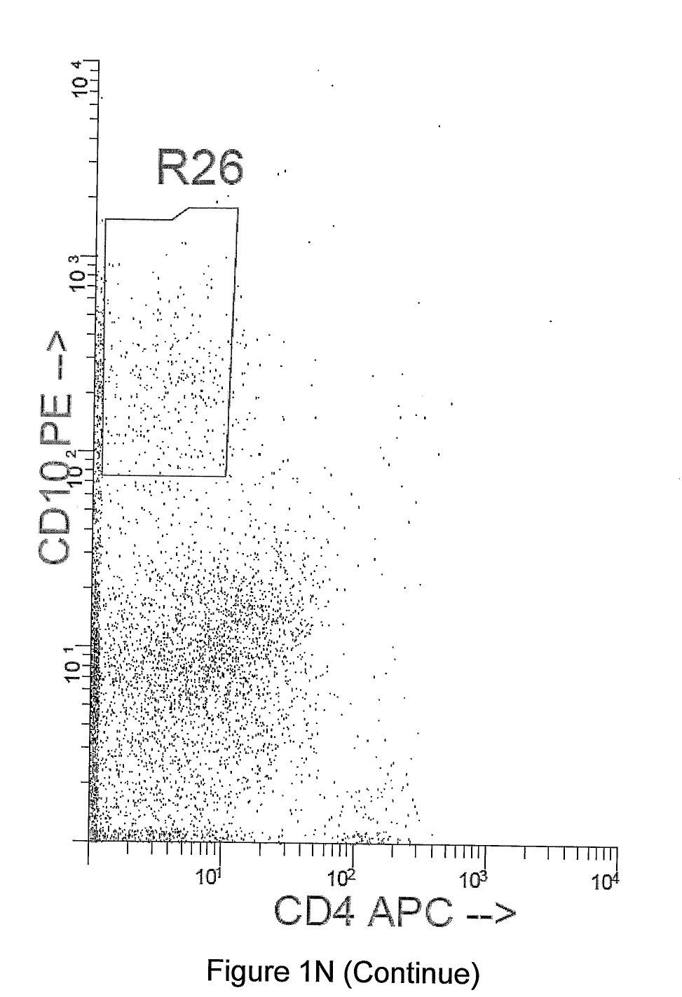

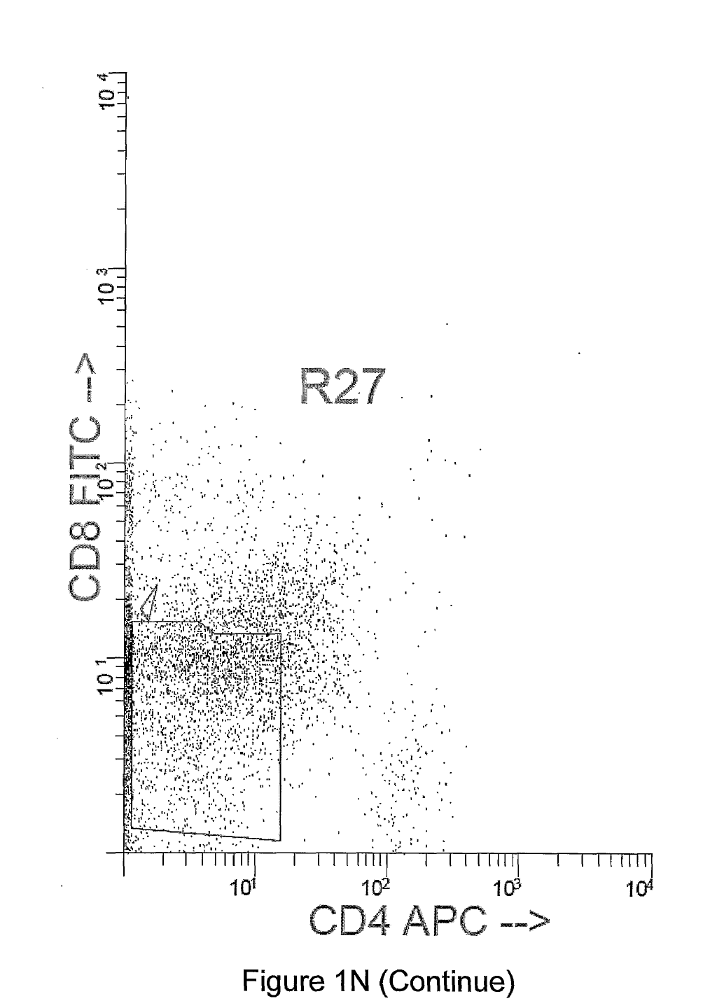

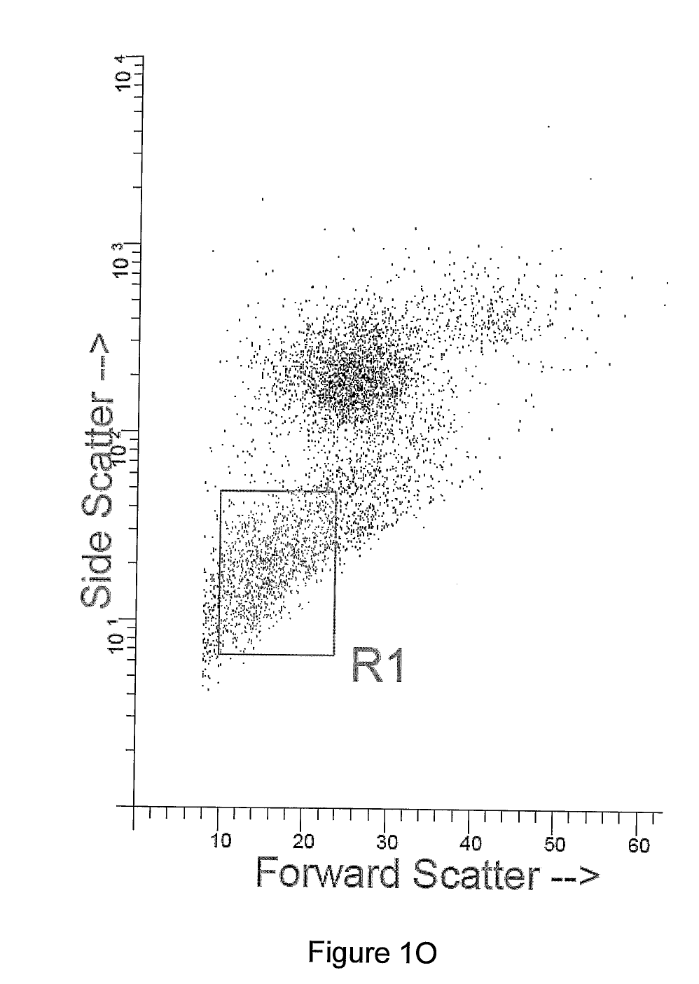

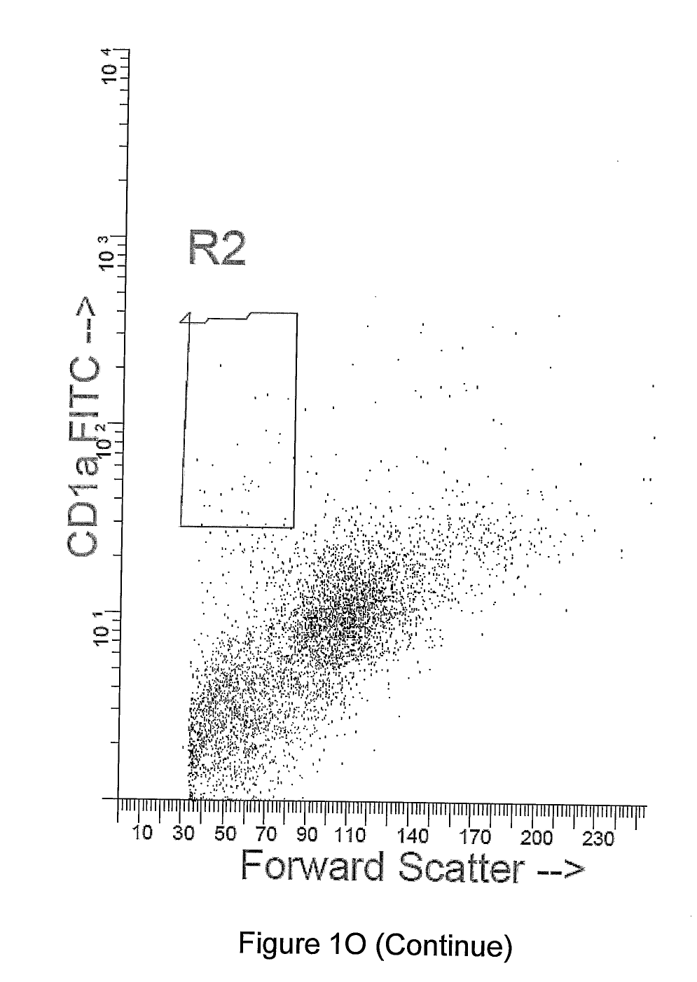

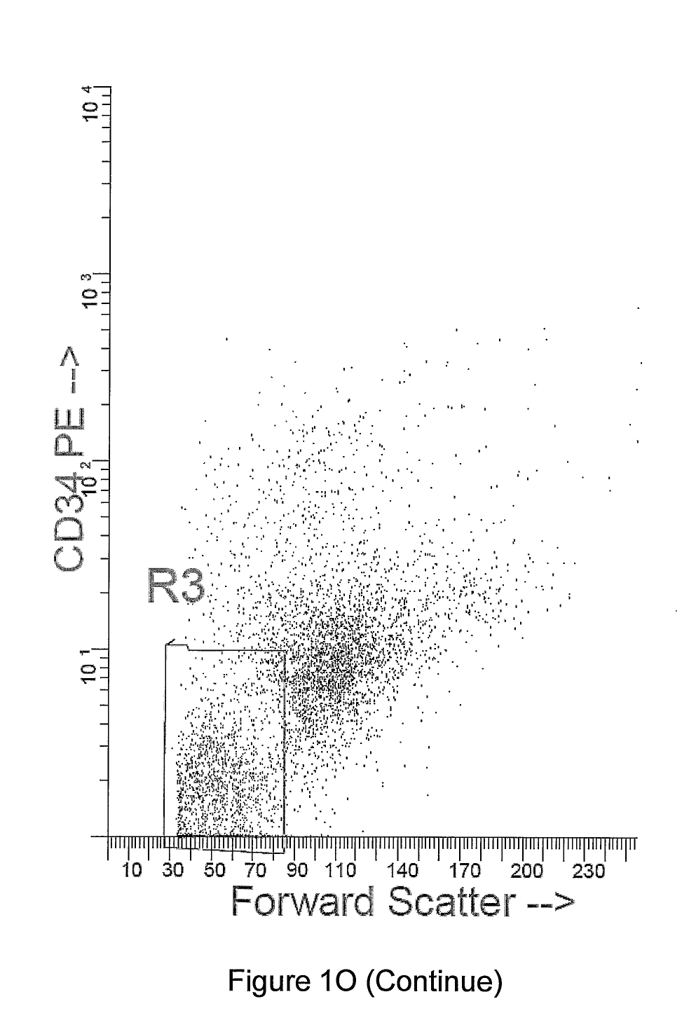

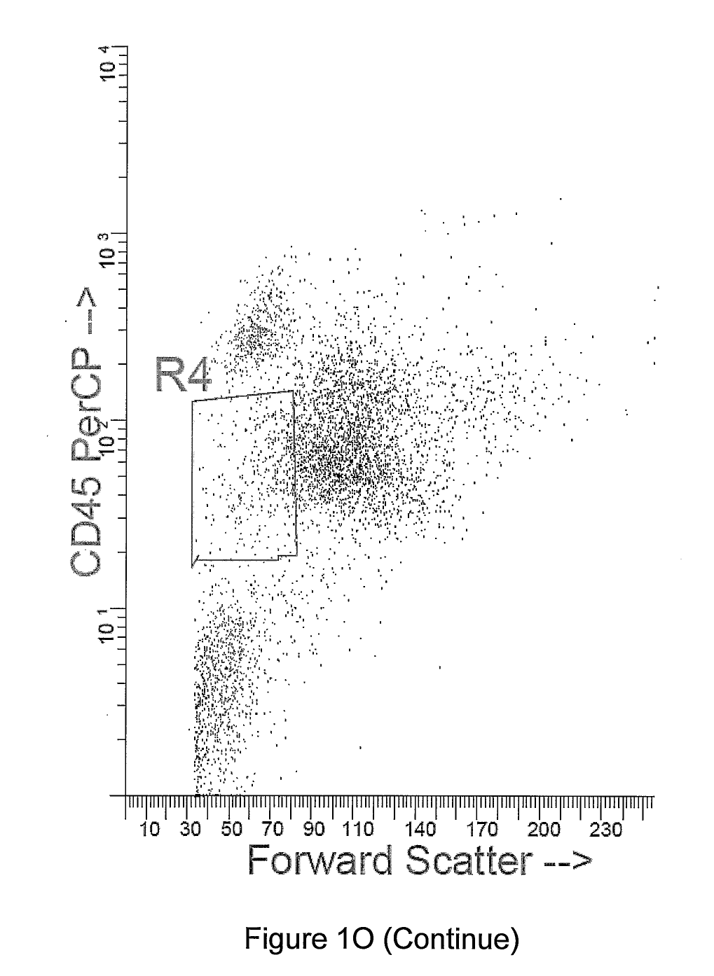

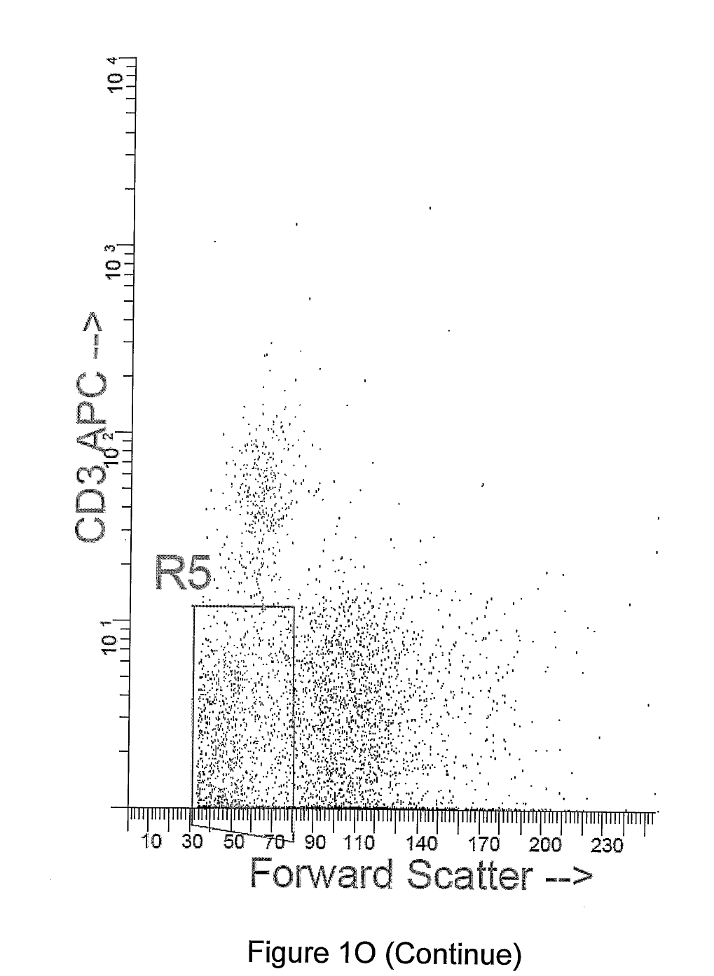

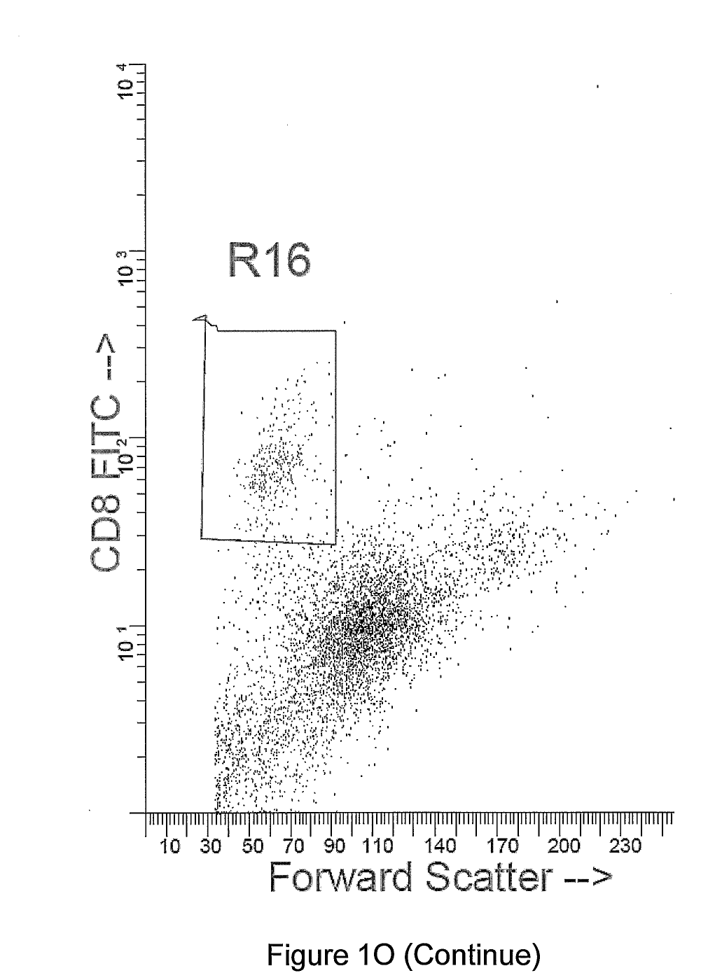

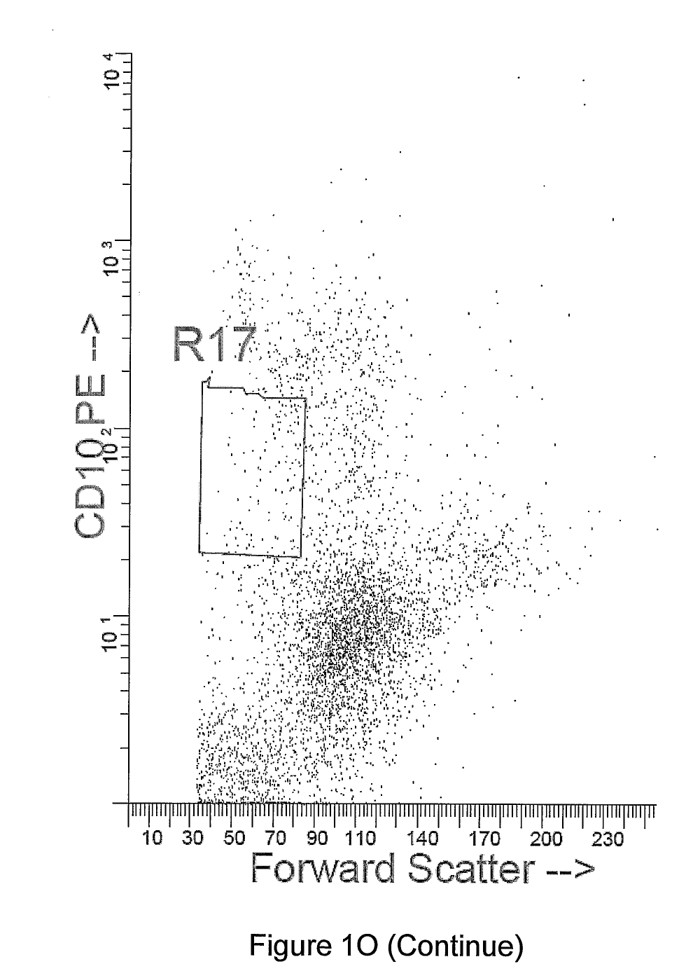

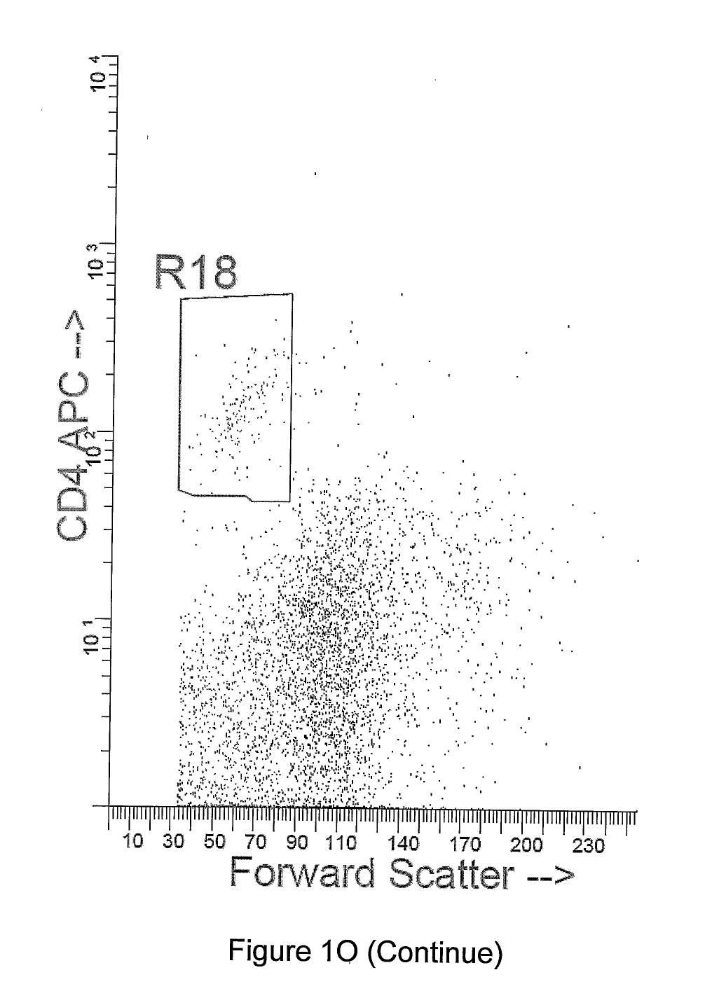

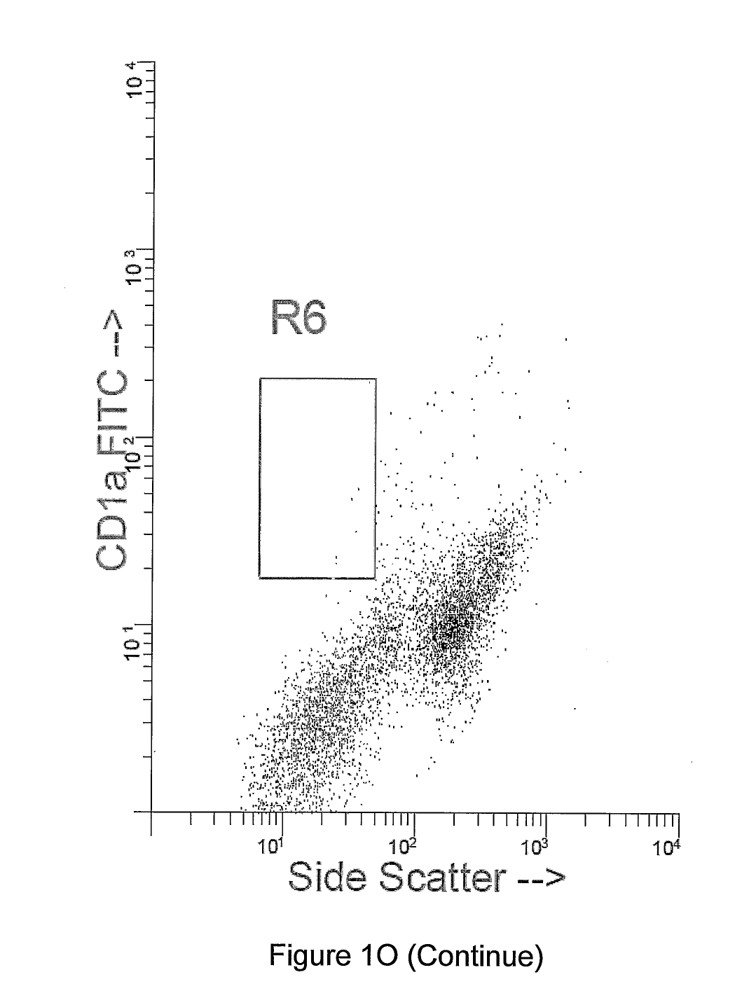

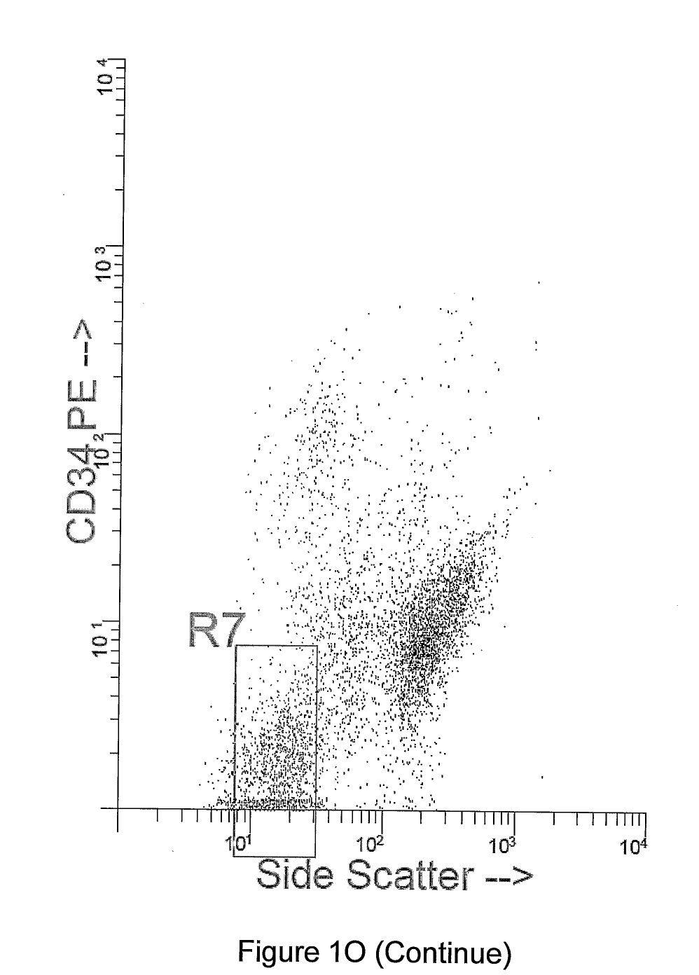

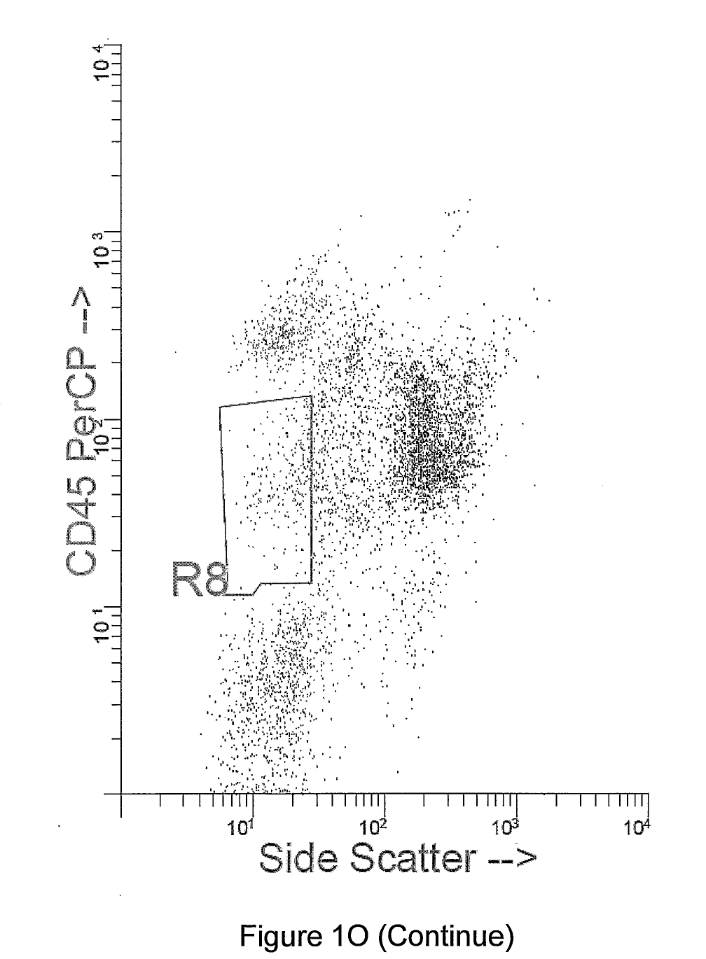

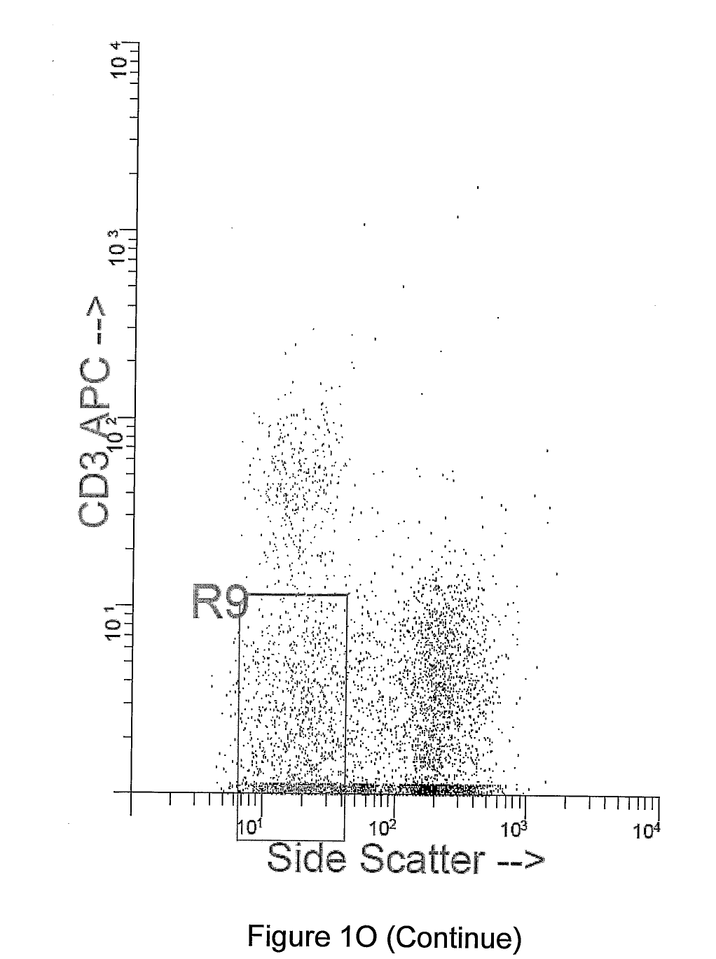

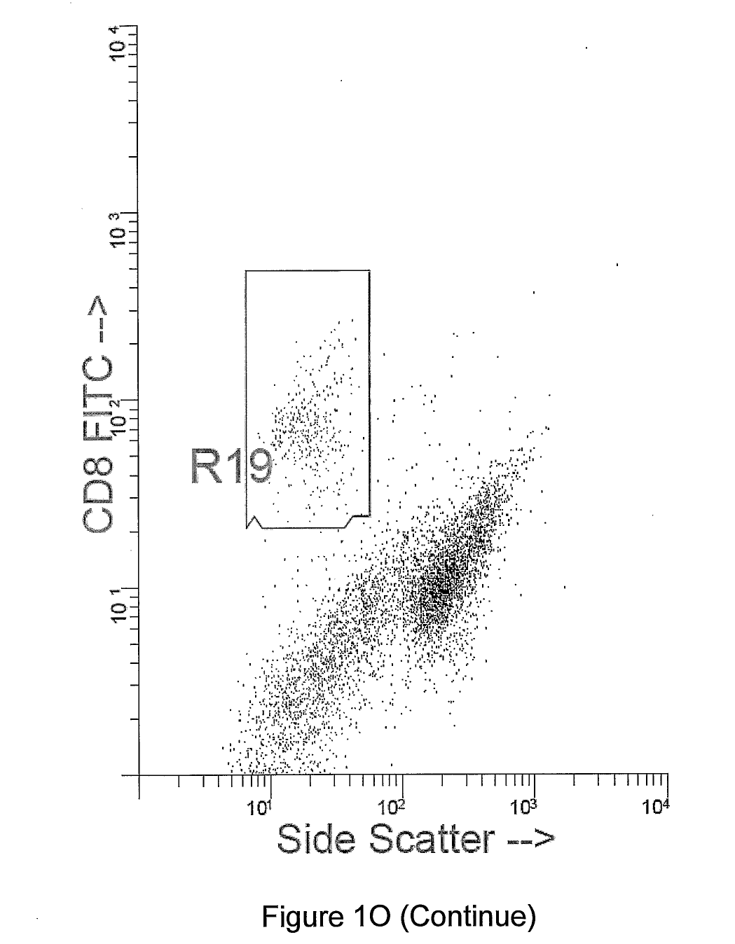

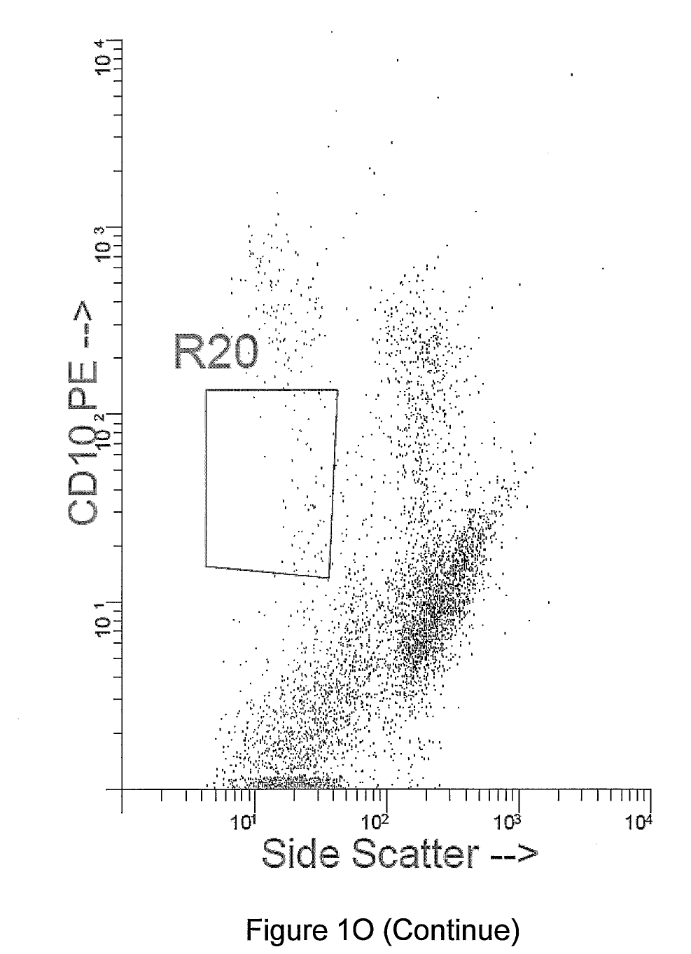

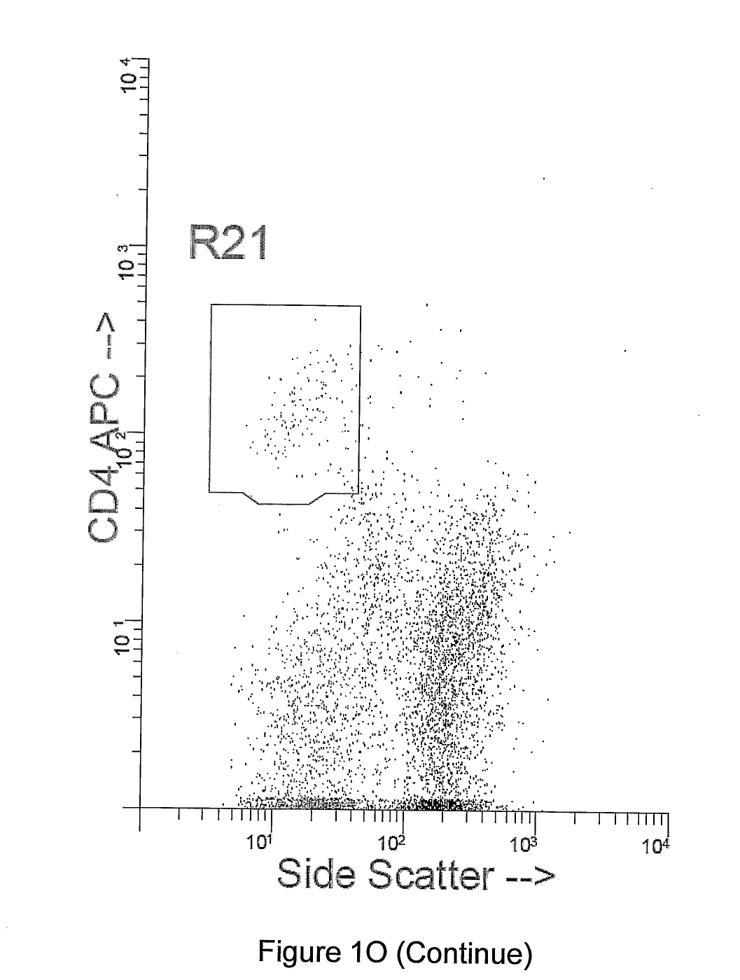

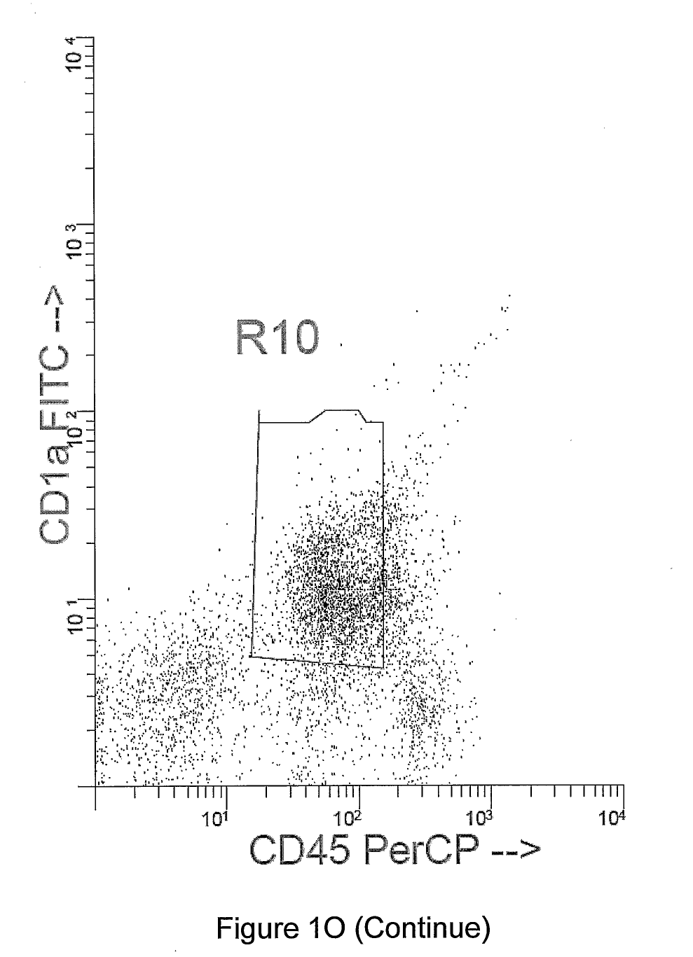

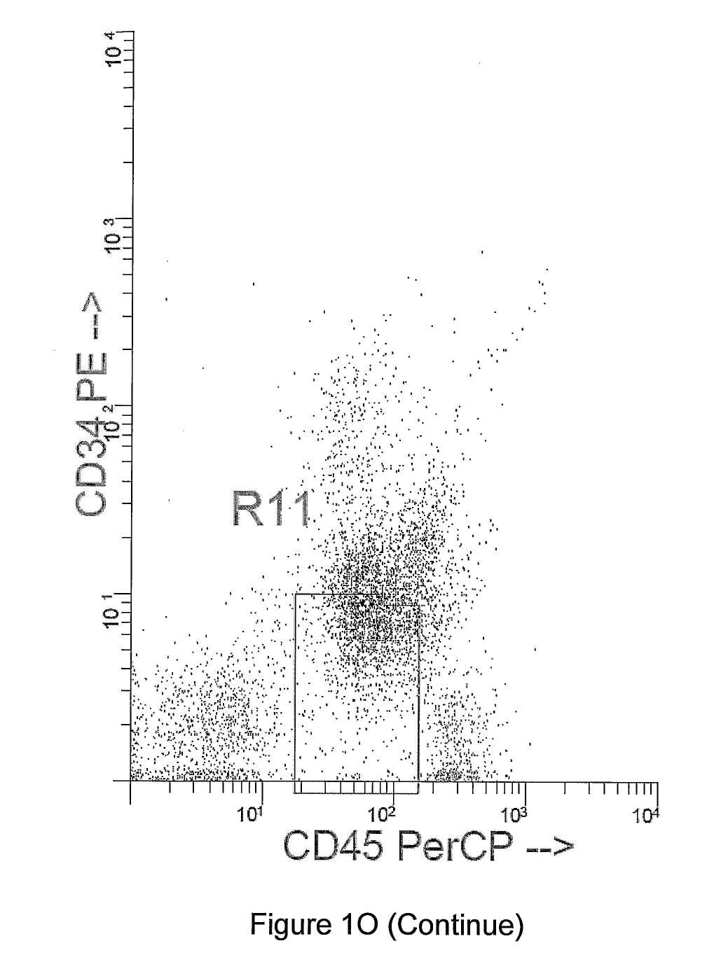

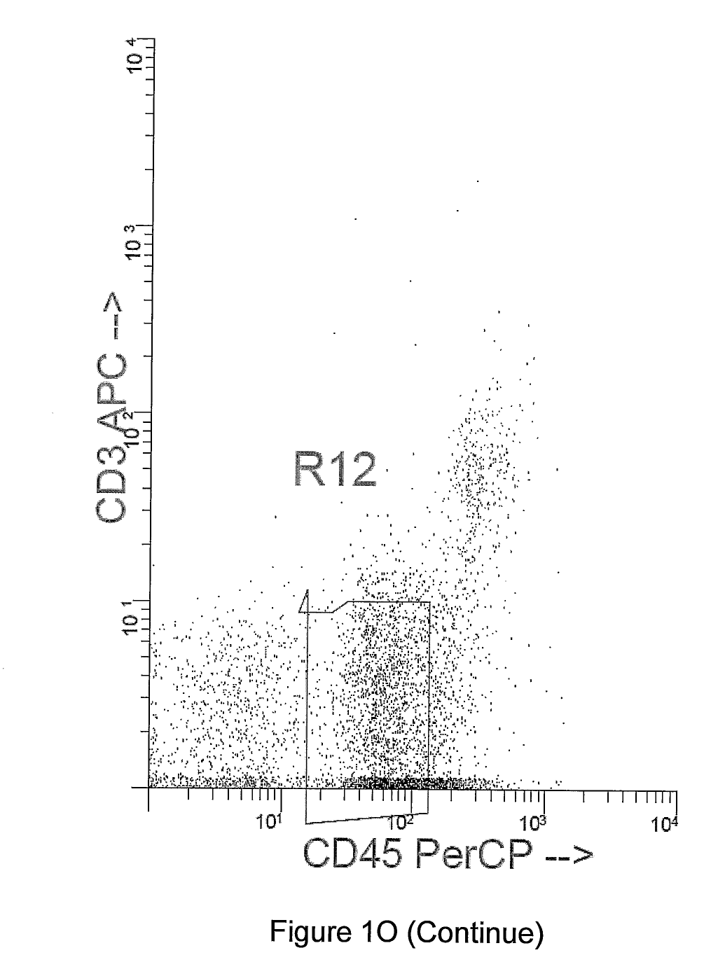

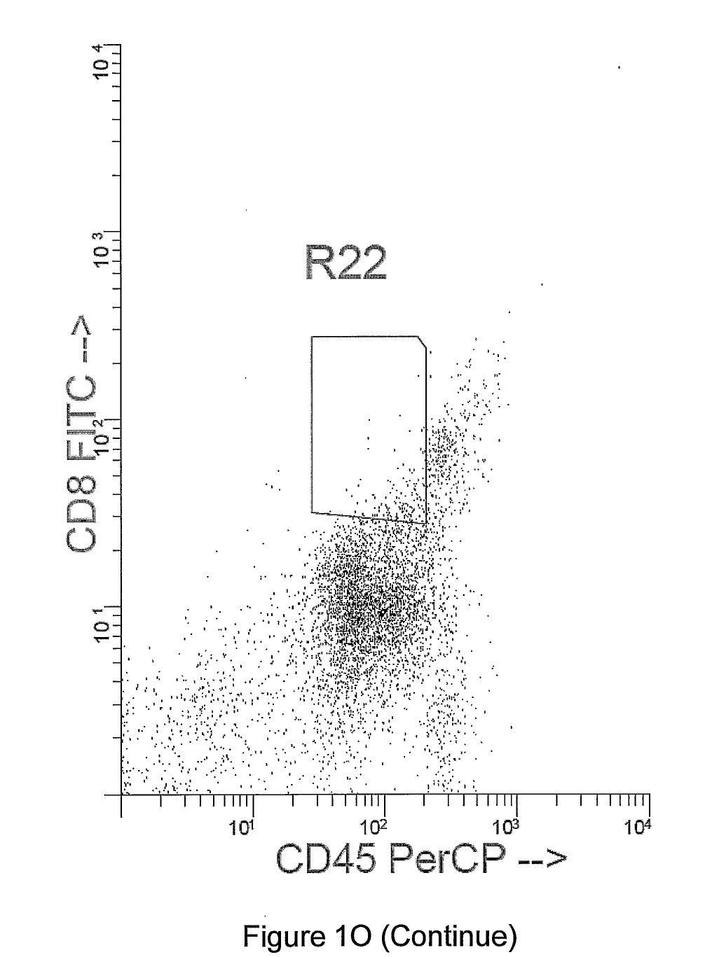

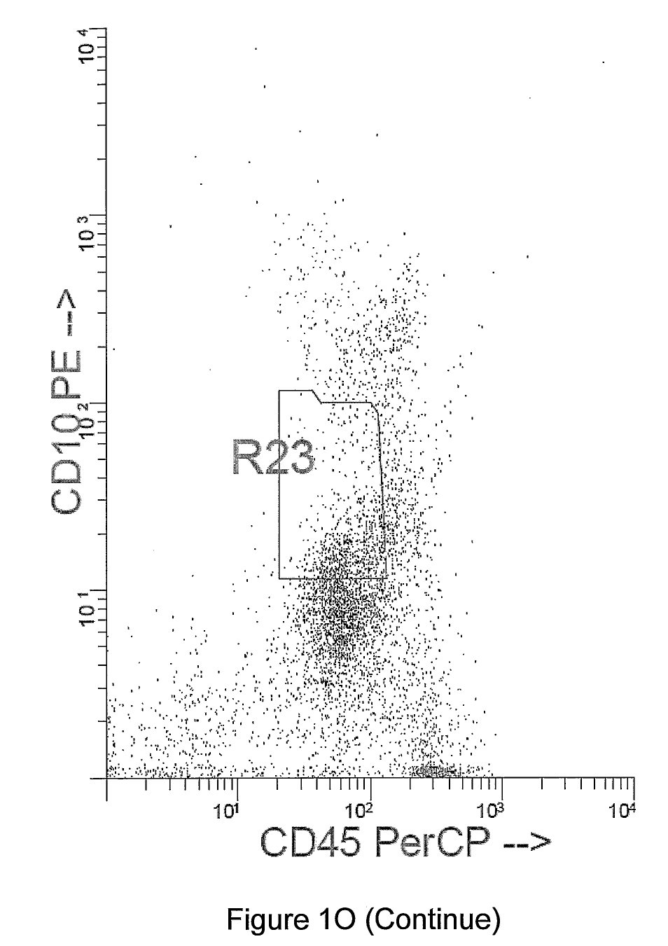

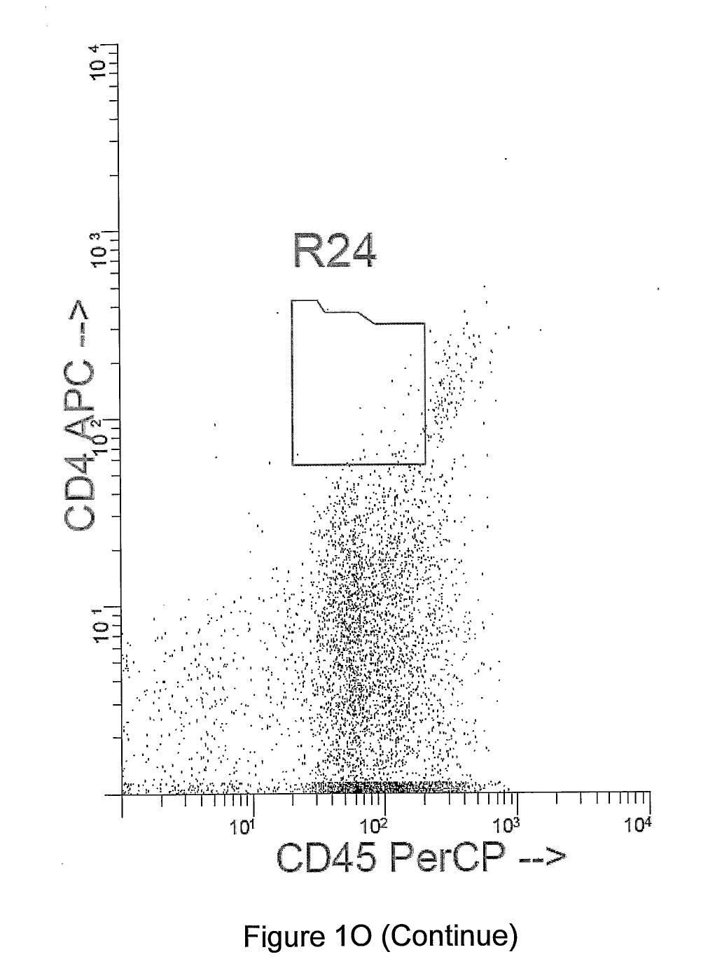

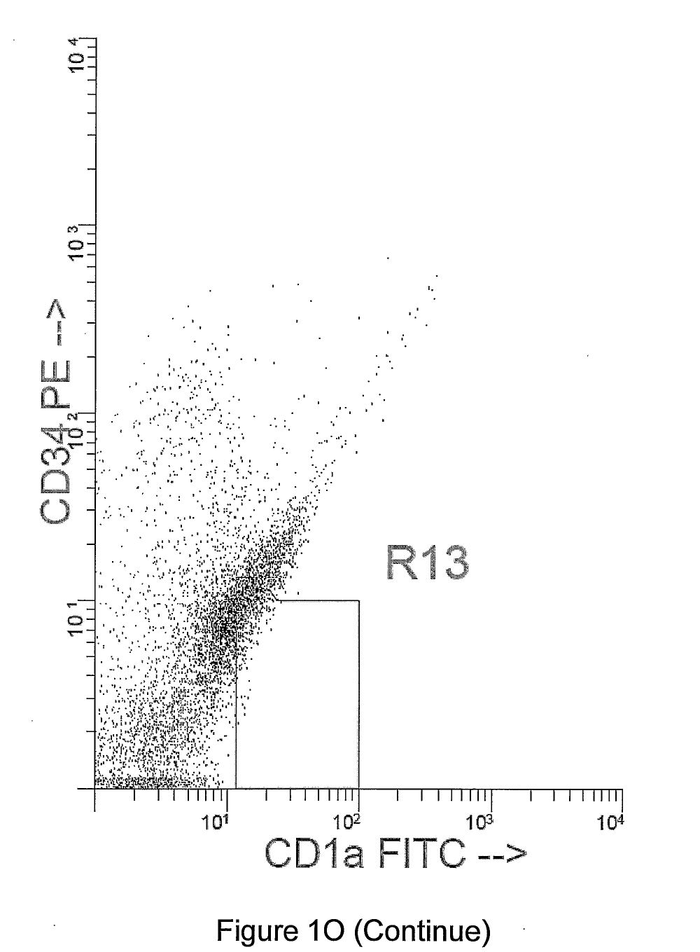

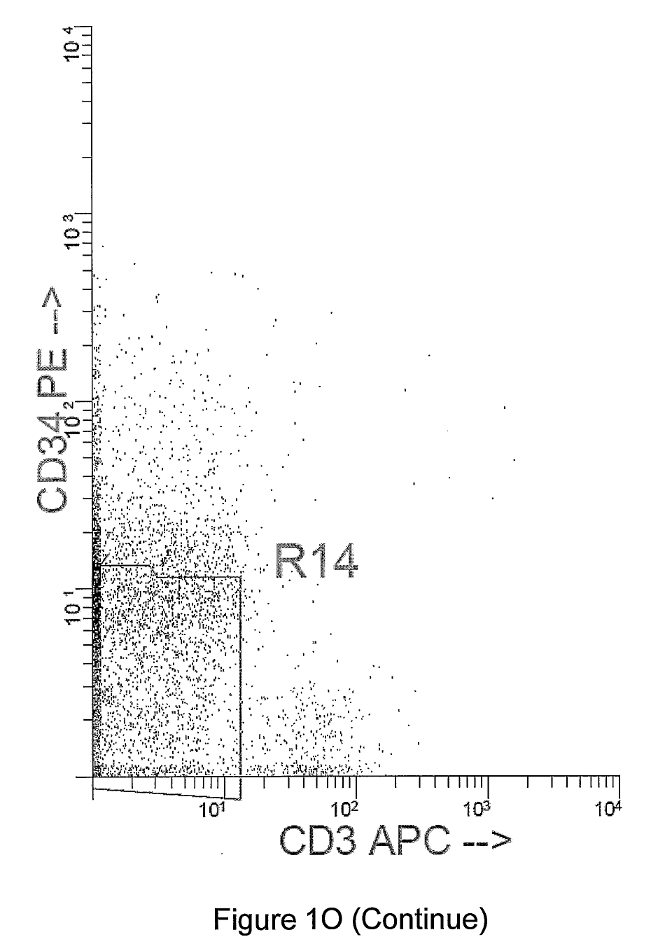

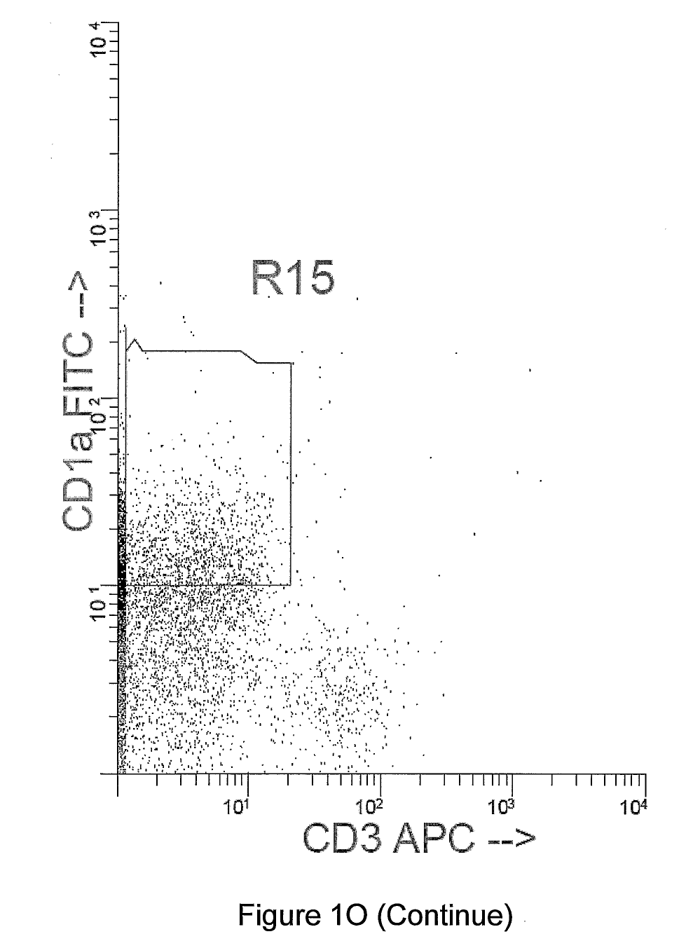

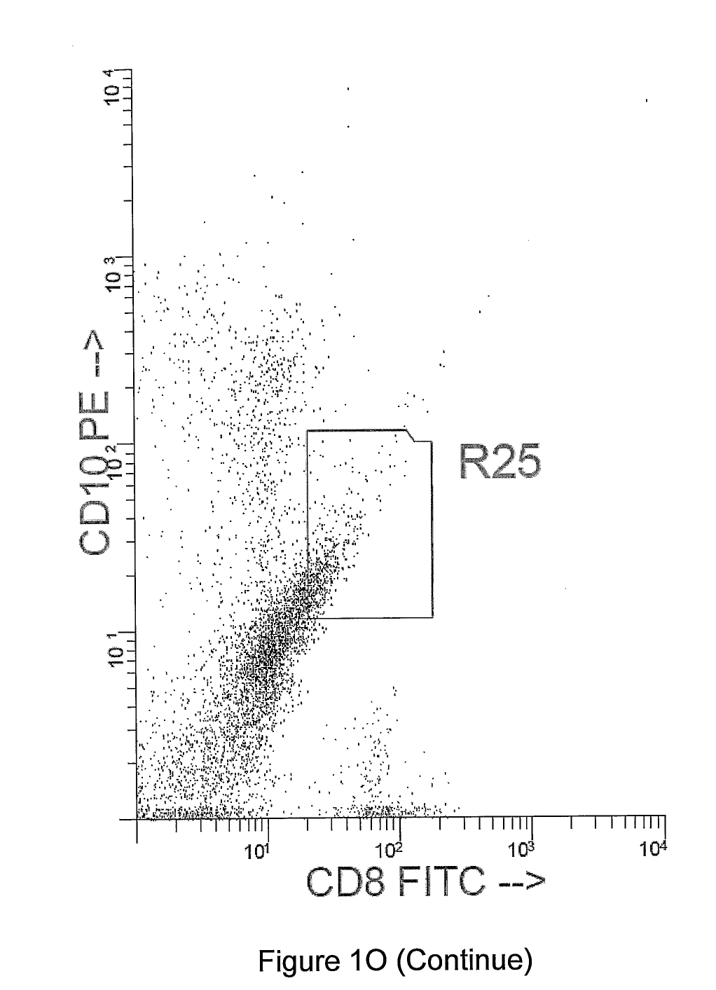

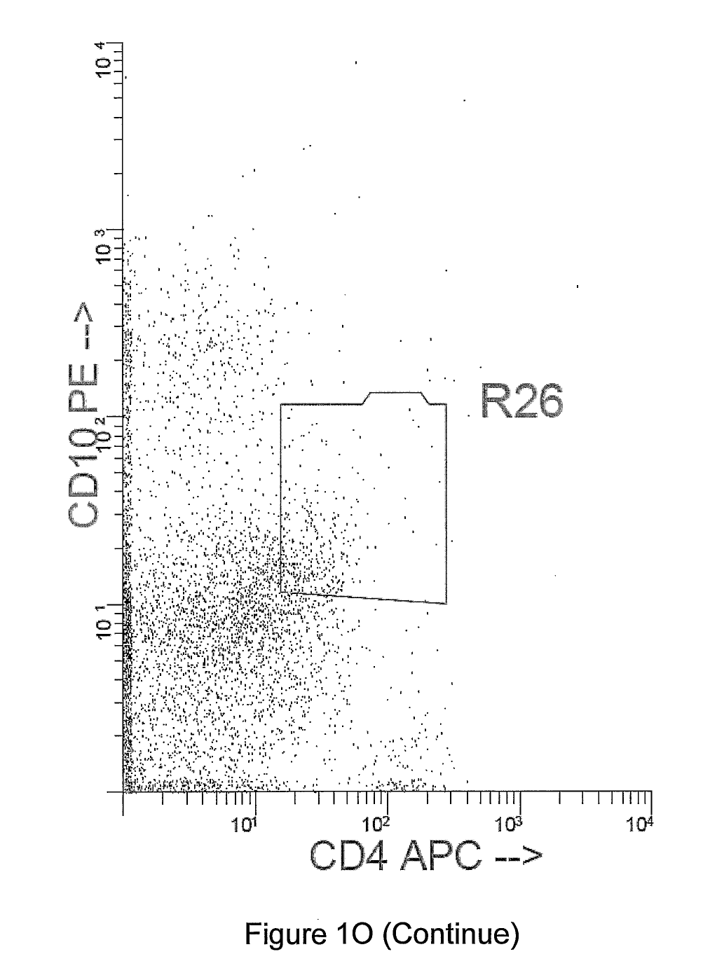

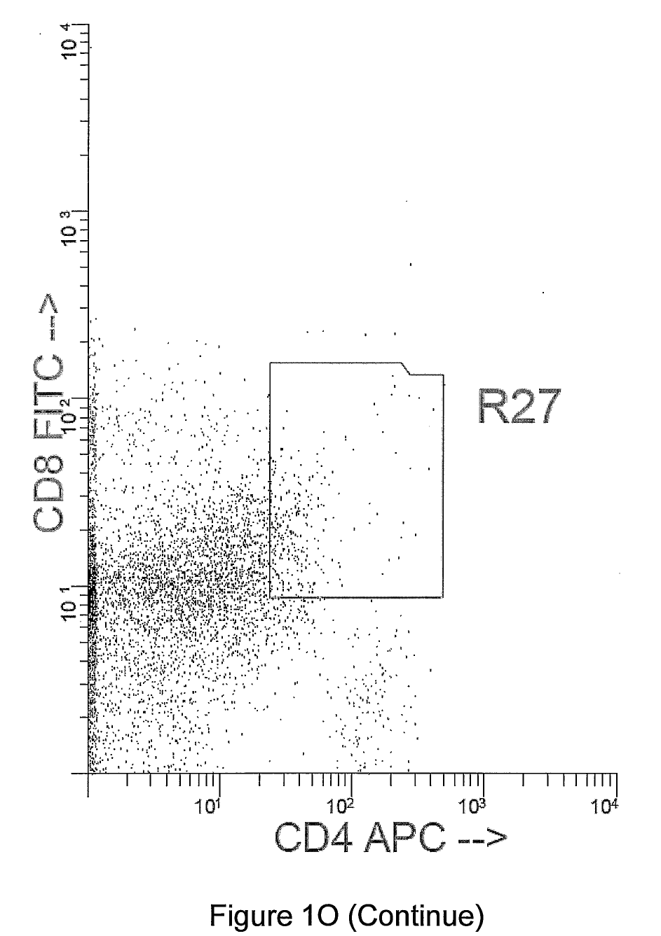

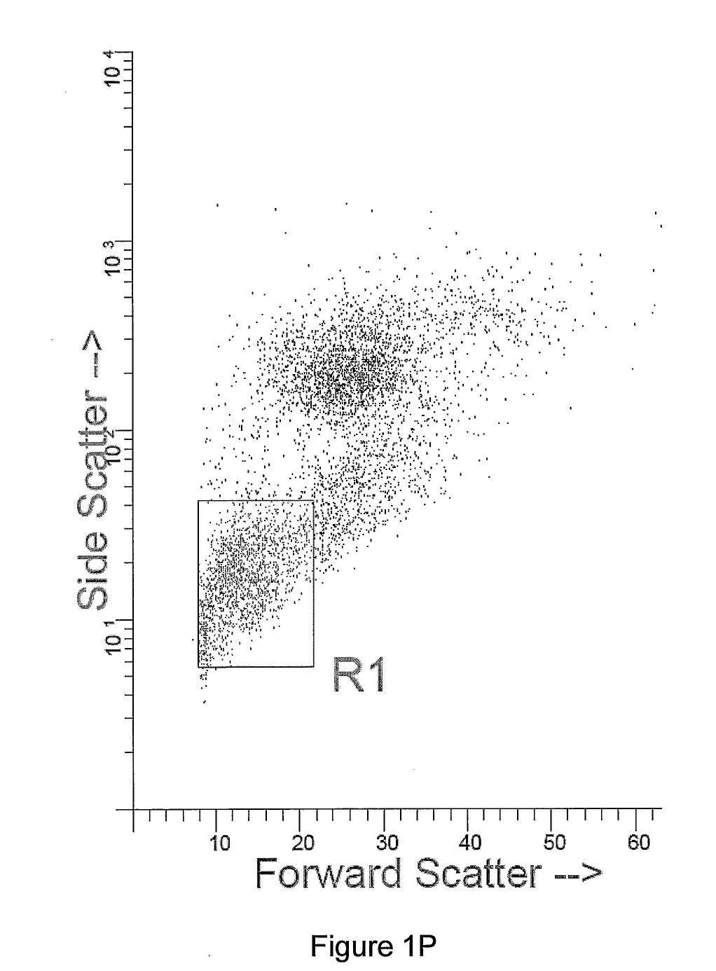

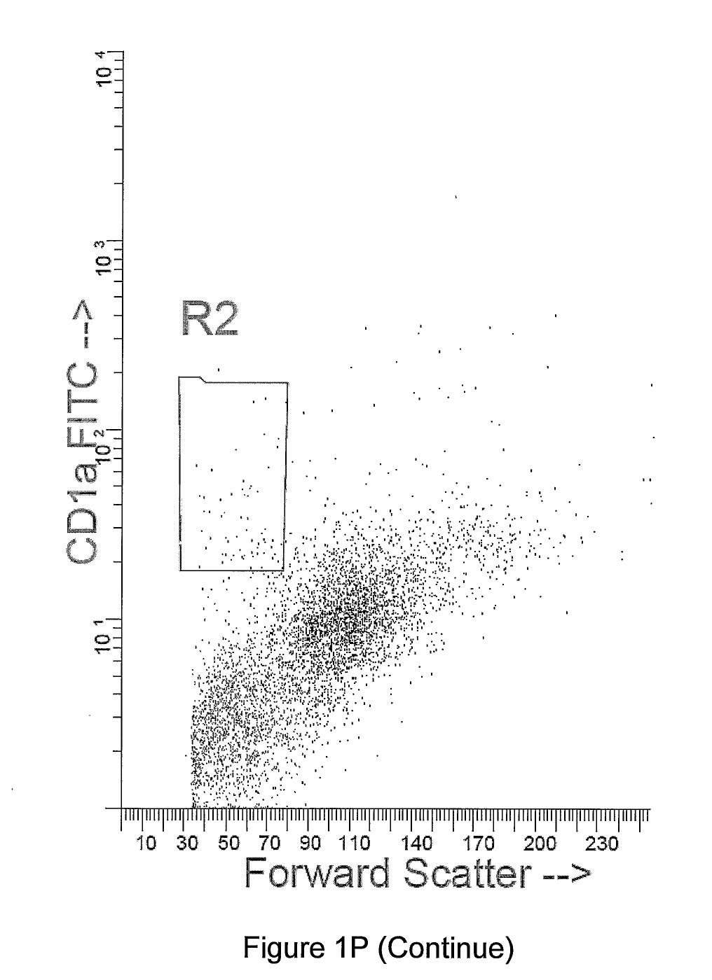

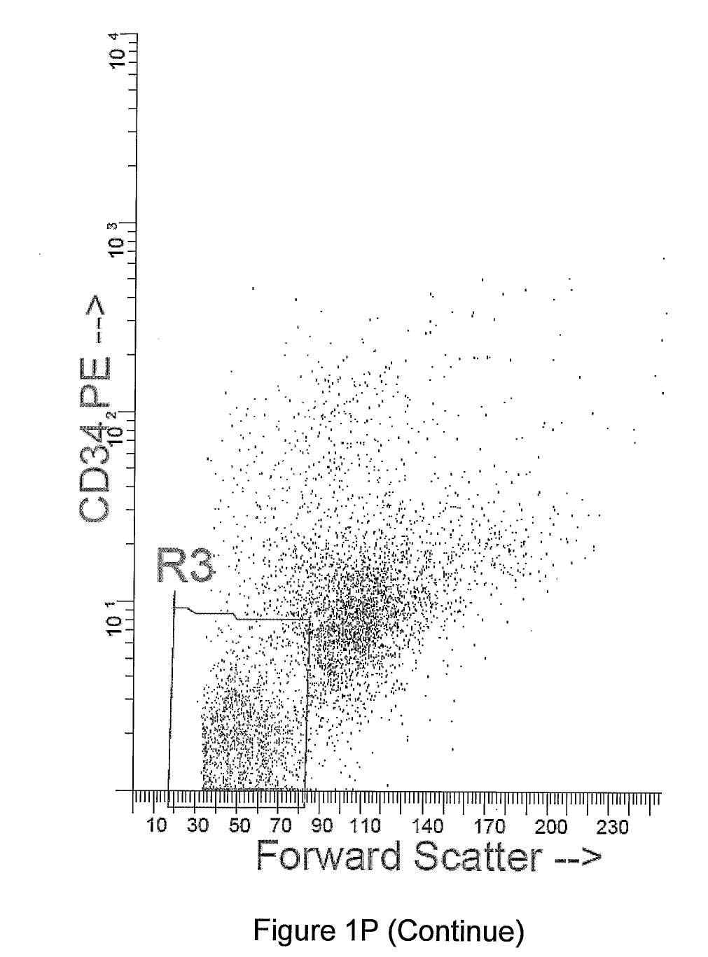

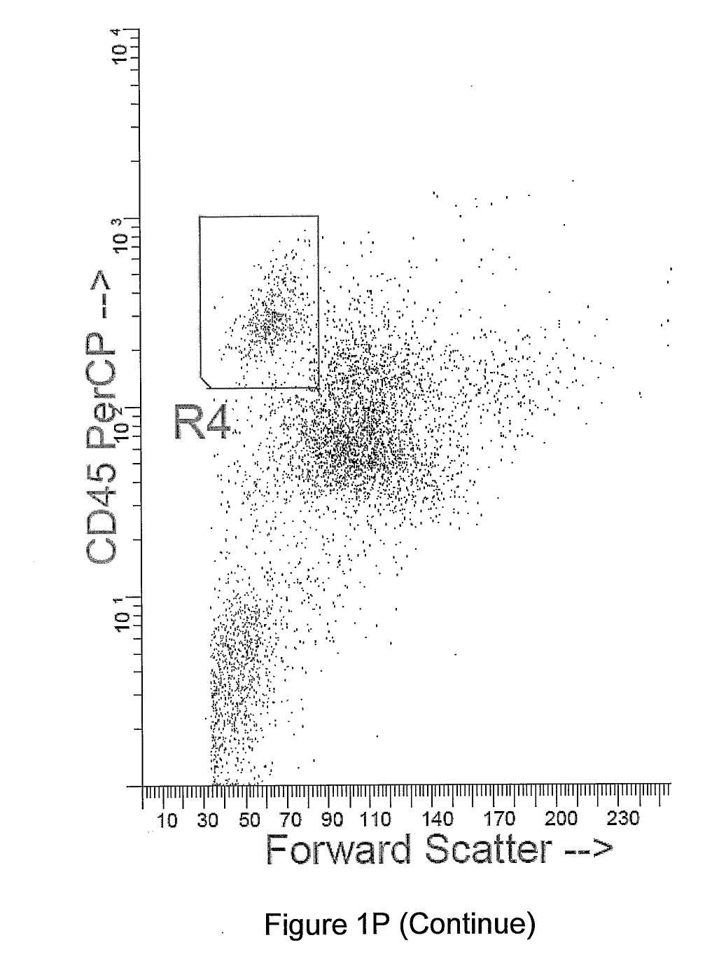

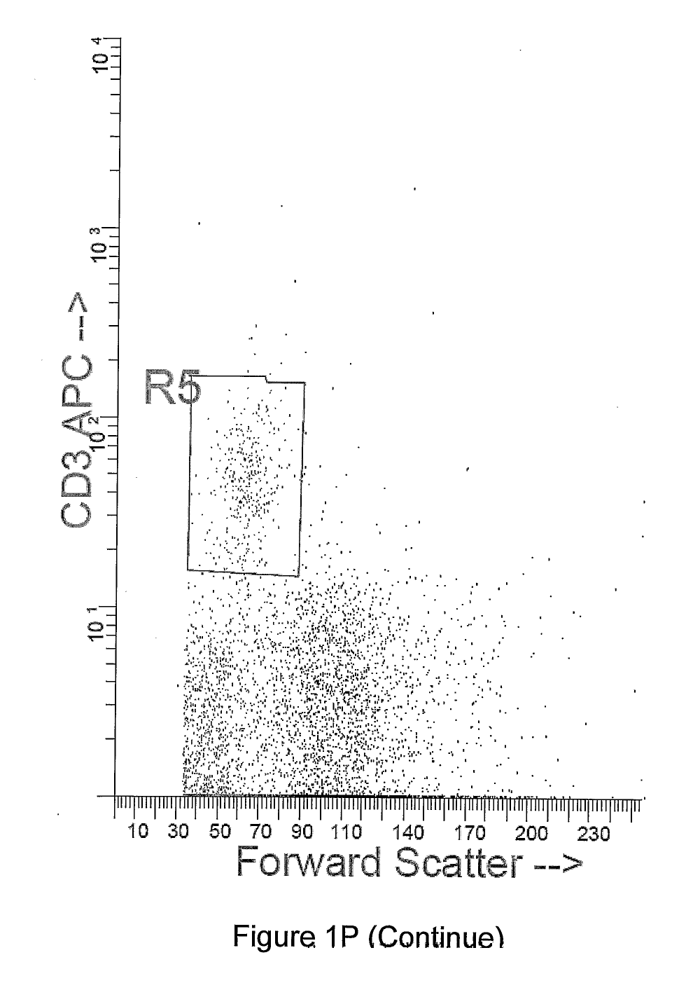

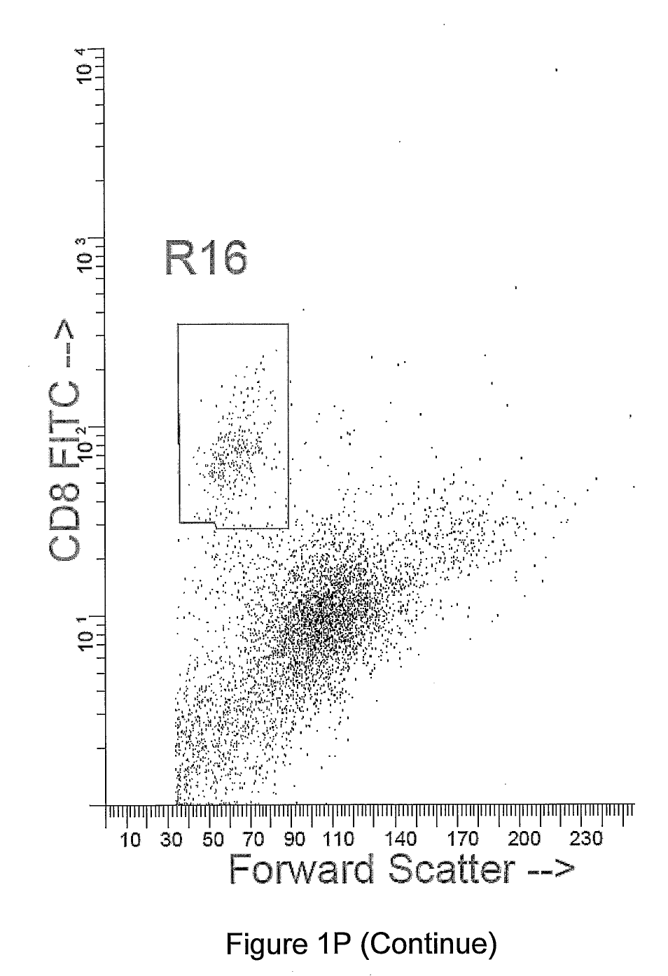

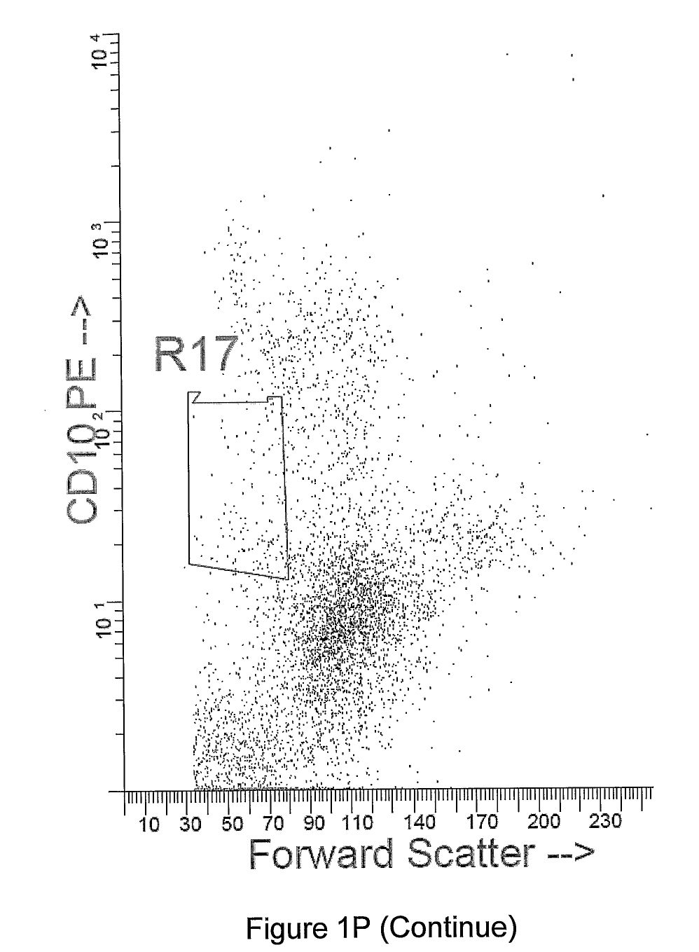

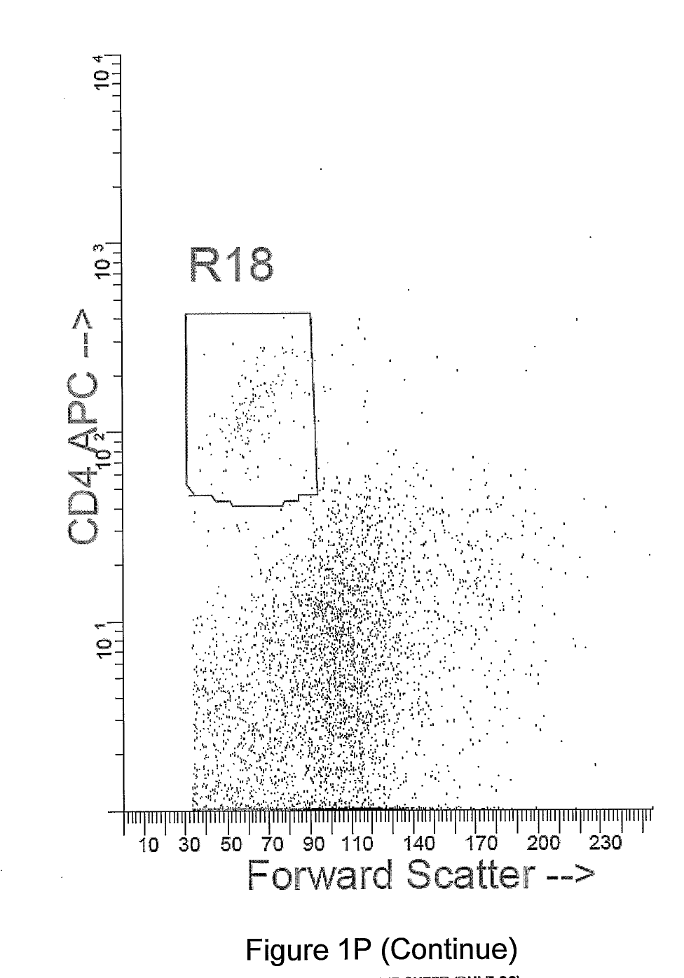

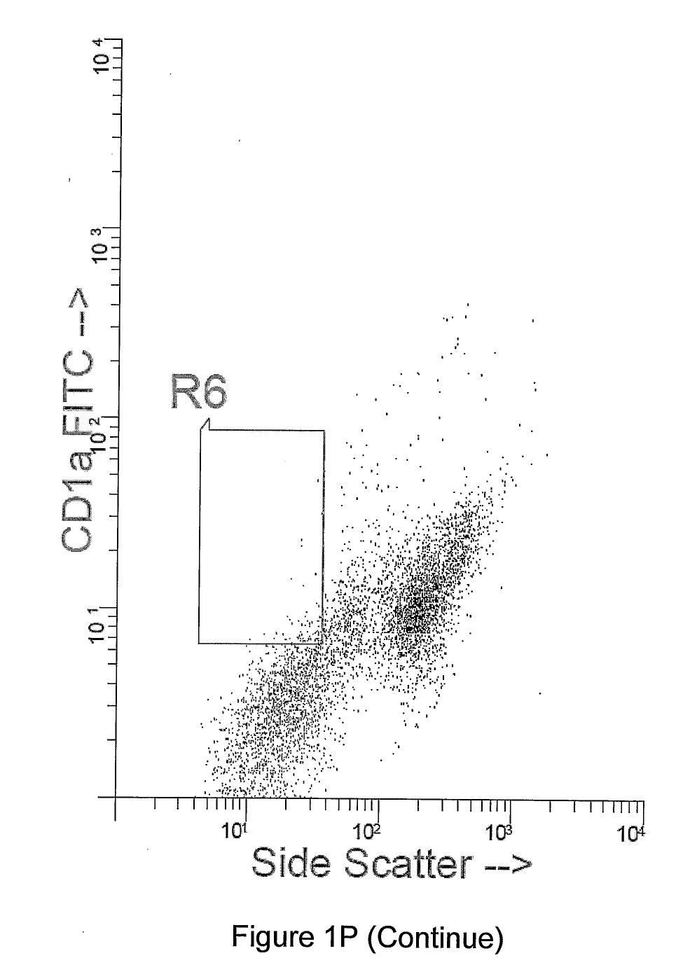

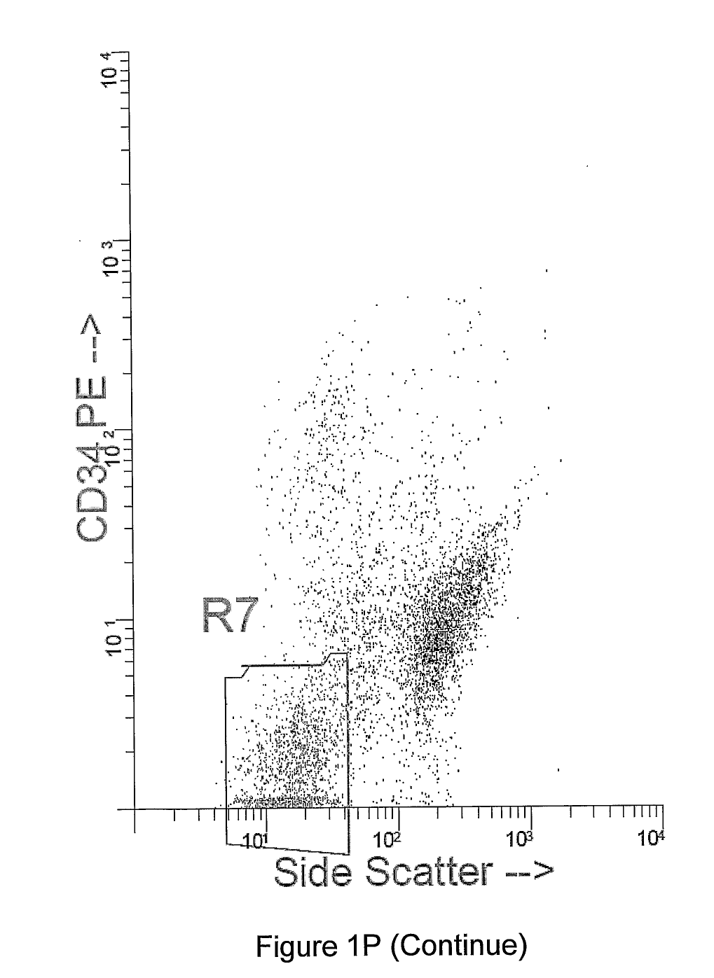

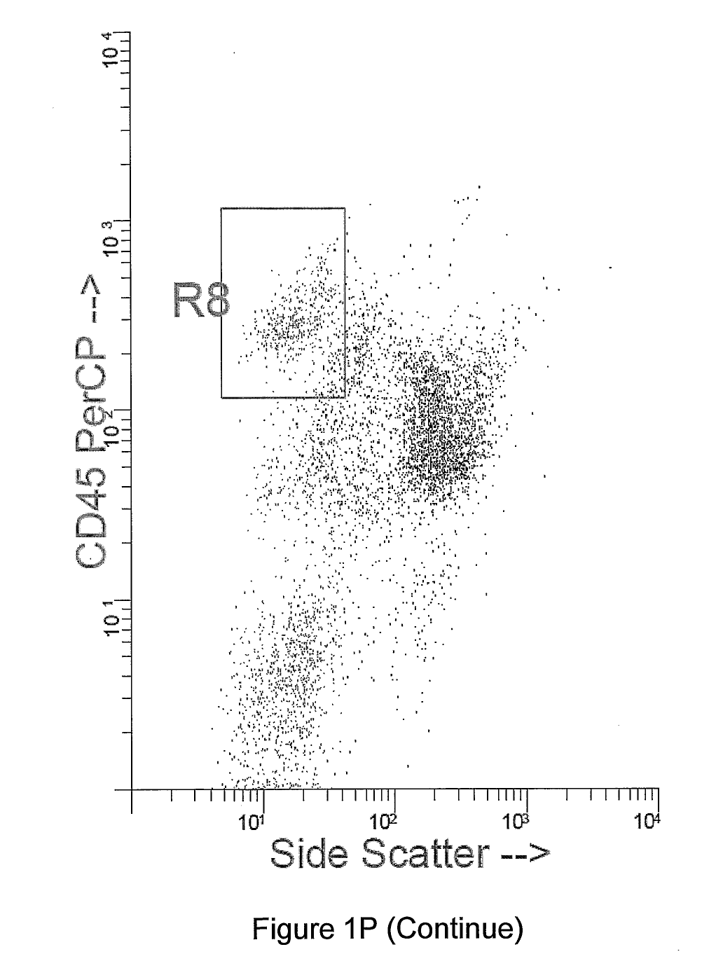

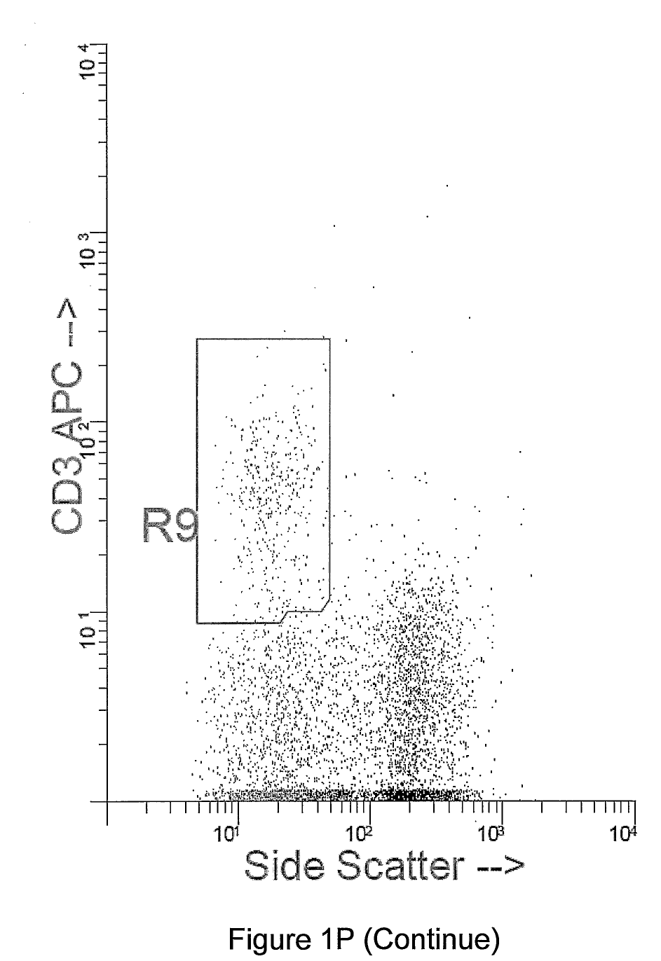

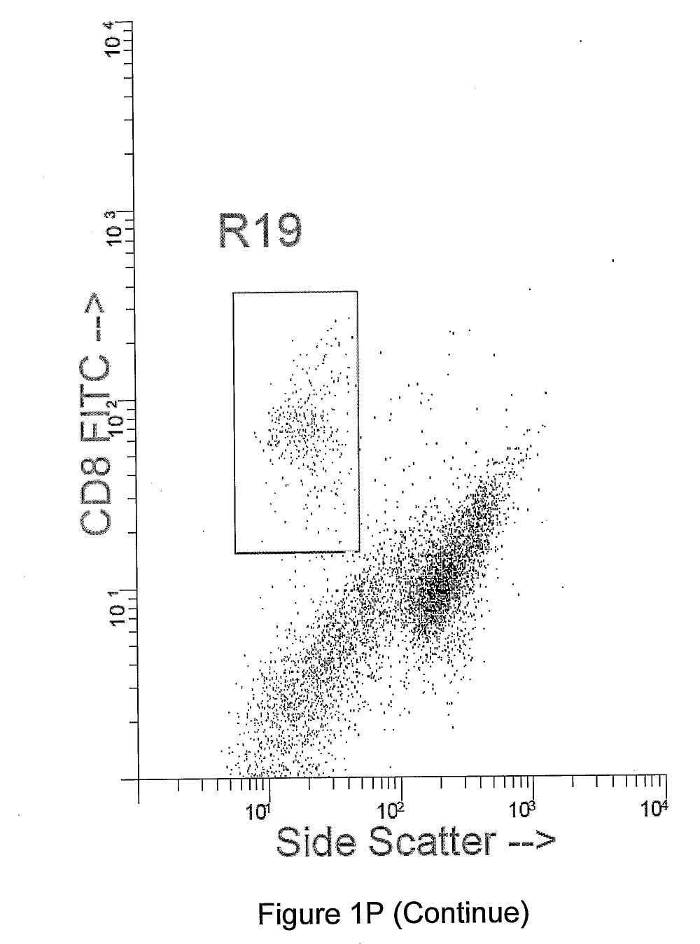

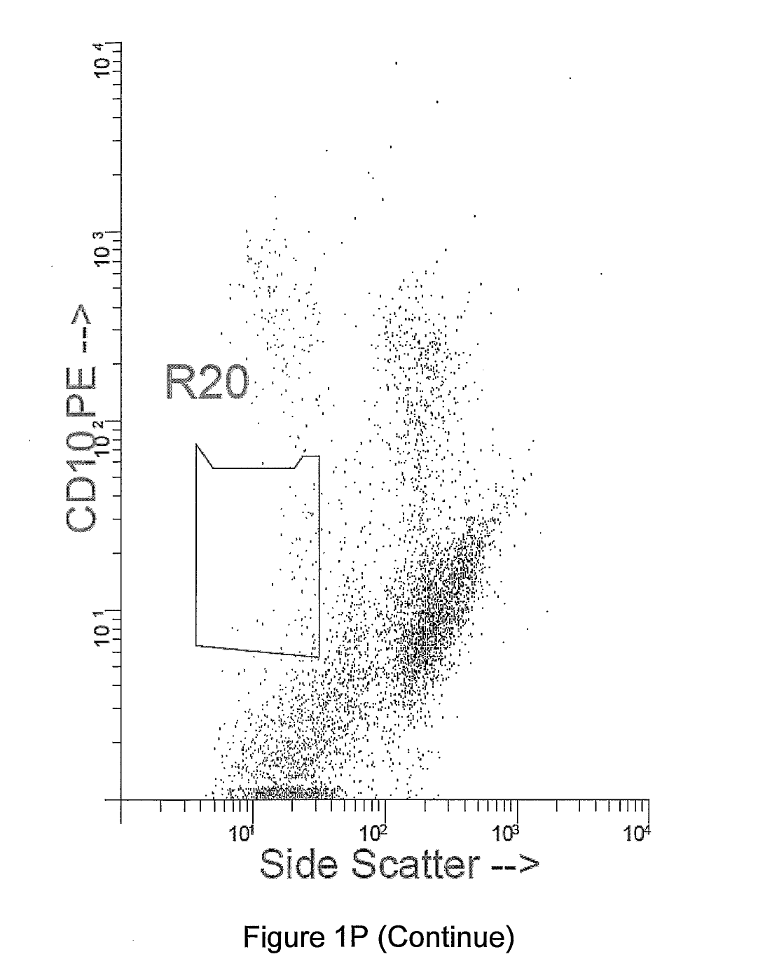

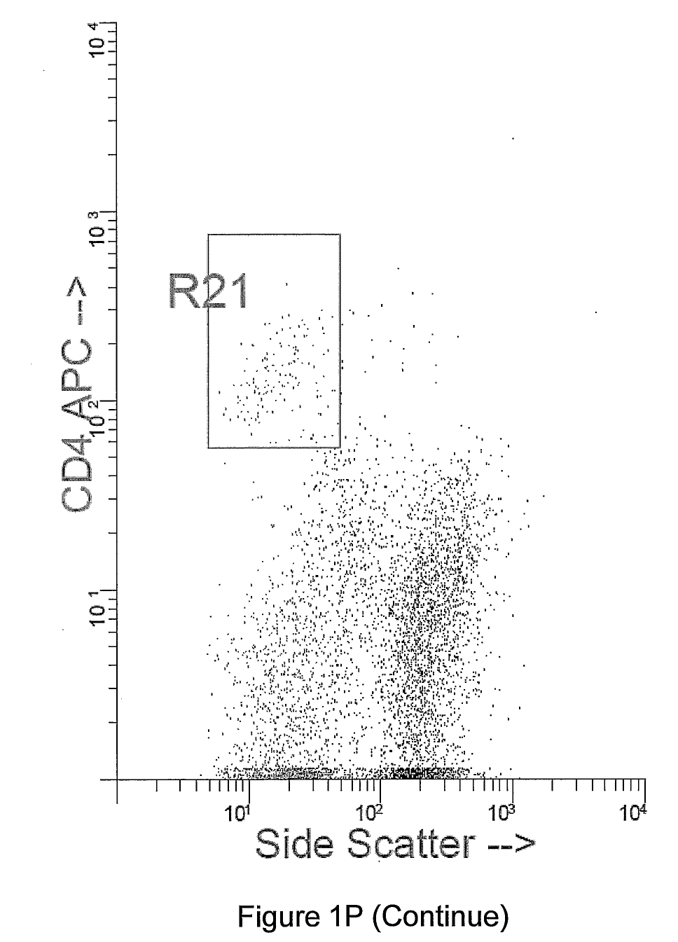

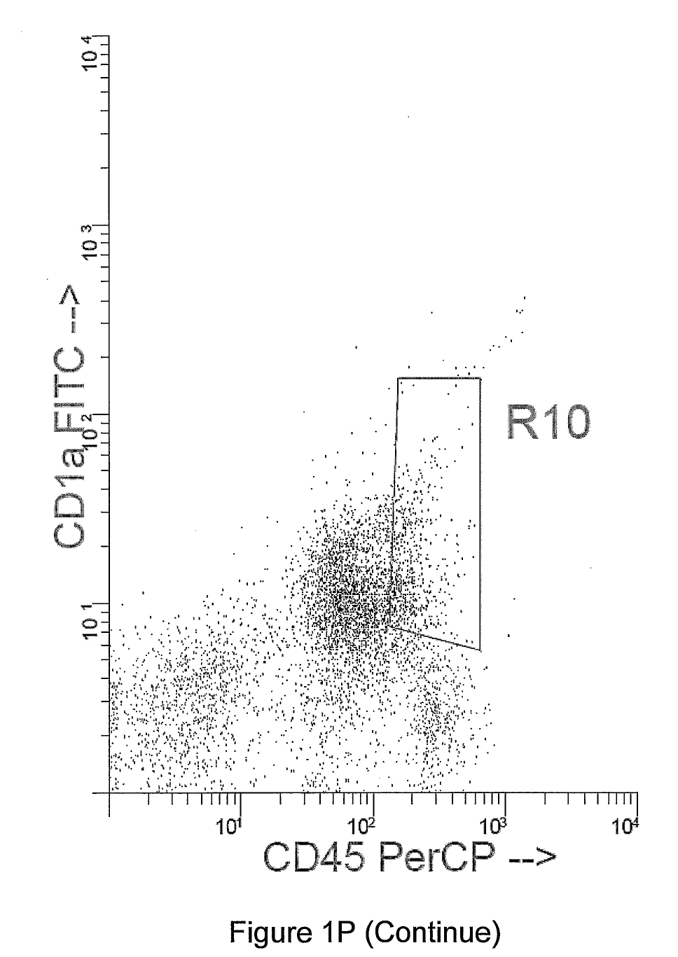

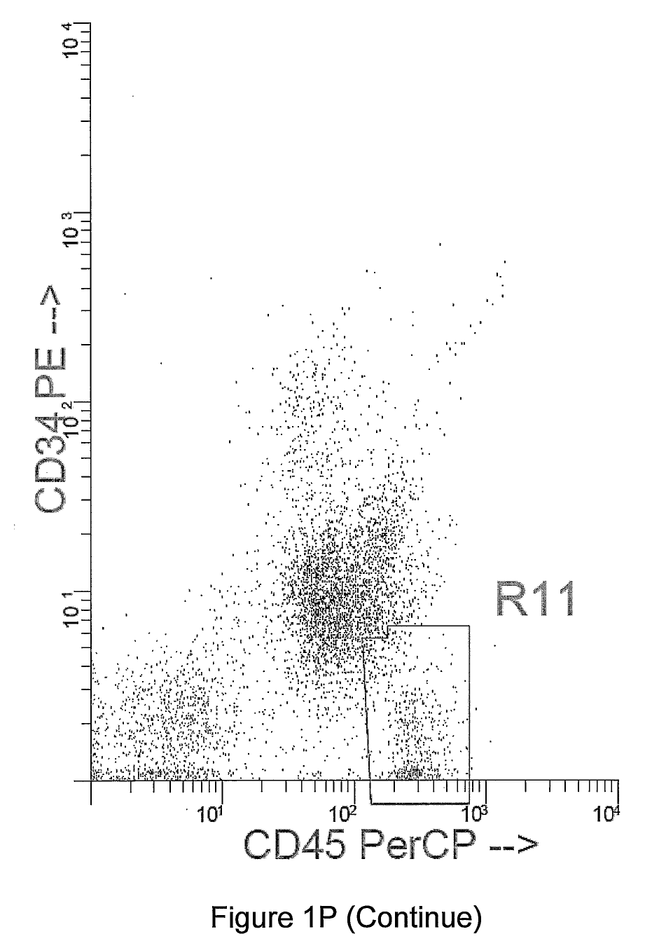

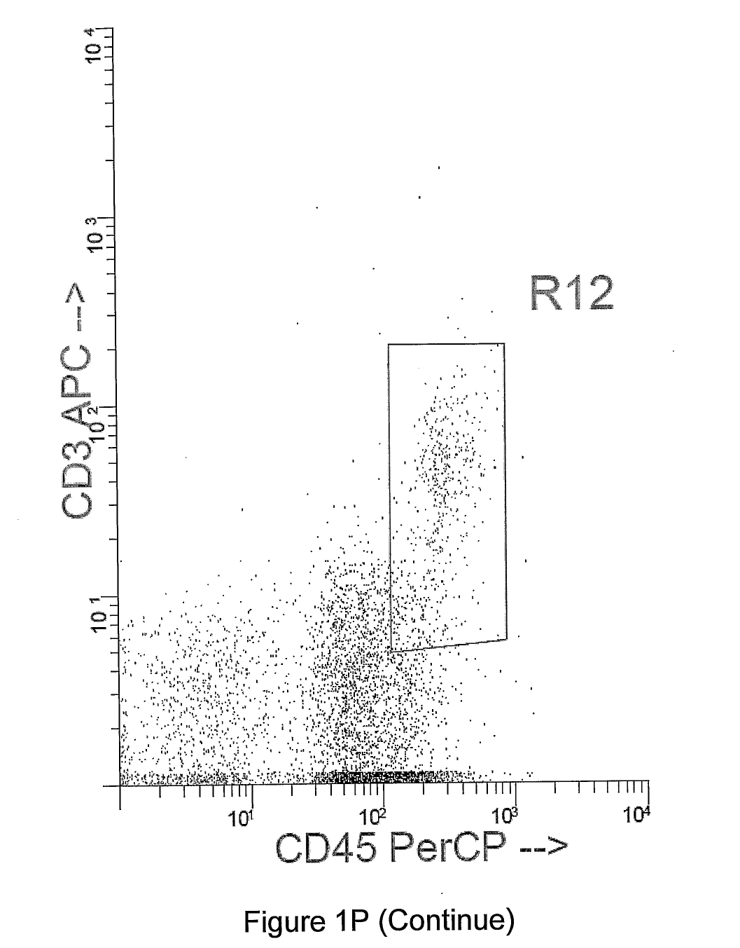

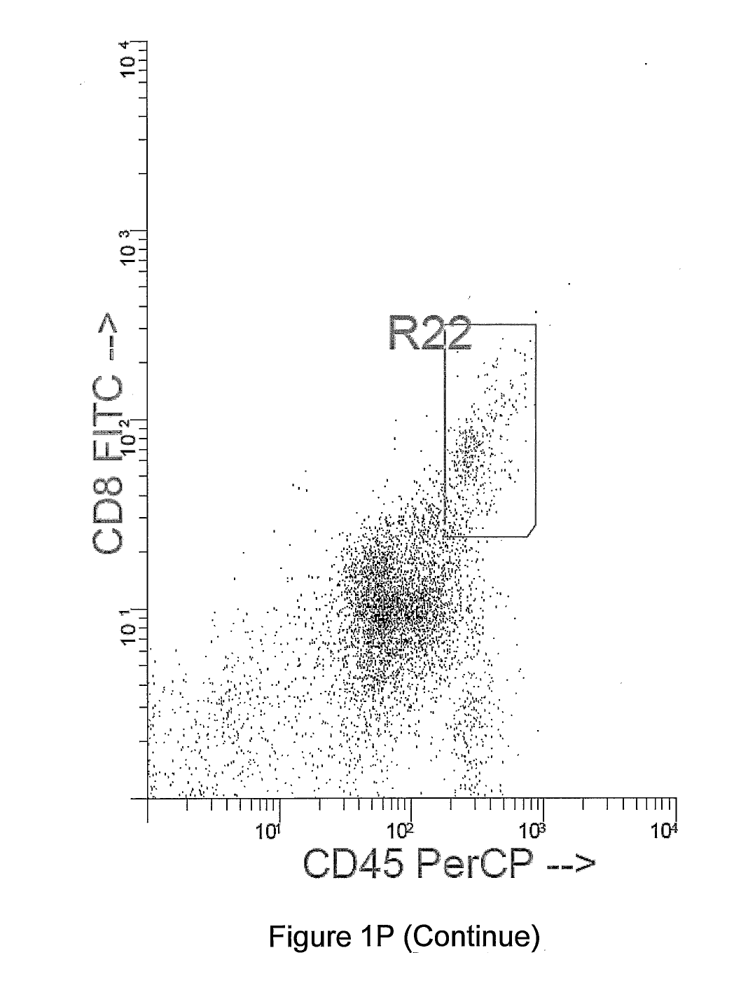

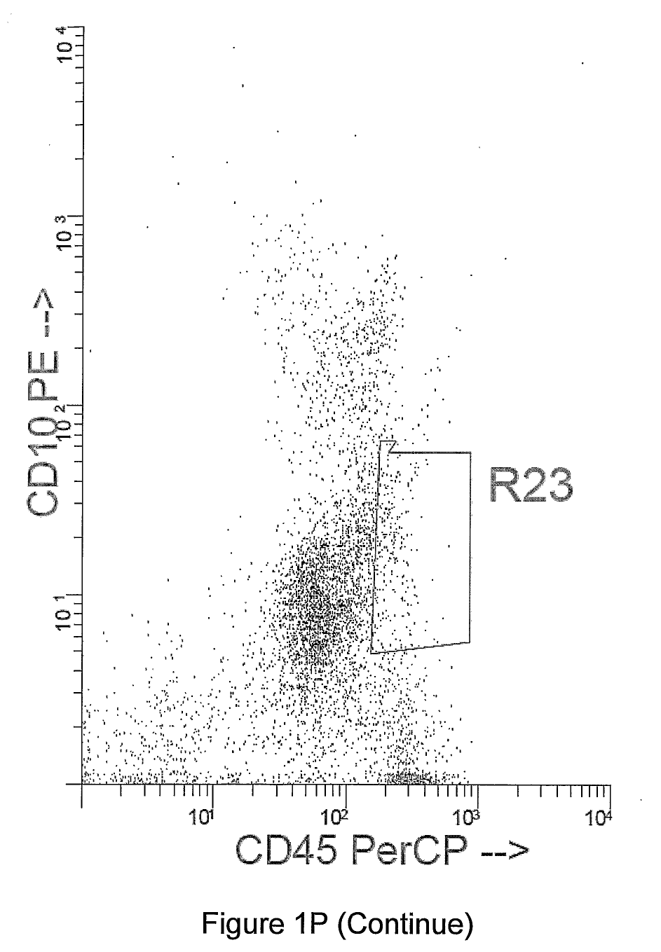

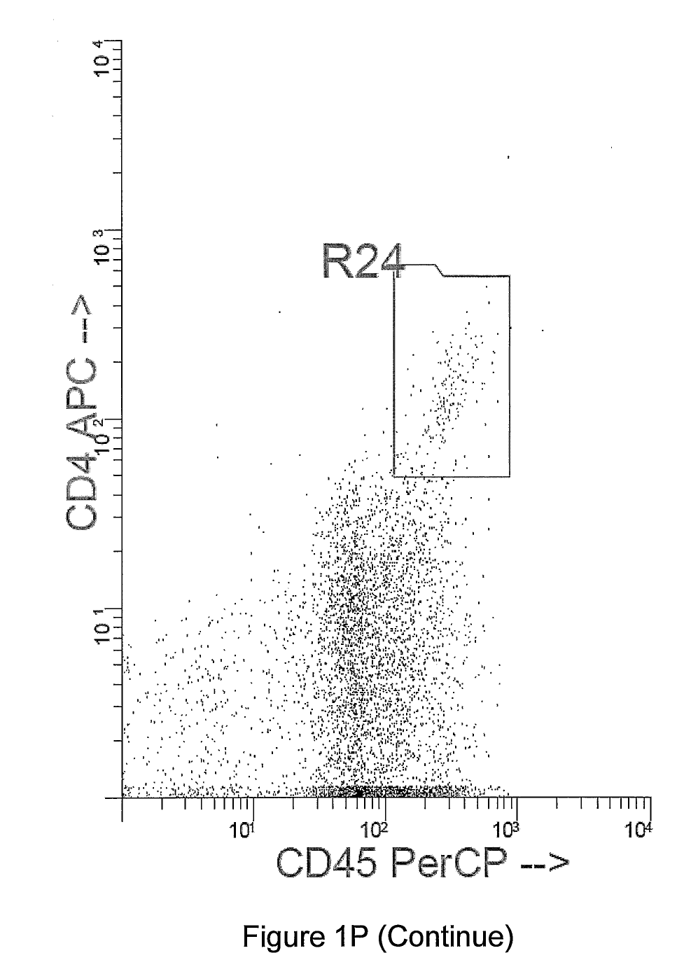

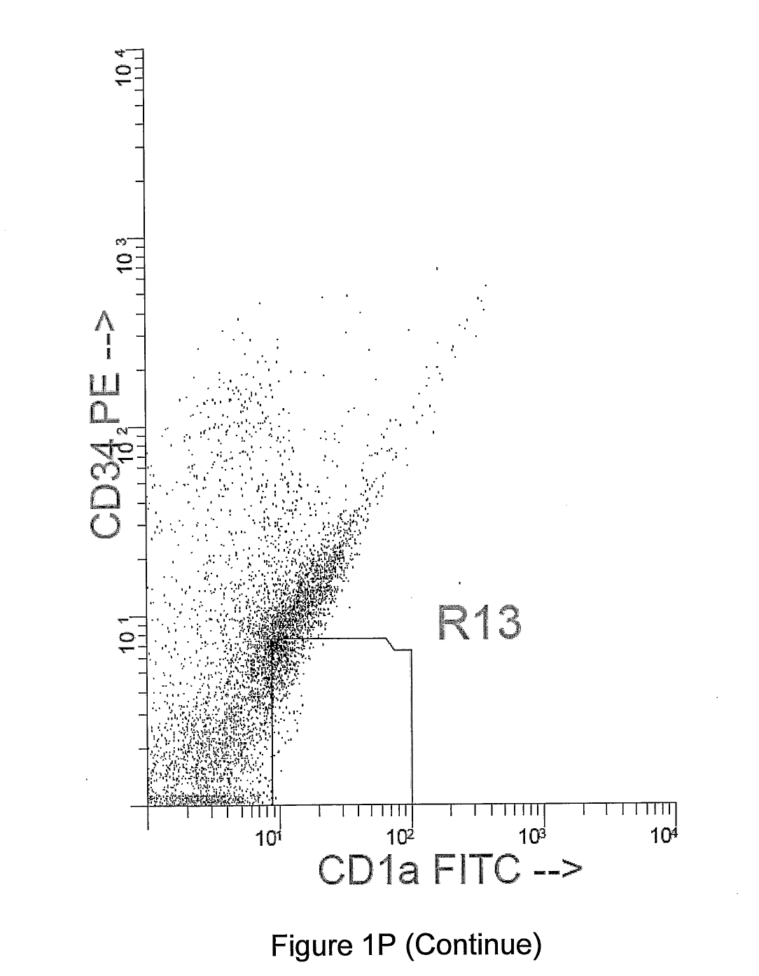

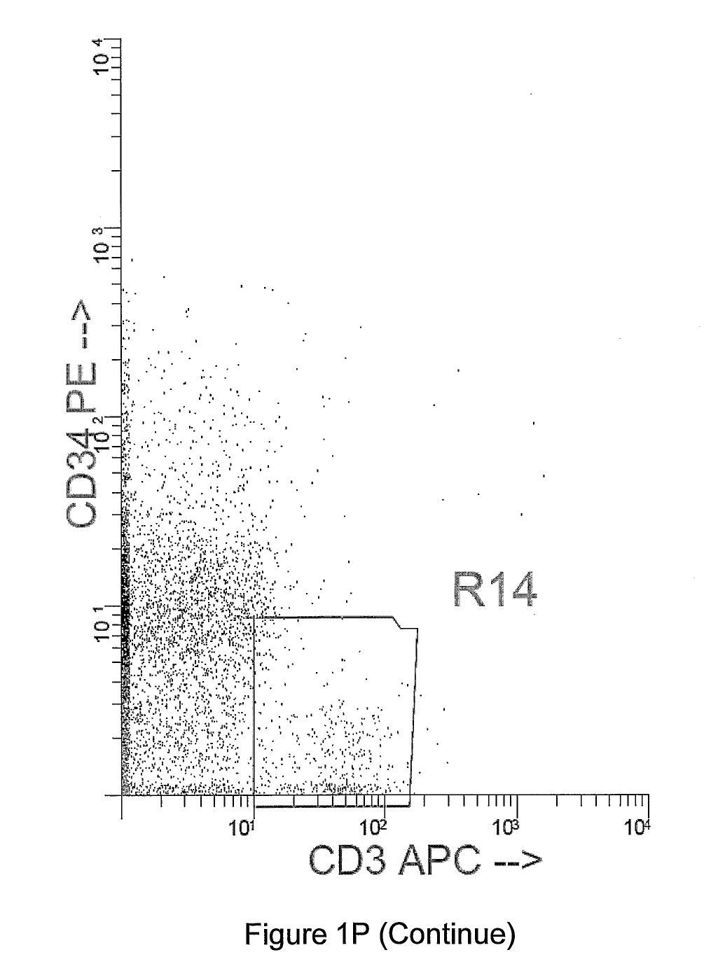

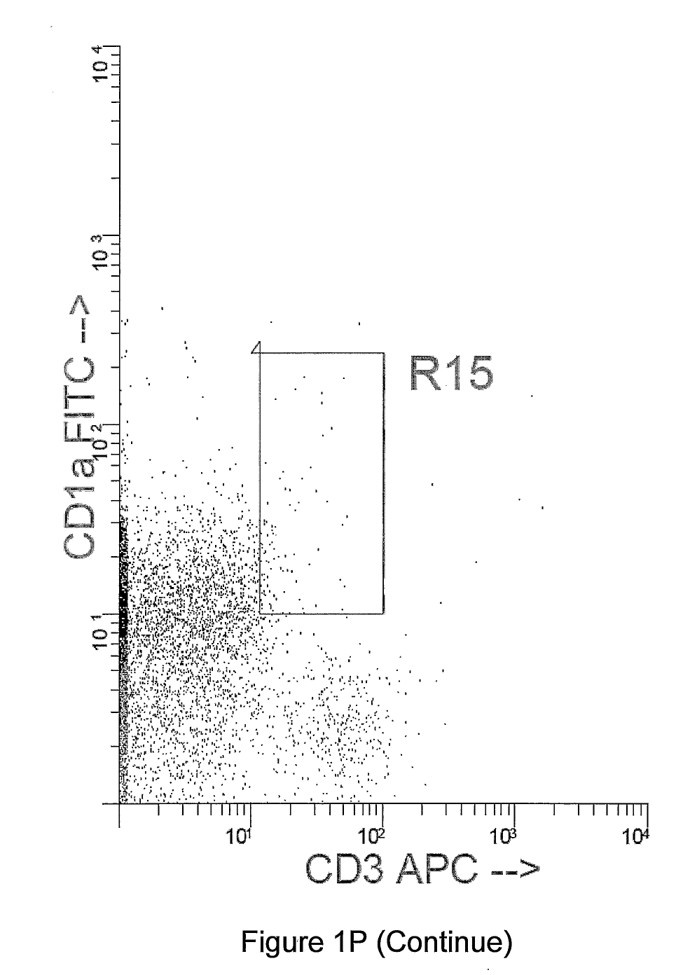

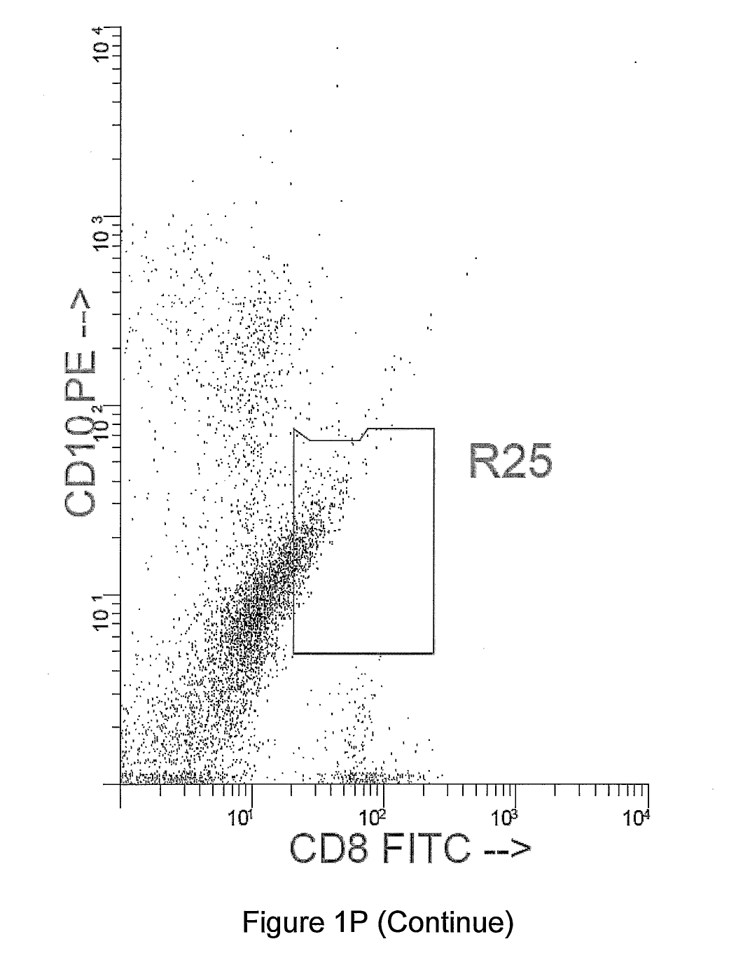

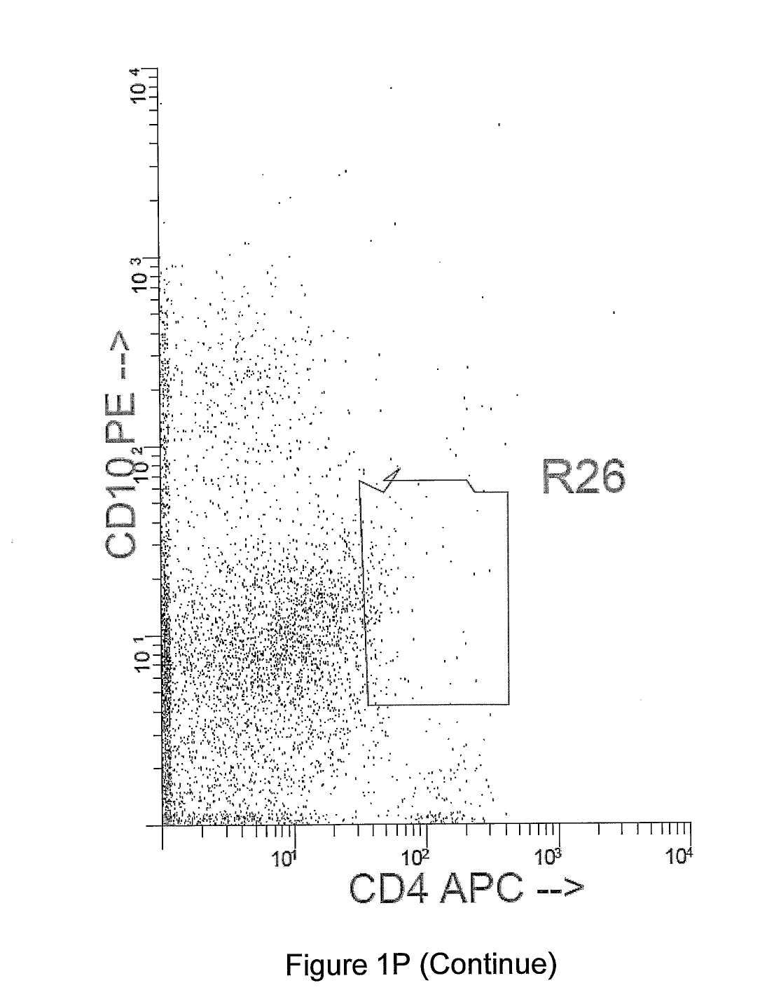

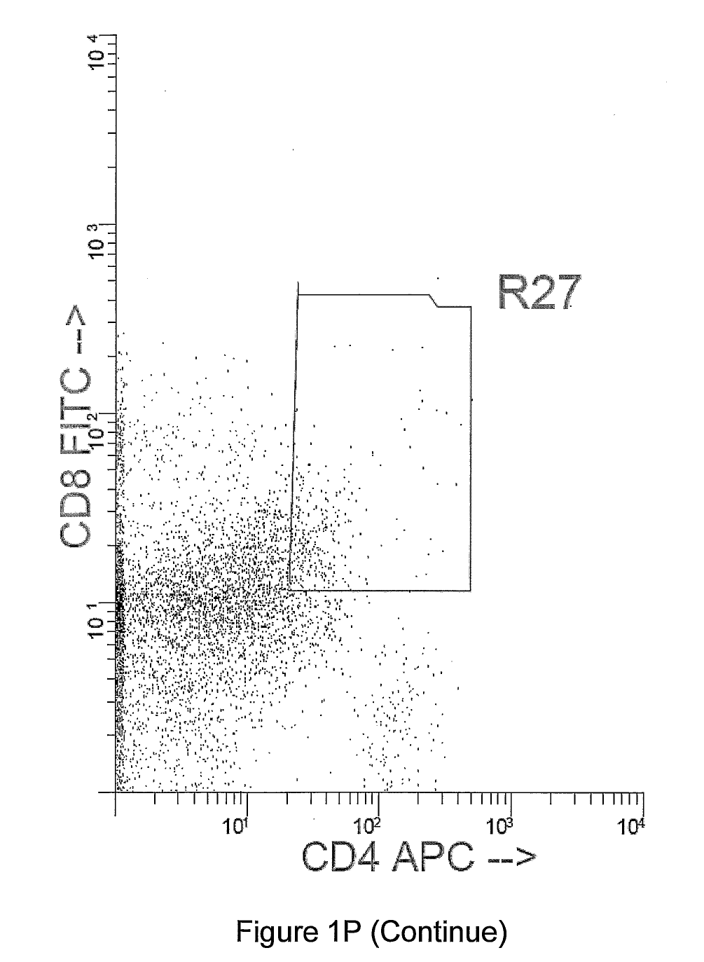

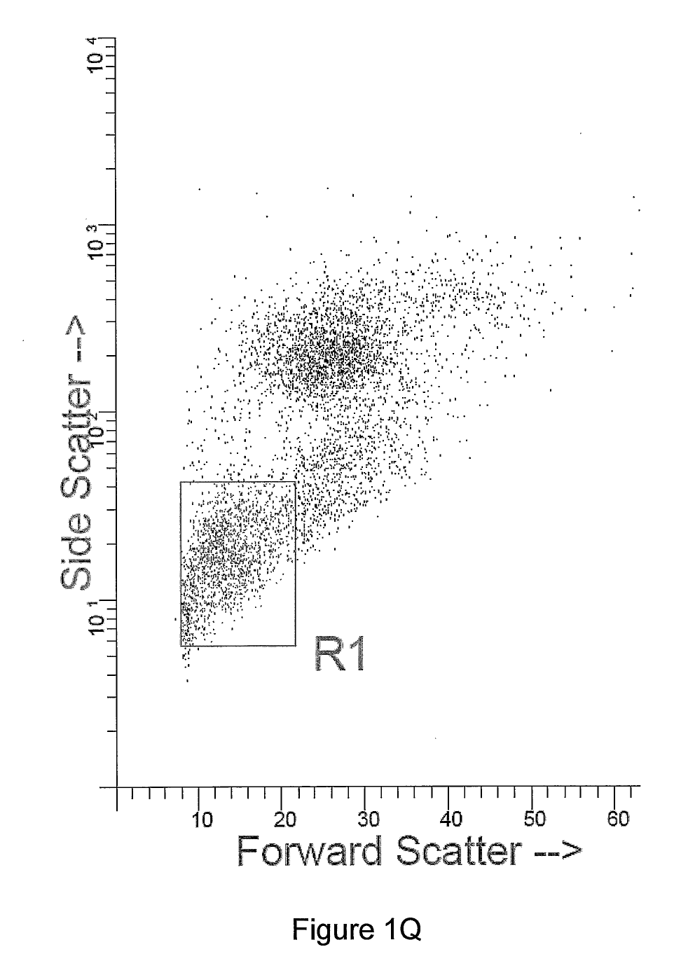

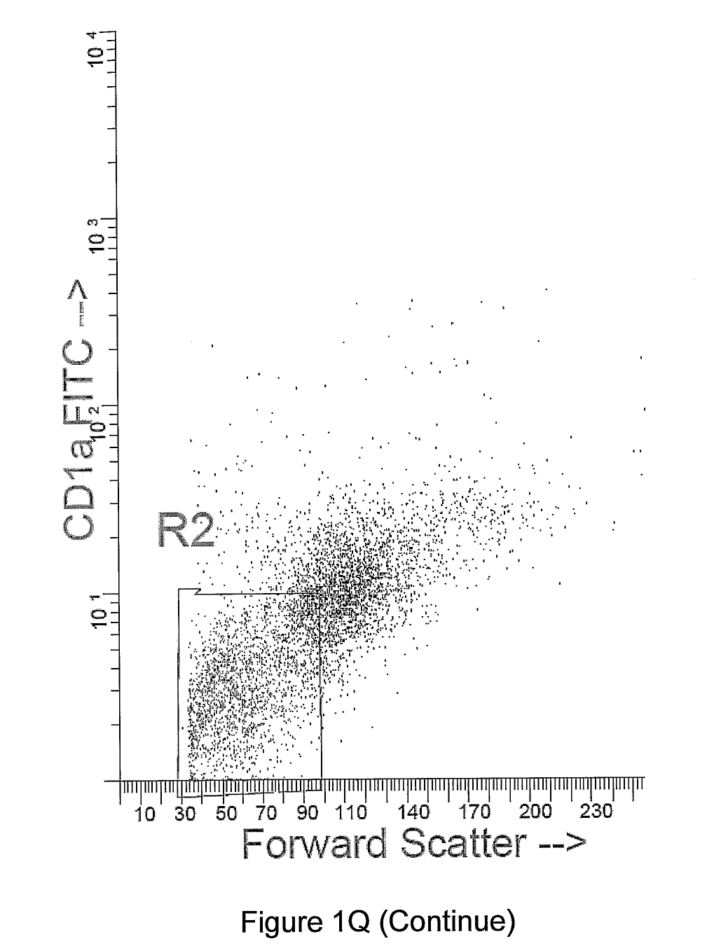

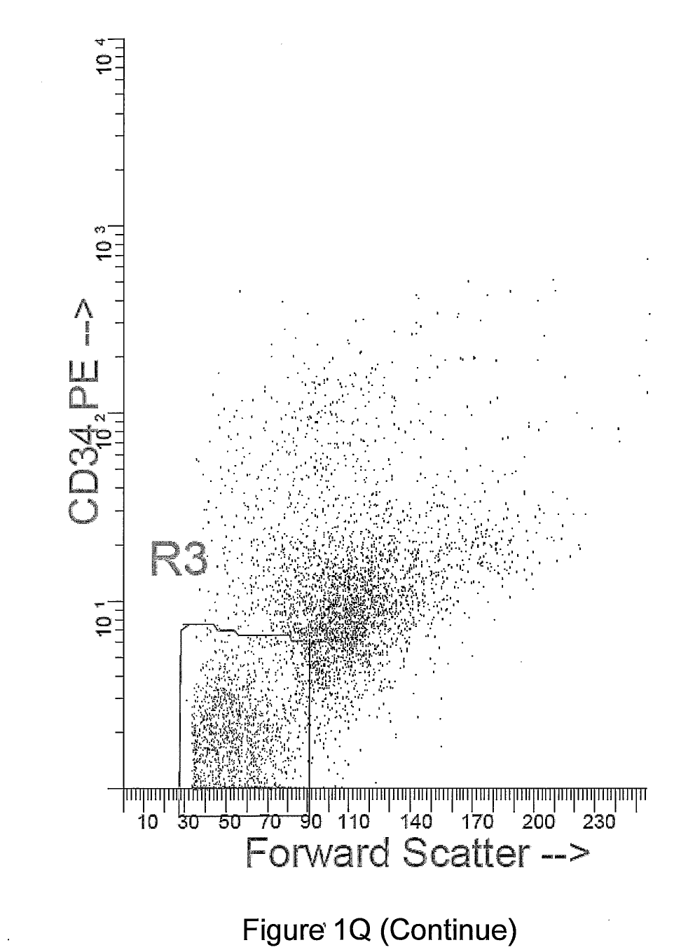

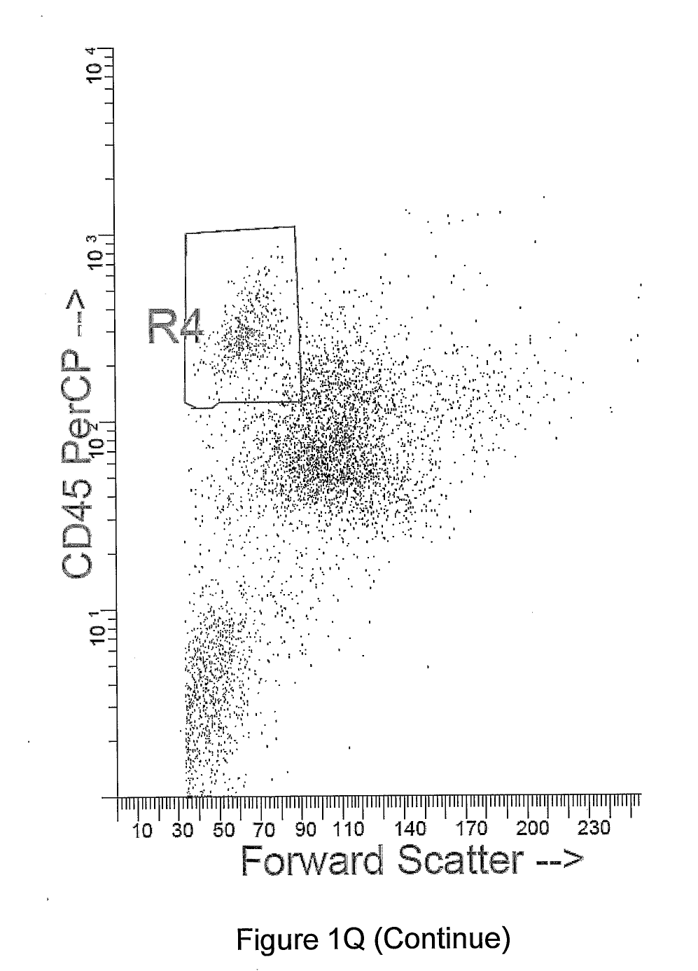

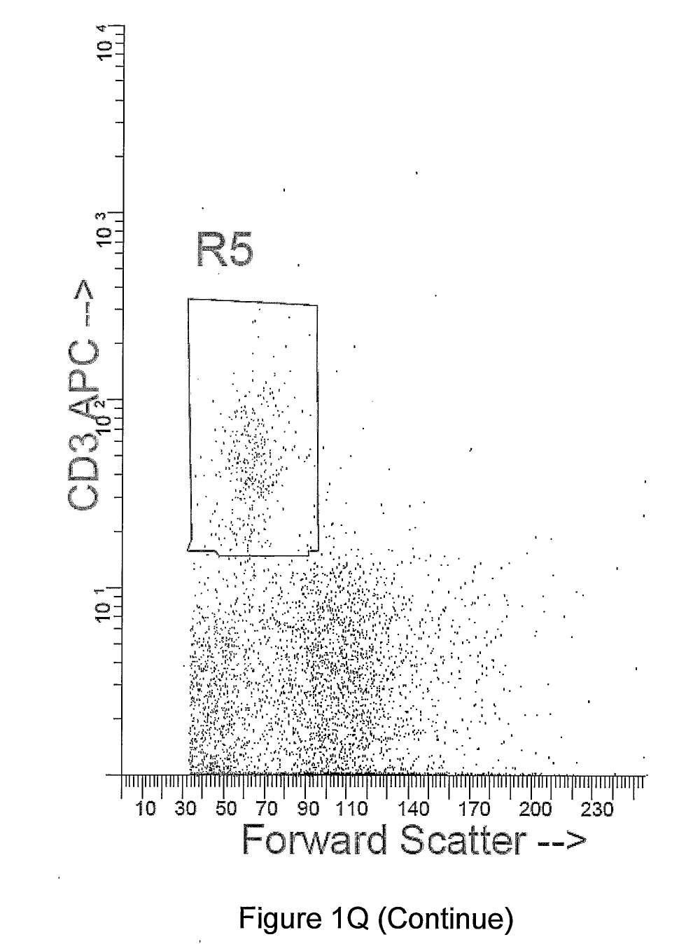

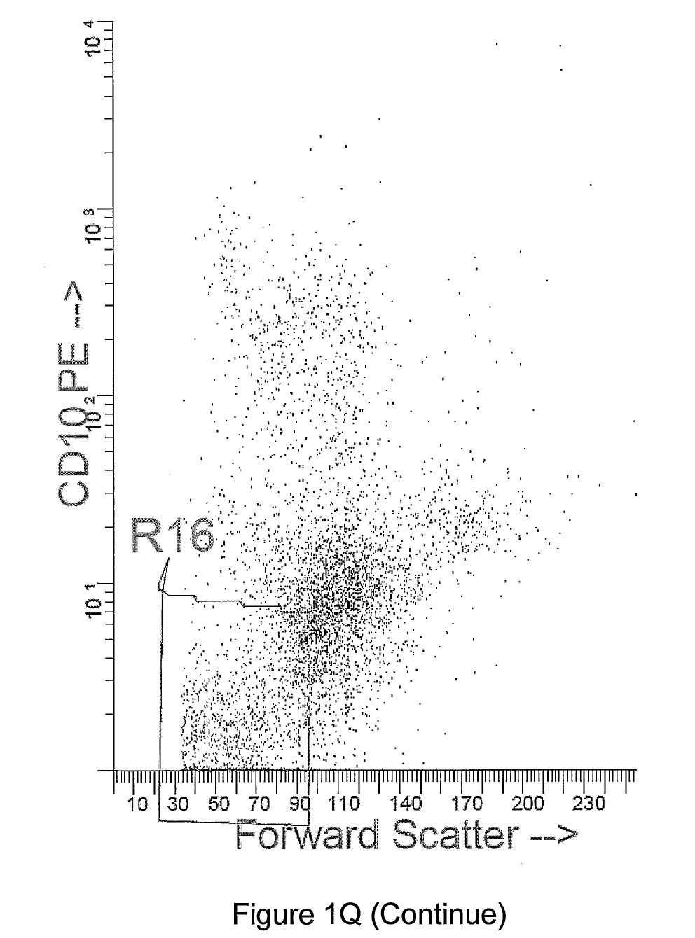

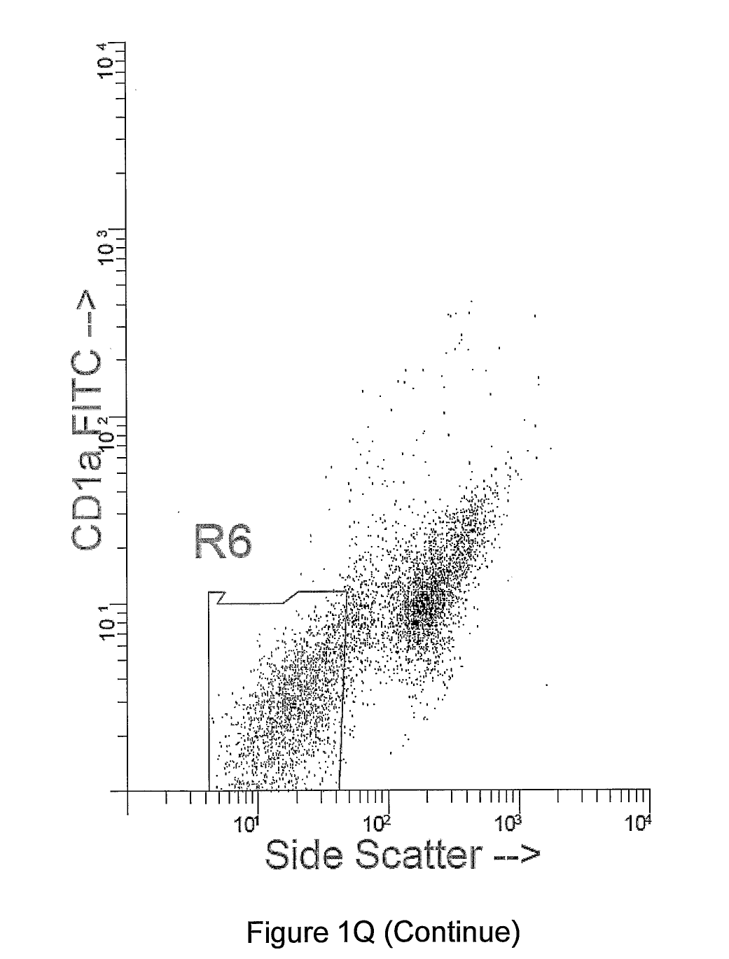

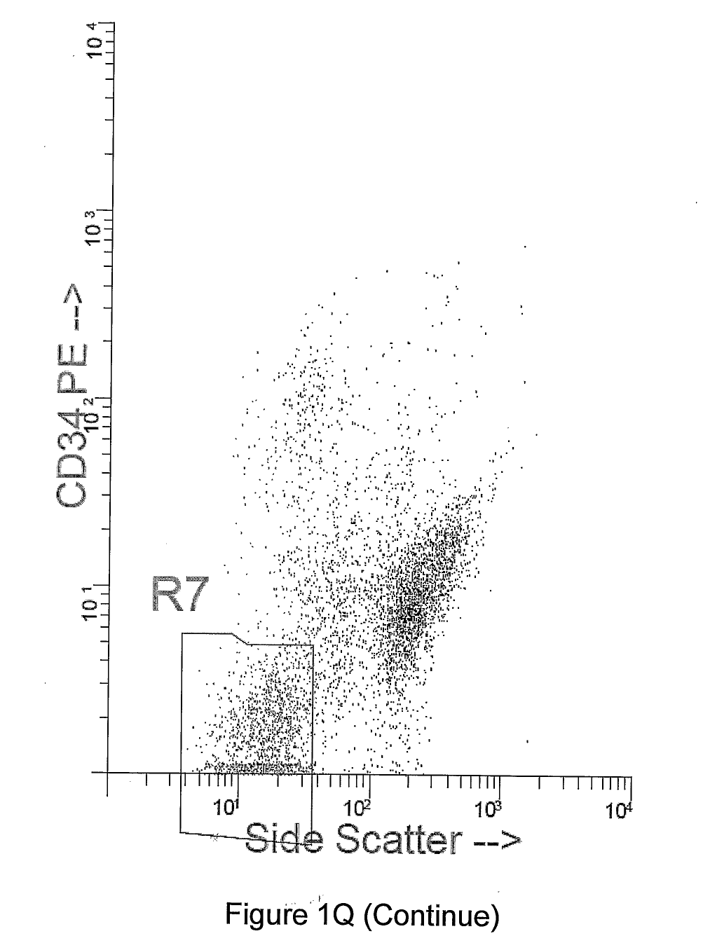

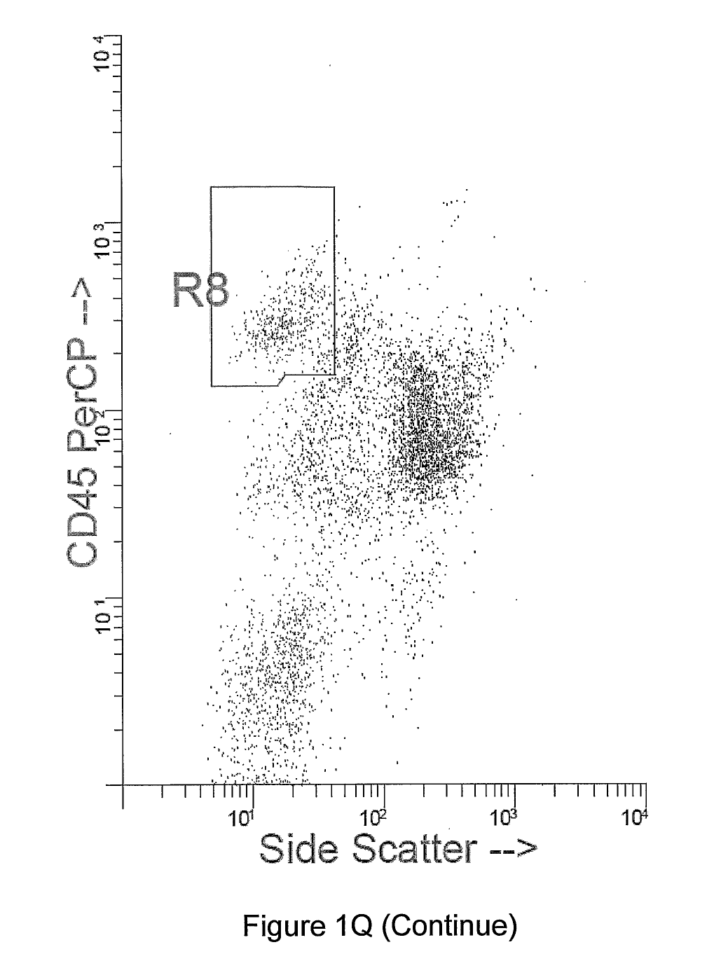

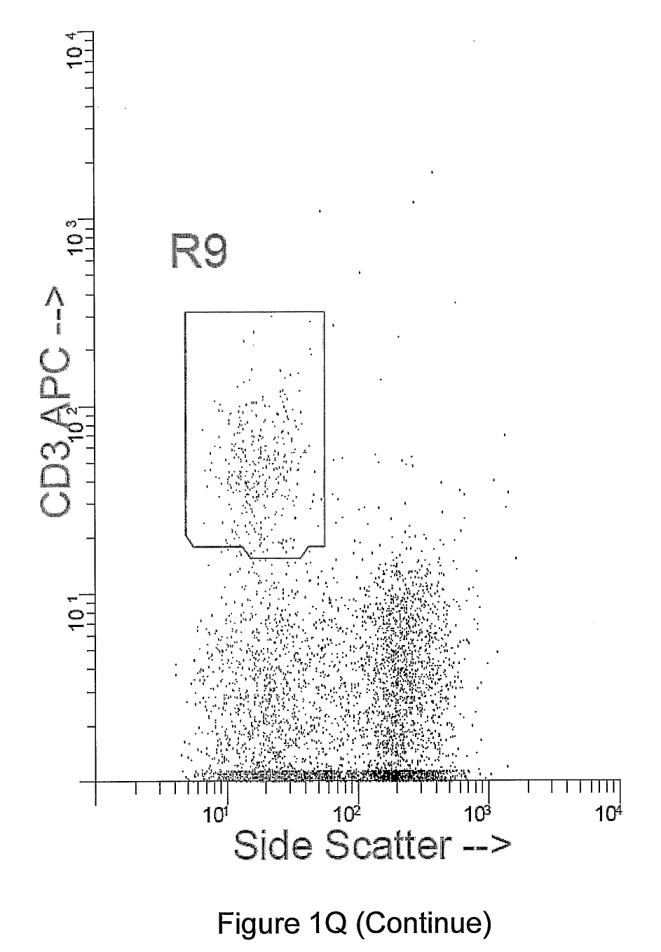

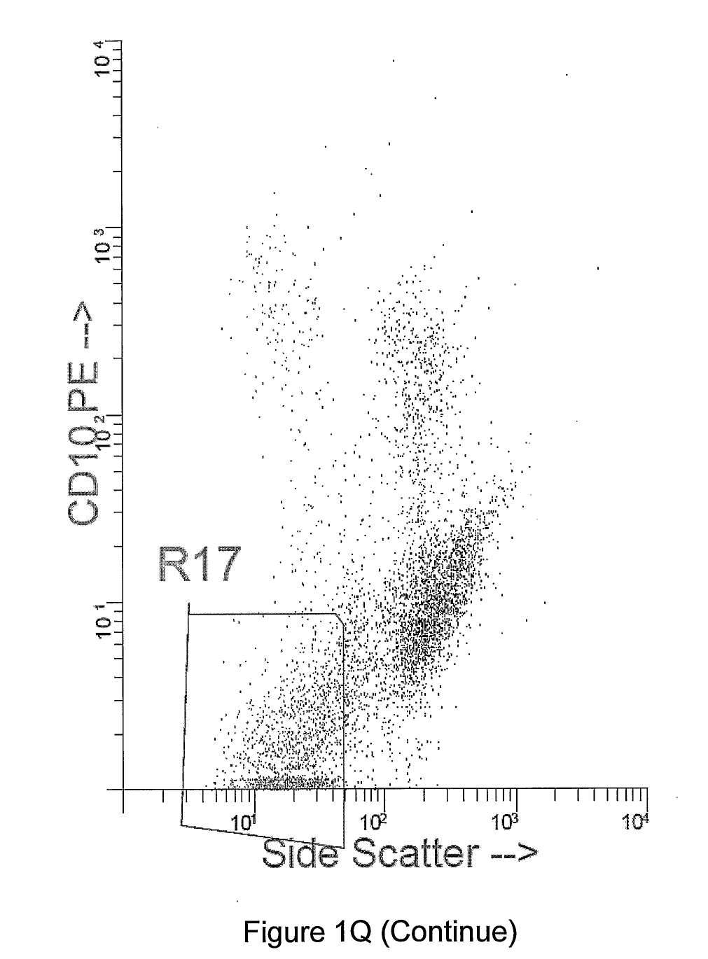

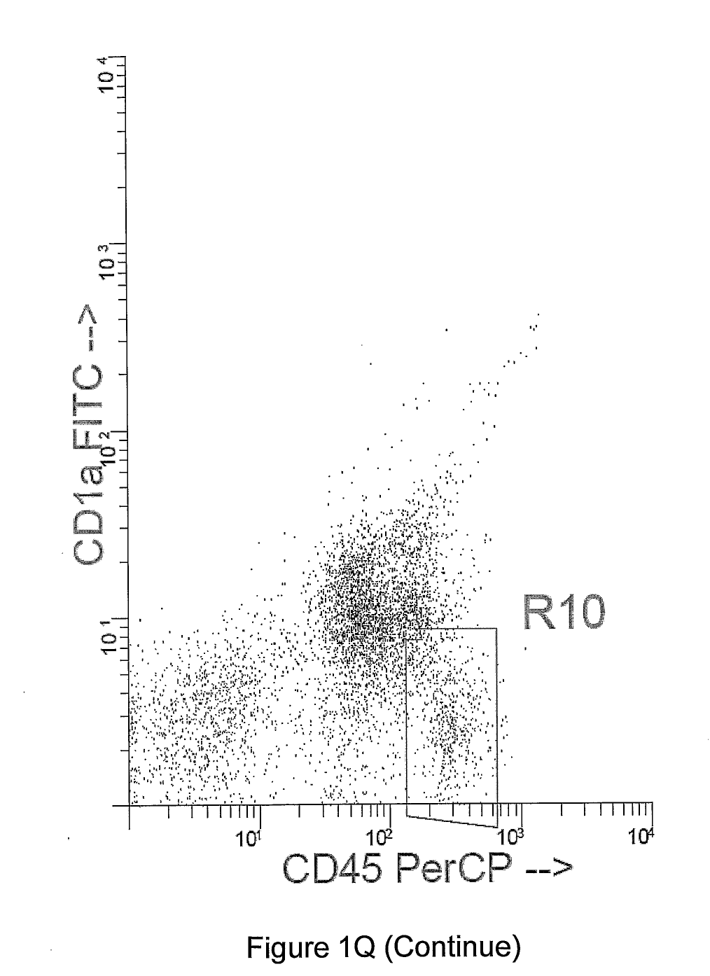

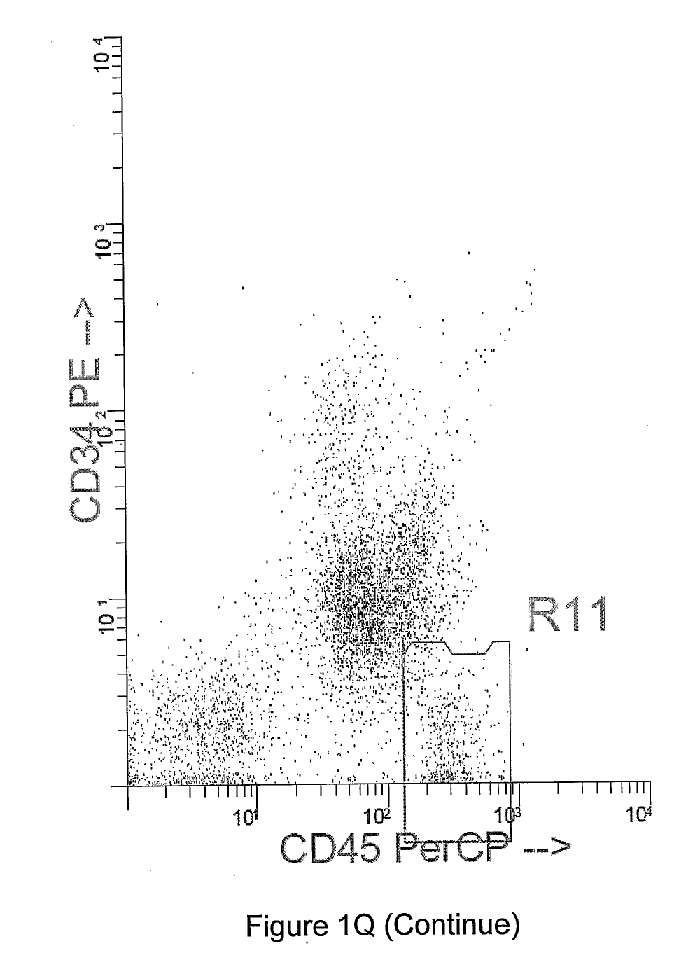

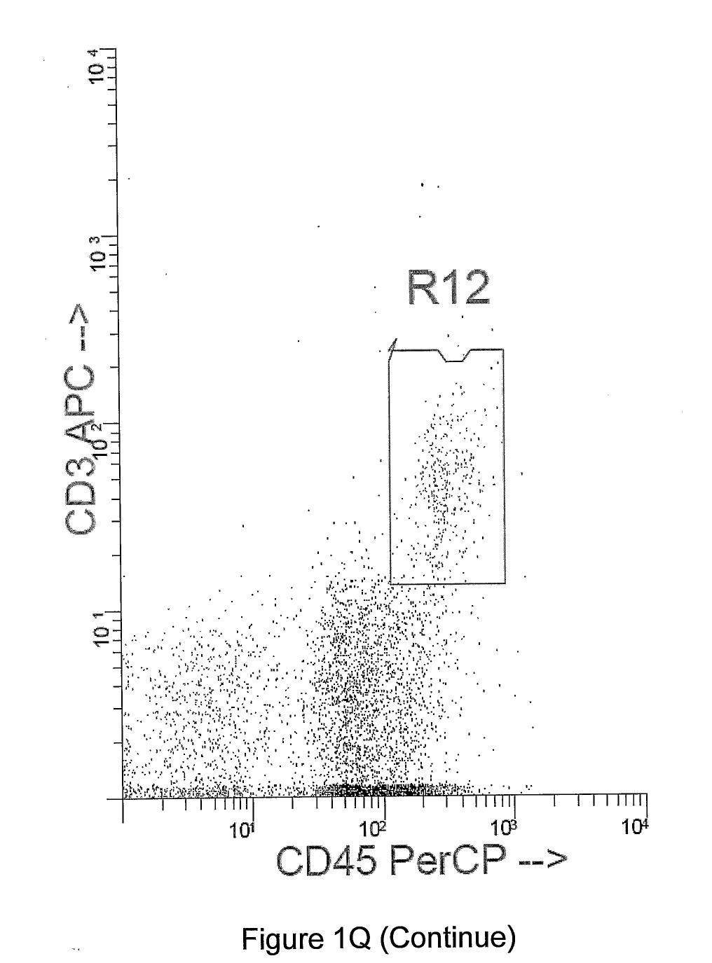

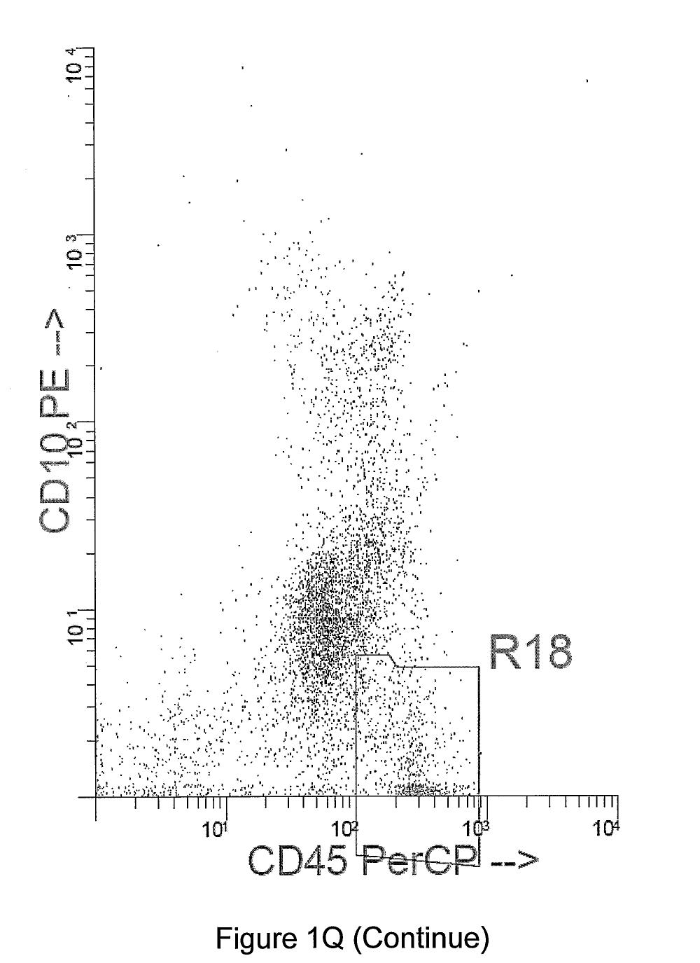

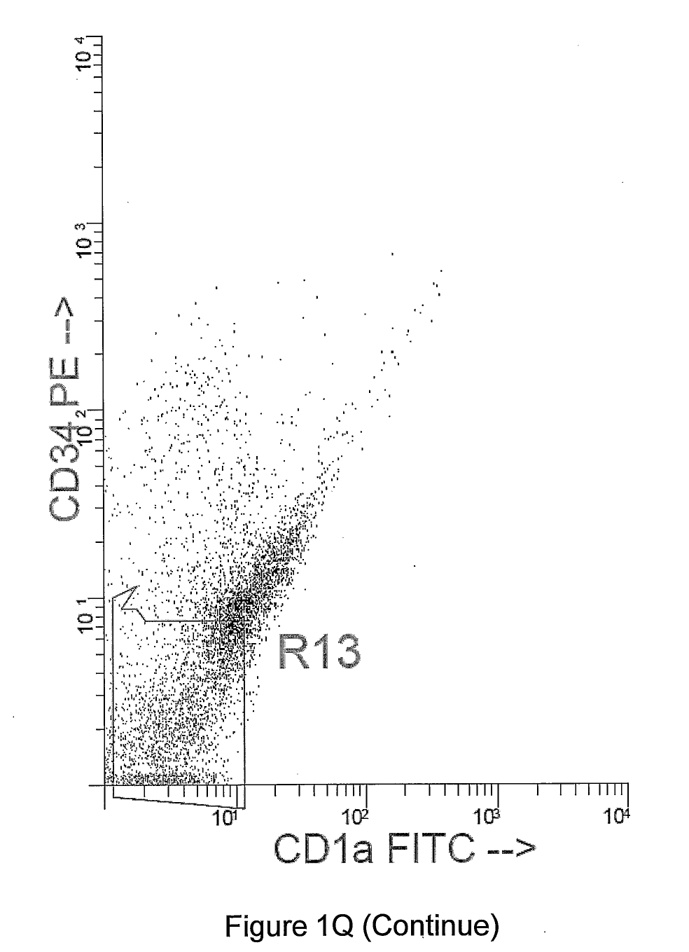

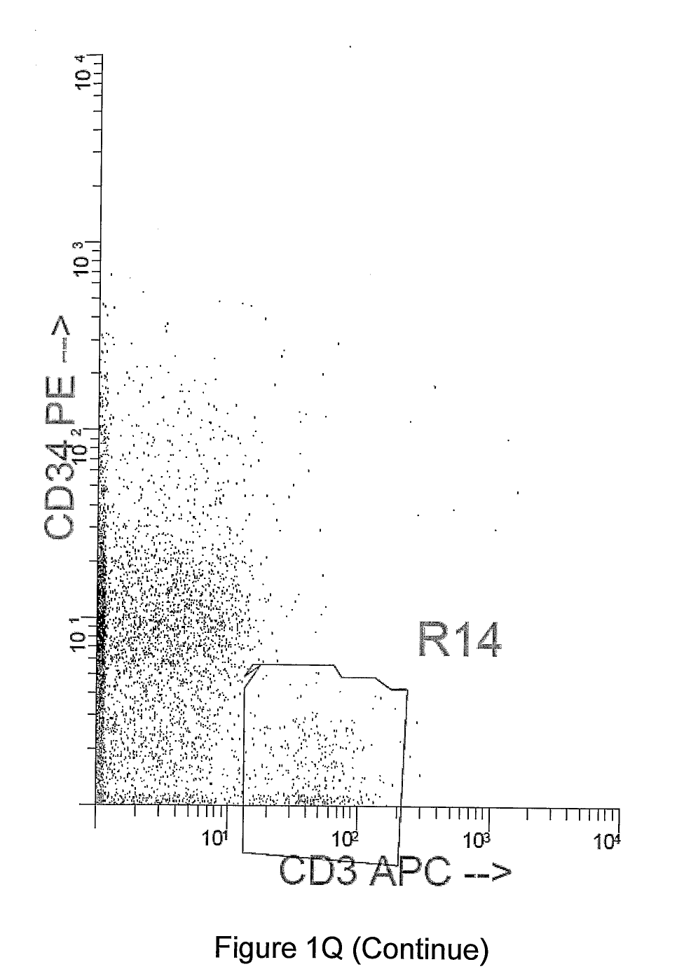

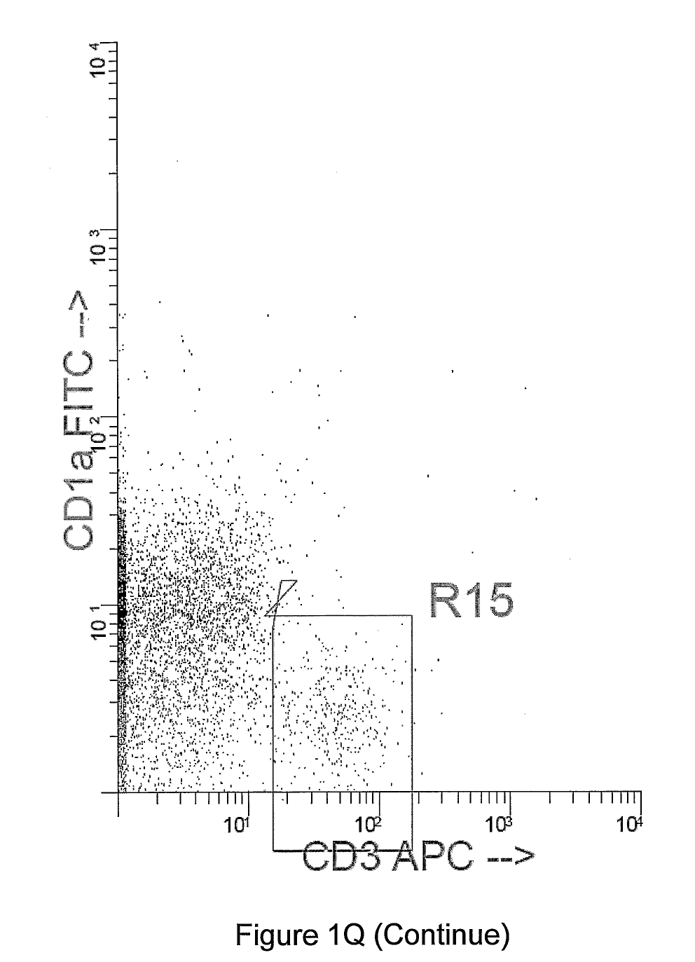

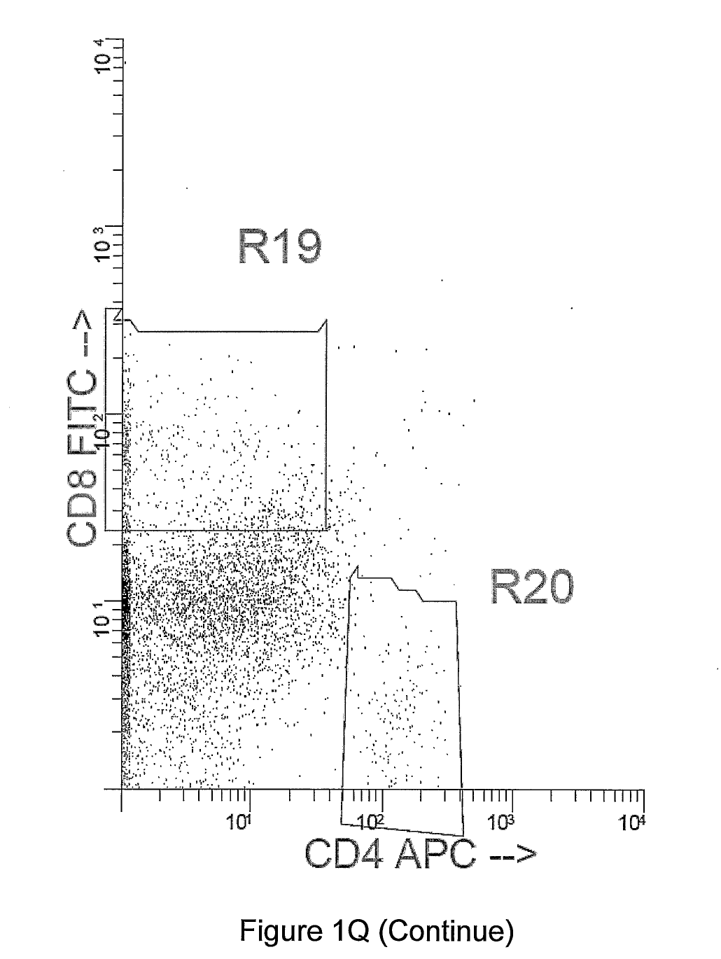

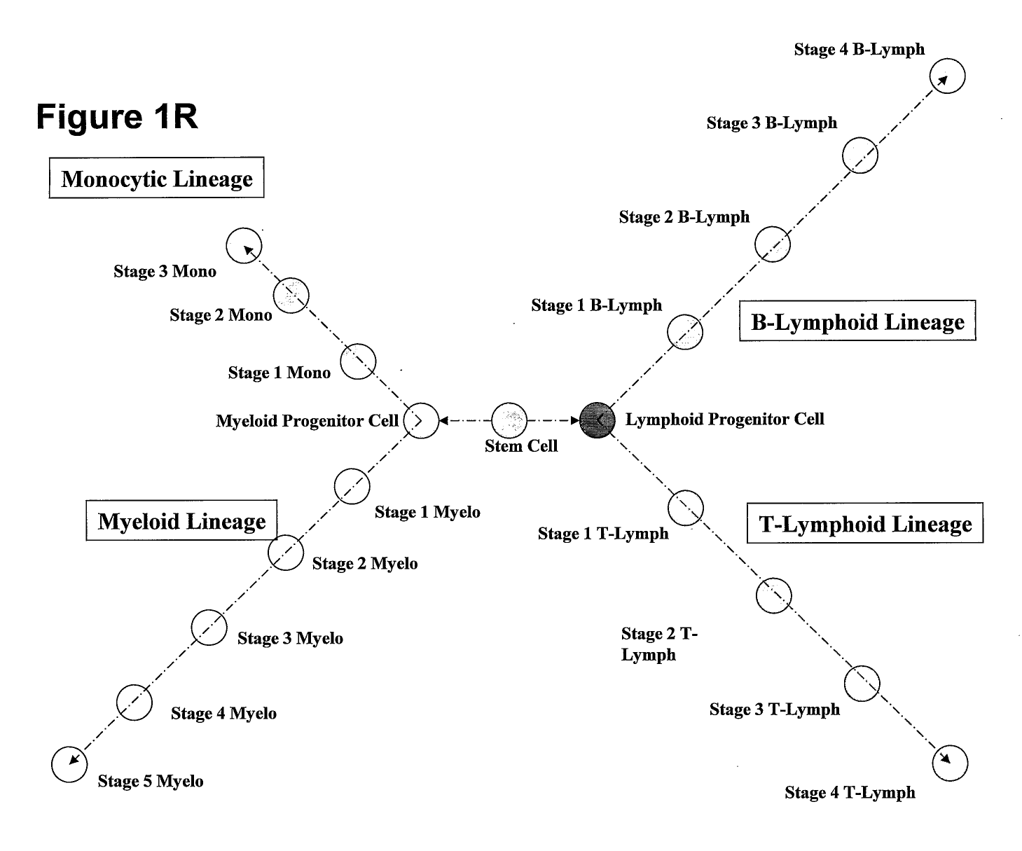

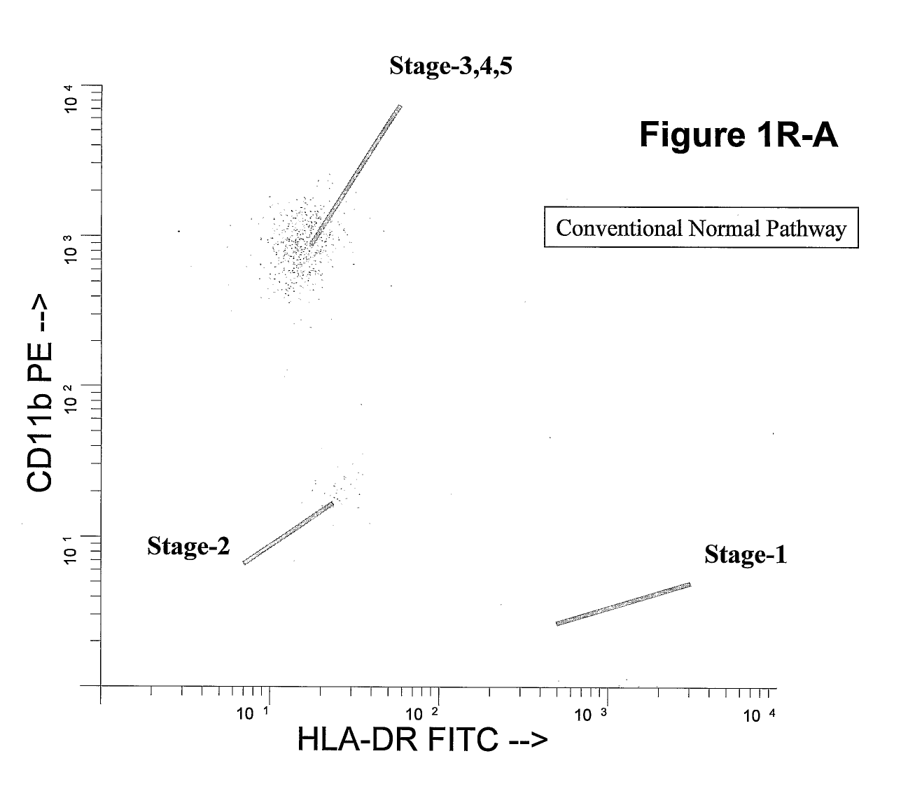

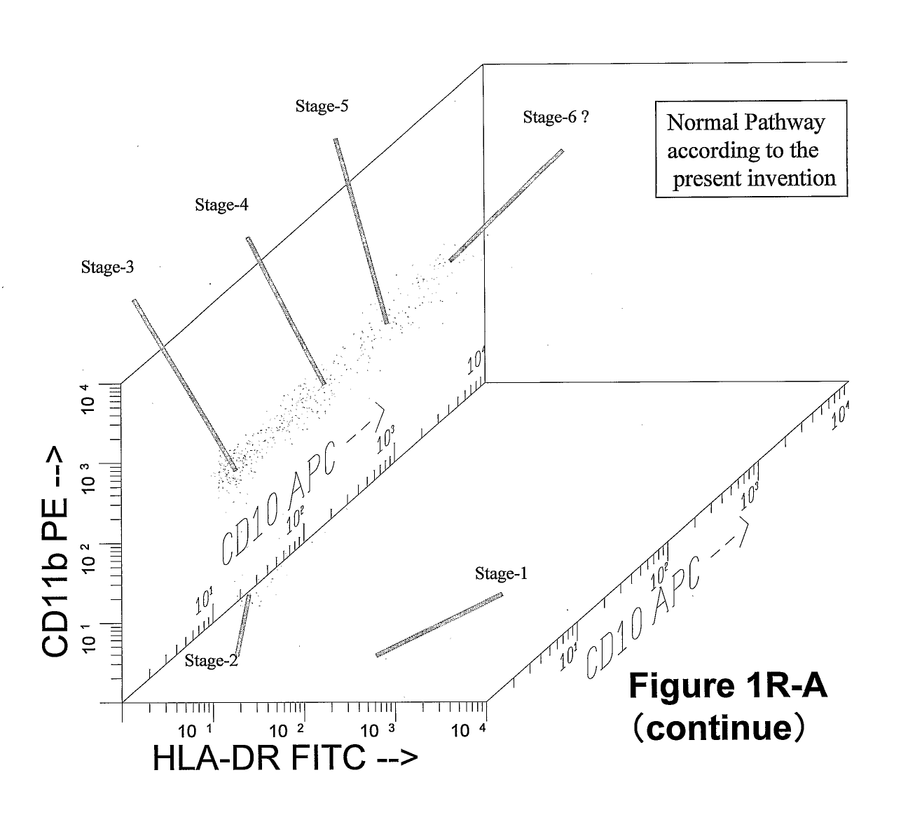

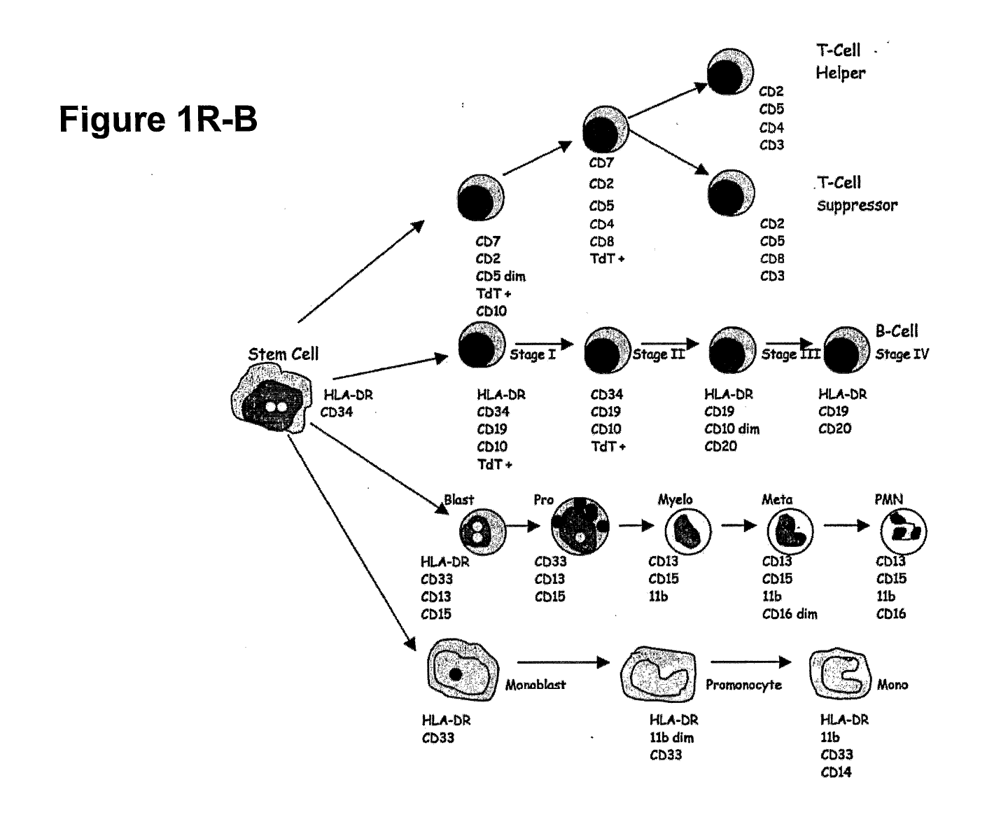

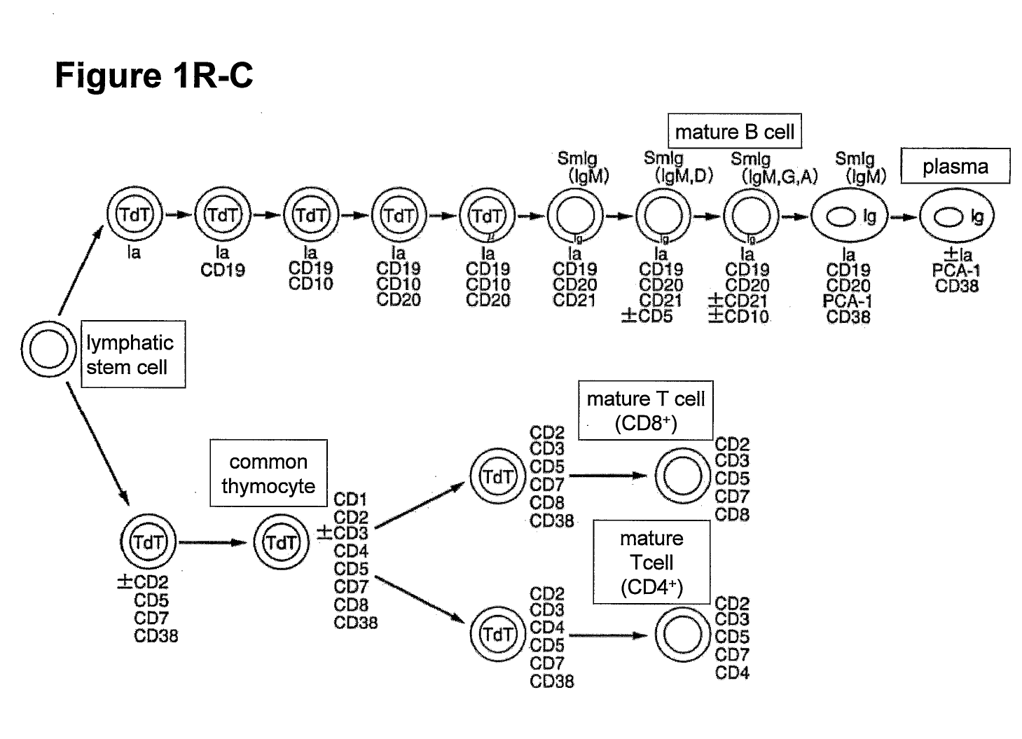

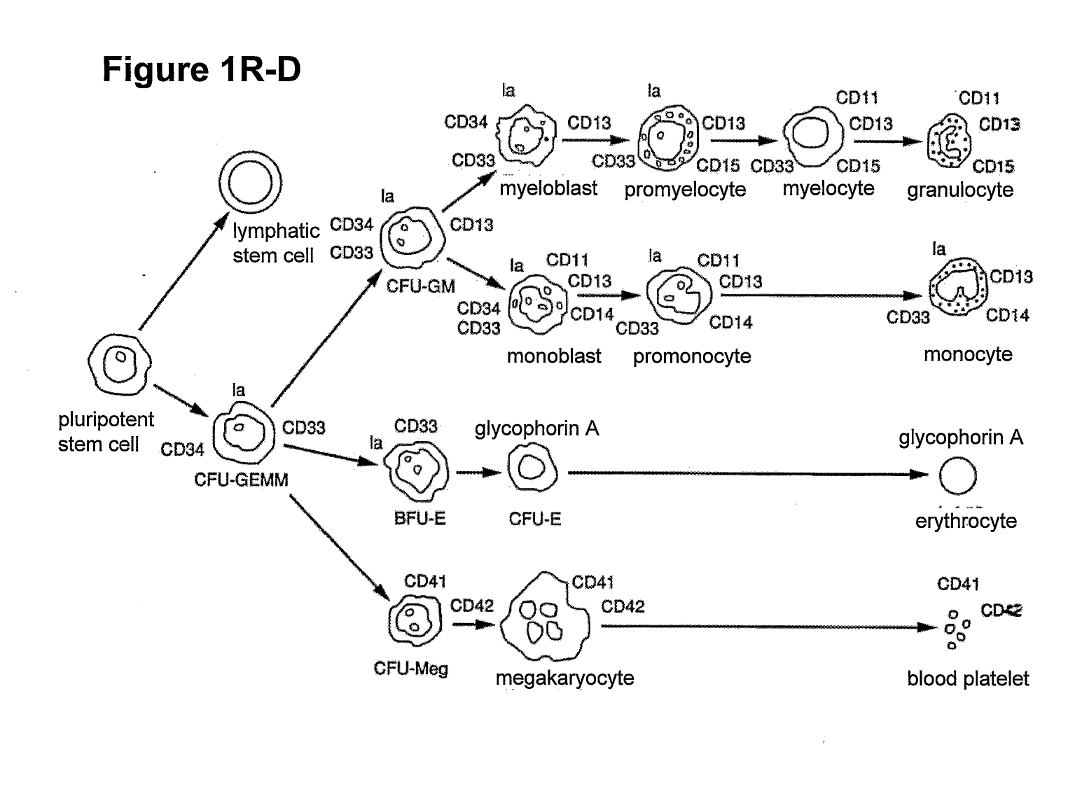

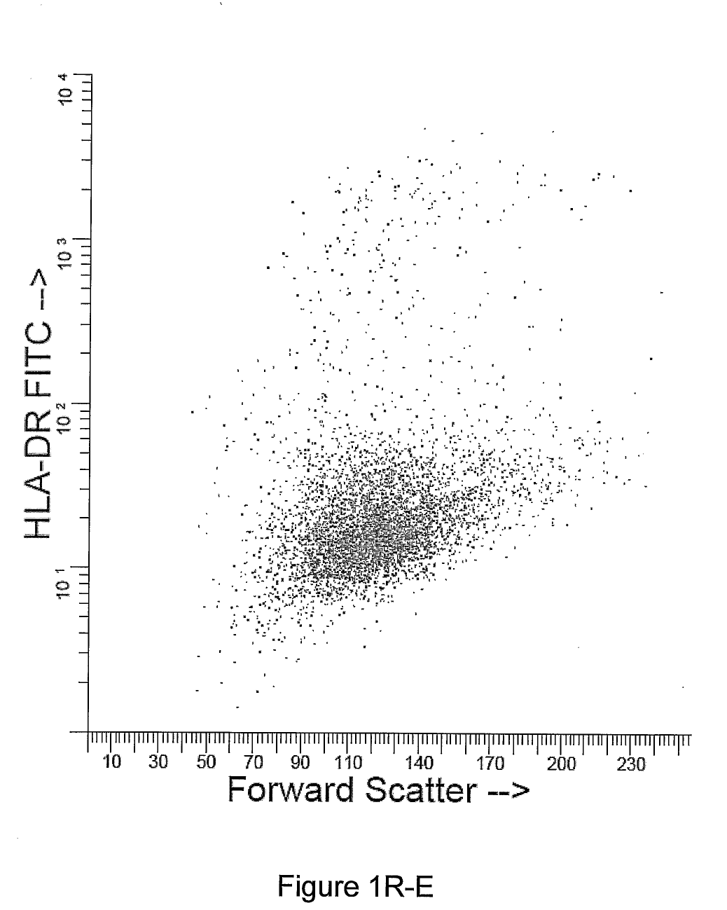

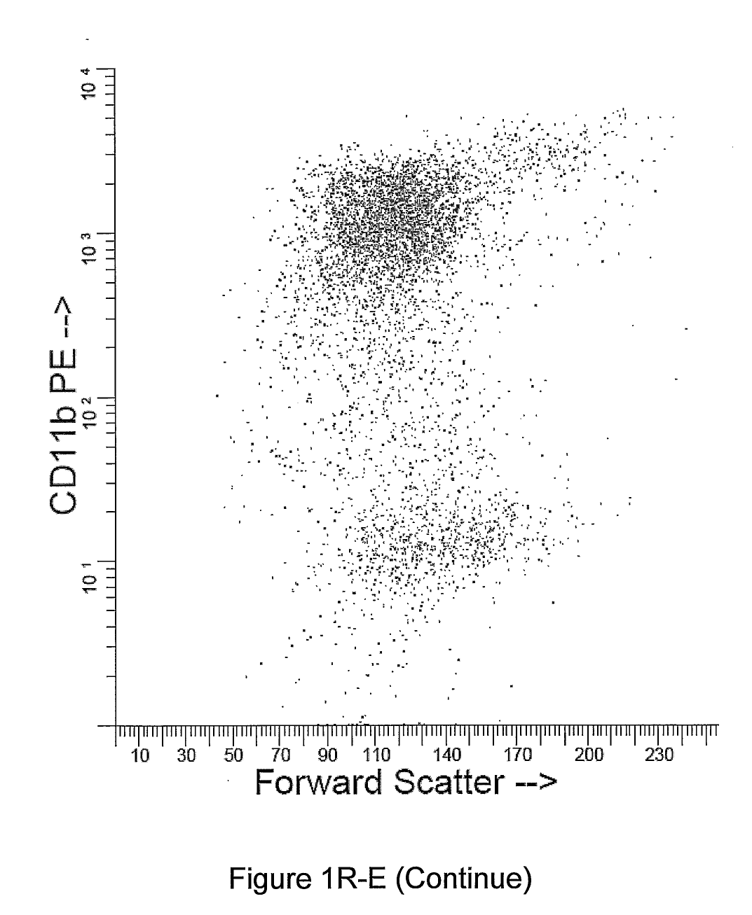

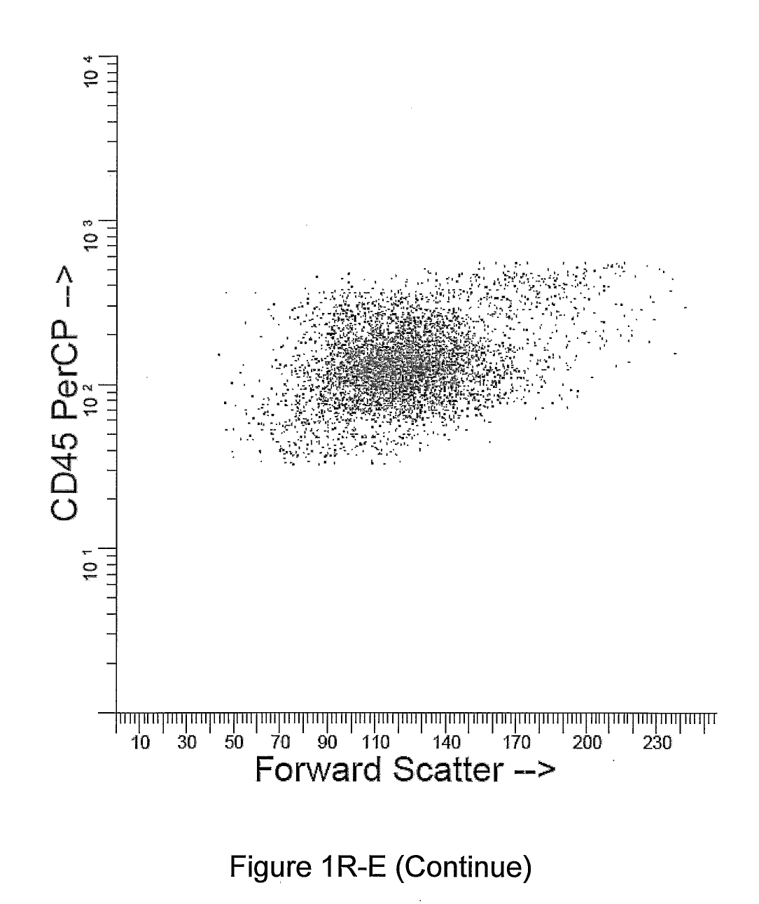

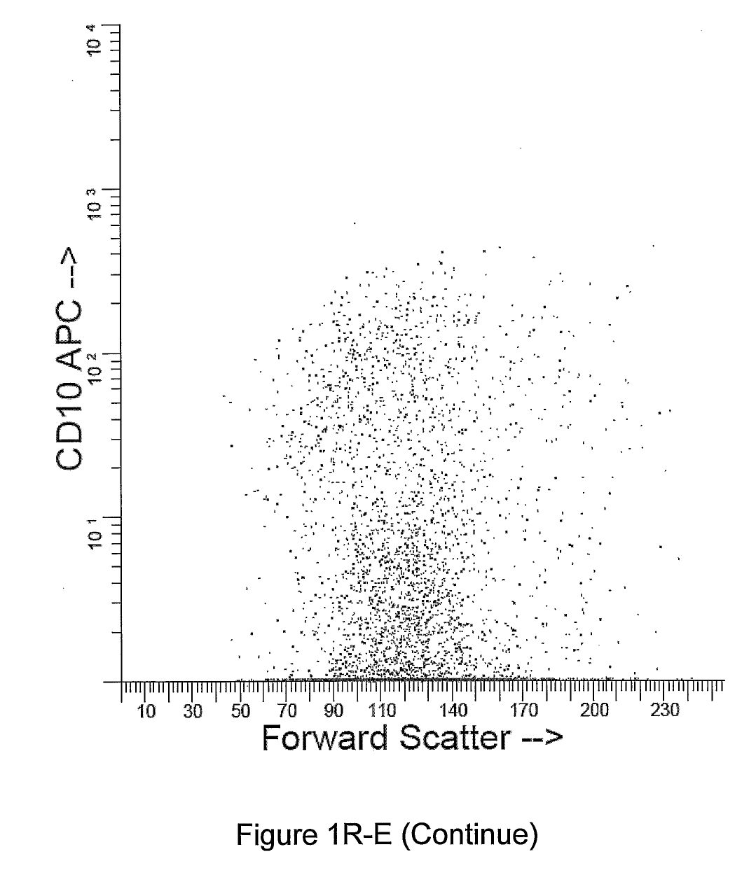

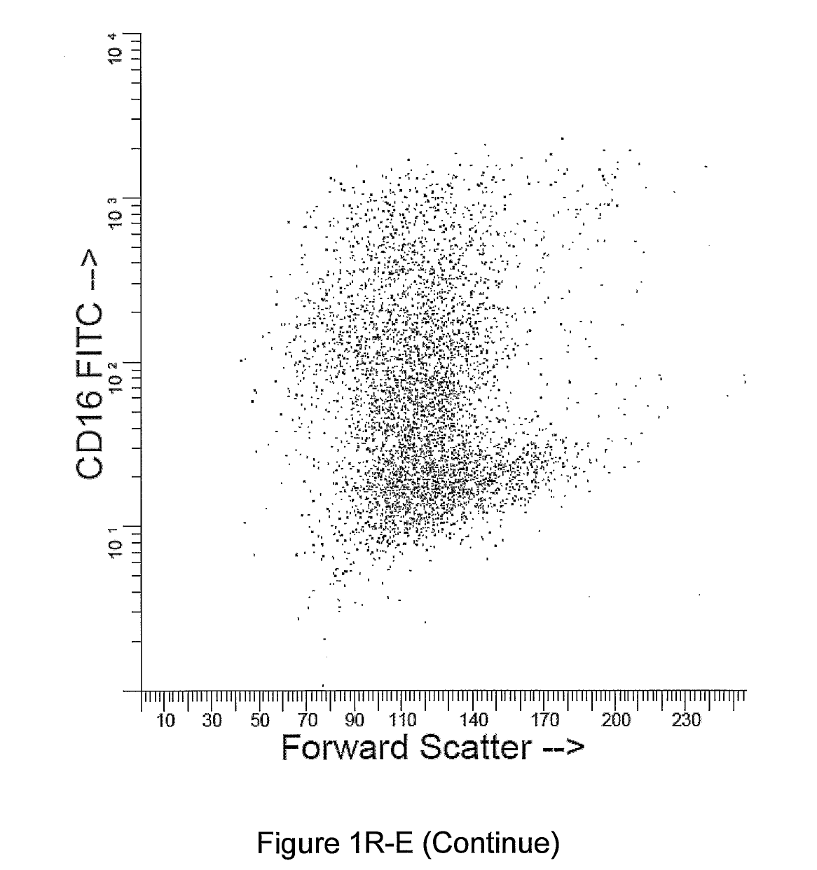

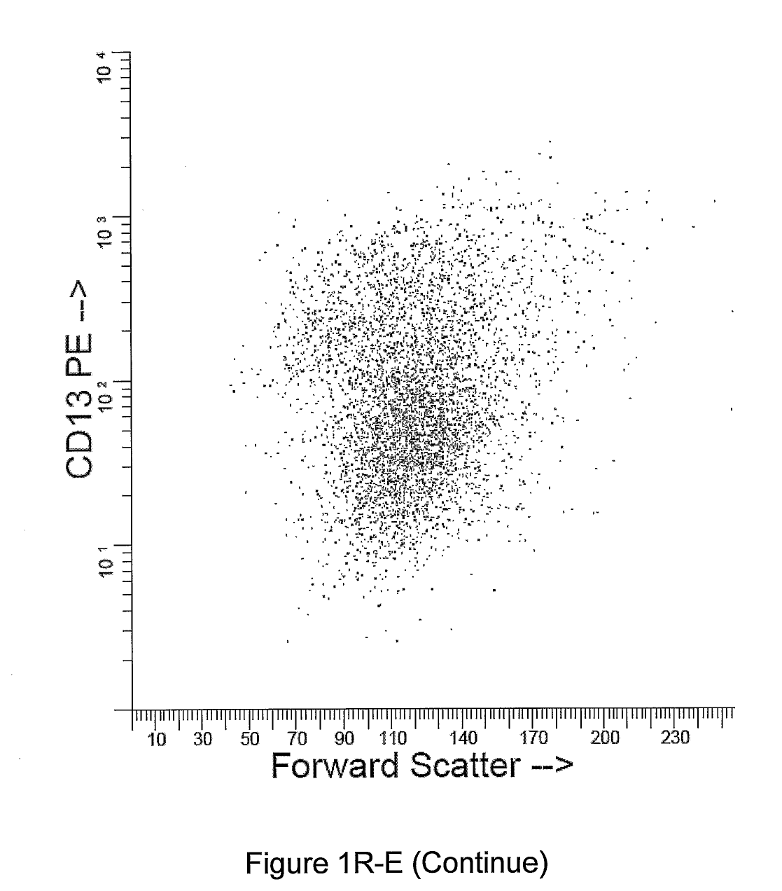

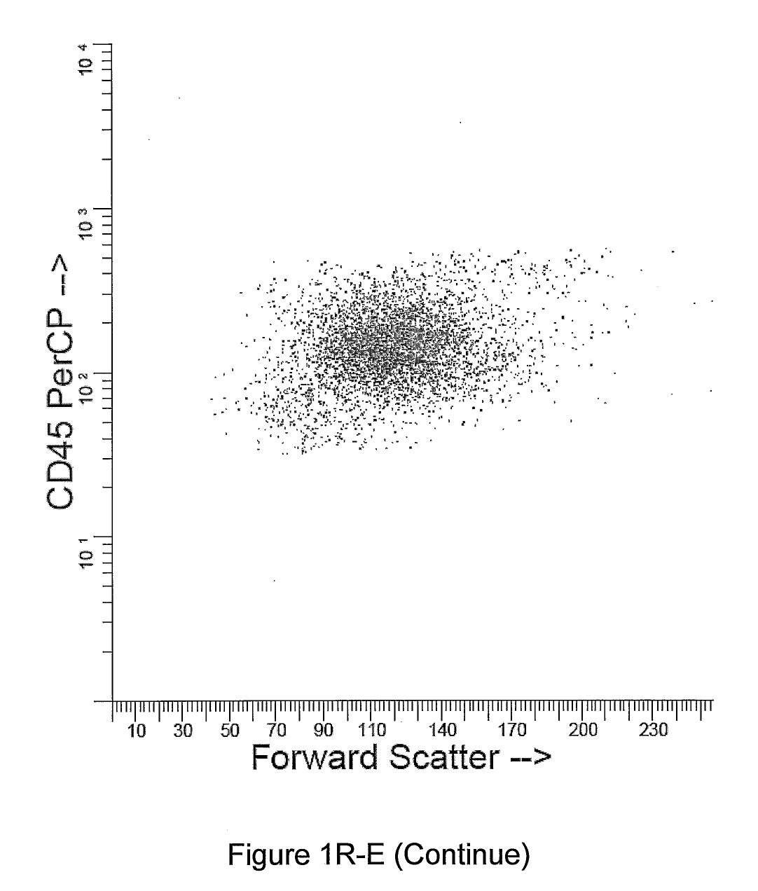

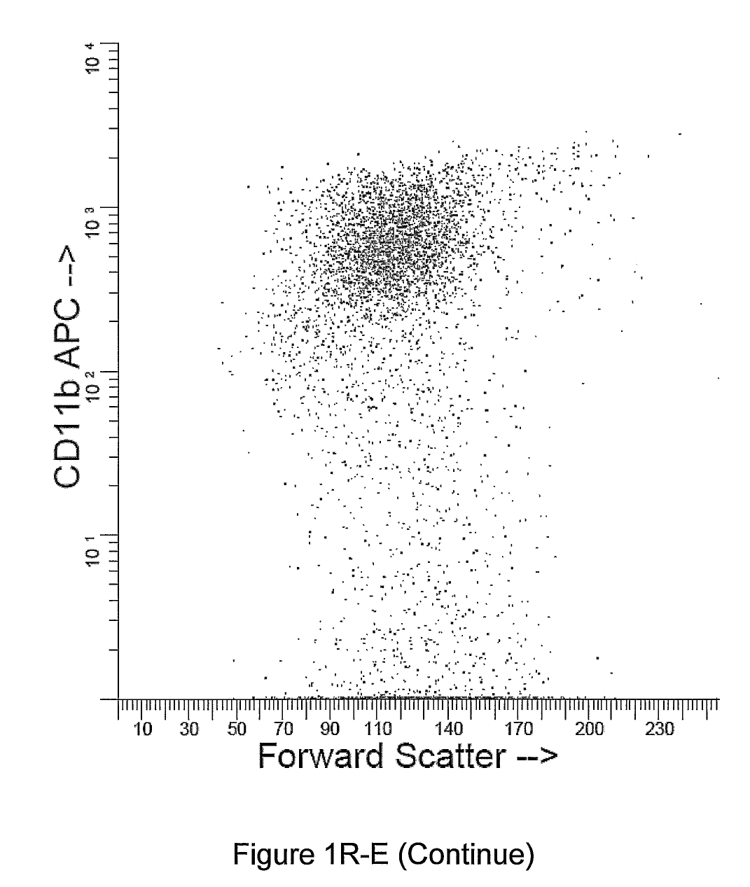

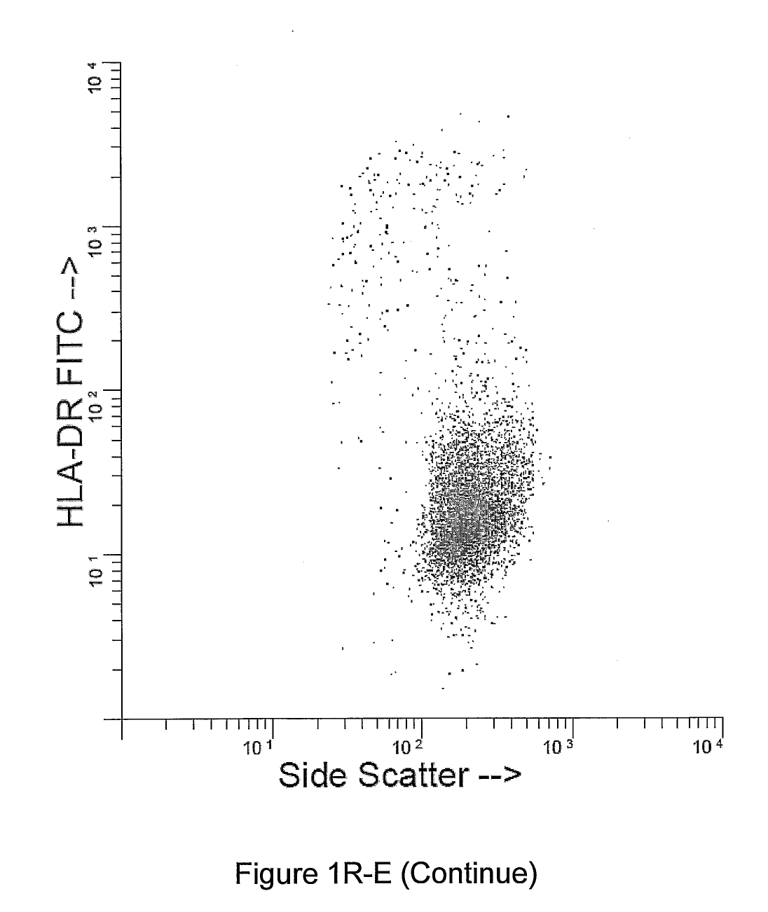

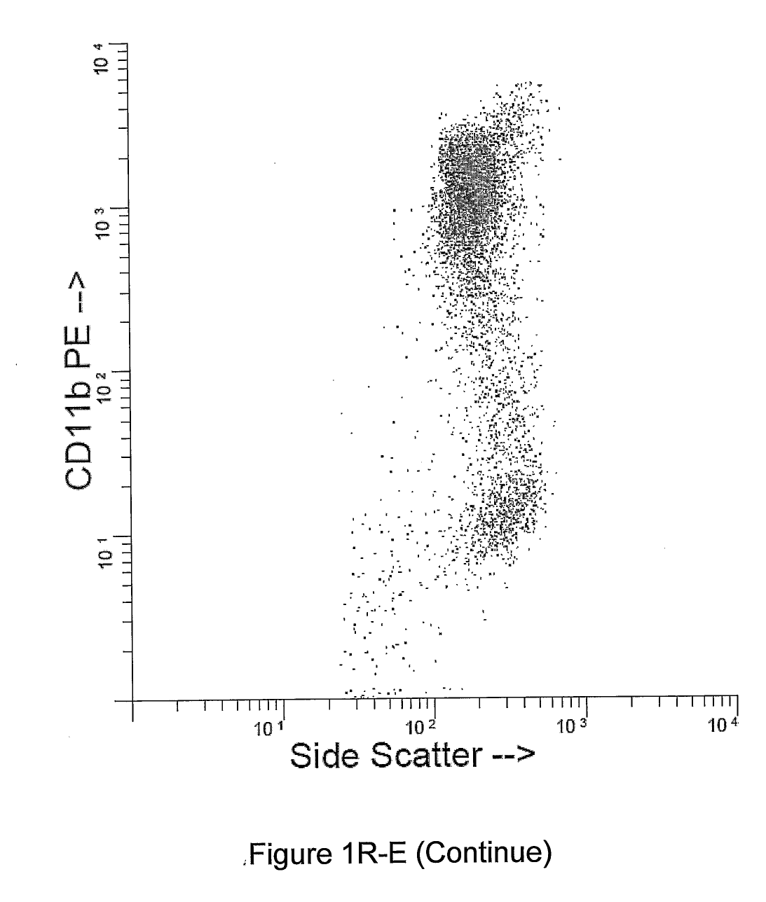

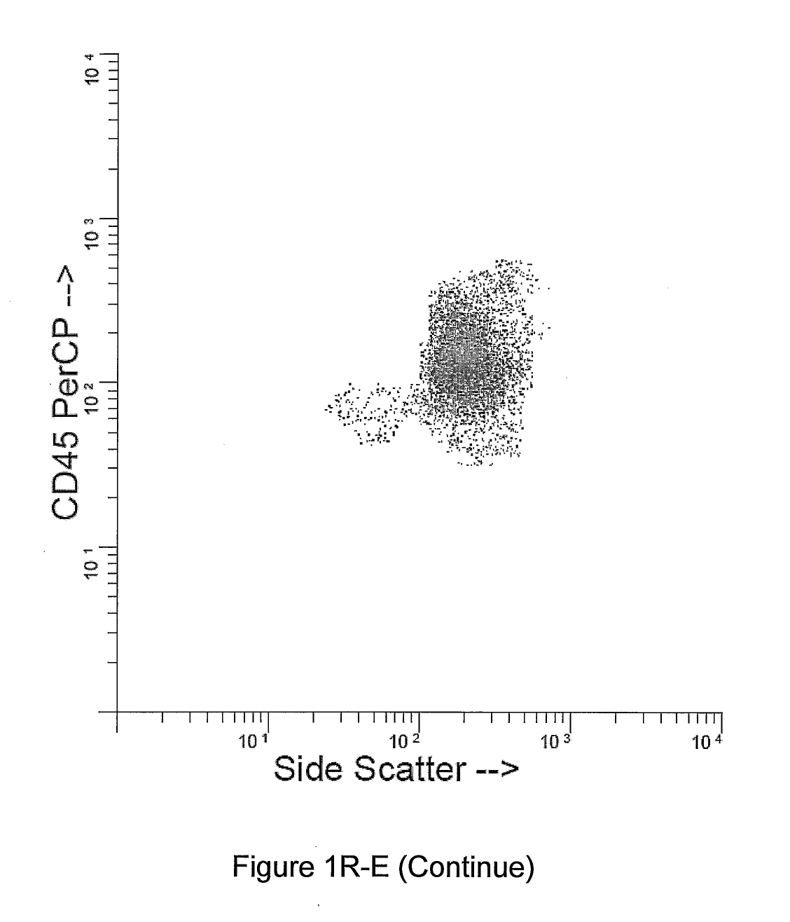

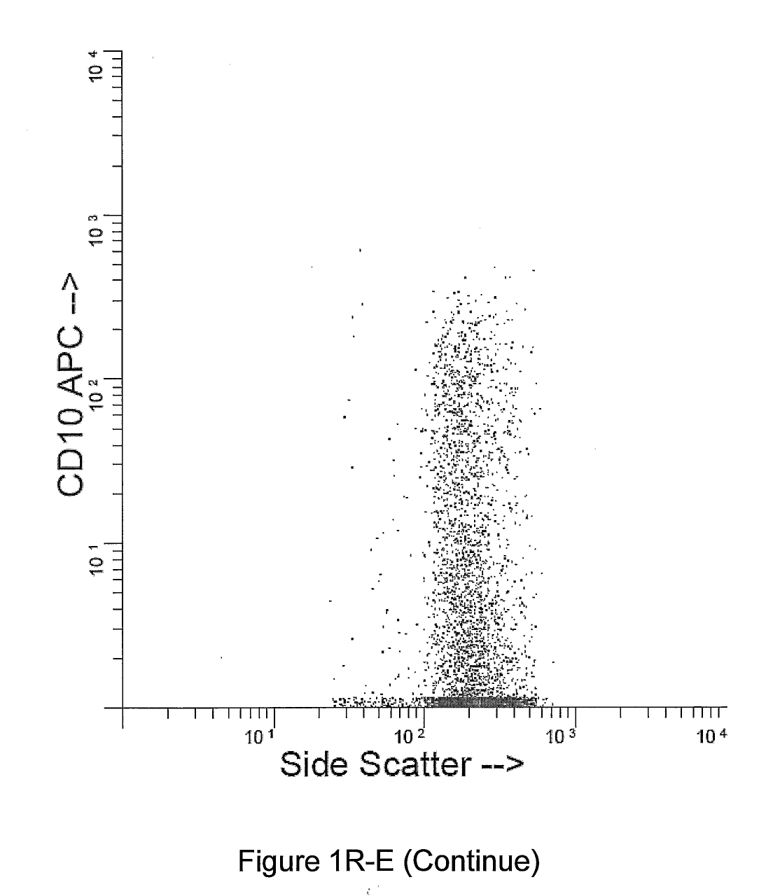

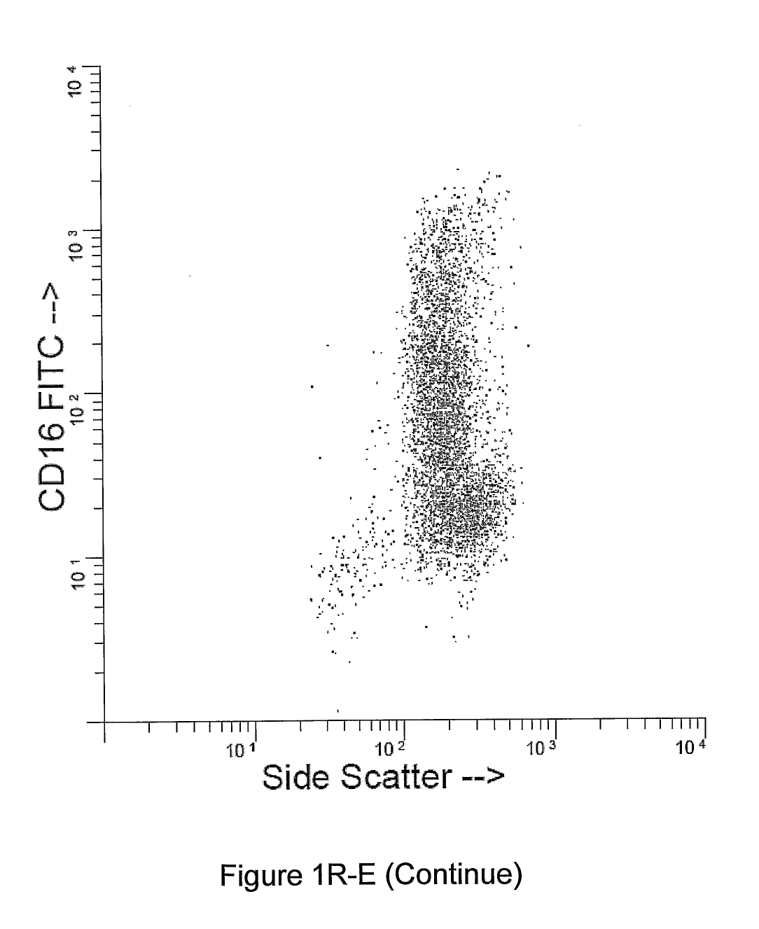

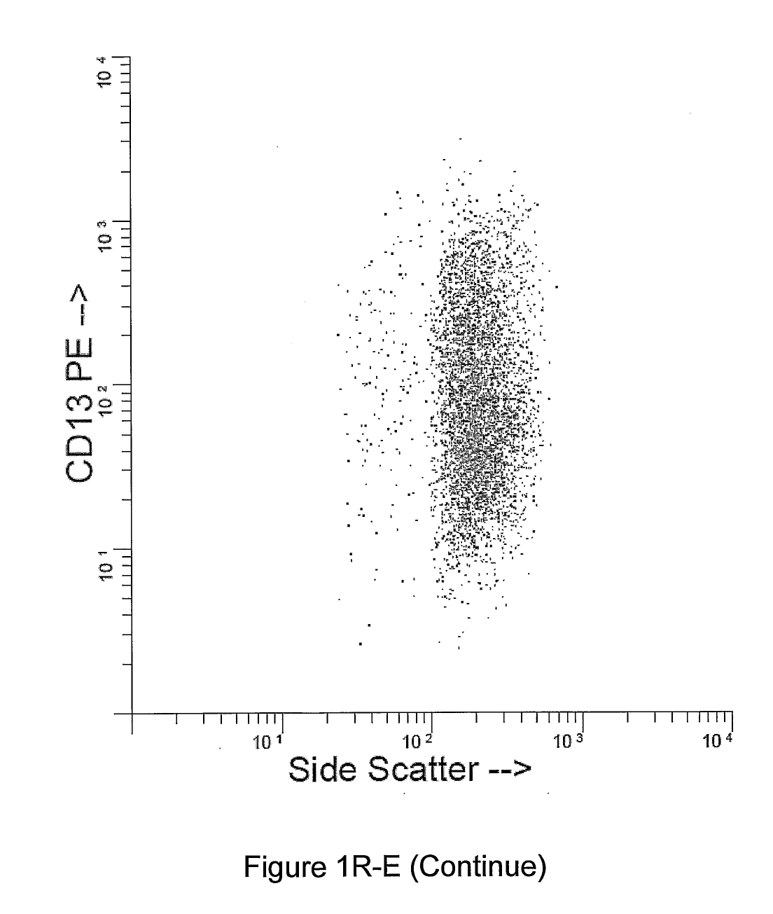

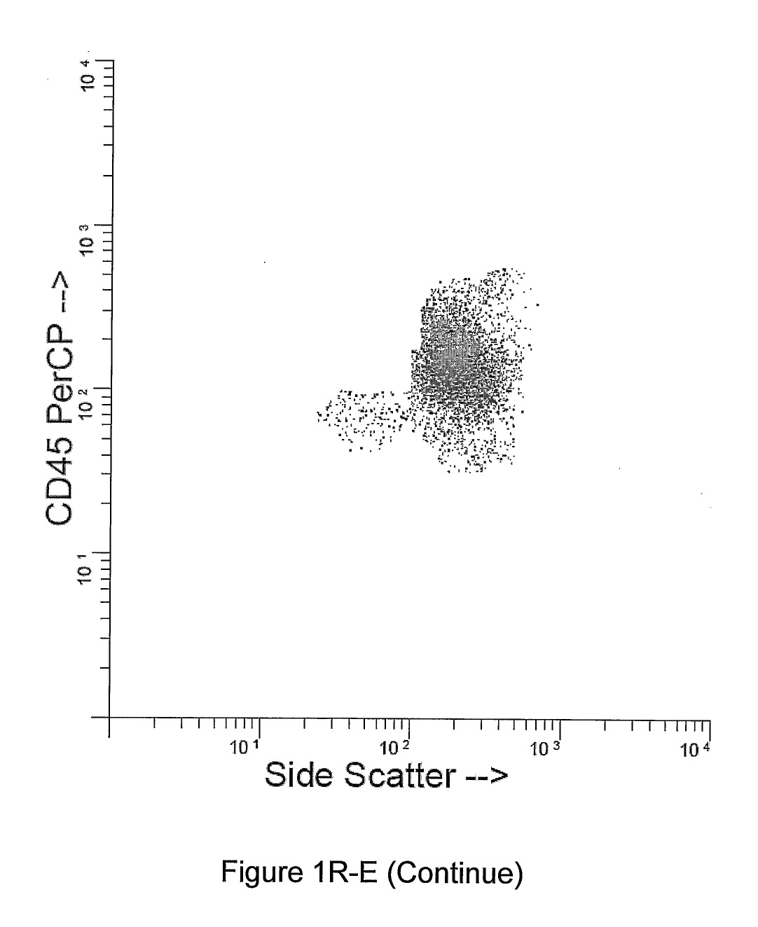

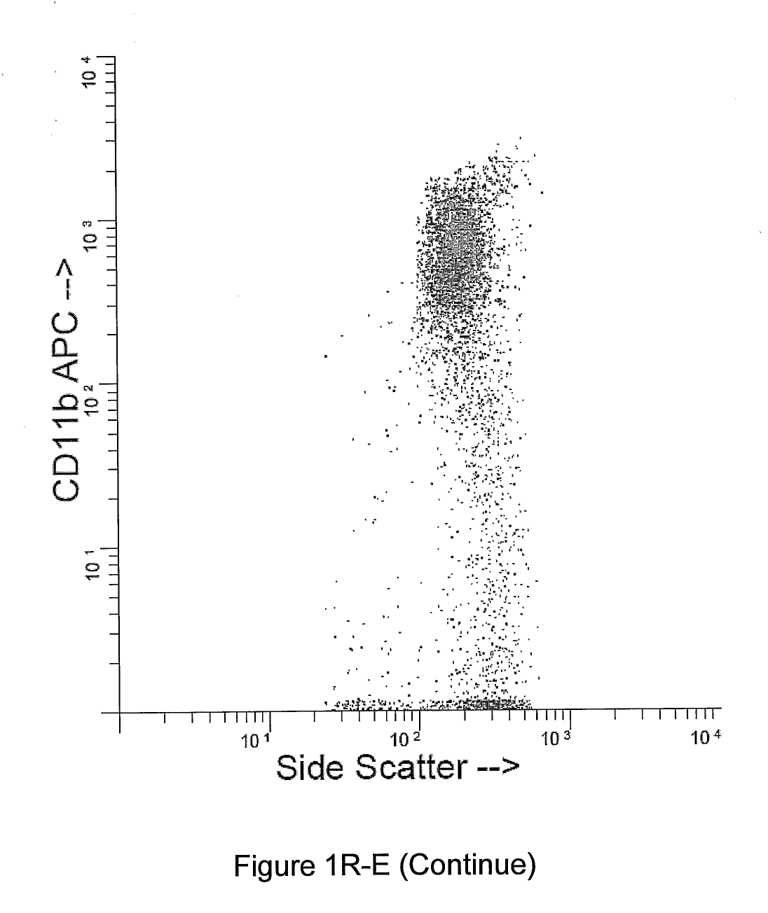

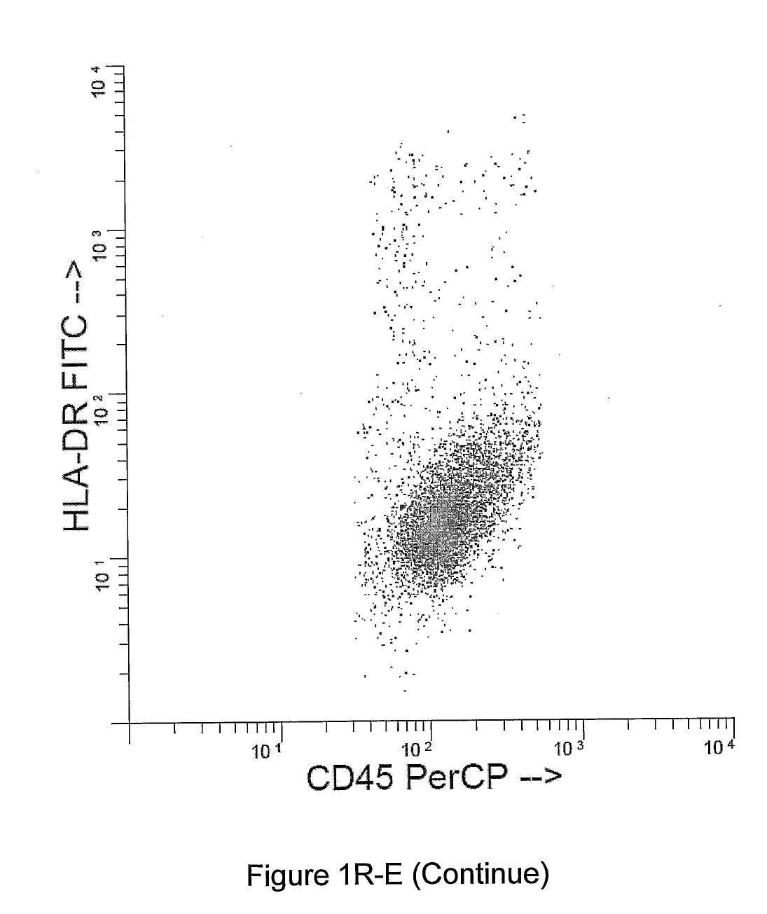

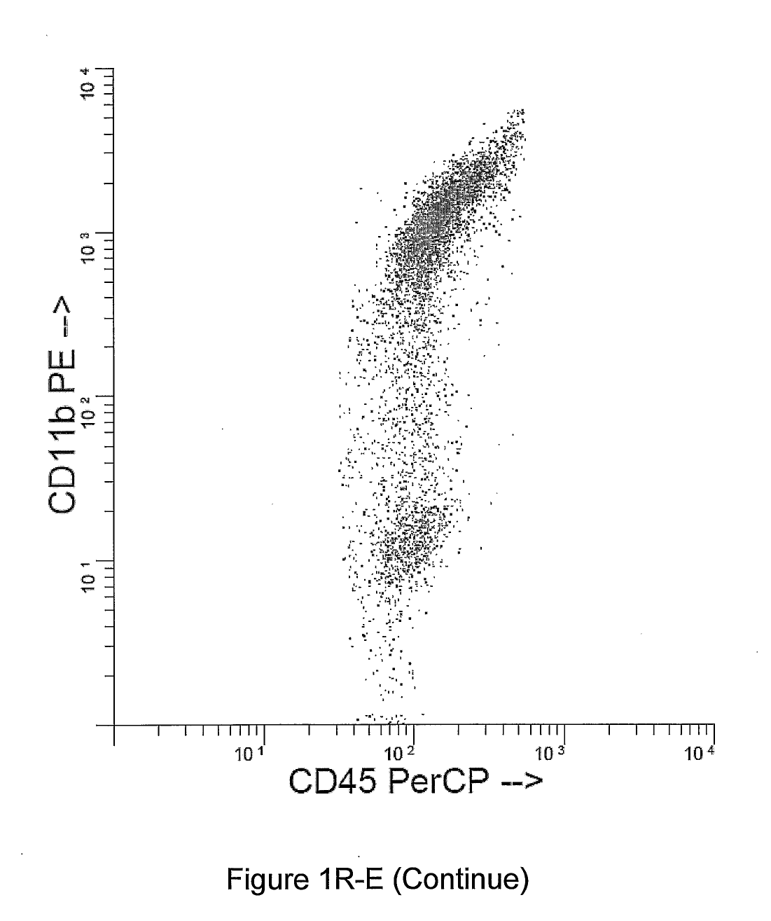

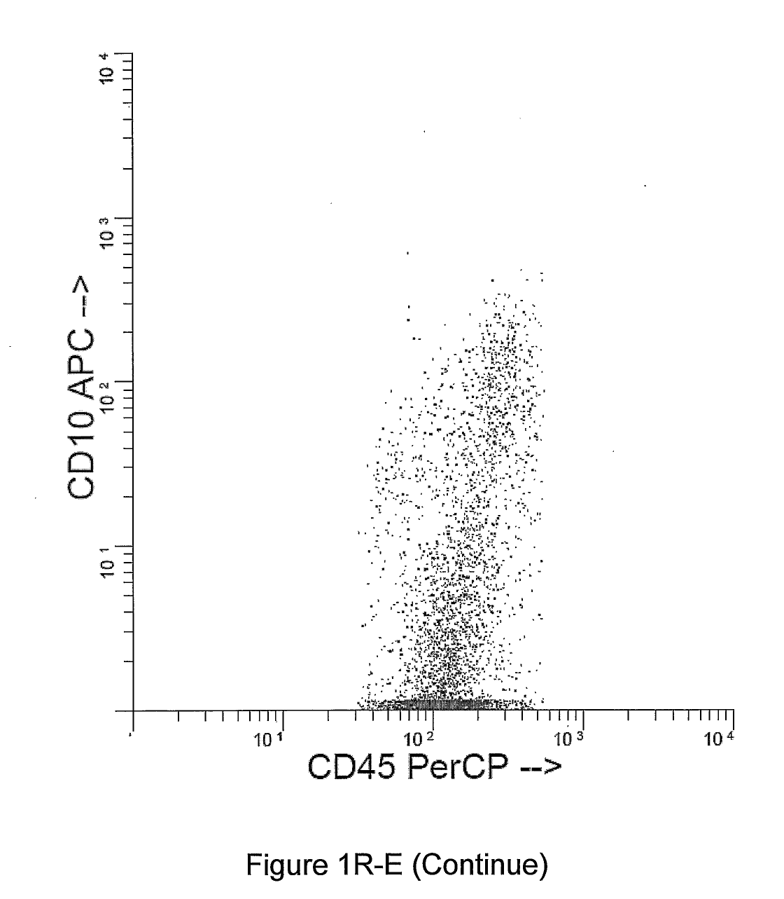

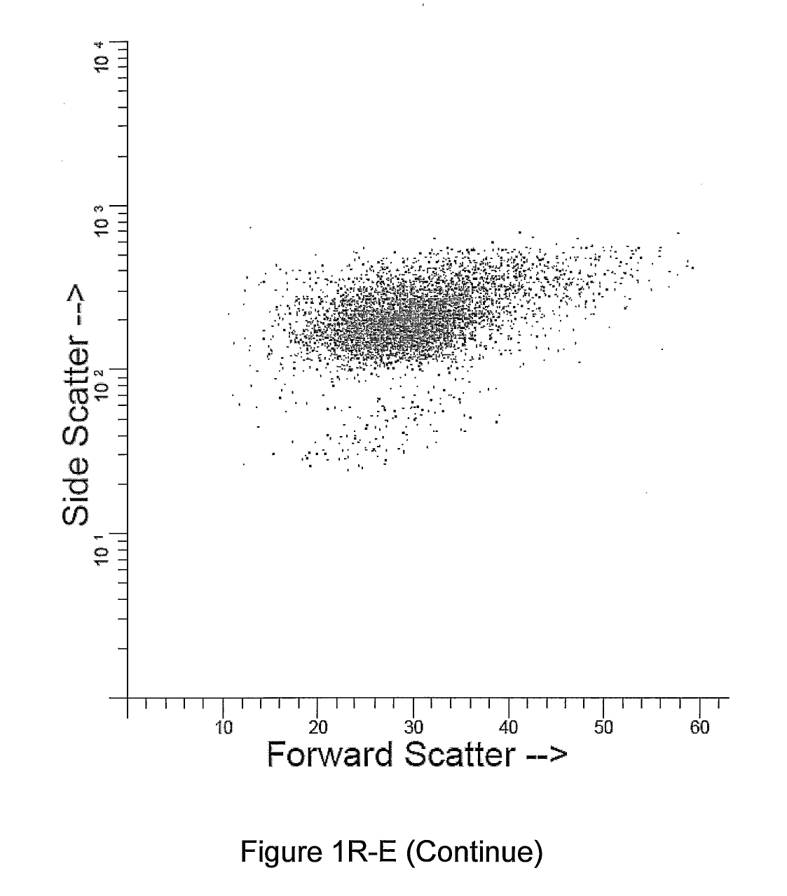

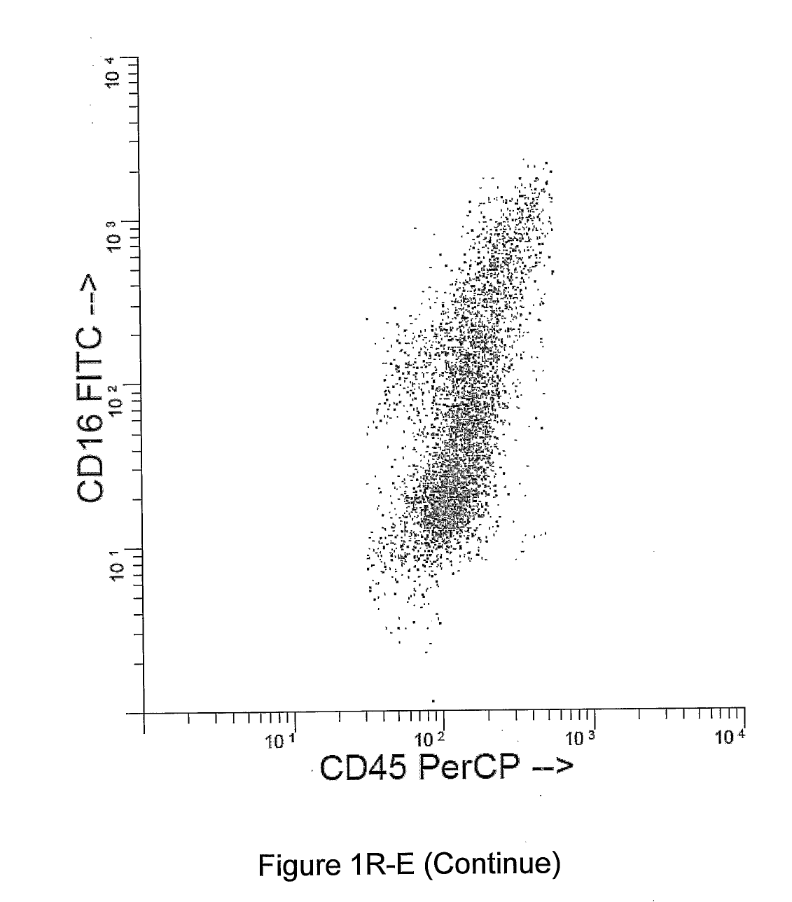

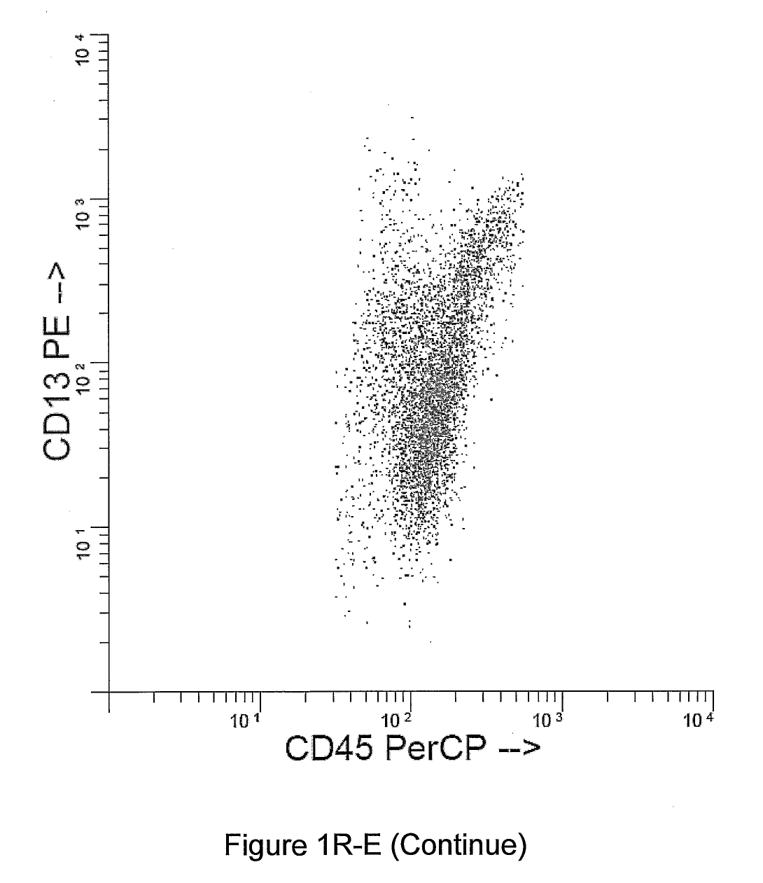

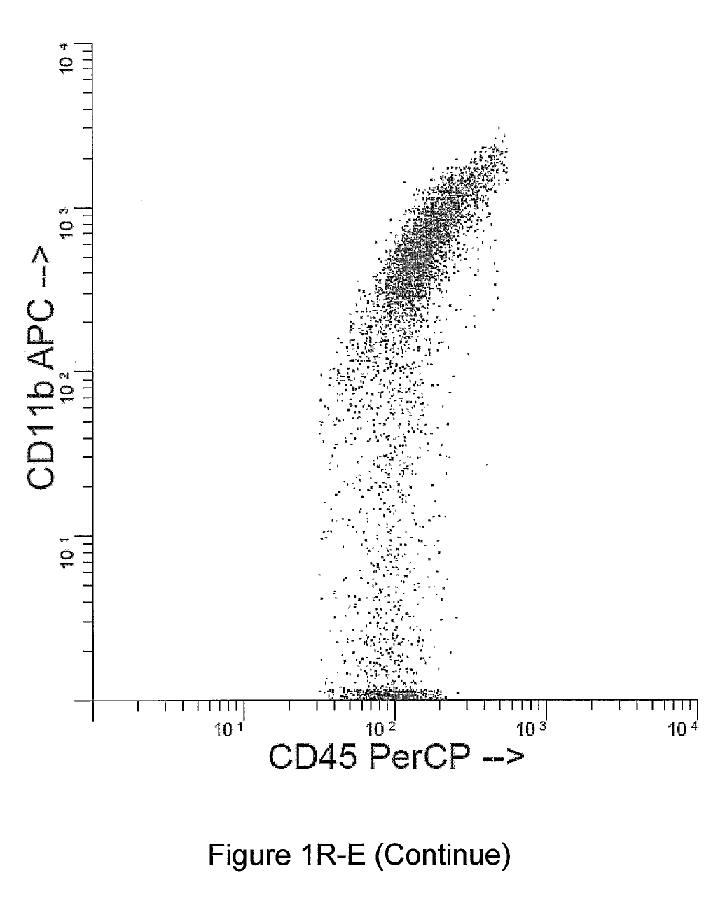

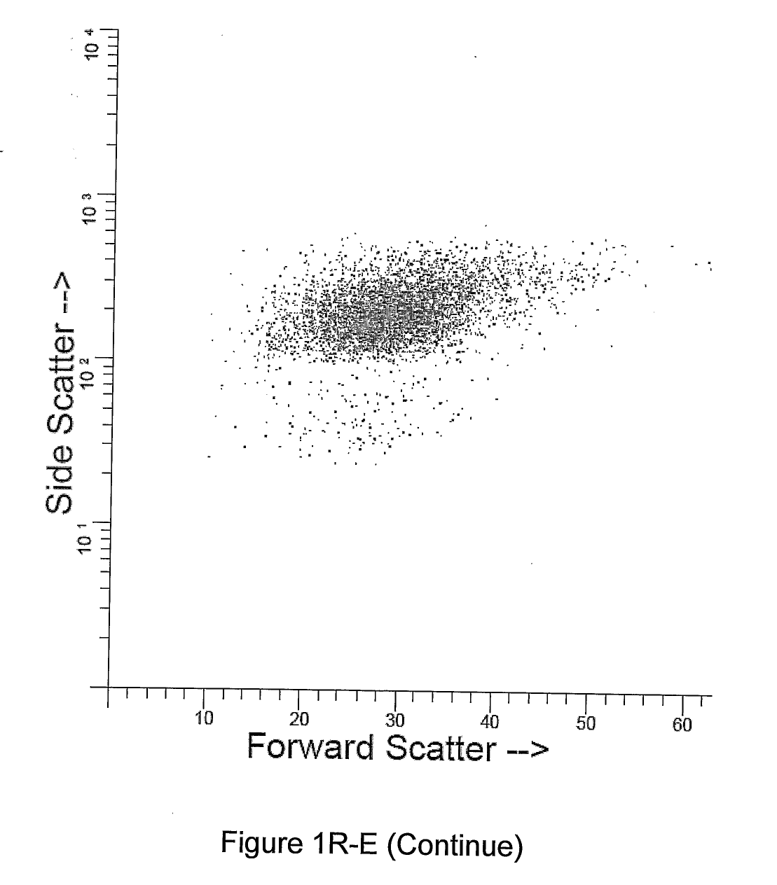

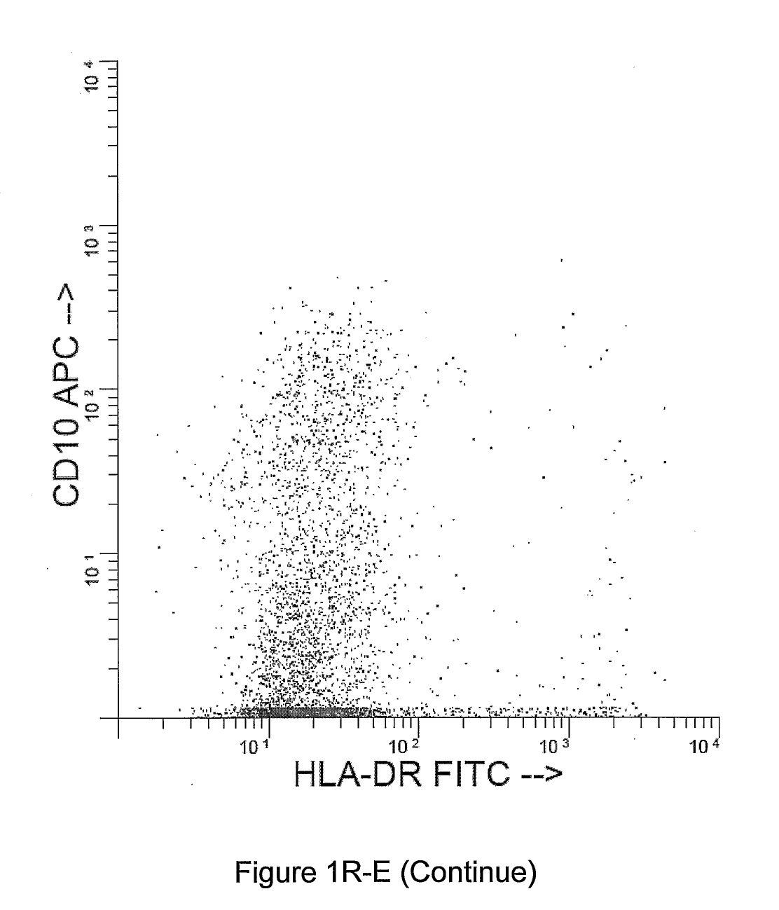

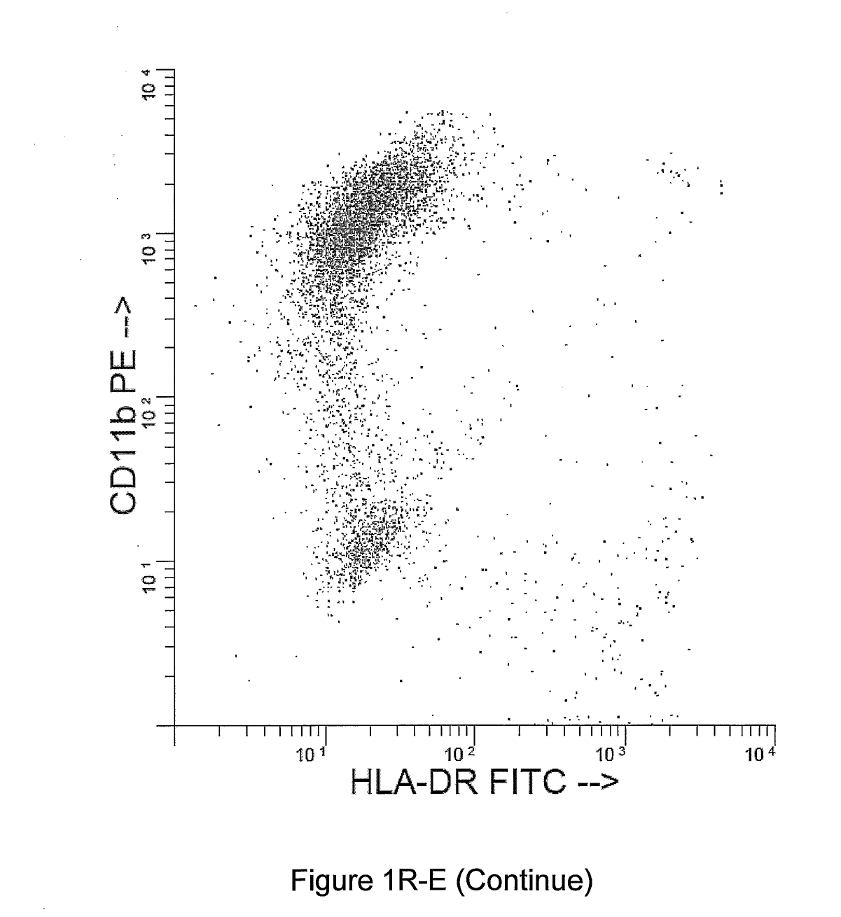

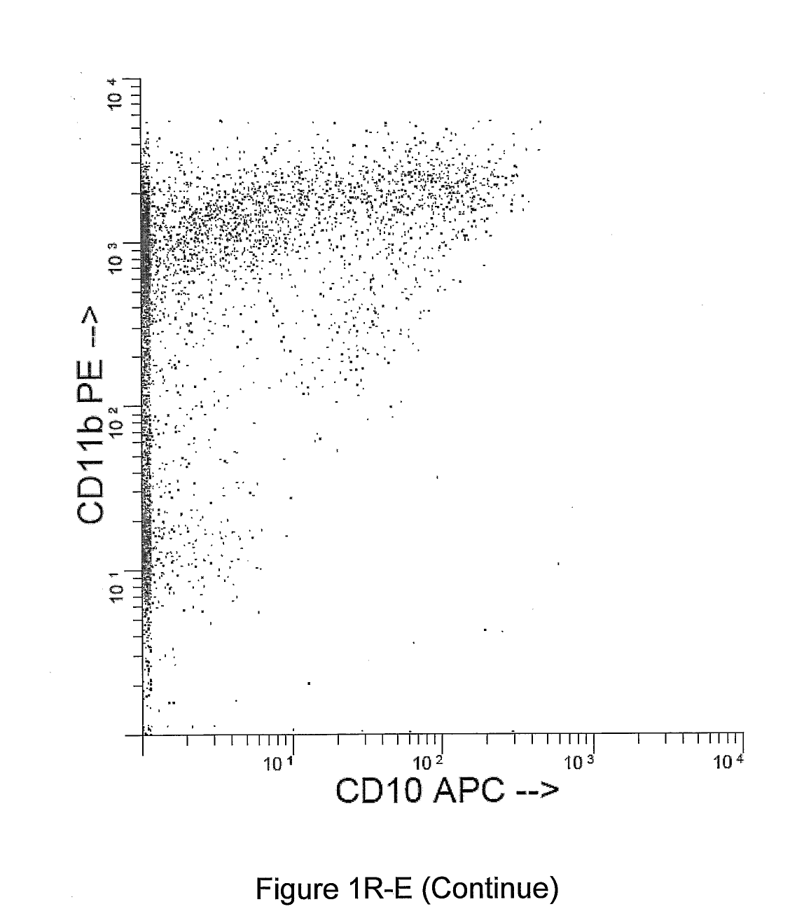

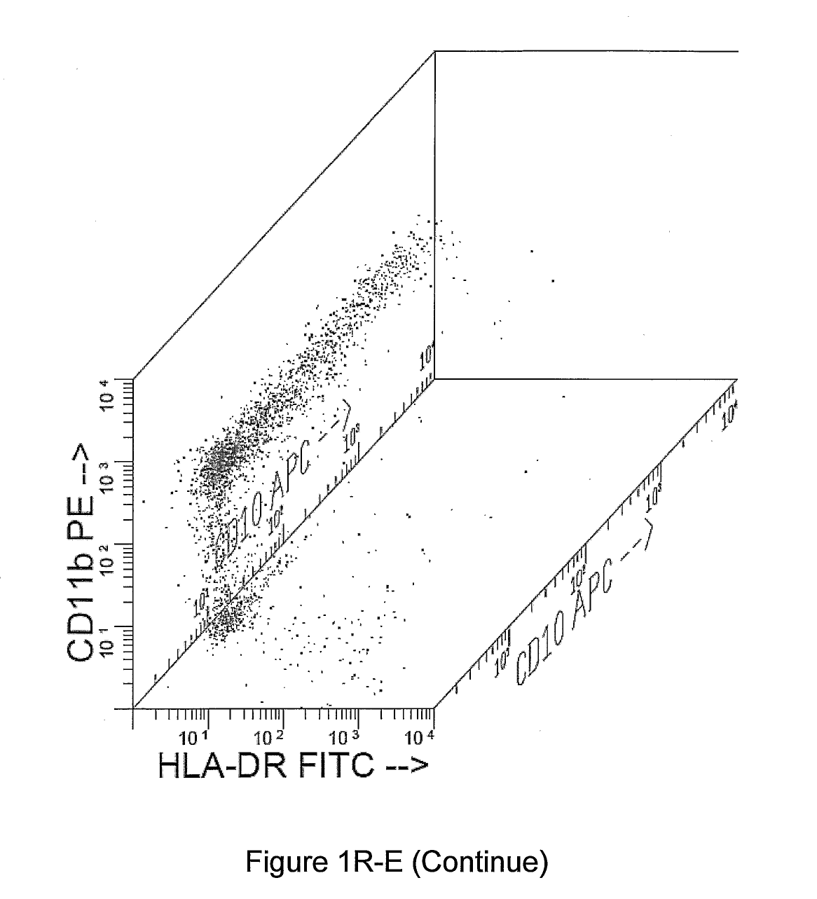

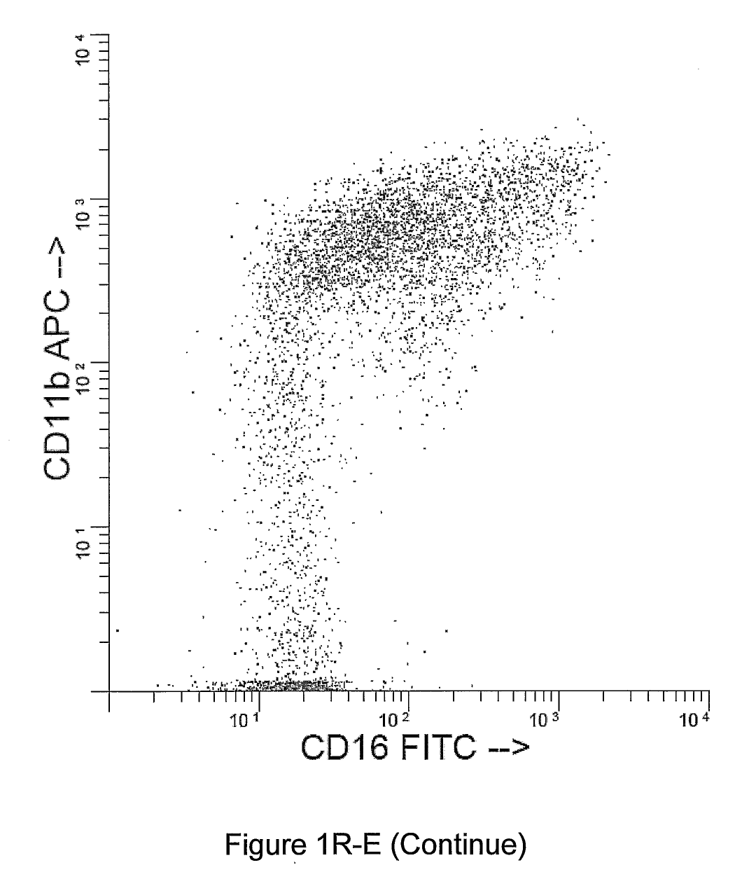

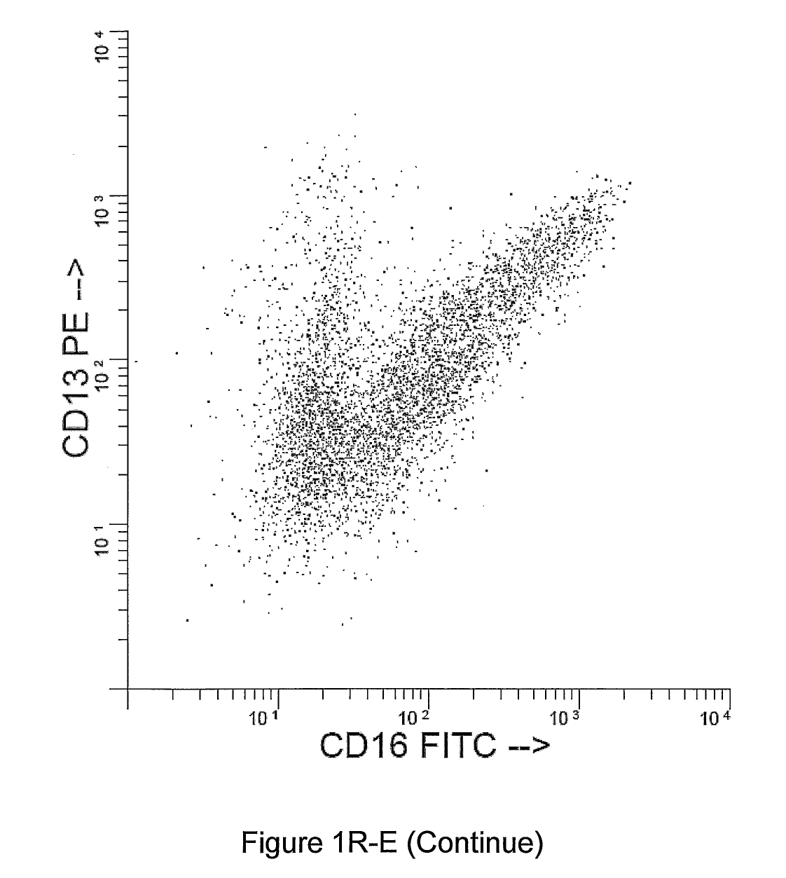

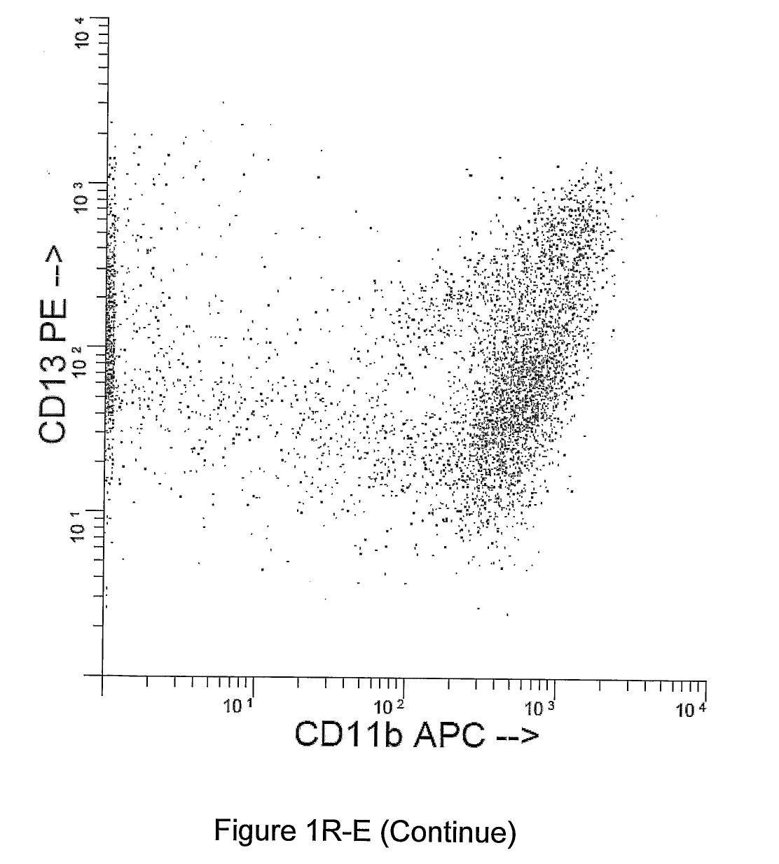

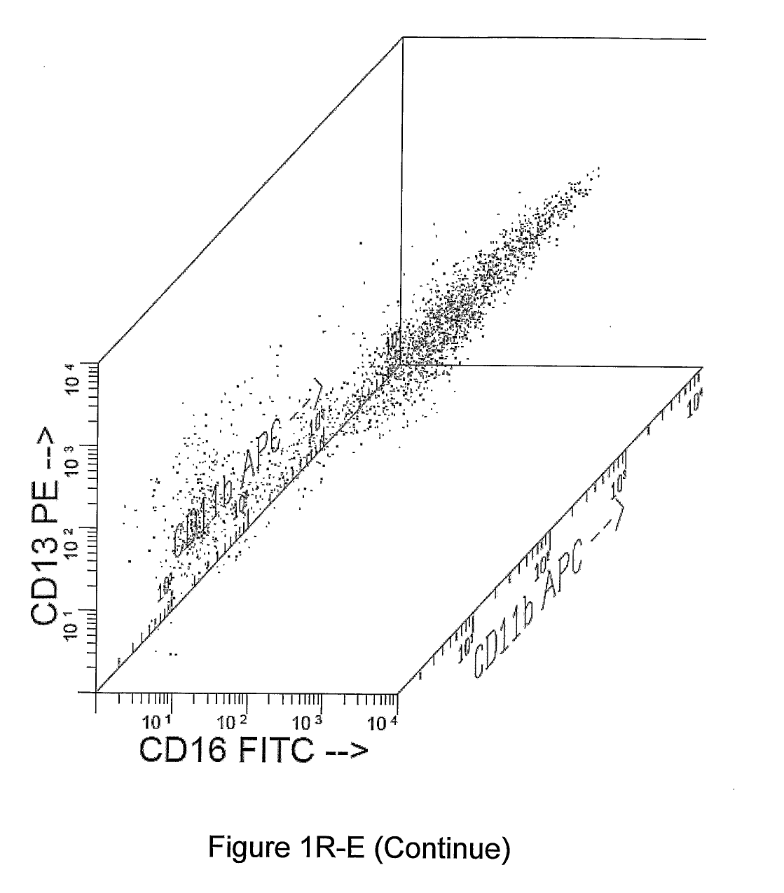

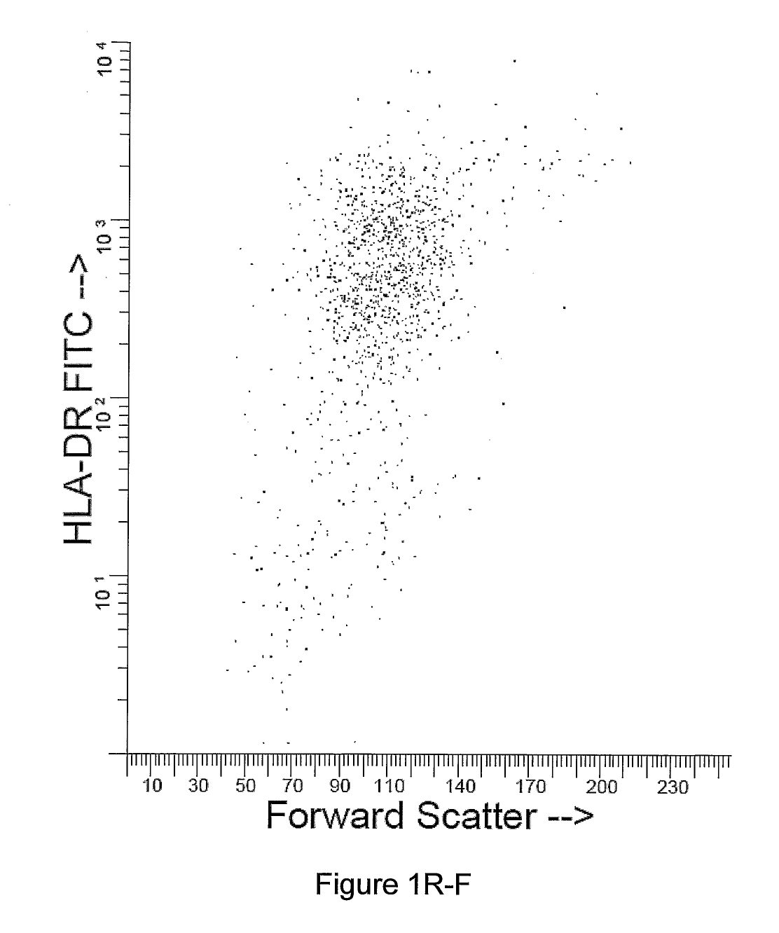

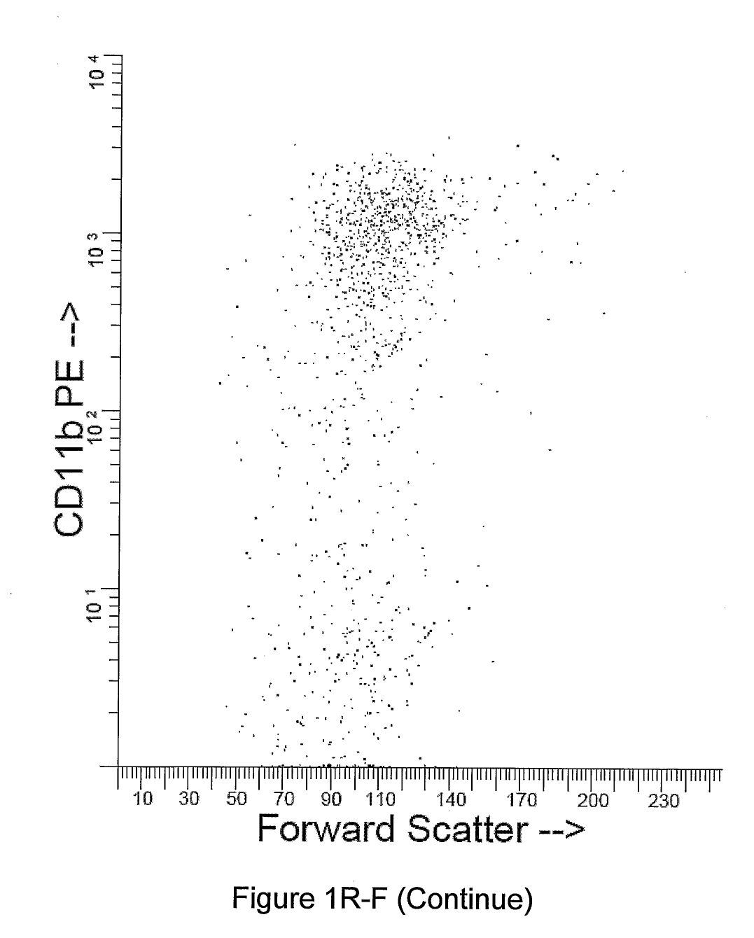

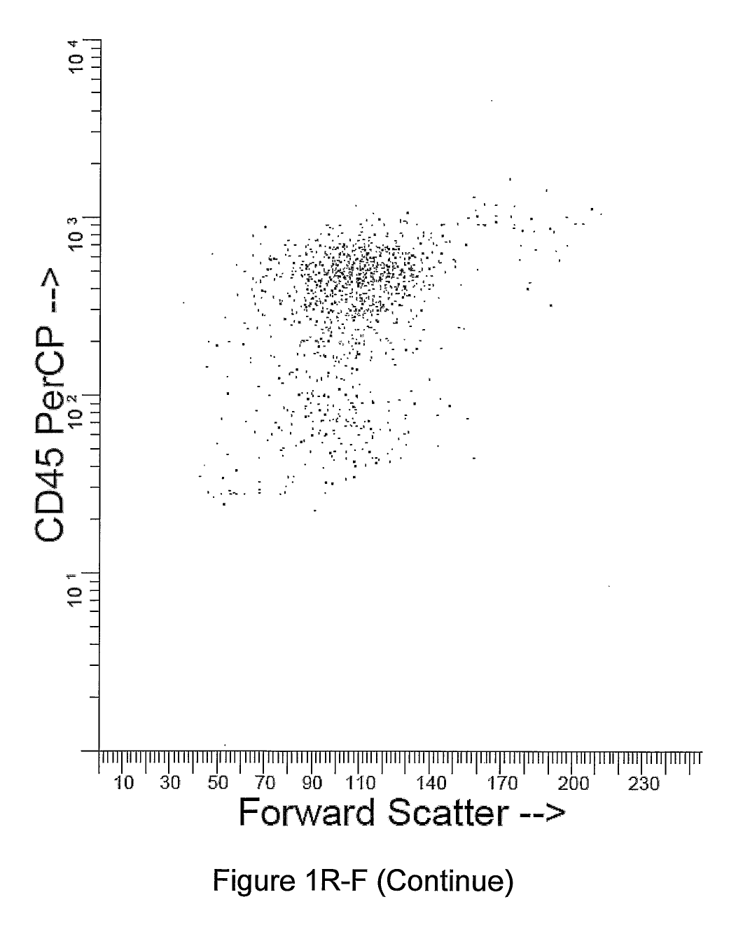

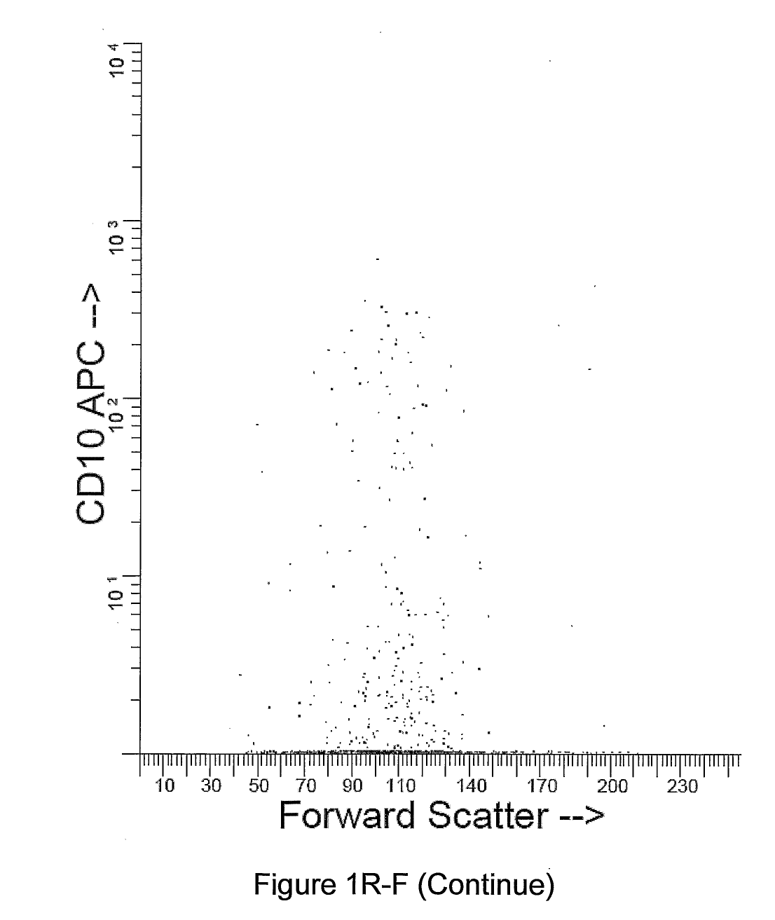

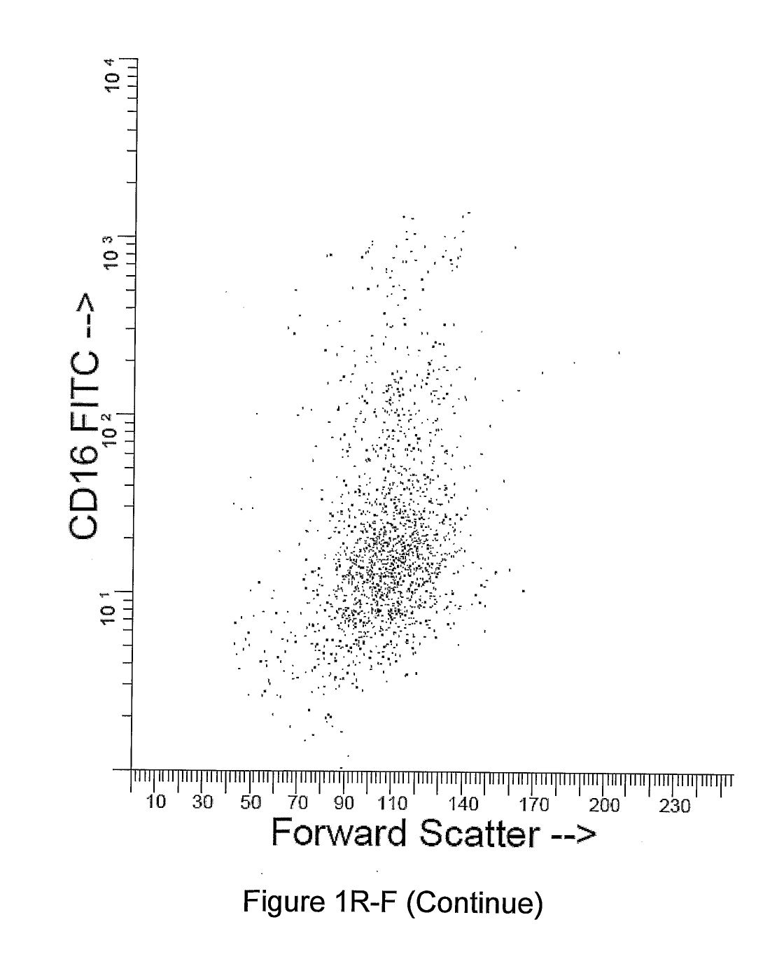

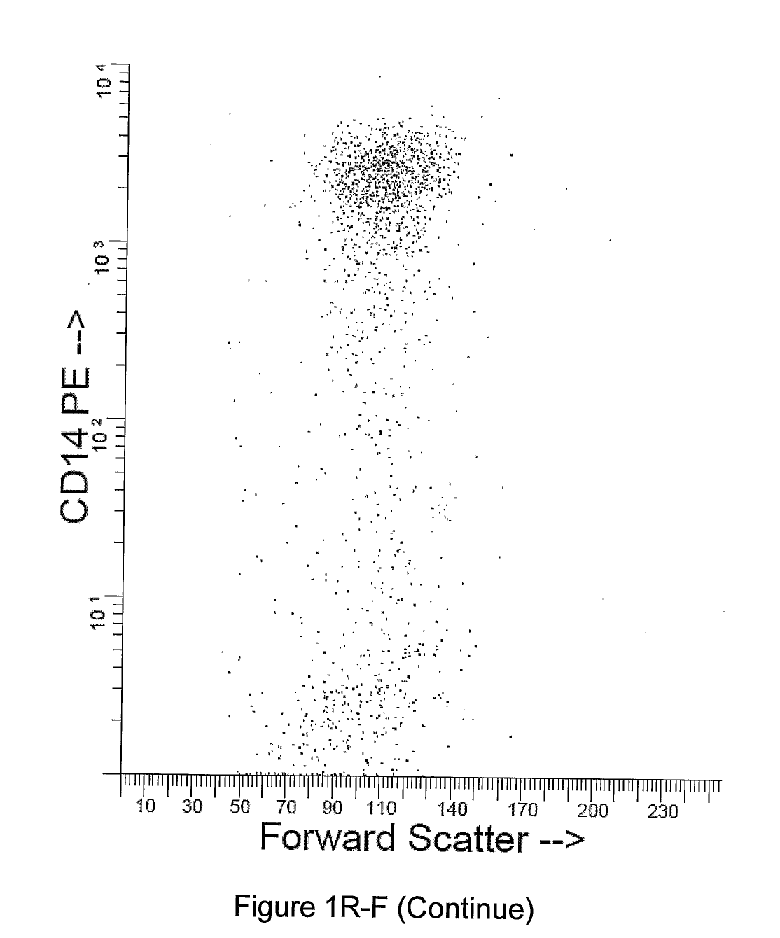

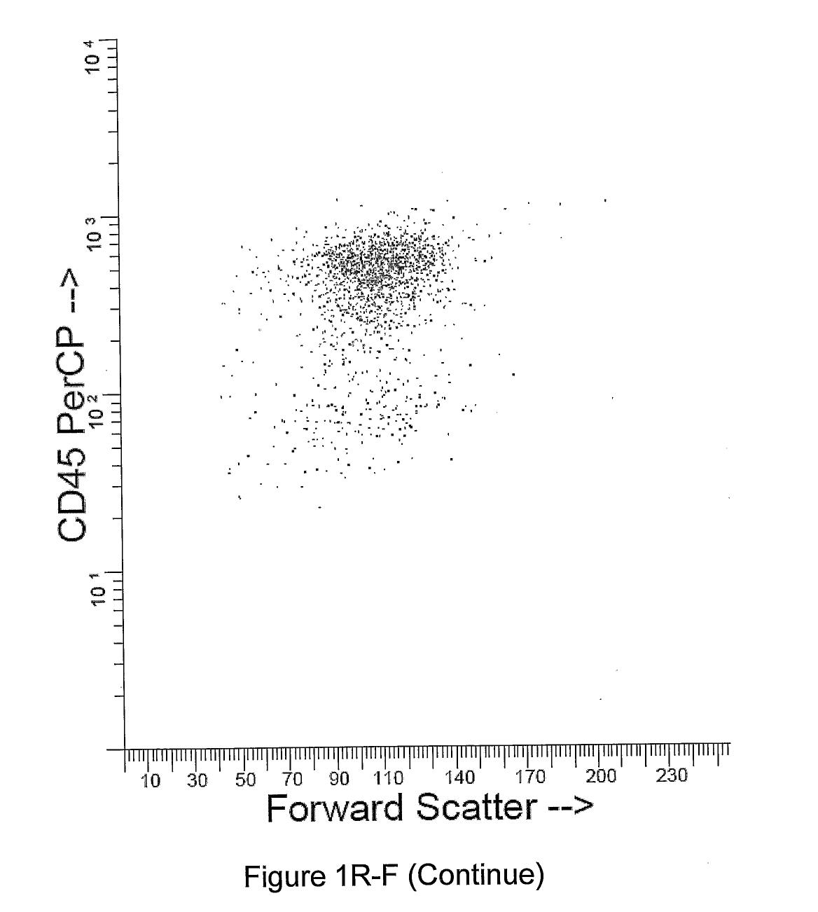

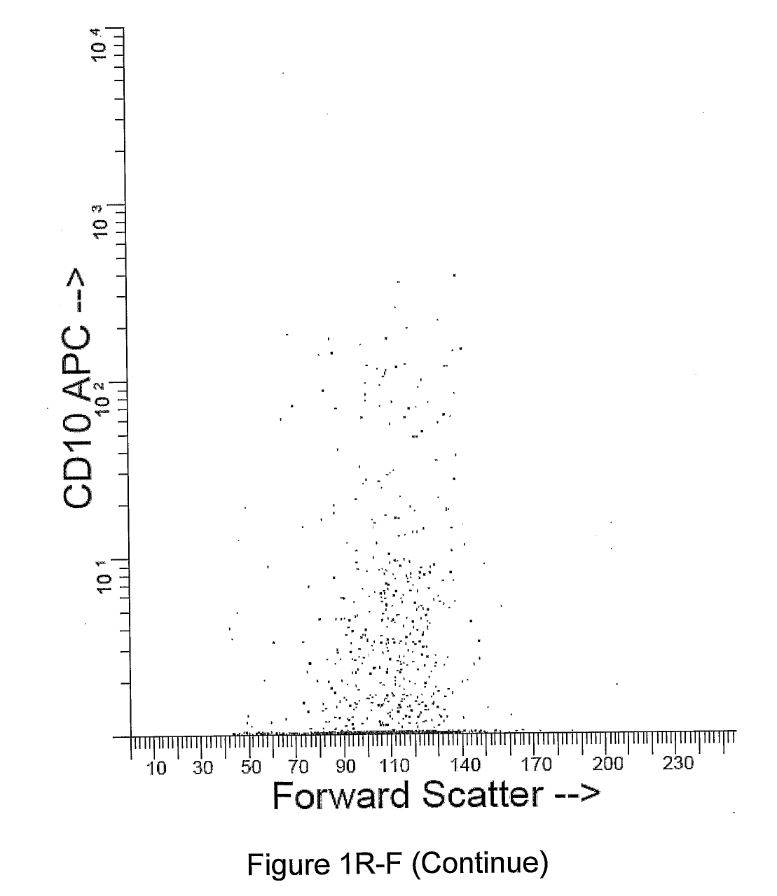

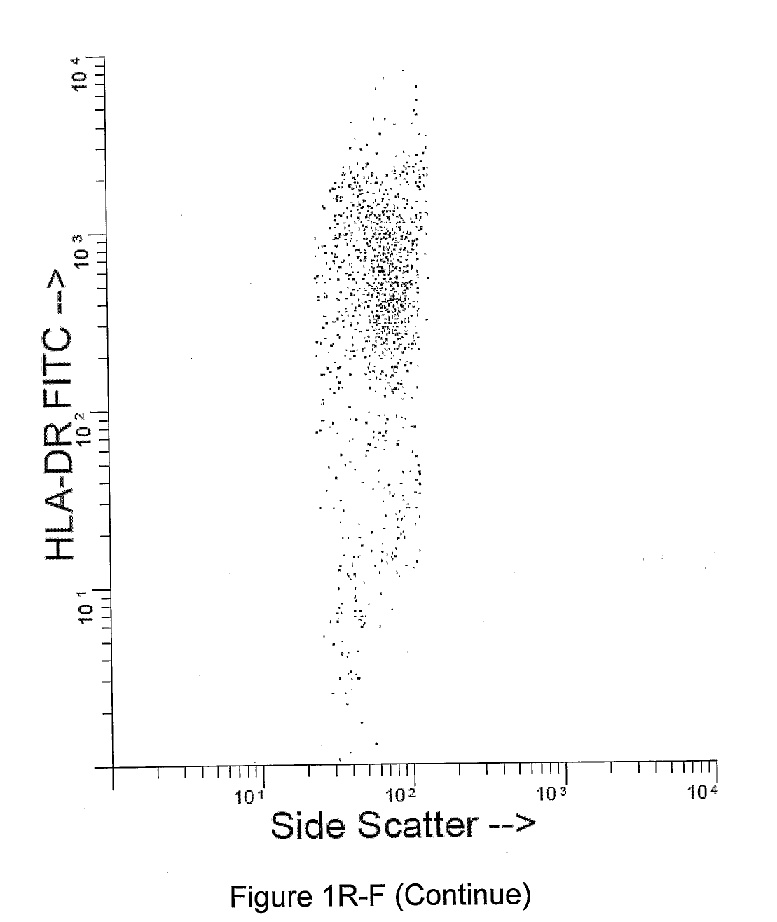

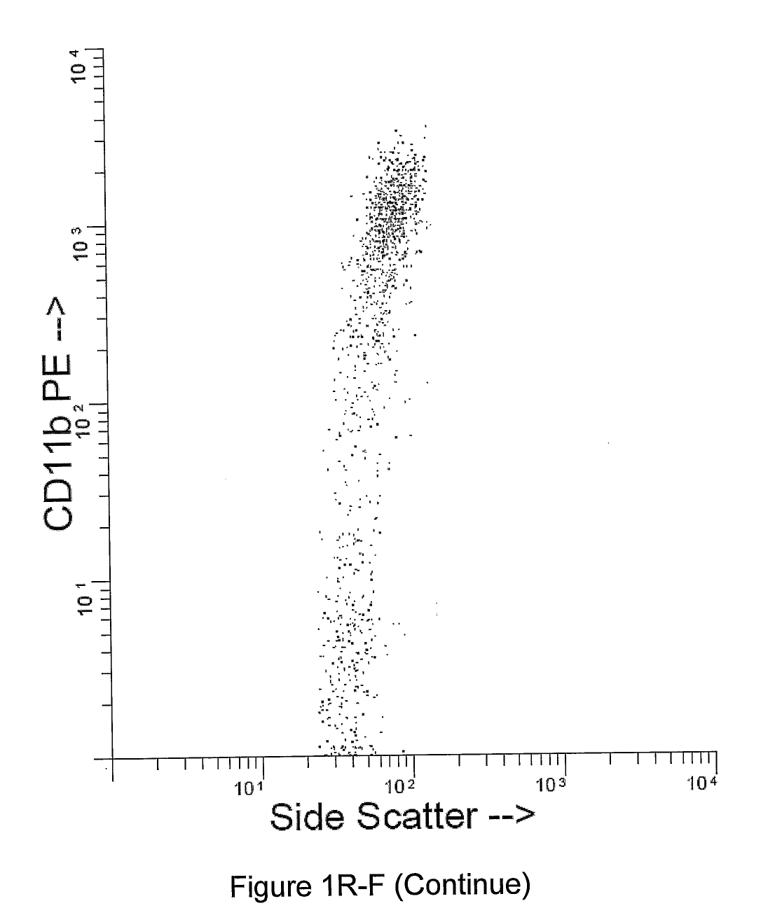

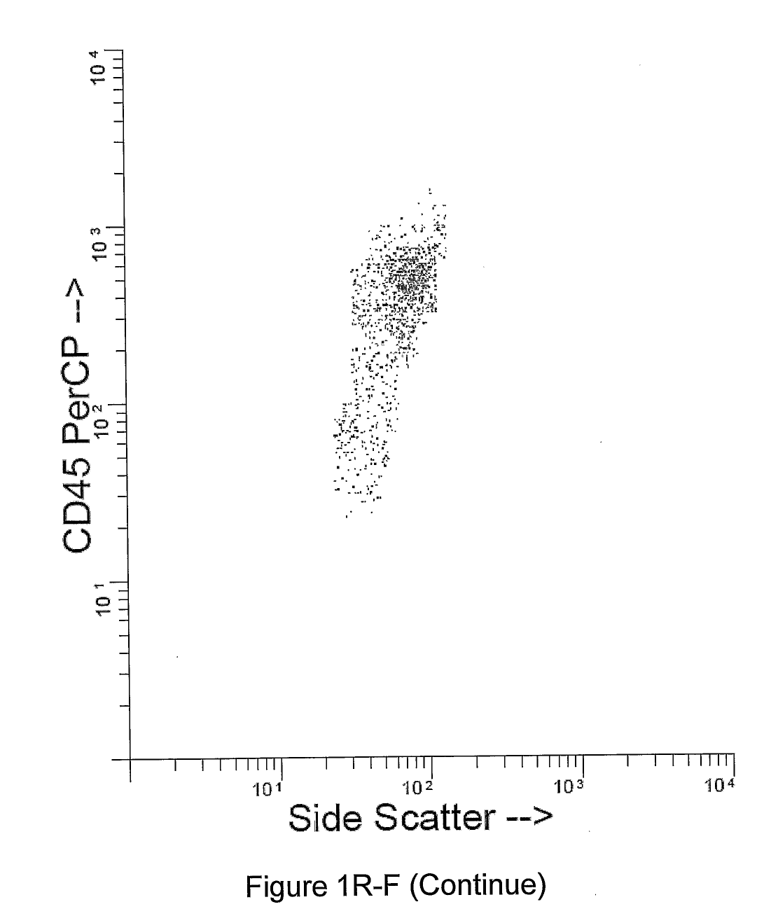

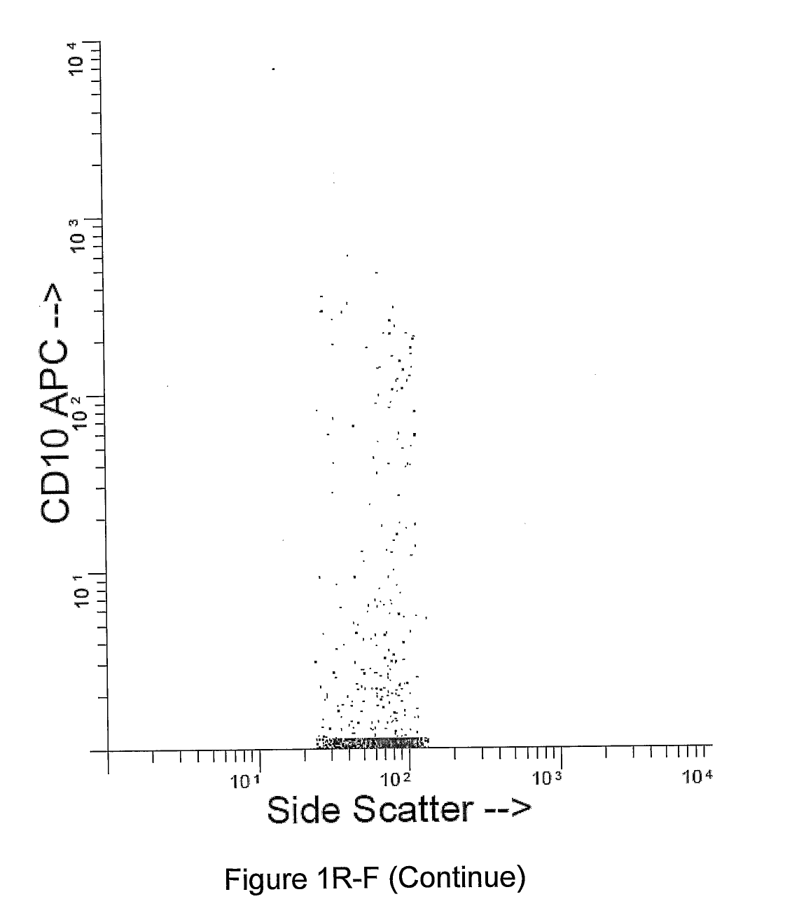

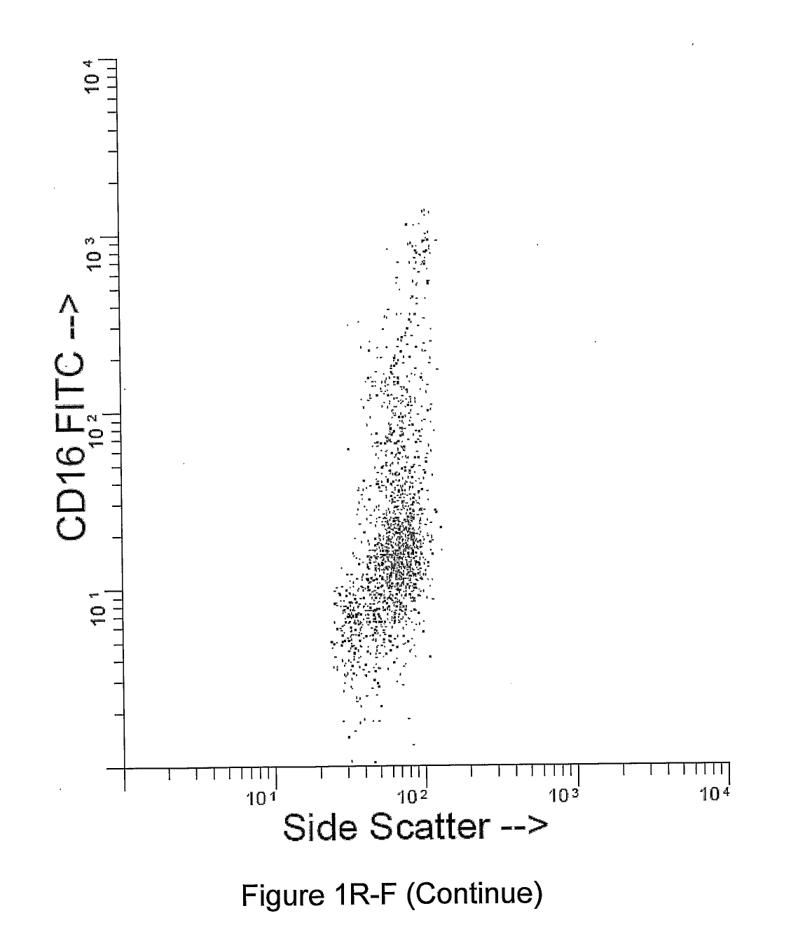

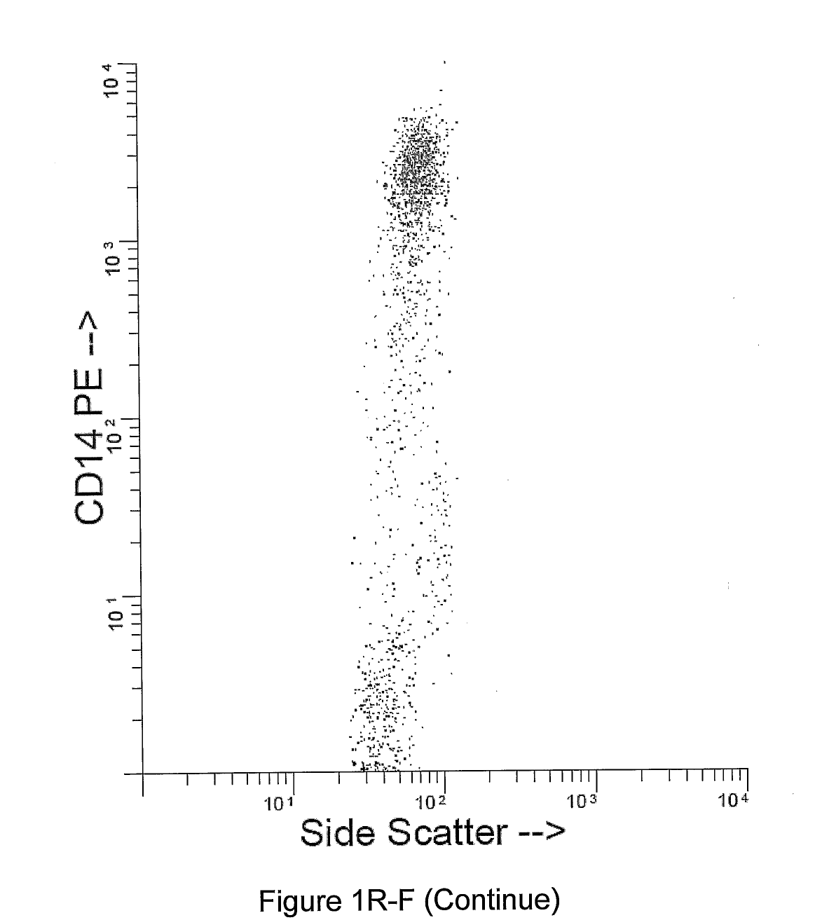

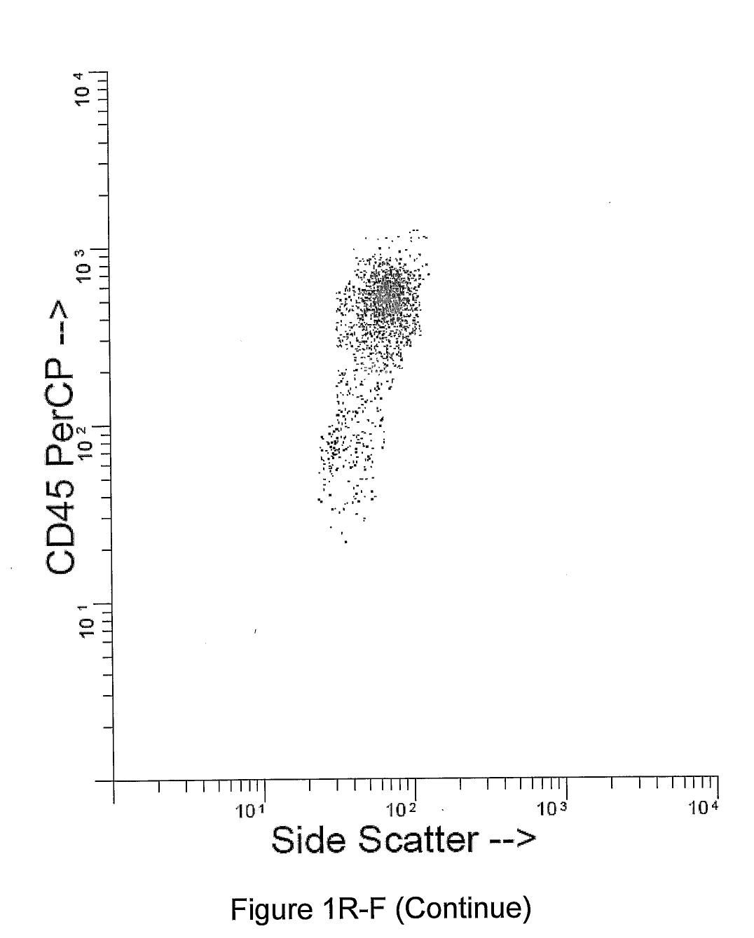

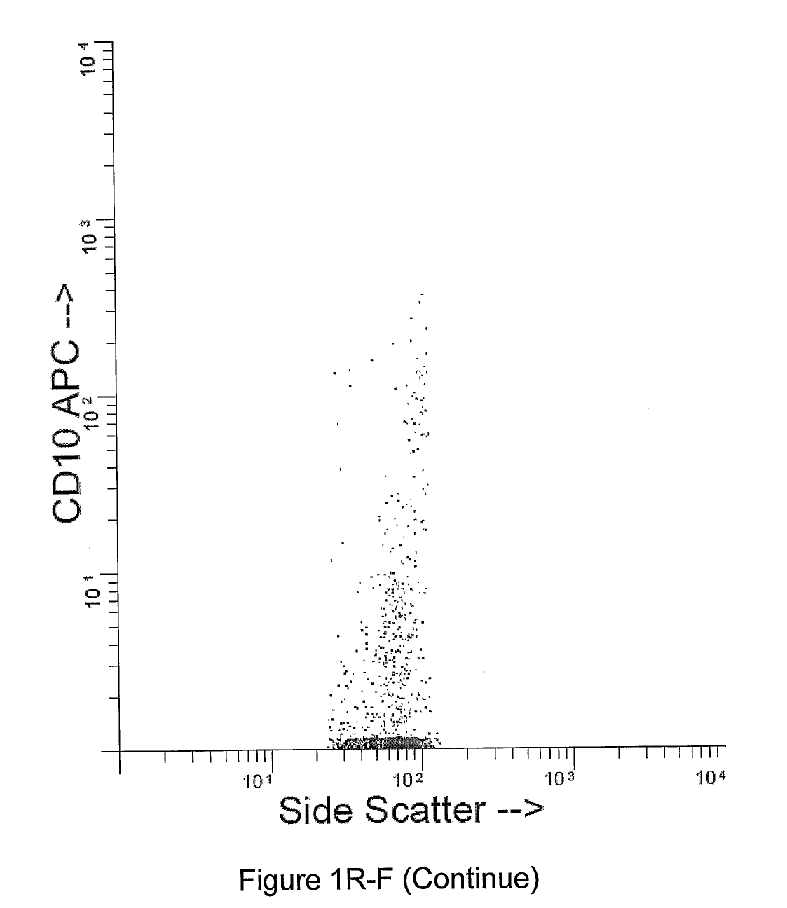

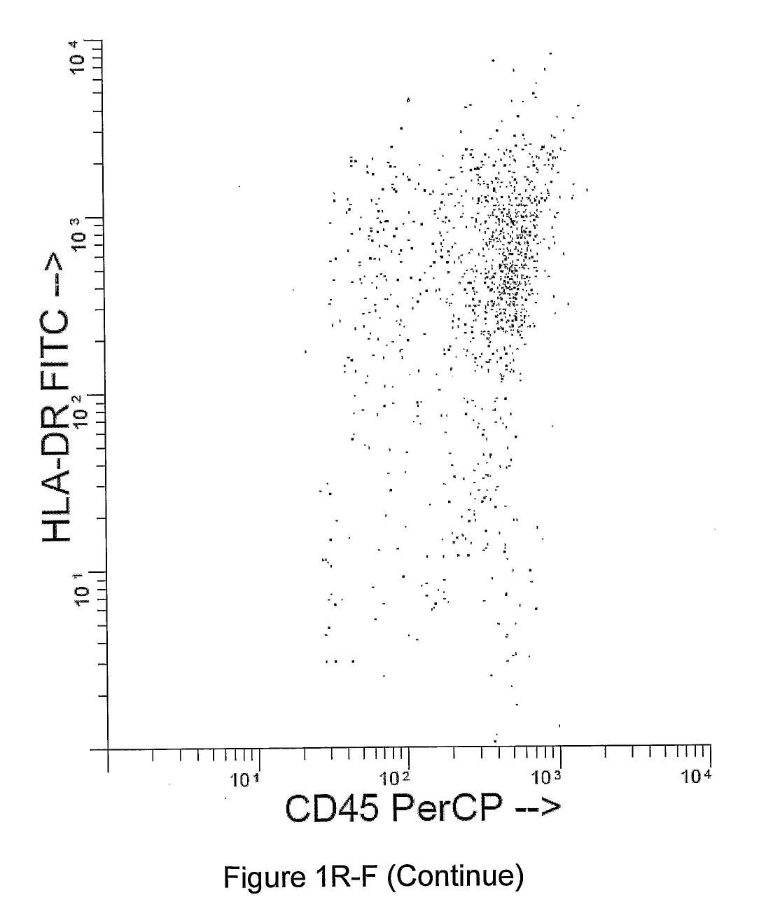

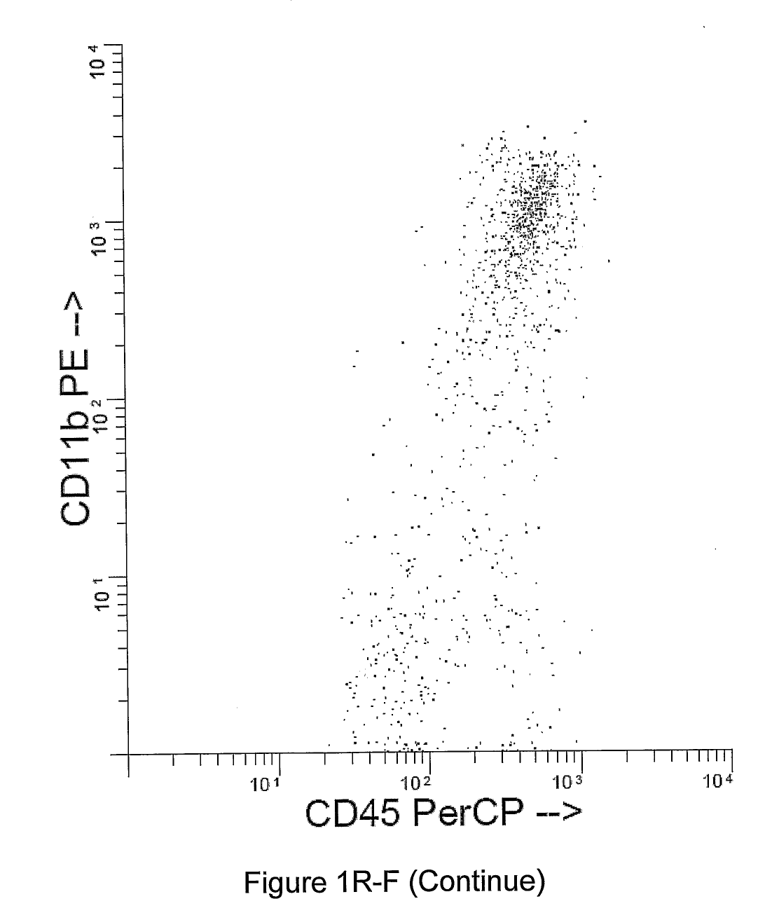

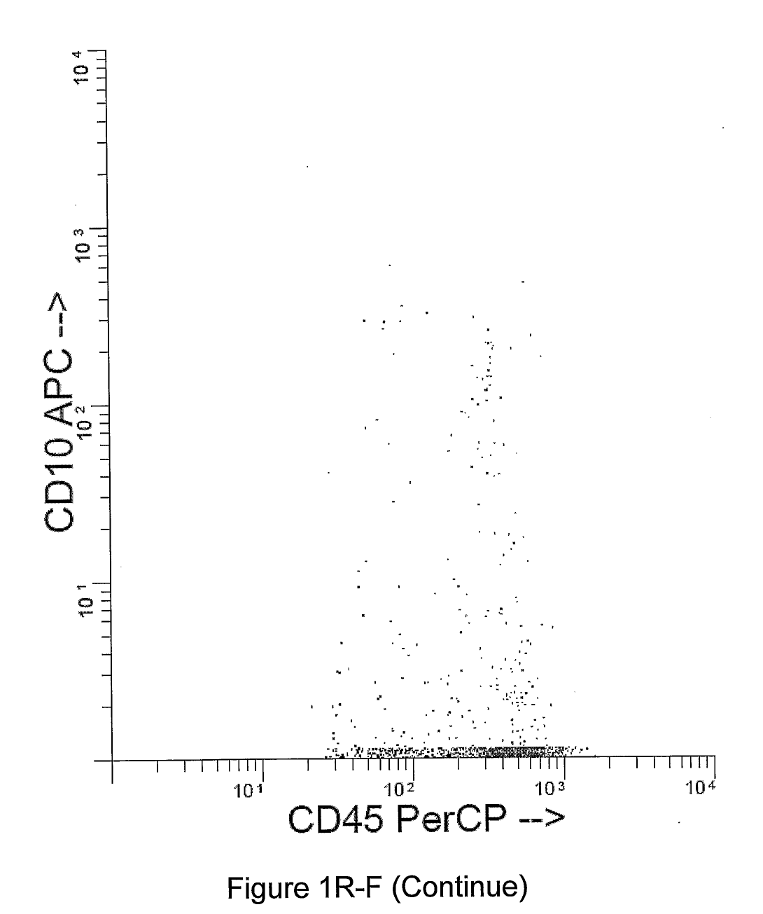

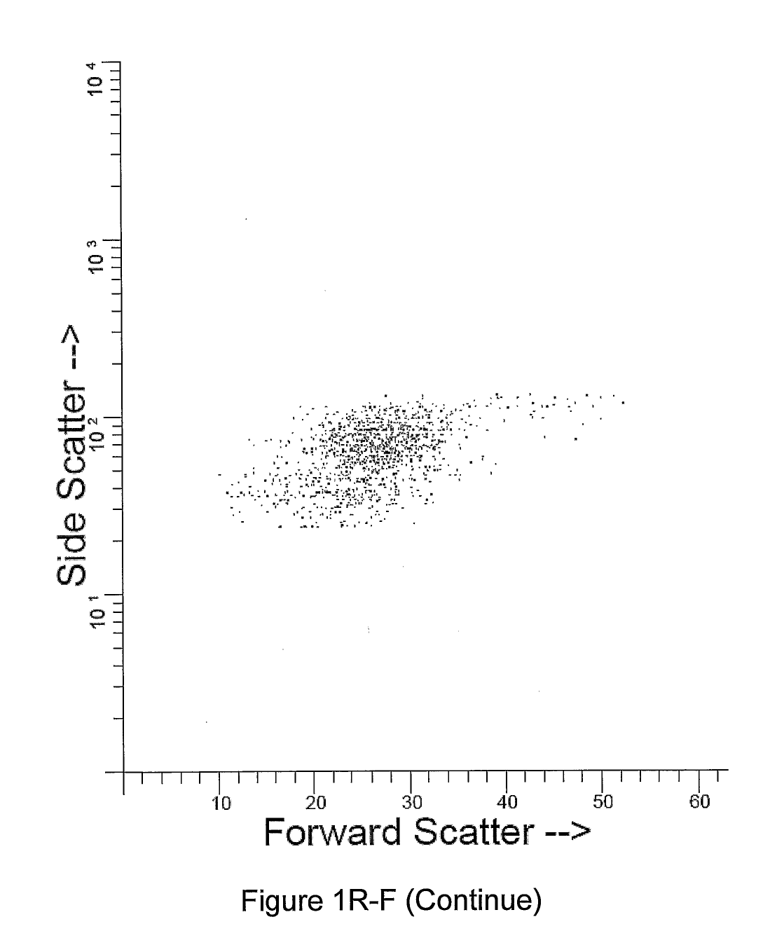

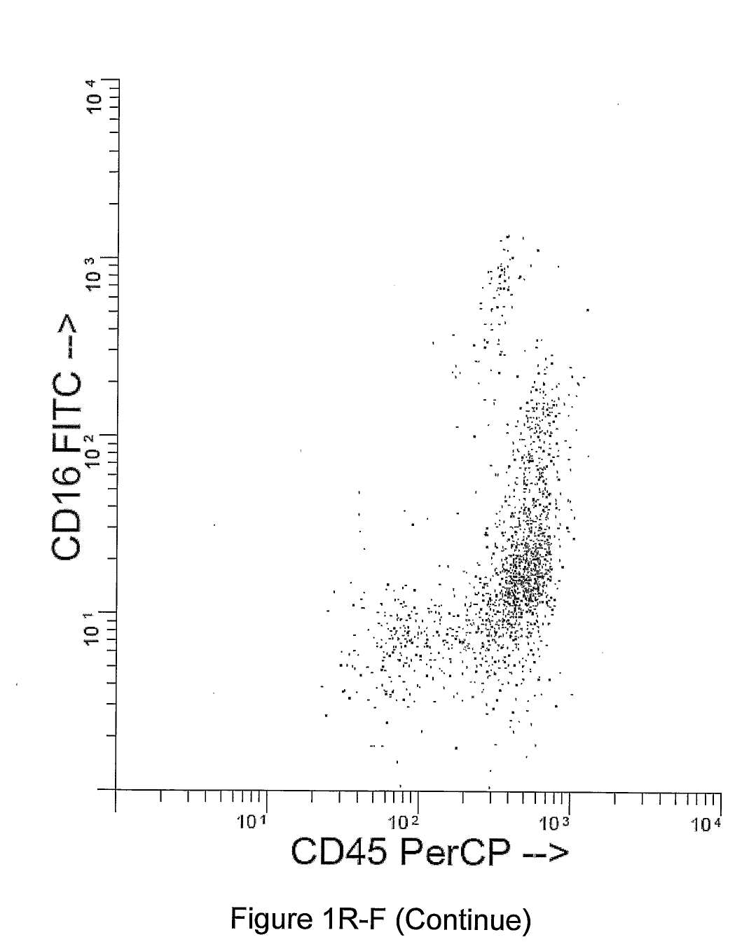

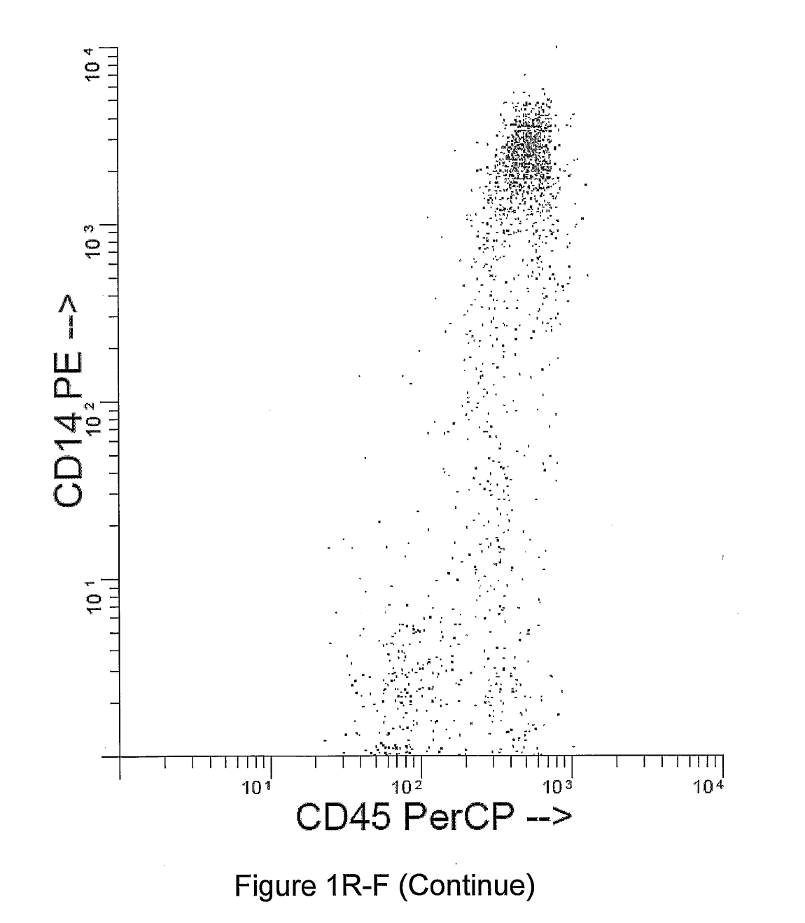

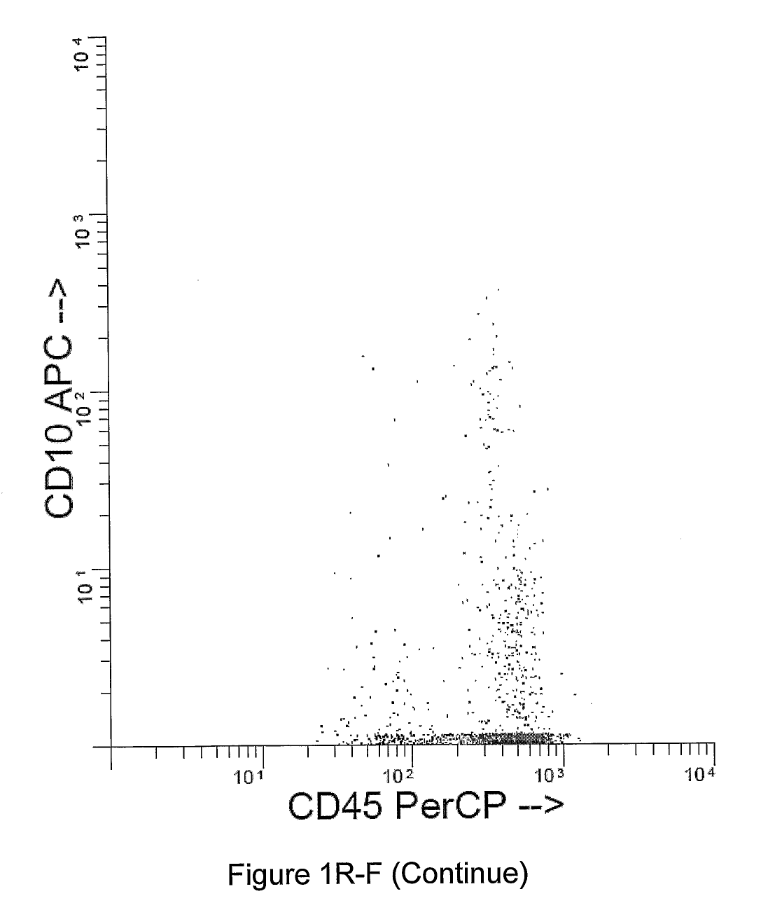

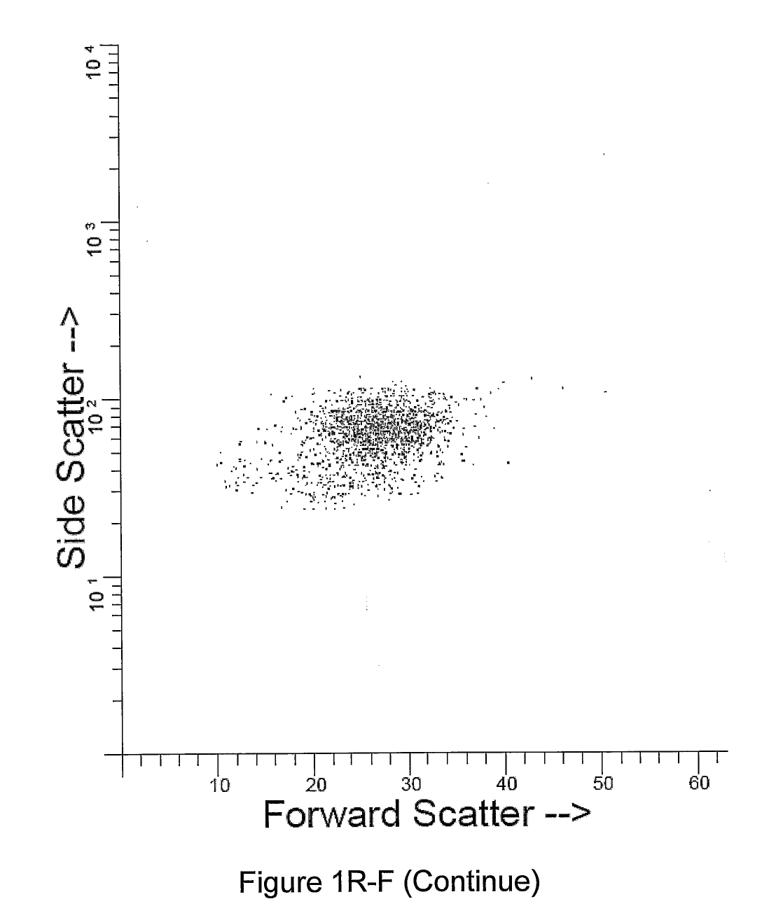

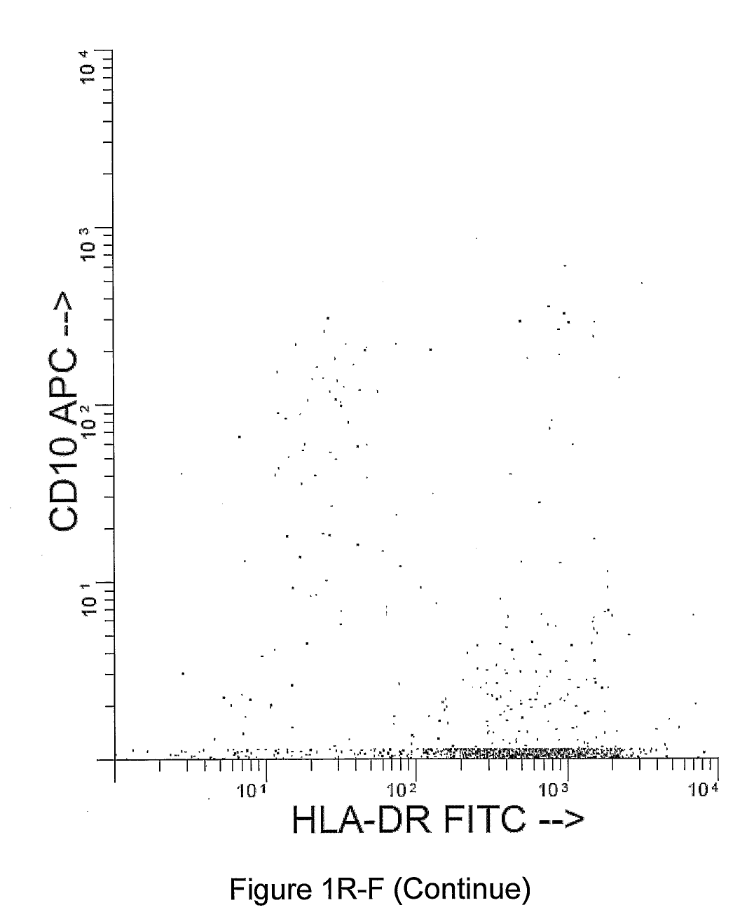

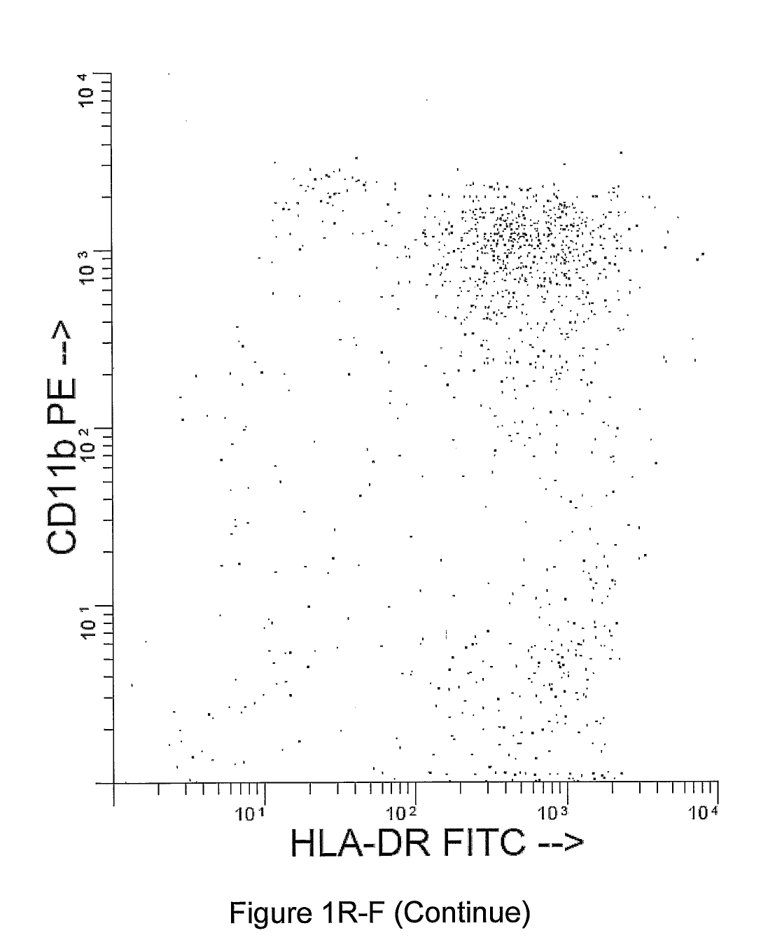

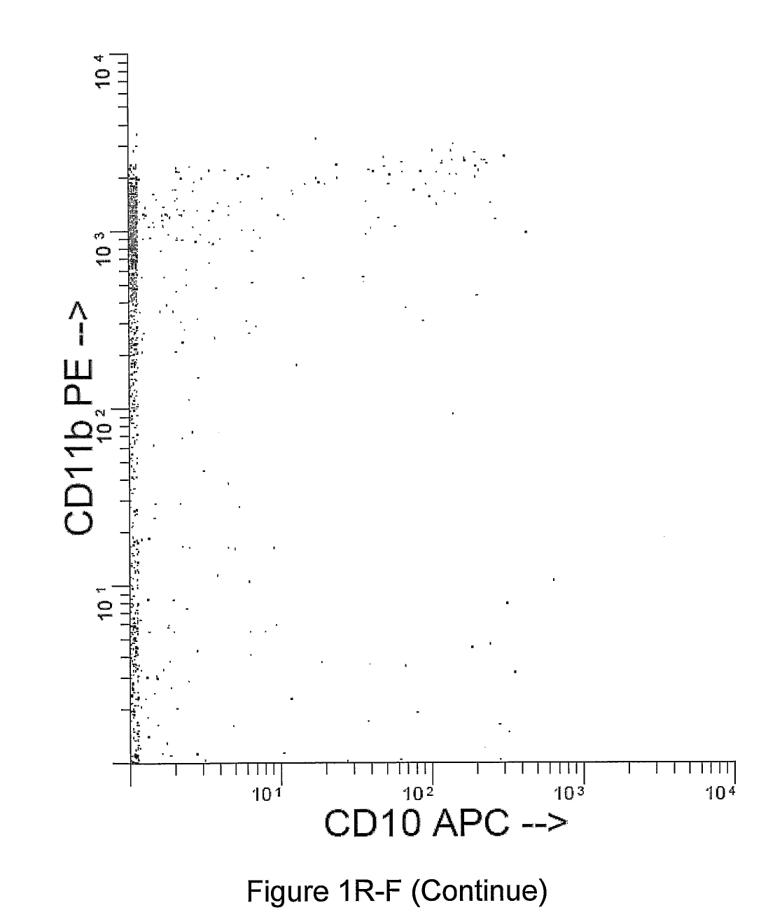

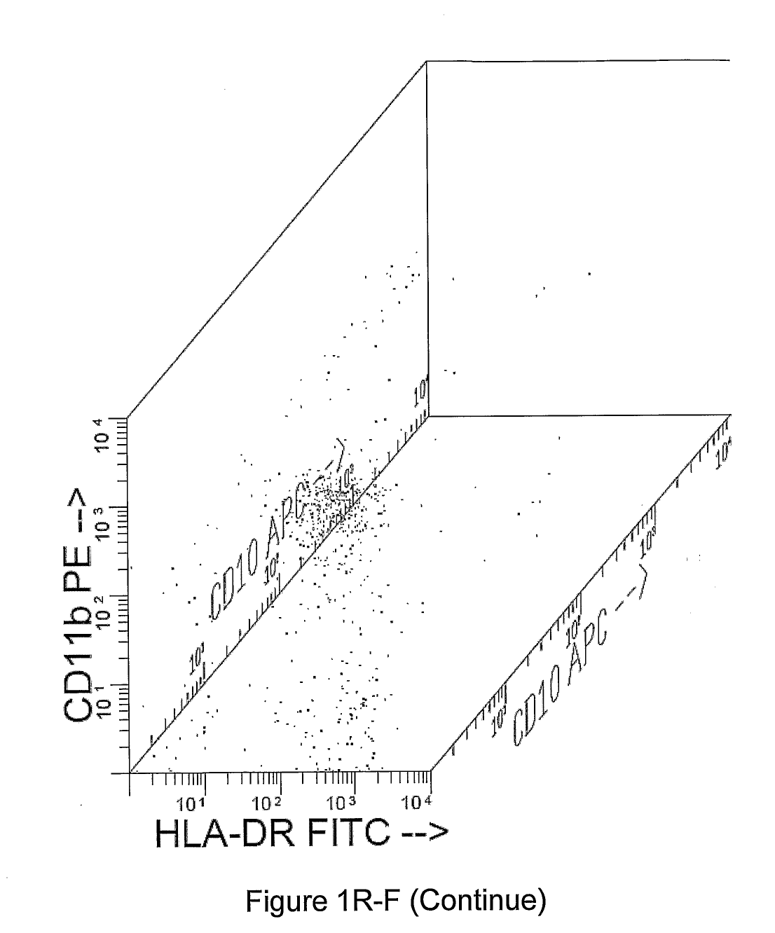

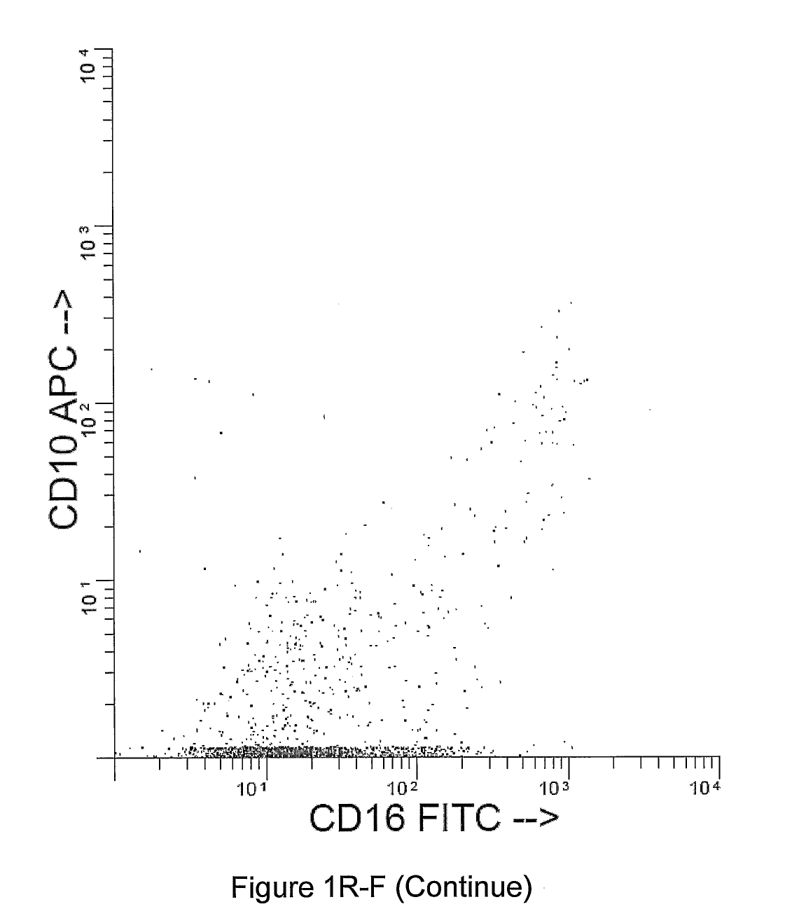

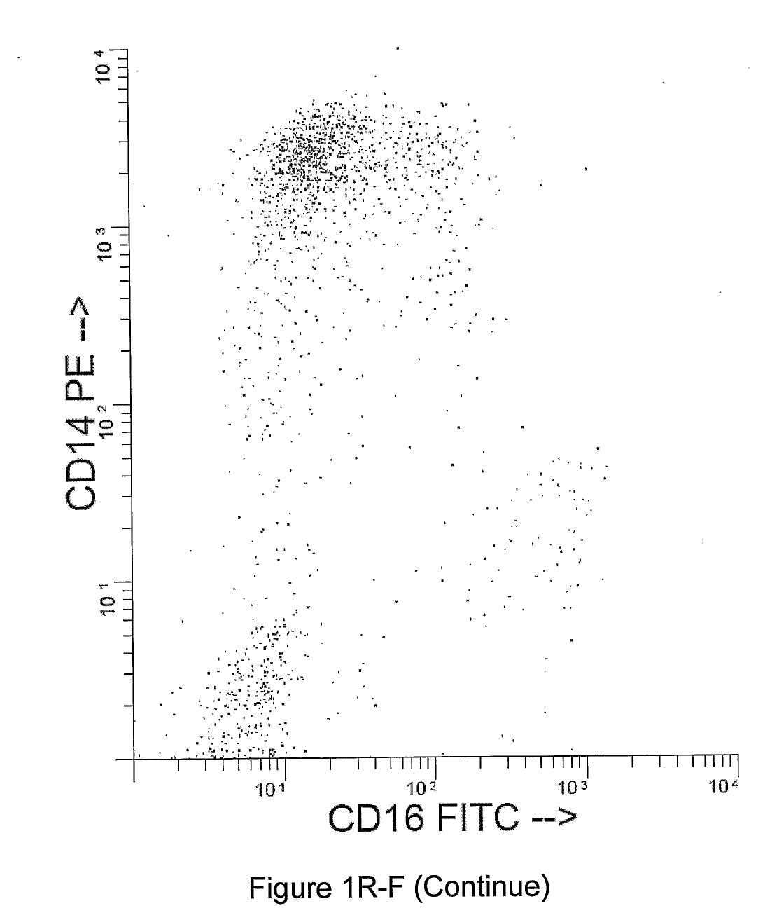

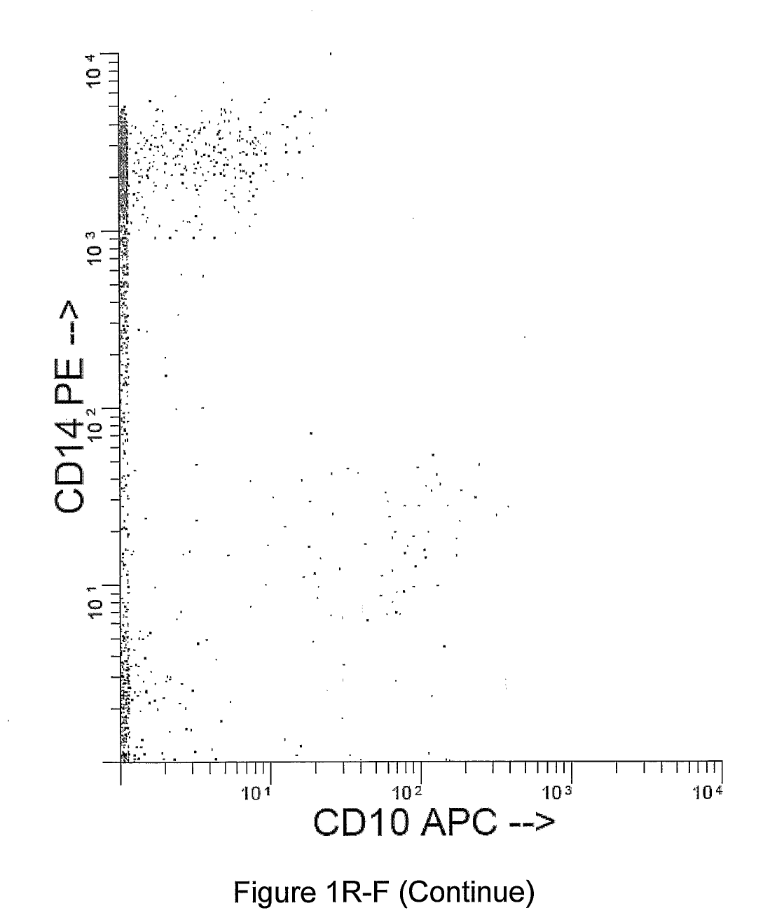

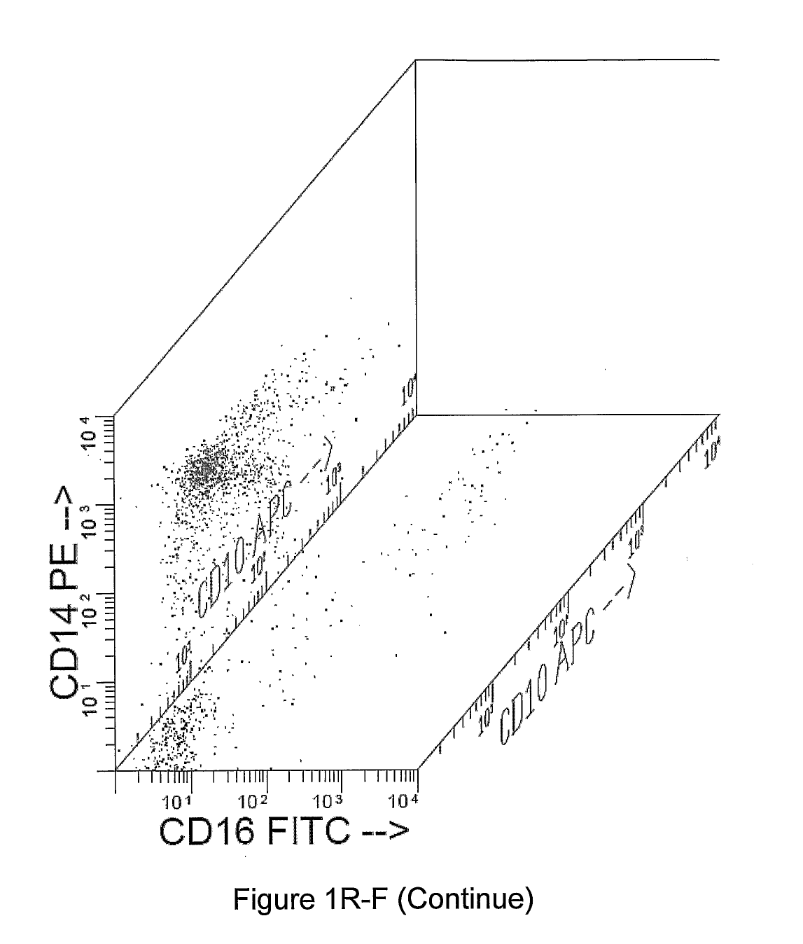

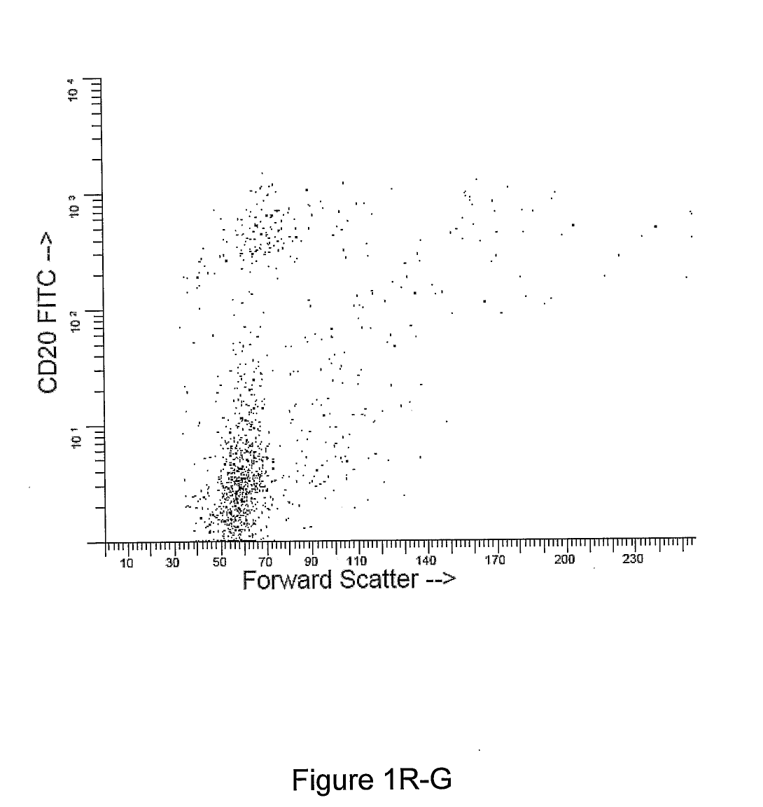

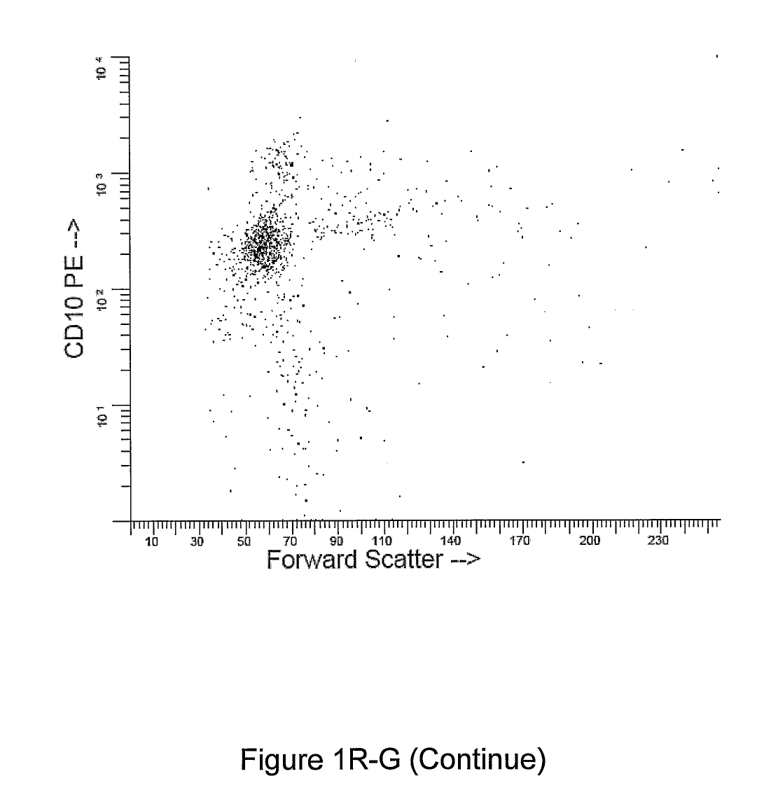

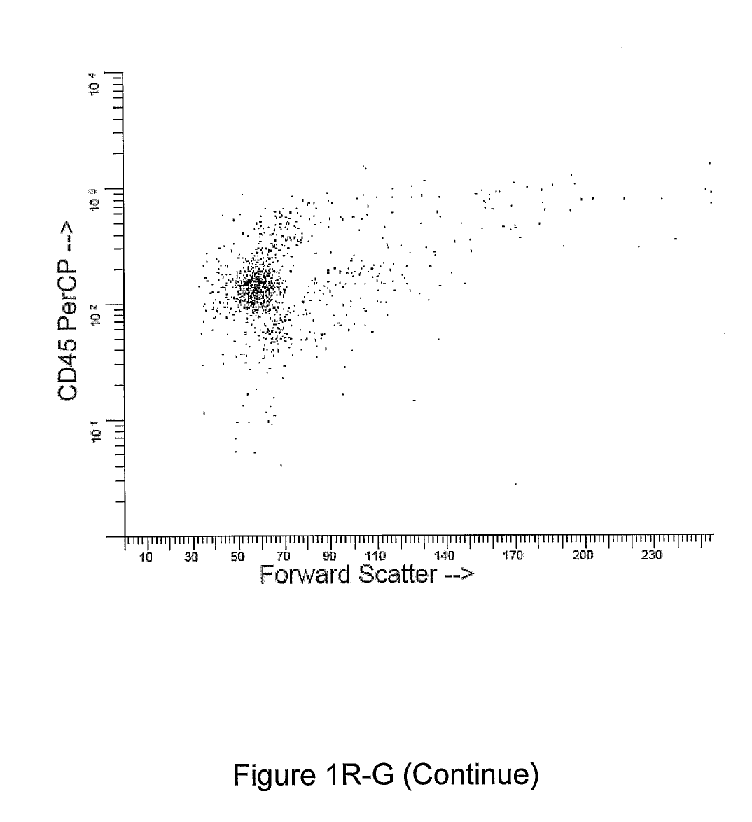

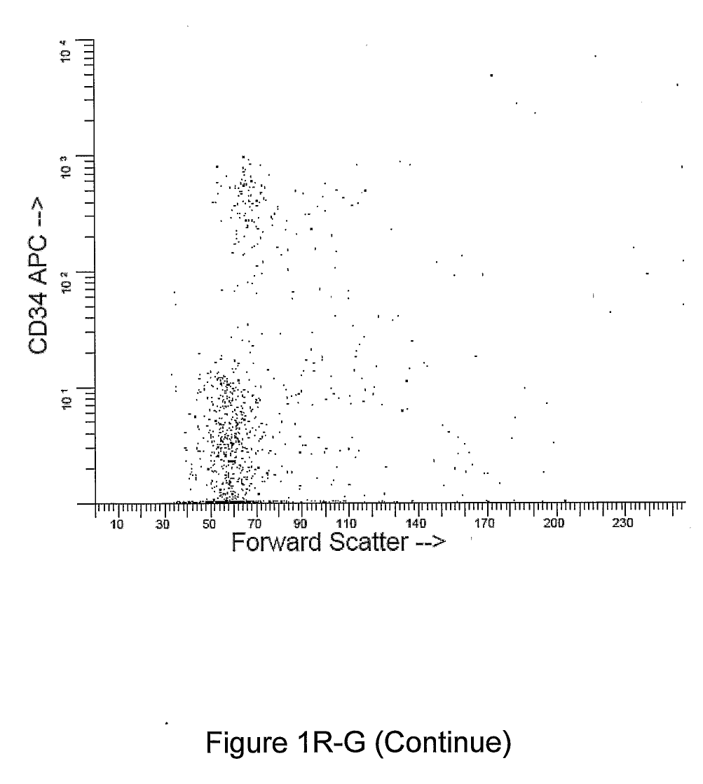

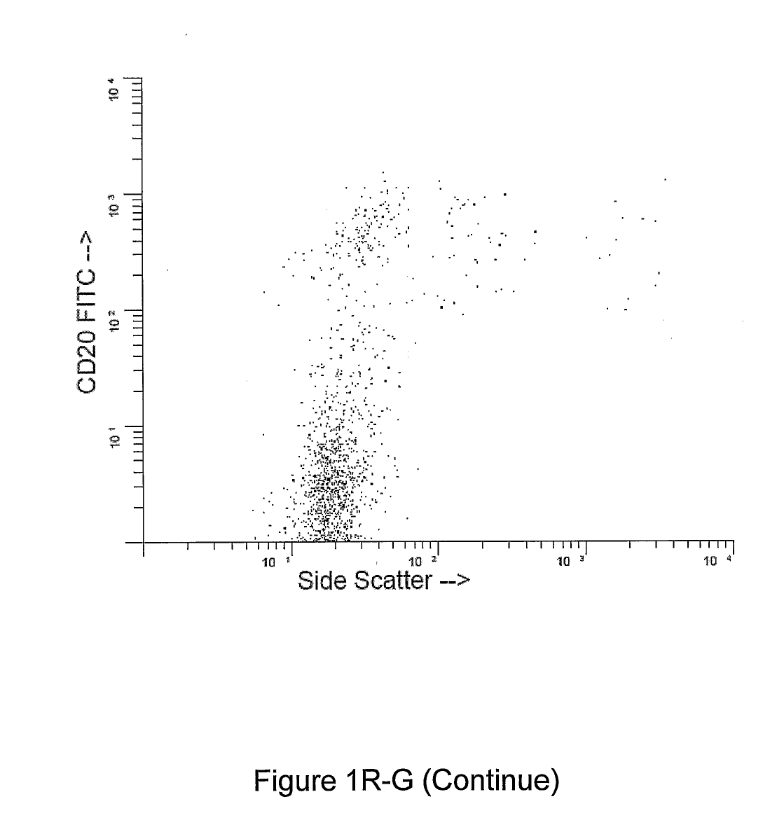

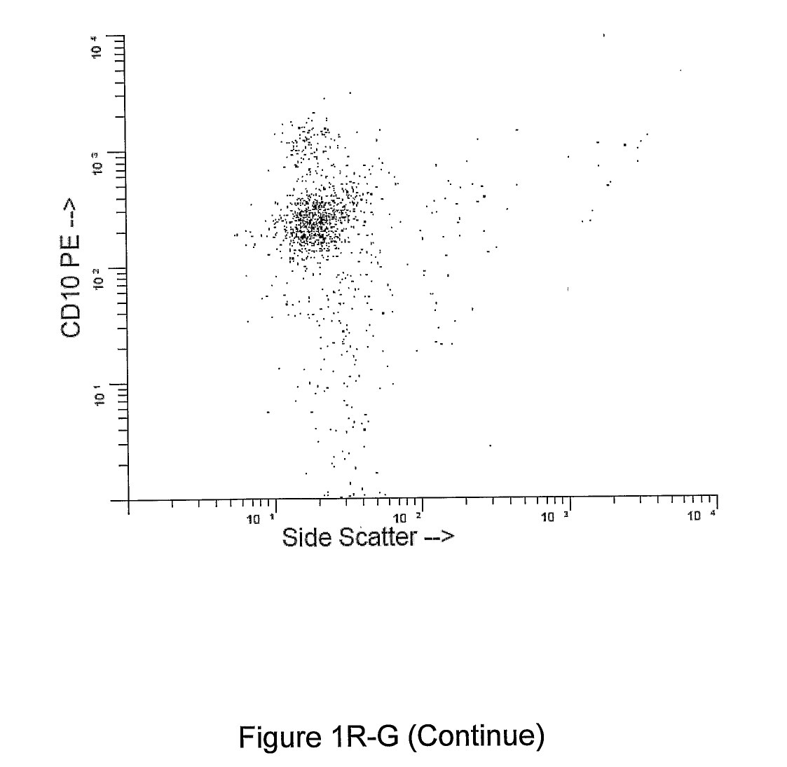

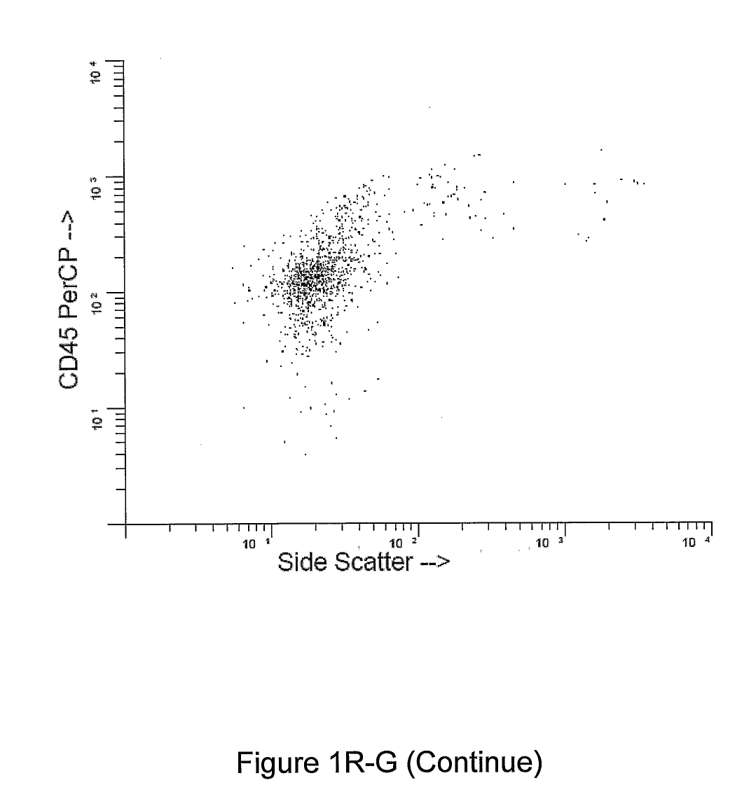

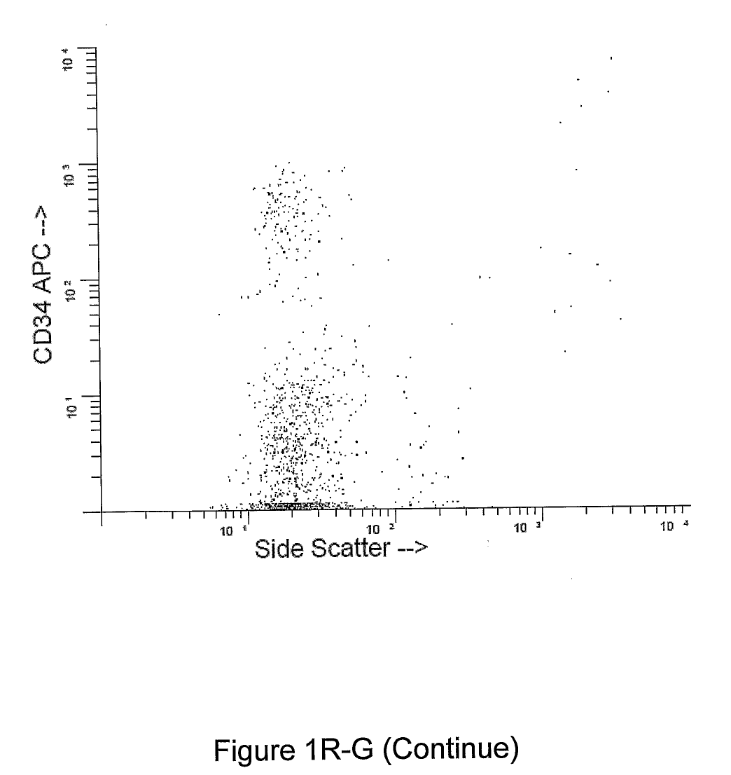

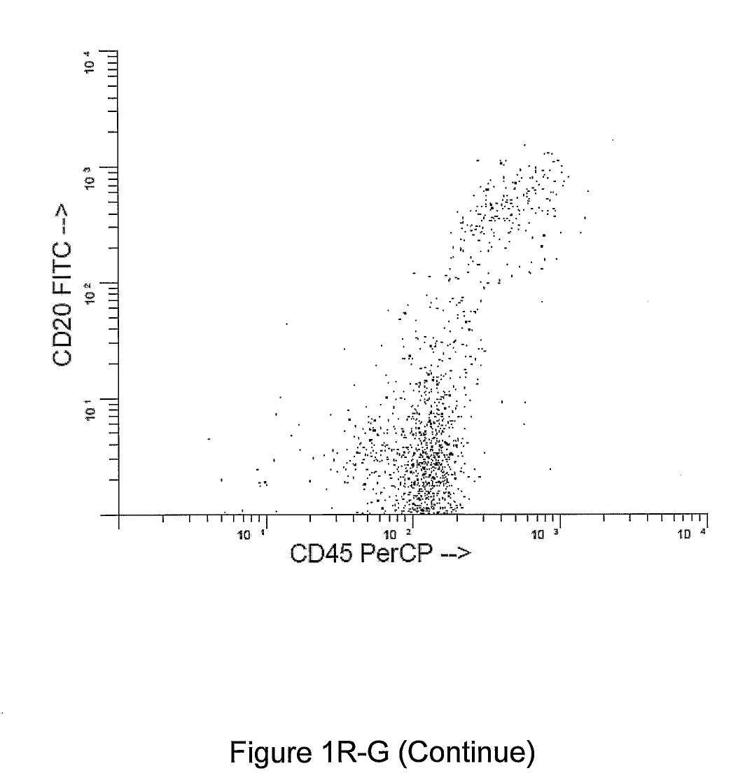

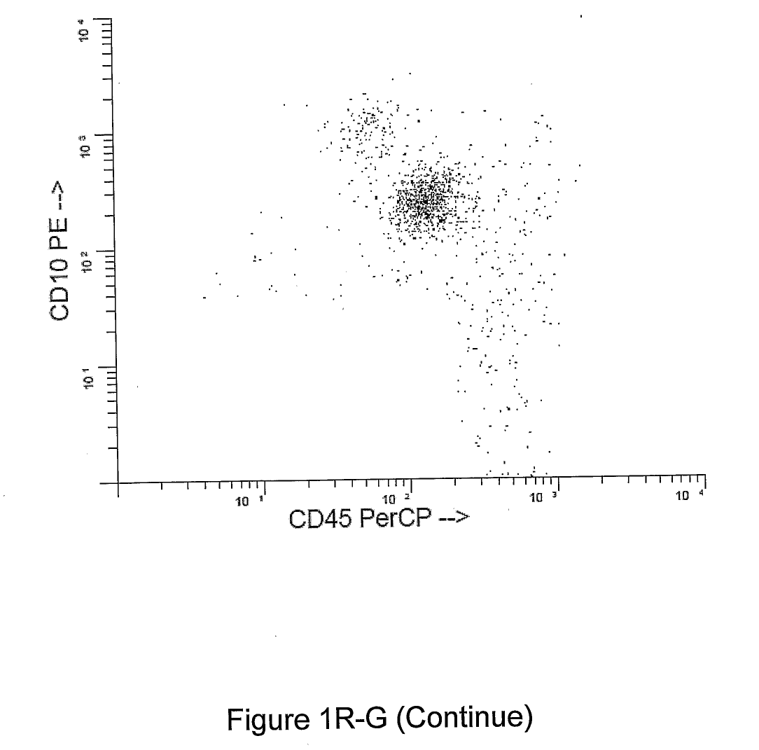

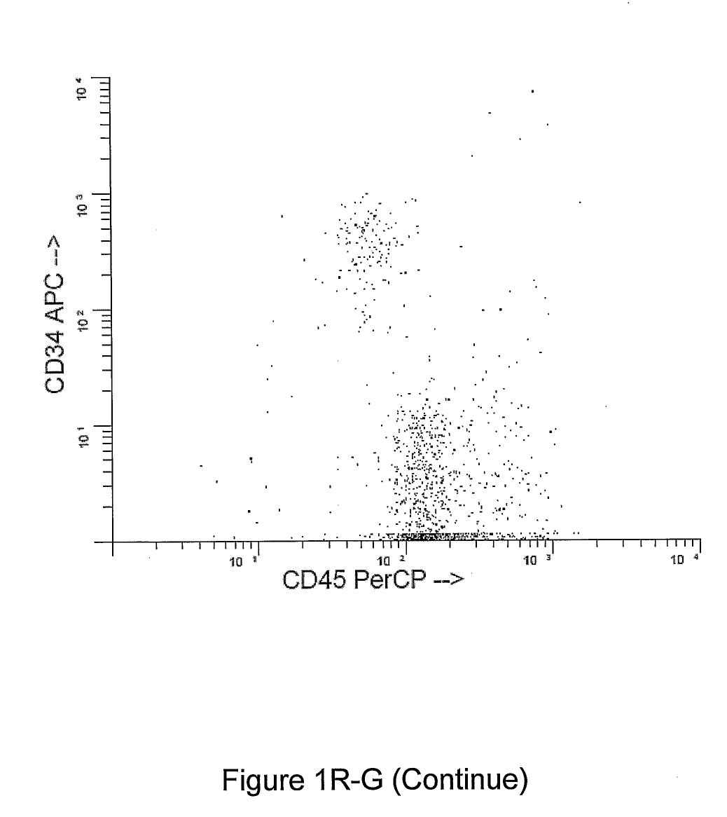

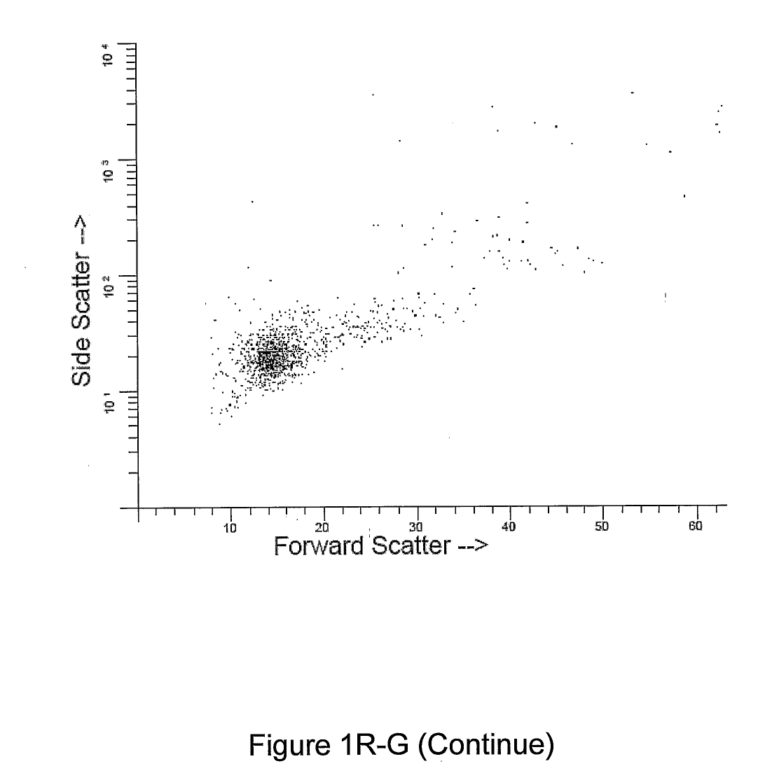

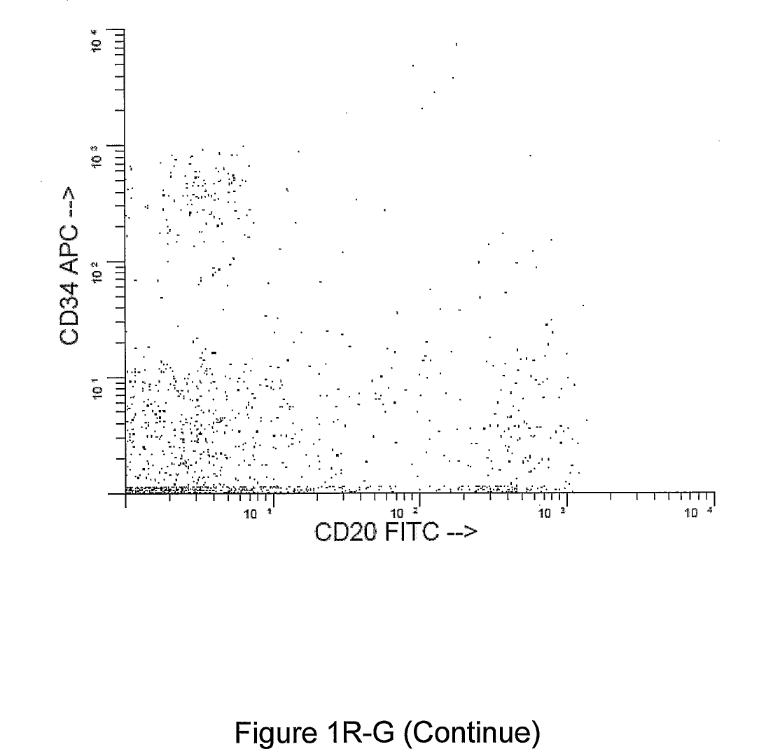

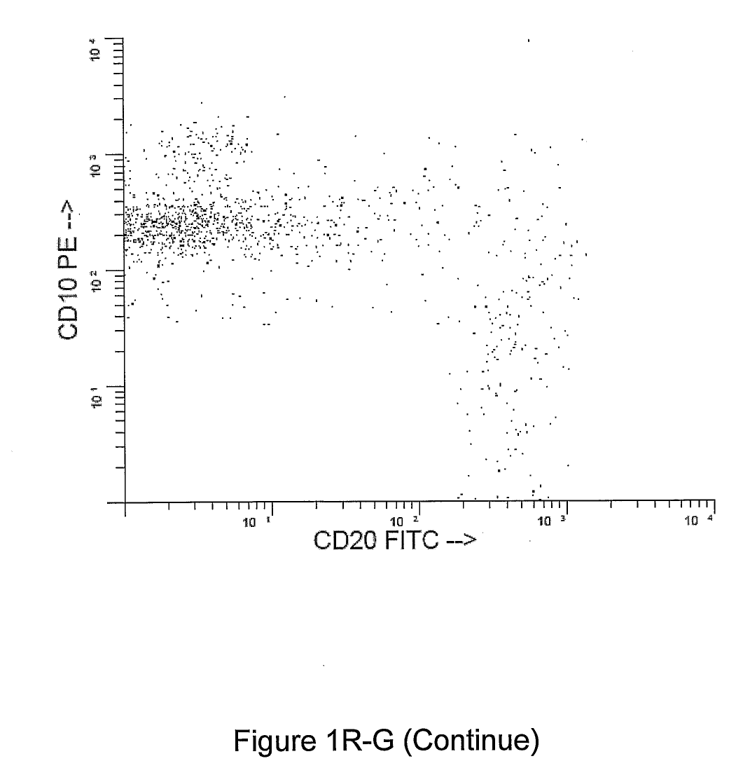

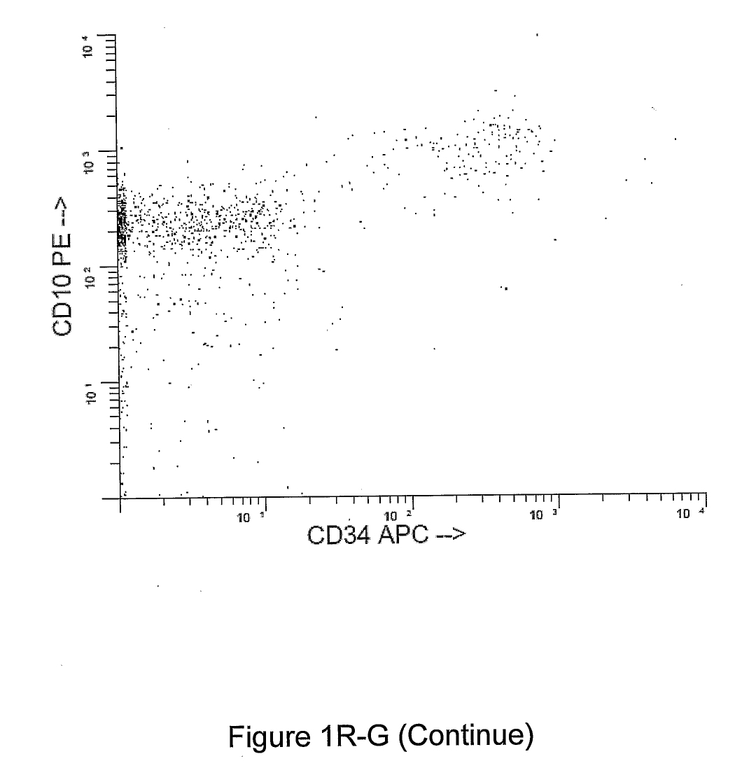

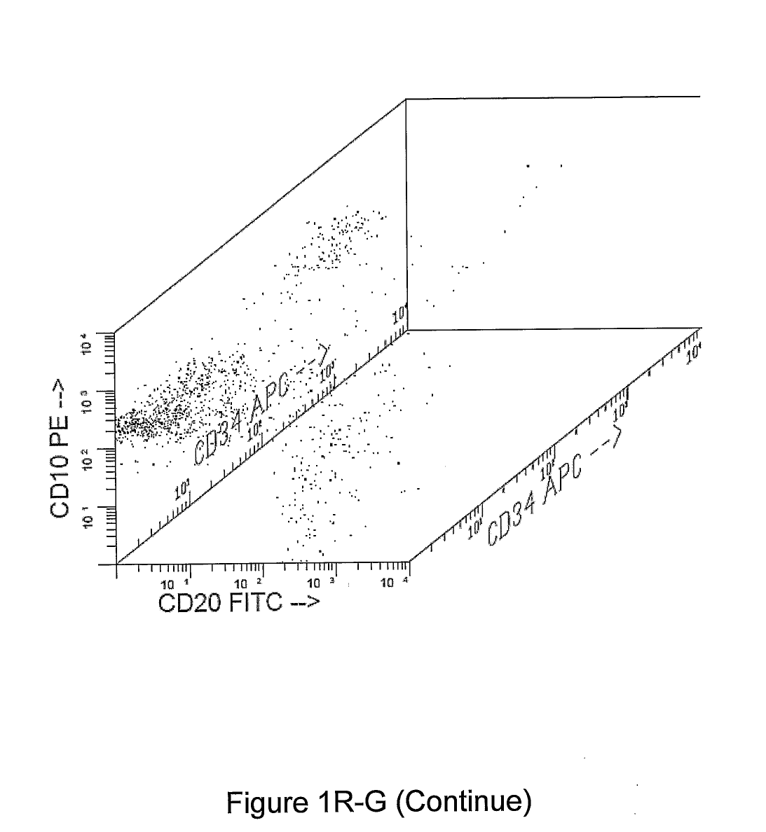

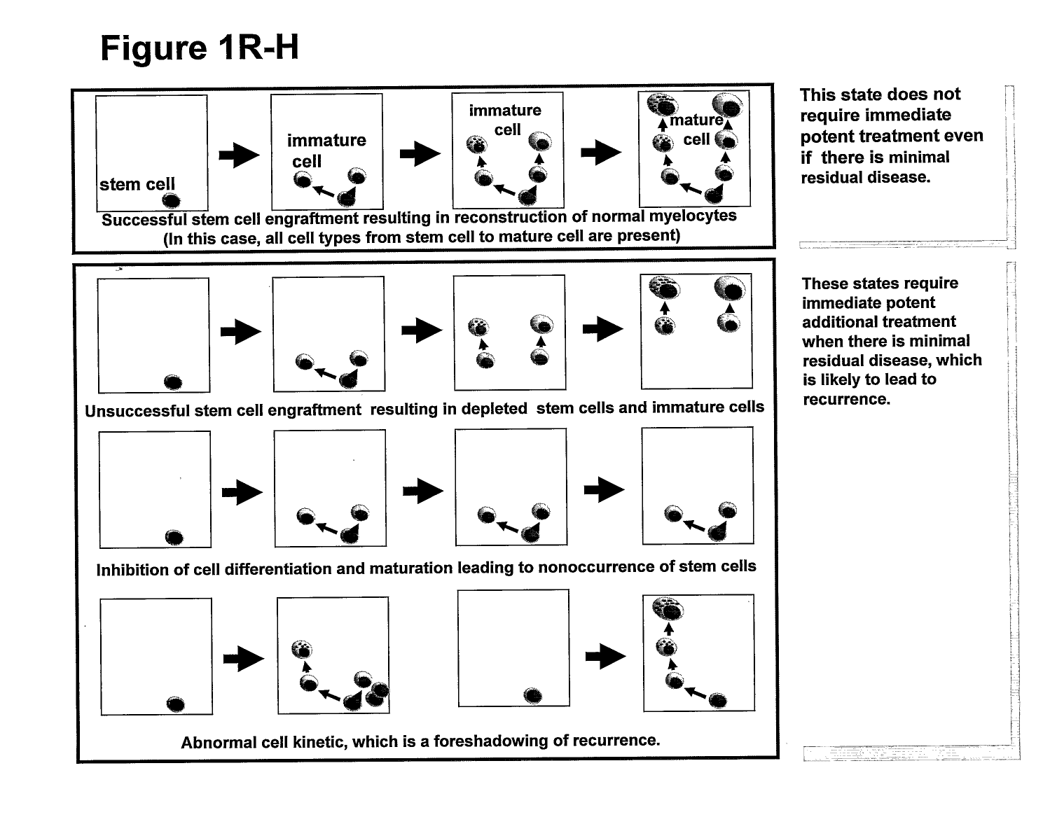

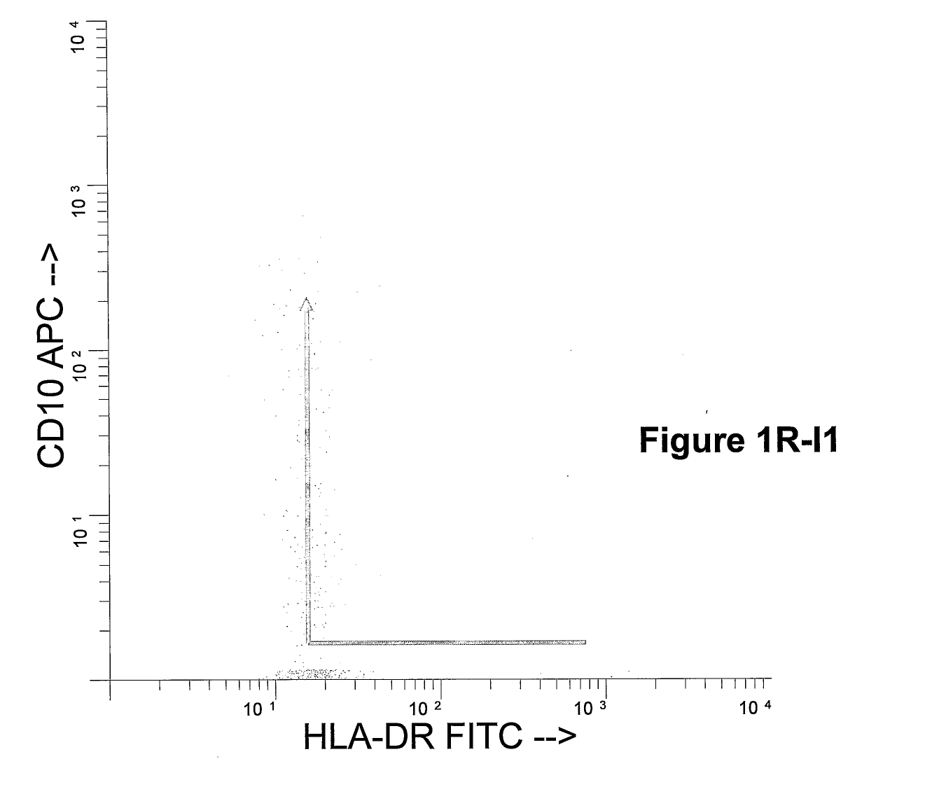

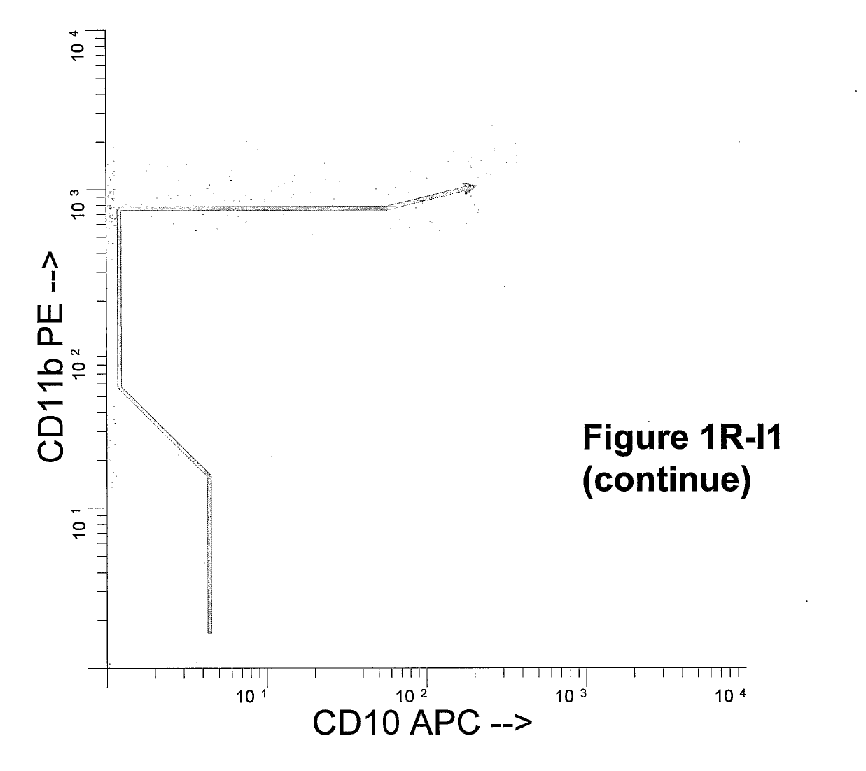

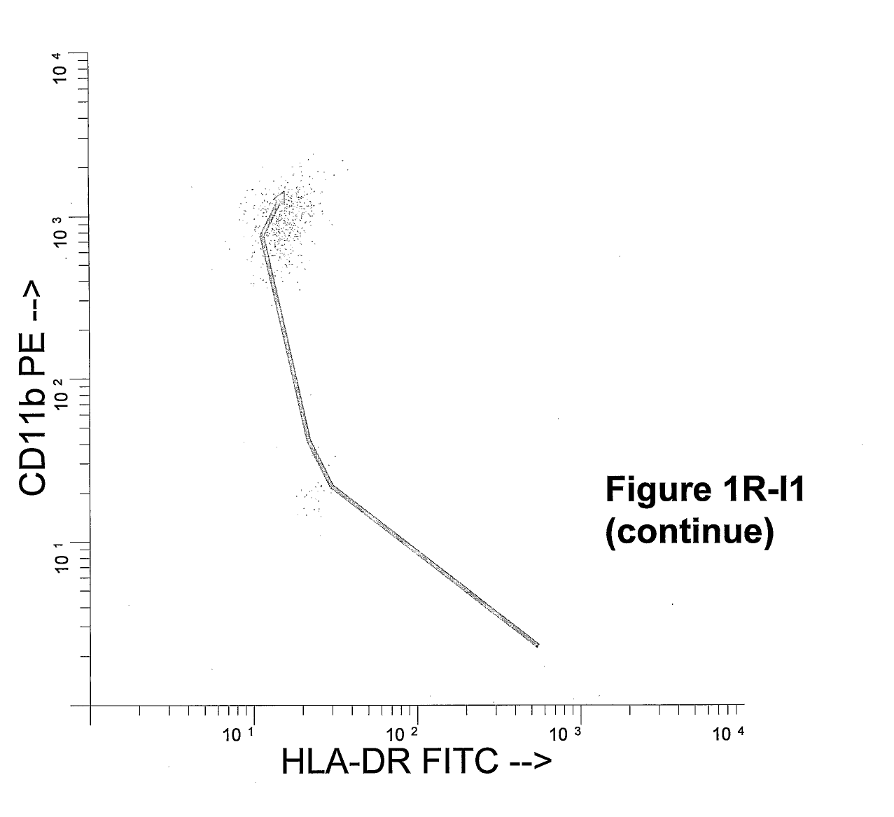

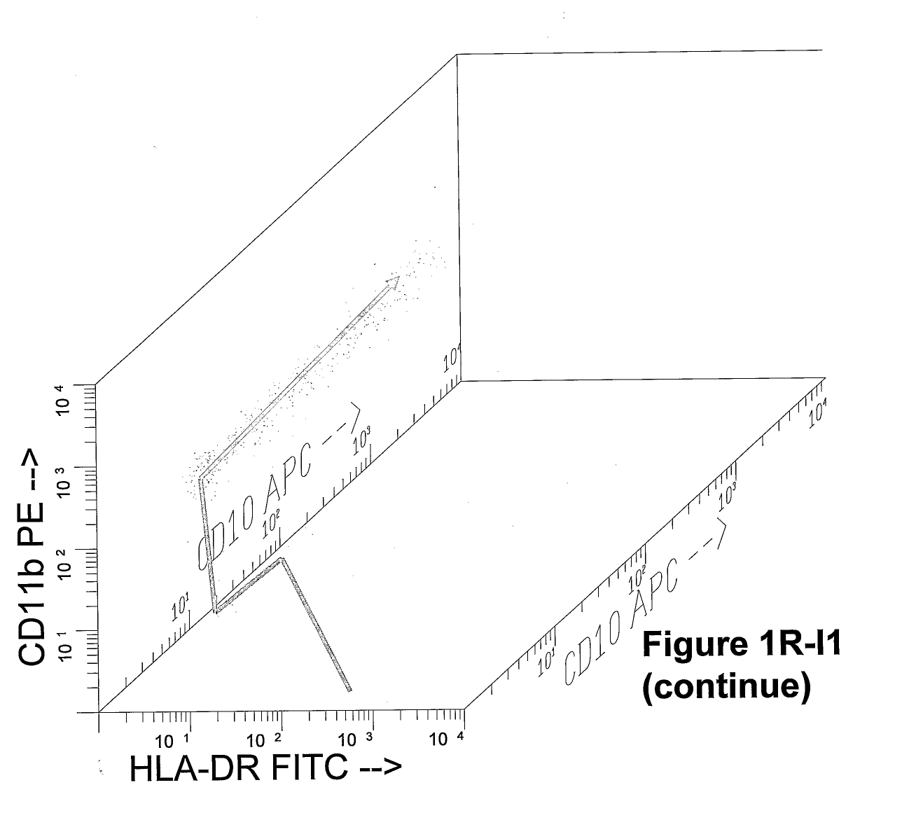

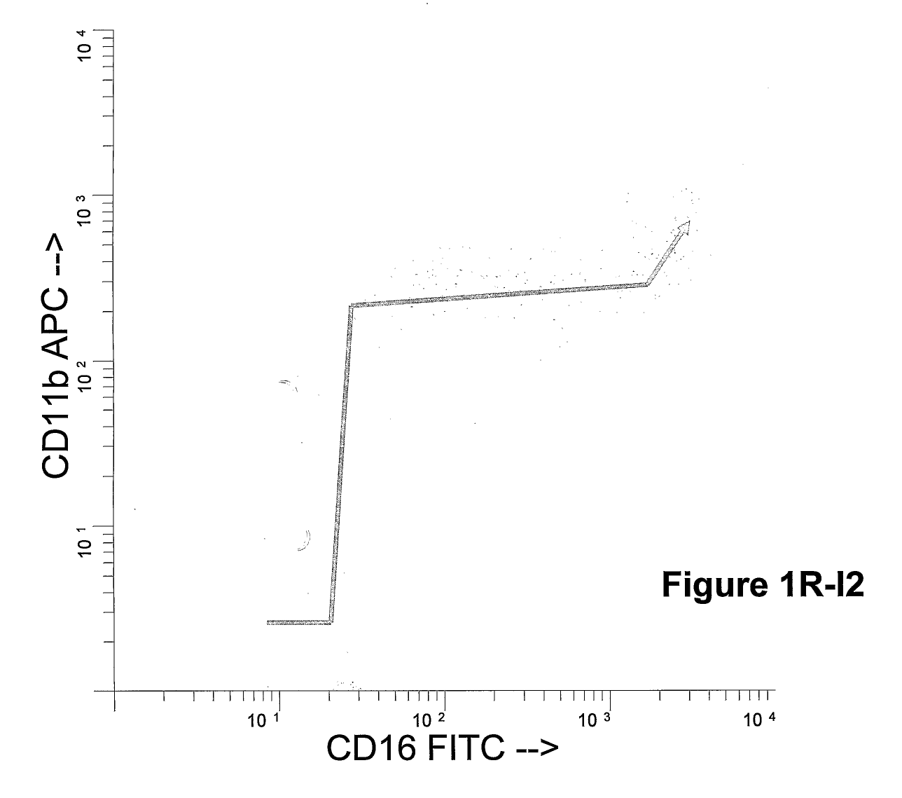

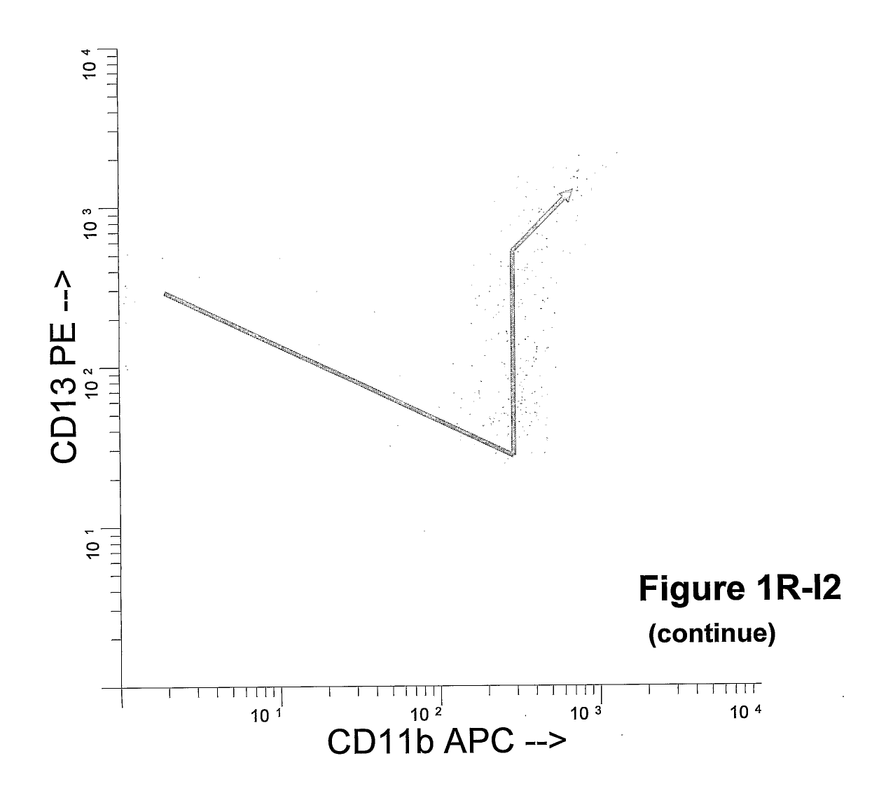

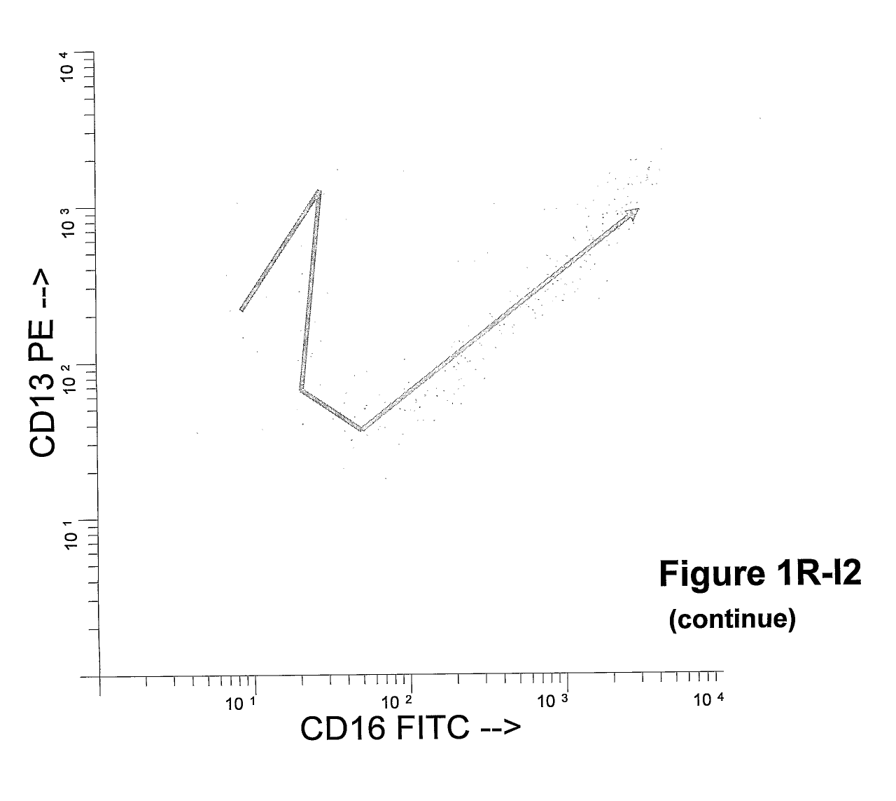

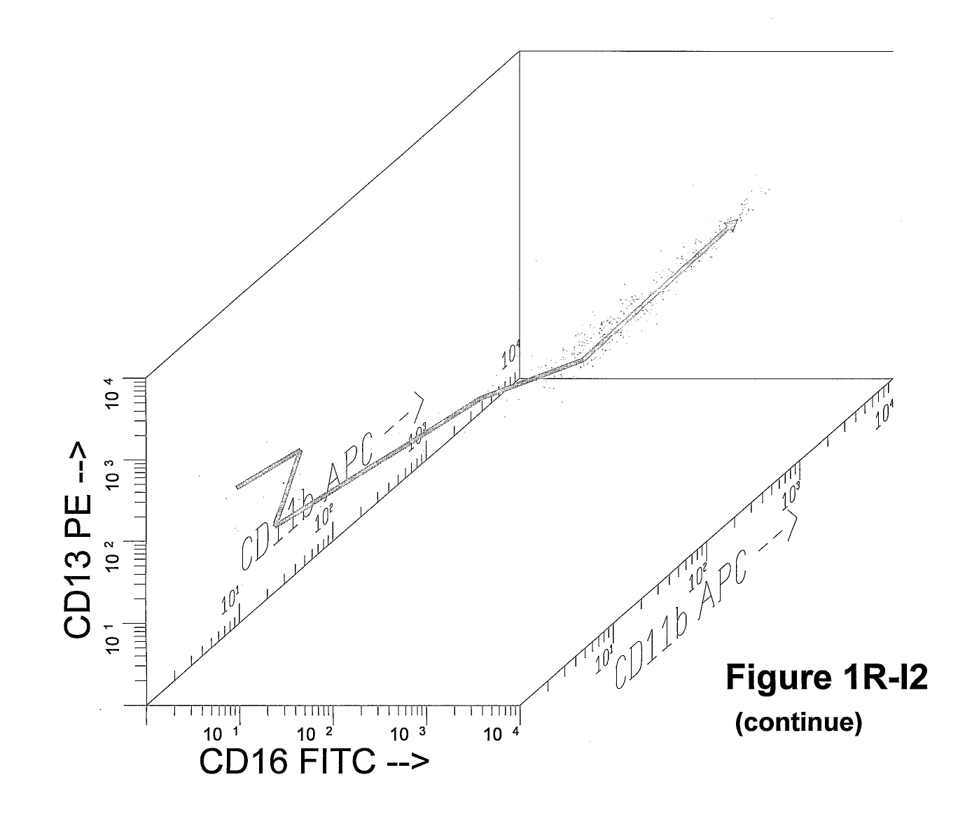

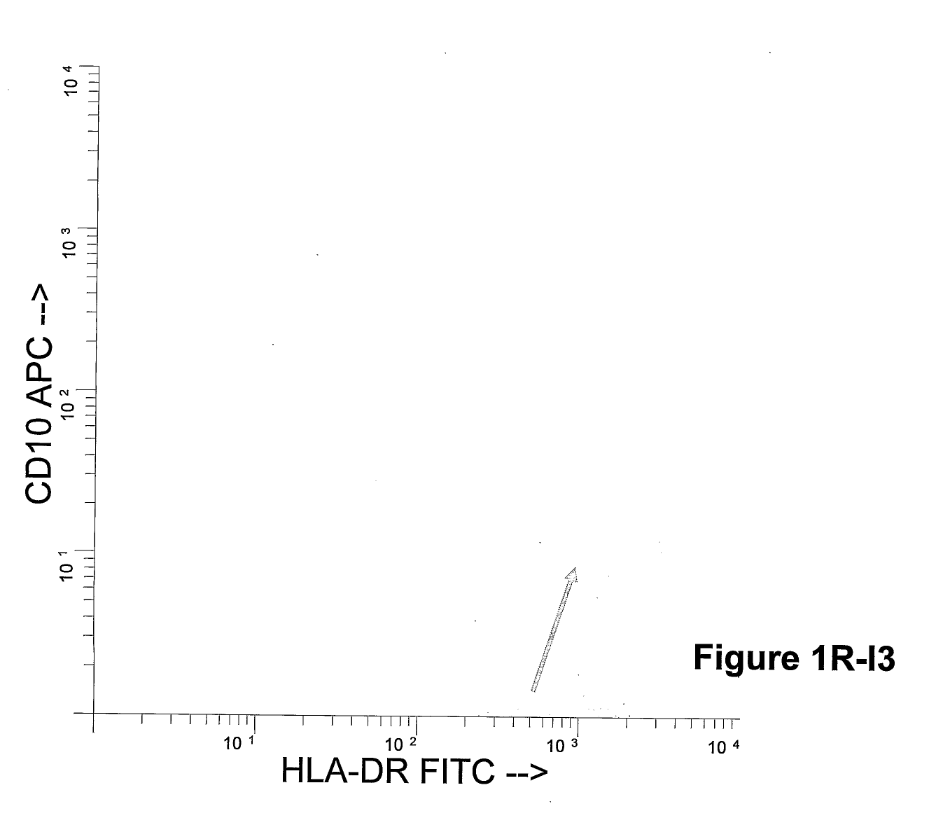

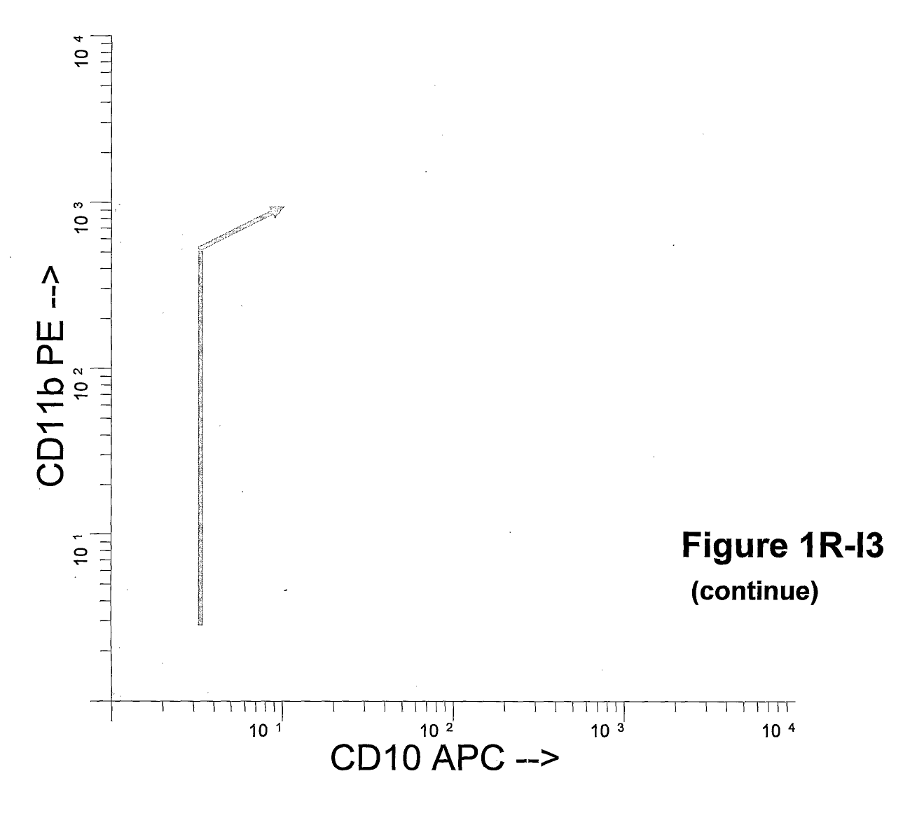

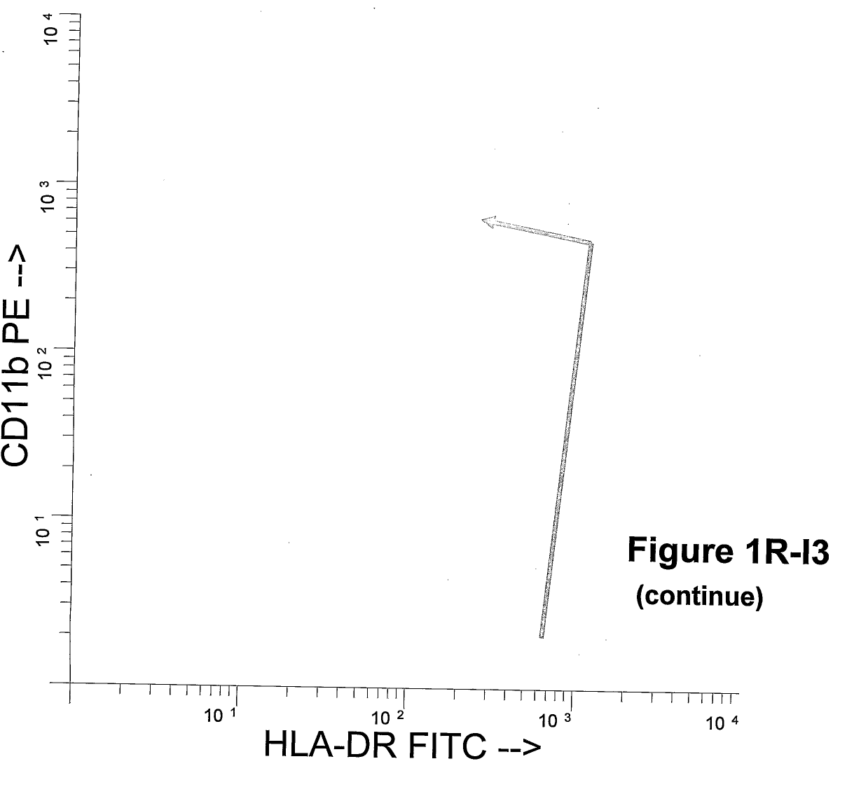

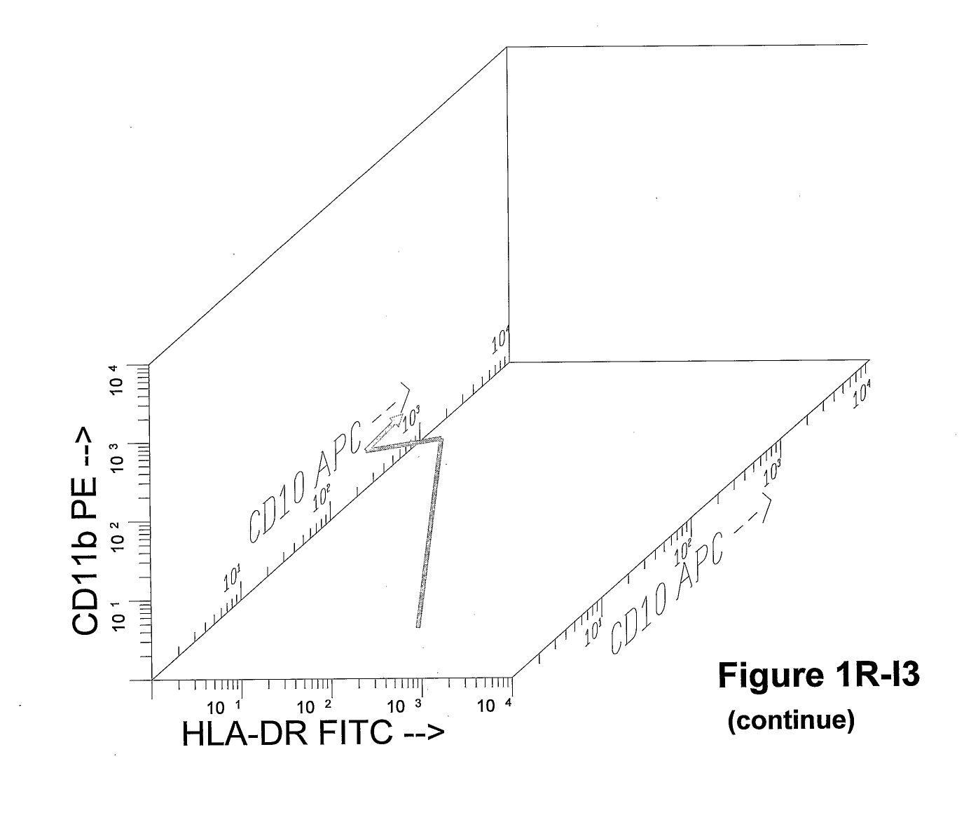

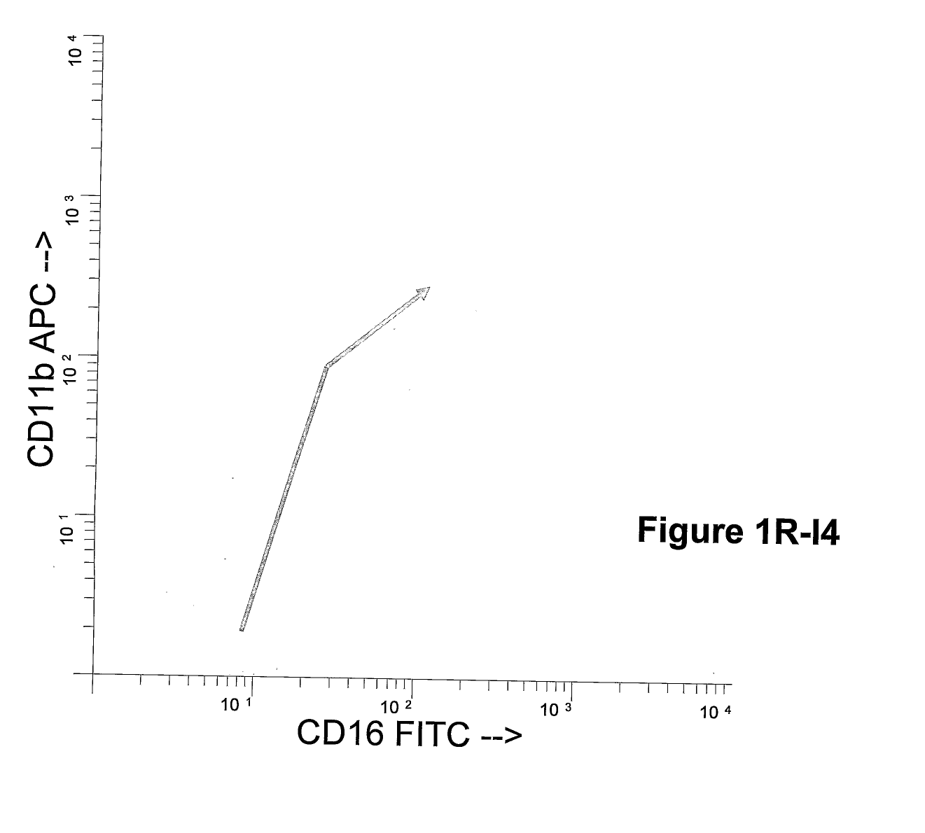

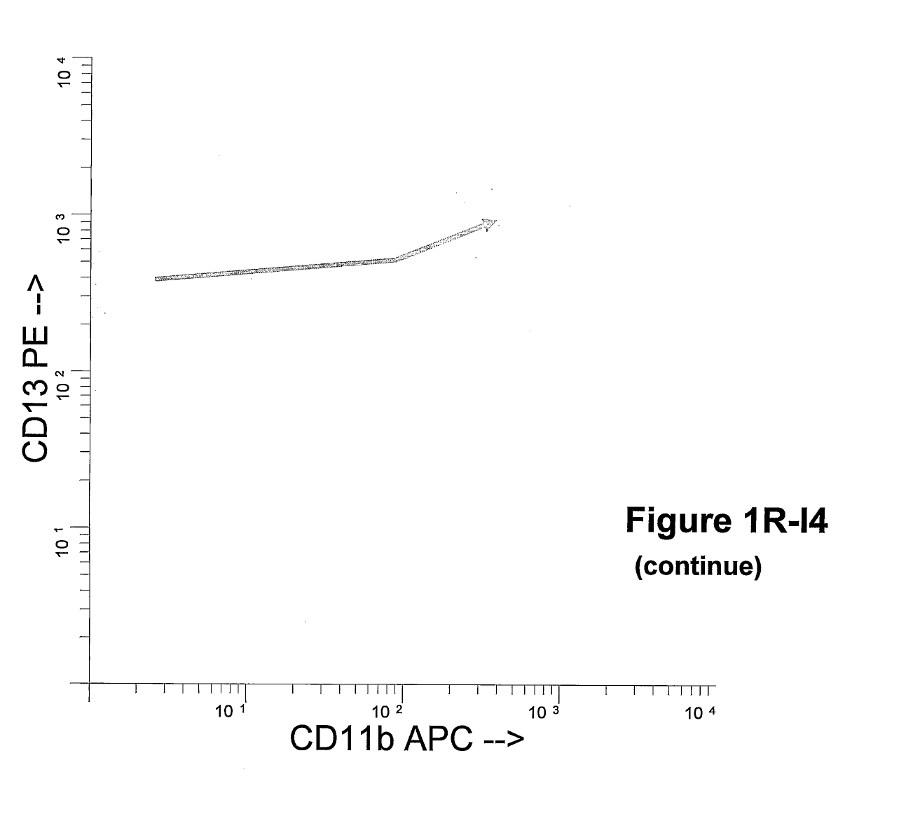

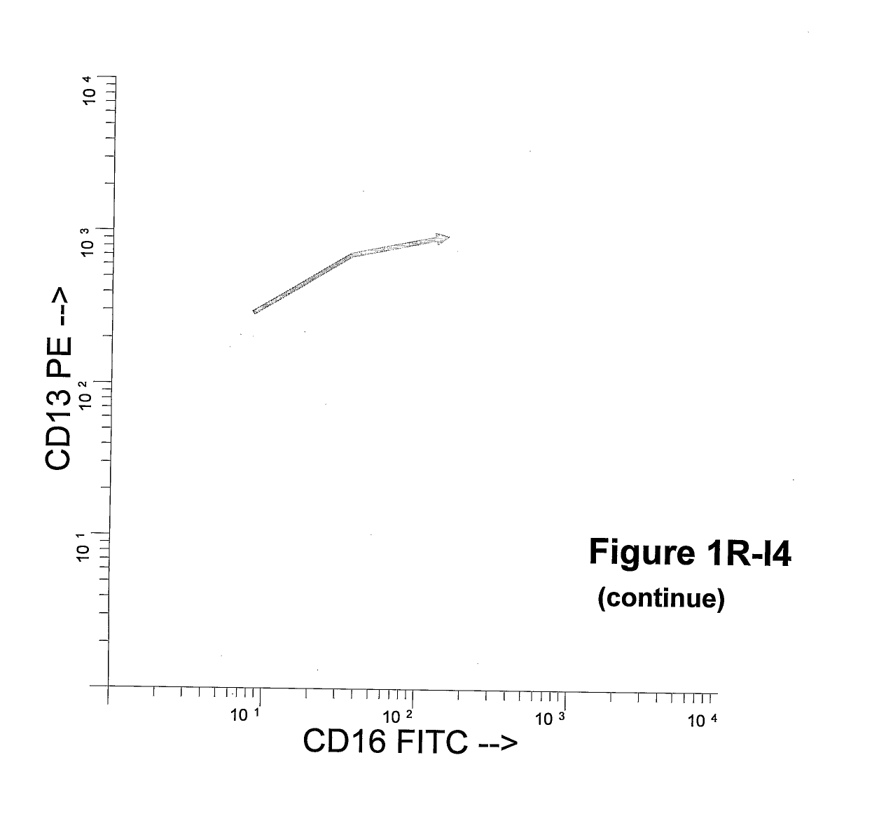

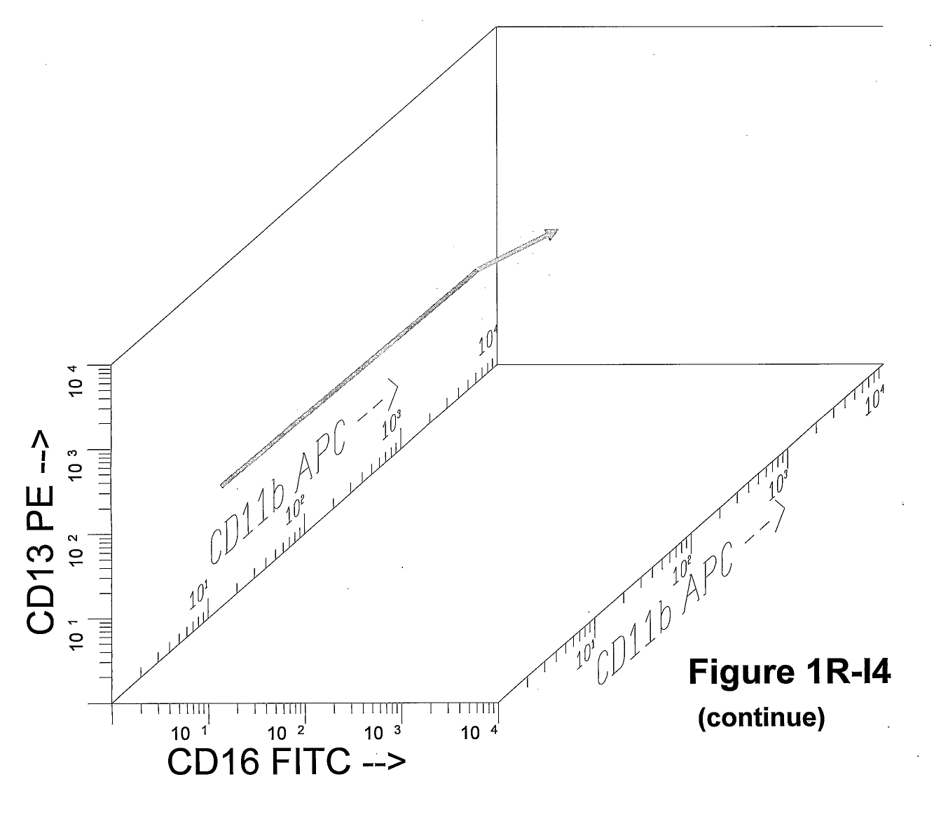

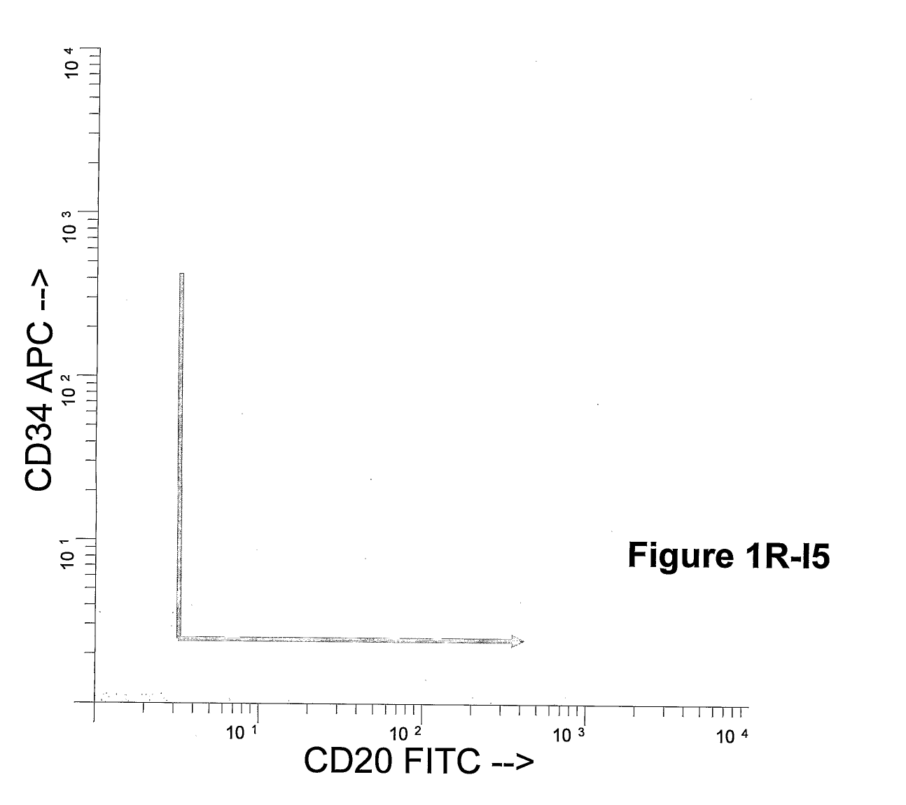

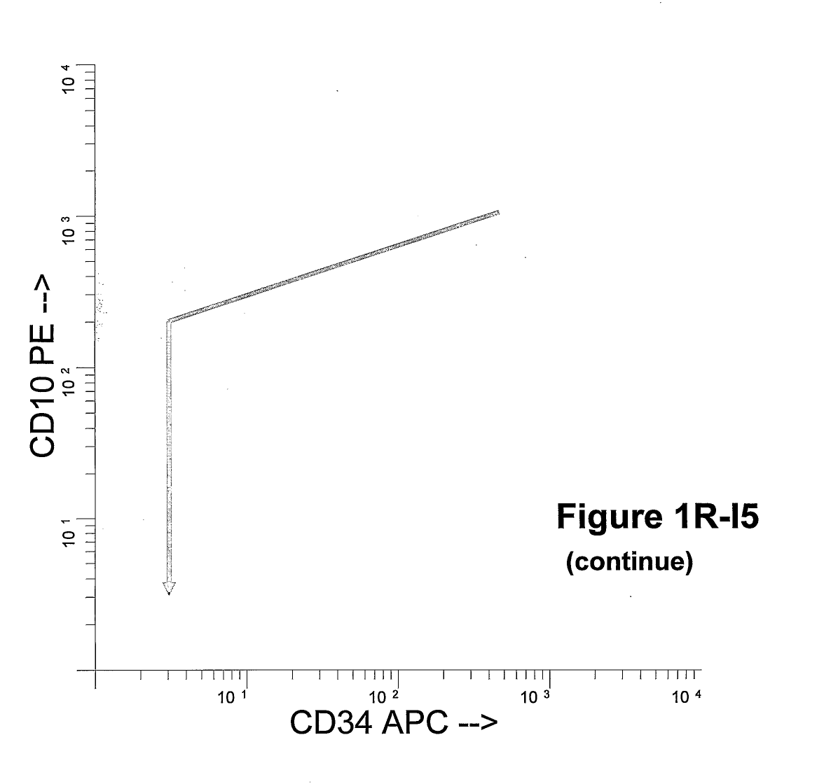

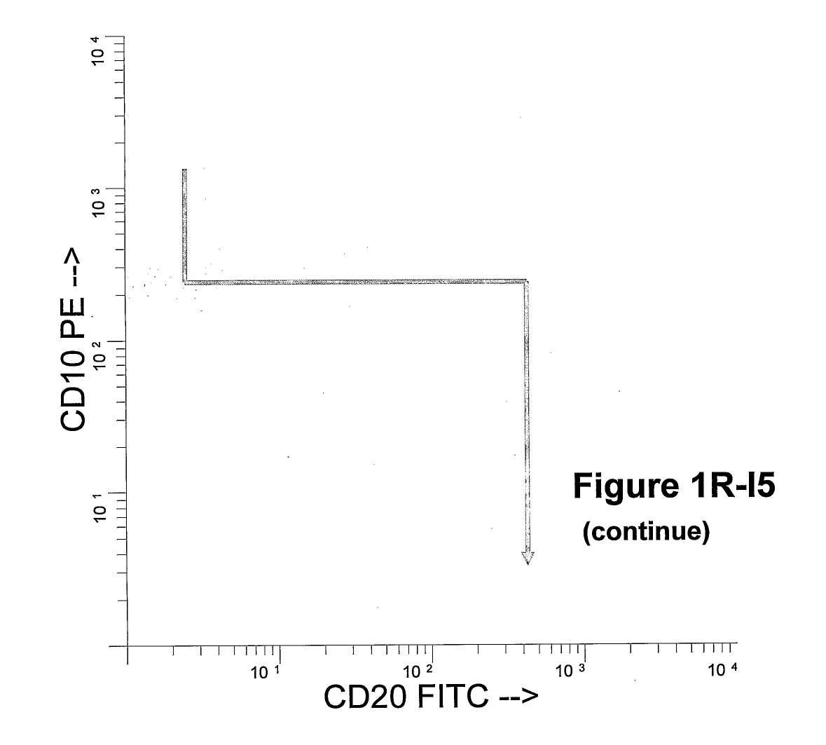

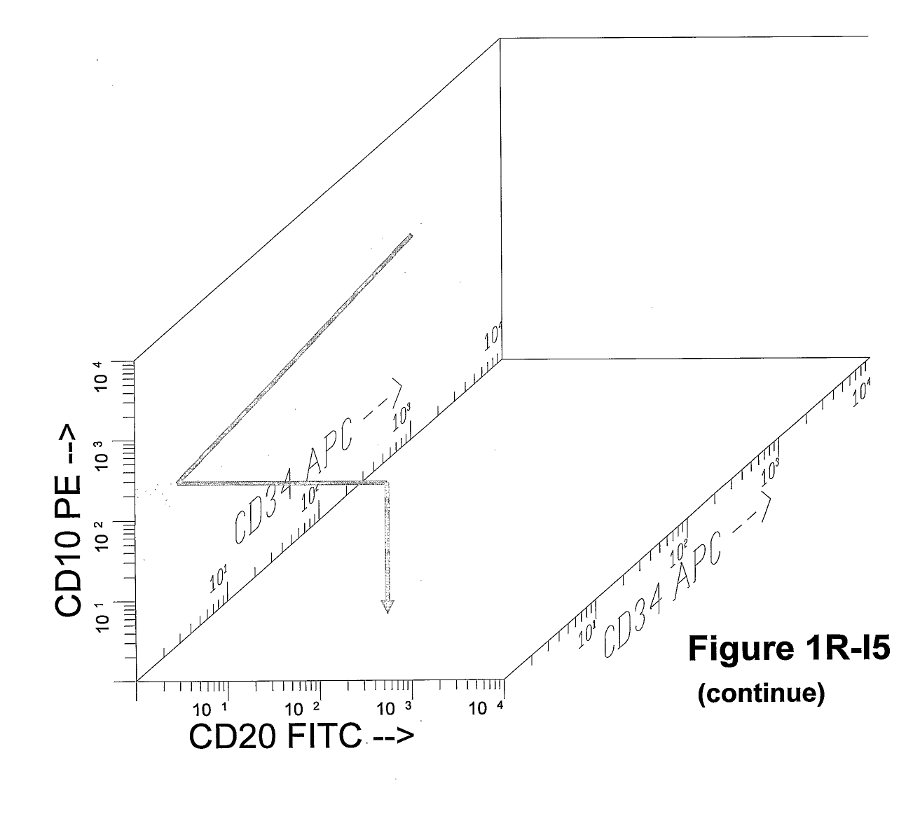

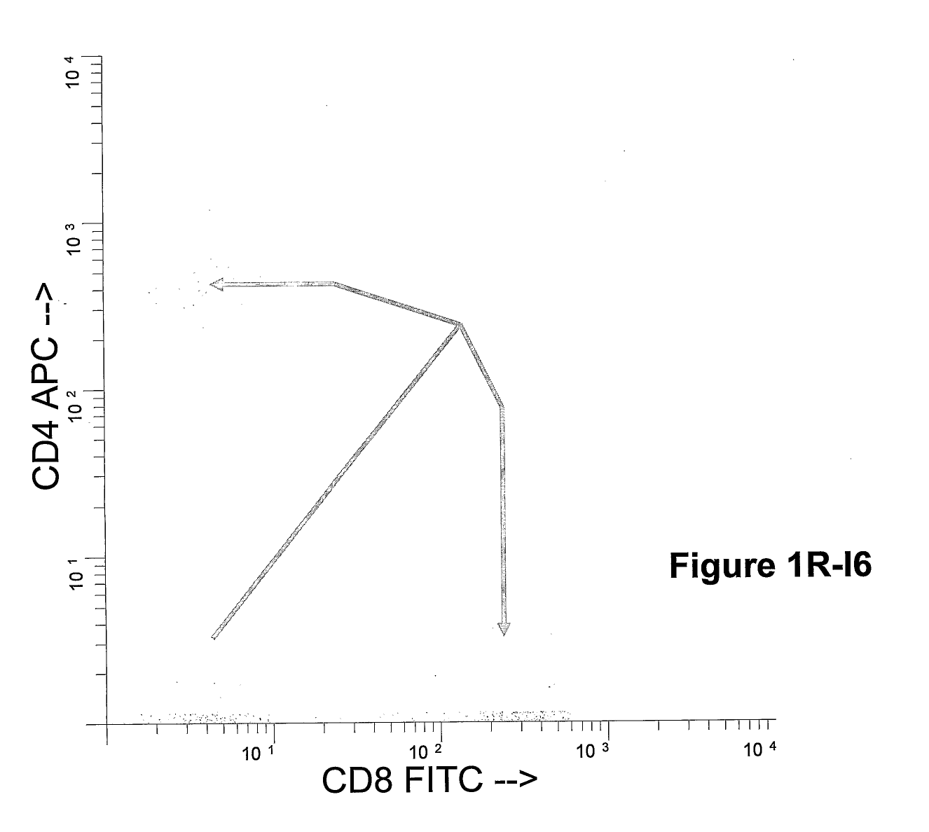

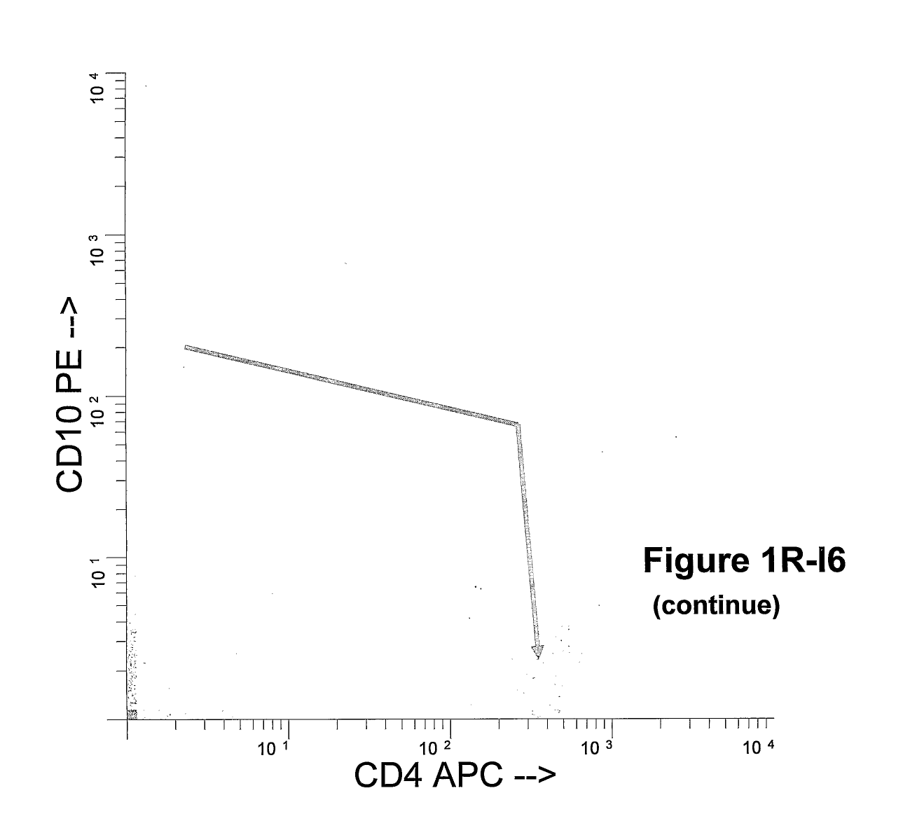

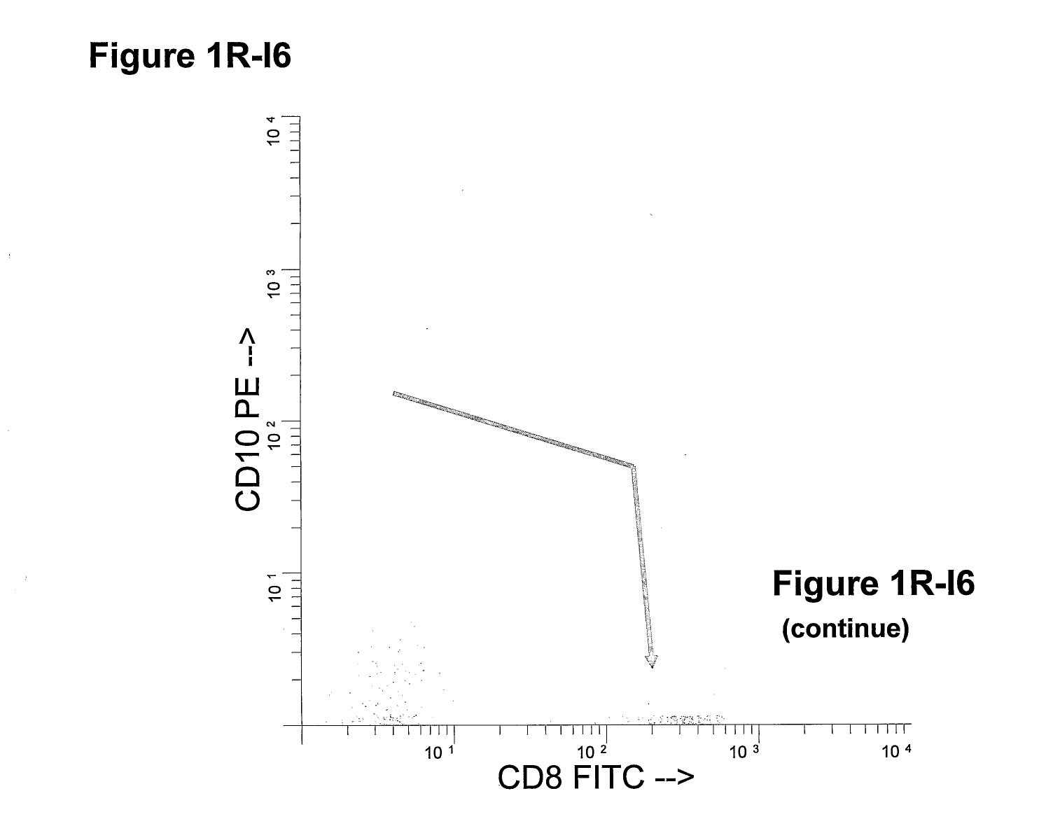

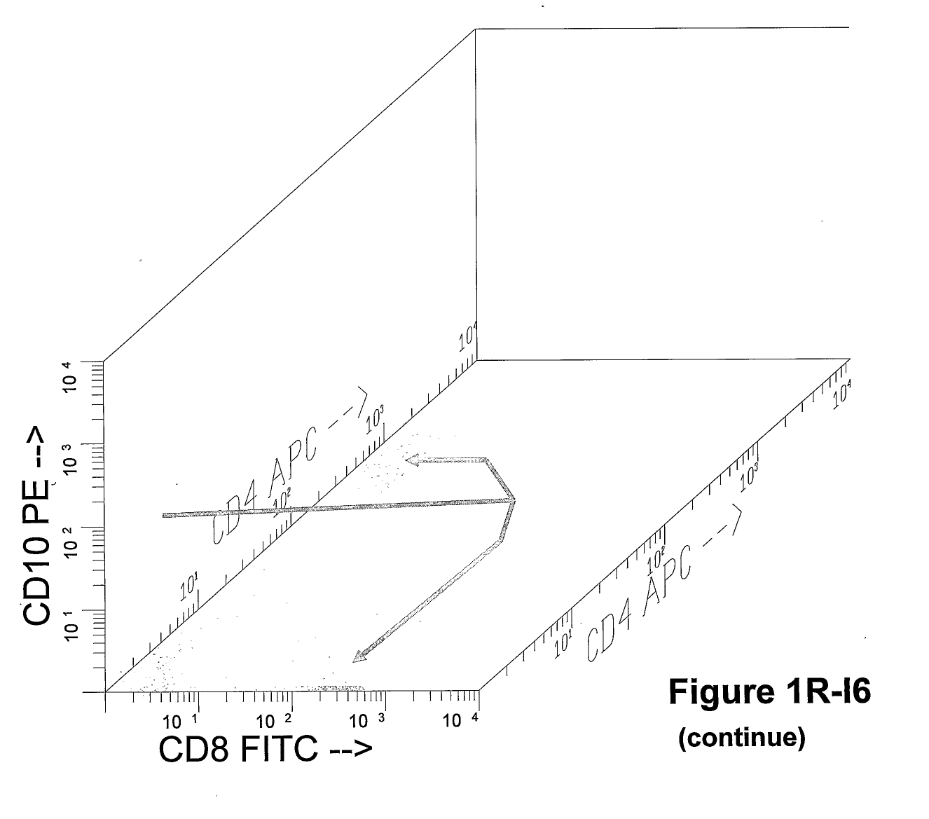

In the lymphocyte lineage, pluripotent stem cells are differentiated into lymphocyte lineage stem cells, and divided into B lymphocytic and T lymphocytic cells. In addition, there is another lineage which is differentiated into natural killer cells. The B lymphocytic lineage includes differentiation into precursor B lymphocyte, pre-pre-B-cell, early-B-cell and the like, and further differentiation into intermediate-B-cell, mature B-cell, plasmacytoid-B-cell, plasma-cell and the like. The T lymphocytic lineage includes differentiation into precursor thymocyte, immature thymocyte, common thymocyte, mature thymocyte. There are other lineages, which are differentiated into helper/inducer T lymphocytes and suppressor/cytotoxic T lymphocyte from mature thymocyte. Accordingly, the present invention such as an agent, composition, system and method may be useful for treating and/or preventing abnormalities of these T lymphocytes and/or B lymphocytes. A schematic drawing relating to differentiation is shown in FIG. 1A. FIG. 1A depicts markers useful for identifying differentiation. For detailed information regarding differentiation, please refer to Koichi AKATSUKASA, Saishin-Igaku (Recent Medicine) 56(2), 15-23, 2001, which is hereby incorporated as reference in it's entirety.

(Identification of a Hematopoietic Cell)

Hereinafter, representative identification methods of hematopoietic cells are described.

a. Spleen Colony Formation Method:

A mouse irradiated with a fatal dose of radiation is injected with hematopoietic cells of a syngeneic mouse, and a protrusion (colony) on the surface of spleen is observed on about Day 8-14. Each colony consists of a variety of blood cells, and a single colony is derived from a single stem cell, and the mother cell thereof, which forms spleen colony, is called CFU-S (colony forming unit in the spleen). Cells forming spleen colony formed on Day 8 (Day 8 CFU-S), consists mainly of erythroblasts, whereas spleen colony formed on Day 12 (Day 12 CFU-S) further include granulocytes and megalocytes in addition to erythroblasts and B lymphocytes, and has a potent proliferation capability, and is derived from pluripotent stem cells. Day 12 CFU-S is used as an indicator for pluripotent stem cells. Furthermore, hematopoietic stem cells remaining after 5-fluoro-uracil (5-FU), an anticancer agent, is administered, has extremely high proliferation potential, and is thus called pre-CFU-S, a parent cell of CFU-S. Day 12 CFU-S, which was believed to be an indicator for undifferentiated hematopoietic stem cells when it was first identified, is a heterologous cell population, and thus is not always an indicator for undifferentiated hematopoietic stem cells.

b. Long Term Bone Marrow Reconstruction Capability

This is a method for observing whether or not long term maintenance is possible by reconstructing the hematopoietic system of mice treated with fatal dose of radiation, with cells transplanted thereto. At present, this is the most reliable method for observing pluripotency and autoreplication of hematopoietic stem cells. Expression of neomycin resistant gene, sex chromosome including male and female, syngeneic mouse and the like, are used as a marker therefor. It was difficult to quantify by the present method, however, recently, a method has been developed wherein recipient hematopoietic cells are transplanted together with donor hematopoietic cells, and the ratio of reconstruction (so called competitive repopulation assay), is used. In humans, it is difficult to design a transplantation experimental system in vivo as in mice, Scid-hu mice are used, in which human hematopoietic stem cells have been transplanted into to scid mice, an immunodeficient mice, having no immunoreaction due to lack of lymphocytes. In this lineage, human hematopoietic machinery can be maintained for longer period of time in mice.

c. In Vitro Colony Method

This is a method for inferring the number and properties of a hematopoietic stem cells from a cell population (colony) formed by culturing hematopoietic cells (for example, bone marrow cells, spleen cells and the like) in a semi-solid medium such as methyl cellulose, soft agar or the like, in the presence of a variety of cytokines. Through analyses of these colonies, it has been possible to observe or determine the differentiation/proliferation processes of a variety of hematopoietic precursor cells and hematopoietic stem cells in vitro. Mixed colonies (CFU-Mix, CFU-GEMM) and colonies with high differentiation potential (HPP-CFC: high proliferative potential colony forming cells) are more undifferentiated than cells forming single lineage colonies such as CFU-GM, BFU-E and the like. Further, blast cells colony forming cells (CFU-blast) is recognized to be undifferentiated. This method allows the analysis and understanding of differentiation/maturation processes of hematopoietic stem cells and precursor cells in vitro. Further, more recently, single cell culture of hematopoietic stem cell in serum-free culture has been used to infer the action of a variety of cytokines relating to hematopoiesis.

d. Co-Culture System with Stromal Cells

Differentiation/maturation of hematopoietic cells is closely related to hematopoietic minor circumstances. In 1977, Dexter et al. has established a method where hematopoietic stem cells can be maintained several months or more on myeloid stromal cells (Dexter culture method or system). Thereafter, a number of stromal cell lines for maintaining hematopoiesis have been established, and have allowed the reconstruction of hematopoietic minor circumstances in vitro. This culture system causes hematopoietic precursor cells to loose their colony forming potential during early stage, whereas undifferentiated hematopoietic stem cells can maintain colony forming potential or bone marrow reconstruction potential for longer periods of time. Therefore, this method may be used for measuring undifferentiated hematopoietic stem cell activity. In humans, it is difficult to use in vivo system, and thus a cell which can maintain colony forming potential for long period of time on stromal cells is used as LTC-IC (Long term culture-initiating cells) which are indicative of an immature hematopoietic stem cell.

As such, in the diagnosis of the present invention, methods known in the art may be used for assisting diagnosis and detection.

As used herein, the term “expression” of a gene, a polynucleotide, a polypeptide, or the like, indicates that the gene or the like is affected by a predetermined action in vivo to be changed into another form. Preferably, the term “expression” indicates that genes, polynucleotides, or the like are transcribed and translated into polypeptides. In one embodiment of the present invention, genes may be transcribed into mRNA. More preferably, these polypeptides may have post-translational processing modifications. In other preferable embodiments, polypeptides thus expressed may be CD antigens with sugar chain modification. In the present invention, it is understood that expression includes both transcription and translation. Accordingly, when confirming the expression of a gene of a CD antigen of the present invention, it should be understood that the presence of both or either of the transcript and translate is to be confirmed.

As used herein, the term “expression” can be indicated by fluorescence intensity (FI) when using directly labeled antibody (for example, fluorescein isothyocyanate: FITC labeled CD antibody, phycoerythrin: PE labeled CD antibody, peridinin chlorophyll protein: PerCP labeled CD antibody, allophycocyanin: APC labeled CD antibody, and the like), primary antibodies (for example, biotinylated anti-CD antibody) and secondary antibodies (for example, phycoerythrin (PE) labeled streptoavidin). Such indication can be made according to an absolute or relative level.

The expression intensity of an mRNA level may be determined by using expression analysis with RT-PCR or microarray. When using RT-PCR, a densitometer is used for relative quantification, and if more detailed quantification is necessary, a microarray may be used in which HRPT, which is a house keeping gene, is used as reference, and expression less than that of HRPT is made negative, equal is made to weak positive, and expression greater than that of HRPT is made positive (strong positive), which may be processed statistically.

As used herein, the term “expression level” refers to a level of polypeptide or mRNA in a cell of interest, to be expressed therein. Such expression levels may be measured by the use of any appropriate method including immunological determination methods such as ELISA method, RIA method, fluorescence antibody method, Western blot method, immunohistochemistry method, and the like, to determine protein (expression) level of the polypeptide used in the present invention. In addition, any appropriate method including molecular biological determination methods such as Northern blotting, dot blotting, PCR and the like may also be used to determine expression levels at the mRNA level of the polypeptide used in the present invention. The term “change in expression level” as used herein, refers to increase or decrease of expression amount at protein or mRNA level of the polypeptide or mRNA used in the present invention which may be determined or evaluated by any appropriate method as described herein including immunological or molecular biological methods. Expression levels may be evaluated by absolute or relative levels.

As used herein, the term “interaction” with reference to two substances, means that one substance influences the other substance via forces (e.g., intermolecular forces (van der Waals force), hydrogen bonding, hydrophobic interactions, or the like). Typically, two substances interacting with each other interact in the manner of association or binding. Accordingly, when using an agent specific to a marker in order to confirm the presence of the marker, the presence of the marker can be confirmed by determining the interaction of the agent and the marker.

As used herein, the term “binding” means the physical or chemical interaction between two proteins or compounds or associated proteins or compounds or combinations thereof. Binding includes ionic, non-ionic, hydrogen, van der Waals, hydrophobic interactions, etc. A physical interaction (binding) can be either direct or indirect. Indirect interactions may be through or due to the effects of another protein or compound. Direct binding refers to interactions that do not take place through, or due to, the effect of another protein or compound, but instead are without other substantial chemical intermediates.

As used herein, the term “probe” refers to any substance which is used as a tool for search, in a biological experiment such as in vitro and/or in vivo screening and the like, and includes, but is not limited to, a nucleic acid molecule comprising a specific base sequence, or a peptide comprising a particular amino acid sequence, and the like.

Nucleic acid molecules normally used as a probe include

Examples of a nucleic acid molecule as a common probe include one having a nucleic acid sequence having a length of at least 8 contiguous nucleotides, which is homologous or complementary to the nucleic acid sequence of a gene of interest. Such a nucleic acid sequence may be preferably a nucleic acid sequence having a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 25 contiguous nucleotides, a length of at least 30 contiguous nucleotides, a length of at least 40 contiguous nucleotides, or a length of at least 50 contiguous nucleotides. A nucleic acid sequence used as a probe includes a nucleic acid sequence having at least 70% homology to the above-described sequence, more preferably at least 80%, and even more preferably at least 90% or at least 95%. Preferably, probes may be labeled.

Proteins typically used as a probe include, for example, but not limited to an antibody or derivative thereof. When using a protein as a probe, the protein can be directly or indirectly labeled with a labeling agent such as a fluorophore or radioisotope.

As used herein, the term “label” refers to an entity (for example, substance, energy, electromagnetic wave) for distinguishing a molecule or substance of interest from others. Such labeling methods include, but are not limited to, for example, radioisotope (RI) methods, fluorescence methods, biotin methods, chemiluminescence methods, and the like. When the above-mentioned nucleic acid fragments and oligonucleotides exhibiting complementarity thereto are labeled by a fluorescence method, fluorophores having different fluorescence emission local maximum wavelength to each other are used for labeling the same. The difference of fluorescence emission local maximum wavelength is preferably 10 nm or more. Fluorophores may be any moiety or entity which may be bound to the base portion of a nucleic acid, and include, but are not limited to: cyanin dye (for example, CyDye™ series including Cy3, Cy5 and the like), rhodamine 6G reagents, N-acetoxy-N2-acetyl aminofluorene (AAF), AAIF (iodine derivative of AAF), and the like, which are preferably used. Fluorophores having 10 nm or greater in difference of fluorescence emission local maximum wavelength, include, for example, the combinations of: Cy5 and rhodamine 6G reagent; Cy3 and fluorescein; rhodamine 6G and fluorescein and the like. In the present invention, such labels may be used to modify subjects of detection such that means for detection can detect the subject. Such modification is known in the art, and those skilled in the art can carry out an appropriate method depending on the label and the subject of interest.

As used herein, the term “corresponding” amino acid or nucleic acid refers to an amino acid or nucleotide in a given polypeptide or polynucleotide molecule, which has, or is anticipated to have, a function similar to that of a predetermined amino acid or nucleotide in a polypeptide or polynucleotide as a reference for comparison. Particularly, in the case of enzyme molecules, the term refers to an amino acid which is present at a similar position in an active site and similarly contributes to catalytic activity. For example, in the case of antisense molecules, the term refers to a similar portion in an ortholog corresponding to a particular portion of the antisense molecule. In CD antigens of the present invention, a corresponding amino acid may be a phosphorylated site, for example. In another embodiment, in the CD antigens of the present invention, a corresponding amino acid may be an amino acid playing a role in dimerization. Such a “corresponding” amino acid or nucleic acid may extend over a region or domain having a certain range. Therefore, in this case, such a region or domain is herein referred to as a “corresponding” region or domain.

As used herein, the term “corresponding” gene (e.g., a polypeptide or polynucleotide molecule) refers to a gene in a given species, which has, or is anticipated to have, a function similar to that of a predetermined gene in a species as a reference for comparison. When there are a plurality of genes having such a function, the term refers to a gene having the same evolutionary origin. Therefore, a gene corresponding to a given gene may be an ortholog of the given gene. Therefore, genes corresponding to mouse CD antigen genes can be found in other animals (e.g. human, rat, pig, cow and the like). Such a corresponding gene can be identified by techniques well known in the art. Therefore, for example, a corresponding gene in a given animal can be found by searching a sequence database of the animal (e.g., human, rat) using the sequence of a reference gene (e.g., mouse CD antigen gene, etc.) as a query sequence.

As used herein the terms “cellular marker” or “cell marker” are interchangeably used to refer to any markers which are capable of identifying a cell. Typically, such cellular markers include, but are not limited to, proteins (for example, CD antigens and the like).

As used herein the term “CD antigen” refers to any antigen against a CD. As used herein, the term “CD” refers to an abbreviation of a cluster of differentiation, and groups of monoclonal antibodies against human leukocyte differentiation antigens, classified by its property.

The International Workshop has agreed that CD antigens should be classified by clustering mainly based on biochemical properties of an antigen to be recognized by the antibody (in particular molecular weight). This is called CD classification, whereby a number of specific leukocyte differentiation antigen recognizing monoclonal antibodies have been uniformly designated in the form of CD number (for example, CD1, CD2 and the like), where CD is followed by a number. Typical examples used herein are described in detail as follows:

CD1 (4.3-4.9): an MHC class I-like molecule (which is so-called one of MHC class Ib antigens) which forms non-covalent bonding with β2 microglobulin. This has antigen presenting ability against a portion of T lymphocytes. There are four types: CD1a, CD1b, CD1c and CD1d, each of which exhibits specific organ distribution.

CD2 (5): a transmembrane protein of the immunoglobulin family. It is LFA-3 receptor, and is involved in rosette formation with sheep erythrocytes. It is expressed in T lymphocytes and natural killer (NK) cells.

CD3 (1.6-2.6): signal transduction molecules associating with T lymphocyte antigen receptors. They consist of five molecular species (γ, δ, ε, ζ and η) and six molecules (γ, δ, ε2, and ζ2 (or alternatively ζ and η)) associated per molecule to form functional a antigen receptor complex. They are mainly expressed in T lymphocytes.

CD 4 (6.2): a molecule which functions as a co-receptor for T lymphocyte antigen receptor complex, by binding to MHC class II molecule on an antigen presenting cell. It is expressed in helper T lymphocytes restricted to MHC class II.

CD5: T-cell surface glycoprotein CD5 (Lymphocyte glycoprotein T1/Leu-1).

CD7: T-cell antigen CD7 (Gp40) (T-cell leukemia antigen) (Tp41) (Leu-9).

CD8 (6.4) a dimer protein bound by S—S bonding between an α chain and a β chain. It binds to MHC class I molecules on antigen presenting cells, and functions as a T lymphocyte antigen receptor complex. It is expressed in killer T lymphocyte restricted to MHC class I.

CD10: Neprilysin (EC 3.4.24.11) (Neutral endopeptidase) (NEP) (Enkephalinase) (Common acute lymphocytic leukemia antigen) (CALLA).

CD11 (18): α chain of β2 integrin (a dimer consisting of an α chain and a β chain) which is an adhesive factor of leukocytes. It functions as a receptor for ICAM-1, C3bi, fibrinogen and the like, and is involved in adhesion, migration, phagocytosis and the like of leukocytes. There are three types including a(LFA-1), b(Mac-1), and c(p150/90). It is extensively expressed in monocytes, granulocytes, lymphocytes and the like.

CD13: Aminopeptidase N (EC 3.4.11.2) (Microsomal aminopeptidase) (Gp150).

CD14: Monocyte differentiation antigen CD14 (Myeloid cell-specific leucine-rich glycoprotein) (LPS receptor).

CD15: non-protein, sialyl Lewis (sLE).

CD16 (5-7): Low affinity immunoglobulin γ Fc region receptor III-A and III-B. There are two types: transmembrane format and lipid binding format; the former is expressed in NK cells and mediates antibody dependent cytotoxic activity.

CD19: B-lymphocyte antigen CD19 (B-lymphocyte surface antigen B4) (Leu-12).

CD20: B-lymphocyte antigen CD20 (B-lymphocyte surface antigen B1) (Leu-16) (Bp35).

CD22: B-cell receptor CD22 (Leu-14) (B-lymphocyte cell adhesion molecule) (BL-CAM).

CD23 (4.5): Low affinity immunoglobulin ε Fc receptor (Lymphocyte IgE receptor) (Fc-epsilon-RII) (BLAST-2). It is a transmembrane protein having lectin domain in extracellular portion, and a portion thereof is also present as a liberated form in the blood, and binds to IgE. It is expressed in lymphocytes, monocytes, platelets and the like.

CD33: bone marrow cellular surface antigen CD 33 (Gp 67);

CD34 (10.5-12): Transmembrane glycoprotein, which is selectively expressed in hematopoietic stem cells, and thus is commonly used as a marker for the identification or separation thereof.

CD45 (18-20): a large transmembrane protein having a number of sugar chains, and a tyrosine dephosphorylation active domain in the cell. It is a major protein expressed in all leukocytes, and is related to signal transduction via tyrosine kinase from a variety of membrane receptors. There are at least three types of isoforms including RO, RA and RB, which exhibit different distribution patterns.

TdT: Terminal deoxy nucleotide transferase (Terminal Transferase; available from BD Pharming and the like)

FMC7: It is a membrane glycoprotein of 105 kDa, and expressed in a subset of B lymphocytes. More than 50% of peripheral B lymphocytes of normal adults have this FMC 7 antigen. Available from Becton Dickinson Immunocytometry Systems.

HLA-DR: A type of human leukocyte antigen. HLA antigens are roughly classified into two groups: Class I antigen and Class II antigen. Class I antigens include, but are not limited to, for example, HLA-A, HLA-B, HLA-C, HLA-E, HLA-F, HLA-G and the like. Class I antigens are expressed in almost all nucleated cells. Class I antigens form a complex with a peptide produced in a cell, and the complex is presented to antigen specific T lymphocyte receptor of CD8 positive cytotoxic T lymphocytes. Class II antigens include, for example, HLA-DR, HLA-DQ, HLA-DP and the like.

The antigens may be commercially available from, specifically, BD Biosciences Pharmingen (USA; http://www.bdbiosciences.com), Beckton Dickinson Immuno cytometry Systems (San Jose, Calif., USA) and the like.

Whether a CD antigen is specific to a cell lineage, may be determined by observing an increase or decrease in the expression pattern thereof. The method includes the following steps: cells are classified into lymphocytes, monocytes, granulocytes, platelets, erythrocytes and the like depending on the size and intracellular structures thereof. Any CD antibody is used to detect whether or not such CD antibody reacts with each cell lineage to emit fluorescence. According to the present invention, it should be understood that such CD antigens other than the combination of the exemplified CD antigens may be used to identify a cell lineage (T lymphocytes, B lymphocytes, platelets, erythrocytes, monocytes and the like) for allowing a variety of analyses.

Description of the features of each CD antigen and gene sequence thereof is as follows:

| CD | Swiss-Prot | | MIM | Gene | |
|---|---|---|---|---|---|
| Number | Entry name | AC Nb | Number | name(s) | Name(s) for the protein |
| CD1a | CD1A_HUMAN | P06126 | 188370 | CD1A | T-cell surface glycoprotein CD1A (T-cell surface antigen T6/Leu-5) (HTA1 thymocyte antigen) |
| CD1b | CD1B_HUMAN | P29016 | 188360 | CD1B | T-cell surface glycoprotein CD1B |
| CD1c | CD1C_HUMAN | P29017 | 188340 | CD1C | T-cell surface glycoprotein CD1C |
| CD1d | CD1D_HUMAN | P15813 | 188410 | CD1D | T-cell surface glycoprotein CD1D (R3G1) |
| CD1e | CD1E_HUMAN | P15812 | 188411 | CD1E | T-cell surface glycoprotein CD1E (R2G1) |
| CD2 | CD2_HUMAN | P06729 | 186990 | CD2 | T-cell surface antigen CD2 (T-cell surface antigen T11/Leu-5) (LFA-2) (LFA-3 receptor) (Erythrocyte receptor) |
| | | | | | (Rosette receptor) |
| CD3d | CD3D_HUMAN | P04234 | 186790 | CD3D; T3D | T-cell surface glycoprotein CD3 delta chain (T-cell receptor T3 delta chain) |
| CD3e | CD3E_HUMAN | P07766 | 186830 | CD3E; T3E | T-cell surface glycoprotein CD3 epsilon chain (T-cell surface antigen T3/Leu-4 epsilon chain) |
| CD3g | CD3G_HUMAN | P09693 | 186740 | CD3G; T3G | T-cell surface glycoprotein CD3 gamma chain (T-cell receptor T3 gamma chain) |
| CD3z | CD3Z_HUMAN | P20963 | 186780 | CD3Z; T3Z | T-cell surface glycoprotein CD3 zeta chain (T-cell receptor T3 zeta chain) |
| CD4 | CD4_HUMAN | P01730 | 186940 | CD4 | T-cell surface glycoprotein CD4 (T-cell surface antigen T4/Leu-3) |
| CD5 | CD5_HUMAN | P06127 | 153340 | CD5 | T-cell surface glycoprotein CD5 (Lymphocyte glycoprotein T1/Leu-1) |
| CD6 | CD6_HUMAN | P30203 | 186720 | CD6 | T-cell differentiation antigen CD6 (T12) (Tp120) |
| CD7 | CD7_HUMAN | P09564 | 186820 | CD7 | T-cell antigen CD7 (Gp4D) (T-cell leukemia antigen) (Tp41) (Leu-9) |
| CD8a | CD8A_HUMAN | P01732 | 186910 | CD8A; MAL | T-cell surface glycoprotein CD8 alpha chain (T-lymphocyte differentiation antigen T8/Leu-2) (Lyt-2) |
| CD8b | CD8B_HUMAN | P10966 | 186730 | CD8B1 | T-cell surface glycoprotein CD8 beta chain (Lyt-3) |
| CD9 | CD9_HUMAN | P21926 | 143030 | CD9; MIC3 | CD9 antigen (p24) (Leukocyte antigen MIC3) |
| CD10 | NEP_HUMAN | P08473 | 120520 | MME; EPN | Neprilysin (EC 3.4.24.11) (Neutral endopeptidase) (NEP) (Enkephalinase) (Common acute lymphocytic leukemia |
| | | | | | antigen) (CALLA) |
| CD11a | ITAL_HUMAN | P20701 | 153370 | ITGAL; CD11A | Integrin alpha-L (Leukocyte adhesion glycoprotein LFA-1 alpha chain) |
| CD11b | ITAM_HUMAN | P11215 | 120980 | ITGAM; CD11B | Integrin alpha-M (Cell surface glycoprotein MAC-1 alpha subunit) (CR-3 alpha chain) (Leukocyte adhesion |
| | | | | | receptor Mo1) (Neutrophil adherence receptor) |
| CD11c | ITAX_HUMAN | P20702 | 151510 | ITGAX; CD11C | Integrin alpha-X (Leukocyte adhesion glycoprotein p150, 95 alpha chain) (Leukocyte adhesion receptor p150, 95) |
| | | | | | (Leu M5) |
| CD11d | ITAD_HUMAN | Q13349 | 602453 | ITGAD; CD11D | Integrin alpha-D (LeukoIntegrin alpha D) |
| CDw12 | N.A. | N.A. | N.A. | N.A. | [Deleted] |
| CD13 | AMPN_HUMAN | P15144 | 151530 | ANPEP; PEPN | Aminopeptidase N (EC 3.4.11.2) (Microsomal aminopeptidase) (Gp150) |
| CD14 | CD14_HUMAN | P08571 | 158120 | CD14 | Monocyte differentiation antigen CD14 (Myeloid cell-specific leucine-rich glycoprotein) (LPS receptor) |
| CD15 | N.A. | N.A. | N.A. | N.A. | [Not a protein: Sialyl Lewis (sLE)] |
| CD16a | FCG3A_HUMAN | P08637 | 146740 | FCGR3A; FCG3 | Low affinity immunoglobulin gamma Fc region receptor III-A (IgG FC Receptor III-2) (Fc-gamma RIII-alpha) |
| | | | | | (FcRIII) |
| CD16b | FCG3B_HUMAN | O75015 | N.A. | FCGR3B; FCG3 | Low affinity immunoglobulin gamma Fc region receptor III-B (IgG FC Receptor III-1) (Fc-gamma RIII-beta) |
| | | | | | (FcRIII) |
| CDw17 | N.A. | N.A. | N.A. | N.A. | [Not a protein: Lactosylceramide] |
| CD18 | ITB2_HUMAN | P05107 | 116920 | IGTB2; CD18 | Integrin beta-2 (Cell surface adhesion glycoproteins LFA-1/CR3/p150, 95 beta-subunit) |
| CD19 | CD19_HUMAN | P15391 | 107265 | CD19 | B-lymphocyte antigen CD19 (B-lymphocyte surface antigen B4) (Leu-12) |
| CD20 | CD20_HUMAN | P11836 | 112210 | MS4A2; CD20 | B-lymphocyte antigen CD20 (B-lymphocyte surface antigen B1) (Leu-16) (Bp35) |
| CD21 | CR2_HUMAN | P20023 | 120650 | C3DR; CR2 | Complement receptor type 2 (CR2) (Complement C3D receptor) (Epstein-Barr virus receptor) |
| CD22 | CD22_HUMAN | P20273 | 107266 | CD22 | B-cell receptor CD22 (Leu-14) (B-lymphocyte cell adhesion molecule) (BL-CAM) |
| CD23 | FCER2_HUMAN | P06734 | 151445 | FCER2; IGEBF | Low affinity immunoglobulin epsilon Fc receptor (Lymphocyte IgE receptor) (Fc-epsilon-RII) (BLAST-2) |
| CD24 | CD24_HUMAN | P25063 | 600074 | CD24 | Signal transducer CD24 |
| CD25 | IL2RA_HUMAN | P01589 | 147730 | IL2RA | Interleukin-2 receptor alpha chain (IL-2 receptor alpha subunit) (p55) (Tac antigen) |
| CD26 | DPP4_HUMAN | P27487 | 102720 | DPP4; ADCP2 | Dipeptidyl peptidase IV (EC 3.4.14.5) (DPP IV) (Tp103) (T-cell activation antigen CD26) (Adenosine deaminase |
| | | | | | complexing protein 2) |
| CD27 | TNR7_HUMAN | P26842 | 186711 | TNFRSF7; CD27 | Tumor necrosis factor receptor superfamily member 7 (CD27L receptor) T-cell activation antigen CD27 (T14) |
| CD28 | CD28_HUMAN | P10747 | 186760 | CD28 | T-cell-specific homodimer surface protein CD28 (Tp44) |
| CD29 | ITB1_HUMAN | P05556 | 135630 | ITGB1; FNRB | Integrin beta-1 (Fibronectin receptor beta subunit) |
| CD30 | TNR8_HUMAN | P28908 | 153243 | TNFRSF8; CD30 | Tumor necrosis factor receptor superfamily member 8 (CD30L receptor) (Lymphocyte activation antigen CD30) (Ki- |
| | | | | | 1) |
| CD31 | PECA1_HUMAN | P16284 | 173445 | PECAM1 | Platelet endothelial cell adhesion molecule (PECAM-1) |
| CD32 | FCG2A_HUMAN | P12318 | 146790 | FCGR2A | Low affinity immunoglobulin gamma Fc receptor II-A (Fc-gamma RII-A) (FcRII-A) (IgG Fc receptor II-A) |
| | FCG2B_HUMAN | P31994 | 604590 | FCGR2B | Low affinity immunoglobulin gamma Fc receptor II-B (Fc-gamma RII-B) (FcRII-B) (IgG Fc receptor II-B) |
| | FCG2C_HUMAN | P31995 | N.A. | FCGR2C | Low affinity immunoglobulin gamma Fc receptor II-C (Fc-gamma RII-C) (FcRII-C) (IgG Fc receptor II-C) |
| CD33 | CD33_HUMAN | P20138 | 159590 | CD33 | Myeloid cell surface antigen CD33 (Gp67) |
| CD34 | CD34_HUMAN | P28906 | 142230 | CD34 | Hematopoietic progenitor cell antigen CD34 |
| CD35 | CR1_HUMAN | P17927 | 120620 | C3BR; CR1 | Complement receptor type 1 (C3b/C4b receptor) |
| CD36 | CD36_HUMAN | P16671 | 173510 | CD36; GP3B | Platelet glycoprotein IV (GP IV)) GP IIIb) (CD36 antigen) |
| CD37 | CD37_HUMAN | P11049 | 151523 | CD37 | Leukocyte antigen CD37 |
| CD38 | CD38_HUMAN | P28907 | 107270 | CD38 | ADP-ribosylcyclase 1 (EC 3.2.2.5) (Cyclic ADP-ribose hydrolase 1) (Lymphocyte differentiation antigen CD38) |
| | | | | | (Acute lymphoblastic leukemia cells antigen CD38) (T10) |
| CD39 | ENP1_HUMAN | P49961 | 601752 | ENTPD1; CD39 | Ectonucleoside triphosphate diphosphohydrolase 1 (EC 3.6.1.5) (NTPDase1) (Ecto-ATP diphosphohydrolase) |
| | | | | | (ATPDase) (Lymphoid cell activation antigen) (Ecto-apyrase) |
| CD40 | TNR5_HUMAN | P25942 | 109535 | TNFRSF5; CD40 | Tumor necrosis factor receptor superfamily member 5 (CD40L receptor) (B-cell surface antigen CD40) (Bp50) |
| CD41 | ITA2B_HUMAN | P08514 | 273800 | ITGAB; GP2B | Integrin alpha-IIB (Platelet membrane glycoprotein IIb) (GP IIb) |
| CD42a | GP1X_HUMAN | P14770 | 173515 | GP9 | Platelet glycoprotein IX (Gp1X) |
| CD42b | GP1BA_HUMAN | P07359 | 231200 | GP1BA | Platelet glycoprotein Ib alpha chain (Gp1Ba) |
| CD42c | GP1BB_HUMAN | P13224 | 138720 | GP1BB | Platelet glycoprotein Ib beta chain (Gp1Bb) |
| CD42d | GPV_HUMAN | P40197 | 173511 | GP5 | Platelet glycoprotein V (GpV) |
| CD43 | LEUK_HUMAN | P16150 | 182160 | SPN | Leukosialin (Leucocyte sialoglycoprotein) (Sialophorin) (Galactoglycoprotein) (GalGp) |
| CD44 | CD44_HUMAN | P16070 | 107269 | CD44; LHR | CD44 antigen (Phagocytic glycoprotein 1) (PGP-1) (Extracellular matrix receptor-III) (ECMR-III) (Gp90 |
| | | | | | lymphocyte homing/adhesion receptor) (Hermes antigen) (Heparan sulfate proteoglycan) (Epican) |
| CD45 | CD45_HUMAN | P08575 | 151460 | PTPRC; CD45 | Leukocyte common antigen (L-CA) (T200) (EC 3.1.3.48) |
| CD46 | MCP_HUMAN | P15529 | 120920 | MCP | Membrane cofactor protein (Trophoblast leucocyte common antigen) (TLX) |
| CD47 | CD47_HUMAN | Q08722 | 601028 | CD47 | Leukocyte surface antigen CD47 (Antigenic surface determinant protein OA3) (Integrin associated protein) (IAP) |
| CD48 | CD48_HUMAN | P09326 | 109530 | CD48; BCM1 | B-lymphocyte activation marker Blast-1 (BCM1 surface antigen) (Leucocyte antigen mem-102) (TCT.1) |
| CD49a | ITA1_HUMAN | P56199 | 192968 | ITGA1 | Integrin alpha-1 (Integrin VLA-1 alpha subunit) (Laminin and collagen receptor) |
| CD49b | ITA2_HUMAN | P17301 | 192974 | ITGA2 | Integrin alpha-2 (Integrin VLA-2 alpha subunit) (Platelet membrane glycoprotein Ia) (GP Ia) (Collagen |
| | | | | | receptor) |
| CD49c | ITA3_HUMAN | P26006 | 605025 | ITGA3 | Integrin alpha-3 (Integrin VLA-3 alpha subunit) (Galactoprotein b3) (Gapb3) |
| CD49d | ITA4_HUMAN | P13612 | 192972 | ITGA4 | Integrin alpha-4 (Integrin VLA-4 alpha subunit) |
| CD49e | ITA5_HUMAN | P08648 | 135620 | ITGA5; FNRA | Integrin alpha-5 (Integrin VLA-5 alpha subunit) (Fibronectin receptor alpha subunit) |
| CD49f | ITA6_HUMAN | P23229 | 147556 | ITGA6 | Integrin alpha-6 (Integrin VLA-6 alpha subunit) |
| CD50 | ICAM3_HUMAN | P32942 | 146631 | ICAM3 | Intercellular adhesion molecule-3 (ICAM-3) (ICAM-R) |
| CD51 | ITAV_HUMAN | P06756 | 193210 | ITGAV; VNRA | Integrin alpha-V (Vitronectin receptor alpha subunit) |
| CD52 | CD52_HUMAN | P31358 | 114280 | CD52; CDW52 | CAMPATH-1 antigen |
| CD53 | CD53_HUMAN | P19397 | 151525 | CD53; MOX44 | Leukocyte surface antigen CD53 |
| CD54 | ICAM1_HUMAN | P05362 | 147840 | ICAM1 | Intercellular adhesion molecule-1 (ICAM-1) (Major group rhinovirus receptor) |
| CD55 | DAF_HUMAN | P08174 | 125240 | DAF | Complement decay-accelerating factor |
| CD56 | NCA11_HUMAN | P13591 | 116930 | NCAM1; NCAM | Neural cell adhesion molecule 1 |
| | NCA12_HUMAN | P13592 | | | |
| CD57 | N.A. | N.A. | 151290 | | [Not a protein: Sulfated glucuronic acid complex; Leu-7] |
| CD58 | LFA3_HUMAN | P19256 | 153420 | CD58; LFA3 | Lymphocyte function-associated antigen 3 (Surface glycoprotein LFA-3) |
| CD59 | CD59_HUMAN | P13987 | 107271 | CD59 | CD59 glycoprotein (Membrane attack complex inhibition factor) (MACIF) (MAC-inhibitory protein) (MAC-IP) |
| | | | | | (Antigen MEM43) (Protectin) |
| CD60a | N.A. | N.A. | N.A. | N.A. | [Not a protein: Oligosaccharide present on gangliosides; GD3] |
| CD60b | N.A. | N.A. | N.A. | N.A. | [Not a protein: Oligosaccharide present on gangliosides: 9-0-acetyl-GD3] |
| CD60c | N.A. | N.A. | N.A. | N.A. | [Not a protein: Oligosaccharide present on gangliosides; 7-0-acetyl-GD3] |
| CD61 | ITB3_HUMAN | P05106 | 173470 | ITGB3; GP3A | Integrin beta-3 (Platelet membrane glycoprotein IIIa) (GP IIIa) |
| CD62E | LYAM2_HUMAN | P16581 | 131210 | SELE; ELAM1 | E-Selectin (Endothelial leukocyte adhesion molecule-1) (ELAM-1) (Leukocyte-endothelial cell adhesion molecule |
| | | | | | 2) (LECAM2) |
| CD62L | LYAM1_HUMAN | P14151 | 153240 | SELL; LYAM1 | L-Selectin (Lymph node homing receptor) (Leukocyte adhesion molecule-1) (LAM-1) (Leukocyte surface antigen |
| | | | | | leu-8) |
| CD62P | LYAM3_HUMAN | P16109 | 173610 | SELP; GMRP | P-Selectin (Granule membrane protein 140) (GMP-140) (Leukocyte-endothelial cell adhesion molecule 3) (LECAM3) |
| CD63 | CD63_HUMAN | P08962 | 155740 | CD63; MLA1 | CD63 antigen (Melanoma-associated antigen ME491) (Lysosome-associated membrane glycoprotein 3) (LAMP-3) |
| CD64 | FCGR1_HUMAN | P12314 | 146760 | FCGR1A; FCG1 | High affinity immunoglobulin gamma Fc receptor I (Fc-gamma RI) (FcRI) (IgG Fc receptor I) |
| CD65 | N.A. | N.A. | N.A. | N.A. | [Not a protein: Ceramide dodecasacharide 4c (VIM-2)] |
| CD66a | CEAM1_HUMAN | P13688 | 109770 | CEACAM1; BGP1 | Carcinoembryonic antigen-related cell adhesion molecule 1 (Biliary glycoprotein 1) (BGP-1) (Phosphoprotein |
| | | | | | Gp180-200) |
| CD66b | CEAM8_HUMAN | P31997 | 114890 | CEACAM8; CGMG | Carcinoembryonic antigen-related cell adhesion molecule 8 (Carcinoembryonic antigen CGMB) (Nonspecific cross- |
| | | | | | reacting antigen NCA-95) |
| CD66c | CEAM6_HUMAN | P40199 | 163980 | CEACAM6; NCA | Carcinoembryonic antigen-related cell adhesion molecule 6 (Normal cross-reacting antigen) |
| CD66d | CEAM3_HUMAN | P40198 | 114890 | CEACAM3; CGM1 | Carcinoembryonic antigen-related cell adhesion molecule 3 (Carcinoembryonic antigen CGM1) |
| CD66e | CEAM5_HUMAN | P06731 | 114890 | CEACAM5; CEA | Carcinoembryonic antigen-related cell adhesion molecule 5 (Carcinoembryonic antigen) (Meconium antigen 100) |
| CD66f | PSG1_HUMAN | P11464 | 176390 | PSG1; B1G1 | Pregnancy-specific beta-1-glycoprotein 1 (PSBG-1) (Pregnancy-specific beta-1 glycoprotein C/D) (PS-beta-C/D) |
| | | | | | (Fetal liver non-specific cross-reactive antigen-1/2) (FL-NCA-1/2) (PSG95) |
| CD67 | N.A. | N.A. | N.A. | N.A. | [Deleted: now CD66b] |
| CD68 | CD68_HUMAN | P34810 | 153634 | CD68 | Gp110 (Macrosialin) |
| CD69 | CD69_HUMAN | Q07108 | 107273 | CD69 | Early T-cell activation antigen p60 (Gp32/28) (Leu-23) (MLR-3) (Activation inducer molecule) (AIM) |
| CD70 | TNFL7_HUMAN | P32970 | 602840 | TNFSF7; CD27L | CD27 ligand (CD27-L) (Kl-24) |
| CD71 | TFR1_HUMAN | P02786 | 190010 | TFRC | Transferrin receptor protein 1 (TfR1) (TR) (TfR) (T9) |
| CD72 | CD72_HUMAN | P21854 | 107272 | CD72 | B-cell differentiation antigen CD72 (Lyb-2) |
| CD73 | 5NTD_HUMAN | P21589 | 129190 | NT5E; NT5; NTE | 5′-nucleotidase (EC 3.1.3.5) |
| CD74 | HG2A_HUMAN | P04233 | 142790 | CD74; DHLAG | HLA class II histocompatibility antigen, gamma chain (HLA-Dr antigens associated invariant chain) |
| CD75 | N.A. | N.A. | N.A. | N.A. | [Not a protein: Lactosamines] |
| CD75s | N.A. | N.A. | N.A. | N.A. | [Not a protein: Alpha-2, 6-slalylated lactosamines] |
| CDw76 | N.A. | N.A. | N.A. | N.A. | [Deleted: now CD75s] |
| CD77 | N.A. | N.A. | N.A. | N.A. | [Not a protein: Globotriaocylceramide (Gb3) (Burkitt's lymphoma associated antigen) (BLA)] |
| CDw78 | N.A. | N.A. | N.A. | N.A. | [Deleted] |
| CD79a | CD79A_HUMAN | P11912 | 112205 | CD79A; IGA; MB1 | B-cell antigen receptor complex associated protein alpha-chain (Ig-alpha) (MB-1 membrane glycoprotein) |
| | | | | | (Surface-IgM-associated protein) (Membrane-bound immunoglobulin associated protein) |
| CD79b | CD79B_HUMAN | P40259 | 147245 | CD79B; IGB; B29 | B-cell antigen receptor complex associated protein beta-chain (Ig-beta) (B-cell-specific glycoprotein B29) |
| | | | | | (immunoglobulin-associated b29 protein) |
| CD80 | CD80_HUMAN | P33681 | 112203 | CD80; CD28LG1 | T lymphocyte activation antigen CD80 (Activation B7-1 antigen) (B7) (BB1) |
| CD81 | CD81_HUMAN | P60033 | 186845 | CD81; TAPA1 | 26 kDa cell surface protein TAPA-1 |
| CD82 | CD82_HUMAN | P27701 | 600623 | CD82; KAI1; SAR2 | Inducible membrane protein R2 (C33 antigen) (IA4) (Metastasis suppressor Kangal 1) (Suppressor of |
| | | | | | tumorigenicity-6) |
| CD83 | CD83_HUMAN | Q01151 | 604534 | CD83 | Cell surface protein HB15 |
| CD84 | N.A. | N.A. | 604513 | CD84 | Leukocyte antigen CD84 (GR6) (Ly9-beta) |
| CD85a | LIRB3_HUMAN | O75022 | 604820 | LILRB3; LIR3 | Leukocyte immunoglobulin-like receptor subfamily B member 3 (Leucocyte immunoglobulin-like receptor 3) (LIR-3) |
| | | | | | (immunoglobulin-like transcript 5) (ILT-5) (Monocyte inhibitory receptor HL9) |
| CD85c | LIRB5_HUMAN | O75023 | 604814 | LILRB5; LIR8 | Leukocyte immunoglobulin-like receptor subfamily B member 5 (Leucocyte immunoglobulin-like receptor 8) (LIR-8) |
| CD85d | LIRB2_HUMAN | Q8N423 | 604815 | LILRB2; LIR2 | Leukocyte immunoglobulin-like receptor subfamily B member 2 (Leukocyte immunoglobulin-like receptor 2) (LIR-2) |
| | | | | | (immunoglobulin-like transcript 4) (ILT-4) (Monocyte/macrophage immunoglobulin-like receptor 10) (MIR-10) |
| CD85e | LIRA3_HUMAN | Q8N6CB | 604818 | LILRA3; LIR4 | Leukocyte immunoglobulin-like receptor subfamily A member 3 (Leukocyte immunoglobulin-like receptor 4) (LIR-4) |
| | | | | | (immunoglobulin-like transcript 6) (ILT-6) (Monocyte inhibitory receptor HM43/HM31) |
| CD85g | LIRA4_HUMAN | P59901 | 607517 | LILRA4; ILT7 | Leukocyte immunoglobulin-like receptor subfamily A member 4 (immunoglobulin-like transcript 7) (ILT-7) |
| CD85h | LIRA2_HUMAN | Q8N149 | 604812 | LILRA2; LIR7 | Leukocyte immunoglobulin-like receptor subfamily A member 2 (Leucocyte immunoglobulin-like receptor 7) (LIR-7) |
| | | | | | (immunoglobulin-like transcript 1) (ILT-1) |
| CD85i | LIRA1_HUMAN | O75019 | 604810 | LILRA1; LIR6 | Leukocyte immunoglobulin-like receptor subfamily A member 1 (Leucocyte immunoglobulin-like receptor 6) (LIR-6) |
| CD85j | LIRB1_HUMAN | Q8NHL6 | 604811 | LILRB1; LIR1 | Leukocyte immunoglobulin-like receptor subfamily B member 1 (Leukocyte immunoglobulin-like receptor 1) (LIR-1) |
| | | | | | (immunoglobulin-like transcript 2) (ILT-2) (Monocyte/macrophage immunoglobulin-like receptor 7) (MIR-7) |
| CD85k | LIRB4_HUMAN | Q8NHJ6 | 604821 | LILRB4; LIR5 | Leukocyte immunoglobulin-like receptor subfamily B member 4 (Leukocyte immunoglobulin-like receptor 5) (LIR-5) |
| | | | | | (immunoglobulin-like transcript 3) (ILT-3) (Moncoyte inhibitory receptor HM18) |
| CD86 | CD86_HUMAN | P42081 | 601020 | CD86; CD28LG2 | T lymphocyte activation antigen CD86 (Activation B7-2 antigen) (B70) (FUN-1) (BU63) |
| CD87 | UPAR_HUMAN | Q03405 | 173391 | PLAUR; UPAR | Urokinase plasminogen activator surface receptor (U-PAR) (Monocyte activation antigen mo3) |
| CD88 | C5AR_HUMAN | P21730 | 113995 | C5R1; C5AR | C5A anaphylatoxin chemotactic receptor (C5A-R) |
| CD89 | FCAR_HUMAN | P24071 | 147045 | FCAR; CD89 | Immunoglobulin alpha Fc receptor (IgA Fc receptor) |
| CD90 | THY1_HUMAN | P04216 | 188230 | THY1 | Thy-1 membrane glycoprotein |
| CD91 | LRP1_HUMAN | Q07954 | 107770 | LRP1; A2MR | Low density lipoprotein-related protein 1 (Alpha-2-macroglobulin receptor) |
| CDw92 | N.A. | N.A. | N.A. | N.A. | p70 (GR9) |
| CD93 | C1QR1_HUMAN | Q9NPY3 | 120577 | C1QR1; CD93 | Complement component C1q recepter (Complement component 1, q subcomponent, receptor 1) (C1qRp) (C1qR(p)) |
| | | | | | (C1q/MBL/SPA receptor) |
| CD94 | KLRD1_HUMAN | Q13241 | 602894 | KLRD1; CD94 | Natural killer cells antigen CD94 (Kp43) |
| CD95 | TNR6_HUMAN | P25445 | 134637 | TNFRSF6; APT1 | Tumor necrosis factor receptor superfamily member 6 (FASL receptor) (Apoptosis-mediating surface antigen FAS) |
| | | | | | (APO-1 antigen) |
| CD96 | TACT_HUMAN | P40200 | N.A. | CD96 | T-cell surface protein tactile |
| CD97 | CD97_HUMAN | P48960 | 601211 | CD97 | Leucocyte antigen CD97 |
| CD98 | 4F2_HUMAN | P08195 | 158070 | SLC3A2; MDU1 | 4F2 cell-surface antigen heavy chain (4F2hc) (Lymphocyte activation antigen 4F2 large subunit) |
| CD99 | MIC2_HUMAN | P14209 | 313470 | CD99; MIC2 | T-cell surface glycoprotein E2 (E2 antigen) |
| CD100 | SEM4D_HUMAN | Q92854 | 601866 | SEMA4D; CD100 | Semaphorin 4D (8B18) (A8) (GR3) |
| CD101 | N.A. | N.A. | 604516 | IGSF2 | BB27 (BA27) |
| CD102 | ICAM2_HUMAN | P13598 | 146630 | ICAM2 | Intercellular adhesion molecule-2 (ICAM-2) |
| CD103 | ITAE_HUMAN | P38570 | 604682 | ITGAE | Integrin alpha-E (Mucosal lymphocyte-1 antigen) (HML-1) |
| CD104 | ITB4_HUMAN | P16144 | 147557 | ITGB4 | Integrin beta-4 (Gp150) |
| CD105 | EGLN_HUMAN | P17813 | 131195 | ENG; END | Endoglin |
| CD106 | VCAN1_HUMAN | P19320 | 192225 | VCAM1; L1CAM | Vascular cell adhesion protein 1 (V-CAM 1) |
| CD107a | LAMP1_HUMAN | P11279 | 153330 | LAMP1 | Lysosome-associated membrane glycoprotein 1 (LAMP-1) |
| CD107b | LAMP2_HUMAN | P13473 | 309060 | LAMP2 | Lysosome-associated membrane glycoprotein 1 (LAMP-2) |
| CD108 | SEM7A_HUMAN | O75326 | 607961 | SEMA7A; SEMAL | Semaphorin 7A (Semaphorin L) (Sema L) (Semaphorin K1) (Sema K1) (John-Milton-Hargen human blood group Ag) |
| CD109 | N.A. | N.A. | N.A. | N.A. | Platelet activation factor (8A3) (7D1) (GR56) |
| CD110 | TPOR_HUMAN | P40238 | 159530 | MPL; TPOR | Thrombopoietin receptor (TPO-R) (Myeloproliferative leukemia protein) (C-mpl) |
| CD111 | PVR1_HUMAN | Q15223 | 600644 | PVRL1; PRR1 | Poliovirus receptor related protein 1 (Herpes virus entry mediator C) (HveC) (Nectin 1) |
| CD112 | PVR2_HUMAN | Q92692 | 600798 | PVRL2; PRR2 | Poliovirus receptor related protein 2 (Herpes virus entry mediator B) (HveB) (Nectin 2) |
| CD113 | N.A. | N.A. | N.A. | N.A. | Not yet assigned |
| CD114 | CSF3R_HUMAN | Q99062 | 138971 | CSF3R; GCSFR | Granulocyte colony stimulating factor receptor (G-CSF-R) |
| CD115 | CSF1R_HUMAN | P07333 | 164770 | CSF1R; FMS | Macrophage colony stimulating factor I receptor (CSF-1-R) (EC 2.7.1.112) (Fms proto-oncogene) (c-fms) |
| CD116 | CSF2R_HUMAN | P15509 | 306250 | CSF2RA; CSF2R | Granulocyte-macrophage colony-stimulating factor receptor alpha chain (GM-CSF-R-alpha) |
| CD117 | KIT_HUMAN | P10721 | 164920 | KIT | Mast/stem cell growth factor receptor (EC 2.7.1.112) (SCFR) (Proto-oncogene tyrosine-protein kinase KIT) (c- |
| | | | | | KIT) |
| CD119 | INGR1_HUMAN | P15260 | 107470 | IFNGR1 | Interferon-gamma receptor alpha chain |
| CD120a | TNR1A_HUMAN | P19438 | 191190 | TNFRSF1A; TNFR1 | Tumor necrosis factor receptor 1 (Tumor necrosis factor binding protein 1) (TBPI) (p60) (TNF-R1) (p55) |
| CD120b | TNR1B_HUMAN | P20333 | 191191 | TNFRSF1B; TNFR2 | Tumor necrosis factor receptor 2 (Tumor necrosis factor binding protein 2) (TBPII) (p80) (TNF-R2) (p75) |
| CD121a | IL1R1_HUMAN | P14778 | 147810 | IL1RA; IL1R1 | Interleukin-1 receptor, type 1 (IL-1R-1) (IL-1R-alpha) |
| CD121b | IL1R2_HUMAN | P27930 | 147811 | IL1RB; IL1R2 | Interleukin-1 receptor, type 2 (IL-1R-2) (IL-1R-beta) |
| CD122 | IL2RB_HUMAN | P14784 | 146710 | IL2RB | Interleukin-2 receptor beta chain (IL-2 receptor beta subunit) (p75) |
| CD123 | IL3RA_HUMAN | P26951 | 308385 | IL3RA | Interleukin-3 receptor alpha chain (IL-3R-alpha) |
| CD124 | IL4RA_HUMAN | P24394 | 147781 | IL4R | Interleukin-4 receptor alpha chain (IL-4R-alpha) |
| CD125 | IL5RA_HUMAN | Q01344 | 147851 | IL5RA; IL5R | Interleukin-5 receptor alpha chain (IL-5R-alpha) |
| CD126 | IL6RA_HUMAN | P08887 | 147880 | IL6R | Interleukin-6 receptor alpha chain (IL-6R-alpha) |
| CD127 | IL7RA_HUMAN | P16871 | 146661 | IL7R | Interleukin-7 receptor alpha chain (IL-7R-alpha) |
| CDw128a | CXCR1_HUMAN | P25024 | 146929 | IL8RA; CXCR1 | High affinity interleukin-8 receptor A (IL-8R A) (CXCR-1) (IL-8 receptor type 1) |
| CDw128b | CXCR2_HUMAN | P25025 | 146928 | IL8RB; CXCR2 | High affinity interleukin-8 receptor B (IL-8R B) (CXCR-2) (IL-8 receptor type 2) |
| CD129 | IL9R_HUMAN | Q01113 | 300007 | IL9R | Interleukin-9 receptor (IL-9R) |
| CD130 | IL6RB_HUMAN | P40189 | 600694 | IL6ST | Interleukin-6 receptor beta chain (IL-6R-beta) (interleukin 6 signal transducer) (Membrane glycoprotein 130) |
| | | | | | (Gp130) (Oncostatin M receptor) |
| CD131 | IL3RB_HUMAN | P32927 | 138981 | CSF2RB; IL5RN | Cytokine receptor common beta chain |
| CD132 | IL2RG_HUMAN | P31785 | 300400 | IL2RG | Cytokine receptor common gamma chain (Interleukin-2 receptor gamma chain) (IL-2R gamma chain) |
| CD133 | PROM1_HUMAN | O43490 | 604365 | PROM1; PROML1 | Prominin 1 (Prominin-like protein 1) (Antigen AC133) |
| CD134 | TNR4_HUMAN | P43489 | 600315 | TNFRSF4; TXGP1L | Tumor necrosis factor receptor superfamily member 4 (OX40L receptor) (ACT35 antigen) (TAX-transcriptionally |
| | | | | | activated glycoprotein 1 receptor) |
| CD135 | FLT3_HUMAN | P36888 | 136351 | FLT3; STK1 | FL cytokine receptor (EC 2.7.1.112) (Tyrosine-protein kinase receptor flt3) (Stem cell tyrosine kinase 1) |
| | | | | | (STK-1) |
| CDw136 | RON_HUMAN | Q04912 | 600168 | MST1R; RON | Macrophage-stimulating protein receptor (EC 2.7.1.112) (MSP receptor) (p185-RON) |
| CDw137 | TNR9_HUMAN | Q07011 | 602250 | TNFRSF9; ILA | Tumor necrosis factor receptor superfamily member 9 (4-1BB ligand receptor) (T-cell antigen 4-1BB) (T-cell |
| | | | | | antigen ILA) |
| CD138 | SDC1_HUMAN | P18827 | 186355 | SDC1; SDC | Syndecan-1 (SYND1) |
| CD139 | N.A. | N.A. | N.A. | N.A. | B-031 |
| CD140a | PGFRA_HUMAN | P16234 | 173490 | PDGFRA | Alpha platelet-derived growth factor receptor (EC 2.7.1.112) (PDGF-R-alpha) |
| CD140b | PGFRB_HUMAN | P09619 | 173410 | PDGFRB | Beta platelet-derived growth factor receptor (EC 2.7.1.112) (PDGF-R-beta) |
| CD141 | TRBM_HUMAN | P07204 | 188040 | THBD; THRM | Thrombomodulin (fetomodulin) (Tm) |
| CD142 | TF_HUMAN | P13726 | 134390 | F3 | Tissue factor (TF) (Coagulation factor III) (Thromboplastin) |
| CD143 | ACE_HUMAN | P12821 | 106180 | DCP1; ACE | Angiotensin-converting enzyme (EC 3.4.15.1) (ACE) (Dipeptidyl carboxypeptidase I) (Kininase II) |
| CD144 | CADH5_HUMAN | P33151 | 601120 | CDH5 | Vascular endothelial-cadherin (VE-cadherin) (Cadherin-5) (7B4 antigen) |
| CDw145 | N.A. | N.A. | N.A. | N.A. | N.A. |
| CD146 | MUC18_HUMAN | P43121 | 155735 | MCAM; MUC18 | Cell surface glycoprotein MUC18 (Melanoma-associated antigen MUC8) (Melanoma-associated antigen A32) (S-endo 1 |
| | | | | | endothelial-associated antigen) |
| CD147 | BASI_HUMAN | P35613 | 109480 | BSG | Basigin (Leukocyte activation antigen M6) (Collagenase stimulatory factor) (EMMPRIN) (5f7) |
| CD148 | PTPRJ_HUMAN | Q12913 | 600925 | PTPRJ; DEP1 | Protein-tyrosine phosphatase eta (EC 3.1.3.48) (R-PTP-eta) (Density enhanced phosphatase-1) (DEP-1) |
| CDw149 | N.A. | N.A. | N.A. | N.A. | [Deleted] |
| CD150 | SLAF1_HUMAN | Q13291 | 603492 | SLAMF1; SLAM | Signaling lymphocytic activation molecule (IPO-3) |
| CD151 | CD151_HUMAN | P48509 | 602243 | CD151 | Platelet-endothelial tetraspan antigen 3 (PETA-3) (Gp27) (Membrane glycoprotein SFA-1) |
| CD152 | CTLA4_HUMAN | P16410 | 123890 | CTLA4 | Cytotoxic T-lymphocyte protein 4 (CTLA-4) |
| CD153 | TNFL8_HUMAN | P32971 | 603875 | TNFSF8; CD30L | Tumor necrosis factor ligand superfamily member 8 (CD30 ligand) (CD30-L) |
| CD154 | TNFL5_HUMAN | P29965 | 308230 | TNFSF5; CD40L | Tumor necrosis factor ligand superfamily member 5 (CD40 ligand) (CD40-L) (TNF-related activation protein) |
| | | | | | (TRAP) (T cell antigen Gp39) |
| CD155 | PVR_HUMAN | P15151 | 173850 | PVR; PVS | Potiovirus receptor |
| CD156a | ADAM8_HUMAN | P78325 | 602267 | ADAM8; MS2 | ADAM 8 (EC 3.4.24.—) (A disintegrin and metalloproteinase domain 8) (Cell surface antigen MS2) |
| CD156b | ADA17_HUMAN | P78536 | 603639 | ADAM17; TACE | ADAM 17 (EC 3.4.24.—) (A disintegrin and metalloproteinase domain 17) (TNF-alpha converting enzyme) (TNF-alpha |
| | | | | | convertase) |
| CD157 | BST1_HUMAN | Q10588 | 600387 | BST1 | ADP-ribosyl cyclase 2 (EC 3.2.2.5) (Cyclic ADP-ribose hydrolase 2) (Bone marrow stromal antigen 1) (BST-1) |
| CD158a | KI2L1_HUMAN | P43626 | 604936 | KIR2DL1; NKAT1 | Killer cell immunoglobulin-like receptor 2DL1 (p58.1 MHC class-I specific NK receptor) (Natural killer |
| | | | | | associated transcript 1) (NKAT-1) (p58 natural killer cell receptor clones CL-42/47.11) (p58 NK receptor) (p58.1 MHC class-I specific NK |
| | | | | | receptor) |
| CD158b | KI2L3_HUMAN | P43628 | 604938 | KIR2DL3; NKAT3 | Killer cell immunoglobulin-like receptor 2DL3 (p58.2 MHC class-I specific NK receptor) |
| CD158d | KI2L4_HUMAN | Q99706 | 604945 | KIR2DL4 | Killer cell immunoglobulin-like receptor 2DL4 (MHC class I NK cell receptor KIR103AS) (Killer cell inhibitory |
| | | | | | receptor 103AS) (KIR-103AS) (G9P) |
| CD158g | KI2S5_HUMAN | Q14953 | 604956 | KIR2DS5; NKAT9 | Killer cell immunoglobulin-like receptor 2DS5 (MHC class I NK cell receptor) (Natural killer associated |
| | | | | | transcript 9) (NKAT-9) |
| CD158h | KI2S1_HUMAN | Q14954 | 604952 | KIR2DS1 | Killer cell immunoglobulin-like receptor 2DS1 (MHC class I NK cell receptor Eb6 Actl) |
| CD158i | KI2S4_HUMAN | P43632 | 604955 | KIR2DS4; NKAT8 | Killer cell immunoglobulin-like receptor 2DS4 (Natural killer associated transcript 8) (NKAT-8) (p58 natural |
| | | | | | killer cell receptor clone CL-39) (p58 NK receptor) (CL-17) |
| CD158j | KI2S2_HUMAN | P43631 | 604953 | KIR2DS2; NKAT5 | Killer cell immunoglobulin-like receptor 2DS2 (MHC class I NK cell receptor) (Natural killer associated |
| | | | | | transcript 5) (NKAT-5) (p58 natural killer cell receptor clone CL-49) (p58 NK receptor) (NK receptor 183 Actl) |
| CD158k | KI3L2_HUMAN | P43630 | 604947 | KIR3DL2; NKAT4 | Killer cell immunoglobulin-like receptor 3DL2 (MHC class I NK cell receptor) (Natural killer associated |
| | | | | | transcript 4) (NKAT-4) (p70 natural killer cell receptor clone CL-5) |
| CD158z | KI3L3_HUMAN | Q8N743 | N.A. | KIR3DL3 | Killer cell immunoglobulin-like receptor 3DL3. |
| CD159 | N.A. | N.A. | N.A. | N.A. | Not yet assigned |
| CD160 | BY55_HUMAN | O95971 | 604463 | CD160; BY55 | Natural killer cell receptor BY55 |
| CD161 | N.A. | N.A. | N.A. | N.A. | NKRP-1 |
| CD162 | SELPL_HUMAN | Q14242 | 600738 | SELPLG | P-selectin glycoprotein ligand 1 (PSGL-1) (Selectin P ligand) |
| CD163 | N.A. | N.A. | N.A. | N.A. | GHI/61 (M130) |
| CD164 | MUC24_HUMAN | Q04900 | 603356 | CD164 | Putative mucin core protein 24 (Multi-glycosylated core protein 24) (MGC-24) (Muc-24) |
| CD165 | N.A. | N.A. | N.A. | N.A. | AD2 (Gp37) |
| CD166 | CD166_HUMAN | Q13740 | 601662 | ALCAM; MEMD | Activated leukocyte-cell adhesion molecule |
| CD167a | DDR1_HUMAN | Q08345 | 600408 | DDR1; EDDR1; | CAK Epithelial discoidin domain receptor 1 (EC 2.7.1.112) (Tyrosine-protein kinase CAK) (Cell adhesion kinase) |
| | | | | | (Tyrosine kinase DDR) (Discoidin receptor tyrosine kinase) (TRK E) |
| CD168 | HMMR_HUMAN | O75330 | 600936 | HMMR; RHAMM | Hyaluronan mediated motility receptor (Intracellular hyaluronic acid binding protein) (Receptor for |
| | | | | | hyaluronan-mediated motility) |
| CD169 | SN_HUMAN | Q98ZZ2 | 600751 | SN | Sialoadhesin (Sialic acid binding Ig-like lectin-1) (Sigleo-1) |
| CD170 | SIGL5_HUMAN | O15389 | 604200 | SIGLEC5; OBBP2 | Sialic acid binding Ig-like lectin 5 (Siglec-5) (Obesity-binding protein 2) (OB binding protein-2) (OB-BP2) |
| | | | | | (CD33 antigen-like 2) |
| CD171 | L1CAM_HUMAN | P32004 | 308840 | L1CAM; CAML1 | Neural cell adhesion molecule L1 (N-CAM L1) |
| CD173 | N.A. | N.A. | N.A. | N.A | [Not a protein: Blood group H type 2] Carbohydrate |
| CD174 | N.A. | N.A. | N.A. | N.A | [Not a protein: Lewis y] Carbohydrate |
| CD175 | N.A. | N.A. | N.A. | N.A | [Not a protein: Tn] Carbohydrate |
| CD175s | N.A. | N.A. | N.A. | N.A | [Not a protein: Sialyl-Tn] Carbohydrate |
| CD176 | N.A. | N.A. | N.A. | N.A | [Not a protein: TF] Carbohydrate |
| CD178 | TNFL6_HUMAN | P48023 | 134638 | TNFSF6; APT1LG1 | Tumor necrosis factor ligand superfamily member 6 (FAS antigen ligand) (Apoptosis antigen ligand) (APTL) |
| CD179a | VPREB_HUMAN | P12018 | 605141 | VPREB1; VPREB | Immunoglobulin iota chain (V(pre)B protein) (VpreB protein) |
| CD179b | IGLL1_HUMAN | P15814 | 146770 | IGLL1; IGL1 | Immunoglobulin lambda-like polypeptide 1 (immunoglobulin-related 14.1 protein) (immunoglobulin omega |
| | | | | | polypeptide) (Lambda 5) |
| CD180 | CD180_HUMAN | Q99467 | 602226 | CD180; LY64 | CD180 antigen (Lymphocyte antigen 64) (Radioprotective 105 kDa protein) |
| CD183 | CXCR3_HUMAN | P49682 | 600894 | CXCR3; GPR9 | C-X-C chemokine receptor type 3 (CXC-R3) (CXCR-3) (CKR-L2) |
| CD184 | CXCR4_HUMAN | P61073 | 162643 | CXCR4 | C-X-C chemokine receptor type 4 (CXC-R4) (CXCR-4) (Stromal cell-derived factor 1 receptor) (SDF-1 receptor) |
| | | | | | (Fusin) (Leukocyte-derived seven transmembrane domain receptor) (LESTR) (LCR1) (FB22) |
| CD195 | CCR5_HUMAN | P51681 | 601373 | CCR5; CMKBR5 | C-C chemokine receptor type 5 (C-C CKR-5) (CC-CKR-5) (CCR-5) (CCR5) (HIV-1 fusion co-receptor) (CHEMR13) |
| CD200 | OX2G_HUMAN | P41217 | 155970 | CD200; MOX2 | OX-2 membrane glycoprotein precursor (My033 protein) |
| CD201 | EPCR_HUMAN | Q9UNN8 | 600646 | PROCR; EPCR | Endothelial protein C receptor (Endothelial cell protein C receptor) (Activated protein C receptor) (APC |
| | | | | | receptor) |
| CD202b | TIE2_HUMAN | Q02763 | 600221 | TEK; TIE2 | Angiopoietin 1 receptor (EC 2.7.1.112) (Tyrosine-protein kinase receptor TIE-2) (Tyrosine-protein kinase |
| | | | | | receptor TEK) (P140 TEK) (Tunica interna endothelial cell kinase) |
| CD203c | ENPP3_HUMAN | Q14638 | 602182 | ENNP3; PDNP3 | Ectonucleotide pyrophosphatase/phosphodiesterase 3 (E-NPP 3) (Phosphodiesterase I/nucleotide pyrophosphatase |
| | | | | | 3) (Phosphodiesterase I beta) (PD-Ibeta) |
| CD204 | MSRE_HUMAN | P21757 | 153622 | MSR1 | Macrophage scavenger receptor types I and II (Macrophage acetylated LDL receptor I and II) |
| CD205 | LY75_HUMAN | Q60449 | 604524 | LY75; CD205 | Lymphocyte antigen 75 (DEC-205) (gp200-MR6) |
| CD206 | MANR1_HUMAN | P22897 | 153618 | MRG1 | Macrophage mannose receptor (MMR) |
| CD209 | CD209_HUMAN | Q9NNX6 | 604672 | CD209 | CD209 antigen (Dendritic cell-specific ICAM-3-grabbing nonintegrin 1) (DC-SIGN1) (DC-SIGN) |
| CD213a1 | I13R1_HUMAN | P78552 | 300119 | IL13RA1; IL13RA | Interleukin-13 receptor alpha-1 chain (IL-13R-alpha-1) (IL-13RA-1) |
| CD220 | INSR_HUMAN | P06213 | 147670 | INSR | Insulin receptor (EC 2.7.1.112) (IR) |
| CD221 | IGF1R_HUMAN | P08069 | 147370 | IGF1R | Insulin-like growth factor I receptor (EC 2.7.1.112) |
| CD222 | MPRI_HUMAN | P11717 | 147280 | IGF2R; MPRI | Cation-independent mannose-6-phosphate receptor (CI Man-6-P receptor) (CI-MPR) (Insulin-like growth factor II |
| | | | | | receptor) (300 kDa mannose 6-phosphate receptor) (MPR 300) (MPR300) |
| CD223 | LAG3_HUMAN | P18627 | 153337 | LAG3; FDC | Lymphocyte activation gene-3 protein (LAG-3) (FDC protein) |
| CD224 | GGT1_HUMAN | P19440 | 231950 | GGT1; GGT | Gamma-glutamyltranspeptidase 1 (EC 2.3.2.2) (Gamma-glutamyltransferase 1) |
| CD225 | IFM1_HUMAN | P13164 | 604456 | IFITM1; IFI17 | Interferon-induced transmembrane protein 1 (Interferon-induced protein 17) (Interferon-inducible protein 9-27) |
| | | | | | (Leu-13 antigen) |
| CD226 | CD226_HUMAN | Q15762 | 605397 | CD226; DNAM1 | CD226 antigen (DNAX accessory molecule-1) (DNAM-1) |
| CD227 | MUC1_HUMAN | P15941 | 158340 | MUC1 | Mucin 1 (MUC-1) (Polymorphic epithelial mucin) (PEM) (PEMT) (Episialin) (Tumor-associated mucin) (Carcinoma- |
| | | | | | associated mucin) |
| CD228 | TRFM_HUMAN | P08582 | 155750 | MFI2; MAP97 | Melanotransferrin (Melanoma-associated antigen p97) |
| CD229 | LY9_HUMAN | Q9HBG7 | 600684 | LY9 | T-lymphocyte surface antigen Ly-9 (Lymphocyte antigen 9) (Cell-surface molecule Ly-9) |
| CD230 | PRIO_HUMAN | P04156 | 176640 | PRNP | Major prion protein (PrP) (PrP27-30) (PrP33-35C) (ASCR) |
| CD231 | TSN7_HUMAN | P41732 | 300096 | TPSAN7; TM4SF2 | Tetraspanin-7 (Tspan7) (Transmembrane 4 superfamily, member 2) (Cell surface glycoprotein A15) (T-cell acute |
| | | | | | lymphoblastic leukemia associated antigen 1) (TALLA-1) (Membrane component, X chromosome, surface marker 1) |
| CD233 | B3AT_HUMAN | P02730 | 109270 | SLC4A1; AE1 | Band 3 anion transport protein (Anion exchange protein 1) (AE 1) |
| CD234 | DUFFY_HUMAN | Q16570 | 110700 | FY; GPD; DARC | Duffy antigen (Fy glycoprotein) (Glycoprotein D) (GpFy) |
| CD235a | GLPA_HUMAN | P02724 | 111300 | GYPA; GPA | Glycophorin A (PAS-2) (Sialoglycoprotein alpha) (MN sialoglycoprotein) |
| CD235b | GLPB_HUMAN | P06028 | 111740 | GYPB; GPB | Glycophorin B (PAS-3) (Sialoglycoprotein delta) (SS-active sialoglycoprotein) |
| CD238 | KELL_HUMAN | P23276 | 110900 | KEL | Kell blood group glycoprotein (EC 3.4.24.—) |
| CD242 | ICAM4_HUMAN | Q14773 | 111250 | ICAM4; LW | Intercellular adhesion molecule-4 (ICAM-4) (Landsteiner-Wiener blood group glycoprotein) (LW blood group |
| | | | | | protein) |
| CD243 | MDR1_HUMAN | P08183 | 171050 | ABCB1; PGY1 | Multidrug resistance protein 1 (P-glycoprotein 1) |
| CD244 | CD244_HUMAN | Q9BZW8 | 605554 | CD244; 284 | Natural killer cell receptor 284 (NKR284) (NK cell type I receptor protein 2B4) (h2B4) (NK cell activation |
| | | | | | inducing ligand) (NAIL) |
| CD246 | ALK_HUMAN | Q9UM73 | 105590 | ALK | ALK tyrosine kinase receptor (EC 2.7.1.112) (Anaplastic lymphoma kinase) |
| CD273 | PDL2_HUMAN | Q9B051 | 605723 | PDCD1LG2; B7DC | Programmed cell death 1 ligand 2 (Programmed death ligand 2) (PD-L2) (PD-1-ligand 2) (PDCD1 ligand 2) |
| | | | | | (Butyrophilin B7-DC) (B7-DC) |
| CD274 | PDL1_HUMAN | Q9NZQ7 | 605402 | CD274; PDCD1LG1 | Programmed cell death 1 ligand 1 (Programmed death ligand 1) (PD-L1) (PDCD1 ligand 1) (B7-homolog 1) (B7-H1) |
| CD278 | ICOS_HUMAN | Q9Y6W8 | 604558 | ICOS; AILIM | Inducible T-cell co-stimulator (Activation-inducible lymphocyte immunomediatory molecule) |
| CD281 | TLR1_HUMAN | Q15399 | 601194 | TLR1 | Toll-like receptor 1 (Toll/interleukin-1 receptor-like) (TIL) |
| CD282 | TLR2_HUMAN | O60603 | 603028 | TLR2; TIL4 | Toll-like receptor 2 (Toll/interleukin-1 receptor-like protein 4) |
| CD283 | TLR3_HUMAN | O15455 | 603029 | TLR3 | Toll-like receptor 3 |
| CD284 | TLR4_HUMAN | O00206 | 603030 | TLR4 | Toll-like receptor 4 |
| CD289 | TLR9_HUMAN | Q9NR96 | 605474 | TLR9 | Toll-like receptor 9 |

As used herein the term “differentiation/maturation stage” refers, when it refers to a cell lineage, to stages or levels in which the lineage is differentiated from a stem cell corresponding thereto to a differentiated cell. Conventionally, such a differentiation level has not been or could not be measured. However the present invention enables identification of such differentiation/maturation stages. Therefore, it is possible to determine whether a cell of interest follows a specific differentiation pathway, and thus tailor-made therapy can be provided.

(Classification of Stages)

The present invention allows classification of a variety of stages of a (stem) cell, according to the following markers. The more of such markers are used, the more accurate the classification of stages can be made. Accordingly, preferably, all the CD markers may be used, however the present invention is not limited thereto.

As used herein the monocytic lineage may be classified into Stage 1, Stage 2 and Stage 3.

Monocytic lineage stage 1 possesses the feature in which HLA-DR is positive, CD11b is negative, CD45 is weak positive, CD10 is negative, CD16 is negative, CD13 is weak positive, CD14 is negative. The following pattern may be used for identification of cells in monocytic lineage stage 1: FSC high level SSC intermediate, and FSC high level HLA-DR positive region, and FSC high level CD11b negative region and FSC high level CD45 weak positive region and FSC high level CD10 negative region and FSC high level CD16 negative region and FSC high level CD13 weak positive region and FSC high level CD14 negative region and SSC intermediate HLA-DR positive region and SSC intermediate CD11b negative region and SSC intermediate CD45 weak positive region and SSC intermediate CD10 negative region and ssc intermediate CD16 negative region and SSC intermediate CD13 weak positive region and SSC intermediate CD14 negative region and CD45 weak positive region HLA-DR positive region and CD45 weak positive region CD11b negative region and CD45 weak positive region CD10 negative region and CD45 weak positive region CD16 negative region and CD45 weak positive region CD13 weak positive region and CD45 weak positive region CD14 negative region and HLA-DR positive CD11b negative region and CD10 negative CD11b negative region and CD10 negative HLA-DR positive region and CD16 negative CD13 weak positive region and CD11b negative CD13 weak positive region and CD11b negative CD16 negative region and CD16 negative CD14 negative region and CD10 negative CD14 negative region and CD10 negative CD16 negative region (see FIG. 1B).

Monocytic lineage stage 2 possesses the feature in which HLA-DR is positive, CD11b is positive, CD45 is positive, CD10 is negative, CD16 is negative, CD13 is positive, CD14 is weak positive. The following patterns may be used for identification of cells in monocytic lineage stage 2: FSC high level SSC intermediate, and FSC high level HLA-DR positive region and FSC high level CD11b positive region and FSC high level CD45 positive region and FSC high level CD10 negative region and FSC high level CD16 negative region and FSC high level CD13 positive region and FSC high level CD14 weak positive region and SSC intermediate HLA-DR positive region and SSC intermediate CD11b positive region and SSC intermediate CD45 positive region and SSC intermediate CD10 negative region and SSC intermediate CD16 negative region and SSC intermediate CD13 positive region and SSC intermediate CD14 weak positive region and CD45 positive region HLA-DR positive region and CD45 positive region CD11b positive region and CD45 positive region CD10 negative region and CD45 positive region CD16 negative region and CD45 positive region CD13 positive region and CD45 positive region CD14 weak positive region and HLA-DR positive CD11b positive region and CD10 negative CD11b positive region and CD10 negative HLA-DR positive region and CD16 negative CD13 positive region and CD11b positive CD13 positive region and CD11b positive CD16 negative region and CD16 negative CD14 weak positive region and CD10 negative CD14 weak positive region and CD10 negative CD16 negative region (see FIG. 1C).

Monocytic lineage stage 3 possesses the feature in which HLA-DR is positive, CD11b is positive, CD45 is positive, CD10 is negative, CD16 is negative to weak positive, CD13 is positive, CD14 is positive. The following patterns may be used for identification of cells in monocytic lineage stage 3: FSC high level SSC intermediate, and FSC high level HLA-DR positive region and FSC high level CD11b positive region and FSC high level CD45 positive region and FSC high level CD10 negative region and FSC high level CD16 negative to weak positive region and FSC high level CD13 positive region and FSC high level CD14 positive region and SSC intermediate HLA-DR positive region and SSC intermediate CD11b positive region and SSC intermediate CD45 positive region and SSC intermediate CD10 negative region and SSC intermediate CD16 negative to weak positive region and SSC intermediate CD13 positive region and SSC intermediate CD14 positive region and CD45 positive HLA-DR positive region and CD45 positive CD11b positive region and CD45 positive CD10 negative region and CD45 positive CD16 negative to weak positive region and CD45 positive CD13 positive region and CD45 positive CD14 positive region and HLA-DR positive CD11b positive region and CD10 negative CD11b positive region and CD10 negative HLA-DR positive region and CD16 negative to weak positive CD13 positive region and CD11b positive CD13 positive region and CD11b positive CD16 negative to weak positive region and CD16 negative to weak positive CD14 positive region and CD10 negative CD14 positive region and CD10 negative CD16 negative to weak positive region (see FIG. 1D).

Myeloid lineage Stage 1 possesses the characteristic in which HLA-DR is weak positive to positive, CD11b is negative, CD45 is weak positive, CD10 is negative, CD16 is negative to weak positive, CD13 is weak positive, CD14 is negative. The following patterns may be used for identification of cells in myeloid lineage Stage 1: FSC high level SSC high level, and FSC high level HLA-DR weak positive to positive region and FSC high level CD11b negative region and FSC high level CD45 weak positive region and FSC high level CD10 negative region and FSC high level CD16 negative to weak positive region and FSC high level CD13 weak positive region and FSC high level CD14 negative region and SSC high level HLA-DR weak positive to positive region and SSC high level CD11b negative region and SSC high level CD45 weak positive region and SSC high level CD10 negative region and SSC high level CD16 negative to weak positive region and SSC high level CD13 weak positive region and SSC high level CD14 negative region and CD45 weak positive HLA-DR weak positive to positive region and CD45 weak positive CD11b negative region and CD45 weak positive CD10 negative region and CD45 weak positive CD16 negative to weak positive region and CD45 weak positive CD13 weak positive region and CD45 weak positive CD14 negative region and HLA-DR weak positive to positive CD11b negative region and CD10 negative CD11b negative region and CD10 negative HLA-DR weak positive to positive region and CD16 negative to weak positive CD13 weak positive region and CD11b negative CD13 weak positive region and CD11b negative CD16 negative to weak positive region and CD16 negative to weak positive CD14 negative region and CD10 negative CD14 negative region and CD10 negative CD16 negative to weak positive region (see FIG. 1E).

Myeloid lineage Stage 2 possesses the characteristic in which HLA-DR is negative to weak positive, CD11b is weak positive, CD45 is weak positive, CD10 is negative, CD16 is weak positive, CD13 is weak positive, CD14 is negative. The following patterns may be used for identification of cells in myeloid lineage Stage 2: FSC high level SSC high level, and FSC high level HLA-DR negative to weak positive region and FSC high level CD11b weak positive region and FSC high level CD45 weak positive region and FSC high level CD10 negative region and FSC high level CD16 weak positive region and FSC high level CD13 weak positive region and FSC high level CD14 negative region and SSC high level HLA-DR negative to weak positive region and SSC high level CD11b weak positive region and SSC high level CD45 weak positive region and SSC high level CD10 negative region and SSC high level CD16 weak positive region and SSC high level CD13 weak positive region and SSC high level CD14 negative region and CD45 weak positive HLA-DR negative to weak positive region and CD45 weak positive CD11b weak positive region and CD45 weak positive CD10 negative region and CD45 weak positive CD16 weak positive region and CD45 weak positive CD13 weak positive region and CD45 weak positive CD14 negative region and HLA-DR negative to weak positive CD11b weak positive region and CD10 negative CD11b weak positive region and CD10 negative HLA-DR negative to weak positive region and CD16 weak positive CD13 weak positive region and CD11b weak positive CD13 weak positive region and CD11b weak positive CD16 weak positive region and CD16 weak positive CD14 negative region and CD10 negative CD14 negative region and CD10 negative CD16 weak positive region (see FIG. 1F).

Myeloid lineage Stage 3 possesses characters in which HLA-DR is negative to weak positive, CD11b is positive, CD45 is weak positive to positive, CD10 is negative, CD16 is weak positive, CD13 is weak positive, CD14 is negative. The following patterns may be used for identification of cells in myeloid lineage Stage 3: FSC high level SSC high level, and FSC high level HLA-DR negative to weak positive region and FSC high level CD11b positive region and FSC high level CD45 weak positive to positive region and FSC high level CD10 negative region and FSC high level CD16 weak positive region and FSC high level CD13 weak positive region and FSC high level CD14 negative region and SSC high level HLA-DR negative to weak positive region and SSC high level CD11b positive region and SSC high level CD45 weak positive to positive region and SSC high level CD10 negative region and SSC high level CD16 weak positive region and SSC high level CD13 weak positive region and SSC high level CD14 negative region and CD45 weak positive to positive HLA-DR negative to weak positive region and CD45 weak positive to positive CD11b positive region and CD45 weak positive to positive CD10 negative region and CD45 weak positive to positive CD16 weak positive region and CD45 weak positive to positive CD13 weak positive region and CD45 weak positive to positive CD14 negative region and HLA-DR negative to weak positive CD11b positive region and CD10 negative CD11b positive region and CD10 negative HLA-DR negative to weak positive region and CD16 negative CD13 weak positive region and CD11b positive CD13 weak positive region and CD11b positive CD16 negative region and CD16 negative CD14 negative region and CD10 negative CD14 negative region and CD10 negative CD16 negative region (see FIG. 1G).

Myeloid lineage Stage 4 possesses the characteristic in which HLA-DR is negative to weak positive, CD11b is positive, CD45 is weak positive to positive, CD10 is weak positive, CD16 is positive, CD13 is positive, CD14 is negative. The following patterns may be used for identification of cells in myeloid lineage Stage 4: FSC high level SSC high level, and FSC high level HLA-DR negative to weak positive region and FSC high level CD11b positive region and FSC high level CD45 weak positive to positive region and FSC high level CD10 weak positive region and FSC high level CD16 positive region and FSC high level CD13 positive region and FSC high level CD14 negative region and SSC high level HLA-DR negative to weak positive region and SSC high level CD11b positive region and SSC high level CD45 weak positive to positive region and SSC high level CD10 weak positive region and SSC high level CD16 positive region and SSC high level CD13 positive region and SSC high level CD14 negative region and CD45 weak positive to positive HLA-DR negative to weak positive region and CD45 weak positive to positive CD11b positive region and CD45 weak positive to positive CD10 weak positive region and CD45 weak positive to positive CD16 positive region and CD45 weak positive to positive CD13 positive region and CD45 weak positive to positive CD14 negative region and HLA-DR negative to weak positive CD11b positive region and CD10 weak positive CD11b positive region and CD10 weak positive HLA-DR negative to weak positive region and CD16 positive CD13 positive region and CD11b positive CD13 positive region and CD11b positive CD16 positive region and CD16 positive CD14 negative region and CD10 weak positive CD14 negative region and CD10 weak positive CD16 positive region (see FIG. 1H).

Myeloid lineage Stage 5 possesses the characteristic in which HLA-DR is negative to weak positive, CD11b is positive, CD45 is positive, CD10 is positive, CD16 is positive, CD13 is positive, CD14 is negative. The following patterns may be used for identification of cells in myeloid lineage Stage 5: FSC high level SSC high level, and FSC high level HLA-DR negative to weak positive region and FSC high level CD11b positive region and FSC high level CD45 positive region and FSC high level CD10 positive region and FSC high level CD16 positive region and FSC high level CD13 positive region and FSC high level CD14 negative region and SSC high level HLA-DR negative to weak positive region and SSC high level CD11b positive region and SSC high level CD45 positive region and SSC high level CD10 positive region and SSC high level CD16 positive region and SSC high level CD13 positive region and SSC high level CD14 negative region and CD45 positive region HLA-DR negative to weak positive region and CD45 positive region CD11b positive region and CD45 positive region CD10 positive region and CD45 positive region CD16 positive region and CD45 positive region CD13 positive region and CD45 positive region CD14 negative region and HLA-DR negative to weak positive CD11b positive region and CD10 positive CD11b positive region and CD10 positive HLA-DR negative to weak positive region and CD16 positive CD13 positive region and CD11b positive CD13 positive region and CD11b positive CD16 positive region and CD16 positive CD14 negative region and CD10 positive CD14 negative region and CD10 positive CD16 positive region (see FIG. 1I).

B lymphocytic lineage Stage 1 possesses characters in which CD20 is negative, CD10 is positive, CD45 is weak positive, CD34 is positive, CD5 is negative, CD19 is weak positive to positive. The following patterns may be used for identification of cells in B lymphocytic lineage Stage 1: FSC low level SSC low level and FSC low level CD20 negative region and FSC low level CD10 positive region and FSC low level CD45 weak positive region and FSC low level CD34 positive region and FSC low level CD5 negative region and FSC low level CD19 weak positive to weak positive region and SSC low level CD20 negative region and SSC low level CD10 positive region and SSC low level CD45 weak positive region and SSC low level CD34 positive region and SSC low level CD5 negative region and SSC low level CD19 weak positive to positive region and CD45 weak positive CD20 negative region and CD45 weak positive CD10 positive region and CD45 weak positive CD34 positive region and CD45 weak positive CD5 negative region and CD45 weak positive CD19 weak positive to positive region and CD20 negative CD10 positive region and CD34 positive CD10 positive region and CD34 positive CD20 negative region and CD5 negative CD19 weak positive to positive region and CD34 positive CD19 weak positive to positive region, CD34 positive CD5 negative region (see FIG. 1J).

B lymphocytic lineage Stage 2 possesses the characteristic in which CD20 is weak positive, CD10 is positive, CD45 is weak positive to positive, CD34 is negative, CD5 is negative, CD19 is weak positive to positive. The following patterns may be used for identification of cells in B lymphocytic lineage Stage 2: FSC low level SSC low level and FSC low level CD20 weak positive region and FSC low level CD10 positive region and FSC low level CD45 weak positive to positive region and FSC low level CD34 negative region and FSC low level CD5 negative region and FSC low level CD19 weak positive to positive region and SSC low level CD20 weak positive region and SSC low level CD10 positive region and SSC low level CD45 weak positive to positive region and SSC low level CD34 negative region and SSC low level CD5 negative region and SSC low level CD19 weak positive to positive region and CD45 weak positive to positive CD20 weak positive region and CD45 weak positive to positive CD10 positive region and CD45 weak positive to positive CD34 negative region and CD45 weak positive to positive CD5 negative region and CD45 weak positive to positive CD19 weak positive to positive region and CD20 weak positive CD10 positive region and CD34 negative CD10 positive region and CD34 negative CD20 weak positive region and CD5 negative CD19 weak positive to positive region and CD34 negative CD19 weak positive to positive region, CD34 negative CD5 negative region (see FIG. 1K).

B lymphocytic lineage Stage 3 possesses the characteristic in which CD20 is positive, CD10 is negative to weak positive, CD45 is positive, CD34 is negative, CD5 is weak positive, CD19 is positive. The following patterns may be used for identification of cells in B lymphocytic lineage Stage 3: FSC low level SSC low level and FSC low level CD20 positive region and FSC low level CD10 negative to weak positive region and FSC low level CD45 positive region and FSC low level CD34 negative region and FSC low level CD5 weak positive region and FSC low level CD19 positive region and SSC low level CD20 positive region and SSC low level CD10 negative to weak positive region and SSC low level CD45 positive region and SSC low level CD34 negative region and SSC low level CD5 weak positive region and SSC low level CD19 positive region and CD45 positive CD20 positive region and CD45 positive CD10 negative to weak positive region and CD45 positive CD34 negative region and CD45 positive CD5 weak positive region and CD45 positive CD19 positive region and CD20 positive CD10 negative to weak positive region and CD34 negative CD10 negative to weak positive region and CD34 negative CD20 positive region and CD5 weak positive CD19 positive region and CD34 negative CD19 positive region, CD34 negative CD5 weak positive region (see FIG. 1L).

B lymphocytic lineage Stage 4 possesses the characteristic in which CD20 is positive, CD10 is negative, CD45 is positive, CD34 is negative, CD5 is negative, CD19 is positive. The following patterns may be used for identification of cells in B lymphocytic lineage Stage 4: FSC low level SSC low level and FSC low level CD20 positive region and FSC low level CD10 negative region and FSC low level CD45 positive region and FSC low level CD34 negative region and FSC low level CD5 negative region and FSC low level CD19 positive region and SSC low level CD20 positive region and SSC low level CD10 negative region and SSC low level CD45 positive region and SSC low level CD34 negative region and SSC low level CD5 negative region and SSC low level CD19 positive region and CD45 positive CD20 positive region and CD45 positive CD10 negative region and CD45 positive CD34 negative region and CD45 positive CD5 negative region and CD45 positive CD19 positive region and CD20 positive CD10 negative region and CD34 negative CD10 negative region and CD34 negative CD20 positive region and CD5 negative CD19 positive region and CD34 negative CD19 positive region, CD34 negative CD5 negative region (see FIG. 1M).

T lymphocytic lineage Stage 1 possesses the characteristic in which CD1a is negative to weak positive, CD34 is negative to weak positive, CD45 is weak positive, CD3 is negative, CD8 is negative, CD10 is positive, CD4 is negative. The following patterns may be used for identification of cells in T lymphocytic lineage Stage 1: FSC low level SSC low level and FSC low level CD1a negative to weak positive region and FSC low level CD34 negative to weak positive region and FSC low level CD45 weak positive region and FSC low level CD3 negative region and FSC low level CD8 negative region and FSC low level CD10 strong positive region and FSC low level CD4 negative region and SSC low level CD1a negative to weak positive region and SSC low level CD34 negative to weak positive region and SSC low level CD45 weak positive region and SSC low level CD3 negative region and SSC low level CD8 negative region and SSC low level CD10 strong positive region and SSC low level CD4 negative region and CD45 weak positive CD1a negative to weak positive region and CD45 weak positive CD34 negative to weak positive region and CD45 weak positive CD3 negative region and CD45 weak positive CD8 negative region and CD45 weak positive CD10 strong positive region and CD45 weak positive CD4 negative region and CD1a negative to weak positive CD34 negative to weak positive region and CD3 negative CD34 negative to weak positive region and CD3 negative CD1a negative to weak positive region and CD8 negative CD10 strong positive region and CD4 negative CD10 strong positive region and CD4 negative CD8 negative region (see FIG. 1N).

T lymphocytic lineage Stage 2 possesses the characteristic in which CD1a is positive, CD34 is negative, CD45 is weak positive, CD3 is negative, CD8 is weak positive to positive, CD10 is weak positive to positive, CD4 is weak positive to positive. The following patterns may be used for identification of cells in T lymphocytic lineage Stage 2: FSC low level SSC low level and FSC low level CD1a positive region and FSC low level CD34 negative region and FSC low level CD45 weak positive region and FSC low level CD3 negative region and FSC low level CD8 weak positive to positive region and FSC low level CD10 weak positive to positive region and FSC low level CD4 weak positive to positive region and SSC low level CD1a positive region and SSC low level CD34 negative region and SSC low level CD45 weak positive region and SSC low level CD3 negative region and SSC low level CD8 weak positive to positive region and SSC low level CD10 weak positive to positive region and SSC low level CD4 weak positive to positive region and CD45 weak positive CD1a positive region and CD45 weak positive CD34 negative region and CD45 weak positive CD3 negative region and CD45 weak positive CD8 weak positive to positive region and CD45 weak positive CD10 weak positive to positive region and CD45 weak positive CD4 weak positive to positive region and CD1a positive CD34 negative region and CD3 negative CD34 negative region and CD3 negative CD1a positive region and CD8 weak positive to positive CD10 weak positive to positive region and CD4 weak positive to positive CD10 weak positive to positive region and CD4 weak positive to positive CD8 weak positive to positive region (see FIG. 1O).

T lymphocytic lineage Stage 3 possesses the characteristic in which CD1a is weak positive, CD34 is negative, CD45 is positive, CD3 is weak positive, CD8 is positive, CD10 is weak positive, CD4 is positive. The following patterns may be used for identification of cells in T lymphocytic lineage Stage 3: FSC low level SSC low level and FSC low level CD1a weak positive region and FSC low level CD34 negative region and FSC low level CD45 positive region and FSC low level CD3 weak positive region and FSC low level CD8 positive region and FSC low level CD10 weak positive region and FSC low level CD4 positive region and SSC low level CD1a weak positive region and SSC low level CD34 negative region and SSC low level CD45 positive region and SSC low level CD3 weak positive region and SSC low level CD8 positive region and SSC low level CD10 weak positive region and SSC low level CD4 positive region and CD45 positive CD1a weak positive region and CD45 positive CD34 negative region and CD45 positive CD3 weak positive region and CD45 positive CD8 positive region and CD45 positive CD10 weak positive region and CD45 positive CD4 positive region and CD1a weak positive CD34 negative region and CD3 weak positive CD34 negative region and CD3 weak positive CD1a weak positive region and CD8 positive CD10 weak positive region and CD4 positive CD10 weak positive region and CD4 positive CD8 positive region (see FIG. 1P).

T lymphocytic lineage Stage 4 possesses the characteristic in which CD1a is negative, CD34 is negative, CD45 is positive, CD3 is positive, CD10 is negative, CD4 and CD8 are positive, but no cell with positive for both CD4 and CD8. The following patterns may be used for identification of cells in T lymphocytic lineage Stage 4: FSC low level SSC low level and FSC low level CD1a negative region and FSC low level CD34 negative region and FSC low level CD45 positive region and FSC low level CD3 positive region and FSC low level CD10 negative region and SSC low level CD1a negative region and SSC low level CD34 negative region and SSC low level CD45 positive region and SSC low level CD3 positive region and SSC low level CD10 negative region and CD45 positive CD1a negative region and CD45 positive CD34 negative region and CD45 positive CD3 positive region and CD45 positive CD10 negative region and CD1a negative CD34 negative region and CD3 positive CD34 negative region and CD3 positive CD1a negative region and CD4 and CD8 are positive region, respectively (see FIG. 1Q).

A marker which may be used in the present invention includes, but is not limited to, for example: CD1a, CD2, CD3, CD4, CD5, CD7, CD8, CD10, CD11b, CD13, CD14, CD15, CD16, CD19, CD20, CD22, CD23, CD33, CD34, CD45, TdT, FMC7 and HLA-DR, and the like.

Cellular markers used in the present invention for B lymphocytic lineage, include, but are not limited to: the CD10, CD19, CD34 and CD45; the combination of CD10, CD20, CD5 and CD45; the combination of CD19, CD23, CD5 and CD45; the combination of CD10, CD20, CD34 and CD45; and the combination of CD5, CD19, CD34 and CD45, and the like.

Cellular markers used in the present invention for T lymphocytic lineage, include, but are not limited to: the combination of CD1a, CD3, CD10 and CD45; the combination of CD4, CD8, CD7 and CD45; the combination of CD1a, CD3, CD34 and CD45; and the combination of CD4, CD8, CD11 and CD45, and the like.

Cellular markers used in the present invention for myeloid lineage or monocytic lineage, include, but are not limited to: the combination of HLA-DR, CD11b, CD10 and CD45; the combination of CD16, CD13, CD33 and CD45; the combination of CD4, CD10, CD34 and CD45; the combination of CD11b, CD13, CD16 and CD45; and the combination of CD10, CD4, CD14 and CD45, and the like.

It should be understood that such cellular markers are measured with respect to their expression level to allow the identification of stages. These levels may be evaluated by absolute or relative levels.

(Screening)

As used herein, the term “screening” refers to selection of a target, such as an organism, a substance, or the like, a given specific property of interest from a population containing a number of elements using a specific operation/evaluation method. For screening, the method or system of the present invention can be used.

As used herein conducting screening using immunological reactions also refers to “immunophenotyping”. In this case, antibodies or single stranded antibodies used in the present invention may be used for immunophenotype classification of cell-lines and biological samples. Transcription products and translation products of the present invention may be useful as cellular markers which are differently expressed in a variety of stages of a specific cell type differentiation and/or maturation stages. Monoclonal antibodies directed to a specific epitope or a combination of epitopes, allow screening of cell populations expressing markers. A variety of technologies may be used to screen cell populations expressing markers using monoclonal antibodies, and such technologies include, but are not limited to, for example, magnetic separation using magnetic beads coated with an antibody, “panning” using an antibody attached to solid matrix (i.e. plate), and flow cytometry (see, for example, U.S. Pat. No. 5,985,660; and Morrison et al., Cell, 96: 737-49 (1999))

(Exemplary Separation of Human Peripheral Blood Leukocytes Using Flow Cytometry and Measurement of Lymphocyte Subset by Flow Cytometry)

As used herein the term “flow cytometry” refers to a technology for physically, chemically and biologically measuring individual cells, individual or other biological particles suspended in a liquid. Apparatus using this technology is referred to as a “flow cytometer”. Flow cytometers are an apparatus or system for measuring the optical properties of a floating material or cell in a homogenous suspension of cells. Cells are suspended in a flow of liquid, and pass through the focus of laser beam, at which point five different optical properties, forward scattered light, side scattered light, and three different wavelengths of fluorescence are measured simultaneously with respect to each cell amongst 500-4,000 cells. Thereafter, a number of biological properties of the cell, such as size, intracellular structure, a variety of antigens, nucleic acid amounts and the like, can be accurately measured.

Scattered light refers to light which is scattered to the peripheral area after the laser beam is focused onto a cell. Forward scatter (FSC) is used to detect the forward direction with respect to the laser light axis, and thus the scattered light intensity thereof is proportional to the surface area of the cell of interest. That is, if the value of FSC is relatively large, the size of such a cell is also large, and if the value of FSC thereof is small, then the size of such a cell is also small. Side scatter (SSC) is detected at a location of 90 degrees (vertical) to the laser light axis, and the scattered light intensity is proportional to the state of cellular particles or cellular structure. That is, if the value of SSC is relatively large, the intracellular structure of the cell is complex, and if the value of SSC is small, then the intracellular structure is simple.

Results of flow cytometry are typically represented by dot plot in which FSC is plotted on the x axis, and SSC is plotted on the y axis. Each cell is represented by a single dot (point) in a figure, and the location thereof is determined by a relative value of FSC and SSC. Lymphocytes with relatively small size and intracellular structure is simple, are located at lower left panel, and those (granulocytes) with relatively large size and granules therein are located at upper right panel, those (monocytes) with relatively large size and simple intracellular structure are located between lymphocytes and granulocytes, and those cells are separately represented as forming a group or population.

Fluorescence refers to a light emitted from a fluorophore used to label a cell upon excitation. Excitation is induced by radiating the fluorophore with a laser beam. Flow cytometers (for example, product name: Becton & Dickinson FACSCalibur) radiate single wavelength laser beams at 488 nm and at 635 nm. Cells per se also have the property of emitting weak fluorescence (autofluorescence), and in fact, when specific detecting molecules possessed by the cell are fluorescent, it is necessary to bind fluorescence to the cell or molecules possessed by the same, in any advanced manner. For example, FITC (Fluorescein isothiocyanate) absorbs excitation light at 488 nm, and emits fluorescence (green) mainly at 530 nm. When antibodies are labeled with FITC in advance, the total amount of antibody bound varies depending on the amount of antigen present on the surface of a cell. As a result, the fluorescence intensity of FITC will be different. Therefore, the amount of antigen present on the surface of the cell can be inferred therefrom. FACS Calibur, which can be used as an example, is equipped with four fluorescence detectors capable of detecting at different fluorescent wavelength regions, and thus allows the simultaneous detection of four different antigens at maximum, when a plurality of fluorescent dyes are prepared which emit lights at different wavelengths. Fluorescent dyes other than FITC excited by single wavelength laser light at 488 nm, include phycoerythrin (PE), which emits fluorescence mainly at 585 nm, peridinin chlorophyll protein (PerCP) and carybocyanin-5 (PE-Cy5) which emit mainly at 670 nm. Allophycocyanin (APC), which is a fluorescent dye excited by a single wavelength laser light at 635 nm, emits fluorescence at 670 nm. These fluorescent dyes are combined with a variety of antibodies for use in the double or triple staining of cells. CD19 molecules expressed on the surface of B lymphocytes, CD4 and CD8 molecules expressed on the surface of T lymphocytes, and the like, may be detected by the use of a monoclonal antibody that specifically reacting with them.

Strictly speaking, flow cytometry is an improved or developed technology from fluorescence-activated cell sorter (FACS). As used herein, the term “FACS” refers to a method and an apparatus in which a laser beam is used to analyze surface antigen on a free cell, such as lymphocytes, or to separate and analyze cells by means of the presence and absence of surface antigens.

The results of flow cytometry may be represented by a histogram, dot plot, or the like.

As used herein the term “histogram” refers to a graph in which the light intensity and cell number of each parameter is depicted as x-axis and y-axis, respectively, in a fluorescence measurement using a flow cytometer. Such a format allows more than 10,000 cells to be counted in total.

As used herein the term “dot plot” refers to a plot in which the fluorescence intensities of two fluorophores are plotted on x- and y-axes. When staining by two-color and three-color, each fluorescence intensity thereof is plotted onto the x- or y-axis, and each cell is represented so as to correspond each cell to a dot on the two-dimensional graph for analysis.

For example, after removing peripheral blood or bone marrow liquid, the sample is subjected to hemolysis or specific gravity centrifugation to remove erythrocytes, and reacted with fluorescent labeling antibodies (antibodies against the antigen of interest and a control antibody thereto) and after sufficient washing, flow cytometry is used for observation of the same.

Detected scattered light or fluorescence may be converted to electric signals and analysed by a computer. Results thereof allow lymphocytes, monocytes and granulocytes to be distinguish by determining the size of the cells from the intensity of FSC, and the intracellular structure from the intensity of SSC. Thereafter, the cell population of interest may be gated to study antigen expression patterns in the cells, if necessary.

Typical examples include separation of human peripheral monocytic leukocytes (mononuclear cell) and calculation of living cells, which may be conducted using flow cytometry with the following protocols:

Bleeding: A tourniquet is applied to a donor less than 5 cm from a venipuncture site, typically on an arm, and the veins are engorged for puncture. At this point, the donor is required to grip. The venipuncture sites are disinfected with ethanol, and bled. When 6 ml of blood is collected in a syringe the tourniquet is removed, the needle is removed quickly, and pressure is applied to the puncture sites with ethanol-soaked cotton. When bleeding has stopped to some extent, an adhesive bandage is applied to the puncture site. The needle is appropriately processed and the collected blood and heparin (or other anticoagulant) is mixed by several rounds of inversion of the syringe.

Blood dilution: Fifteen ml of blood obtained as mentioned above is transferred from the syringe to a 15-ml centrifuge tube. Six ml of PBBS (0.1% bovine serum albumin (BSA) containing phosphate buffered saline: composition: NaCl 7.20 g, KCl 0.320 g, Na₂HPO₄ 1.15 g, KH₂PO₄ 0.20 g, CaCl₂ 0.140 g, MgCl₂.6H₂O 0.20 g, MgSO₄.7H₂O 0.20 g, glucose 1.00 g, phenol Red 0.01 g. were added to make the total volume of 1 liter by adding an appropriate water), were added to double the volume of the blood (if the blood volume is less than 6 ml, then the PBBS to be added was increased to make the total volume to be 12 ml). Diluted blood was mixed by pipetting without bubbling.

Separation of mononuclear cells by specific gravity centrifugation method: Prepare two tubes of centrifugation tubes with lymphocyte separation solution (for example, Ficoll-Paque; 3 ml) in advance. Dilution blood prepared as described above was added to the lymphocyte separation solution by using a pasteur pipette, and was overlaid not to disturb the interphase between the separation solution and the diluted blood. The centrifugation tube with the separation solution with the diluted blood overlaid, was centrifuged at 1,500 rpm for 30 minutes.

Collection of mononuclear cells: Peripheral mononuclear cells including lymphocytes and monocytes, are observed between the plasma (yellow colored) and separation solution (transparent), as a white bandage-like layer. On the other hand, erythrocytes and granulocytes sedimented to the bottom of the centrifugation tube. White band-like mononuclear cell layer was recovered by using pasteur pipette, and was loaded in a 15 ml fresh centrifugation tube (for cell washing purpose). Mononuclear cell suspension recovered from the two centrifugation tubes are collected in a single washing centrifugation tube, and an appropriate volume of PBBS was added to the cell suspension to make 12 ml in total volume. After mixing by pipetting without bubbling, centrifugation was conducted for 10 minutes to pellet the cells. After centrifugation, supernatant was aspirated by an aspirator. Ten ml of PBBS was added to a cell pellet, cells were carefully suspended by Komagome pipette, and centrifuged for 1,500 rpm and 10 minutes, and the supernatant was aspirated to wash and remove the lymphocyte separation solution from the cells completely.

If necessary, live cell number is counted. The Supernatant was aspirated, leaving a cell pellet, and 3 ml PBS was added with a short pipette, and a pasteur pipette was used to resuspend the cells (agitate the mixture thoroughly so that no cell mass is found).

Fifty μl of the cell suspension in the centrifugation tube was removed with a micropipette equipped with a tip on the apex, this should be conducted quickly before the floating cells could sediment by gravity. The sample is transferred to an Eppendorf tube, and was carefully agitated by pipetting the cell suspension with 0.1% trypan blue solution (50 μl) prelocated in the tube.

This is, for example, placed on Burker-Turk blood counting frame with a coverslip, and micropipette is used to mix the trypan blue solution with the cell suspension, which is absorbed by capillary phenomenon. The volume of the suspension to be introduced between the counting frame and the coverslip, is about 7 μl, however, depending on the way the coverslip is equipped (the spatial relationship with the counting frame), the volume is appropriately adjusted. If the volume is appropriate, a tightly adhered interface between the counting frame and the coverslip will appear as a Newton's ring.

The Burker-Turk counting frame is mounted on a microscope, and the diaphragm of the microscope condenser is adjusted for better observation of graduation. Blood cell counting frames of this type possesses nine squares of 1 mm×1 mm. The centrally located square with tiny sections is used for counting erythrocytes. When leukocytes are counted, any larger square on the corner is used. It should be noted that the triple line sectioning the outer frame is the edge of the square of 1 mm×1 mm.

Cell number is determined by counting cells included in the 1 mm×1 mm square. Cells that are stained blue are dead cells, whereas those observed to be round and transparent are living cells. Both living and dead cells in the 1 mm×1 mm square are counted.

The frame is designed such that distance between the graduation face of the counting frame and the cover glass is 0.1 mm. Accordingly, the cell number counted herein is the number of cells suspended in liquid of a volume corresponding to a rectangular parallelepiped having a 1 mm×1 mm bottom face and a height of 0.1 mm.

If necessary, the concentration of the cell solution is adjusted. Cell density as calculated above is based to adjust the ultimate cell density to 1.0×106 cells/ml by adding PBS to the cell suspension. Cell suspension is stored in a refrigerator.

Human peripheral mononuclear cell suspension as prepared above (adjusted to 1×10⁶/ml), was used for the following analyses:

FITC labeled anti-CD antibody: for example FITC labeled anti-human CD4 antibody (adjusted to 10 μg/ml): 20 μl

PE labeled anti-CD antibody: for example PE labeled CD8 antibody (adjusted to 10 μg/ml): 20 μl

0.1% bovine serum albumin (BSA) containing phosphate buffered saline solution (PBS)

Micropipetter, tips for micropipetter, Eppendorf tubes, short pipettes, Pasteur pipette, Komagome type pipette, and the like.

Stream Current Type Aspirator

Microcentrifuge for Eppendorf tubes.

(Operation Procedures)

A) Two Eppendorf tubes are prepared and labeled as A and B on each cap. One ml of peripheral mononuclear cells prepared as described above are added to each tube (1×10⁶ cells per tube). This suspension is centrifuged in a microcentrifuge at 3,000 rpm for three minutes to pellet the cells, the supernatant is absorbed and removed.

B) Fifty μl of anti-CD4 antibody solution is added to tubes labeled A with a micropipetter equipped with a tip, and the same tip is used for pipetting without bubbling to resuspend the cells. Next, a fresh tip is placed on the micropipetter, and 50 μl of anti-human CD8 antibody solution, which is appropriate for the same tube, for example, is added thereto, and antibody solution and the cell suspension are vortexed and mixed in a similar manner. Thereafter, the tube is placed on ice to bind the antibody to the cell.

C) One hundred μl of anti-human CD19 antibody solution is added to tube labeled B, for example, by a micropipette, and the antibody solution and the cell suspension are vortexed and mixed in a similar manner, and the tube is placed on ice.

D) Thirty minutes after the tube has been placed on ice one ml of PBBS was added to each tube using a Komagome type pipette, and microcentrifuge was used to conduct centrifugation at 3,000 rpm for three minutes recover the cells.

E) One ml of PBS was added to each tube with cell pellet obtained after aspirating the supernatant therefrom, with a Komagome type pipette, taking care not to contact the cells with the tip. The cells is tube A were gently resuspended using a Pasteur pipette. Next, the cells in tube B were suspended with a fresh pipette. The tube was centrifuged in a microcentrifuge at 3,000 rpm for three minutes, and the supernatant removed.

F) Once the supernatant had been aspirated from each tube to leave a cell pellets, 0.5 ml of PBS was added with a Komagome-type pipette, taking care not to contact the tip with the cells. The tube was placed on ice.

G) The cell suspension was passed through a coarse filter to remove aggregated-cells and transferred to a tube for flow cytometry analysis, a cover was placed thereon. The tube with the cell suspension was again placed on ice.

H) A flow cytometer was used for analyzing 10,000 mononuclear cells, and data analysis software (for example, CellQuest(R)) was used for visualizing stained data. Tube A was double stained with FITC and PE, and thus analyzed by dot plotting, to investigate the ratio of CD4 positive cells and CD8 positive cells. Tube B is a single stained, and thus a histogram was used for analysis to investigate the cell surface CD19 positive cells.

(Diagnosis)

As used herein, the term “diagnosis” refers to determination of the status of a disease, disorder and condition of a subject by identifying a variety of parameters relating the disease, disorder and condition thereof. The method, device, apparatus, system and the like of the present invention are used to analyze the differentiation stages of a cell in the body, and such information is used to select a variety of parameters such as diseases, disorders and/or conditions in the subject, formulations or methods for treating or preventing the disease, disorder or condition, to be administered.

The diagnosis method of the present invention can make use of any material derived from the body in principle, and the present invention may be carried out by those other than healthcare professionals such as medical doctors and the like, and thus is industrially applicable.

As used herein the term “treatment” or “treat” refers to, when they are used for a disease or disorder, preventing deterioration of such a disease or disorder, preferably maintaining the status quo, and more preferably, relieving and most preferably extinction thereof.

As used herein, the term “subject” refers to an organism which is treated according to the present invention, also called a “patient”. A patient or a subject may preferably be a human.

As used herein, the term “disease” targeted by the present invention, refers to any disease relating to cell differentiation stage or in the treatment of which a cell differentiation stage is involved. Such diseases include, but are not limited to, anemia, cancer (such as, tumor, leukemia, lymphoma, and the like), immunological diseases and the like.

As used herein, the term “disorder” as targeted by the present invention, refers to any disorder relating to a cell differentiation stage.

Specific examples of such diseases, disorders or conditions include, but are not limited to, for example: circulatory system diseases (for example, anemia including aplastic anemia, in particular severe aplastic anemia, cancerous anemia and the like); cancer or tumor (for example, leukemia, multiple sclerosis and the like); immune system diseases (for example, T lymphocytes deficiency, leukemia and the like); respiratory system diseases (for example, lung cancer, bronchial cancer, and the like); digestive organ diseases (for example, primary liver cancer and the like); urinary organ system diseases (bladder cancer and the like); reproductive system diseases (male reproductive diseases such as prostate cancer, testicular cancer and the like, and female reproductive diseases such as uterine cancer and ovarian cancer, and the like) and the like.

As used herein, the term “cancer” refers to a condition in which there are malignant cells relating to an abnormality, cells proliferate more rapidly than normal cells, and are capable of infiltrating into peripheral tissues in a destructive manner, and/or capable of metastasizing, and/or a condition in which such malignant cells exist. In the present invention, cancer includes, but is not limited to, solid cancers and hematopoietic tumors.

As used herein the term “leukemia” or “hematopoietic tumor” are interchangeably used to refer to diseases associated with immature hematopoietic cells with tumoric proliferation. Depending on whether the proliferative leukocytes are myeloid or lymphatic, the leukemia is classified into myeloid leukemia and lymphotic leukemia.

As used herein the term “myelogenous leukemia” or “myeloid leukemia” are interchangeably used and clinically classified into acute and chronic. It is reportedly associated with tumoric proliferation and accumulation of immature leukemia, but also suppresses the proliferation and differentiation process of normal bone marrow cells. Chronic myeloid leukemia is believed to be derived from tumorigenesis of myeloid stem cells, and the tumor cells have pluripotency. A number of tumor cells with a variety of differentiation stages are observed.

As used herein, the term “chronic myeloid leukemia” is believed to be derived from tumorigenesis of myeloid stem cells. A number of tumor cells with a variety of differentiation stages are observed. Thus, determination of a variety of differentiation stages is necessary for tailor-made therapy.

As used herein the term “acute myeloid leukemia” consists of tumorigenic proliferation, and as specific forms, there is acute pre-myeloid leukemia, acute monocytic leukemia and the like. It attacks the bone marrow, liver, spleen and the like as in the chronic myeloid leukemia, and generally, prognosis failure is observed. Acute myeloid leukemia may reportedly be familiar, and is known to be associated with a variety of chromosome abnormalities. Accordingly, determination of a variety of differentiation stages is necessary for tailor-made therapy. In particular, in most cases, amongst three lineages erythrocytes, leukocytes and platelets, one to three lineages will be reportedly reduced. Therefore, the patients will be susceptible to anemia or infectious diseases, and hemorrhagic. Therefore, therapies according to the lineage are important.

As used herein the term “lymphocytic leukemia” are commonly observed in a juvenile, and most of them are acute lymphocytic leukemia. Tumor cells are classified into those exhibiting properties of B lymphocytes and T lymphocytes, and those having no such properties. Adult T cell leukemia, which are often observed in Kyushu area in Japan or Caribbean Coast countries, is chronic lymphatic leukemia which is often seen in adults around 50 years old. HTLV-I appears to infect to pateitn in the same manner with respect to CD4 lymphocytes. The major route of infection route is feto-maternal infection, and at present in Japan, there are about one million carriers thereof, and it is believed that one in about 100 has adult T lymphocytic leukemia. Therapies therefor include administration of metabolism antagonists, alkylating agents, a portion of antibiotics, plant alkaloids, aderenocortical hormones, and the like.

Classification of hematopoietic tumors, particularly leukemia, has been performed by morphological or cytochemical classifications by French-American-British (FAB) classification. However, these days, detailed classification has been increasingly conducted by means of characteristic chromosomal abnormalities that reflect the disease state or etiology of leukemia (WHO classification). However, leukemia consists of an extensive variety of types of diseases, and it is difficult to properly understand the property of leukemia cells by these methods of classification. Accordingly, surface antigens, immunological gene analysis, search for specific gene variation and the like are important not only for properties of leukemia cells but also important during consideration of therapeutic strategy. Therefore, use of the present invention to analyze the same and obtain the results thereby is extremely important. Study of surface antigen expression patterns of a tumor cell using flow cytometry is now an essential check for chronicity of cell, cell origin, diagnosis, therapy, prognosis prediction and the like. Therefore, it is necessary to fully understand the advantages and disadvantages of the check.

Such advantages include not only shorter time necessary for multiple cells, but also enablement of the simultaneous analysis of a plurality of antigens expressed on a single cell by multiple staining. In order to solve this limitation, CD45 blast gating method has been introduced (see, T. Miyazaki et al., Gating method using CD 45 antibody in an acute leukemia surface marker analysis by flow cytometry method, Rinsho Ketsueki (Clinical Blood) 37: 214-220, 1996). CD45 antigen is a common leukemia antigen, and thus is expressed in all lineages of leukocytes. Differences in expression amount are recognized, depending on a variety of each lineage. Further, the expression level thereof is poor in leukemia in immature differentiation/mature stages, and increases with the advance of differentiation/maturation.

Expression amount of CD45 antigen on such cellular surfaces is used to classify cells in bone marrow blood to determine the lineage thereof and differentiation/maturation stages, allowing analysis of the expression pattern of cellular surface antigens in detail. However, tumor cells with little proliferation have a high level of CD45 expression, and thus it should be noted that activated T lymphocytes appear during virus infection, or CD45 weak positive expression normal cells in bone marrow during the recovery after chemotherapy. In acute myeloid leukemia (AML), there is correlation between data representation by CD45 blast gating method and FAB classification. On the other hand, it has been reported that, in acute lymphoblastic leukemia (ALL), not only has no definite pattern in the expression intensity of CD45 antigen be observed, but also cases CD45 negative have been observed.

Search for leukemia cells can be conducted after leukocyte separation by hemolysis of bone marrow liquid or peripheral blood, or after separation of monocytes using gravity centrifugation, by means of flow cytometry or surface antigen expression patterns. For diagnosis, monoclonal antibodies against CD antigens relating to the lineage or stage of cells are used. In particular, during analysis of leukemia cells using FCM, it is important which CD antigens are used in which combination. It is important to produce an analysis panel (combination of antibodies) after consideration of the characteristics of leukocyte cells.

Typical antigens for stem cells include CD34 and CD117, antigens for myeloid lineage include CD33, CD13, CD15, CD16, CD11b, antigens for monocytic lineage include CD14, antigen for B lymphocytic lineage include CDI9, CD10, CD20, CD21 and CD22, antigens for T lymphocytic lineage includes CD7, CD2, CD3, CD4, CD8, CD1a, and antigens for Natural Killer cell lineage include CD56, which are used for determining the lineage of leukemia cells in an antigen expression manner. In addition, other antigens identified by the present invention may be used.

Further, expression of cytoplasmic myeloperoxidase (MPO), CD3, CD22 and CD79, clonal reconstitution of immunoglobulin genes and T lymphocyte receptor genes, and the results of chromosomal analysis and the like are combined to allow more appropriate diagnosis. In the cases where AML and ALL are analyzed, production of panels in view of the clarification of lineages of leukemia cells, allows the classification of disease types in detail.

There are some cases where leukemia cells express surface antigens across two or more lineages, which is called lineage infidelity or lineage promiscuity. Production of an analysis panel by using CD antigens specific to individual leukemia cells, allows the detection of minimal residual disease (MRD).

Aberrant antigen expression leukemia, which is a representative leukemia thereof, is briefly described below for in terms of the cellular and clinical properties thereof.

CD7+acute myeloid leukemia (AML): CD7 is a pan-T lymphocyte antigen, and the expression thereof is found in T lymphocytic leukemia and also expressed in a portion of AML. Accordingly, if both MPO and intracytoplasmic CD3 are negative, it is necessary to monitor both AML (M0) and T-ALL, and it should be noted. CD7 expression is found in about 40% of AML, and the expression thereof is reportedly as possibly being a bad prognostic factor. In another study, there is no prognostic difference. Probably, CD7+AML is a variety of leukemia collection and thus the expression thereof appears at high frequency not only in de novo AML but also in AML transited from MDS. Therefore, it is inferred that these MDS/AML are intractable, and thus affects the pathology of CD7+AML. Further, in examples where chronic myeloid leukemia is converted to acute myeloid leukemia, there has been reportedly found CD34 and CD7 expression. This appears to mean that when cells of a stem cell type convert into tumor, then CD7 will appear.

CD19⁺AML: CD19 is a pan-B lymphocyte antigen, and is also sometimes expressed in AML as in CD7. Unlike CD7+AML which is a collection of a variety of leukemia cells, most of CD19+AML have chromosomal abnormality t(8;21)(q22;q22), and most of them are classified as FAB classification M2. CD19 AML is often found in adults, leukemia cells have a tendency for differentiation, and characterized by eosinophil proliferation and good prognosis. Further, according to WHO classification, it is classified as a single disease with good prognosis. However, there is a bad prognosis sub-group, and is suggested that CD56 expression, existence of addition chromosomal abnormalities and extramedullary tumor growth, and the like, are bad prognostic factors.

CD56⁺AML: CD56 is an antigen that appears on natural killer cells, and is sometimes expressed in AML cells. As described above, CD56 expression in t(8;21)AML is potentially suggested to be a bad prognostic factor, and appears to be a bad prognostic factor for therapy using all-trans retinoic acid against AMLM3.

CD15⁺CD117⁺AML: CD15 is expressed in granulocytes and monocytes, whereas CD117 is a stem cell antigen, and is expressed in undifferentiated cells. Expression of both is not observed in any single identical normal cell, however, a portion of AML-cells express both antigens, and leukemia with the above expression has a good prognosis.

CD33/CD13⁺acute lymphoblastic leukemia (ALL): CD33 and CD13 are both myeloid antigens, and are also expressed in ALL, which is called myeloid antigen+ALL. The expression machinery is unknown, but there is reportedly no difference in clinical pathology. Further, CD2, a T lymphocyte antigen, is sometimes observed in B-ALL, and the B-ALL reportedly has a good prognosis.

CD4⁺leukemia: CD4 is one of T lymphocyte antigens, and often found in T lymphocytic tumors of leukemia cells. However, it is also sometimes observed in MPO positive leukemia (CD4⁺AML). Most these leukemias are M0, M1, M4 and M5 (FAB classification), and it is possible that it is a type of tumorigenesis of undifferentiated monocytic cells. However, it is not clear whether or not CD4⁺AML exhibits pathology specific to the disease, and further analysis is necessary. On the other hand, there a CD4⁺CD56⁺Leukemia has been reported, which is MPO negative, and negative to CD3, CD117, CD33 and CD13 antigens. This implies leukemiation of dendritic cells (melanocyte). This type of leukemia is susceptible to extramedullary (lymph node and the skin) recurrence.

CD56⁺CD33⁺CD7⁺leukemia: This leukemia is MPO negative, and is reported to be one of a group of leukemias having CD56+CD33+CD7+CD-phenotype (myeloid/NK cell acute leukemia). These leukemias are inferred to potentially be a bad prognostic disease group, having a high frequency of extramedullary tumor growth, such as in the lymph nodes.

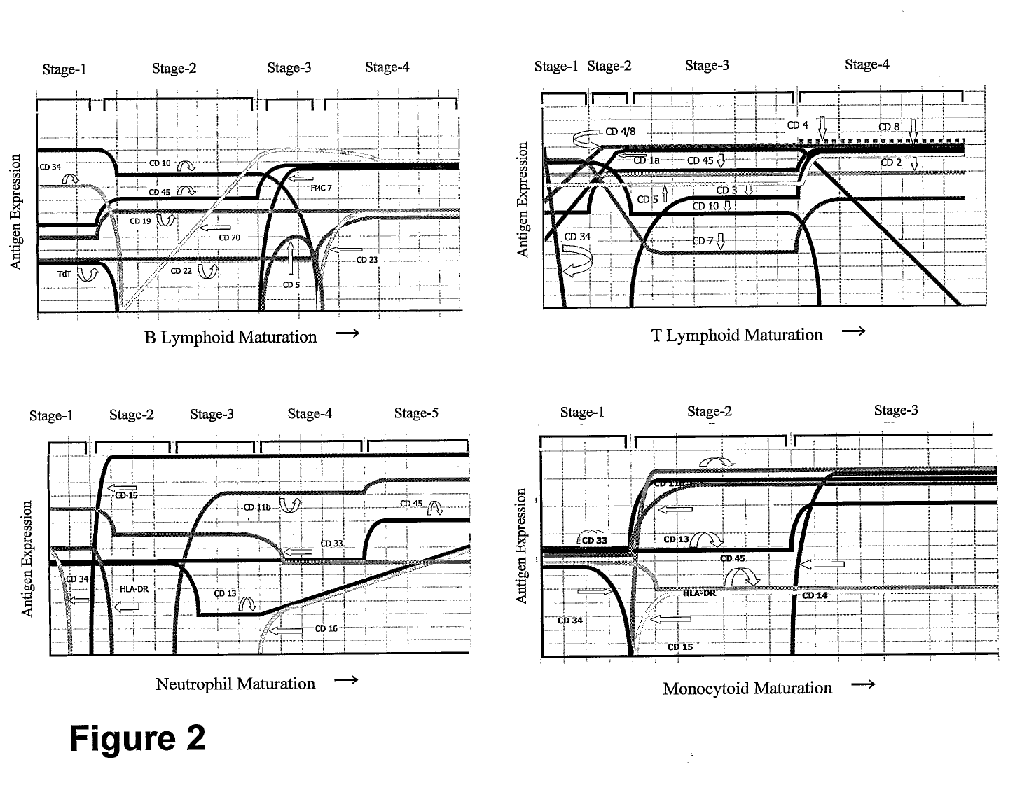

AML and ALL cells based on antigen expression lineage failure may be analyzed using flow cytometry for MRD analysis. Such an analysis clarifies aberrant antigen expression present in leukemia cells but not present in normal bone marrow blood or normal peripheral blood cells, which is indicative of antigen expression irregularity, antigen depletion, antigen expression volume abnormality, and antigen expression lineage infidelity. Cases in which antigen expression lineage infidelity is recognized, allow detection of MRD using antigen expression lineage infidelity as a marker. On the other hand, 85% of cases of AML express myeloid antigens (CD13, CD33, CD34 and HLA-DR) and lymphoid antigens or NK cell antigens (CD2, CD5, CD7, CD10, CD19, CD25 and CD56) simultaneously. In ALL, in 113 cases, myeloid antigens are expressed. Specific markers include Philadelphia (Ph1) chromosome positive, and in ALL, there are CD19⁺/KOR⁻SA3544⁺cells found, which are not present in normal bone marrow blood, in almost all the cases. This antigen expression lineage infidelity is considered to be a specific marker for leukemia cells, and thus MRD detection with high accuracy and high sensitivity can readily be attained by the above as markers (see FIG. 2).

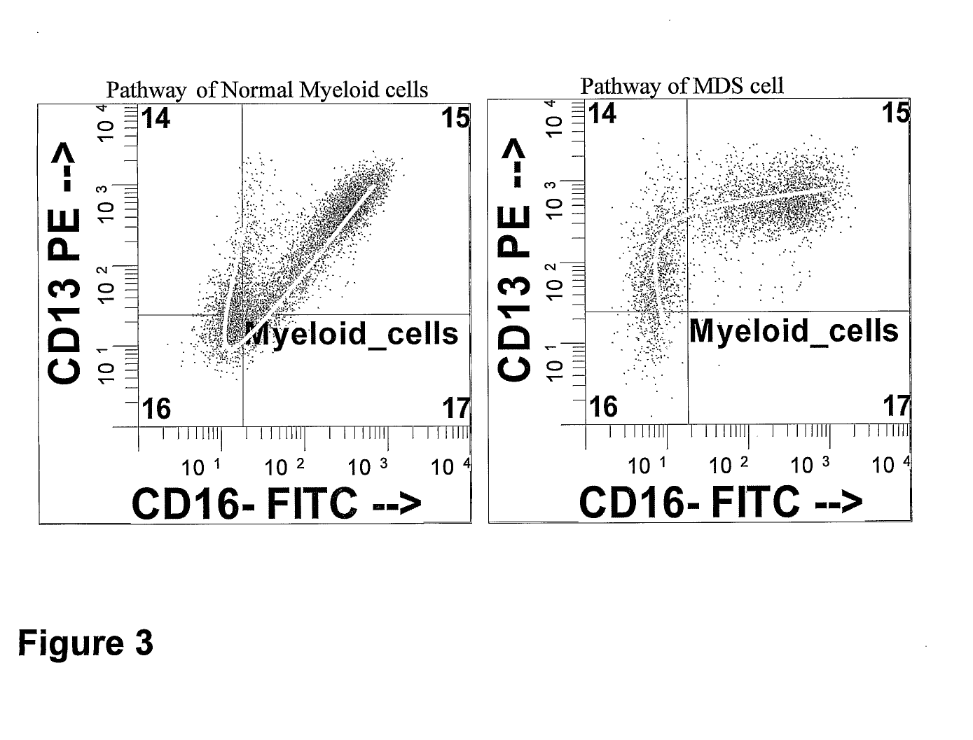

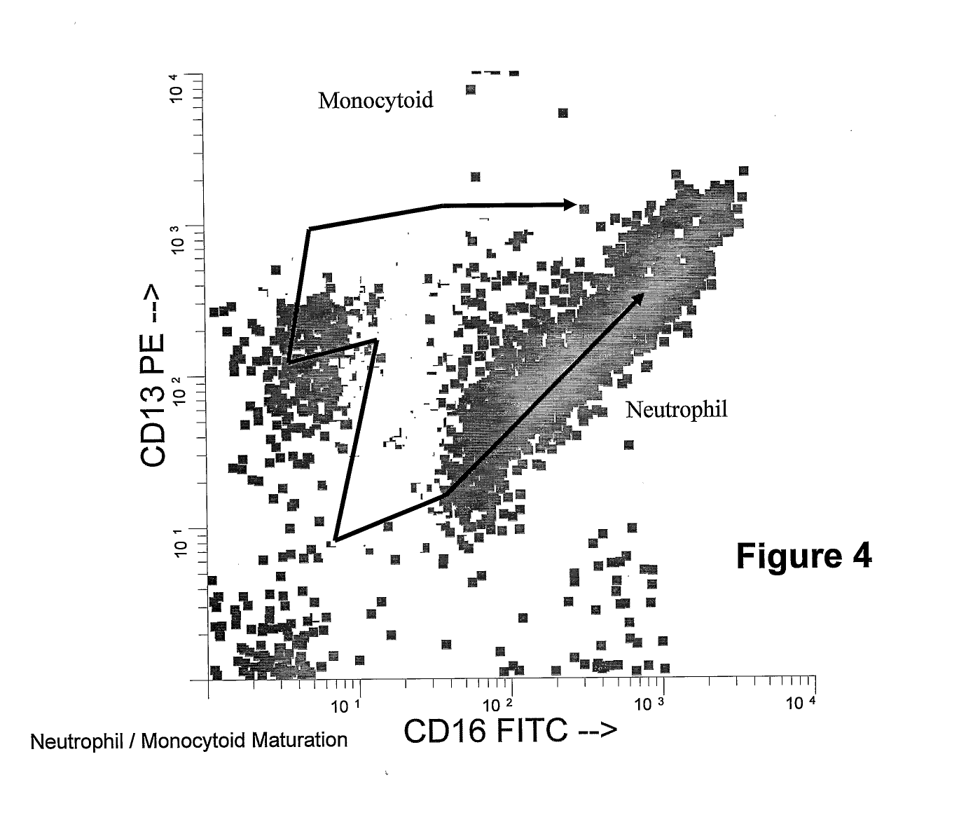

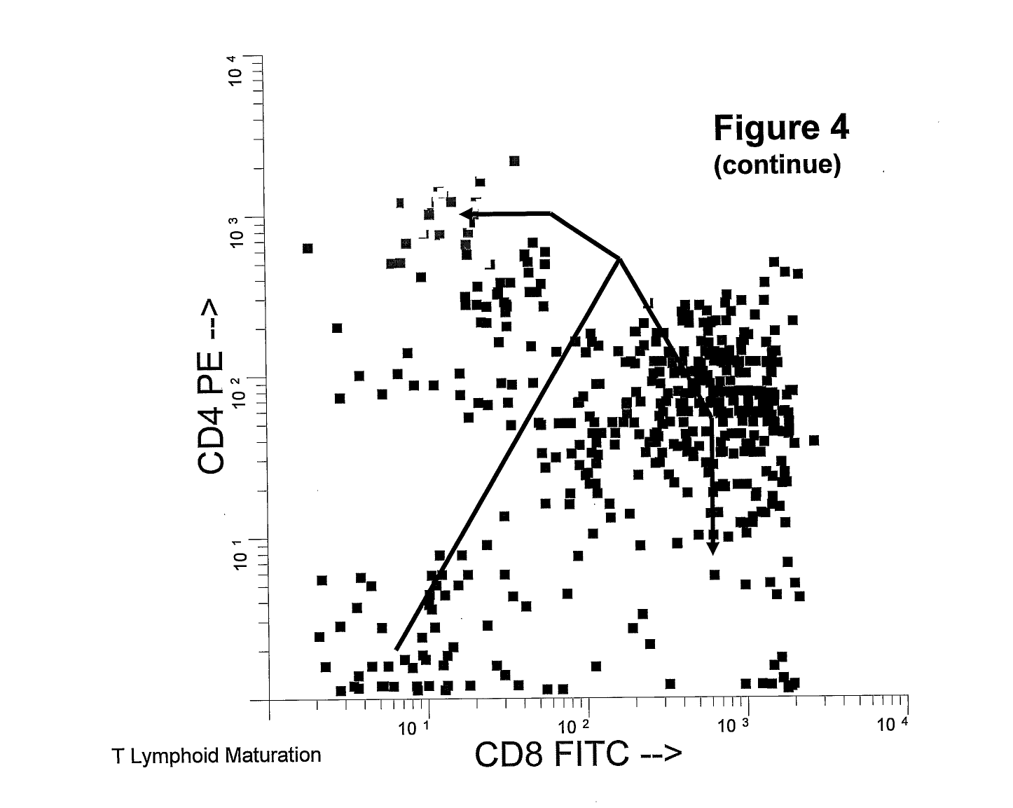

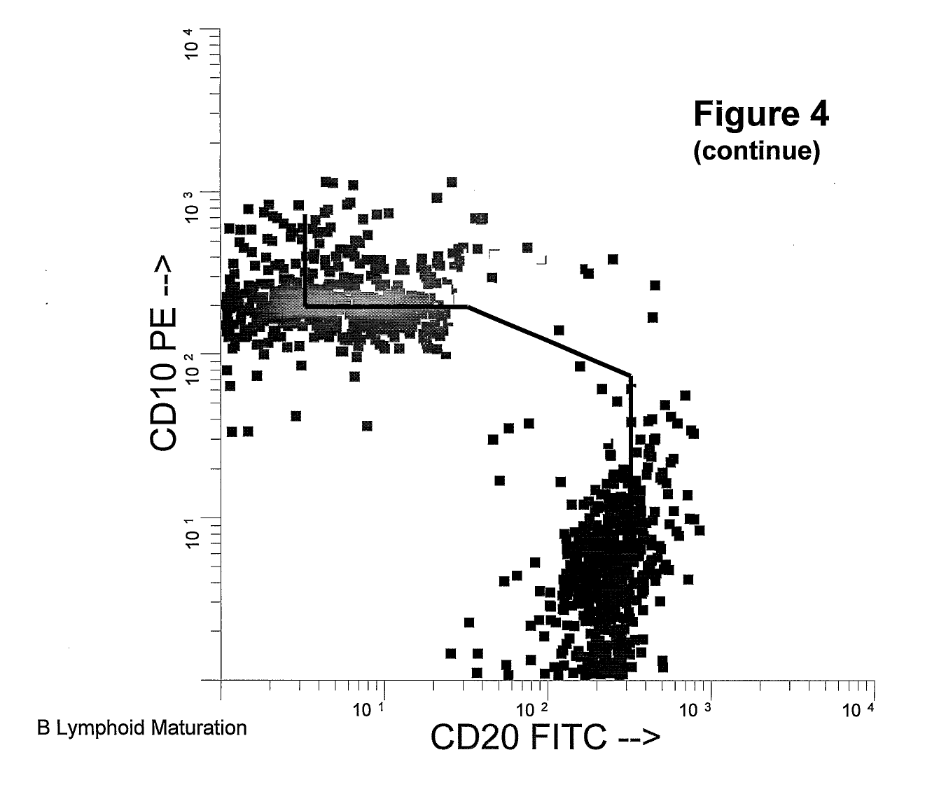

Myelodysplastic syndromes (MDS) cell analyses using FCM based on antigen expression infidelity, antigen deletion, and antigen expression abnormality may be typically conducted as follows: in the case of MDS as a target, clarification of maturation asynchrony of leukemia cells, antigenic absence, and quantitation abnormalities are considered for preparing a panel for analysis. Blood cells in normal bone marrow blood are matured through a differentiation/maturation process in an accurately regulated manner (see FIG. 3). Specific CD antigens are combined to quantitate and analyze distribution of a variety of antigens of normal cells present in bone marrow blood, peripheral blood and lymph node to result in a single lineage or normal pathway with expression and extinction of cellular surface antigens. This normal pathway is recognized in all blood cells, and his not influenced by age or therapy, and thus shows the same pattern in all individuals if they have normal cells. Such data provides the basis for analysis of a variety of leukemia cells, and it is understood that leukemia cells never exhibit the same antigen distribution as normal cells. That is, when the expression level of each antigen is measured, and plotted on multi-dimensional space, leukemia cells are always located in a different space to normal cells. This is because leukemia cells have abnormal antigen expression, as described above. MDS is difficult to analyze by studying whether the CD antigens are positive or negative in order to distinguish from normal cells. However, if the normal pathway is used as in the present invention for analysis, then the detection thereof is achieved (see FIG. 4). The following table shows exemplary panels for capturing these.

TABLE 2

*B lymphocyte maturation
CD20 × CD10 × CD34 × CD45
CD5 × CD19 × CD34 × CD45
CD10 × CD19 × CD34 × CD45
CD10 × CD20 × CD5 × CD45
CD19 × CD23 × CD5 × CD45
*T lymphocyte maturation
CD1a × CD3 × CD10 × CD45
CD1a × CD3 × CD34 × CD45
CD4 × CD8 × CD10 × CD45
CD4 × CD8 × CD7 × CD45
*bone marrow cell/monocyte maturation
HLA-DR × CD11b × CD10 × CD45
CD16 × CD13 × CD33 × CD45
CD16 × CD13 × CD11b × CD45
CD16 × CD14 × CD10 × CD45
CD14 × CD33 × CD11b × CD45
CD4 × CD10 × CD34 × CD45

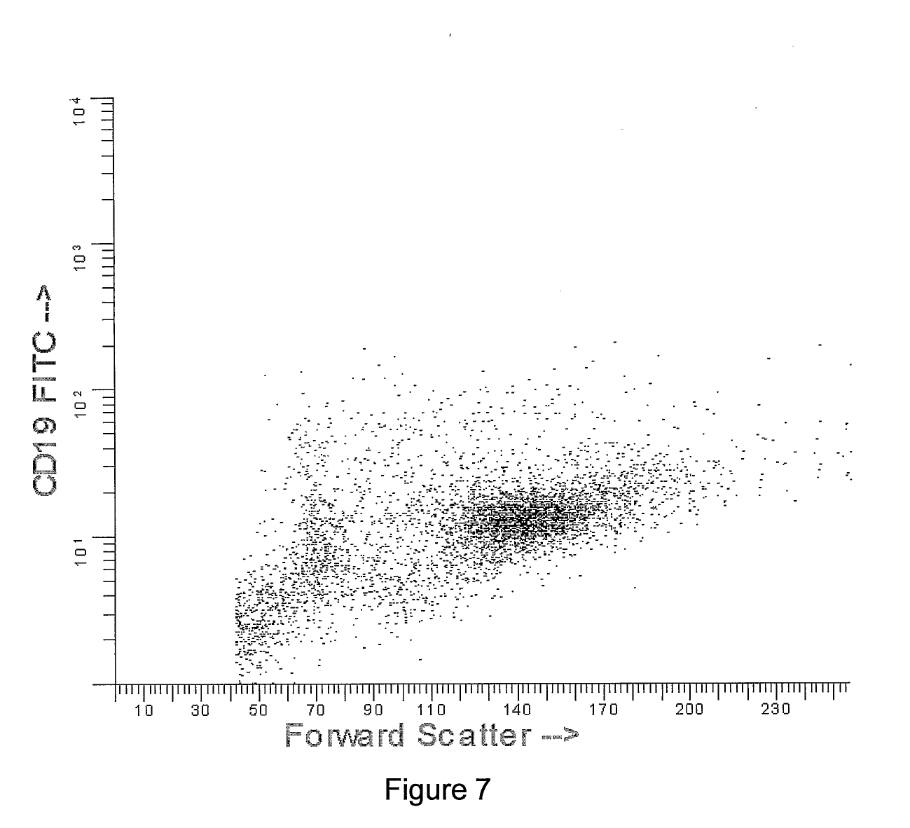

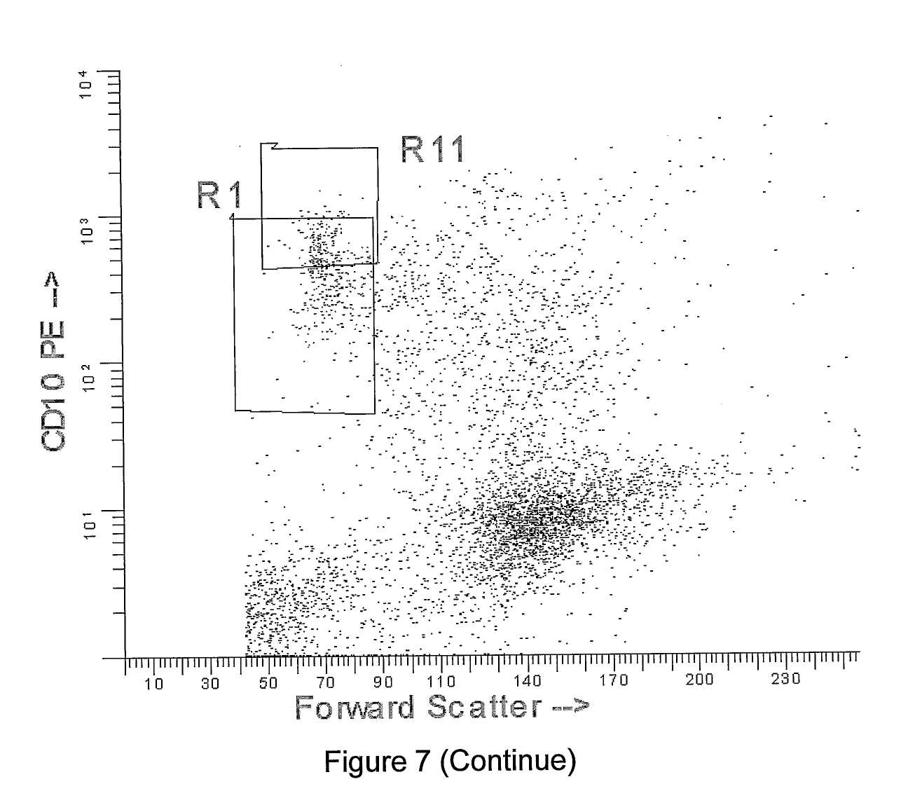

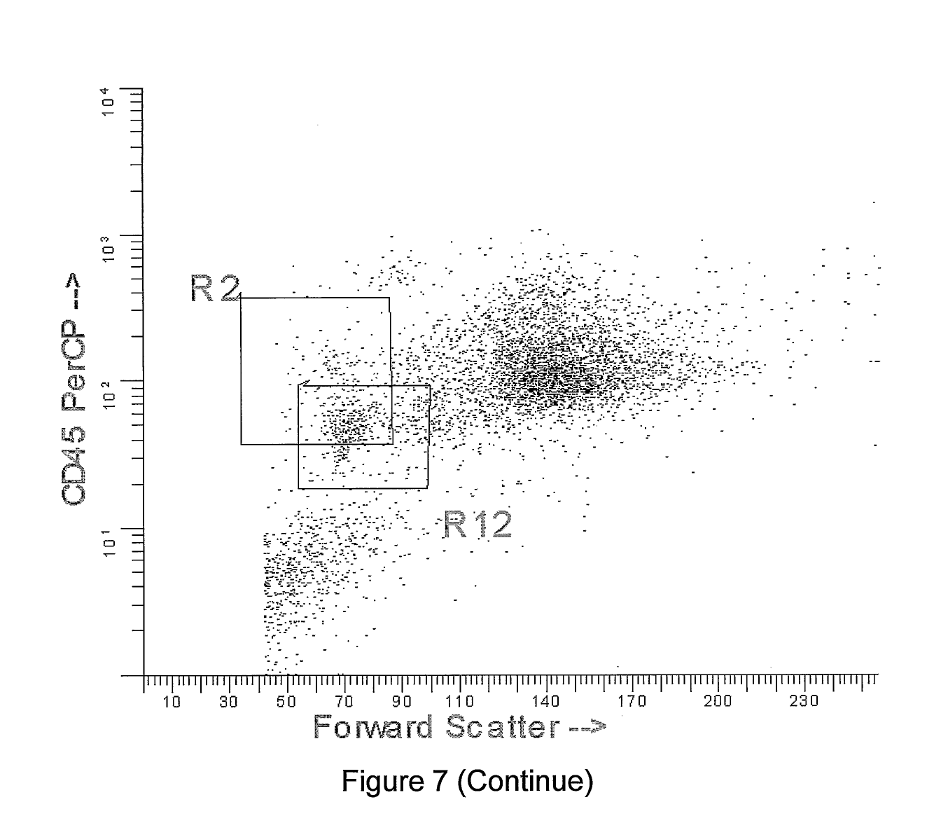

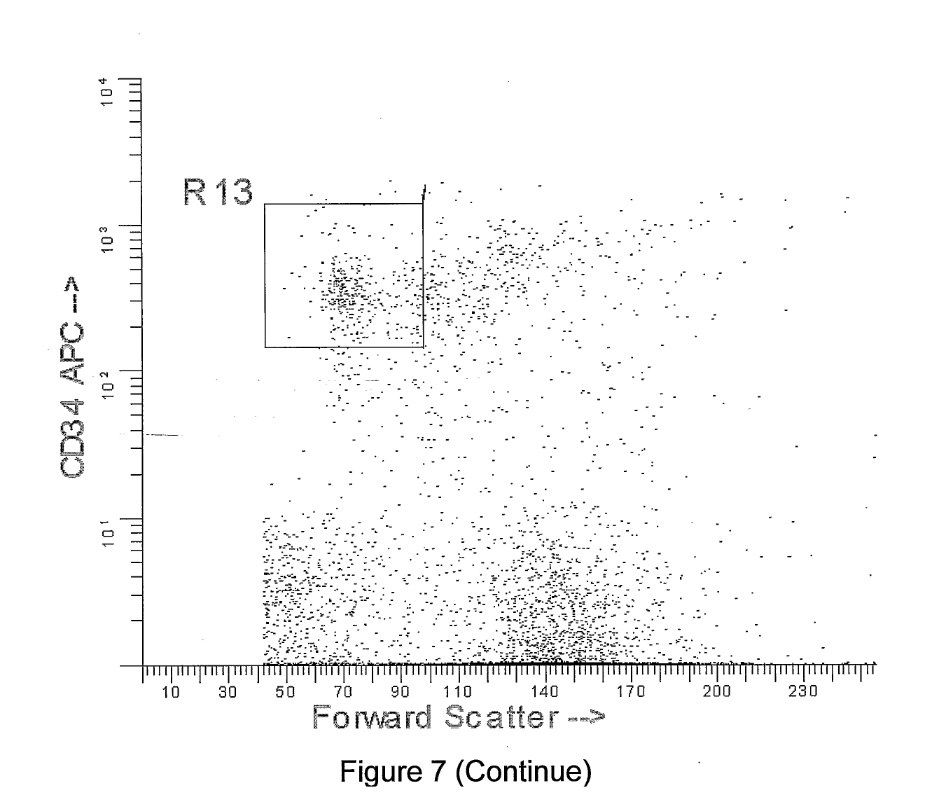

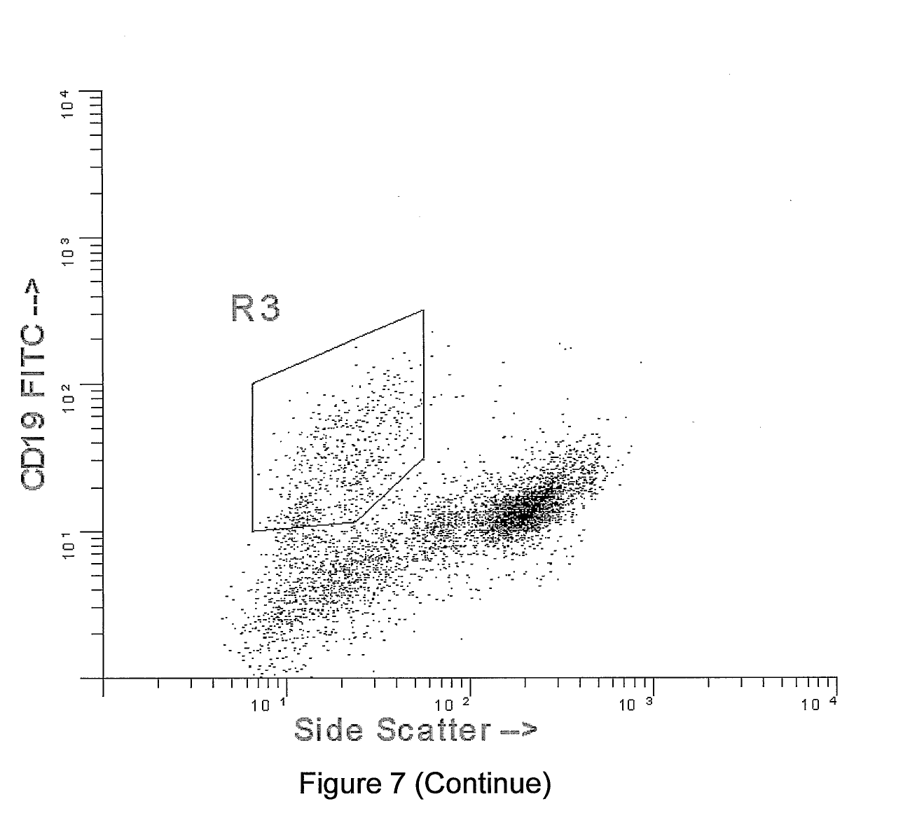

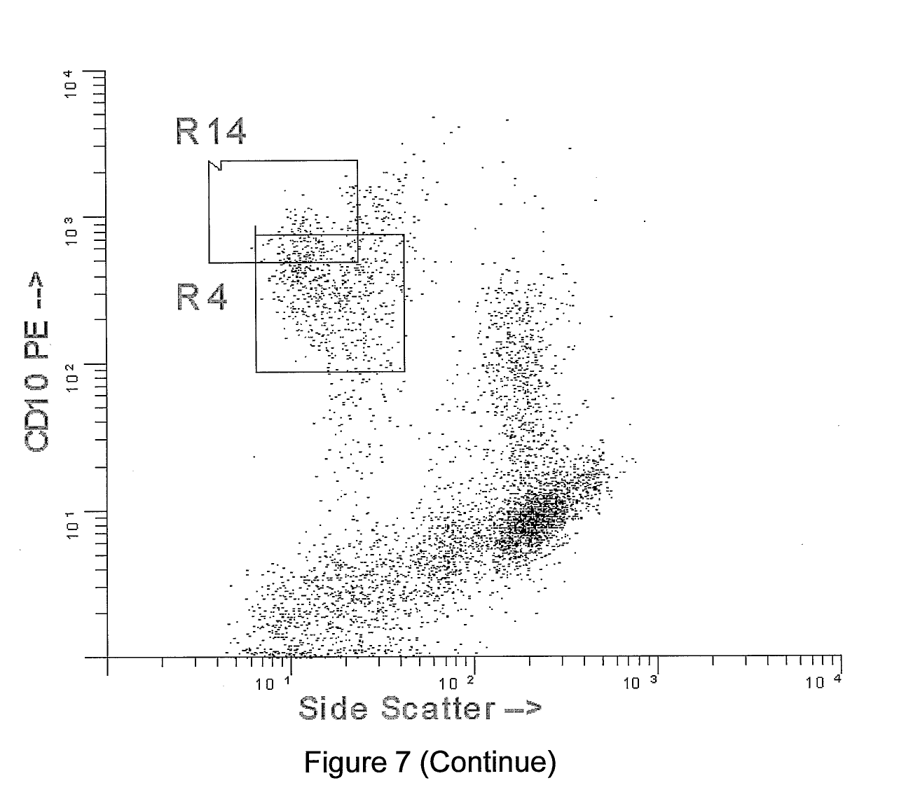

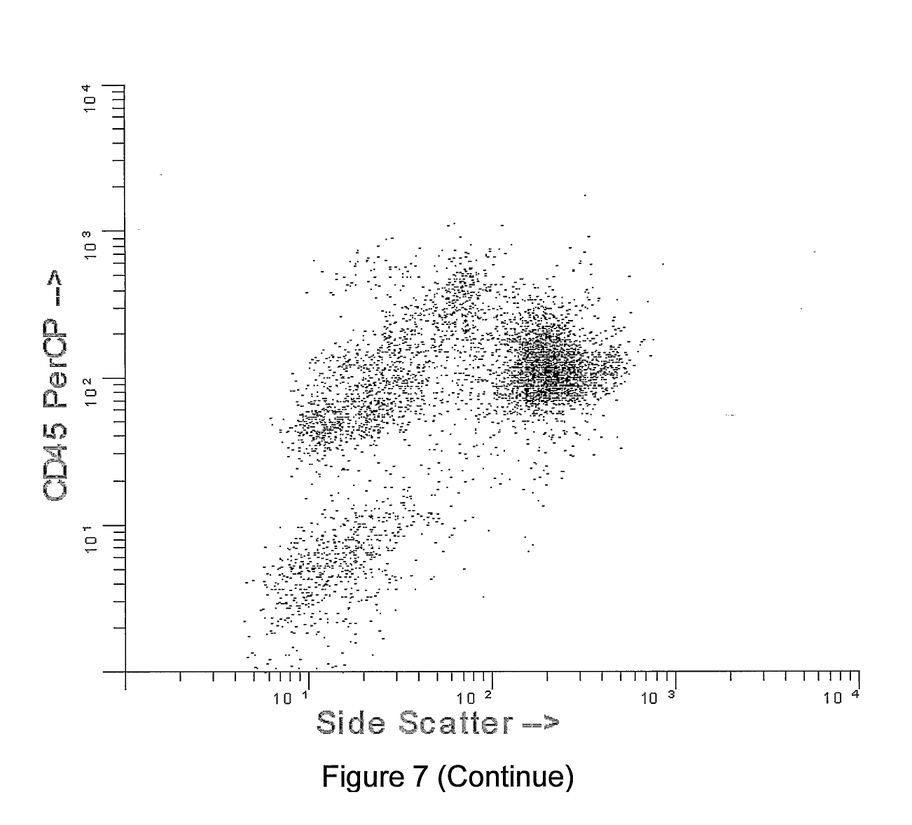

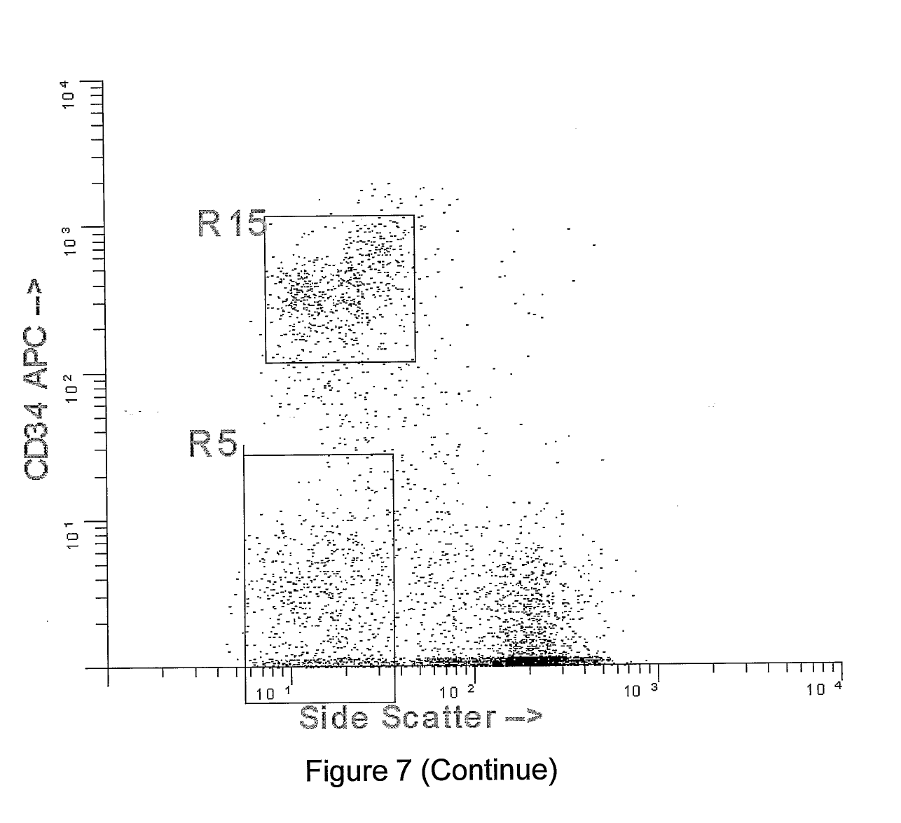

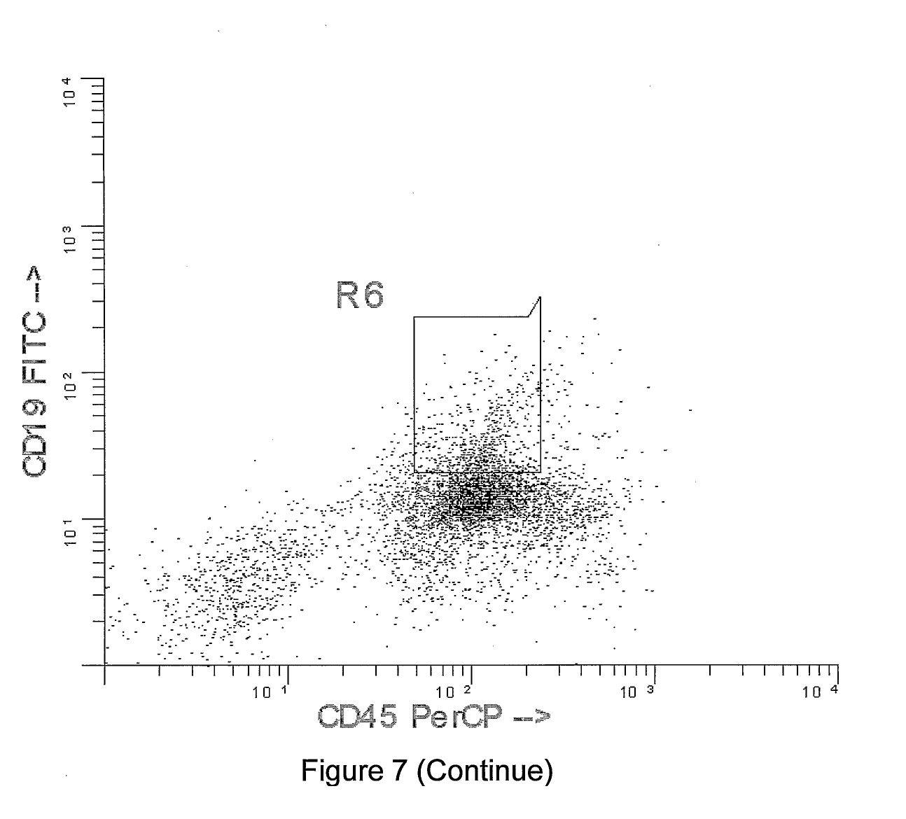

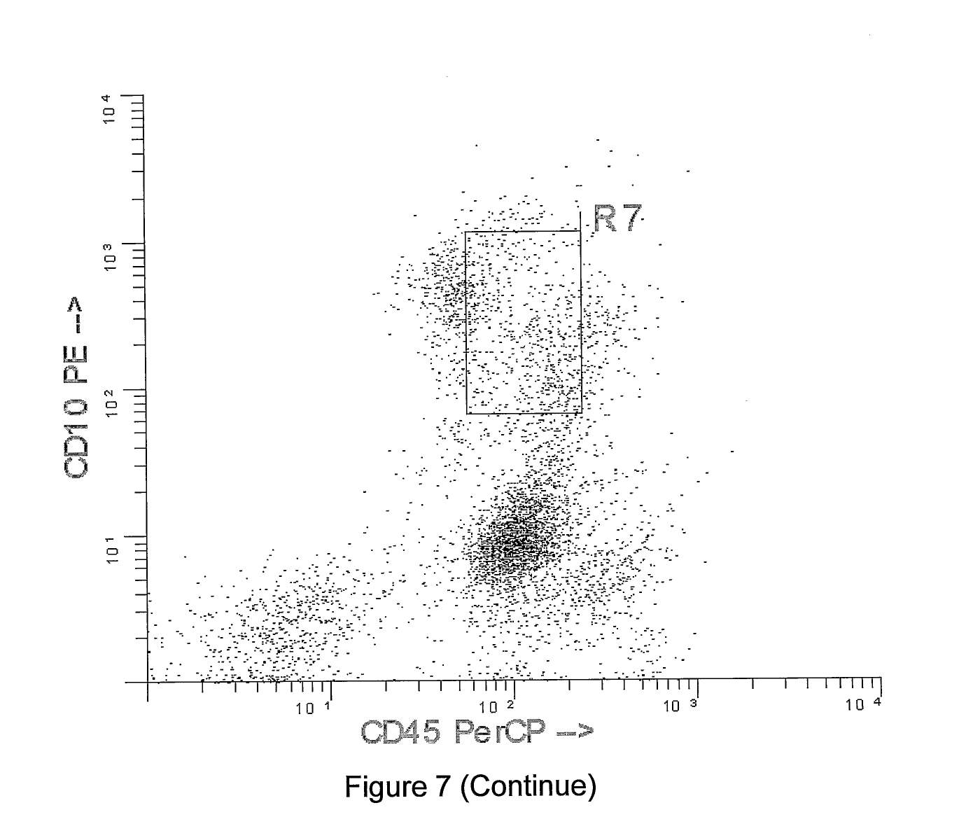

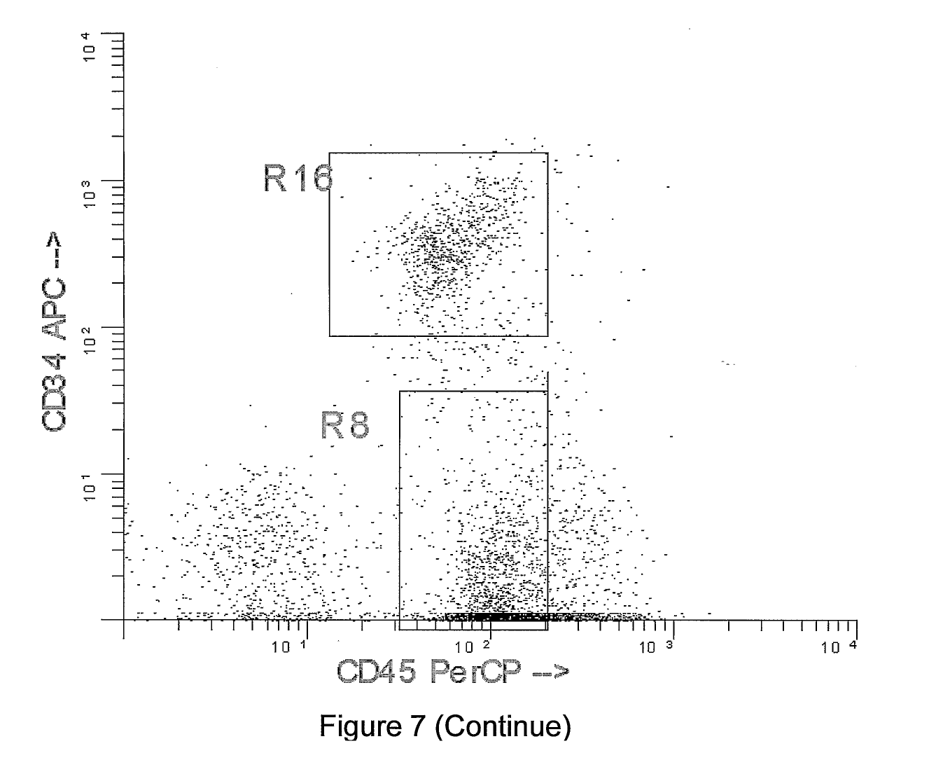

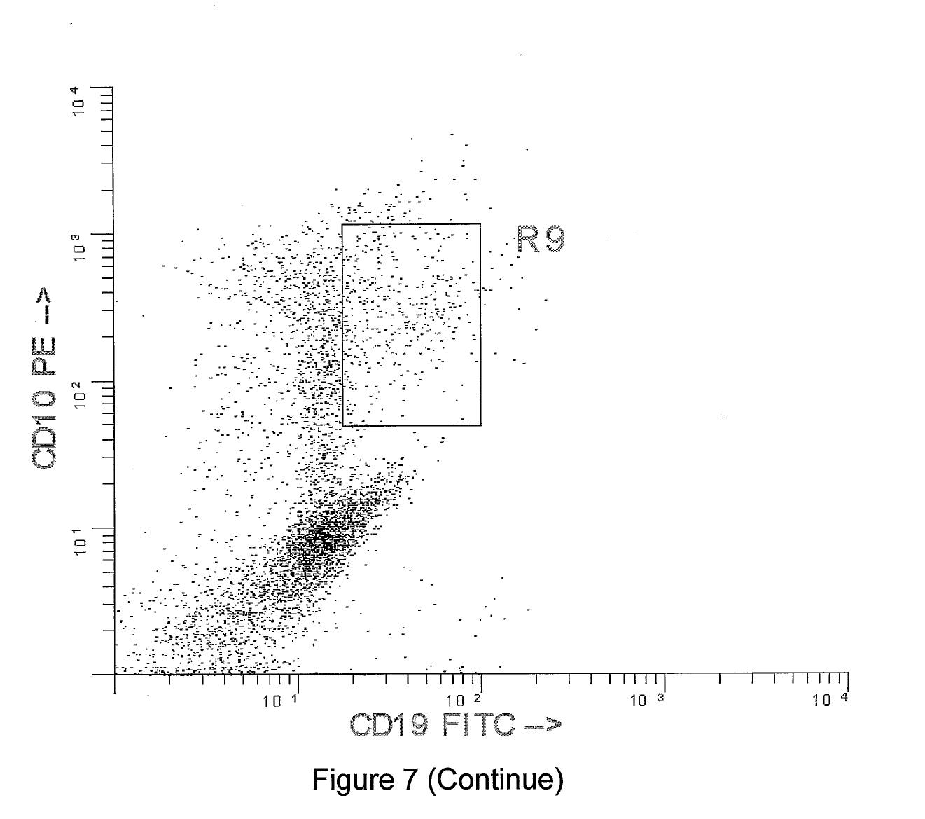

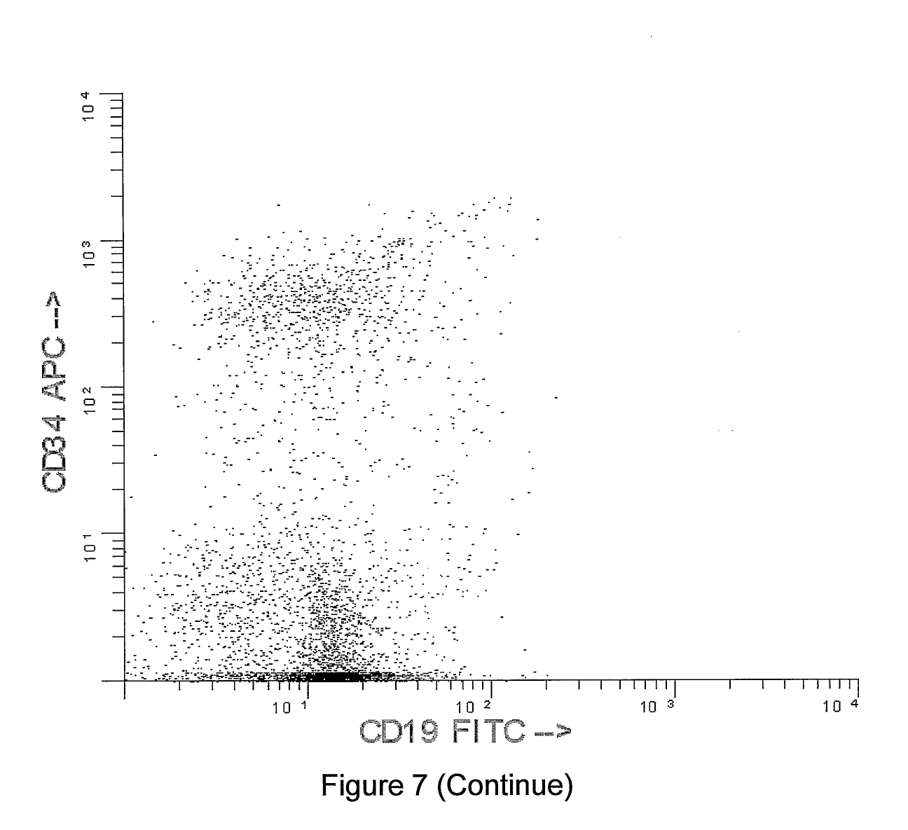

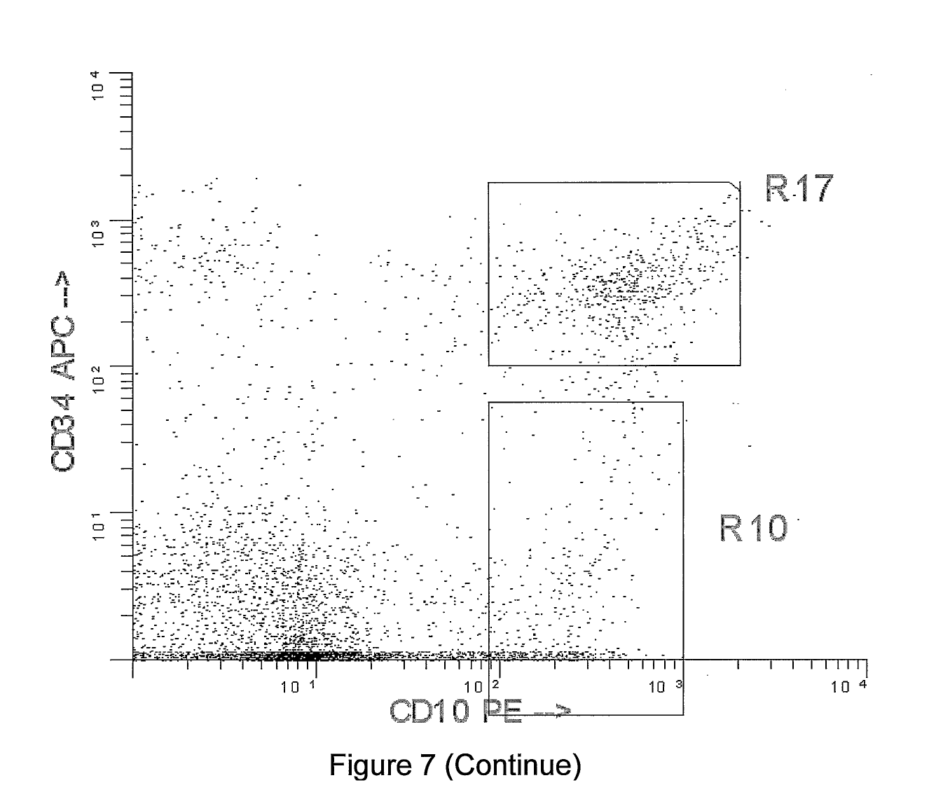

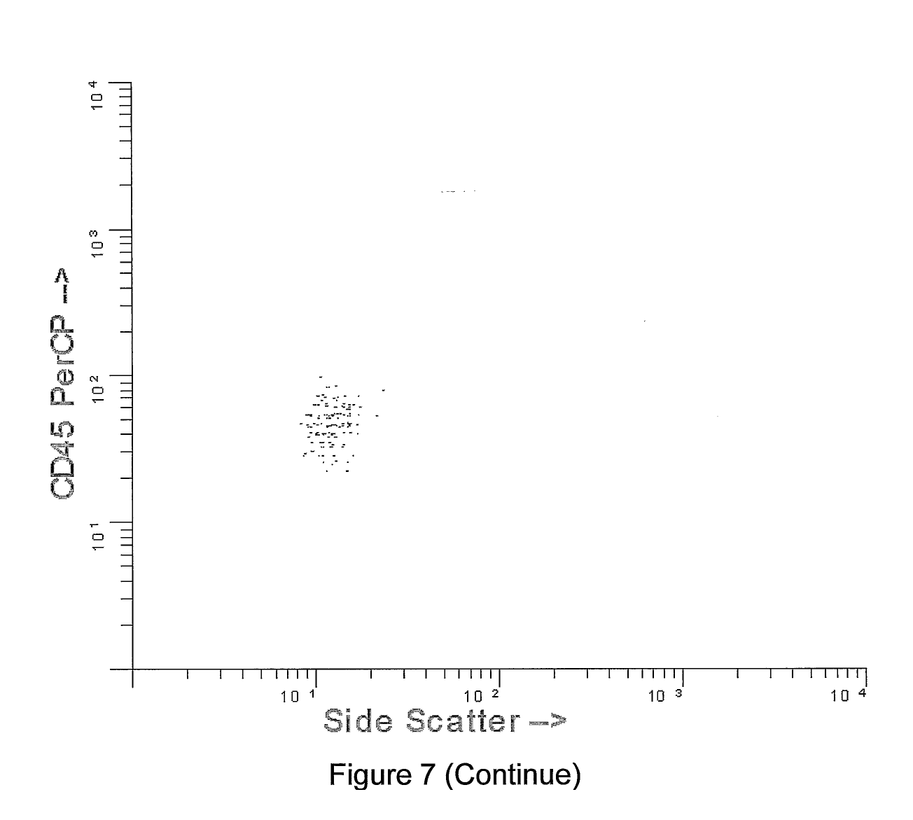

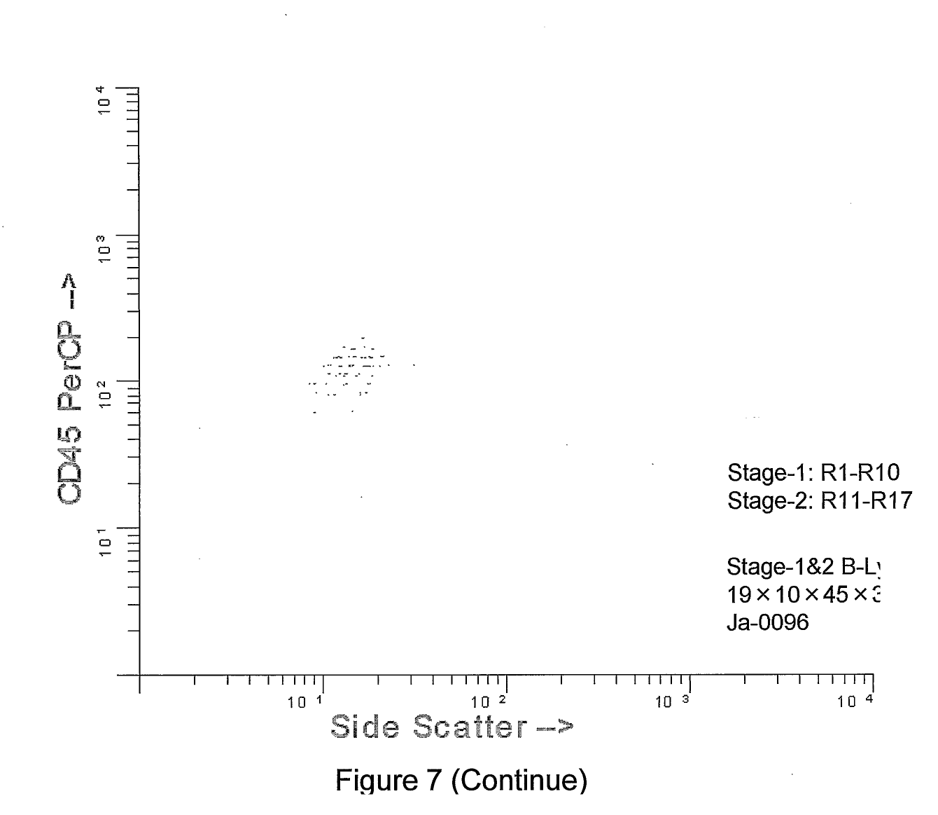

Each combination of the CD antigens allows detection of pathways reflecting the differentiation/maturation stages of each blood cell (FIG. 7). This panel is useful for the cases where AML and ALL are analyzed. Searching for surface antigen expression using the flow cytometry of the present invention is significantly useful in understanding the properties of leukemia cells.

As used herein, the term “kit” refers to a unit typically comprising two or more sections which provide portions (e.g., a reagent, an enzyme, a template nucleic acid, a standard, etc.). When components are not provided as a mixture and are preferably mixed immediately before use, this form of the kit is preferable. Such a kit advantageously comprises instructions which state how to treat the provided portions (e.g., a reagent, an enzyme, a nucleotide, a labeled nucleotide, a nucleotide (and its triphosphoric acid) arresting an extension reaction, a template nucleic acid, a standard, etc.). As used herein, when a kit is used as a reagent kit, the kit typically comprises reagent ingredients, a buffer solution, a salt condensate, an auxiliary means for use, instructions stating the usage, and the like.

As used herein, the term “instructions” refers to a description of a method for use or a reaction method for a reagent of the present invention, for the user. Instructions state a procedure for an enzyme reaction of the present invention. The instructions are prepared in accordance with a format defined by an authority of a country in which the present invention is practiced (e.g., Health, Labor and Welfare Ministry in Japan, Food and Drug Administration (FDA) in the U.S., and the like), explicitly describing that the instructions are approved by the authority. The instructions are a so-called package insert and are typically provided in paper media. The instructions are not so limited and may be provided in the form of a film attached to a bottle, and electronic media (e.g., web sites, electronic mails, and the like provided on the internet).

Molecular biological techniques, biochemical techniques, and microorganism techniques as used herein are well known in the art and commonly used, and are described in, for example, Sambrook J. et al. (1989), Molecular Cloning: A Laboratory Manual, Cold Spring Harbor and its 3rd Ed. (2001); Ausubel, F. M. (1987), Current Protocols in Molecular Biology, Greene Pub. Associates and Wiley-Interscience; Ausubel, F. M. (1989), Short Protocols in Molecular Biology: A Compendium of Methods from Current Protocols in Molecular Biology, Greene Pub. Associates and Wiley-Interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates; Sninsky, J. J. et al. (1999), PCR Applications: Protocols for Functional Genomics, Academic Press; Special issue, Jikken Igaku [Experimental Medicine] “Idenshi Donyu & Hatsugenkaiseki Jikkenho [Experimental Method for Gene Introduction & Expression Analysis]”, Yodo-sha, 1997; and the like. Relevant portions (or possibly the entirety) of each of these publications are herein incorporated by reference.

DNA synthesis techniques and nucleic acid chemistry for preparing artificially synthesized genes are described in, for example, Gait, M. J. (1985), Oligonucleotide Synthesis: A Practical Approach, IRL Press; Gait, M. J. (1990), Oligonucleotide Synthesis: A Practical Approach, IRL Press; Eckstein, F. (1991), Oligonucleotides and Analogues: A Practical Approach, IRL Press; Adams, R. L. et al. (1992), The Biochemistry of the Nucleic Acids, Chapman & Hall; Shabarova, Z. et al. (1994), Advanced Organic Chemistry of Nucleic Acids, Weinheim; Blackburn, G. M. et al. (1996), Nucleic Acids in Chemistry and Biology, Oxford University Press; Hermanson, G. T. (1996), Bioconjugate Techniques, Academic Press; and the like, related portions of which are herein incorporated by reference.

### DESCRIPTION OF THE BEST MODE FOR CARRYING OUT THE INVENTION

Hereinafter, the preferable embodiments of the present invention are described. The following embodiments are only provided for better understanding of the present invention, and it should be understood that the scope of the present invention is not limited to the following description. Accordingly, it should be clear that those skilled in the art can carry out an appropriate modification or variation to the following embodiments within the scope of the present invention, in view of the description of the present specification.

In one aspect, the present invention provides a method for identifying a stage of differentiation-maturation of a cell comprising the steps of: A) measuring an expression level of at least one cellular marker; and B) determining the stage of differentiation-maturation of the cell based on the expression level. Such expression level can be determined by using an immunological method such as flow cytometry or the like. Cellular markers used herein may be preferably markers related to differentiation/maturation stages of a stem cell of interest. It is understood that markers having such a relationship can be identified based on the disclosure of the present specification. Such identification methods include, for example, a method in which flow cytometry is conducted with respect to cellular markers in a stem cell, compared it with another indication which is already known to show differentiation stages (for example, a CD antigen described herein) to correlate the differentiation stage thereof and the cellular markers. Such stages may further be classified. CD antigens without differentiation relation, may preferably be avoided for use in such cells. Expression levels to be investigated may be expressed as a relative or absolute level.

Cellular markers used include preferably, at least two, more preferably at least three, still more preferably at least four, further preferably at least five cellular markers. It is understood that such multiple cellular markers may be used to investigate differentiation maturation stages in detail.

Such cellular markers include, but are not limited to, for example, CD1a, CD2, CD3, CD4, CD5, CD7, CD8, CD10, CD11b, CD13, CD14, CD15, CD16, CD19, CD20, CD22, CD23, CD33, CD34, CD45, TdT, FMC7 and HLA-DR, and the like. It should be understood that those skilled in the art can appropriately select such cellular markers depending on the type, lineage or the like of a cell to be analyzed (for example, a hematopoietic stem cell).

In one embodiment, stem cells of interest in the present invention include hematopoietic stem cells. Such typical stem cells include, but are not limited to, for example, monocytic lineage stem cells, B lymphocytic lineage stem cells, T lymphocytic lineage stem cells and myeloid lineage stem cells, and the like.

In another embodiment, the differentiation/maturation stage which may be analyzed by the present invention, includes Stage 1, Stage 2 and Stage 3 for monocytic lineage.

In another embodiment, the differentiation/maturation stage which may be analyzed by the present invention, includes Stage 1, Stage 2, Stage 3, Stage 4 and Stage 5 for myeloid lineage.

In another embodiment, the differentiation/maturation stage which may be analyzed by the present invention, includes Stage 1, Stage 2, Stage 3 and Stage 4 for B lymphocytic lineage.

In another embodiment, the differentiation/maturation stage which may be analyzed by the present invention, includes Stage 1, Stage 2, Stage 3 and Stage 4 for T lymphocytic lineage.

In a preferable embodiment, cellular markers used in the present invention include, but are not limited to, a combination of lineage CD antigens, and may include antigen combinations which are not lineage CD antigens. Such lineage CD antigens include as in the following Table.

TABLE 3

T lymphocytes: CD1a · b · c, CD2, CD2R, CD3, CD4, CD5, CD6,
CD7, CD8, CD27, CD28, CD29, CDw60, CD98, CD99, C99R,
CD100, CD152, CD153, CD154
B lymphocytes: CD10, CD19, CD20, CD21, CD22, CD23, CD24,
CD37, CD38, CD39, CD40, CD72, CD73, CD74, CDw75, CDw76,
CD77, CDw78, CD79a · b, CD80, CD81, CD82, CD83, CDw84,
CD85, CD86, CD138, CD139, CDw150
bone marrow cells/monocytes: CDw12, CD13, CD14, CD15,
CD15s, CD16a · b, CDw17, CD32, CD33, CD34, CD35, CD64,
CD65, CD65s, CD66, CD68, CD86, CD87, CD88, CD89, CD91,
CDw92, CD93, CD101, CD114, CD115, CD155, CD156, CD157,
CD163,
platelet: CD9, CDw17, CD31, CD36, CD41, CD42a · b · c · d,
CD63, CD107a · b, CD151
NK: CD16a · b, CD56, CD57, CD94, CD95, CD161
activated antigens: CD25, CD26, CD30, CD69, CD70, CD71,
CD96, CD97
endothelial cells: CD105, CD106, CDw109, CD140a · b, CD141,
CD142, CD143, CD144, CDw145, CD146, CD147
cell adhesion molecule: CD11a · b · c, CD18, CD29, CD29,
CD46a · b · c · d · e · f, CD50, CD51, CD54, CD56, CD58, CD62P,
CD62E, CD62L, CD102, CD103, CD104, CDw108, CD162, CD164,
CD165, CD166
cytokine receptors: CD25, CD116, CD117, CDw119, CD120a · b,
CD121a, CDw121b, CD122, CDw123, CD124, CD126, CD127,
CDw128, CD130, CD131, CD132, CD134, CD135, CDw136,
CDw137
non-lineage: CD43, CD44, CD44R, CD45, CD45RA, CD45RB,
CD45RO, CD46, CD47, CD48, CD52, CD53, CD55, CD59, CD148,
CDw149

FIGS. 1B through 1D depict the relationship between maturation and variation of expression of CD antigen, variation of surface antigens associated with differentiation of lymphocytes, and the relationship between differentiation of myeloid lineage cells and surface markers, respectively.

In a specific embodiment, the cellular markers used in the present invention for B lymphocytic lineage include combinations of: CD10, CD19, CD34 and CD45; CD10, CD20, CD5 and CD45; CD19, CD23, CD5 and CD45; CD10, CD20, CD34 and CD45; and CD5, CD19, CD34 and CD45, and the like.

In a specific embodiment, the cellular markers used in the present invention for T lymphocytic lineage include combinations of: CD1a, CD3, CD10 and CD45; CD4, CD8, CD7 and CD45; CD1a, CD3, CD34 and CD45; and CD4, Cd8, CD10 and CD45.

In a specific embodiment, the cellular markers used in the present invention for neutrophil lineage and monocytic lineage include combinations of: HLA-DR, CD11b, CD10 and CD45; CD16, CD13, CD33 and CD45; CD14, CD33, CD11b and CD45; CD4, CD10, CD34 and CD45; CD11b, CD13, CD16 and CD45; CD10, CD16, CD14 and CD45.

In another aspect, the present invention provides a system for identifying a differentiation/maturation stage of a cell. The subject system comprises A) means for measuring an expression level of at least one cellular marker; and B) means for determining the differentiation/maturation stage of the cell based on the expression level. Means for measurement may include for example, flow cytometry. Determination of differentiation/maturation stages may be conducted by performing a program stored on a CPU or the like, based on the expression level.

In the present invention, the means for measuring may measure a relative level of expression of a cellular marker.

In another embodiment, the means for measurement may include agents specific for the cellular marker used. Such agents may include, but are not limited to, for example, antibodies, and the like.

Cellular markers used in the present invention include at least two, preferably at least three, more preferably at least four, still more preferably at least five types thereof. The use of four cellular markers allows detailed classification which has not been conventionally achieved.

Cellular markers used in the system according to the present invention include, but are not limited to, for example, CD1a, CD2, CD3, CD4, CD5, CD7, CD8, CD10, CD11b, CD13, CD14, CD15, CD16, CD19, CD20, CD22, CD23, CD33, CD34, CD45, TdT, FMC7 and HLA-DR and the like.

In an embodiment of the system of the present invention, cells of interest of the present invention include hematopoietic cells, in particular, hematopoietic stem cells. Such exemplified cells include, but are not limited to, for example, monocytic lineage cells, B lymphocytic lineage cells, T lymphocytic lineage cells, myeloid lineage cells, and the like.

In another embodiment of the system of the present invention, the differentiation/maturation stage to be analyzed by the present invention includes Stage 1, Stage 2 and Stage 3 for monocytic lineage.

In another embodiment of the system of the present invention, the differentiation/maturation stage to be analyzed by the present invention includes Stage 1, Stage 2, Stage 3, Stage 4 and Stage 5 for myeloid lineage.

In another embodiment of the system of the present invention, the differentiation/maturation stage to be analyzed by the present invention includes Stage 1, Stage 2, Stage 3 and Stage 4 for B lymphocytic lineage.

In another embodiment of the system of the present invention, the differentiation/maturation stage to be analyzed by the present invention includes Stage 1, Stage 2, Stage 3 and Stage 4 for T lymphocytic lineage.

In a preferable embodiment of the system of the present invention, the cellular marker used in the present invention may comprise, but is not limited to, a combination of antigens which are lineage specific CD antigens, and may comprise a combination of antigens which are not lineage specific CD antigens.

In a specific embodiment of the system of the present invention, cellular markers used in the present invention for B lymphocytic lineage, include, but are not limited to: the combination of CD10, CD19, CD34 and CD45; the combination of CD10, CD20, CD5 and CD45; the combination of CD19, CD23, CD5 and CD45; the combination of CD10, CD20, CD34 and CD45; the combination of CD5, CD19, CD34 and CD45, and the like.

In a specific embodiment of the system of the present invention, cellular markers used in the present invention for T lymphocytic lineage, include, but are not limited to: the combination of CD1a, CD3, CD10 and CD45; the combination of CD4, CD8, CD7 and CD45; the combination of CD1a, CD3, CD34 and CD45; the combination of CD4, CD8, CD10 and CD45, and the like.

In a specific embodiment of the system of the present invention, cellular markers used in the present invention for myeloid lineage or monocytic lineage, include, but are not limited to: the combination of HLA-DR, CD11b, CD10 and CD45; the combination of CD16, CD13, CD33 and CD45; the combination of CD4, CD10, CD34 and CD45; the combination of CD11b, CD13, CD16 and CD45; the combination of CD10, CD4, CD14 and CD45, and the like.

In another aspect, the present invention provides a method for determining a differentiation/maturation stage of a cell such as a stem cell. The subject method includes the steps of A) providing a measurement pattern (“normal pattern”) with respect to a normal differentiation/measurement stage of the cell, as determined by cytometry of an expression level of at least one cellular marker; and B) determining the differentiation/maturation stage of the cell by comparing the pattern in flow cytometry of an expression level of the cellular marker compared with the normal pattern. Technologies used in the present method, may be any technologies described elsewhere herein. Exemplification of normal pattern include, for example, those normal patterns depicted in FIGS. 1R-E, IR-F and IR-G, further FIGS. 1R-H and IR-I show exemplifications of the normal Pathway. Characteristics of each cell and methods of identification thereof are described elsewhere herein and specifically in the section “Classification of Stage”. FIG. 1R-A shows graph comparing the conventional normal pathway and the normal pathway of the present invention in a bone marrow normal pathway.

In another aspect, the present invention provides a system for determining a differentiation/maturation stage of a cell such as a stem cell. The present system comprises: A) means for providing measurement pattern (“normal pattern”) with respect to a normal differentiation/measurement stage of the cell, as determined by cytometry of an expression level of at least one cellular marker; and B) means for determining the differentiation/maturation stage of the cell by comparing the pattern in flow cytometry of an expression level of the cellular marker compared with the normal pattern. Technologies used in the present system may be any technologies described elsewhere herein.

In another aspect, the present invention provides a method for determining whether or not the differentiation/maturation stage of a cell such as a stem cell is normal. The present method comprises A) measuring an expression level of at least one cellular marker by flow cytometry; and B) comparing a pattern of the expression level in flow cytometry, with an expression level of the cellular marker presented by a cell in a normal differentiation/maturation stage in flow cytometry, and establishing that there is difference therebetween, is indicative of an abnormal cell. Technologies used in the present method may be any technologies described elsewhere herein.

In another aspect, the present invention provides a system for determining whether a differentiation/maturation stage of a cell such as a stem cell is normal or not. The present system comprises: A) means for measuring an expression level of at least one cellular marker in a flow cytometry; and B) means for comparing a pattern of the expression level in a flow cytometry, with an expression level of the cellular marker presented by a cell in a normal differentiation/maturation stage in a flow cytometry, and the fact that there is difference therebetween is an indicative of comprising an abnormal cell. Technologies used in the present system may be any technologies described elsewhere herein.

In another aspect, the present invention provides a method for treating a subject based on a differentiation/maturation stage of a cell such as a stem cell. The present method comprises the steps of: A) measuring an expression level of at least one cellular marker; B) determining the differentiation/maturation stage of the cell based on the expression level; and C) providing the subject with an appropriate treatment for the determined differentiation/maturation stage. Technologies used in the present method may be any technologies described elsewhere herein.

In another aspect, the present invention provides a method for treating a subject based on a differentiation/maturation stage of a cell such as a stem cell. The present method comprises the steps of: A) measuring an expression level of at least one cellular marker; B) determining the differentiation/maturation stage of the cell based on the expression level; and C) providing the subject with an appropriate treatment based on the result of comparing the determined differentiation/maturation stage, and a stage which the cell takes in its normal stage. Technologies used in the present method may be any technologies described elsewhere herein.

In a preferable embodiment, an appropriate treatment includes, but is not limited to, i) continuing the present therapy or termination of the present therapy and to conduct follow-up only, when normal cells exists or the cells present are determined to be normal immature cells with respect to the determined differentiation/maturation stage of the cell; and ii) enhancement or alteration of the present therapy when the cells present are abnormal cells with respect to the determined differentiation/maturation stage of the cells, and the like.

In another embodiment, the treatment applied in the present invention comprises conducting an additional stem cell transplantation when depletion of the original stem cells or an abnormality of differentiation/maturation progress is recognized, even if the existing cells are normal immature cells with respect to the determined differentiation/maturation stage of the cells.

In another aspect, the present invention provides a system for treating a subject based on differentiation/maturation stage of a cell. This system comprises A) means for measuring an expression level of at least one cellular marker; B) means for determining the differentiation/maturation stage of the cell based on the expression level; and C) means for providing to the subject an appropriate treatment for the determined differentiation/maturation stage. In this system, any technologies as described hereinabove may be applied and used for specific embodiments thereof.

In another aspect, the present invention provides a system for providing a subject with an appropriate treatment based on a differentiation/maturation stage of a cell. This system comprises A) means for measuring an expression level of at least one cellular marker; B) means for determining the differentiation/maturation stage of the cell based on the expression level; and C) means for providing the subject with an appropriate treatment based on the determined differentiation/maturation stage. In this system, any technologies as described hereinabove may be applied and used for specific embodiments thereof.

In another aspect, the present invention provides a system for treating a subject based on a differentiation/maturation stage of a cell, for example, after treatment of leukemia. This system comprises A) means for measuring an expression level of at least one cellular marker; B) means for determining the differentiation/maturation stage of the cell based on the expression level; and C) means for providing to the subject an appropriate treatment based on the result of comparing the determined differentiation/maturation stage, and a stage which the cell takes in its normal stage. In this system, any technologies as described hereinabove may be applied and used for specific embodiments thereof.

In another aspect, the present invention provides a system for providing a subject with an appropriate treatment based on a differentiation/maturation stage of a cell, for example, after treatment of leukemia. This system comprises A) means for measuring an expression level of at least one cellular marker; B) means for determining the differentiation/maturation stage of the cell based on the expression level; and C) means for providing the subject with an appropriate treatment based on the result of comparing the determined differentiation/maturation stage, and a stage which the cell takes in its normal stage. In this system, any technologies as described hereinabove may be applied and used for specific embodiments thereof.

Other aspects of the present invention provides a pattern of differentiation/maturation of a cell produced by the present method, and a storage medium, transmission medium and the like, with such a pattern stored thereon.

All scientific publications, patents, patent applications and the like cited herein are incorporated by reference in their entireties, as if set forth fully herein.

The present invention has heretofore been described by way of preferred embodiments for better understanding of the present invention. Hereinafter, the present invention will be described by way of examples. The examples described below are provided only for illustrative purposes and are not intended to limit the present invention. Accordingly, the scope of the present invention is not limited by the embodiments and examples specified herein except as by the appended claims.

### EXAMPLES

The present invention will be described in greater detail by way of examples. The present invention is not limited to the examples below. Reagents and the like used in the following Examples are obtained from Sigma (St. Louis, USA), Wako Pure Chemicals (Osaka, Japan) and the like, unless otherwise stated. Animals were treated in accordance with the rules defined by the Ministry of Health, Labor and Welfare.

### Example 1

### Analysis of Immature Cell Surface Antigens in Normal Bone Marrow Blood

(Methods)

1. Target

After informed consent was obtained, bone marrow blood obtained from a patient was used for analysis of surface antigen of immature cells in normal bone marrow blood. After obtaining bone marrow blood using heparin, cells were suspended in culture medium (RPMI-1640) supplemented with 10% FCS and stored at 4° C. Cell surface antigens were analyzed within 48 hours.

2. Monoclonal Antibody

Fluorescein isothiocyanate, FITC, labeled antibodies against the following CD were obtained from Beckton Dickinson Immunocytometry Systems (BDIS: San Jose, Calif.); anti-CD5, CD7, CD8, CD14, CD19, CD20, and HLA-DR antibody.

Phycoerythrin, PE, labeled antibodies against the following CD, were obtained from Beckton Dickinson Immunocytometry Systems (BDIS: San Jose, Calif.); mouse IgG2a anti-CD4, CD8, CD11b, CD13, CD14, CD19, CD33, and CD34 antibody.

Peridinin chlorophyll protein, PerCP, labeled antibodies against the following CD, were obtained from Beckton Dickinson Immunocytometry Systems (BDIS: San Jose, Calif.); anti-CD45 antibody.

### Allophycocyanin, APC, labeled antibodies against the following CD were obtained from Becton Dickinson Immunocytometry Systems (BDIS: San Jose, Calif.); mouse IgG2a anti-CD4, CD10, and CD33 antibody.

FITC labeled antibodies against the following CD, were obtained from BD Bioscienses-Permingen (PerMingen: San Diego, Calif.); mouse IgG1 anti-CD1a, CD16 and CD34 antibody.

APC labeled antibodies against the following CD were obtained from BD Bioscienses-Permingen (PerMingen: San Diego, Calif.); anti-CD3, CD5, CD11b and CD34 antibody.

FITC labeled antibodies against the following CD, were obtained from DakoCytomation (DaKo: DK-2600, Glostrup); FITC labeled anti-CD23 antibody and PE labeled anti-CD34 antibody.

3. Staining and Measurement

The concentration of leukocytes in a subject sample was measured, and a solution containing 2-5×10⁶ cells/ml of leukocytes was prepared using phosphate buffered saline (PBS: 0.1% BSA, 0.1% sodium azide). To each solution in a tube, an antibody was added, then sample of 100 μl whose concentration was adjusted was collected and mixed well and incubated at room temperature for 20 minutes under dark condition. Then a hemolytic agent (8.26 g NH₄Cl, 1.00 g KHCO₃, EDTA-4Na 0.037 g/L in distilled water) was added, and incubated at room temperature for 5 minutes to hemolyse contaminating erythrocytes. The hemolysed solution was washed with PBS and fixed with 1% paraformaldehyde (CellFIX: BDIS).

4. Measurement Method

Measurement was carried out using FACSCalibur-4A (BDIS). In setting up the apparatus, Lyse-Wash mode of FACSComp software was used with CaliBRITE beads (BDIS). Stability of the apparatus was confirmed by using SPHERO™ Rainbow Calibration Particles (RCP: PerMingen). Uptake of cells was carried out using CellQuest software. Ten thousand cells were measured and stored as List Mode Data.

5. Analysis of Expression Intensity of Cell Surface Antigen in Blood of a Normal Bone Marrow.

Positions for each stage cell were identified based on Stored List Mode Data by using WinList (Verity Software House, Inc.) software Version 3.0, with setting arbitrary parameter and developing into Dot-Plot. Detailed parameter and gate setting were as follows:

Parameters to be used were forward scatter (FSC) reflecting size of a cell, side scatter (SSC) reflecting the structure of a cell, FL-1 reflecting the reaction of an FITC labeled antibody, FL-2 reflecting the reaction of a PE labeled antibody, FL-3 reflecting the reaction of a PerCP labeled antibody, and FL-4 reflecting the reaction of an APC labeled antibody.

Combinations of each parameter were FSC-FL-1, FSC-FL-2, FSC-FL-3, FSC-FL-4, SSC-FL-1, SSC-FL-2, SSC-FL-3, SSC-FL-4, FL-3-FL-1, FL-3-FL-2, FL-3-FL-4, FL-1-FL-2, FL-4-FL2, FL4-FL-1, and FSC-SSC. Gate setting was set at a position according to a method of identification of each type of cell as described on Page 34. A cell satisfying all gate conditions were extracted.

(Result)

FIG. 3 shows the change in each CD antigen expression level of each stage for each stem cell of B lymphocytic lineage, T lymphocytic lineage, neutrophil lineage, and monocytic lineage.

As shown in FIG. 2, it is understood that at least the following CD antigens can be used for classification of differentiation maturation stages for hematopoietic stem cells of each lineage and progenitor thereof:

### B Lymphocytic Lineage

TdT, CD34, CD10, CD45, CD19, CD20, CD22, CD5, FMC7, CD5, and CD23,

### T Lymphocytic Lineage

CD34, CD1a, CD5, CD45, CD10, CD3, CD7, CD4, CD8, and CD2

### Myelocyte Lineage

CD34, CD15, HLA-DR, CD11b, CD13, CD33, CD16, and CD45

### Monocytic Lineage

CD33, CD34, CD13, CD11b, CD15, HLA-DR, CD45, and CD14

As clearly understood based on FIG. 2, for example, in observing the expression level of antigen, e.g. CD10, for the B lymphocytic lineage, observation of only one antigen allow classification of a cell into all stages. On the other hand, it can be understood that, regarding CD antigens which behave in a same manner in Stage 2 and Stage 3, such as CD15 and HLA-DR of the monocytic lineages, observation of only 2 antigens is not sufficient, and it is necessary to observe CD antigens, e.g. CD14, which can distinguish between Stage 2 and Stage 3. In summary, differentiation between different maturation stages, as shown in FIG. 1, can be understood.

### Example 2

### Observation of Myelocyte

Then, regarding a normal myelocyte lineage cell and a myelocyte lineage cell of Myelodyspolastic Syndrome, observation was carried out as shown in Example 1 with using CD13 (PE labeled) and CD16 (FITC labeled). Normal Pathway was made by using dot plot.

Results are shown in FIG. 3. In FIG. 3, pathways for normal myelocytes (left panel) and bone marrow dysplasia syndrome (right panel) are shown. As shown in left panel, when using normal myelocytes, a constant Pathway was always found. Whereas, in using a myelocyte lineage cell of a bone marrow dysplasia syndrome patient, a pathway different from the normal pathway was observed. Accordingly, it can be understood that the myelocyte lineage cell of the bone marrow dysplasia syndrome patient contains only abnormal cells.

### Example 3

Then, a similar experiment was carried out and normal pathways for B lymphocytic lineage, T lymphocytic lineage, myelocyte lineage, and monocytic lineage at mature stage were identified.

According to a method described in Example 1, CD10 (PE labeled) and CD20 (FITC labeled) were used for B lymphocytic lineage, CD4 (PE labeled) and CD8 (FITC labeled) were used for T lymphocytic lineage, and CD13 (PE labeled) and CD16 (FITC labeled) were used for myelocyte lineage and monocytic lineage.

Results are shown in FIG. 4. Normal pathways, as indicated by arrows in the figure, were identified.

By repeating the same experiments, a typical normal Pathway analysis panel as follows was identified:

B Lymphocytic Lineage
CD20×CD10×CD34×CD45
CD5×CD19×CD34×CD45
CD10×CD19×CD34×CD45
CD10×CD20×CD5×CD45
CD19×CD23×CD5×CD45

T Lymphocytic Lineage
CD1a×CD3×CD10×CD45
CD1a×CD3×CD34×CD45
CD4×CD8×CD10×CD45
CD4×CD8×CD7×CD45

Neutrophil/Monocytic Lineage.
HLA-DR×CD11b×CD10×CD45
CD16×CD13×CD33×CD45
CD16×CD13×CD11b×CD45
CD16×CD14×CD10×CD45
CD14×CD33×CD11b×CD45
CD4×CD10×CD34×CD45.

### Example 5

According to a method described in Example 1, a patient was diagnosed and appropriately provided with treatment.

### Name of a Patient: Y. S., Acute Leukemia

Dec. 2, 2003 Ja-0028 (Diagnosis at recurrence): 1.2% lymphocyte, 0.0% monocyte, 2.6% bone marrow form, and 94.4% abnormal lymphoblast. Acute leukemia cells were observed as 94.4% of whole leukocyte.

Dec. 17, 2003 Ja-0034 (when two cycles of remission induction treatment completed): 73.9% lymphocyte, 1.1% monocyte, 7.4% bone marrow form, and 8.6% abnormal lymphoblast. Acute leukemia cells were observed as 8.6% of whole leukocyte.

Jan. 5, 2004 Ja-0041 (after remission induction): 2.8% lymphocyte, 1.6% monocyte, 92.7% bone marrow form, 0.1% lymphoblast, and 0.4% myeloblast. Acute leukemia cells were not observed. Myeloblast showed normal differentiation-maturation pattern. Many matured myeloid cells was observed. Whereas, there were less immature myeloid cells. Acute bone marrow remodeling was observed.

Feb. 3, 2004 Ja-0052 (one week after transplantation of peripheral blood stem cells): 0.6% lymphocyte, 3.1% monocyte, 95.2% bone marrow form, 0.1% lymphoblast, and 0.5% myeloblast. Acute leukemia cells were not observed. Myeloblast showed normal differentiation-maturation pattern.

Feb. 8, 2004 Ja-0069 (after transplantation of peripheral blood stem cells, observing progress): 6.0% lymphocyte, 5.9% monocyte, 61.9% bone marrow form, 14.6% lymphoblast, and 3.1% myeloblast. Though it could not be confirmed in detail, due to trace amount, a dot plot of 2˜3/10,000, which is similar to acute leukemia cells, was observed. No abnormality was observed for lymphoblast cells and myeloblast, however, some turbulence in myeloid cell pathway were observed, which was recognized as forerunner of recurrence.

Apr. 19, 2004 Ja-0095 (observing progress): 2.5% lymphocyte, 5.5% monocyte, 66.6% bone marrow form, 13.6% lymphoblast, and 2.9% myeloblast. Acute leukemia cells were observed as 0.7% of whole leukocyte. Presage of recurrence was observed. Treatment to maintain and enhance remission is required.

May 24, 2004 Ja-0111 (Second time; starting pretreatment for allogenic bone marrow transplantation):10.0% lymphocyte, 5.9% monocyte, 62.0% bone marrow form, 8.6% lymphoblast, and 1.6% myeloblast. Acute leukemia cells were observed as 0.06% of whole leukocyte.

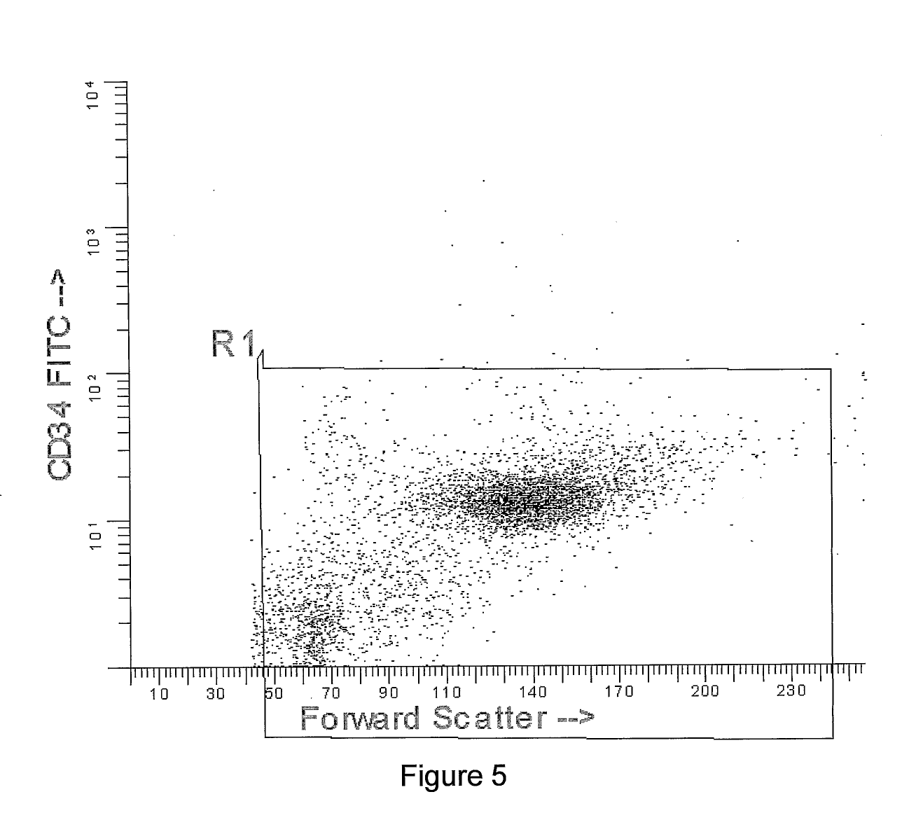

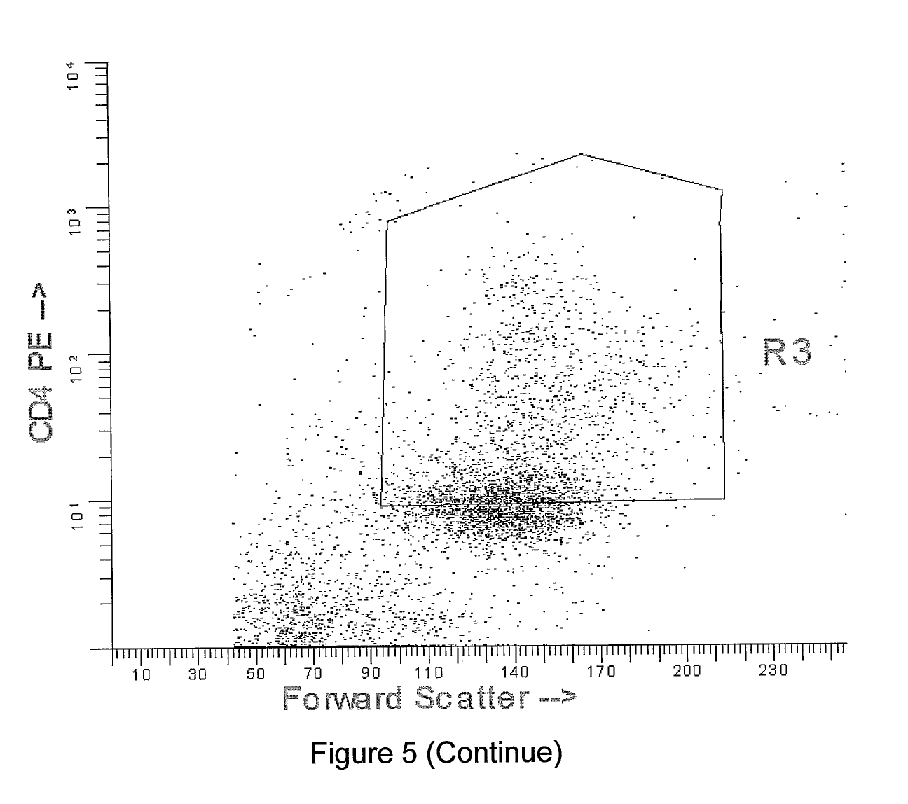

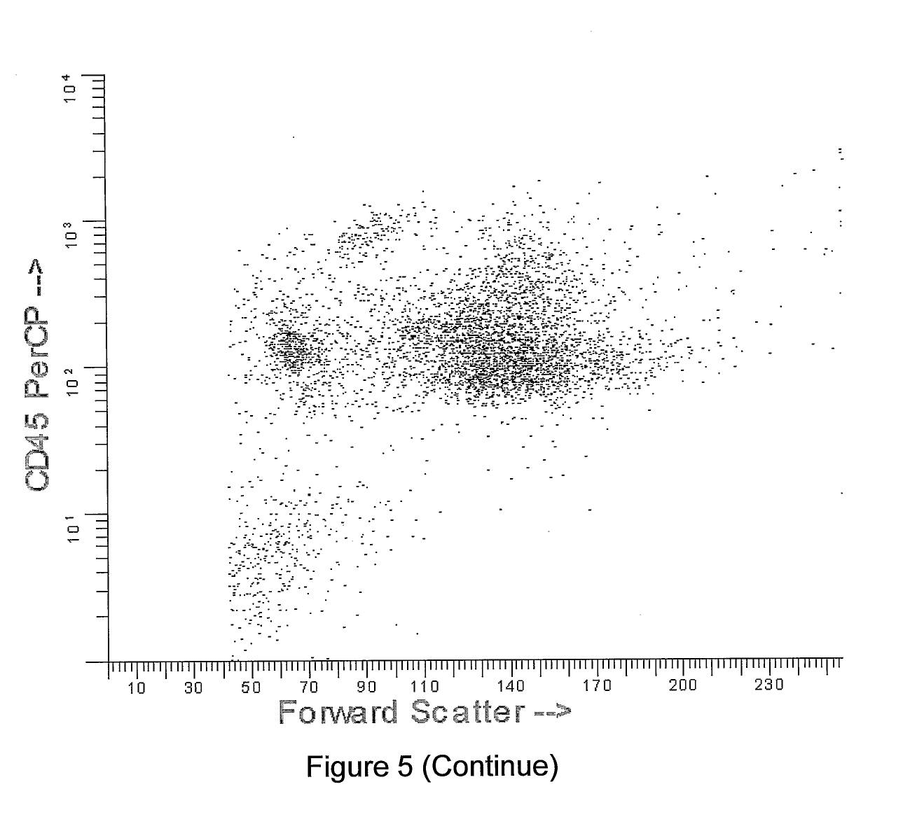

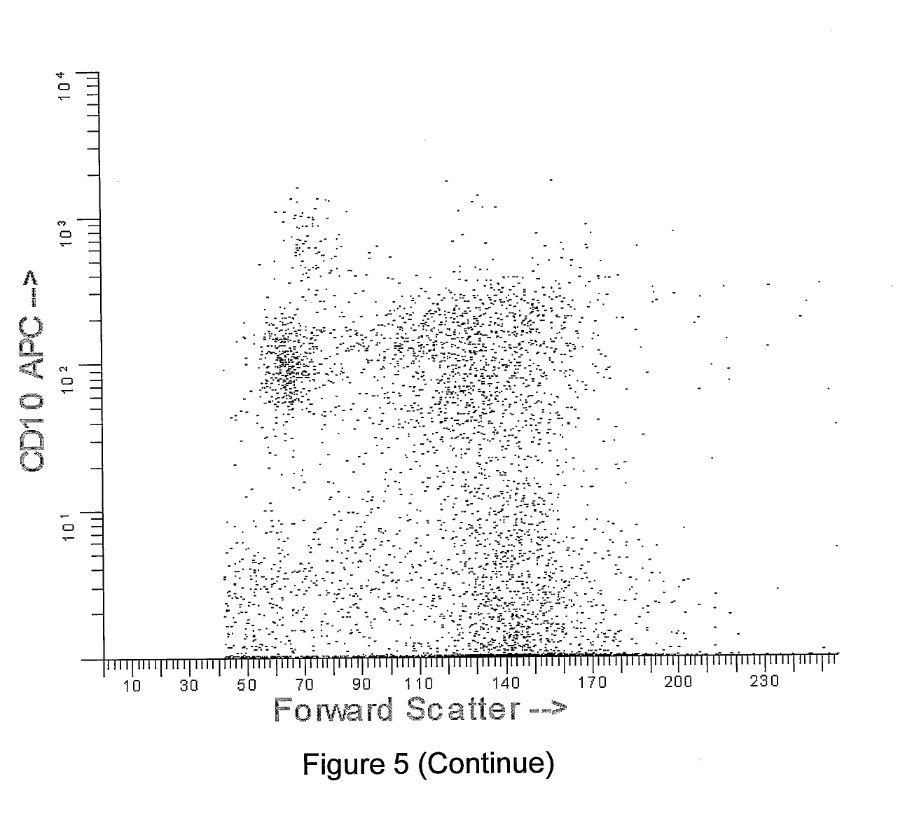

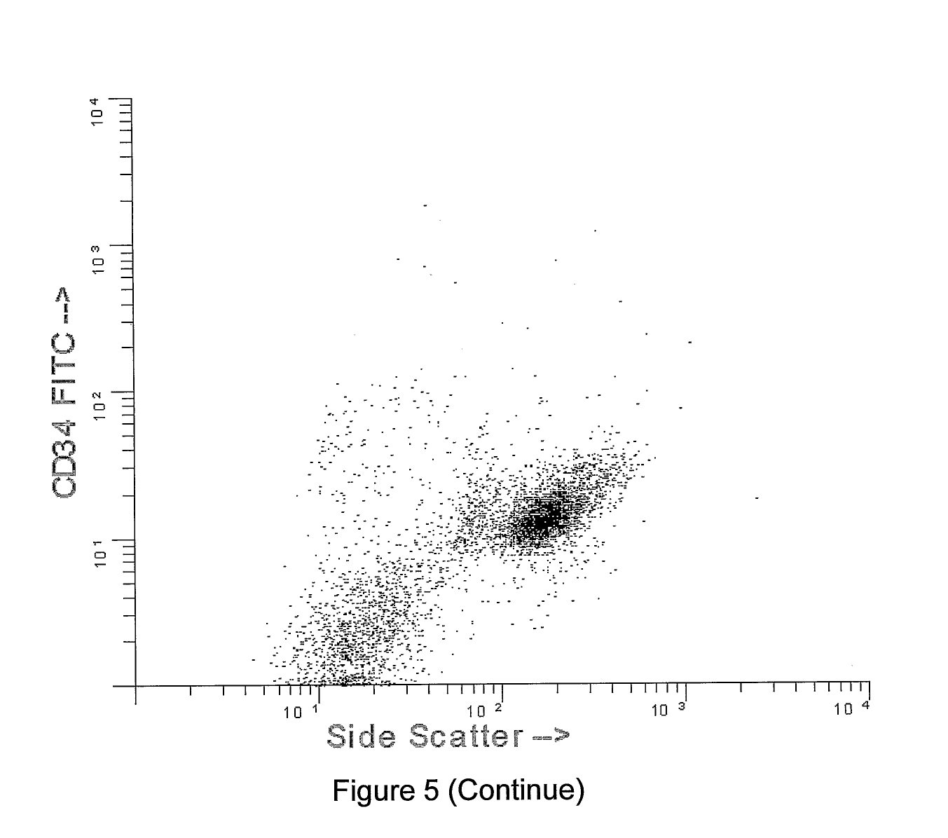

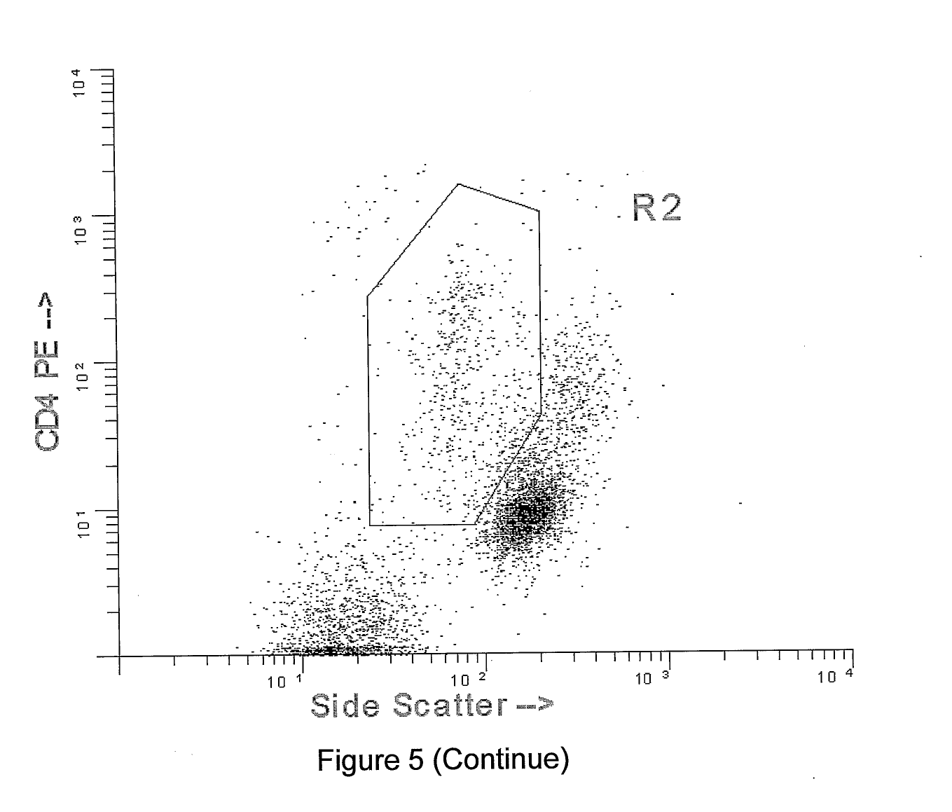

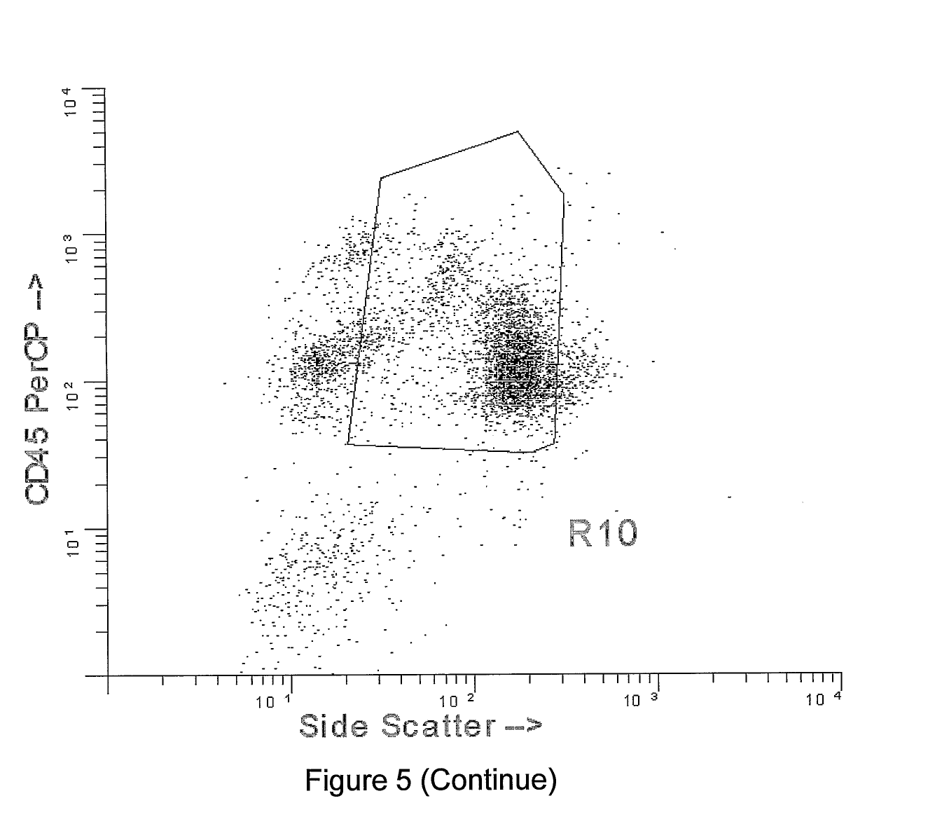

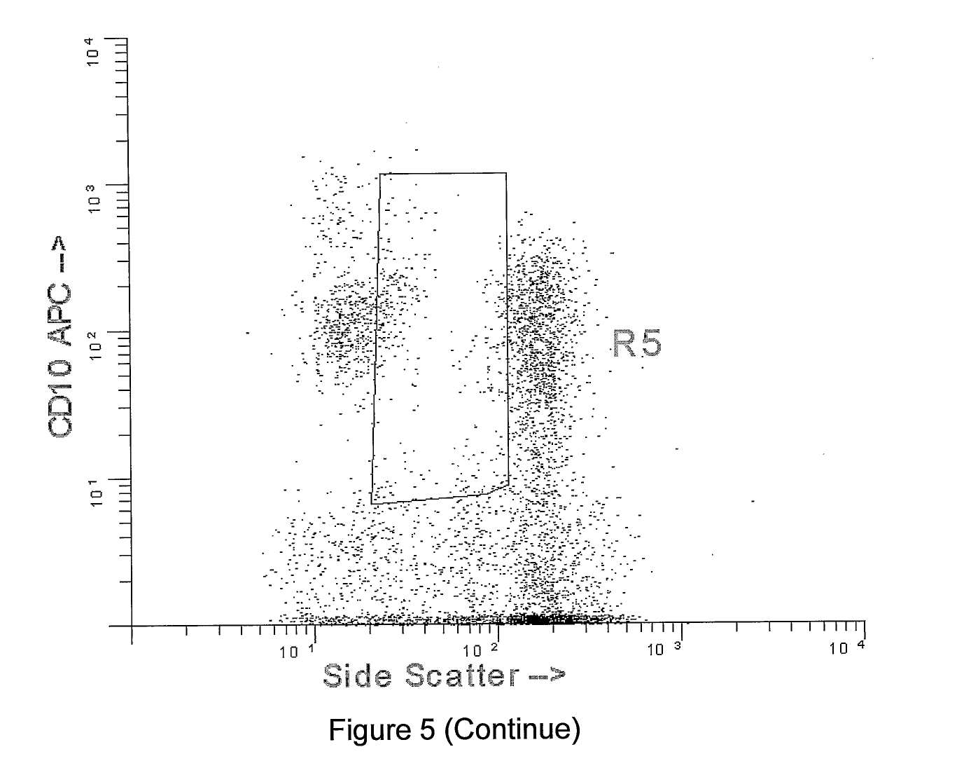

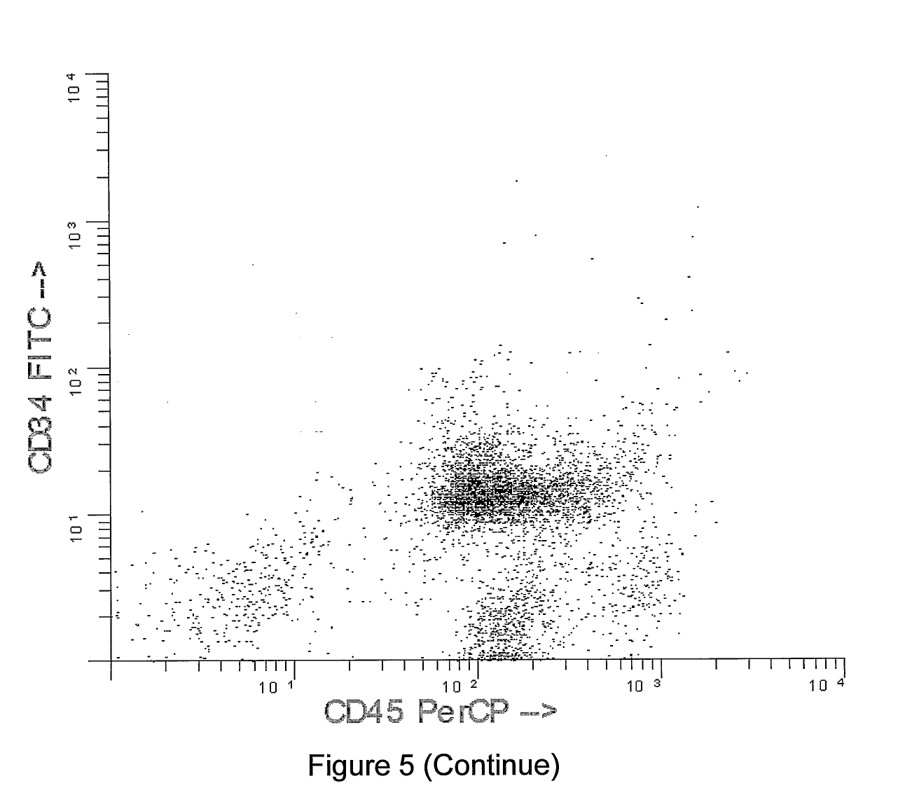

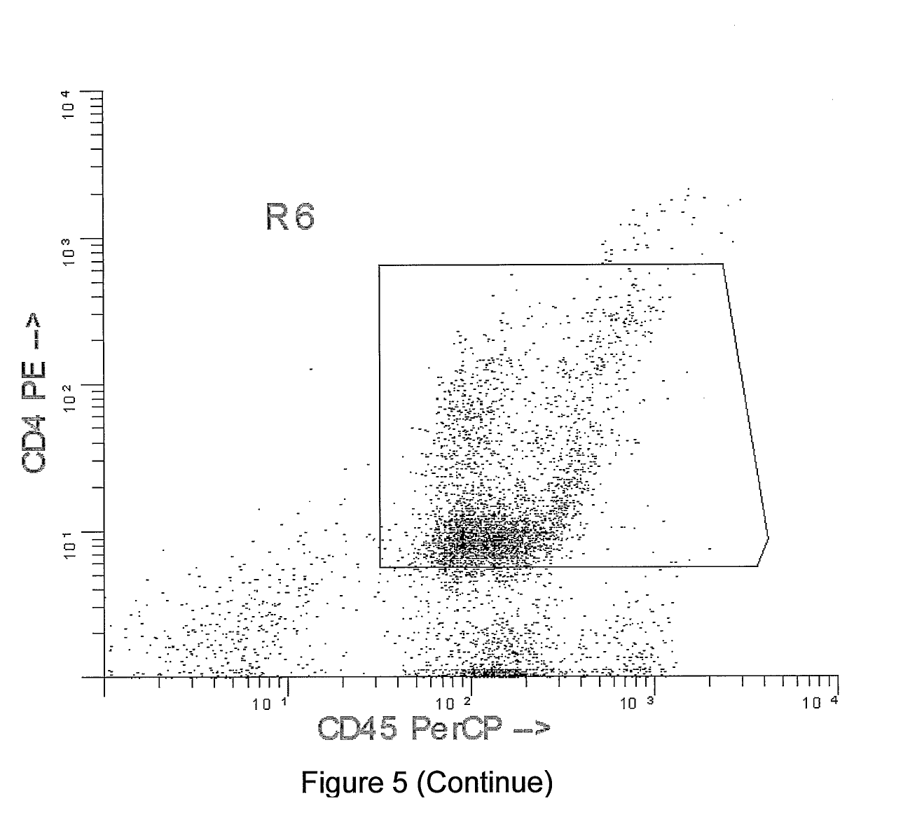

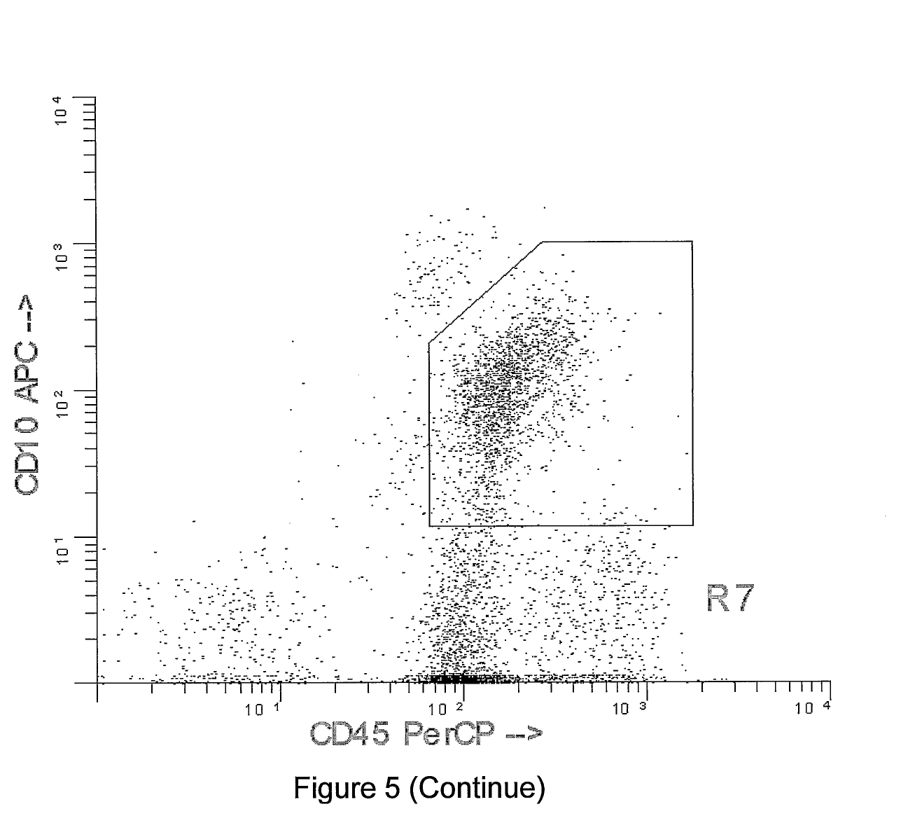

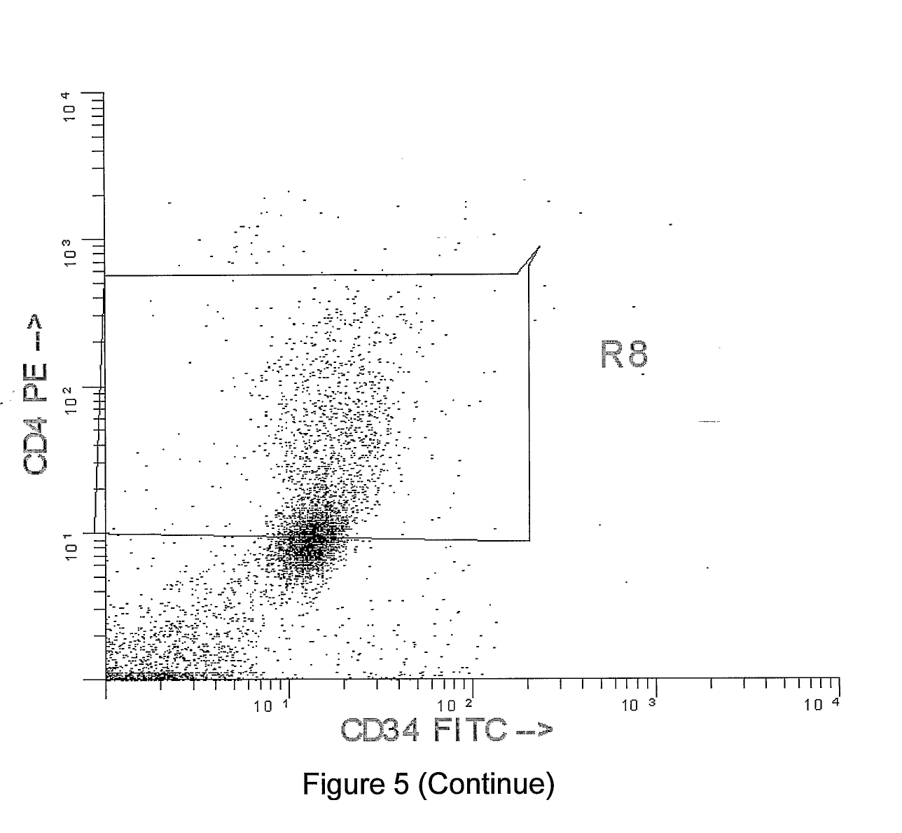

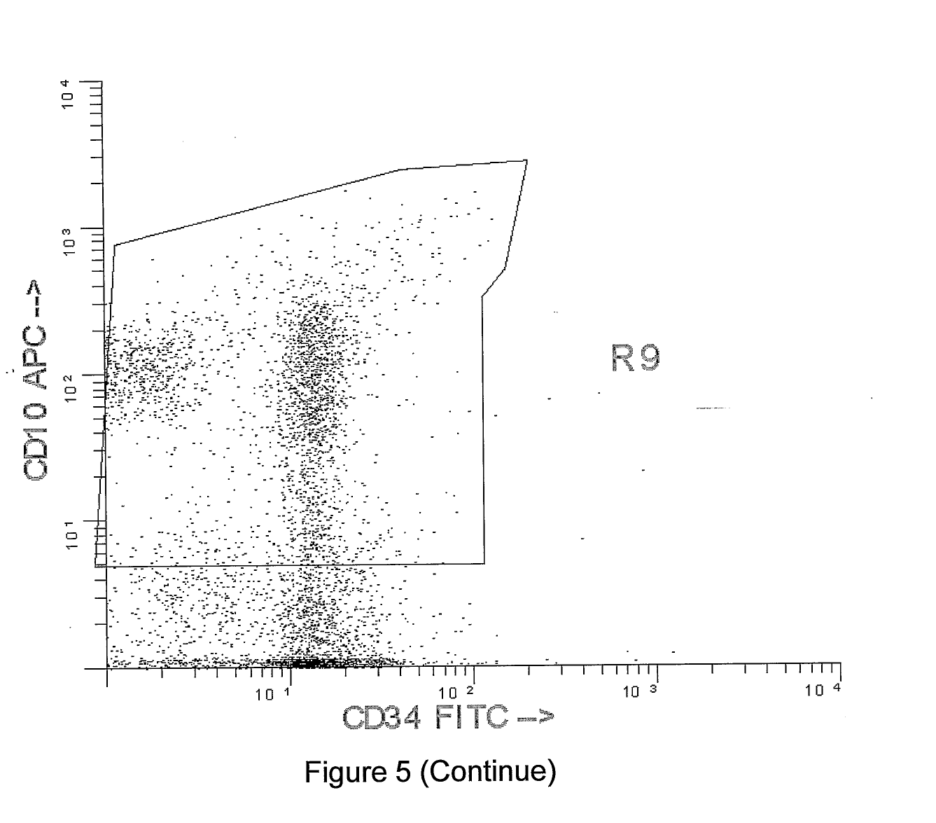

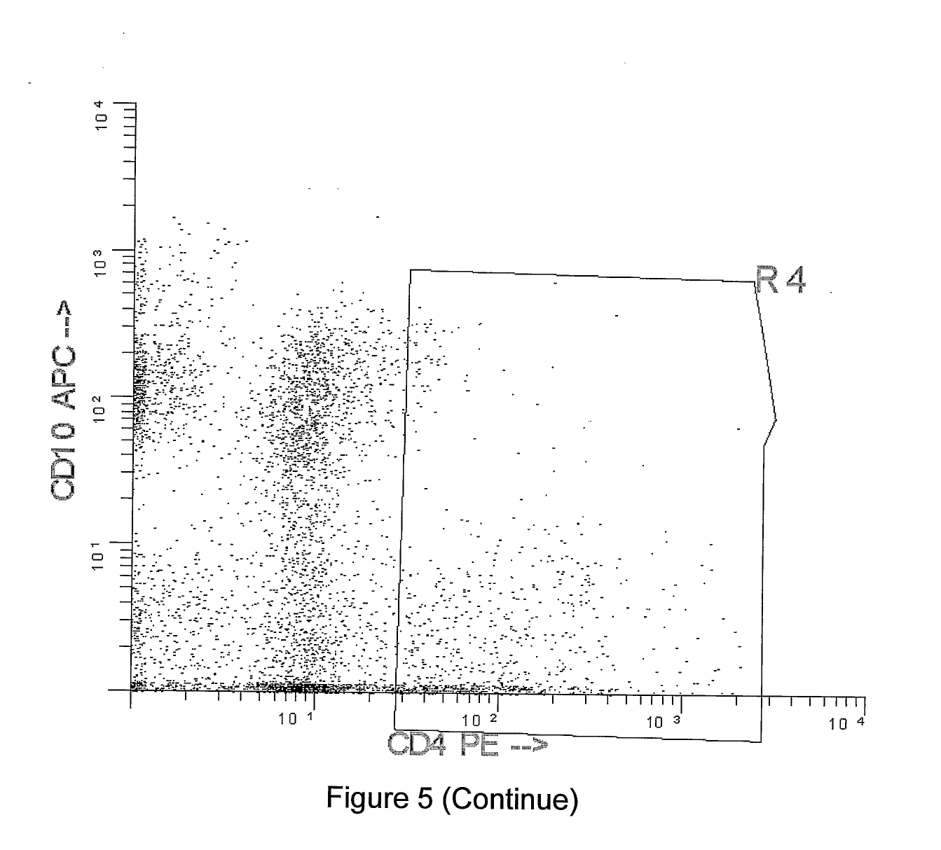

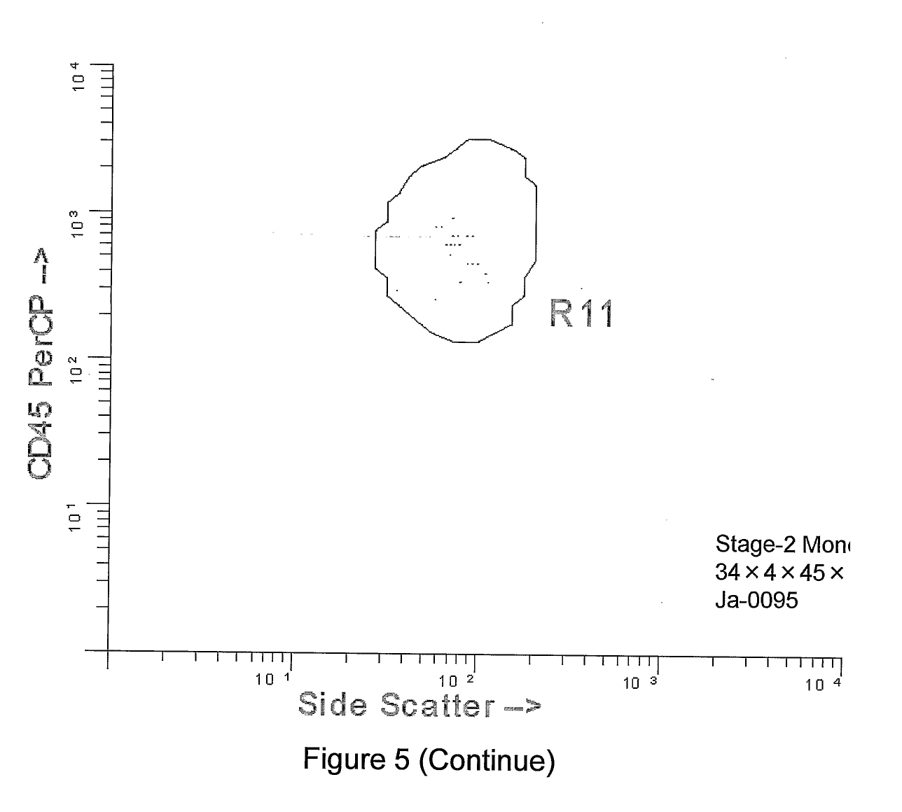

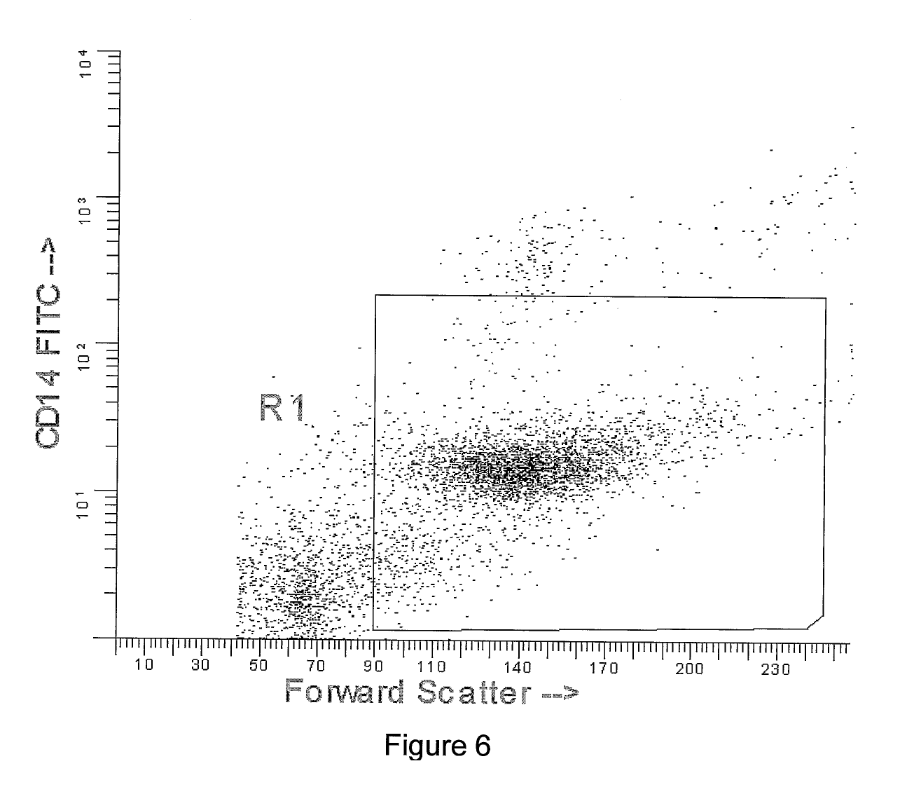

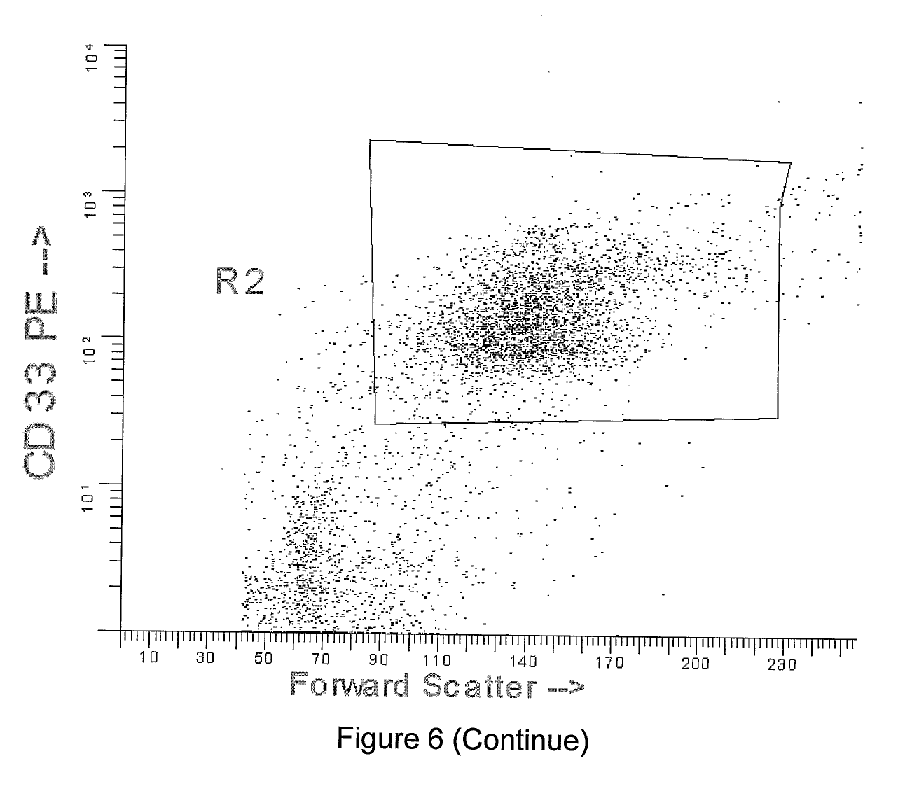

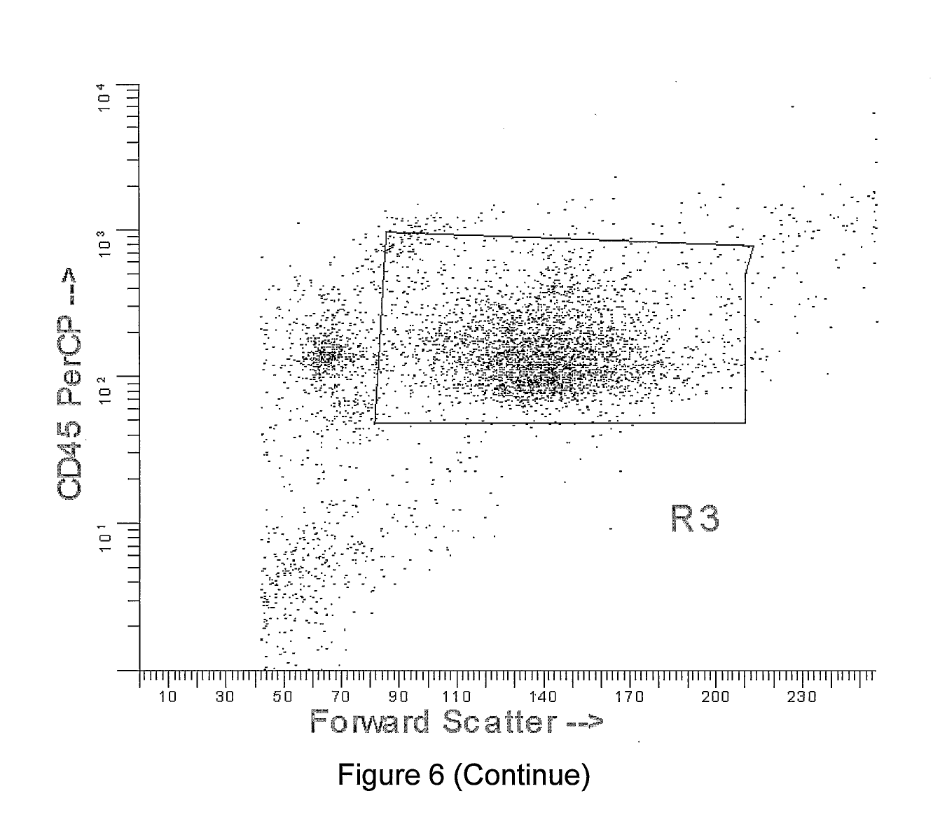

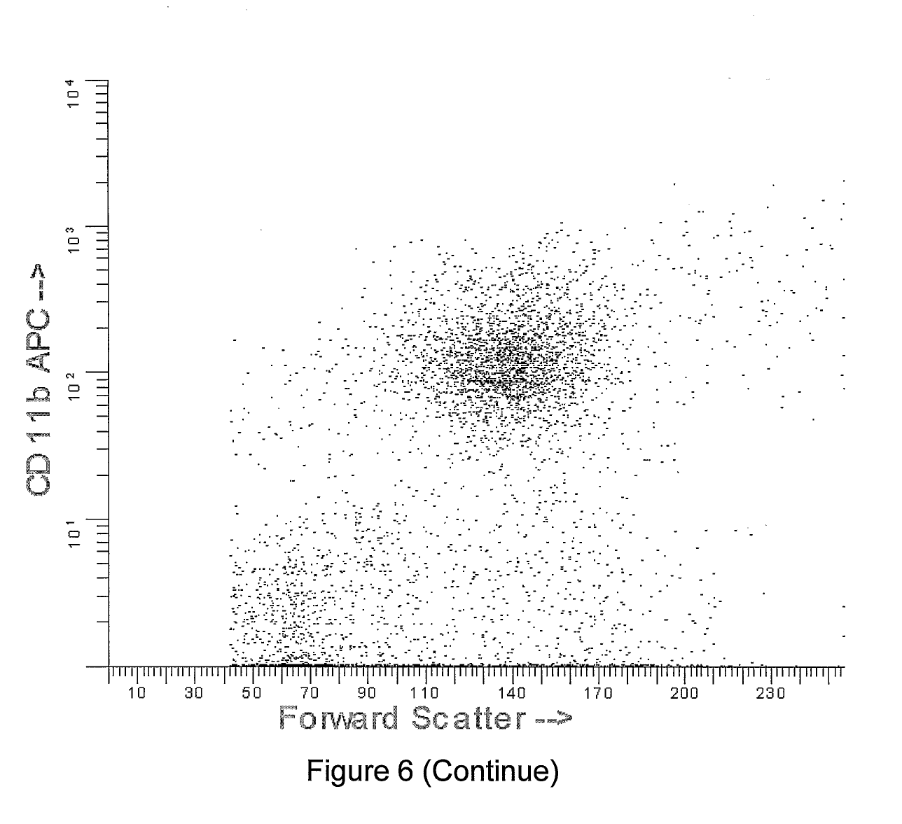

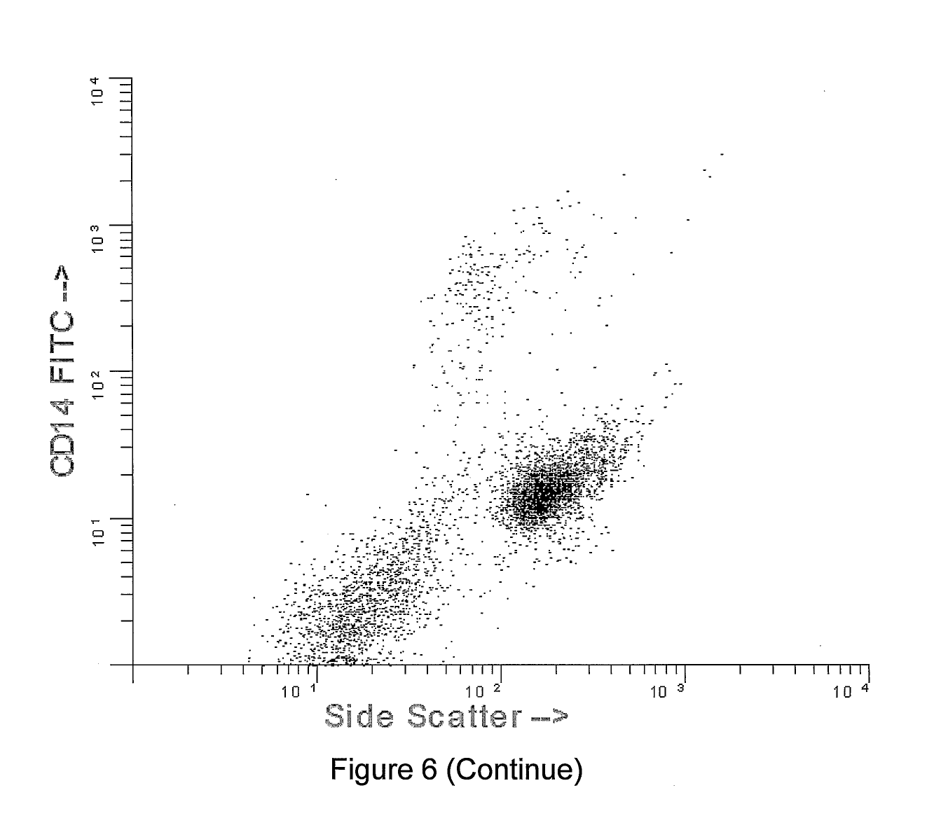

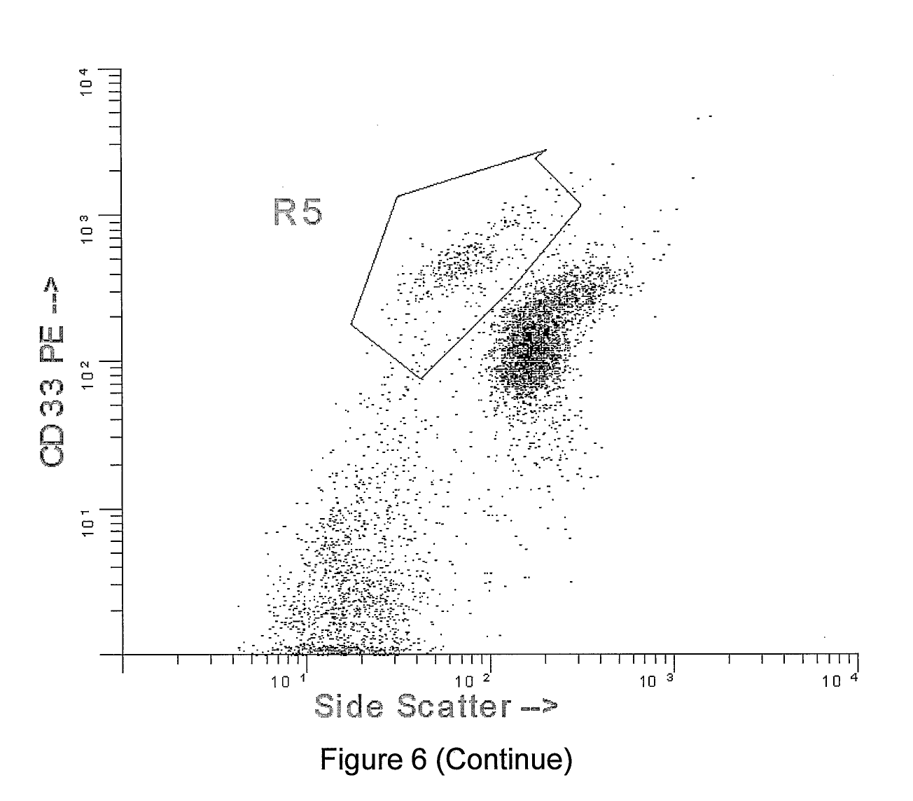

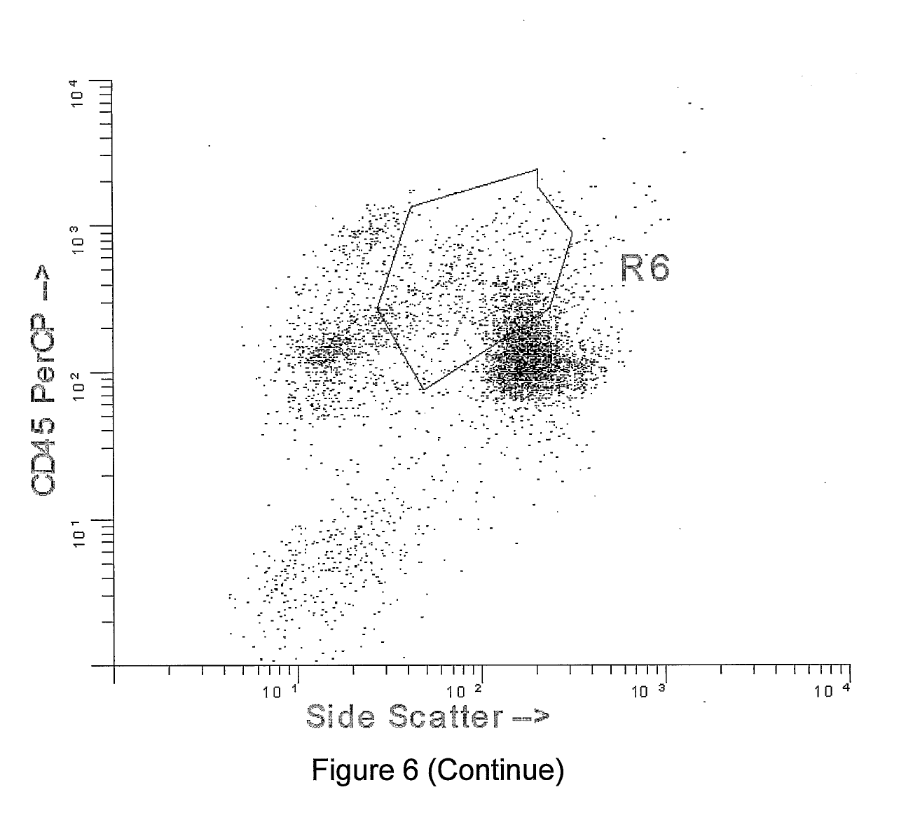

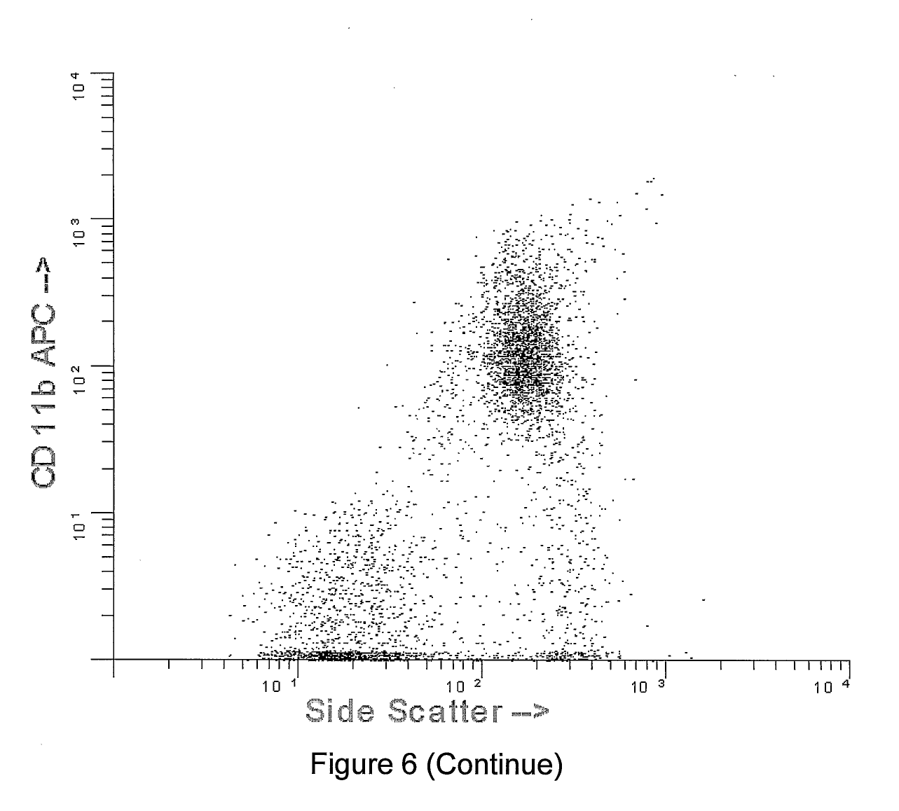

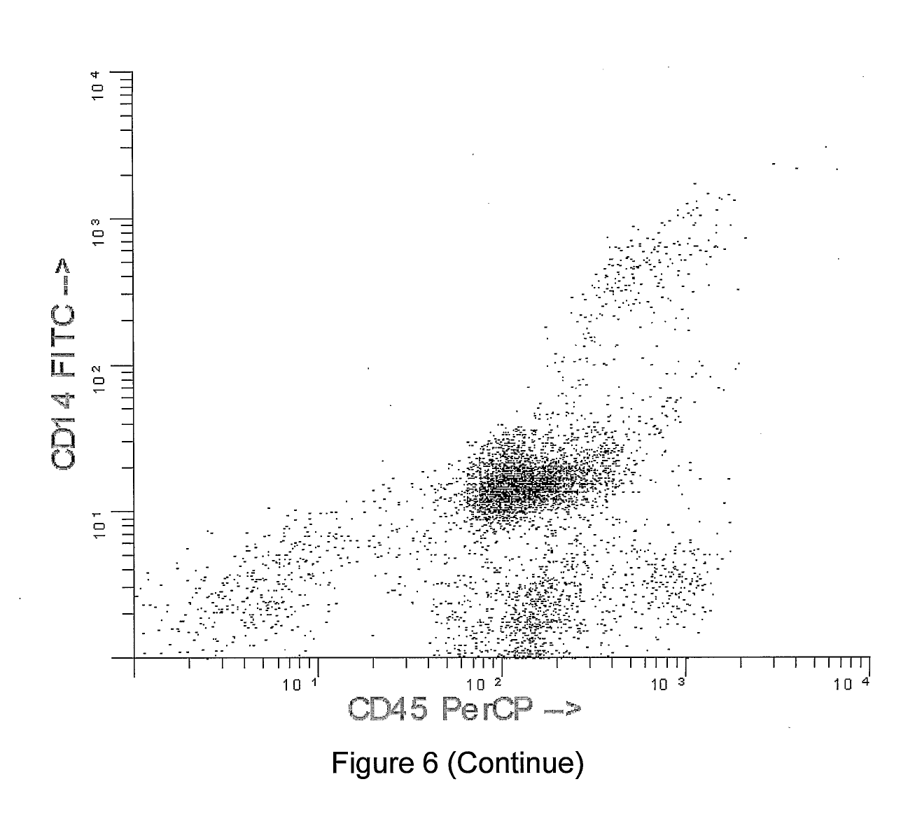

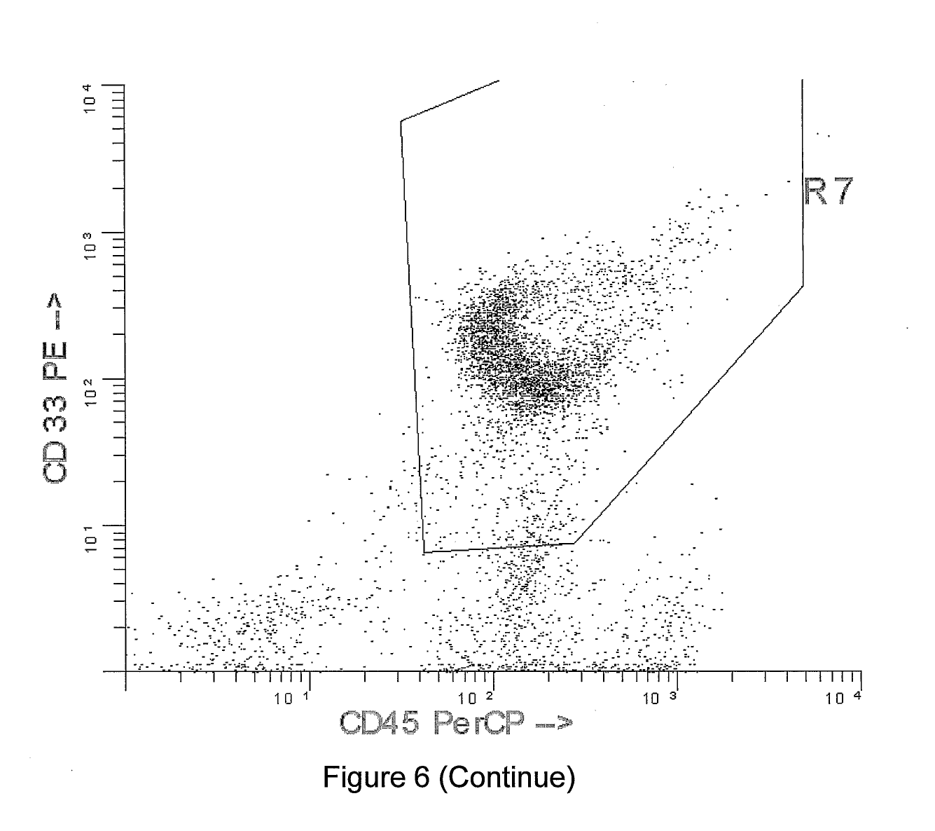

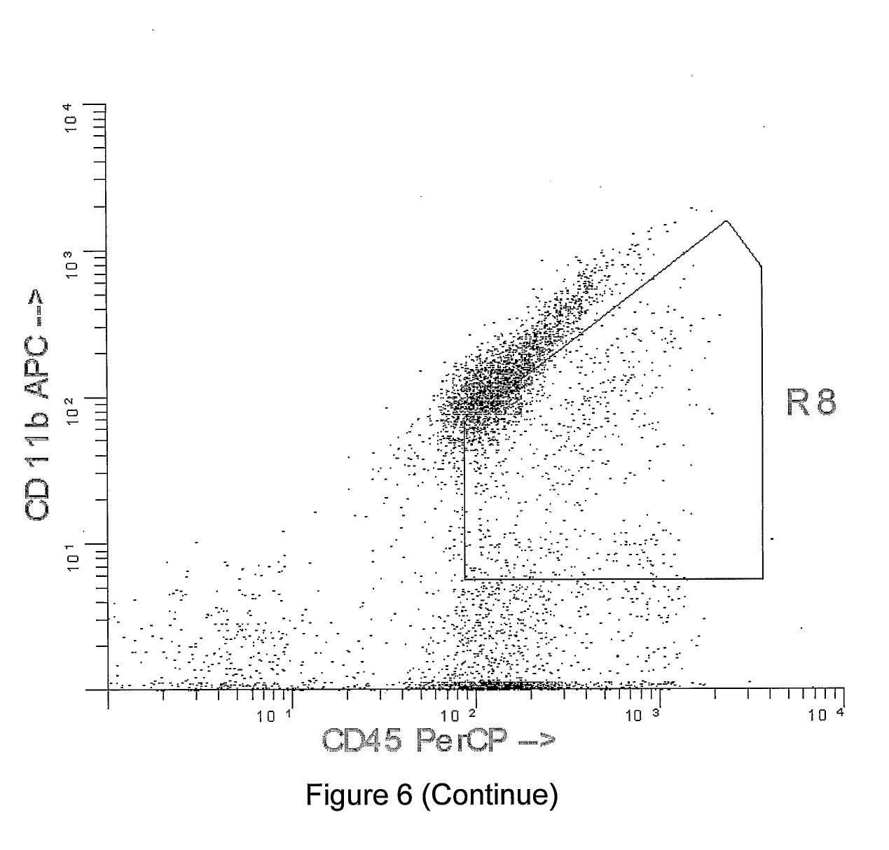

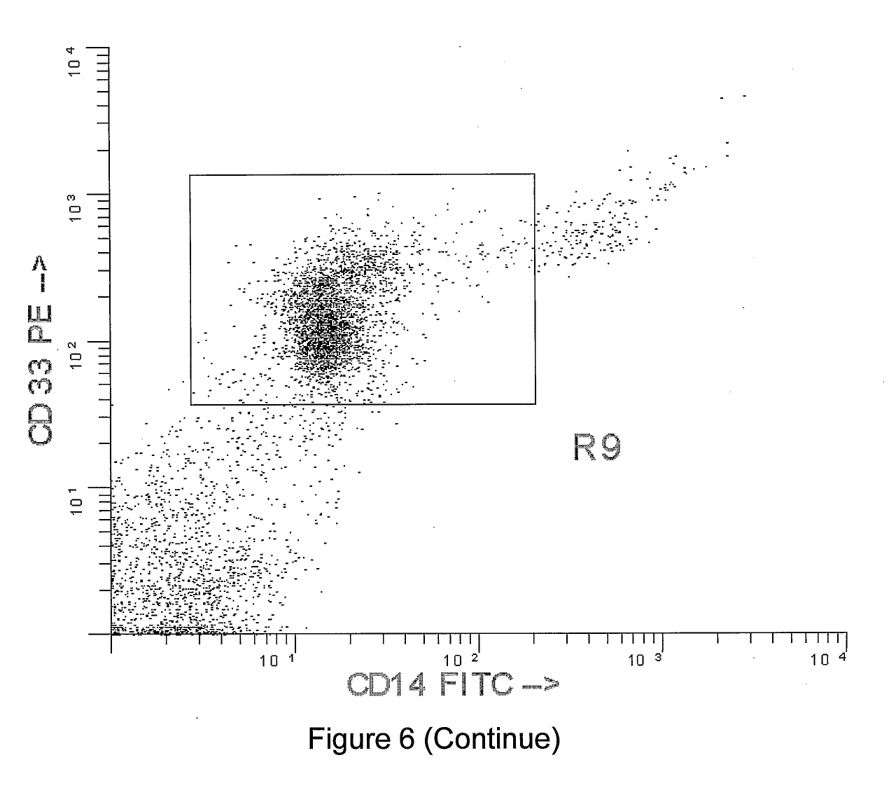

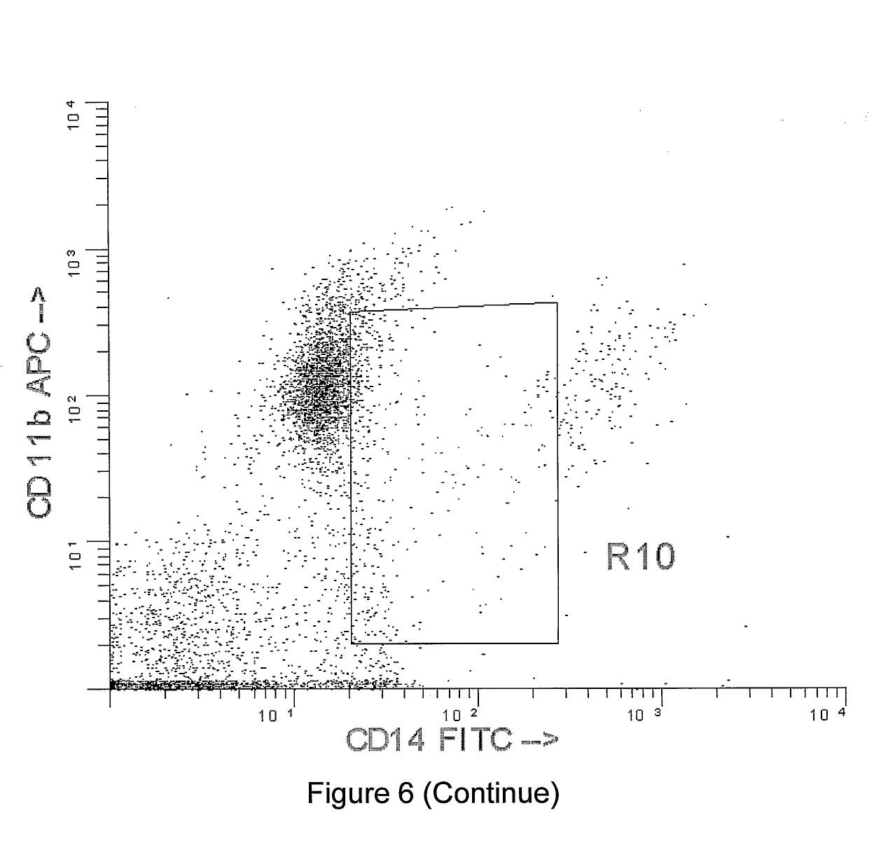

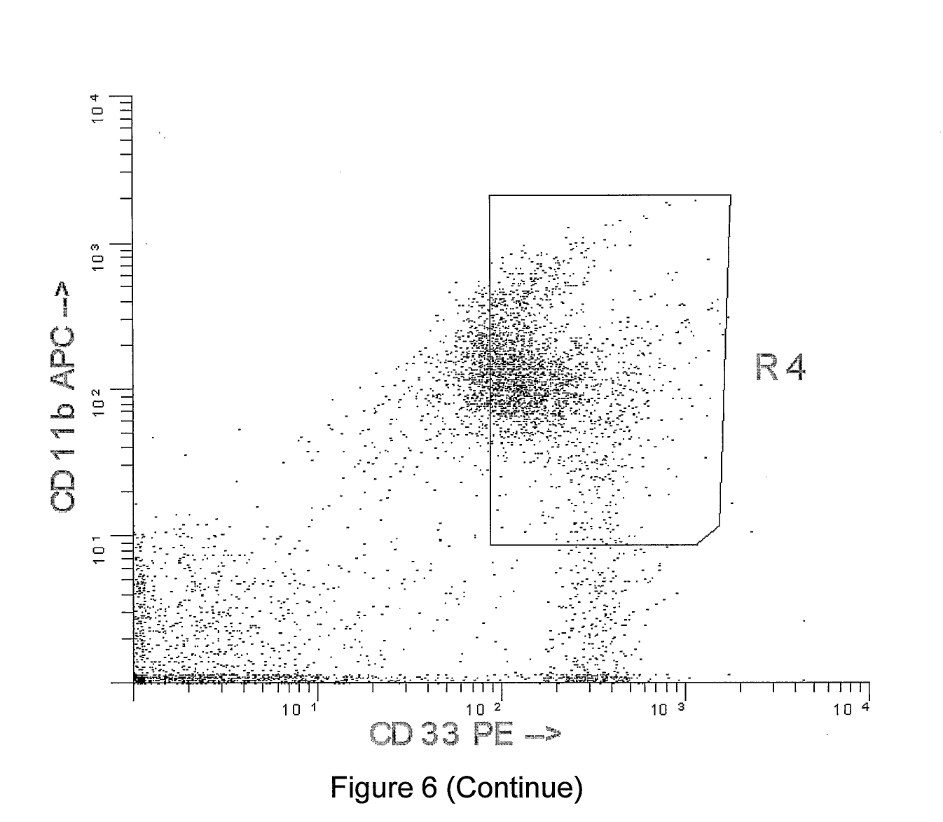

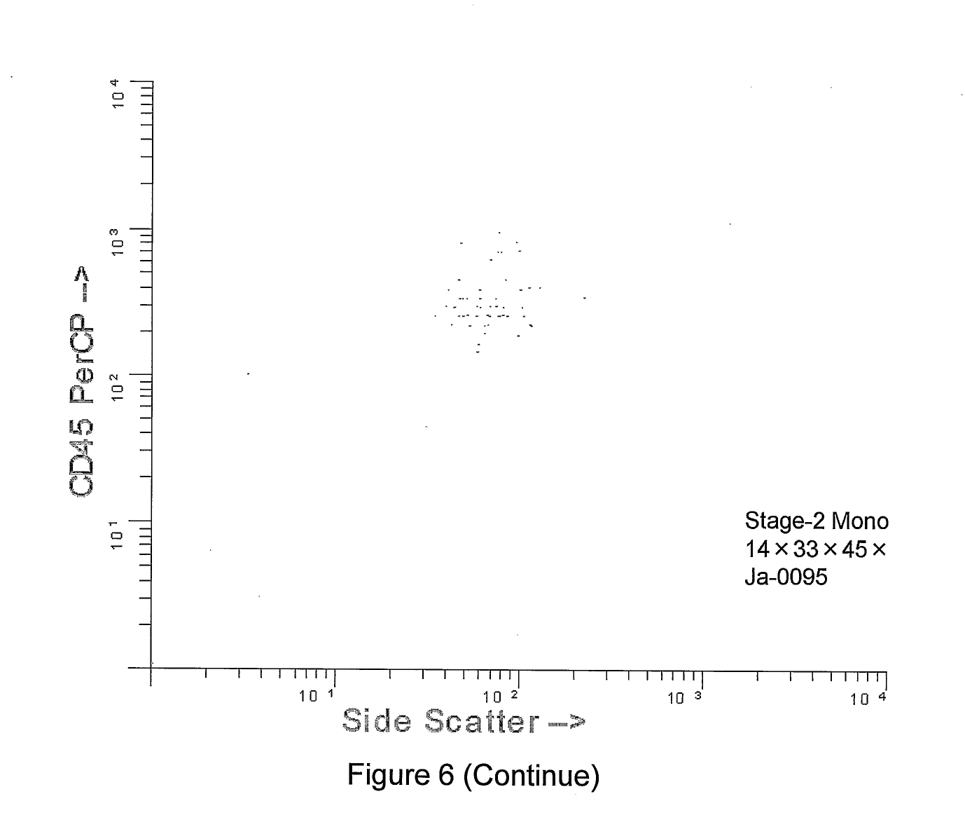

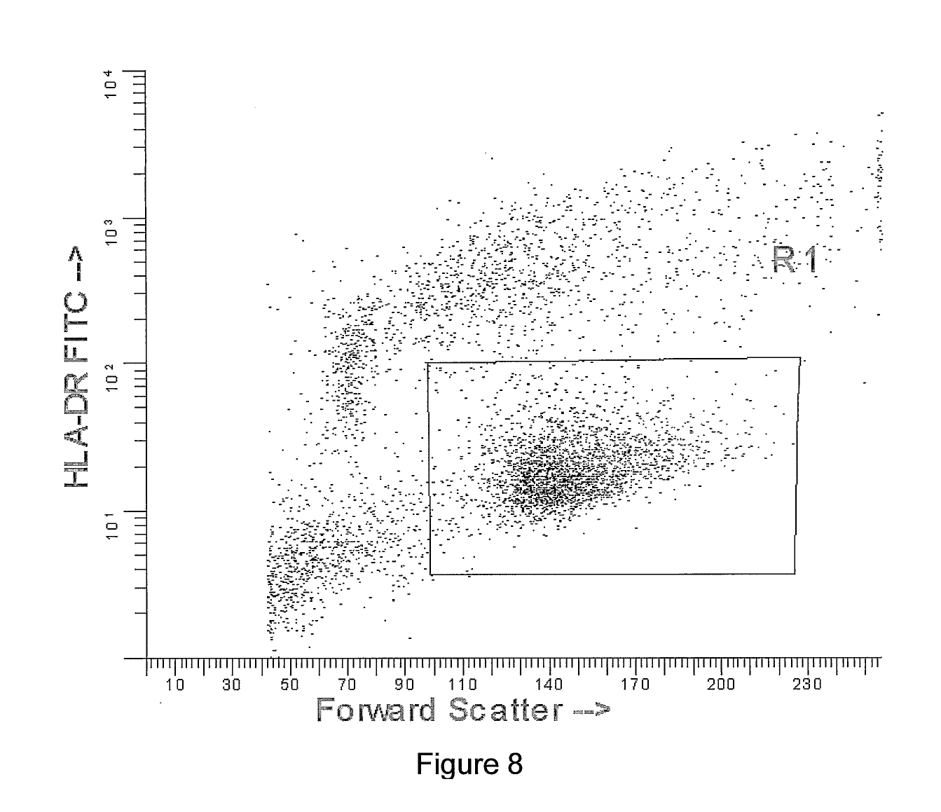

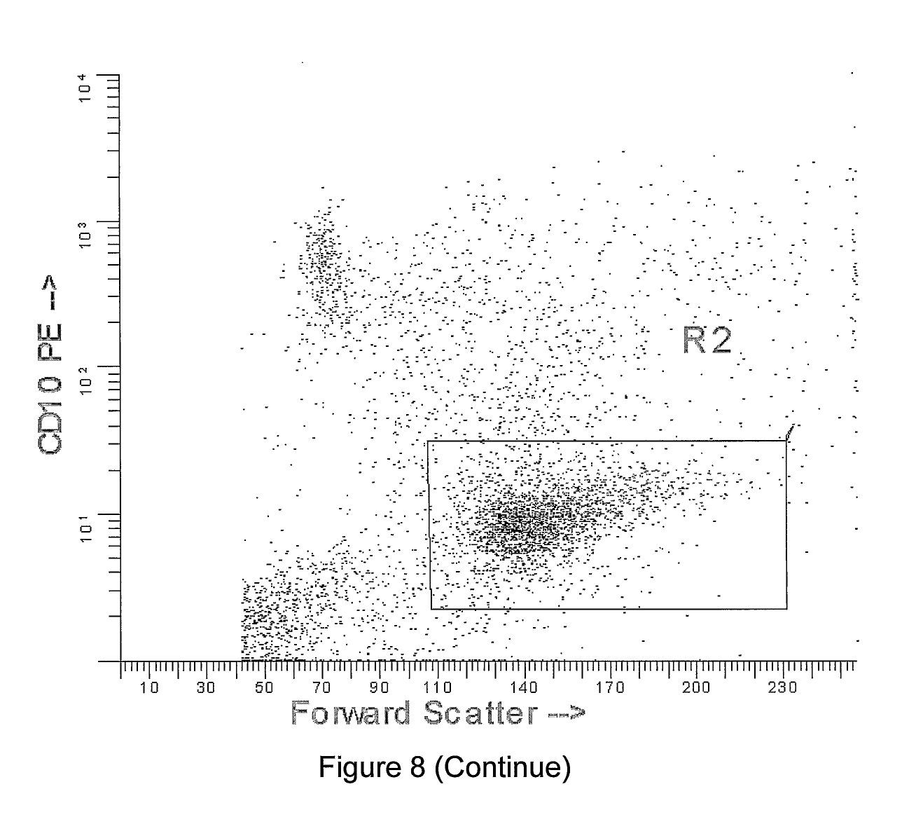

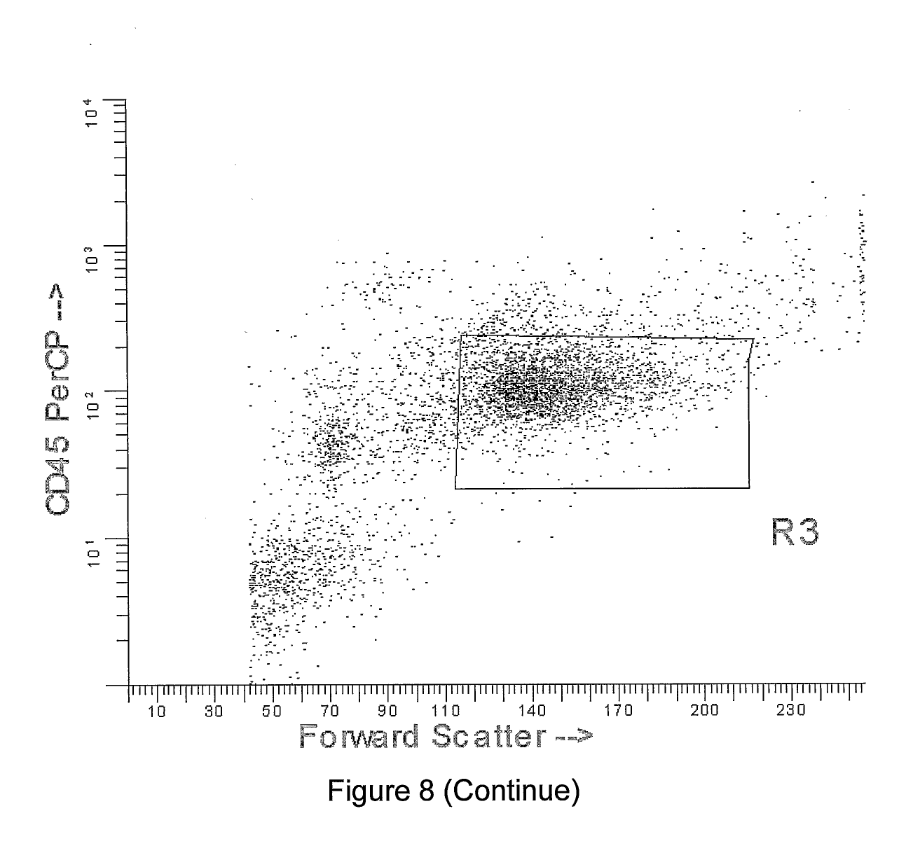

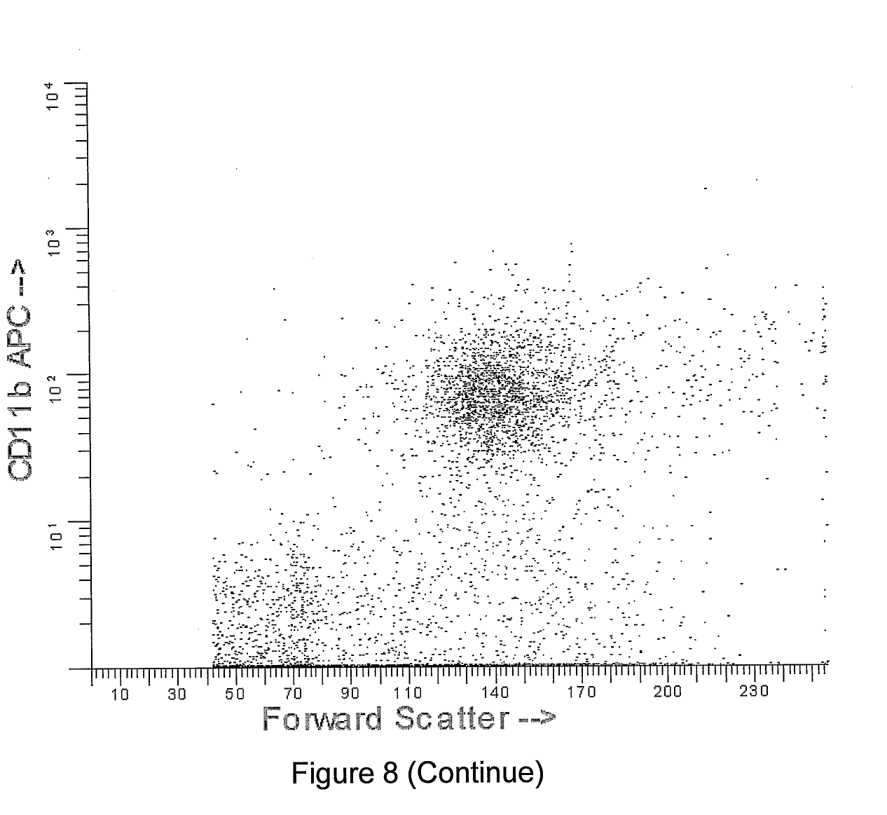

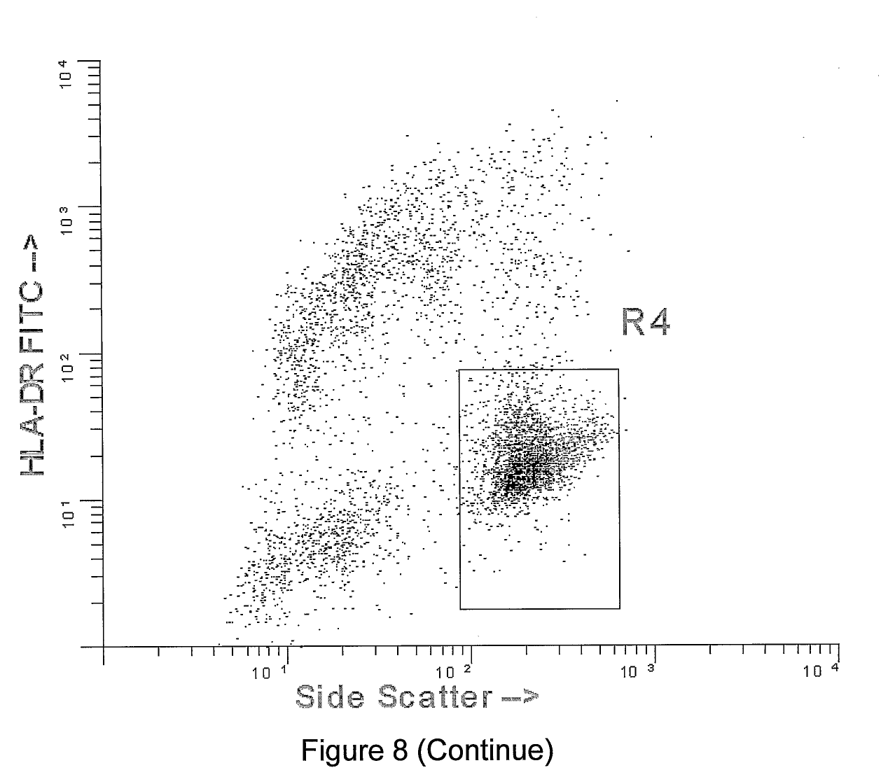

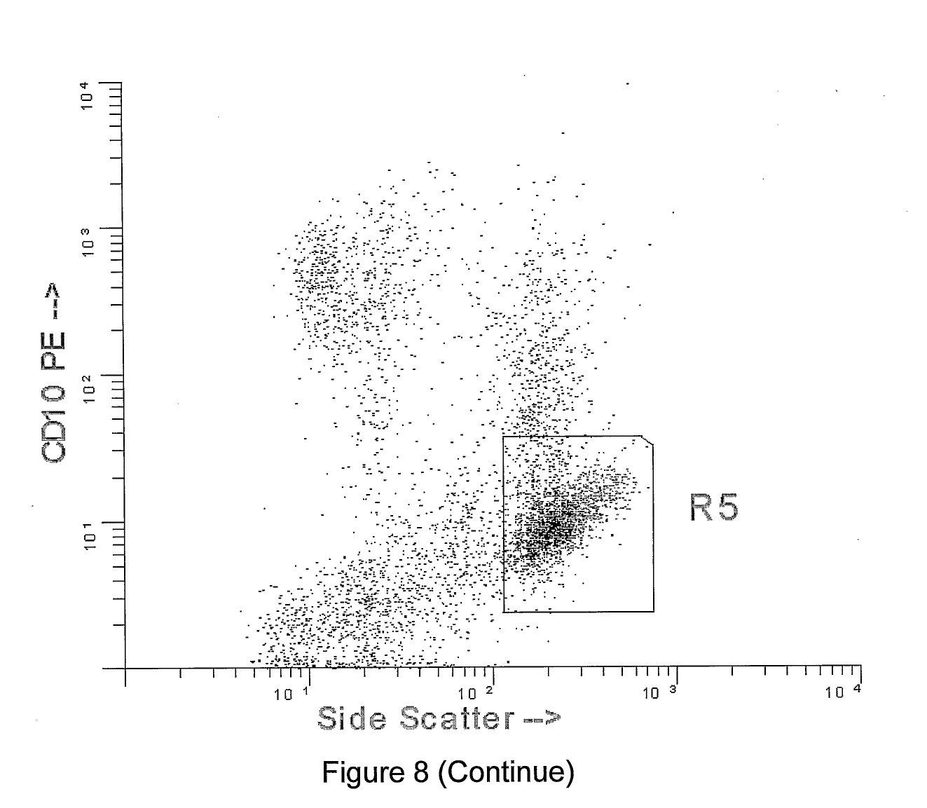

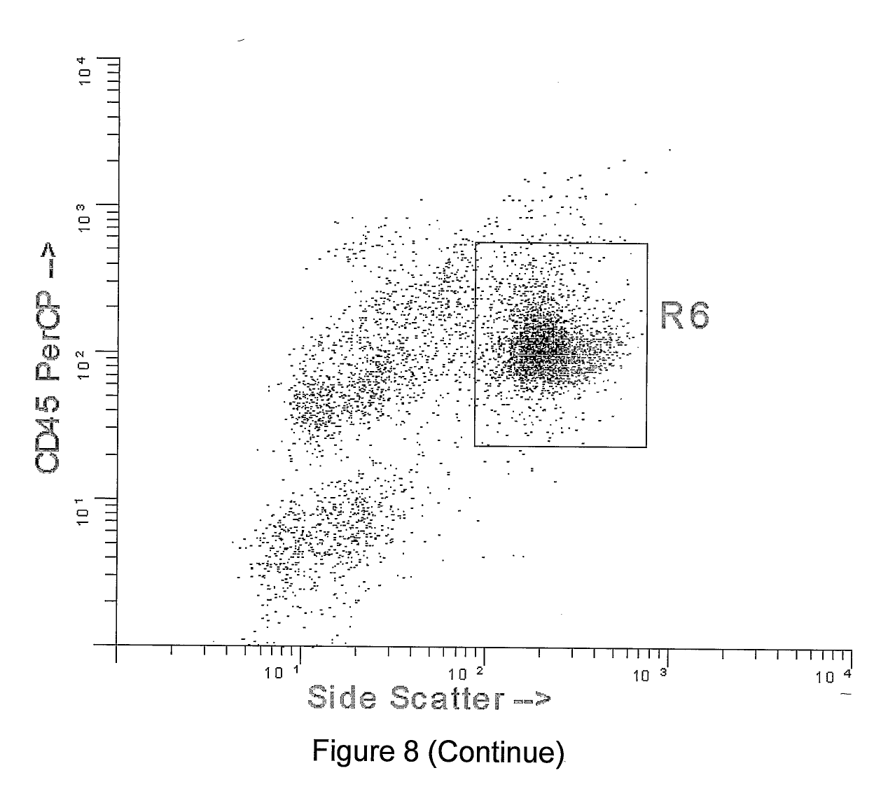

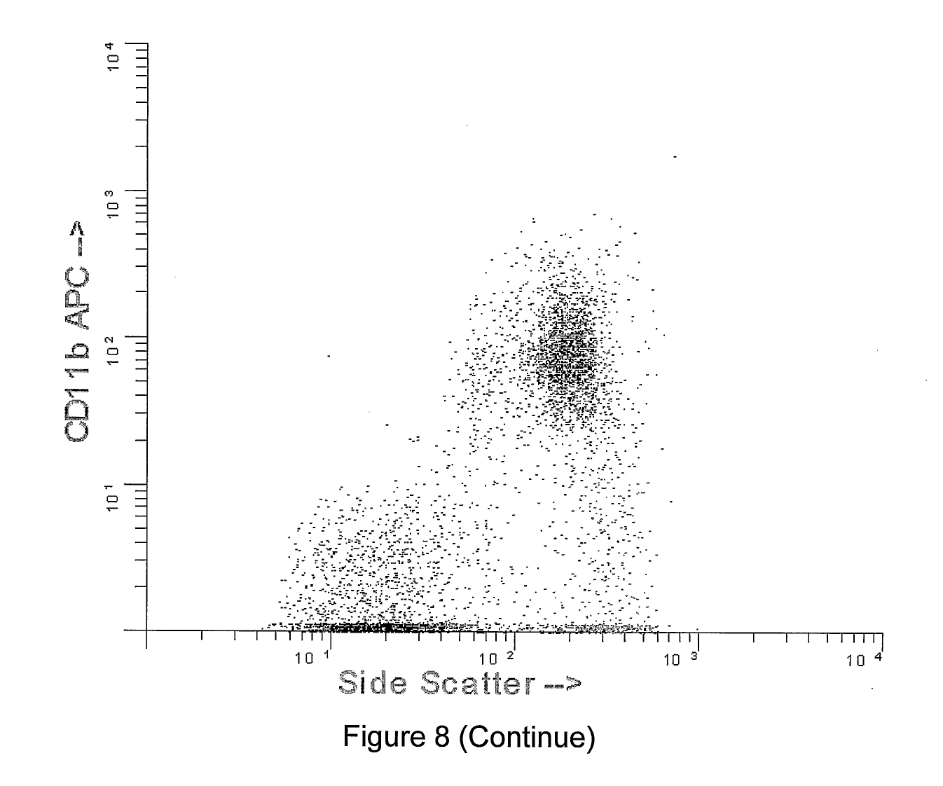

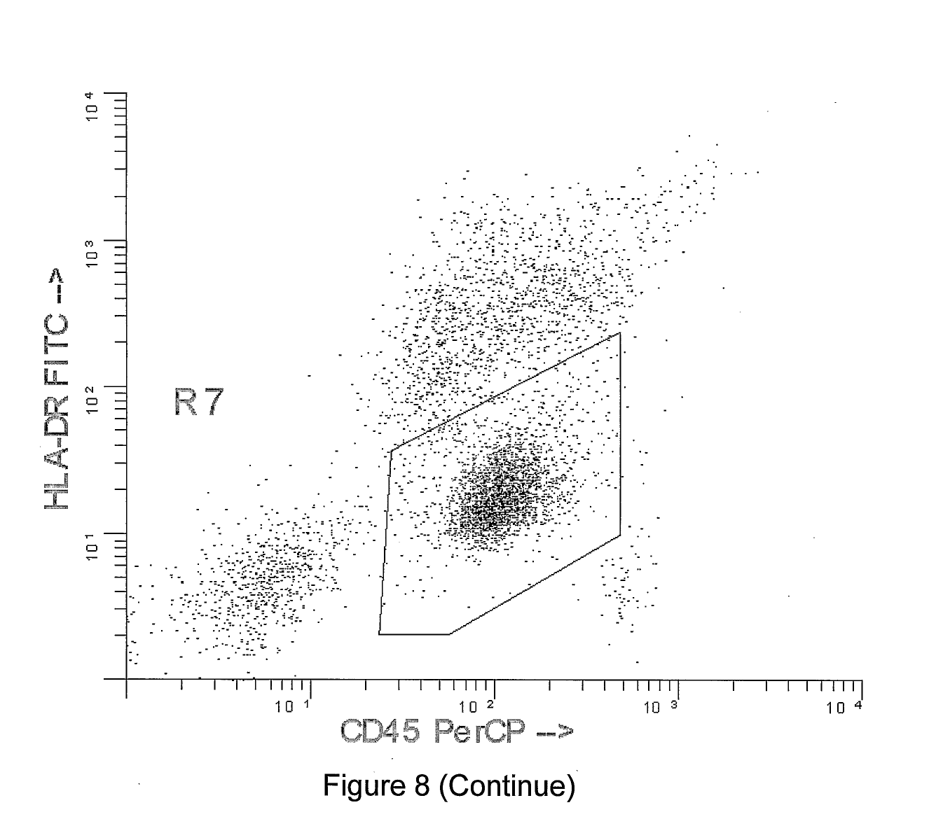

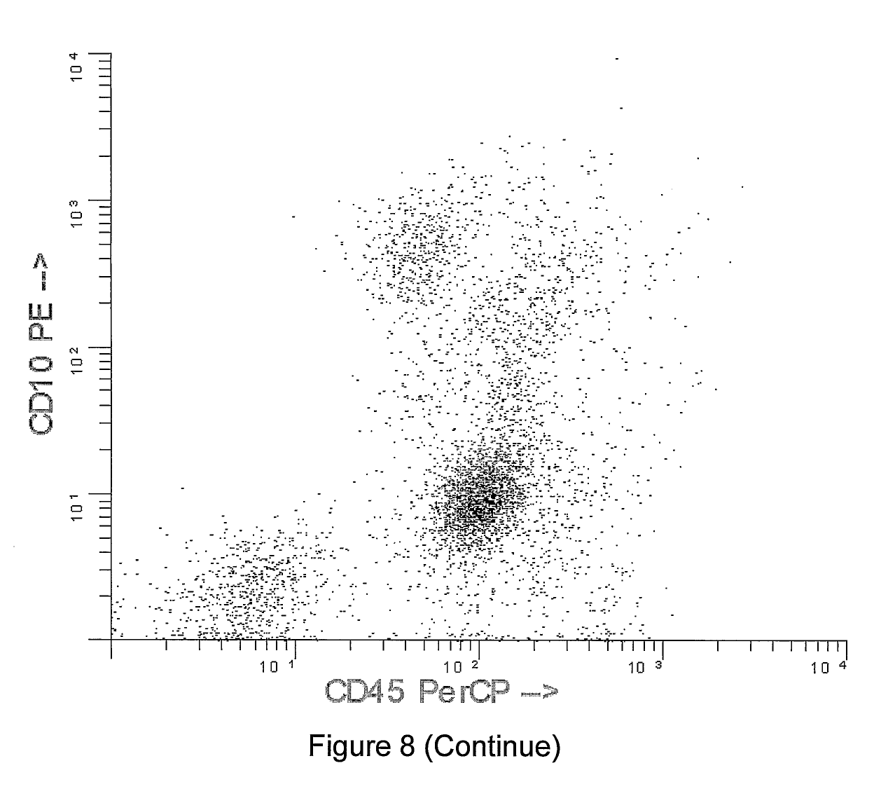

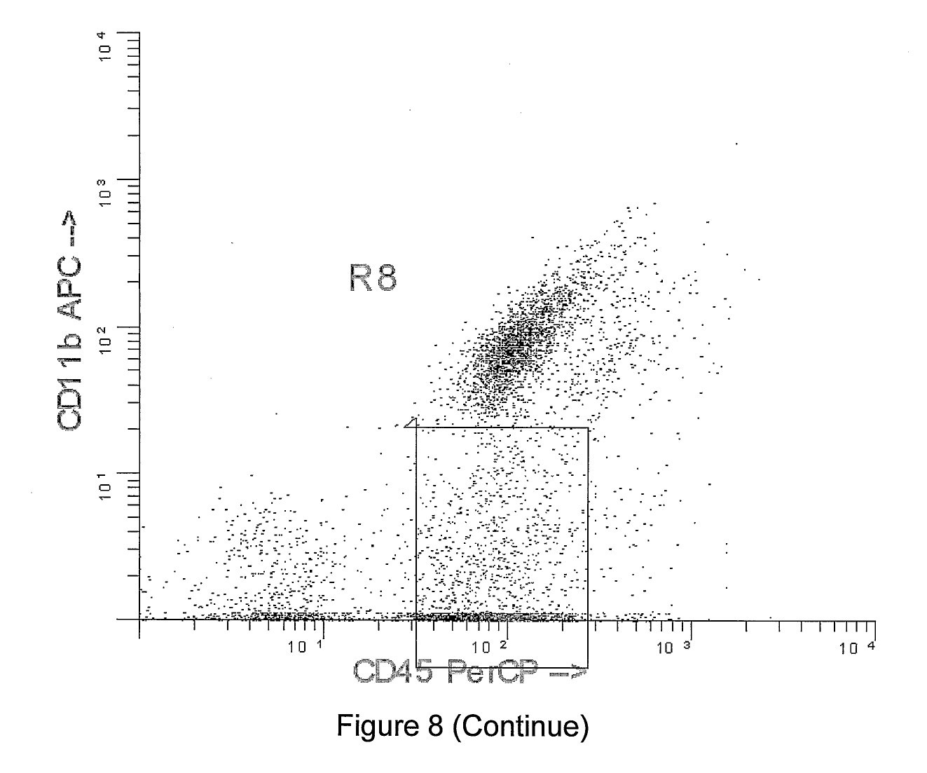

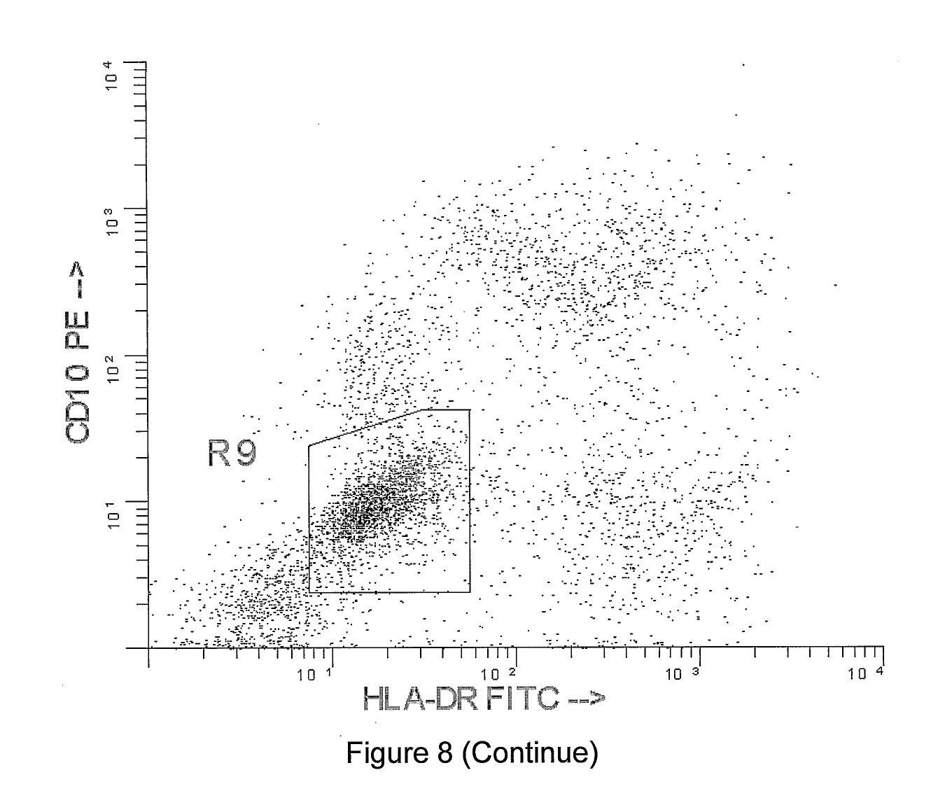

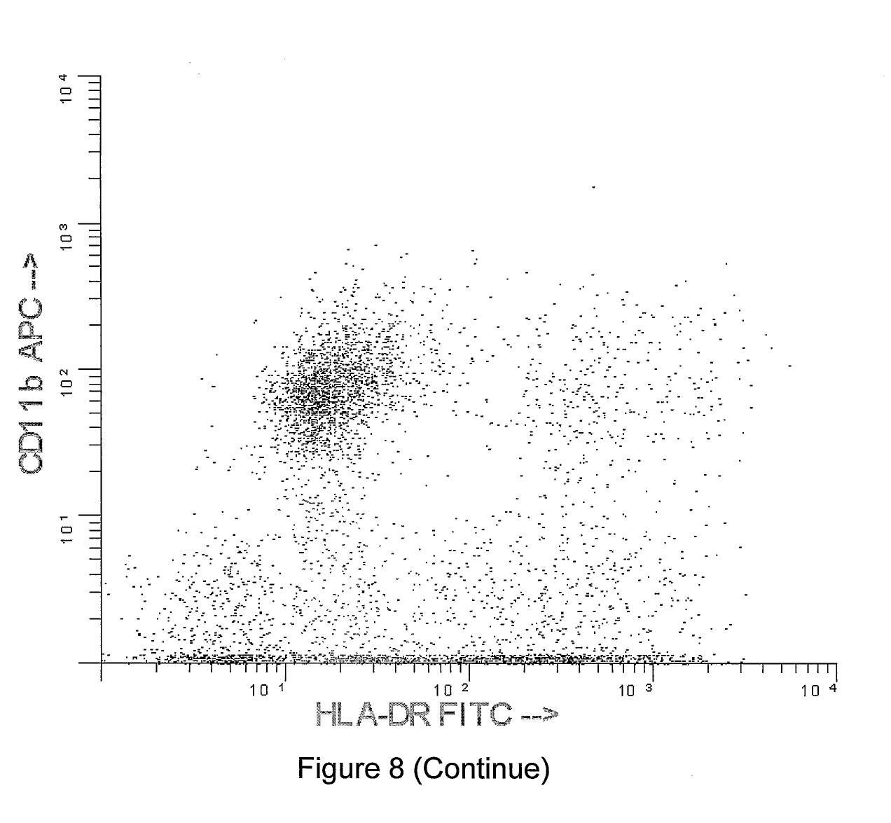

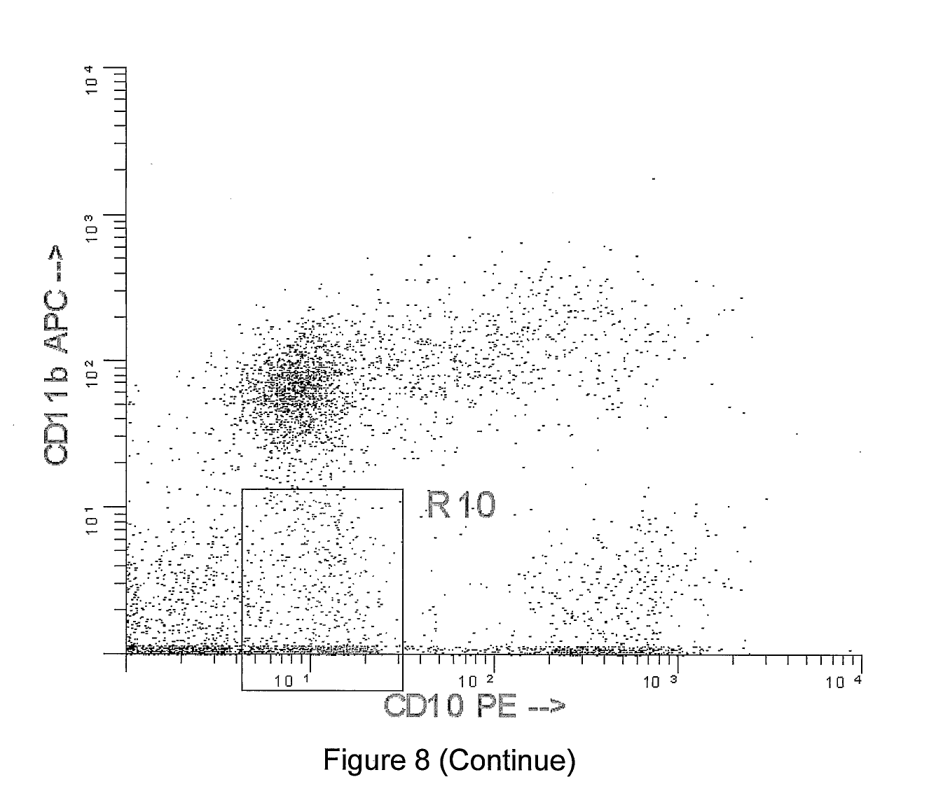

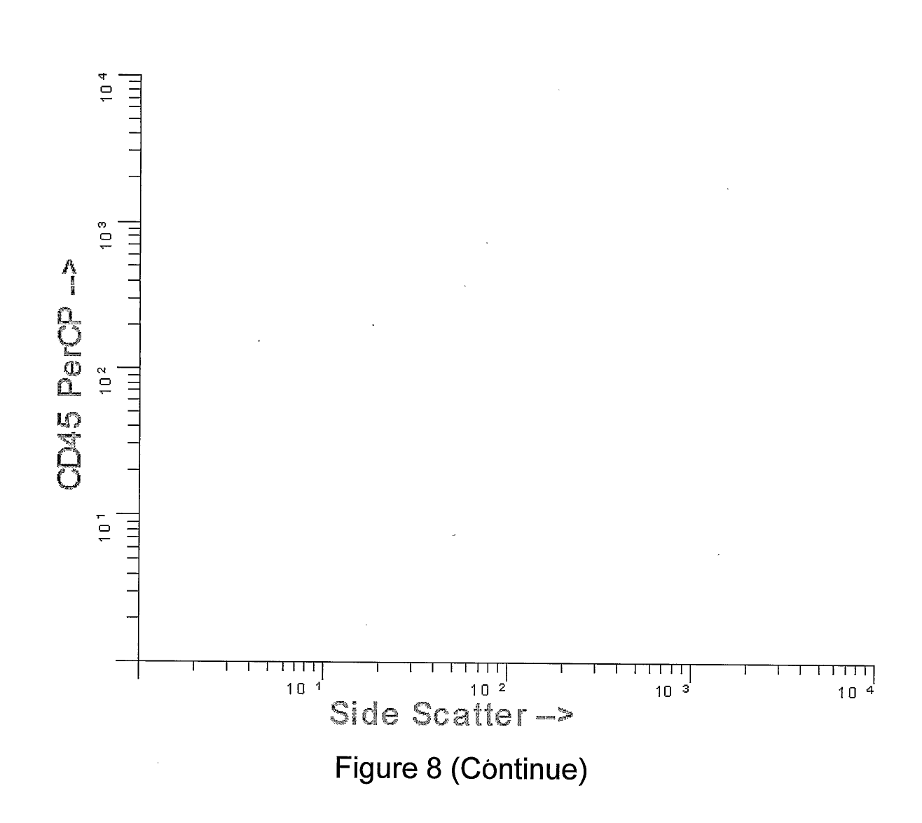

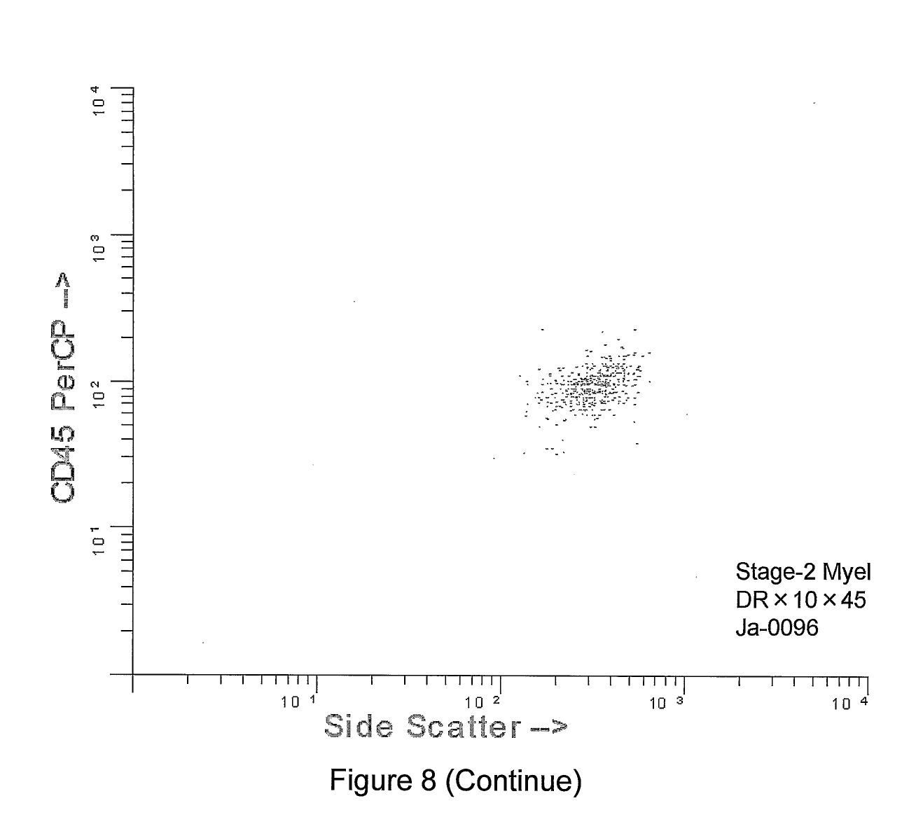

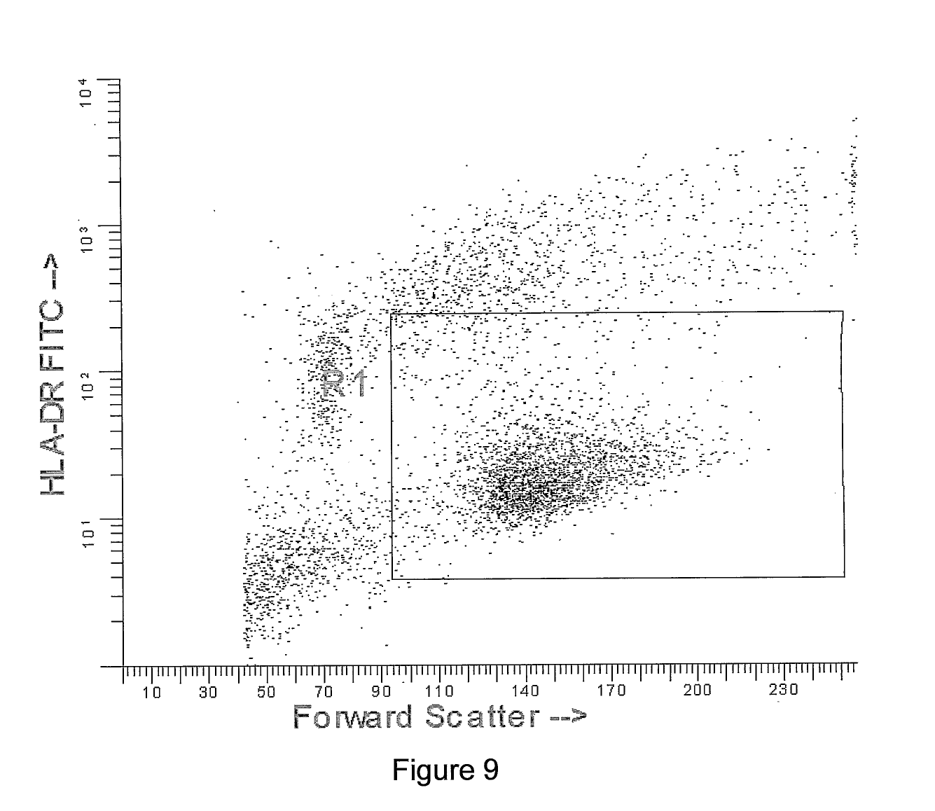

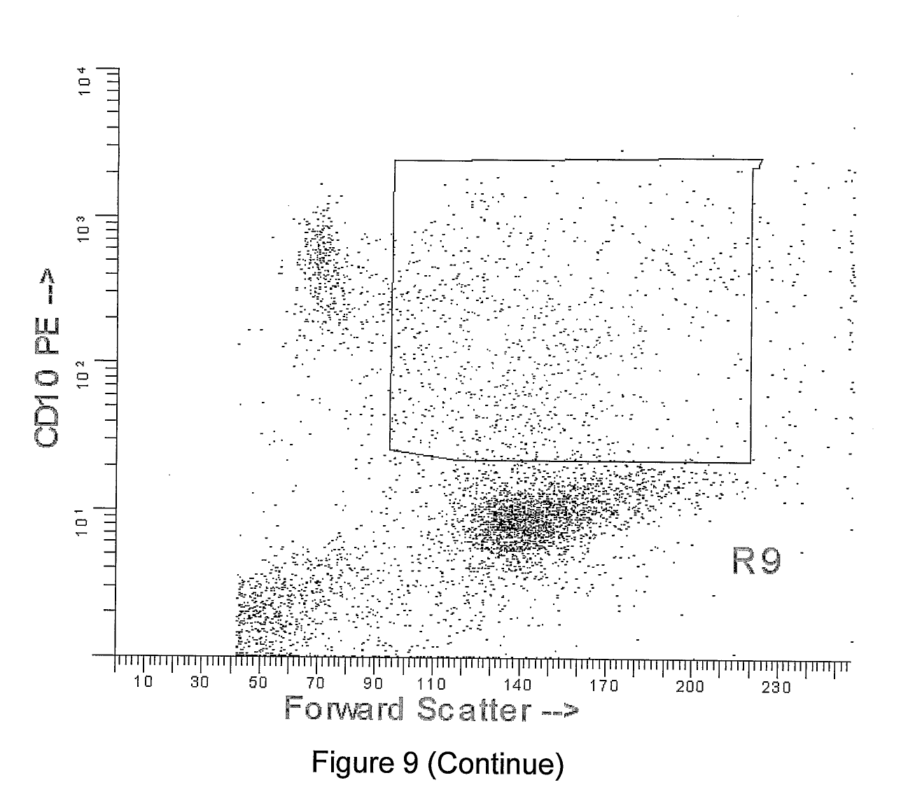

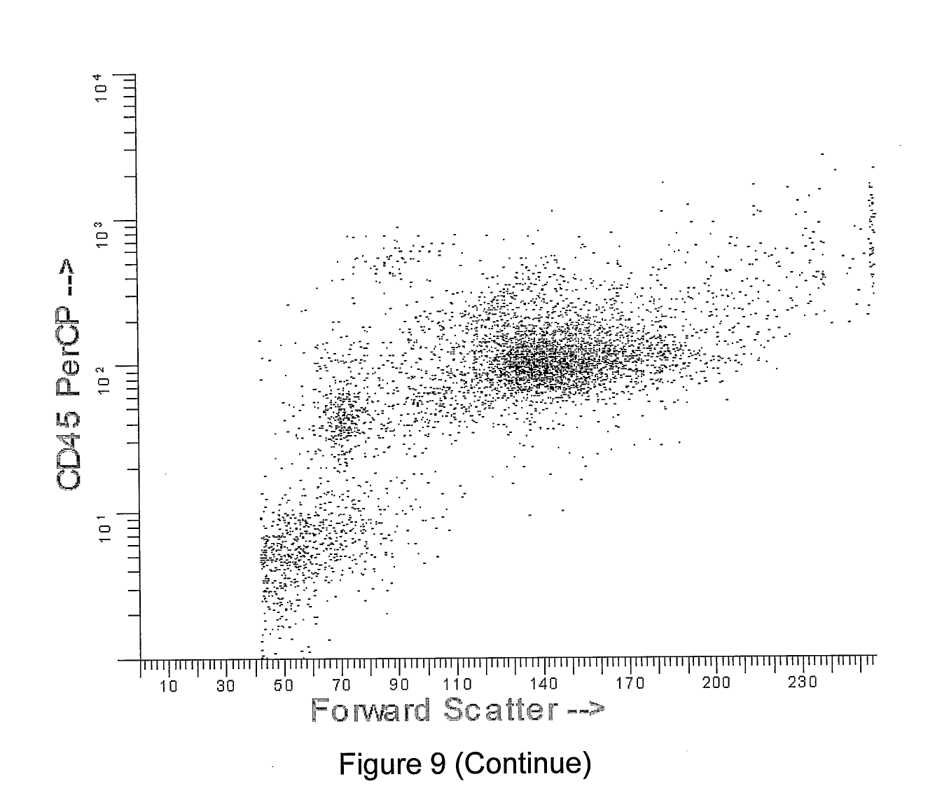

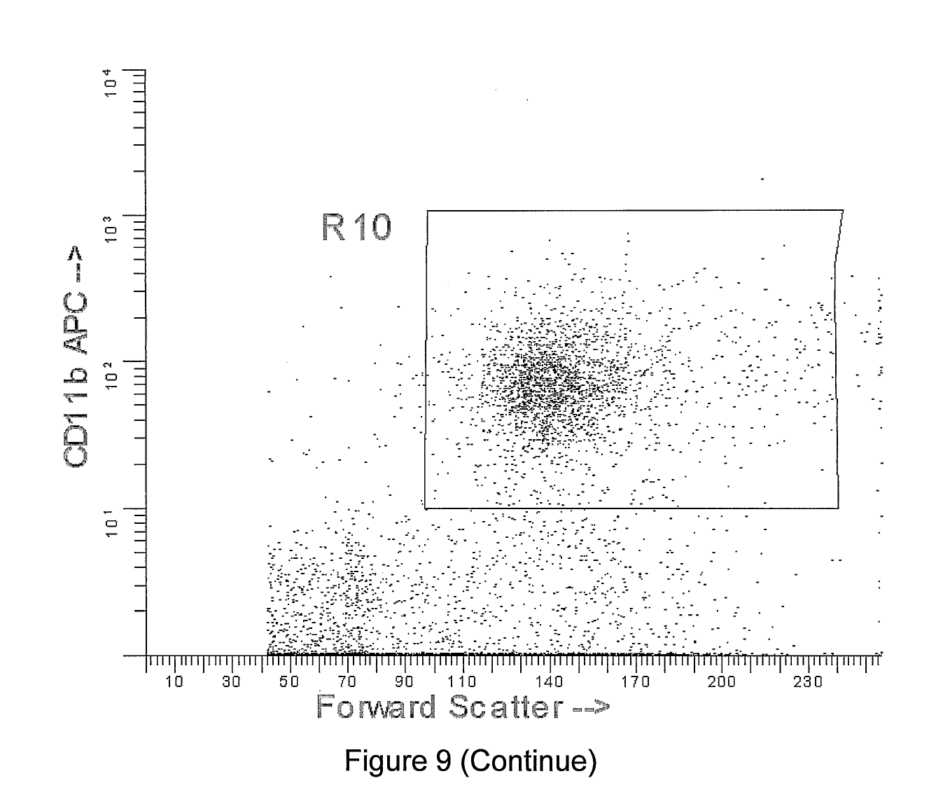

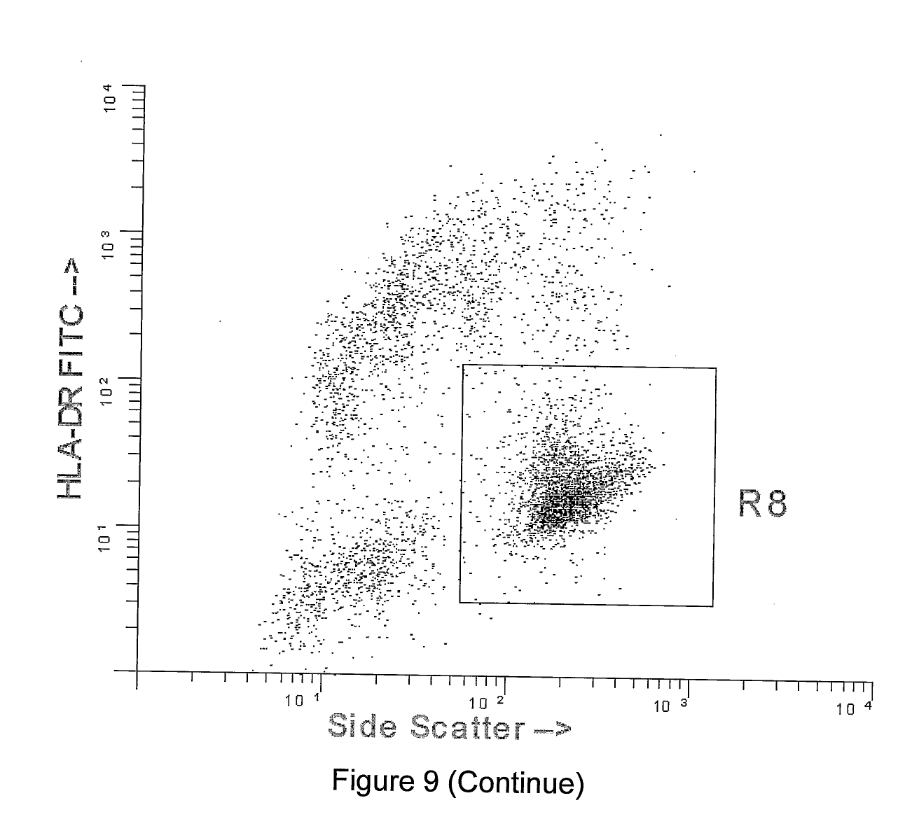

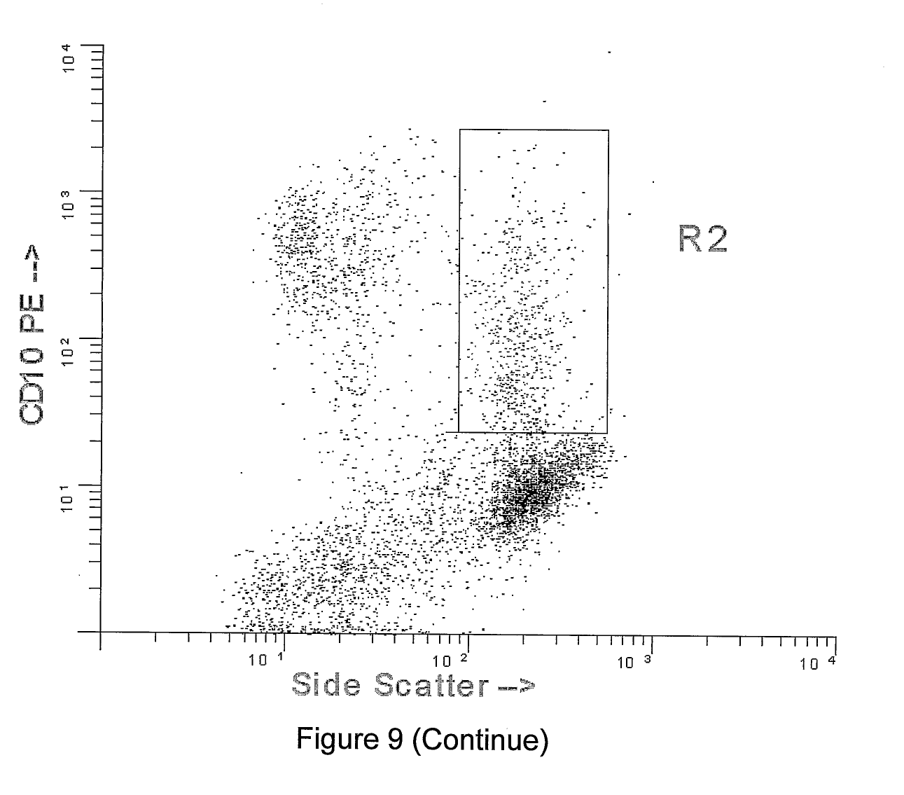

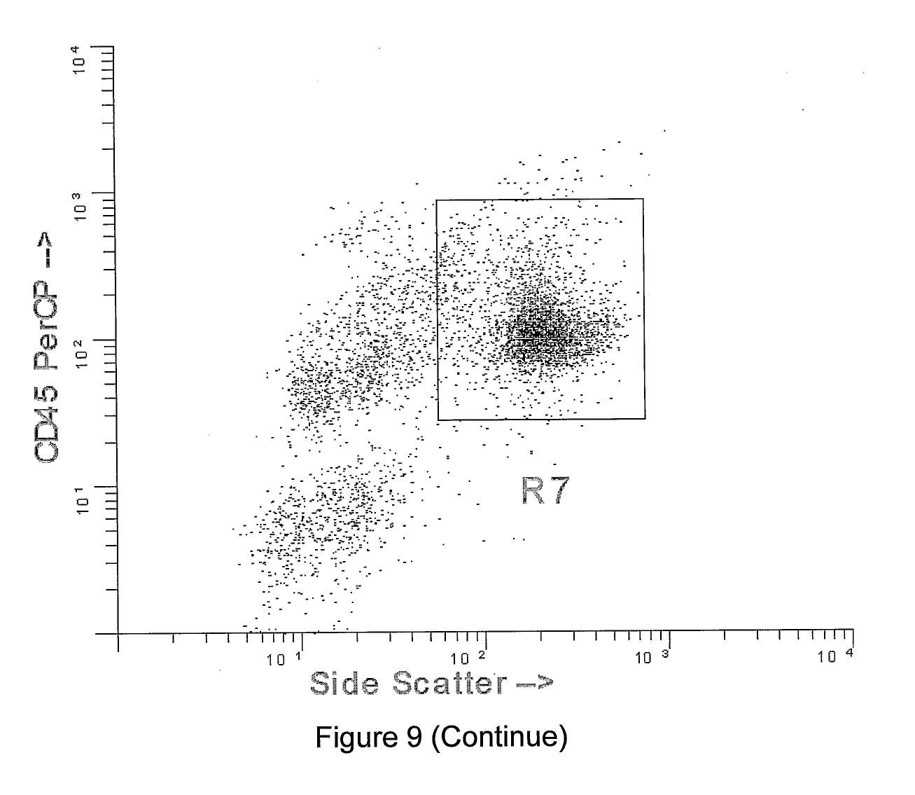

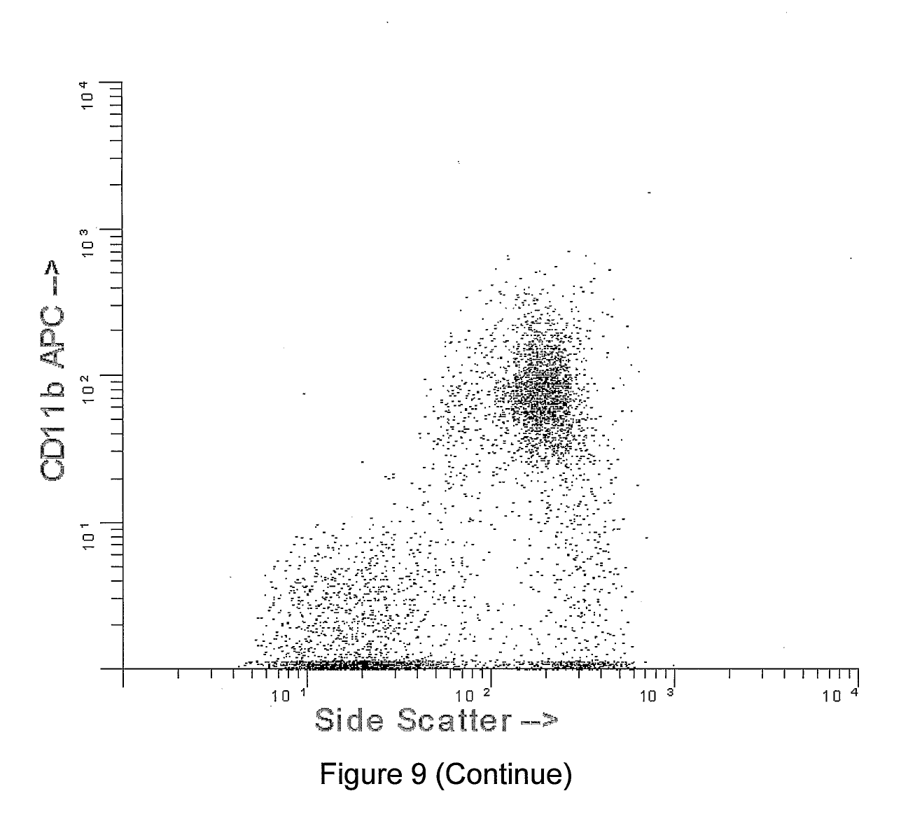

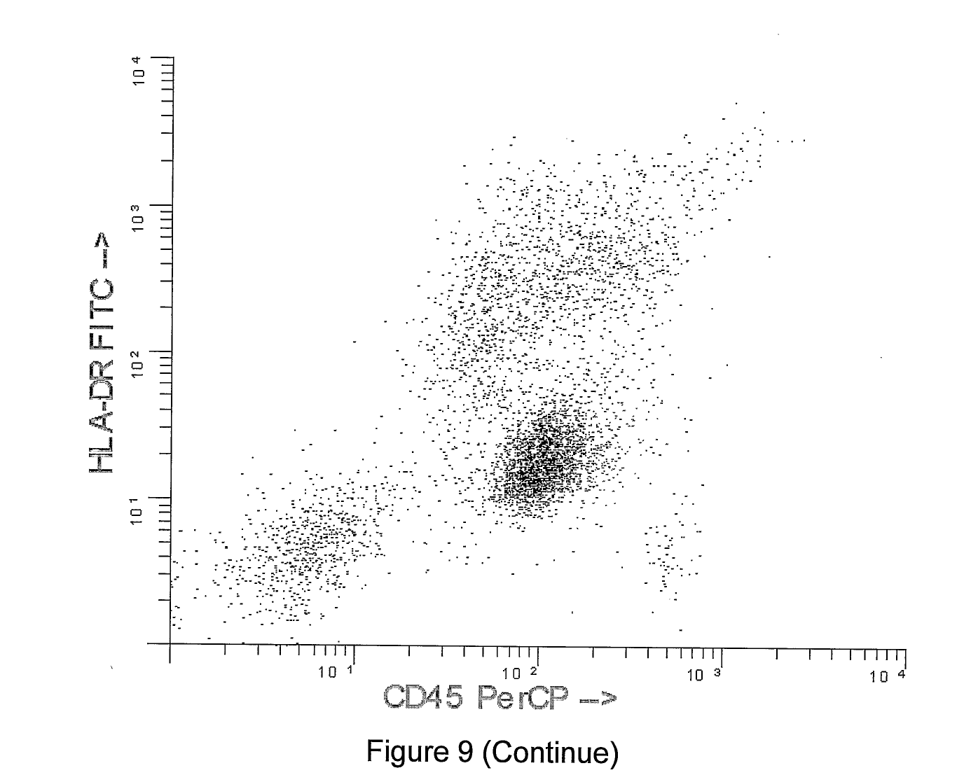

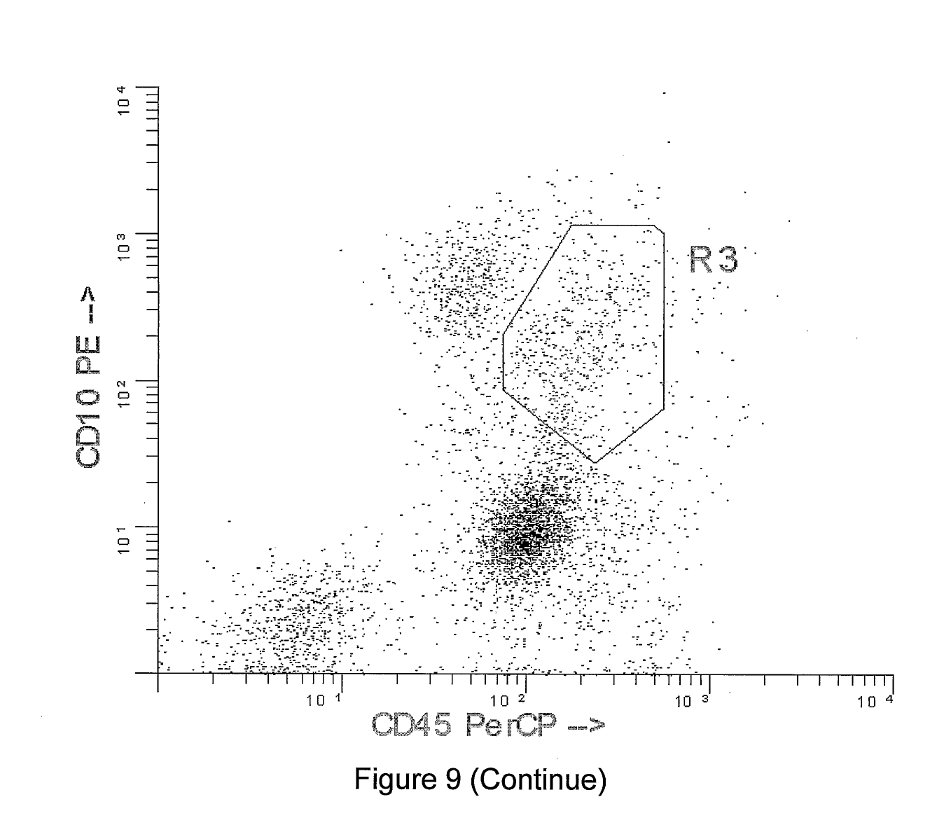

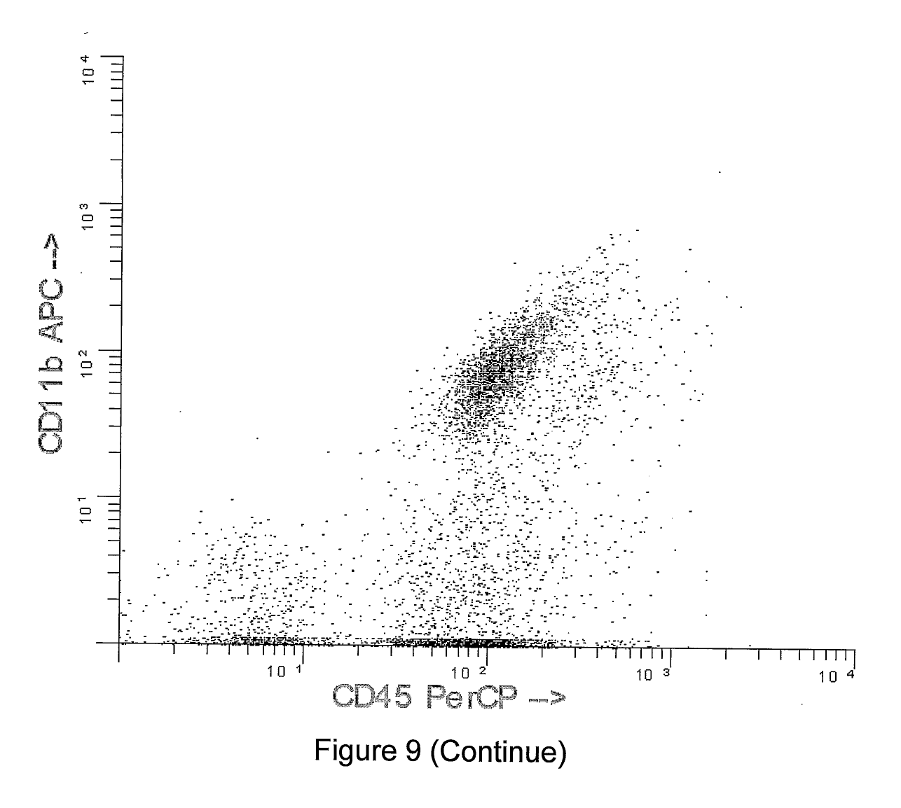

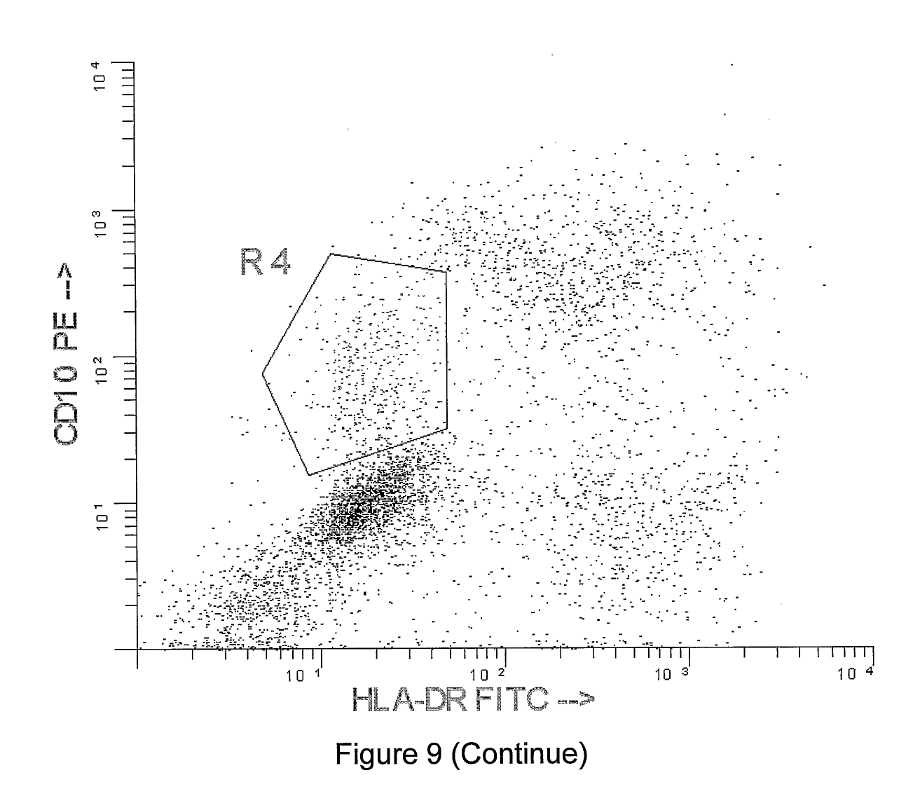

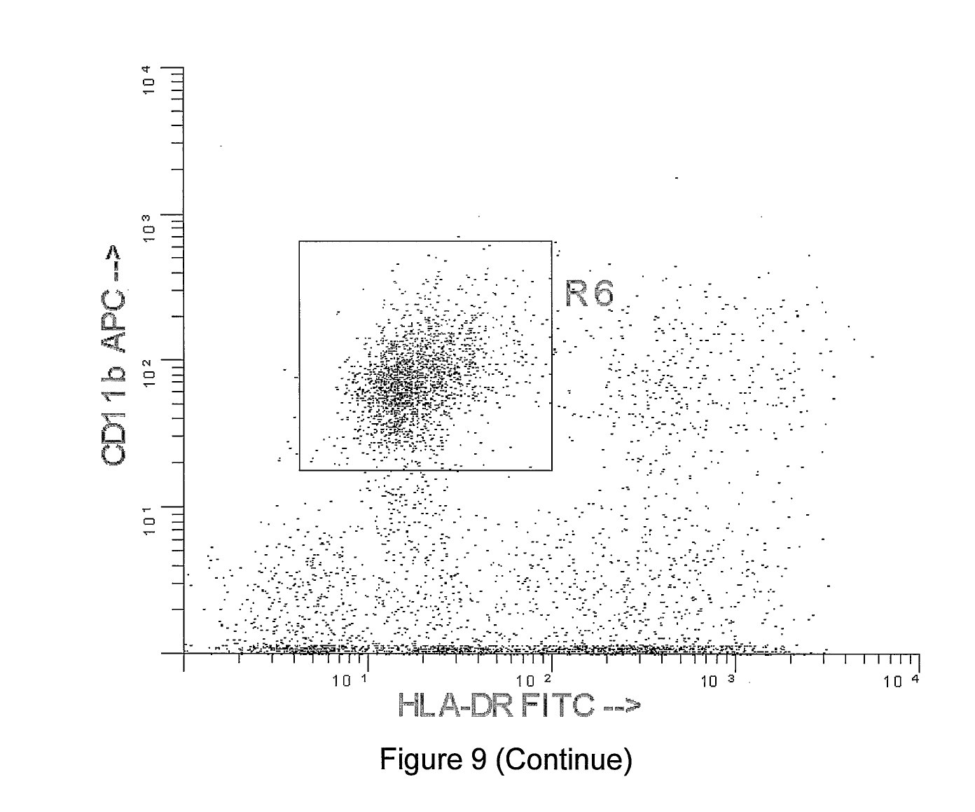

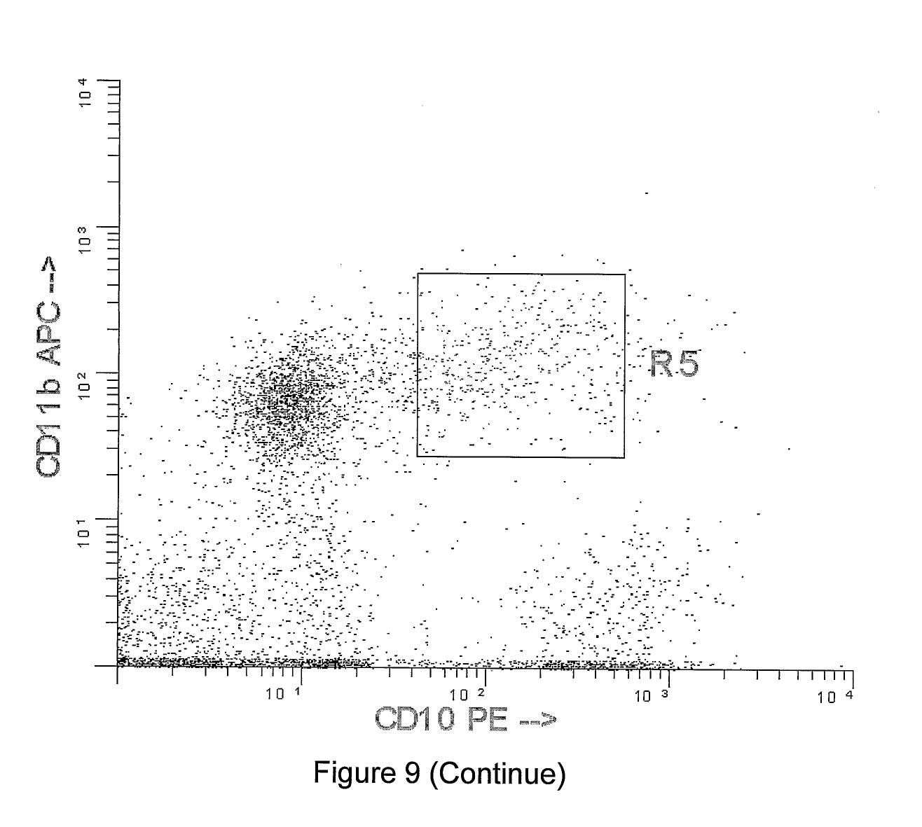

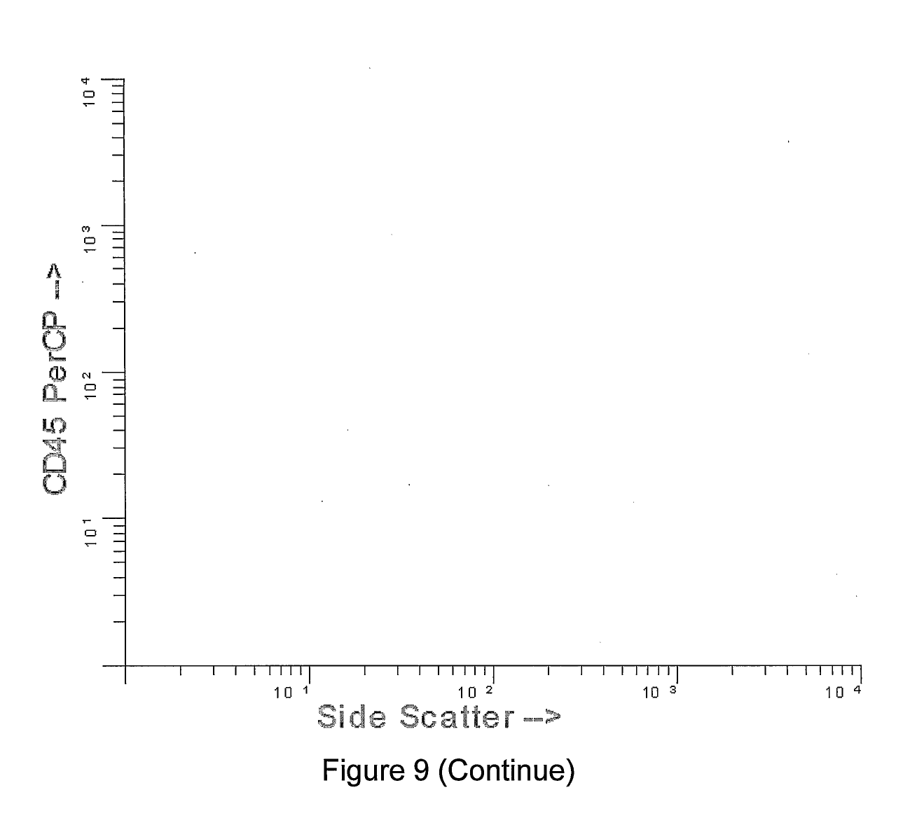

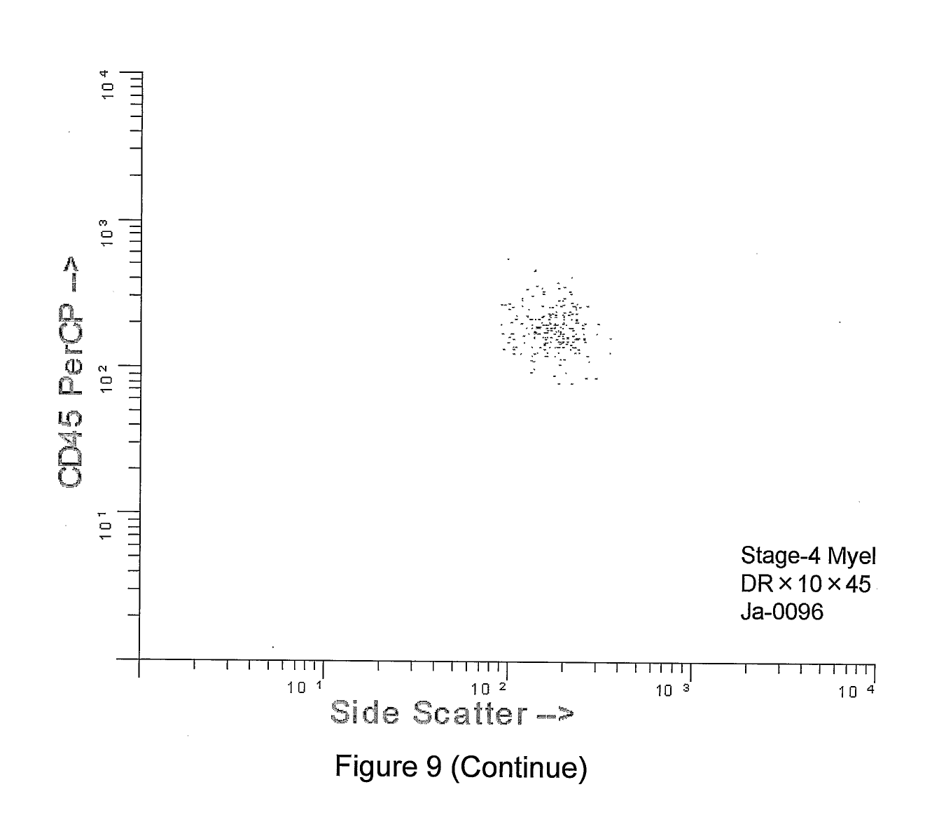

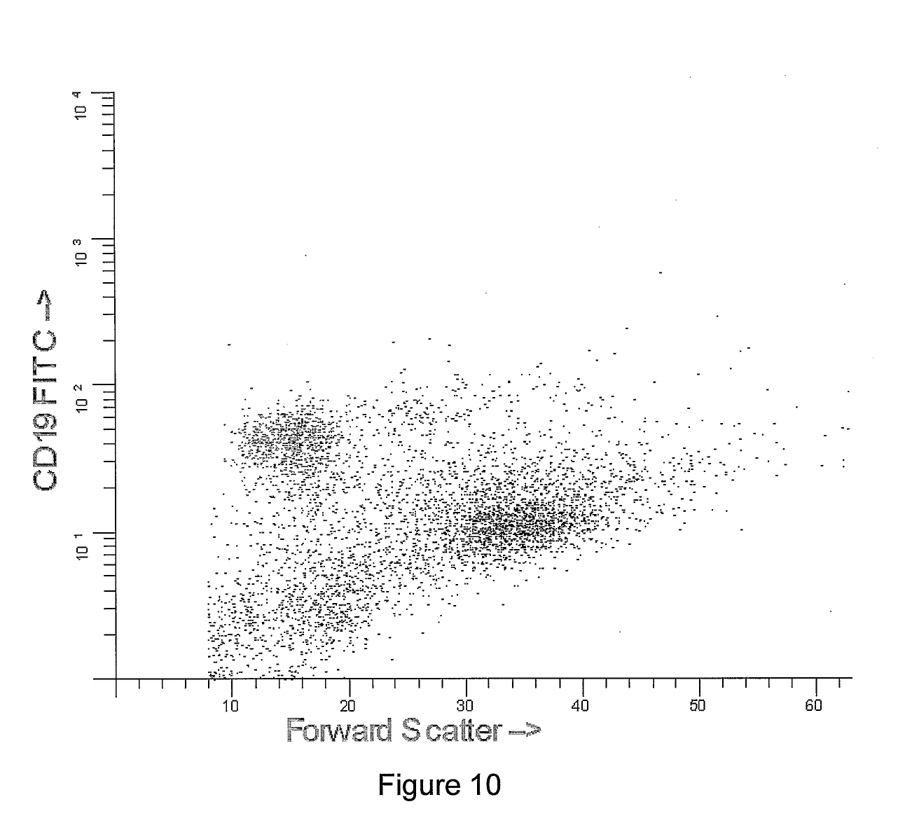

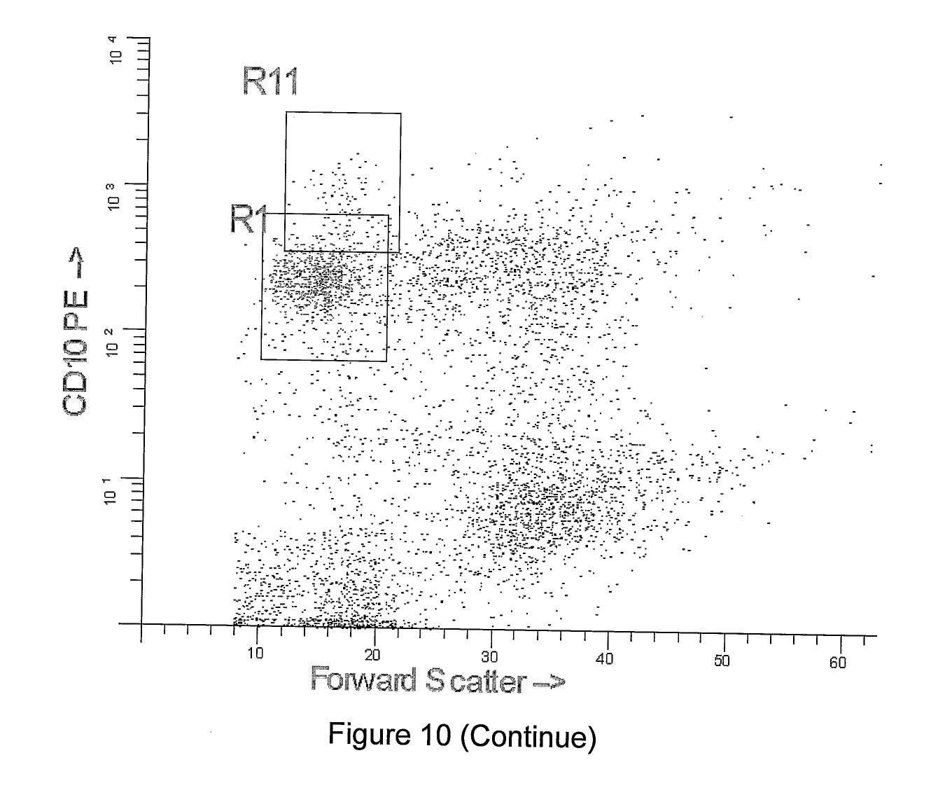

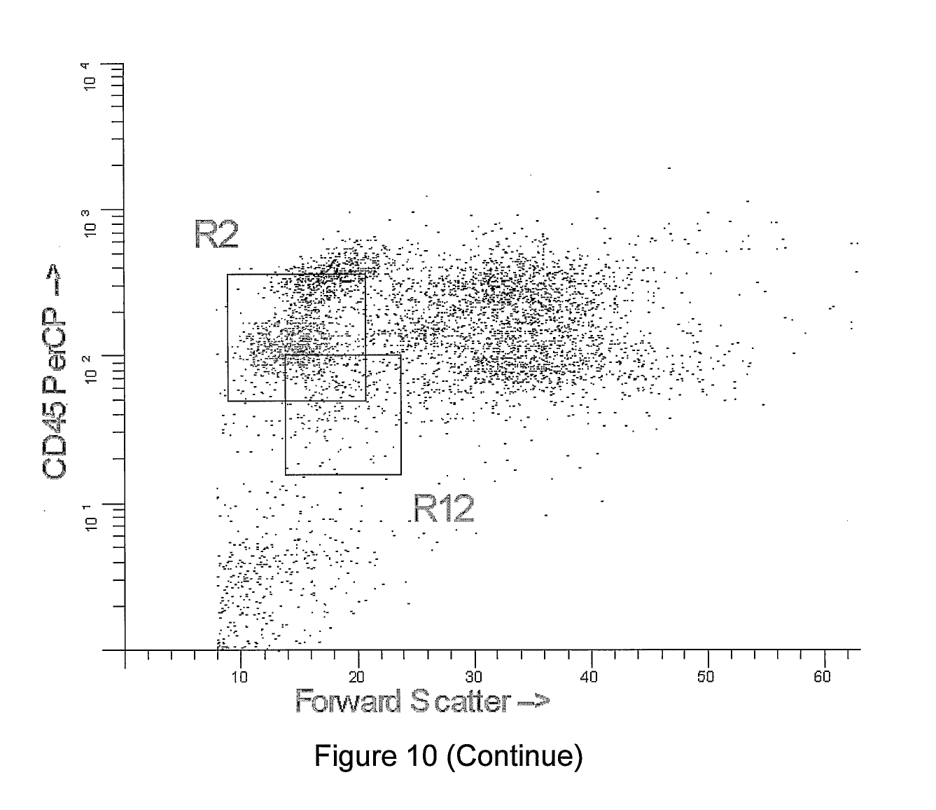

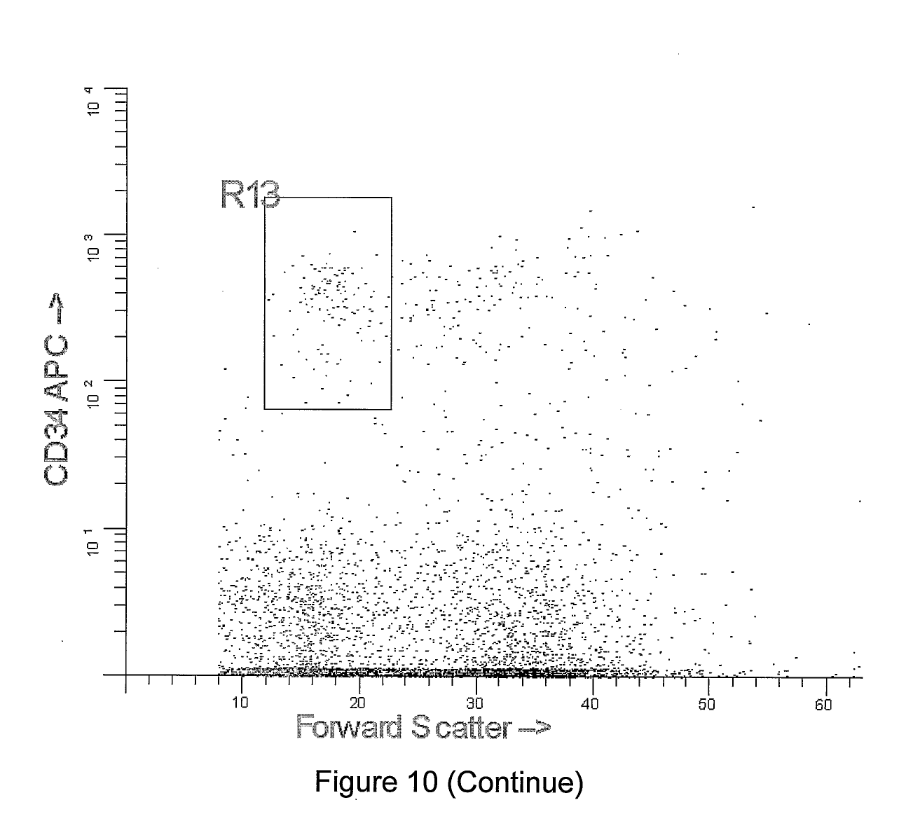

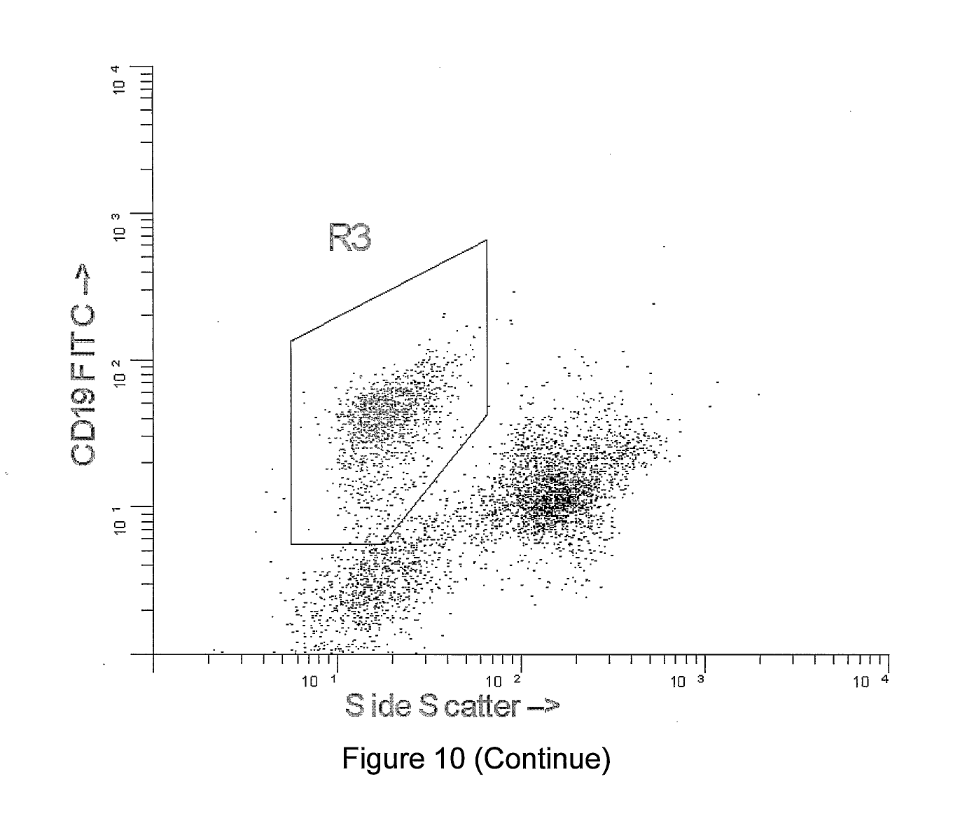

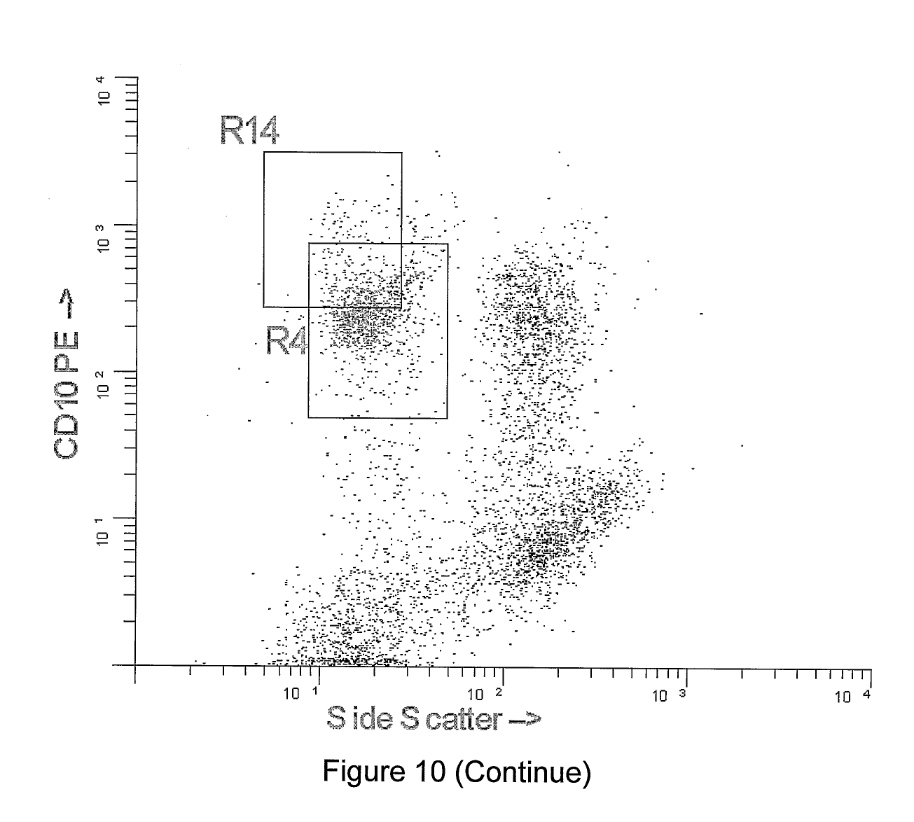

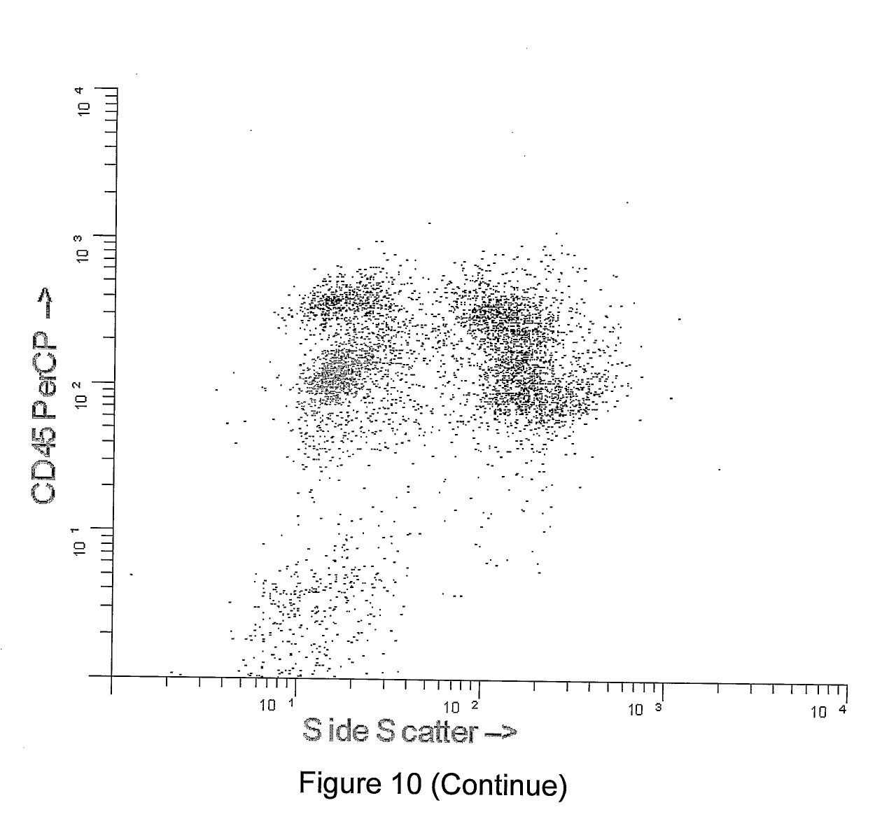

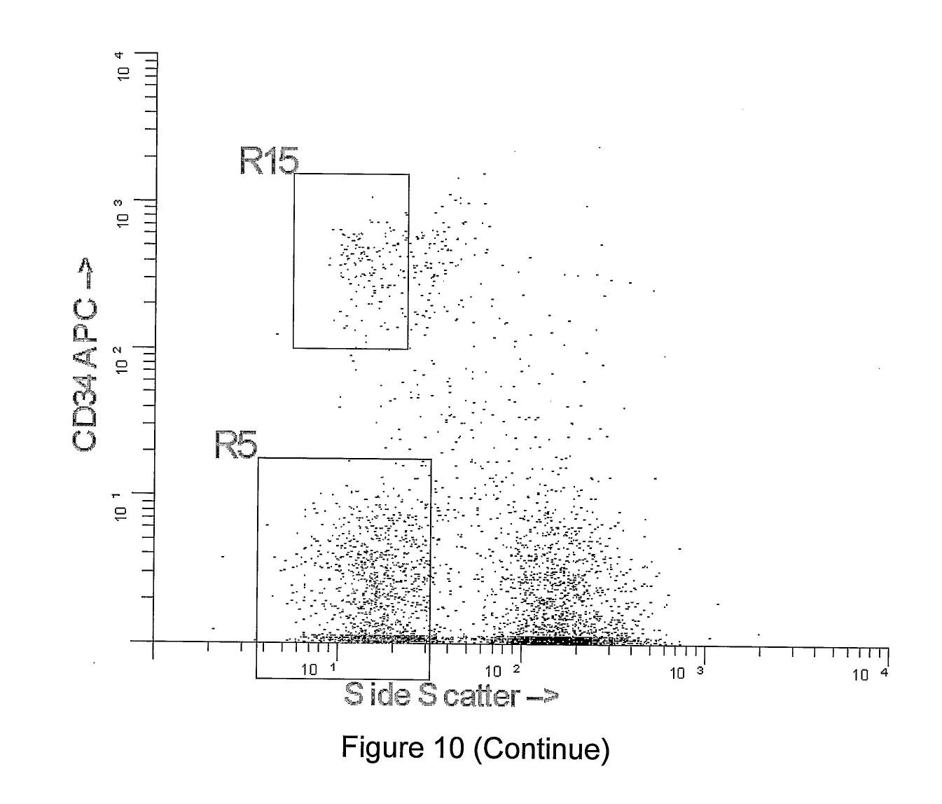

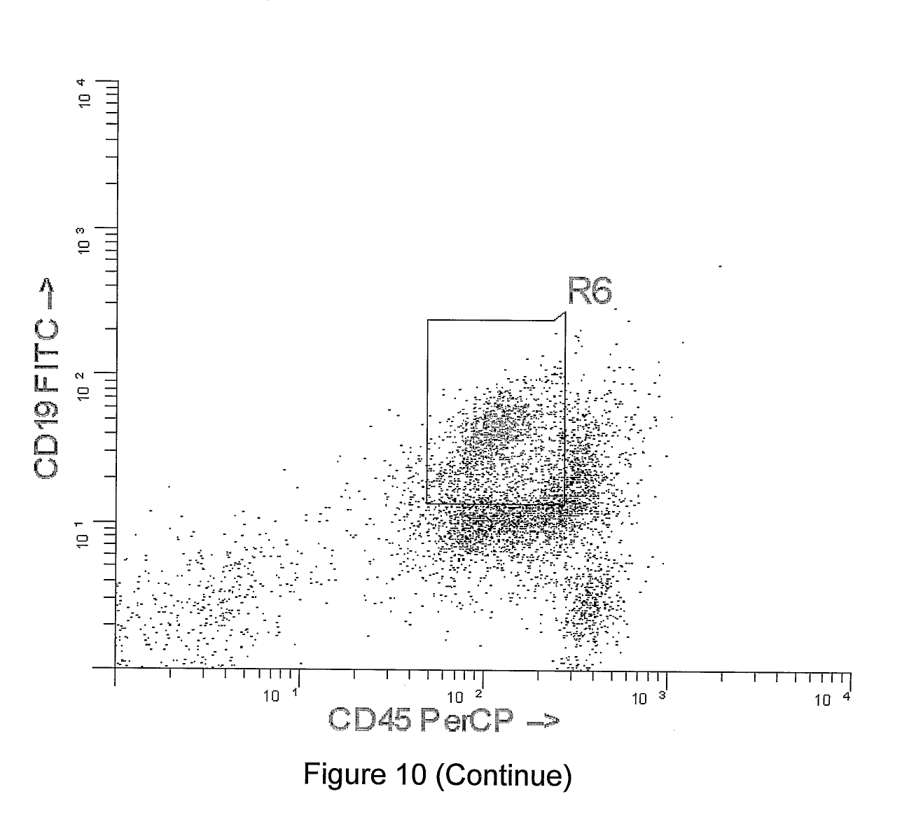

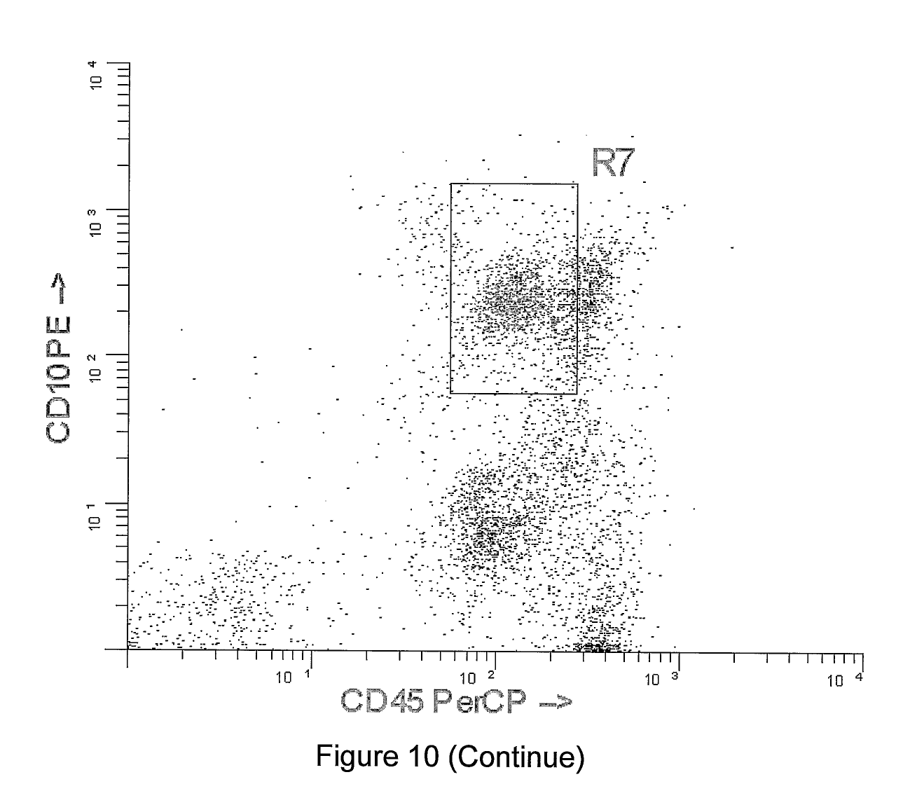

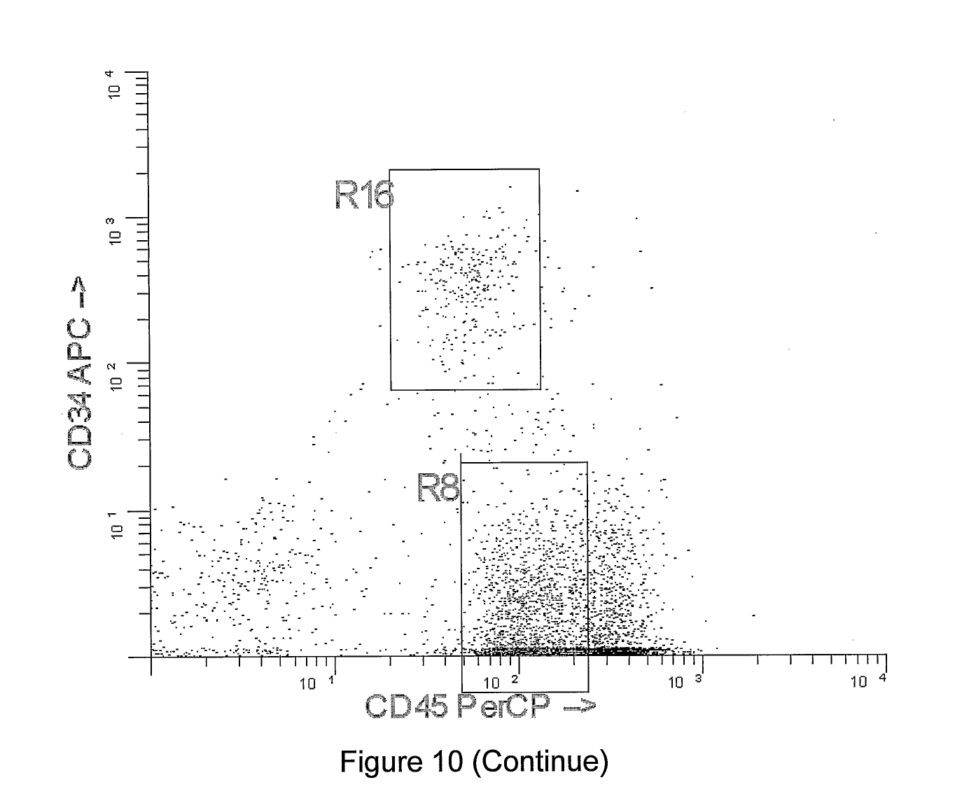

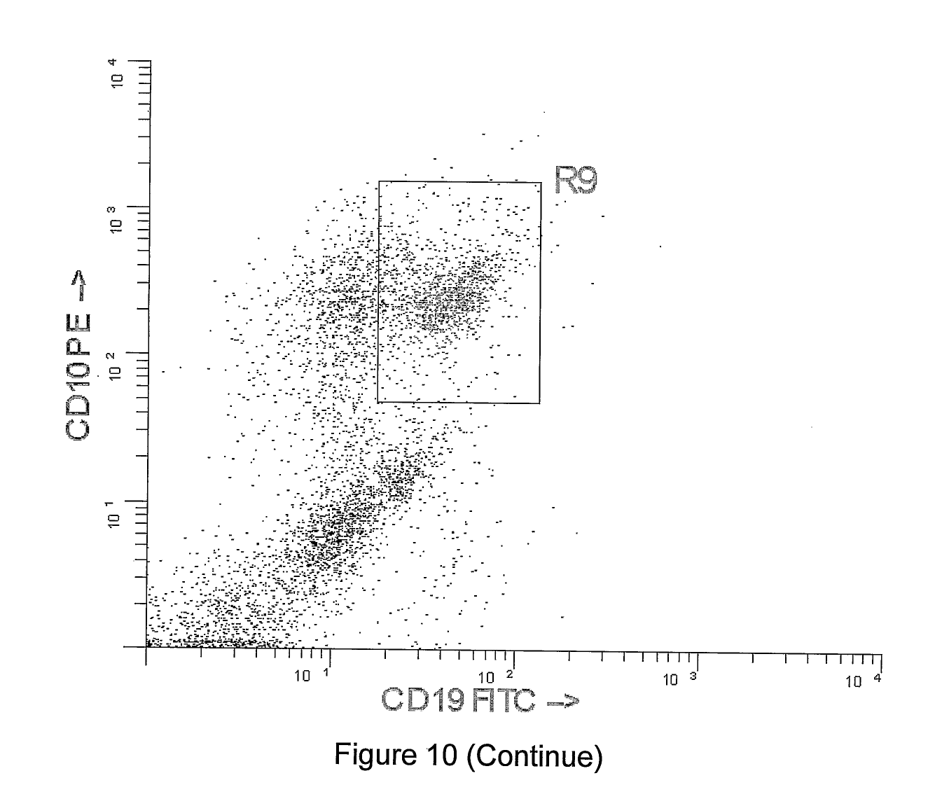

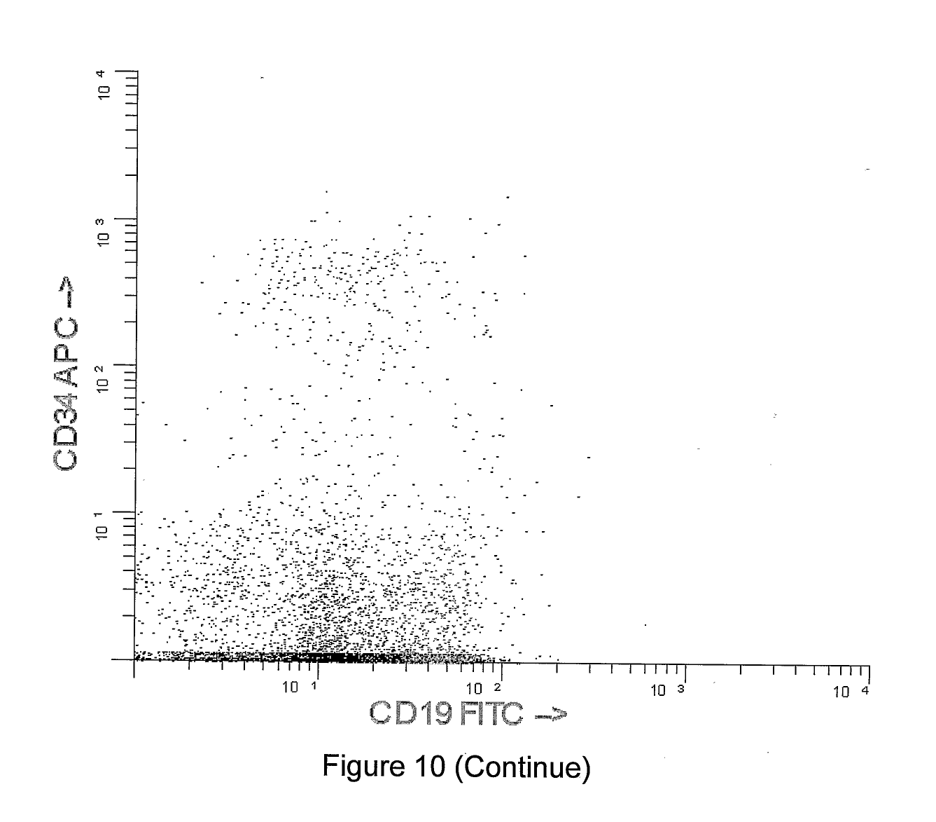

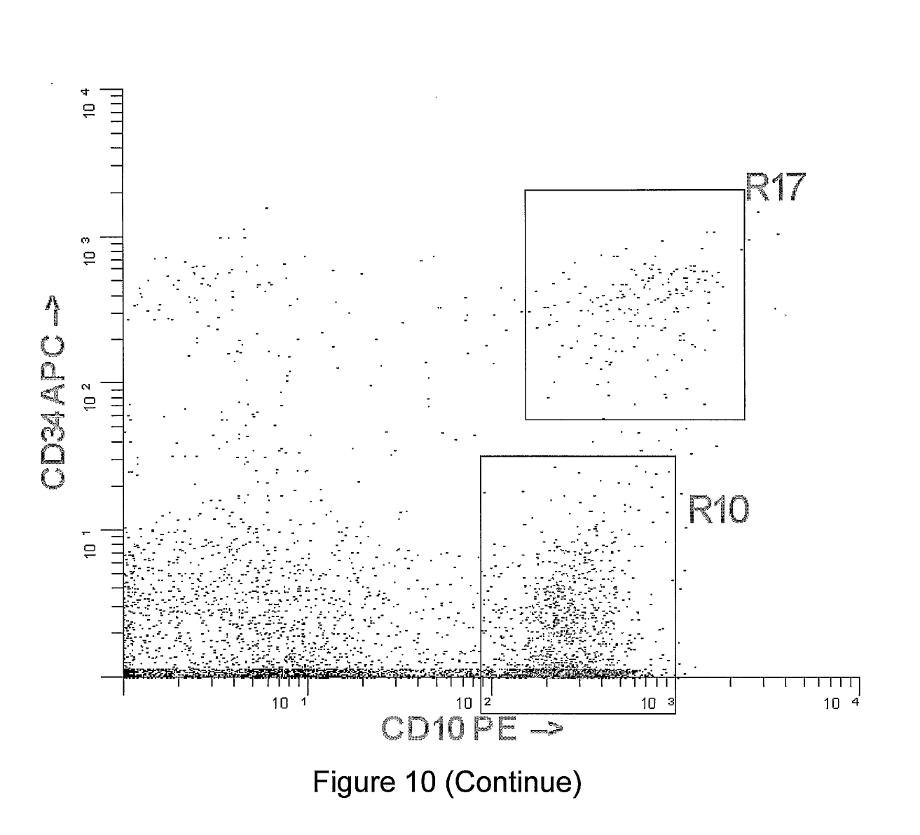

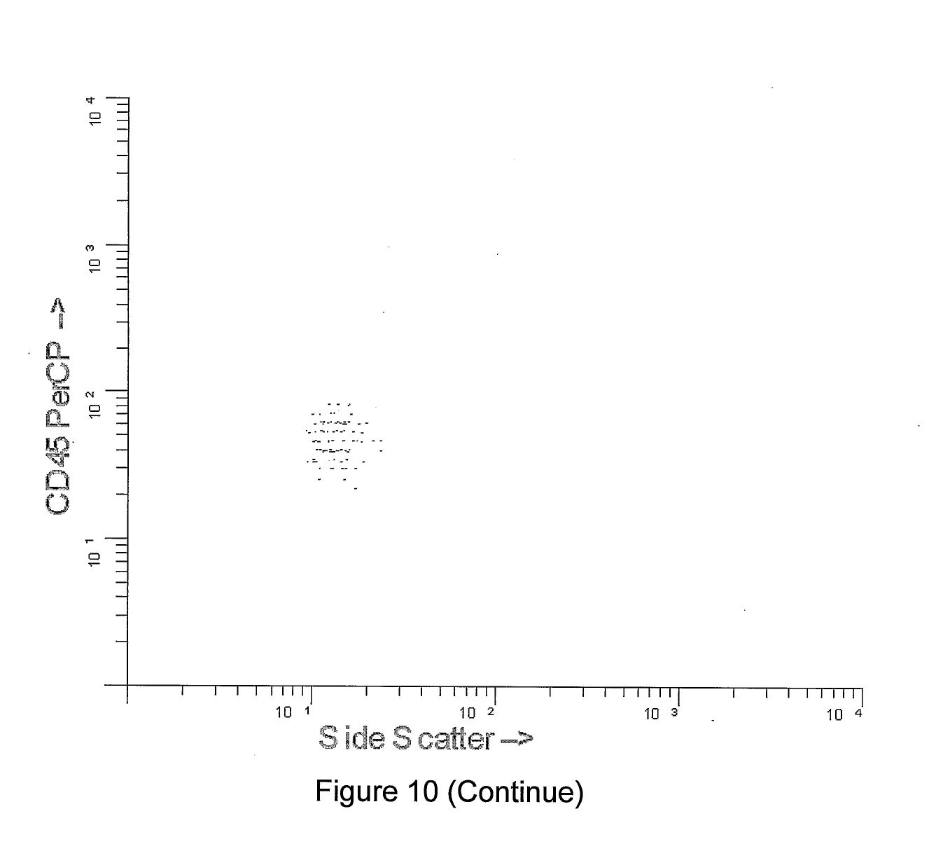

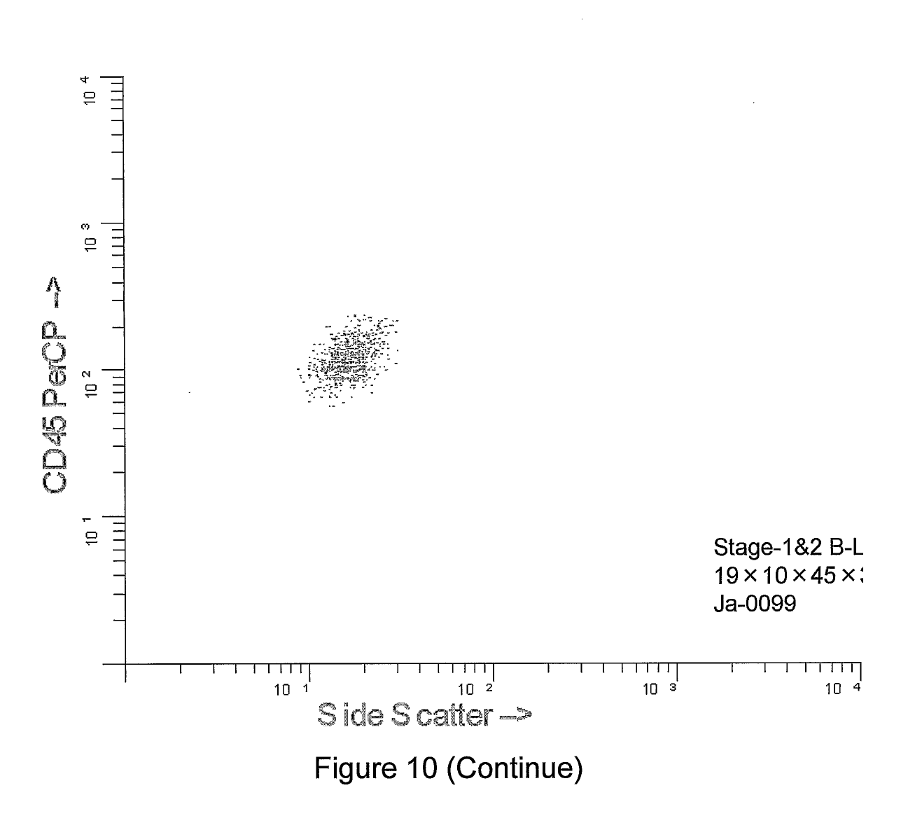

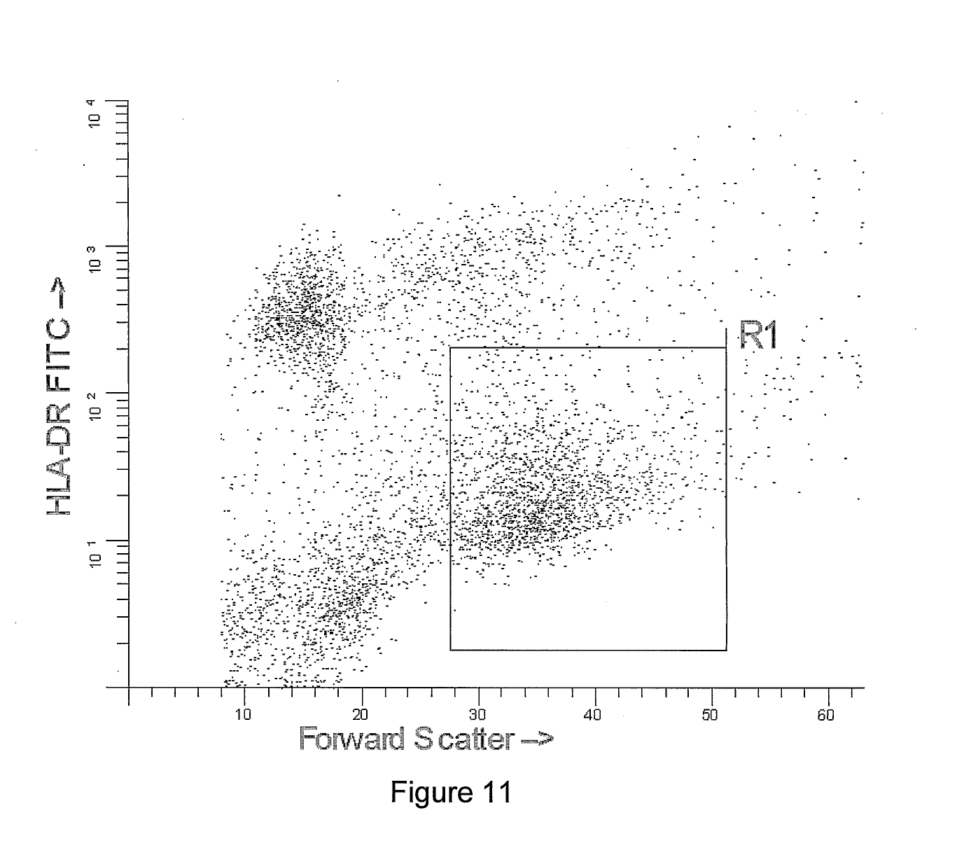

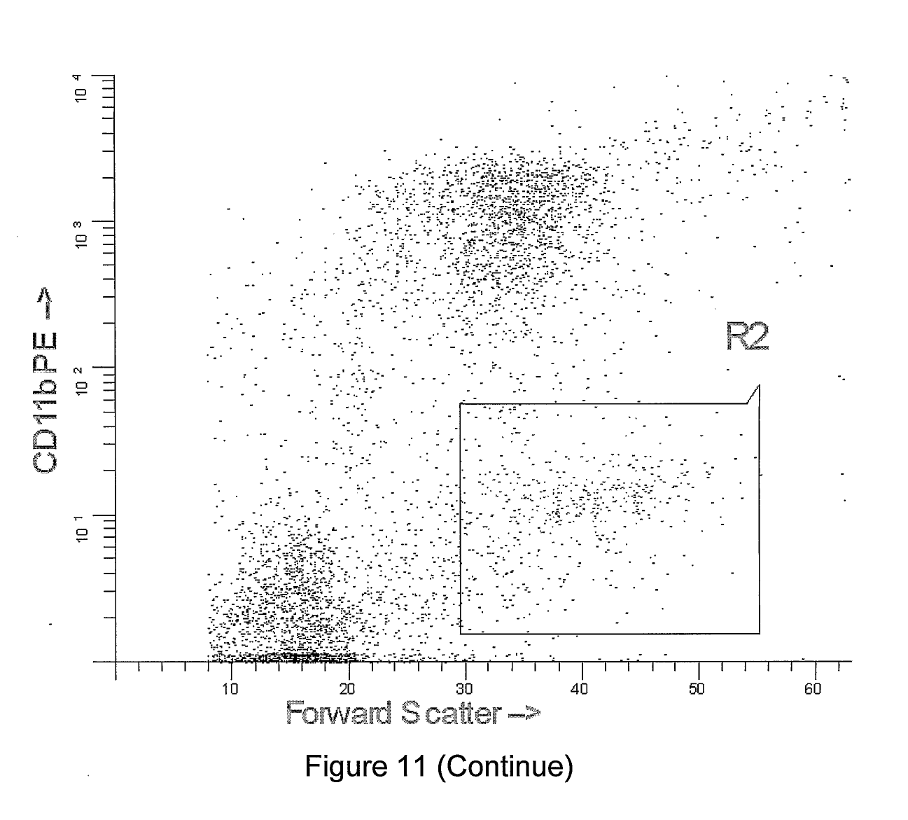

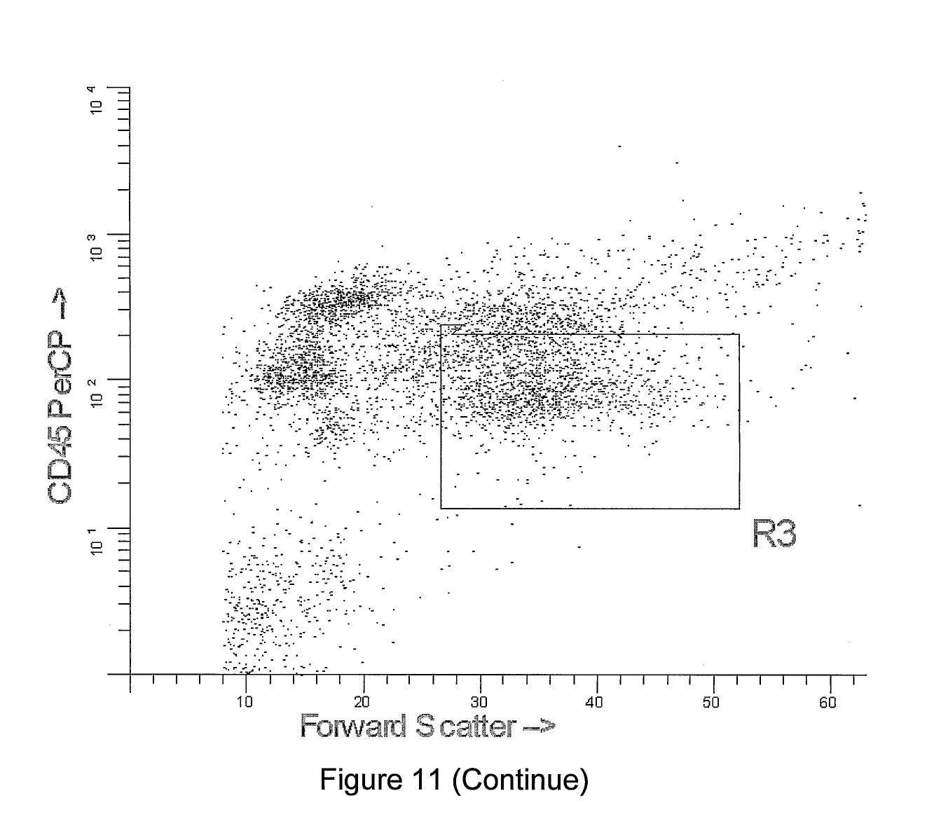

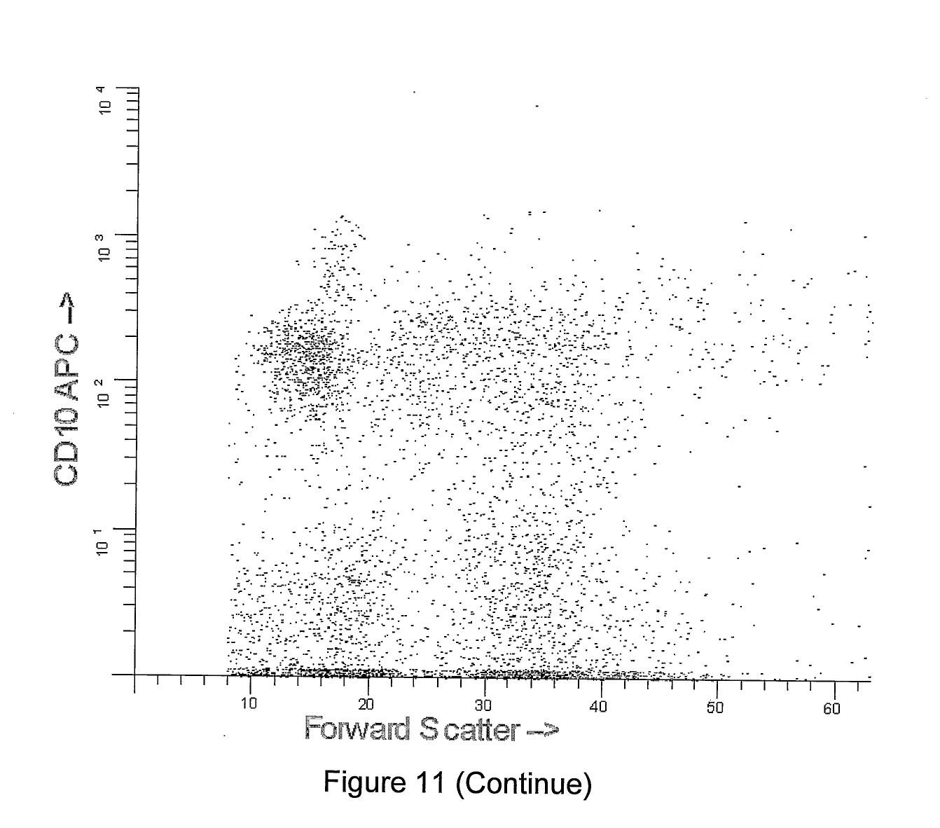

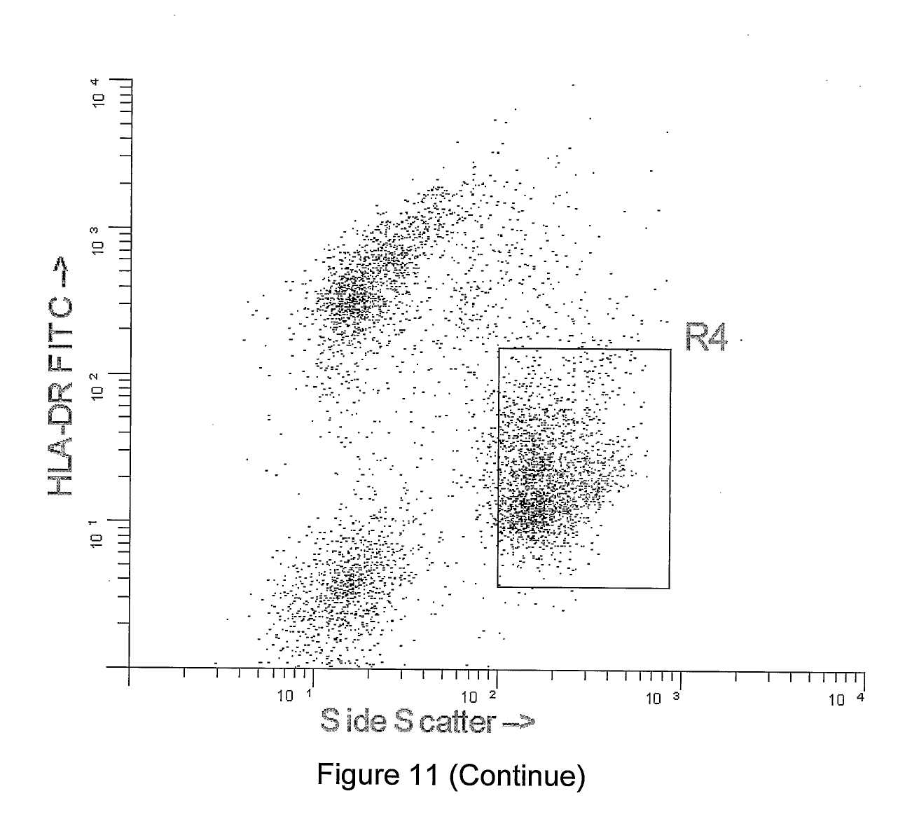

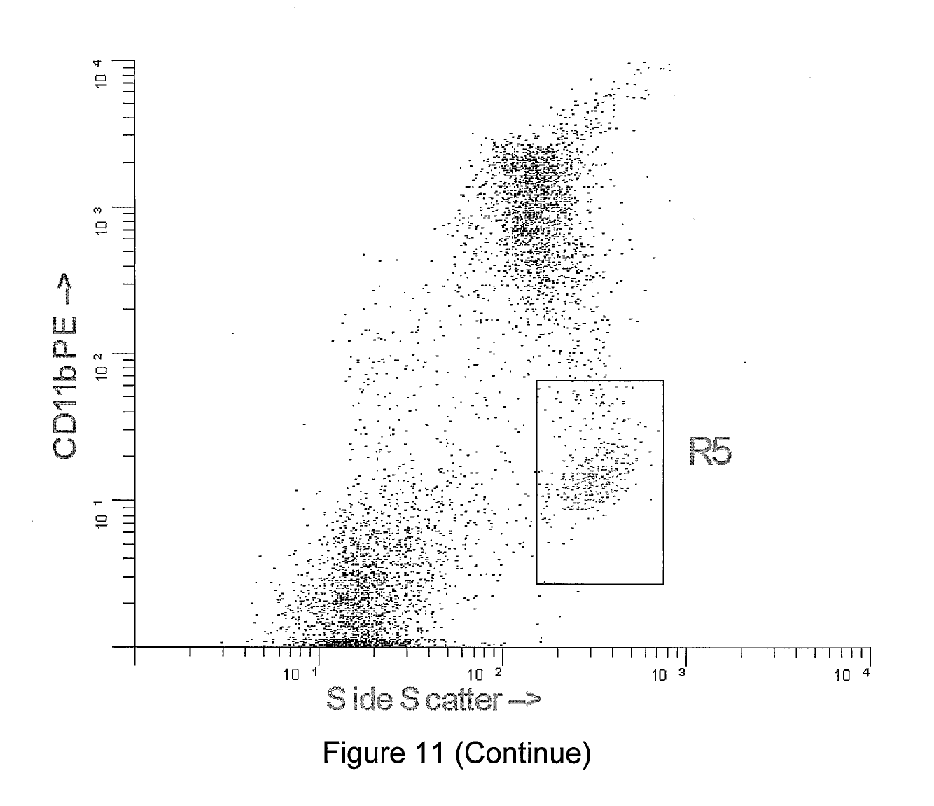

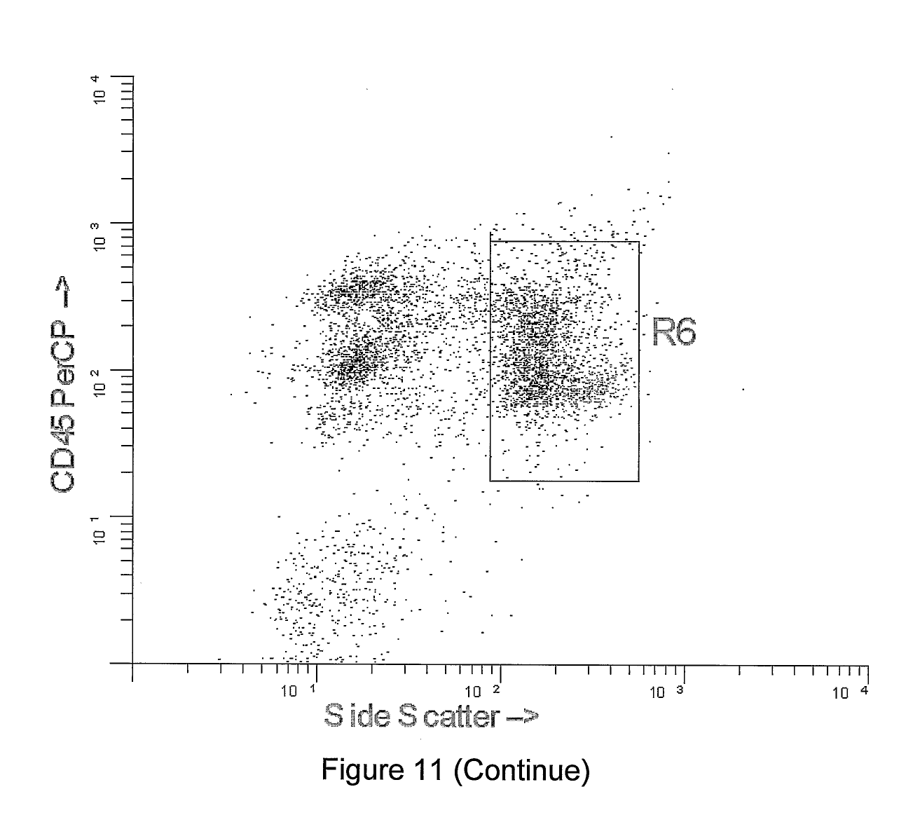

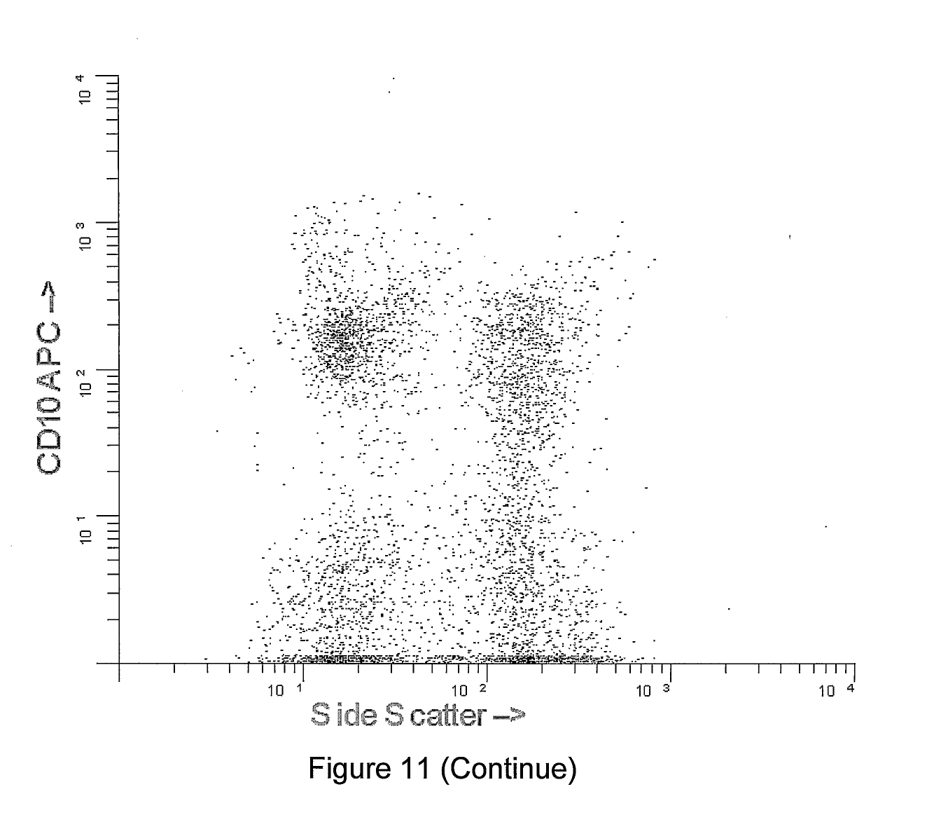

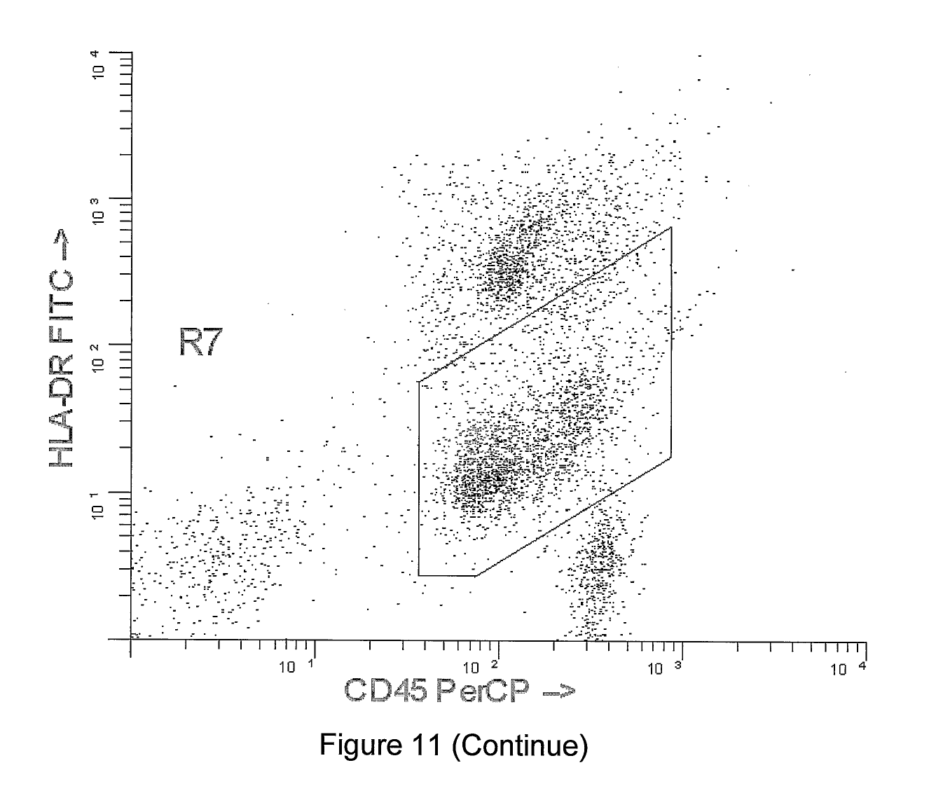

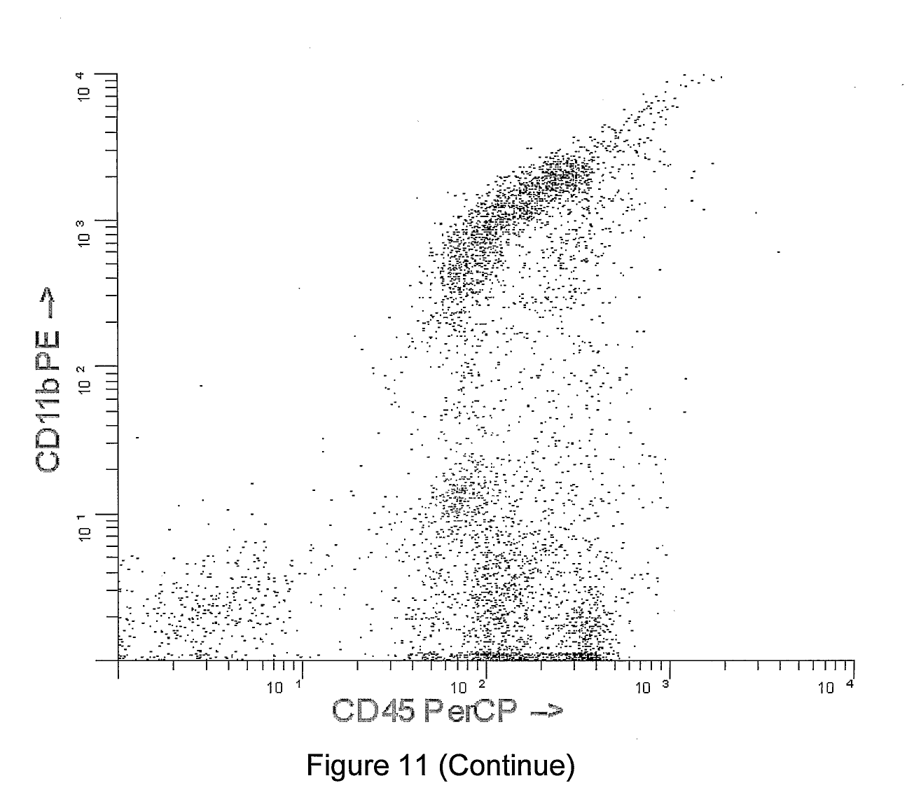

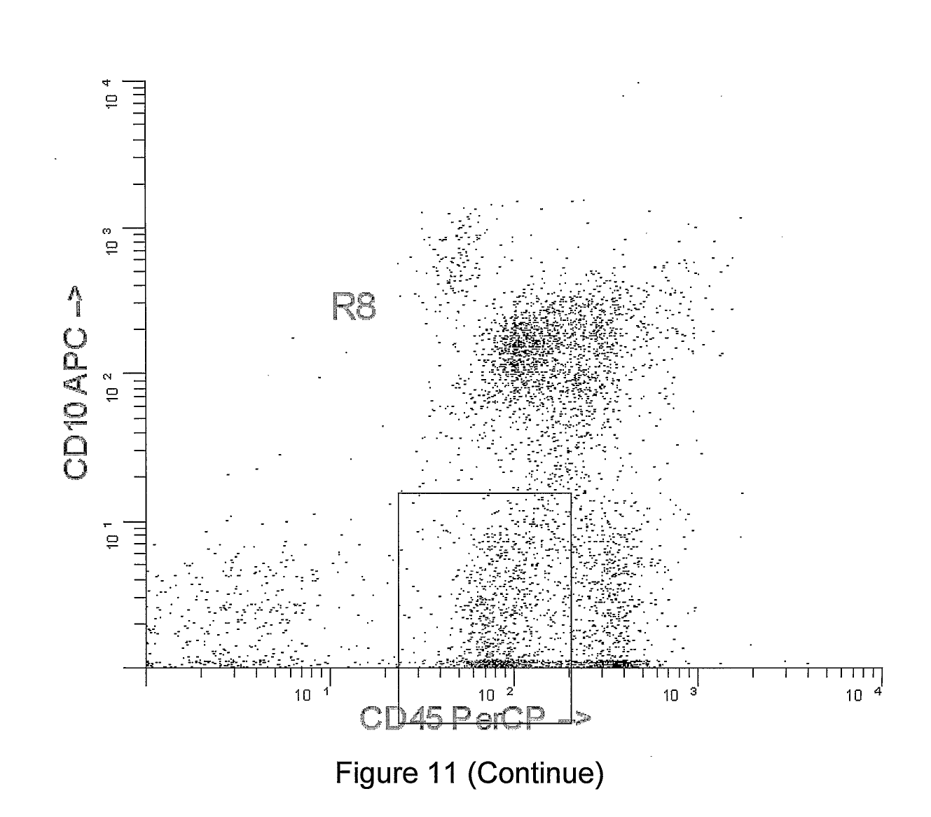

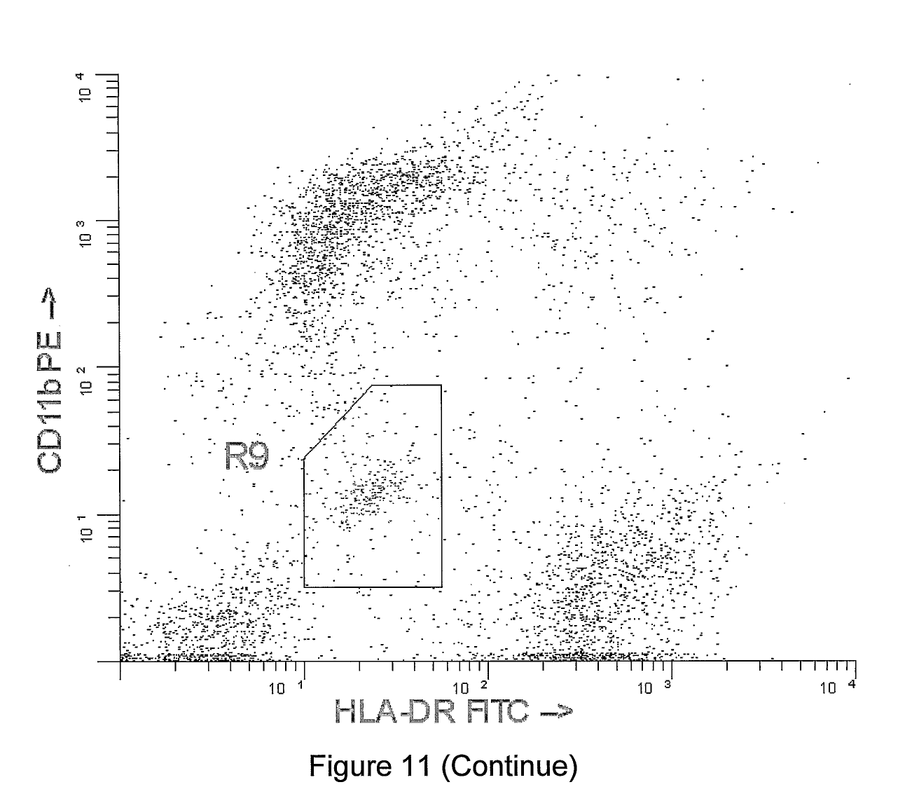

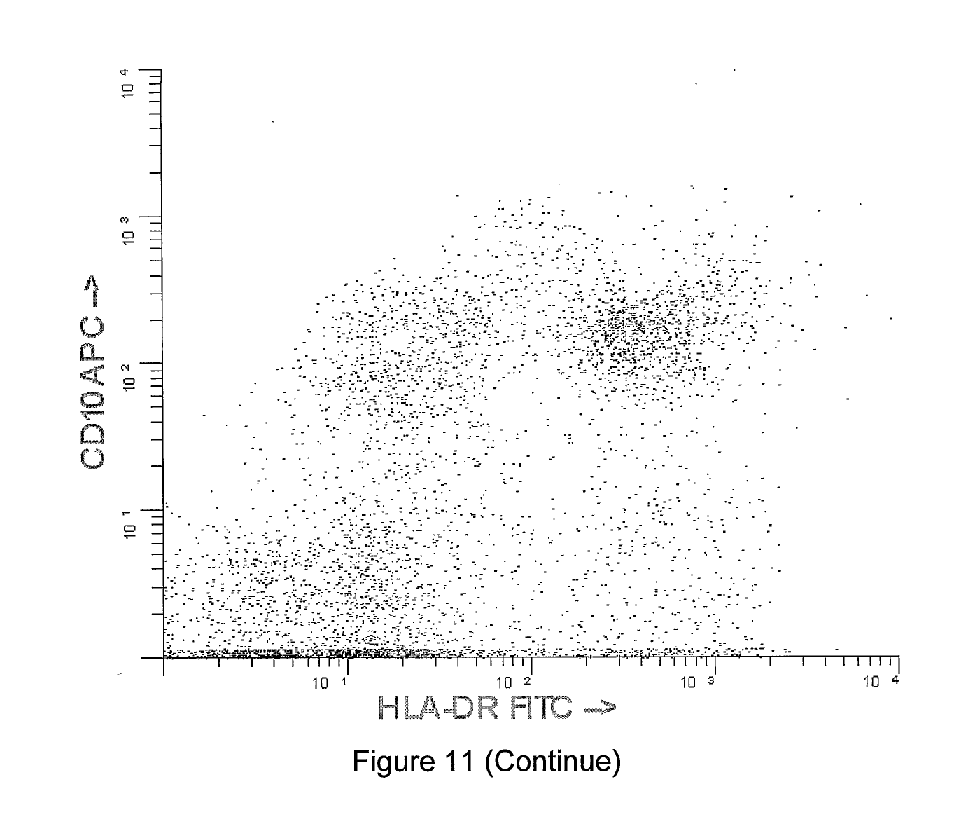

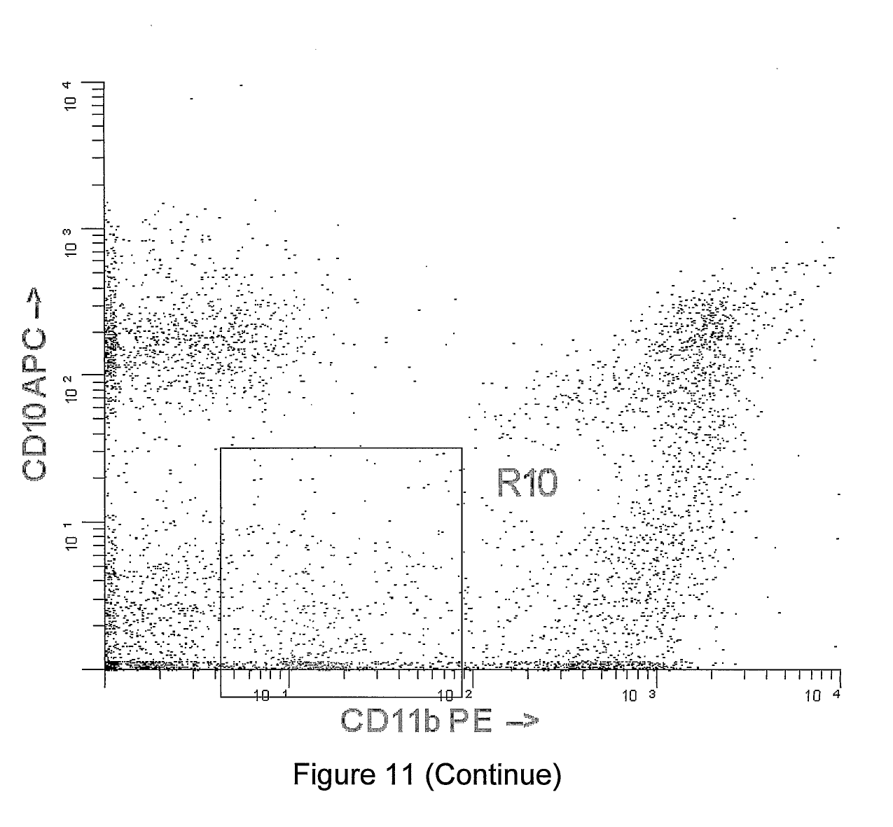

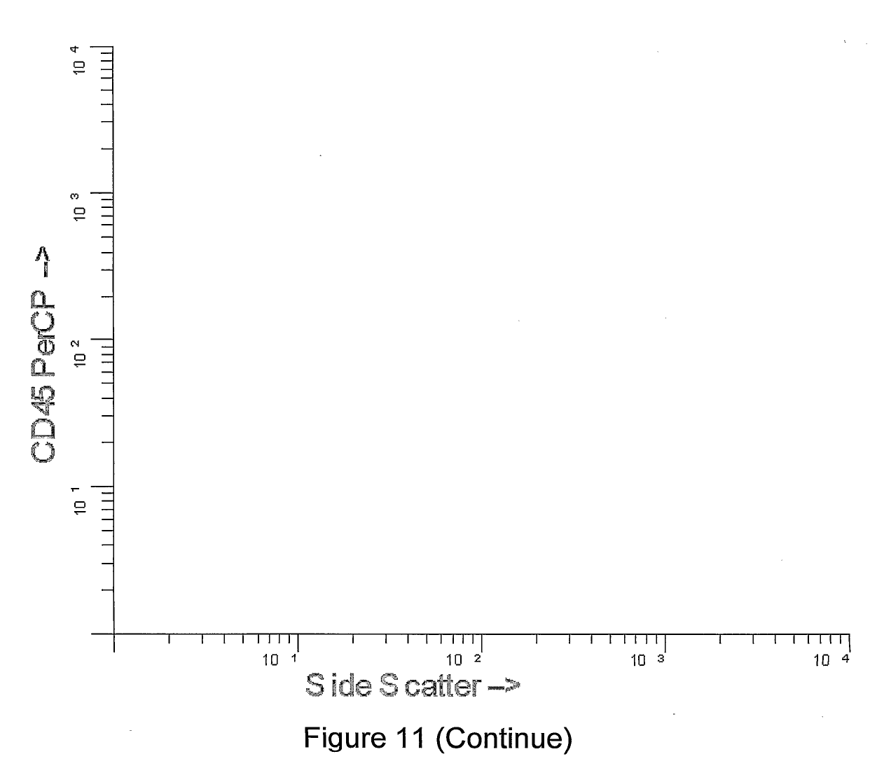

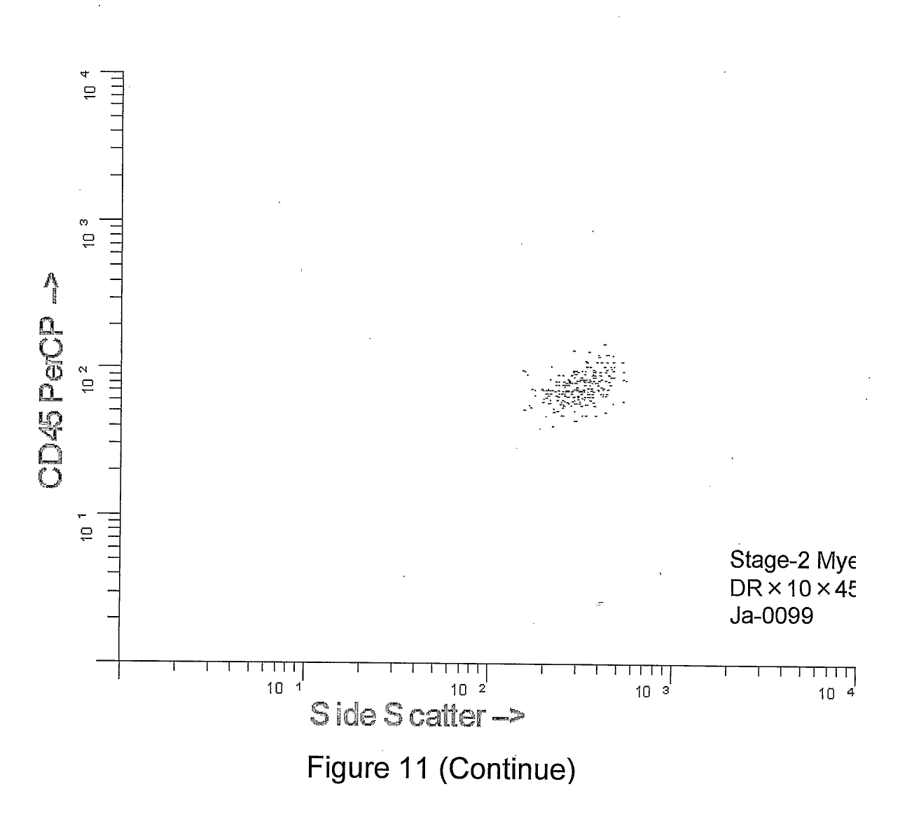

As typical examples, Stage-2 Mono (CD34×CD4×CD45×CD10) (FIG. 5) at Ja-0095 stage, Stage-2 Mono (CD14×CD33×CD45×CD11b) (FIG. 6), Stage-1 and Stage-2 B-Lymph (CD19×CD10×CD45×CD34) at Ja-0096 stage (FIG. 7), Stage-2 Myelo (HLA-DR×CD10×CD11b) (FIG. 8) and Stage-4 Myelo (HLA-DR×CD10×CD45×CD11b) (FIG. 9), and Stage-1 and Stage-2 B-Lymph at Ja-0099 stage (CD19×CD10×CD45×CD34) (FIG. 10) and Stage-2 Myelo (HLA-DR×CD10×CD11b) (FIG. 11) are shown.

As shown in figures, it is clear that acute leukemia cells were observed as 0.7% of whole leukocyte. Condition of the patient after Ja-0104 is shown.

With respect to the condition of the patient after Ja-0095, May 24, 2004 (Ja-0111), acute leukemia cells remained as 0.06% of whole leukocyte, however, other bone marrow blood cells started normal remodeling. At Jul. 1, 2004 (Ja-0131), acute leukemia cells were not observed and immature bone marrow blood cells were observed. All those cells were present at Normal Pathway. Thus, acute bone marrow remodeling appeared to start. At Jul. 28, 2004, the patient is still alive without symptom.

Accordingly, diagnosis in detail allowed appropriate treatment.

### Example 6

In another patient, similar diagnosis and treatment were carried out. The patient is afflicted by an acute leukemia.

Jan. 20, 2004 Ja-0048 (during remission treatment: 2.1% lymphocyte, 2.3% monocyte, 51.9% bone marrow form, 31.6% lymphoblast, and 4.2% myeloblast. Acute leukemia cells were not observed. Many lymphoblasts were observed. In a morphology test, remission is defined as a condition where blast cells in bone marrow are 5% or less. The above data (blast cells 31.6%) cause suspicion of recurrence, thus, there may be a need for additional treatment. Analysis of antigen expression pattern using MDF demonstrated that the blast cells are normal immature cells, Stage-1 B lymphoblast and Stage-2 B lymphoblast. Acure remodeling of bone marrow is recognized. Potent additional treatment is necessary. Such treatment may threaten the life of the patient. Current treatment should be continued.

Mar. 16, 2004 Ja-0073 (observing progress): 34.8% lymphocyte, 8.9% monocyte, 22.1% bone marrow form, 10.8% lymphoblast, and 12.1% myeloblast. Acute leukemia cells were not observed. As the previous diagnosis, many lymphoblasts were observed. According to the antigen expression pattern, the lymphoblasts were identified as normal immature cells, Stage-1 B lymphoblast. Similarly, many myeloblasts were observed. Small numbers of mature bone marrow cells were observed. Cells are in the differentiation maturation process of promyelocytes. Acute remodeling of bone marrow has just started. It is necessary to confirm that the Stage-1 B lymphoblasts develop into Stage-2 B lymphoblasts, and that myeloblasts and promyelocyts develop into mature myelocytes, in the future.

May 12, 2004 Ja-0104 (before starting treatment to maintain remission): 5.4% lymphocyte, 5.2% monocyte, 48.4% bone marrow form, 23.1% lymphoblast, and 5.5% myeloblast. Most blast cells showed normal pattern. However, 0.3% of cells, which may be acute leukemia cells, were observed. Immediate onset of treatment to maintain remission is required. Although tests have not been carried out, on Jul. 28, 2004, the patient is still alive without symptoms.

Accordingly, diagnosis in detail allowed appropriate treatment.

Although certain preferred embodiments have been described herein, it is not intended that such embodiments be construed as limitations on the scope of the invention except as set forth in the appended claims. Various other modifications and equivalents will be apparent to and can be readily made by those skilled in the art, after reading the description herein, without departing from the scope and spirit of this invention. All patents, published patent applications and publications cited herein are incorporated by reference as if set forth fully herein.

### INDUSTRIAL APPLICABILITY

The present invention provides information for more appropriately diagnosing diseases such as leukemia, and thus utility should be recognized in a variety of industries relating to pharmaceuticals and medicine.

What is claimed is:

1. A method for identifying a stage of differentiation-maturation of a cell comprising the steps of:
A) measuring an expression level of at least one cellular marker; and
B) determining the stage of differentiation-maturation of the cell based on the expression level.

2. The method according to claim 1, wherein the expression level of the cellular marker is relative.

3. The method according to claim 1, wherein the expression level of the cellular marker is absolute.

4. The method according to claim 1, wherein at least two cellular markers are used as the cellular marker.

5. The method according to claim 1, wherein at least three cellular markers are used as the cellular marker.

6. The method according to claim 1, wherein at least four cellular markers are used as the cellular marker.

7. The method according to claim 1, wherein the cellular markers are selected from the group consisting of: CD1a, CD2, CD3, CD4, CD5, CD7, CD8, CD10, CD11b, CD13, CD14, CD15, CD16, CD19, CD20, CD22, CD23, CD33, CD34, CD45, TdT, FMC7 and HLA-DR.

8. The method according to claim 1, wherein the cell comprises a hematopoietic cell.

9. The method according to claim 1, wherein the cell comprises a cell selected from the group consisting of monocytic lineage cells, myeloid lineage cells, B lymphocyte like-cells, and T lymphocytic lineage cells.

10. The method according to claim 1, wherein the differentiation-maturation stage comprises Stage 1, Stage 2 and Stage 3 for monocytic lineage cells.

11. The method according to claim 1, wherein the differentiation-maturation stage comprises Stage 11 Stage 2, Stage 3, Stage 4 and Stage 5 for myeloid lineage cells.

12. The method according to claim 1, wherein the differentiation-maturation stage comprises Stage 1, Stage 2, Stage 3 and Stage 4 for B lymphocytic lineage cells.

13. The method according to claim 1, wherein the differentiation-maturation stage comprises Stage 1, Stage 2, Stage 3 and Stage 4 for T lymphocytic lineage cells.

14. The method according to claim 1, wherein the cellular marker comprises a combination of lineage specific CD antigens.

15. The method according to claim 1, wherein the cellular marker comprises a combination of antigens which are not lineage specific CD antigens.

16. The method according to claim 1, wherein the cellular marker is, with respect to B lymphocytic lineage, selected from the group consisting of the combination of CD10, CD19, CD34 and CD45; the combination of CD10, CD20, CD5 and CD45; the combination of CD19, CD23, CD5 and CD45; the combination of CD10, CD20, CD34 and CD45; and the combination of CD5, CD19, CD34 and CD45.

17. The method according to claim 1 wherein the cellular marker is, with respect to T lymphocytic lineage, selected from the group consisting of the combination of CD1a, CD3, CD10 and CD45; the combination of CD4, CD8, CD7 and CD45; the combination of CD1a, CD3, CD34 and CD45; and the combination of CD4, CD8, CD10 and CD45.

18. The method according to claim 1, wherein the cellular marker is, with respect to myeloid lineage or monocytic lineage, selected from the group consisting of the combination of HLA-DR, CD11b, CD10 and CD45; the combination of CD16, CD13, CD33 and CD45; the combination of CD4, CD10, CD34 and CD45; the combination of CD11b, CD13, CD16 and CD45; and the combination of CD10, CD4, CD14 and CD45.

19. A system for identifying a differentiation/maturation stage of a cell, comprising:
A) means for measuring an expression level of at least one cellular marker; and
B) means for determining the differentiation/maturation stage of the cell based on the expression level.

20. The system according to claim 19, wherein the means for measuring measures a relative level of an expression of a cellular marker.

21. The system according to claim 19, wherein the means for measuring measures an absolute level of an expression of a cellular marker.

22. The system according to claim 19, wherein the means for measuring comprises an agent specific to a cellular marker.

23. The system according to claim 22, wherein the cellular marker comprises at least two cellular markers.

24. The system according to claim 22, wherein the cellular marker comprises at least three cellular markers.

25. The system according to claim 22, wherein the cellular marker comprises at least four cellular markers.

26. The system according to claim 22, wherein the cellular marker is selected from the group consisting of CD1a, CD2, CD3, CD4, CD5, CD7, CD8, CD10, CD11b, CD13, CD14, CD15, CD16, CD19, CD20, CD22, CD23, CD33, CD34, CD45, TdT, FMC7 and HLA-DR.

27. The system according to claim 19, wherein the cell comprises a hematopoietic stem cell.

28. The system according to claim 19, wherein the cell comprises a cell selected from the group consisting of a monocytic lineage cell, a B lymphocytic lineage cell, a T lymphocytic lineage cell lineage and a myeloid lineage cell.

29. The system according to claim 19, wherein the differentiation/maturation stage comprises Stage 1, Stage 2 and Stage 3 for monocytic lineage.

30. The system according to claim 19, wherein the differentiation/maturation stage comprises Stage 1, Stage 2, Stage 3, Stage 4 and Stage 5 for myeloid lineage.

31. The system according to claim 19, wherein the differentiation/maturation stage comprises Stage 1, Stage 2, Stage 3 and Stage 4 for B lymphocytic lineage lineage.

32. The system according to claim 19, wherein the differentiation/maturation stage comprises Stage 1, Stage 2, Stage 3 and Stage 4 for T lymphocytic lineage lineage.

33. The system according to claim 22, wherein the cellular marker comprises a combination of antigens which are lineage specific CD antigens.

34. The system according to claim 22, wherein the cellular marker comprises a combination of antigens which are not lineage specific CD antigens.

35. The system according to claim 22, wherein the cellular marker is, with respect to B lymphocytic lineage, selected from the group consisting of the combination of CD10, CD19, CD34 and CD45; the combination of CD10, CD20, CD5 and CD45; the combination of CD19, CD23, CD5 and CD45; the combination of CD10, CD20, CD34 and CD45; and the combination of CD5, CD19, CD34 and CD45.

36. The system according to claim 22, wherein the cellular marker is, with respect to T lymphocytic lineage, selected from the group consisting of the combination of CD1a, CD3, CD10 and CD45; the combination of CD4, CD8, CD7 and CD45; the combination of CD1a, CD3, CD34 and CD45; and the combination of CD4, CD8, CD10 and CD45.

37. The system according to claim 22, wherein the cellular marker is, with respect to bone-marrow lineage or monocytic lineage, selected from the group consisting of the combination of HLA-DR, CD11b, CD10 and CD45; the combination of CD16, CD13, CD33 and CD45; the combination of CD4, CD10, CD34 and CD45; the combination of CD11b, CD13, CD16 and CD45; and the combination of CD10, CD4, CD14 and CD45.

38. The system according to claim 19, wherein the means for measuring comprises a flow cytometer.

39. A method for determining a differentiation/maturation stage of a cell, comprising the steps of:
A) providing a measurement pattern (“normal pattern”) with respect to a normal cell having a normal differentiation/measurement stage of the cell, in a cytometry of an expression level of at least one cellular marker; and
B) determining the differentiation/maturation stage of the cell by comparing the pattern in a flow cytometry of an expression level of the cellular marker compared with the normal pattern.

40. The system for determining a differentiation/maturation stage of a cell comprising:
A) means for providing measurement pattern (“normal pattern”) with respect to a normal cell having a normal differentiation/measurement stage of the cell, in a cytometry of an expression level of at least one cellular marker; and
B) means for determining the differentiation/maturation stage of the cell by comparing the pattern in a flow cytometry of an expression level of the cellular marker compared with the normal pattern.

41. A method for determining whether a differentiation/maturation stage of a cell is normal or not, comprising the steps of:
A) measuring an expression level of at least one cellular marker in a flow cytometry; and
B) comparing a pattern of the expression level in a flow cytometry, with an expression level of the cellular marker presented by a cell in a normal differentiation/maturation stage in a flow cytometry, wherein the fact that there is difference therebetween is indicative of an abnormal cell.

42. A system for determining whether a differentiation/maturation stage of a cell is normal or not, comprising:
A) means for measuring an expression level of at least one cellular marker in a flow cytometry; and
B) means for comparing a pattern of the expression level in a flow cytometry, with an expression level of the cellular marker presented by a cell in a normal differentiation/maturation stage in a flow cytometry, wherein the fact that there is difference therebetween is an indicative of an abnormal cell.

43. A method for treating a subject based on a differentiation/maturation stage of a cell, comprising the steps of:
A) measuring an expression level of at least one cellular marker;
B) determining the differentiation/maturation stage of the cell based on the expression level; and
C) providing to the subject an appropriate treatment for the determined differentiation/maturation stage.

44. A method for treating a subject based on a differentiation/maturation stage of a cell, comprising the steps of:
A) measuring an expression level of at least one cellular marker;
B) determining the differentiation/maturation stage of the cell based on the expression level; and
C) providing to the subject an appropriate treatment based on the result of comparing the determined differentiation/maturation stage, and a stage which the cell takes in its normal stage.

45. The method according to claim 44, wherein said appropriate treatment comprises at least one selected from the group consisting of:
i) a treatment of continuing the present therapy or termination of the present therapy and to conduct follow-up only, when a normal cell exists or a cell present is determined to be a normal immature cell with respect to the determined differentiation/maturation stage of the cell; and
ii) a treatment in which the enhancement or alteration of the present therapy when the present cell is an abnormal cell with respect to the determined differentiation/maturation stage of the cell.

46. The method according to claim 44, wherein the treatment comprises a treatment in which an additional stem cell transplantation is conducted when depletion of the original stem cell or an abnormality of differentiation/maturation progress is recognized, even if the existing cell is a normal immature cell with respect to the determined differentiation/maturation stage of the cell.

47. A system for treating a subject based on a differentiation/maturation stage of a cell, comprising the steps of:
A) means for measuring an expression level of at least one cellular marker;
B) means for determining the differentiation/maturation stage of the cell based on the expression level; and
C) means for providing to the subject an appropriate treatment for the determined differentiation/maturation stage.

48. A system for providing a subject with an appropriate treatment based on a differentiation/maturation stage of a cell, comprising:
A) means for measuring an expression level of at least one cellular marker;
B) means for determining the differentiation/maturation stage of the cell based on the expression level; and
C) means for providing the subject with an appropriate treatment based on the determined differentiation/maturation stage.

49. A system for treating a subject based on a differentiation/maturation stage of a cell, comprising the steps of:
A) means for measuring an expression level of at least one cellular marker;
B) means for determining the differentiation/maturation stage of the cell based on the expression level; and
C) means for providing to the subject an appropriate treatment based on the result of comparing the determined differentiation/maturation stage, and a stage which the cell takes in its normal stage.

50. A system for providing a subject with an appropriate treatment based on a differentiation/maturation stage of a cell, comprising:
A) means for measuring an expression level of at least one cellular marker;
B) means for determining the differentiation/maturation stage of the cell based on the expression level; and
C) means for providing the subject with an appropriate treatment based on the result of comparing the determined differentiation/maturation stage, and a stage which the cell takes in its normal stage.

51. A pattern of differentiation/maturation of a cell produced by a method according to claim 1.
